(19) United States
(12) Patent Application Publication
(10) Pub. No.: US 2016/0287589 A1
(43) Pub. Date: Oct. 6, 2016

(54) SUBSTITUTED-IMIDAZOPYRIDAZINES

(71) Applicant: BAYER PHARMA AKTIENGESELLSCHAFT, Berlin (DE)

(72) Inventors: Knut FIS, Berlin (DE); Florian PUEHLER, Massachusetts (DE); Ludwig ZORN, Berlin (DE); Volker SCHULZE, OT Bergf (DE); Detlev SÜLZLE, Berlin (DE); Philip LIENAU, Berlin (DE); Antje Margret WENGNER, Berlin (DE); Kirstin PETERSEN, Berlin (DE); Ulf BÖMER, Glienicke (DE)

(21) Appl. No.: 14/769,091

(22) PCT Filed: Feb. 18, 2014
(86) PCT No.: PCT/EP2014/053056

§ 371 Date: Aug. 19, 2015

(30) Foreign Application Priority Data
Feb. 20, 2013 (EP) 13155974.2
Feb. 20, 2013 (EP) 13155978.3
Mar. 6, 2013 (EP) 13157920.3
Mar. 6, 2013 (EP) 13157926.0
Mar. 6, 2013 (EP) 13157932.8
Mar. 6, 2013 (EP) 13157935.1

(51) Int. Cl. A61K 31/5025; C07D 487/04; A61K 31/5377; C07D 519/00; A61K 9/20; A61K 31/695; A61K 9/19; A61K 9/00; A61K 9/48; A61K 45/06; C07F 7/18

(52) U.S. Cl. CPC A61K 31/5025; A61K 45/06; C07D 487/04; A61K 31/5377; C07D 519/00; C07F 7/1856; A61K 31/695; A61K 9/19; A61K 9/0019; A61K 9/48; A61K 9/20

(57) ABSTRACT

The present invention relates to amido-substituted imidazopyridazine compounds of general formula (I): (Ia) (Ib) (Ic) (Id) in which A, Y, R1, R2, R3, R4 and n are as defined in the claims, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative and/or angiogenesis disorder, as a sole agent or in combination with other active ingredients.

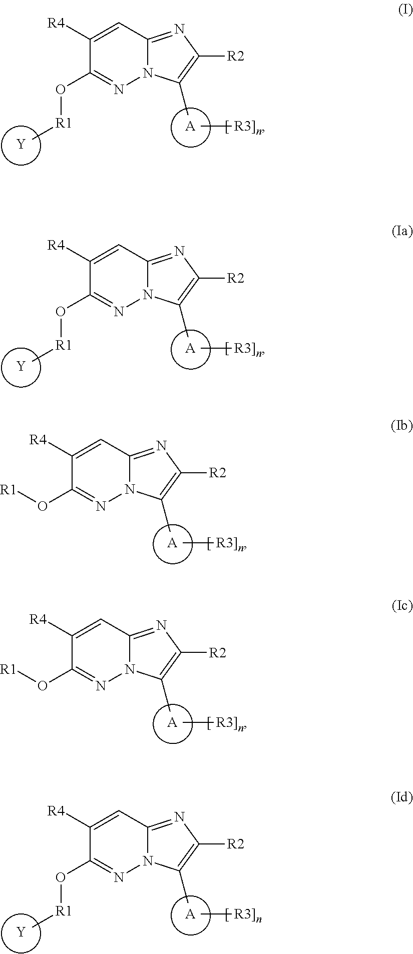

The present invention relates to substituted imidazopyridazine compounds of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative and/or angiogenesis disorder, as a sole agent or in combination with other active ingredients.

### BACKGROUND OF THE INVENTION

The present invention relates to chemical compounds that inhibit MKNK1 kinase (also known as MAP Kinase interacting Kinase, Mnk1) and MKNK2 kinase (also known as MAP Kinase interacting Kinase, Mnk2). Human MKNKs comprise a group of four proteins encoded by two genes (Gene symbols: MKNK1 and MKNK2) by alternative splicing. The b-forms lack a MAP kinase-binding domain situated at the C-terminus. The catalytic domains of the MKNK1 and MKNK2 are very similar and contain a unique DFD (Asp-Phe-Asp) motif in subdomain VII, which usually is DFG (Asp-Phe-Gly) in other protein kinases and suggested to alter ATP binding [Jauch et al., Structure 13, 1559-1568, 2005 and Jauch et al., EMBO J25, 4020-4032, 2006]. MKNK1a binds to and is activated by ERK and p38 MAP Kinases, but not by JNK1. MKNK2a binds to and is activated only by ERK. MKNK1b has low activity under all conditions and MKNK2b has a basal activity independent of ERK or p38 MAP Kinase. [Buxade M et al., Frontiers in Bioscience 5359-5374, May 1, 2008]

MKNKs have been shown to phosphorylate eukaryotic initiation factor 4E (eIF4E), heterogeneous nuclear RNA-binding protein A1 (hnRNP A1), polypyrimidine-tract binding protein-associated splicing factor (PSF), cytoplasmic phospholipase A2 (cPLA2) and Sprouty 2 (hSPRY2) [Buxade M et al., Frontiers in Bioscience 5359-5374, May 1, 2008].

eIF4E is an oncogene that is amplified in many cancers and is phosphorylated exclusively by MKNKs proteins as shown by KO-mouse studies [Konicek et al., Cell Cycle 7:16, 2466-2471, 2008; Ueda et al., Mol Cell Biol 24, 6539-6549, 2004]. eIF4E has a pivotal role in enabling the translation of cellular mRNAs. eIF4E binds the 7-methylguanosine cap at the 5′ end of cellular mRNAs and delivers them to the ribosome as part of the eIF4F complex, also containing eIF4G and eIF4A. Though all capped mRNAs require eIF4E for translation, a pool of mRNAs is exceptionally dependent on elevated eIF4E activity for translation. These so-called “weak mRNAs” are usually less efficiently translated due to their long and complex 5′UTR region and they encode proteins that play significant roles in all aspects of malignancy including VEGF, FGF-2, c-Myc, cyclin D1, survivin, BCL-2, MCL-1, MMP-9, heparanase, etc. Expression and function of eIF4E is elevated in multiple human cancers and directly related to disease progression [Konicek et al., Cell Cycle 7:16, 2466-2471, 2008].

MKNK1 and MKNK2 are the only kinases known to phosphorylate eIF4E at Ser209. Overall translation rates are not affected by eIF4E phosphorylation, but it has been suggested that eIF4E phosphorylation contributes to polysome formation (i.e. multiple ribosome on a single mRNA) that ultimately enables more efficient translation of “weak mRNAs” [Buxade M et al., Frontiers in Bioscience 5359-5374, May 1, 2008]. Alternatively, phosphorylation of eIF4E by MKNK proteins might facilitate eIF4E release from the 5′ cap so that the 48S complex can move along the “weak mRNA” in order to locate the start codon [Blagden S P and Willis A E, Nat Rev Clin Oncol. 8(5):280-91, 2011]. Accordingly, increased eIF4E phosphorylation predicts poor prognosis in non-small cell lung cancer patients [Yoshizawa et al., Clin Cancer Res. 16(1):240-8, 2010]. Further data point to a functional role of MKNK1 in carcinogenesis, as overexpression of constitutively active MKNK1, but not of kinase-dead MKNK1, in mouse embryo fibroblasts accelerates tumor formation [Chrestensen C. A. et al., Genes Cells 12, 1133-1140, 2007]. Moreover, increased phosphorylation and activity of MKNK proteins correlate with overexpression of HER2 in breast cancer [Chrestensen, C. A. et al., J. Biol. Chem. 282, 4243-4252, 2007]. Constitutively active, but not kinase-dead, MKNK1 also accelerated tumor growth in a model using Eμ-Myc transgenic hematopoietic stem cells to produce tumors in mice. Comparable results were achieved, when an eIF4E carrying a S209D mutation was analyzed. The S209D mutation mimicks a phosphorylation at the MKNK1 phosphorylation site. In contrast a non-phosphorylatable form of eIF4E attenuated tumor growth [Wendel H G, et al., Genes Dev. 21(24):3232-7, 2007]. A selective MKNK inhibitor that blocks eIF4E phosphorylation induces apoptosis and suppresses proliferation and soft agar growth of cancer cells in vitro. This inhibitor also suppresses outgrowth of experimental B16 melanoma pulmonary metastases and growth of subcutaneous HCT116 colon carcinoma xenograft tumors without affecting body weight [Konicek et al., Cancer Res. 71(5):1849-57, 2011]. In summary, eIF4E phosphorylation through MKNK protein activity can promote cellular proliferation and survival and is critical for malignant transformation. Inhibition of MKNK activity may provide a tractable cancer therapeutic approach.

WO 2007/025540 A2 (Bayer Schering Pharma AG) relates to substituted imidazo[1,2-b]pyridazines as kinase inhibitors, particularly PKC (protein kinase C) inhibitors, in particular PKC theta inhibitors.

WO 2007/025090 A2 (Kalypsis, Inc.) relates to heterocyclic compounds useful as inhibitors of Mitogen-activated protein kinase (MAPK)/Extracellular signal-regulated protein kinase (Erk) Kinase (abbreviated to “MEK”). In particular, WO 2007/025090 A2 relates inter alia to imidazo[1,2-b]pyridazines.

WO 2007/013673 A1 (Astellas Pharma Inc.) relates to fused heterocycles as inhibitors of Lymphocyte protein tyrosine kinase (abbreviated to “LCK”). In particular, WO 2007/013673 A1 relates inter alia to imidazo[1,2-b]pyridazines.

WO 2007/147646 A1 (Bayer Schering Pharma AG) relates to oxo-substituted imidazo[1,2-b]pyridazines as kinase inhibitors, particularly PKC (protein kinase C) inhibitors, in particular PKC theta inhibitors.

WO 2008/025822 A1 (Cellzome (UK) Ltd.) relates to diazolodiazine derivatives as kinase inhibitors. In particular, WO 2008/025822 A1 relates inter alia to imidazo[1,2-b]pyridazines as kinase inhibitors, particularly inducible T cell kinase (abbreviated to “Itk”) inhibitors.

WO 2008/030579 A2 (Biogen Idec MA Inc.) relates to modulators of interleukin-1 (IL-1) receptor-associated kinase (abbreviated to “IRAK”). In particular, WO 2008/030579 A2 relates inter alia to imidazo[1,2-b]pyridazines.

WO 2008/058126 A2 (Supergen, Inc.) relates inter alia to imidazo[1,2-b]pyridazine derivatives as protein kinase inhibitors, particularly PIM kinase inhibitors.

WO 2009/060197 A1 (Centro Nacional de Investigaciones Oncologicas (CNIO)) relates to imidazopyridazines as protein kinase inhibitors, such as the PIM family kinases.

U.S. Pat. No. 4,408,047 (Merck & Co., Inc.,) relates inter alia to imidazopyridazines having a 3-amino-2-OR-propoxy substituent having beta-adrenergic blocking activity.

WO 03/018020 A1 (Takeda Chemical Industries, Ltd.) relates to inhibitors against c-Jun N-terminal kinase, containing compounds which are, inter alia, imidazo[1,2-b]-pyridazines.

WO 2008/052734 A1 (Novartis AG) relates to heterocyclic compounds as antiinflammatory agents. In particular said compounds are, inter alia, imidazo[1,2-b]pyridazines. The compounds are useful for treating diseases mediated by the ALK-5 and/or ALK-4 receptor, and are also useful for treating diseases mediated by the PI3K receptor, the JAK-2 receptor and the TRK receptor.

WO 2008/072682 A1 (Daiichi Sankyo Company, Limited) relate to imidazo[1,2-b]pyridazine derivative which has an action of inhibiting TNF-alpha production, exerts an effect in a pathological model of inflammatory disease and/or auto-immune disease.

WO 2008/079880 A1 (Alcon Research, Ltd.) relates to 6-aminoimidazo[1,2-b]pyridazine analogues as Rho-kinase inhibitors for the treatment of glaucoma and ocular hypertension.

WO 2009/091374 A2 (Amgen Inc.) relates to fused heterocyclic deriviatives. Selected compounds are effective for prophylaxis and treatment of diseases, such as hepatocyte growth factor (“HGF”) diseases.

WO 2013/013188 A1 (Tolero Pharmaceuticals, Inc.) relates to heterocyclic derivatives for the treatment of cancer, autoimmune, inflammatory and other Pim kinase-associated conditions.

In J. Med. Chem., 2005, 48, 7604-7614, is an article entitled “Structural Basis of Inhibitor Specificity of the Protooncogene Proviral Insertion Site in Moloney Murine Leukemia Virus (PIM-1) Kinase”, and discloses, inter alia, imidazo[1,2-b]pyridazines as inhibitor structures used in the study described therein.

In J. Med. Chem., 2010, 53, 6618-6628, is an article entitled “Discovery of Mitogen-Activated Protein Kinase-Interacting Kinase 1 Inhibitors by a Comprehensive Fragment-Oriented Virtual Screening Approach”, and discloses, inter alia, in Table 1, some specific imidazo[1,2-b]pyridazines as compounds identified as MKNK-1 inhibitors.

In Cancer Res Mar. 1, 2011, 71, 1849-1857 is an article entitled “Therapeutic inhibition of MAP kinase interacting kinase blocks eukaryotic initiation factor 4E phosphorylation and suppresses outgrowth of experimental lung metastases”, and discloses, inter alia, that the known antifungal agent Cercosporamide is an inhibitor of MKNK1.

However, the state of the art described above does not describe the specific substituted imidazopyridazine compounds of general formula (I) of the present invention as defined herein, i.e. an imidazo[1,2-b]pyridazinyl moiety, bearing:
in its 3-position, a:

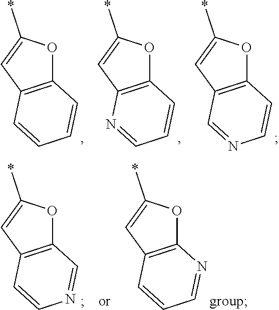

or a group of structure:

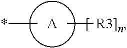

wherein
* indicates the point of attachment of said group with the rest of the molecule, and
A, R3 and n are defined herein;
in its 6-position, a

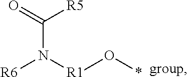

or a

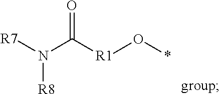

wherein R1, R5, R6, R7 and R8 are as defined in the claims, or a group of structure:

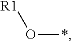

wherein:
* indicates the point of attachment of said group with the rest of the molecule, and
R1 is defined herein;
   or a stereoisomer, a tautomer, an N-oxide, a hydrate, a solvate, or a salt thereof, or a mixture of same, as described and defined herein, and as hereinafter referred to as “compounds of the present invention”, or their pharmacological activity.

It has now been found, and this constitutes the basis of the present invention, that said compounds of the present invention have surprising and advantageous properties.

In particular, said compounds of the present invention have surprisingly been found to effectively inhibit MKNK-1 kinase and may therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1 kinase, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The state of the art described above does not suggest that the specific substituted imidazopyridazine compounds of general formula (I) of the present invention as defined herein would be so active as inhibitors of MKNK-1 kinase.

### DESCRIPTION OF THE INVENTION

In accordance with a first aspect, the present invention covers compounds of general formula (I):

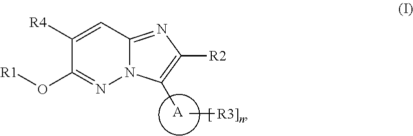

selected from:

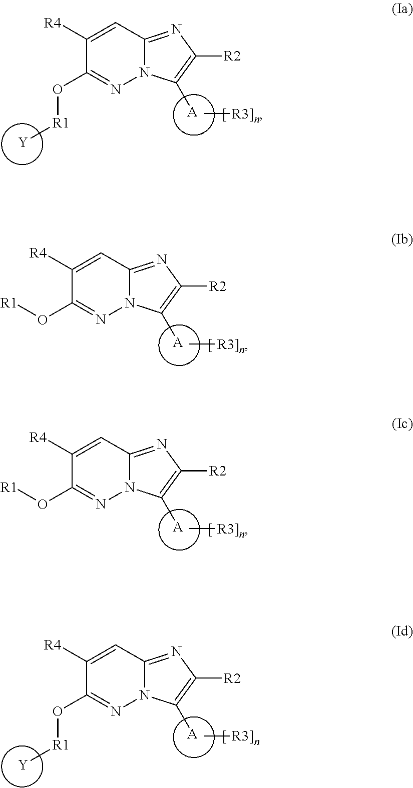

In accordance with a first variant of the first aspect, the present invention covers compounds of general formula (Ia):

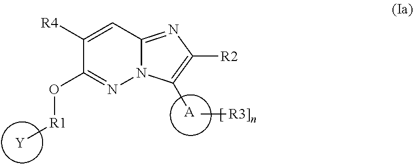

in which:

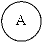

represents a:

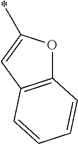 [SMILES: *c1cc2ccccc2o1]

group; wherein * indicates the point of attachment of said group with the rest of the molecule; and

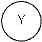

represents a:

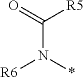

group, or a

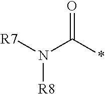

group; wherein * indicates the point of attachment of said groups to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R2 represents a hydrogen atom;
R3 represents a substituent selected from:
a halogen atom, a —CN, C₁-C₆-alkyl-, C₁-C₆-haloalkyl-, C₂-C₆-alkenyl-, C₂-C₆-alkynyl-, —C(═O)R′, —C(═O)NH₂, —C(═O)N(H)R′, —C(═O)N(R′)R″, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₃-C₆-cycloalkyl-C₁-C₆-alkoxy-, C₁-C₆-haloalkoxy-, —OC(═O)R′, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R4 represents a substituent selected from:
a hydrogen atom, a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
   a hydrogen atom, a C₁-C₆-alkyl-, C₃-C₁₀-cycloalkyl-, C₁-C₆-haloalkyl group;
      or:
together, represent a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8 together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0, 1, 2, 3, 4 or 5;
   or a stereoisomer, a tautomer, an N-oxide, a hydrate, a solvate, or a salt thereof, or a mixture of same.

In accordance with a second variant of the first aspect, the present invention covers compounds of general formula (Ib):

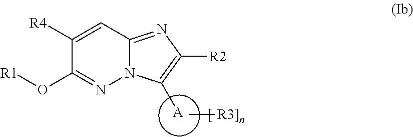

in which:

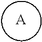

represents a:

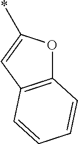 [SMILES: *c1cc2ccccc2o1]

group; wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom, a halogen atom, a —CN, C₁-C₆-alkyl-, C₁-C₆-haloalkyl-, C₃-C₁₀-cycloalkyl-, 3- to 10-membered heterocycloalkyl- group;
R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1, 2, 3, 4 or 5;
or a stereoisomer, a tautomer, an N-oxide, a hydrate, a solvate, or a salt thereof, or a mixture of same.

In accordance with a third variant of the first aspect, the present invention covers compounds of general formula (Ic):

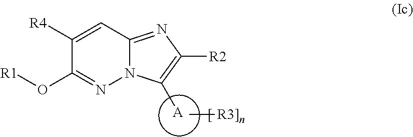

in which:

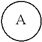

represents a group selected from:

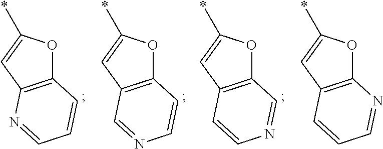

group; wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom, a halogen atom, a —CN, C₁-C₆-alkyl-, C₁-C₆-haloalkyl-, C₃-C₁₀-cycloalkyl-, 3- to 10-membered heterocycloalkyl group;
R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1, 2, 3 or 4;
or a stereoisomer, a tautomer, an N-oxide, a hydrate, a solvate, or a salt thereof, or a mixture of same.

In accordance with a fourth variant of the first aspect, the present invention covers compounds of general formula (Id):

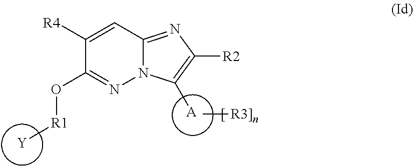

in which:

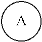

represents a group selected from:

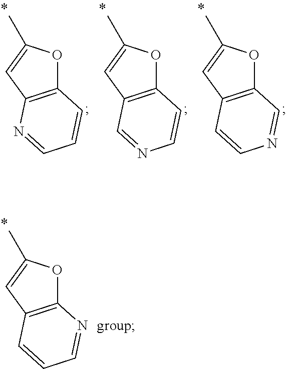

wherein * indicates the point of attachment of said group with the rest of the molecule; and

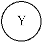

represents a:

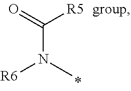

or a

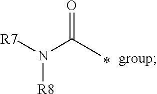

wherein * indicates the point of attachment of said group to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R2 represents a hydrogen atom;
R3 represents a substituent selected from:
a halogen atom, a —CN, C₁-C₆-alkyl-, C₁-C₆-haloalkyl-, C₂-C₆-alkenyl-, C₂-C₆-alkynyl-, —C(═O)R′, —C(═O)NH₂, —C(═O)N(H)R′, —C(═O)N(R′)R″, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₃-C₆-cycloalkyl-C₁-C₆-alkoxy-, C₁-C₆-haloalkoxy-, —OC(═O)R′, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R4 represents a substituent selected from:
a hydrogen atom, a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8, together with a carbon atom of R1, represent a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0, 1, 2, 3 or 4;
   or a stereoisomer, a tautomer, an N-oxide, a hydrate, a solvate, or a salt thereof, or a mixture of same.

For the compounds of general formulae (Ia) and (Id) the terms as mentioned in the present text have the following meanings:

The term “halogen atom”, “halo-” or “Hal-” is to be understood as meaning a fluorine, chlorine, bromine or iodine atom, preferably a fluorine, chlorine, bromine or iodine atom.

The term “C₁-C₆-alkyl” is to be understood as meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “C₁-C₆-haloalkyl” is to be understood as meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said C₁-C₆-haloalkyl group is, for example, —CF₃, —CHF₂, —CH₂F, —CF₂CF₃, or —CH₂CF₃.

The term “C₁-C₆-hydroxyalkyl is to be understood as meaning a linear or branched, saturated, monovalent hydrocarbon group in which the term “C₁-C₆-alkyl” is defined supra, and in which one or more hydrogens atom is replaced by a hydroxy group. Particularly, said “C₁-C₆-hydroxyalkyl” can contain 1, 2 or 3 carbon atoms, (a “C₁-C₃-hydroxyalkyl”), e.g. a —CH₂OH, —CH₂CH₂OH, —CH(OH)CH₃, —CH₂CH₂CH₂OH, or —C(CH₃)₂OH group.

The term “C₁-C₆-alkoxy” is to be understood as meaning a linear or branched, saturated, monovalent, hydrocarbon group of formula —O-alkyl, in which the term “alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof. Particularly, said “C₁-C₆-alkoxy” can contain 1, 2, 3, 4 or 5 carbon atoms, (a “C₁-C₅-alkoxy”).

The term “C₁-C₆- haloalkoxy” is to be understood as meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said C₁-C₆-haloalkoxy group is, for example, —OCF₃, —OCHF₂, —OCH₂F, —OCF₂CF₃, or —OCH₂CF₃.

The term “C₁-C₆-alkoxy-C₁-C₆-alkyl” is to be understood as meaning a linear or branched, saturated, monovalent alkyl group, as defined supra, in which one or more of the hydrogen atoms is replaced, in identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, in which the term “C₁-C₆-alkyl” is defined supra, or an isomer thereof.

The term “C₁-C₆-haloalkoxy-C₁-C₆-alkyl” is to be understood as meaning a linear or branched, saturated, monovalent C₁-C₆-alkoxy-C₁-C₆-alkyl group, as defined supra, in which one or more of the hydrogen atoms is replaced, identically or differently, by a halogen atom. Particularly, said halogen atom is F. Said C₁-C₆-haloalkoxy-C₁-C₆-alkyl group is, for example, —CH₂CH₂OCF₃, —CH₂CH₂OCHF₂, —CH₂CH₂OCH₂F, —CH₂CH₂OCF₂CF₃, or —CH₂CH₂OCH₂CF₃.

The term “C₂-C₆-alkenyl” is to be understood as meaning a linear or branched, monovalent hydrocarbon group, which contains one or more double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₂-C₆-alkynyl” is to be understood as meaning a linear or branched, monovalent hydrocarbon group which contains one or more triple bonds, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-inyl, hex-3-inyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-inyl, 1,1-dimethylbut-3-ynyl, 1,1-dimethylbut-2-ynyl, or 3,3-dimethylbut-1-ynyl group. Particularly, said alkynyl group is ethynyl, prop-1-ynyl, or prop-2-inyl.

The term “C₃-C₁₀-cycloalkyl” is to be understood as meaning a saturated, monovalent, mono-, or bicyclic hydrocarbon ring which contains 3, 4, 5, 6, 7, 8, 9 or 10 carbon atoms (“C₃-C₁₀-cycloalkyl”). Said C₃-C₁₀-cycloalkyl group is for example, a monocyclic hydrocarbon ring, e.g. a cyclopropyl, cyclobutyl, cyclopentyl, cyclohexyl, cycloheptyl, cyclooctyl, cyclononyl or cyclodecyl, or a bicyclic hydrocarbon ring, e.g. a perhydropentalenylene or decalin ring. Particularly, said ring contains 3, 4, 5 or 6 carbon atoms (“C₃-C₆-cycloalkyl”).

The term “C₃-C₆-cycloalkoxy” is to be understood as meaning a saturated, monovalent, hydrocarbon ring which contains 3, 4, 5 or 6 carbon atoms of formula —O-cycloalkyl, in which the term “cycloalkyl” is defined supra, e.g. a cyclopropyloxy, cyclobutyloxy, cyclopentyloxy or cyclohexyloxy.

The term “C₃-C₆-cycloalkyl-C₁-C₆-alkyl” is to be understood as meaning a saturated, monovalent alkyl group, as defined supra, in which one of the hydrogen atoms is replaced by a C₃-C₆-cycloalkyl group, as defined supra, e.g. cyclopropylalkyl, cyclobutylalkyl, cyclopentylalkyl, cyclohexylalkyl group, in which the term “alkyl” is defined supra, or an isomer thereof.

The term “C₃-C₆-cycloalkyl-C₁-C₆-alkoxy” is to be understood as meaning a saturated, monovalent alkoxy group, as defined supra, in which one of the hydrogen atoms is replaced by a C₃-C₆-cycloalkyl group, as defined supra, e.g. cyclopropylalkoxy, cyclobutylalkoxy, cyclopentylalkoxy, cyclohexylalkoxy group, in which the term “alkoxy” is defined supra, or an isomer thereof.

The term “C₄-C₁₀-cycloalkenyl” is to be understood as meaning a monovalent, mono-, or bicyclic hydrocarbon ring which contains 4, 5, 6, 7, 8, 9 or 10 carbon atoms and one, two, three or four double bonds, in conjugation or not, as the size of said cycloalkenyl ring allows. Said C₄-C₁₀-cycloalkenyl group is for example, a monocyclic hydrocarbon ring, e.g. a cyclobutenyl, cyclopentenyl, or cyclohexenyl or a bicyclic hydrocarbon, e.g.:

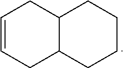

The term “3- to 10-membered heterocycloalkyl”, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom, or a C₁-C₆-alkyl- or C₁-C₆-haloalkyl- group; it being possible for said heterocycloalkyl group to be attached to the rest of the molecule via any one of the carbon atoms or, if present, the nitrogen atom.

Particularly, said 3- to 10-membered heterocycloalkyl can contain 2, 3, 4, or 5 carbon atoms, and one or more of the above-mentioned heteroatom-containing groups (a “3- to 6-membered heterocycloalkyl”), more particularly said heterocycloalkyl can contain 4 or 5 carbon atoms, and one or more of the above-mentioned heteroatom-containing groups (a “5- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, pyrrolinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example. Optionally, said heterocycloalkyl can be benzo fused.

Said heterocyclyl can be bicyclic, such as, without being limited thereto, a 5,5-membered ring, e.g. a hexahydrocyclopenta[c]pyrrol-2(1H)-yl ring, or a 5,6-membered bicyclic ring, e.g. a hexahydropyrrolo[1,2-a]pyrazin-2(1H)-yl ring.

As mentioned supra, said nitrogen atom-containing ring can be partially unsaturated, i.e. it can contain one or more double bonds, such as, without being limited thereto, a 2,5-dihydro-1H-pyrrolyl, 4H-[1,3,4]thiadiazinyl, 4,5-dihydrooxazolyl, or 4H-[1,4]thiazinyl ring, for example, or, it may be benzo-fused, such as, without being limited thereto, a dihydroisoquinolinyl ring, for example.

The term “4- to 10-membered heterocycloalkenyl”, is to be understood as meaning an unsaturated, monovalent, mono- or bicyclic hydrocarbon ring which contains 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom, or a C₁-C₆-alkyl- or C₁-C₆-haloalkyl- group; it being possible for said heterocycloalkenyl group to be attached to the rest of the molecule via any one of the carbon atoms or, if present, the nitrogen atom. Examples of said heterocycloalkenyl may contain one or more double bonds, e.g. 4H-pyranyl, 2H-pyranyl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl group, or, it may be benzo fused.

The term “aryl” is to be understood as meaning a monovalent, aromatic or partially aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a biphenyl group, or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a ring having 13 carbon atoms, (a “C₁₃-aryl” group), e.g. a fluorenyl group, or a ring having 14 carbon atoms, (a “C₁₄-aryl” group), e.g. an anthranyl group.

The term “aryl-C₁-C₆-alkyl” is to be understood as meaning a saturated, monovalent alkyl group, as defined supra, in which one of the hydrogen atoms is replaced by an aryl group, as defined supra.

The term “heteroaryl” is understood as meaning a monovalent, monocyclic-, bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

For the compounds of general formulae (Ib) and (Ic) the terms as mentioned in the present text have the following meanings:

The term “halogen atom”, “halo-” or “Hal-” is to be understood as meaning a fluorine, chlorine, bromine or iodine atom, preferably a fluorine, chlorine, bromine or iodine atom.

The term “C1-C6-alkyl” is to be understood as meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C1-C4-alkyl”), e.g. a methyl, ethyl, propyl, butyl, iso-propyl, iso-butyl, sec-butyl, tert-butyl group, more particularly 1, 2 or 3 carbon atoms (“C1-C3-alkyl”), e.g. a methyl, ethyl, n-propyl- or iso-propyl group.

The term “C1-C6-haloalkyl” is to be understood as meaning a linear or branched, saturated, monovalent hydrocarbon group in which the term “C1-C6-alkyl” is defined supra, and in which one or more hydrogen atoms is replaced by a halogen atom, in identically or differently, i.e. one halogen atom being independent from another. Particularly, said halogen atom is F. Said C1-C6-haloalkyl group is, for example, —CF3, —CHF2, —CH2F, —CF2CF3, or —CH2CF3.

The term “C1-C6-hydroxyalkyl” is to be understood as meaning a linear or branched, saturated, monovalent hydrocarbon group in which the term “C1-C6-alkyl” is defined supra, and in which one or more hydrogens atom is replaced by a hydroxy group. Particularly, said “C1-C6-hydroxyalkyl” can contain 1, 2 or 3 carbon atoms, (a “C1-C3-hydroxyalkyl”), e.g. a —CH2OH, —CH2CH2OH, —CH(OH)CH3, —CH2CH2CH2OH, or —C(CH3)₂OH group.

The term “C1-C6-alkoxy” is to be understood as meaning a linear or branched, saturated, monovalent, hydrocarbon group of formula —O-alkyl, in which the term “alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof. Particularly, said “C1-C6-alkoxy” can contain 1, 2, 3, 4 or 5 carbon atoms, (a “C1-C5-alkoxy”).

The term “C1-C6- haloalkoxy” is to be understood as meaning a linear or branched, saturated, monovalent C1-C6-alkoxy group, as defined supra, in which one or more of the hydrogen atoms is replaced, in identically or differently, by a halogen atom. Particularly, said halogen atom is F. Said C1-C6-haloalkoxy group is, for example, —OCF3, —OCHF2, —OCH2F, —OCF2CF3, or —OCH2CF3.

The term “C1-C6-alkoxy-C1-C6-alkyl” is to be understood as meaning a linear or branched, saturated, monovalent alkyl group, as defined supra, in which one or more of the hydrogen atoms is replaced, in identically or differently, by a C1-C6-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, in which the term “C1-C6-alkyl” is defined supra, or an isomer thereof.

The term “C1-C6-haloalkoxy-C1-C6-alkyl” is to be understood as meaning a linear or branched, saturated, monovalent C1-C6-alkoxy-C1-C6-alkyl group, as defined supra, in which one or more of the hydrogen atoms is replaced, identically or differently, by a halogen atom. Particularly, said halogen atom is F. Said C1-C6-haloalkoxy-C1-C6-alkyl group is, for example, —CH2CH2OCF3, —CH2CH2OCHF2, —CH2CH2OCH2F, —CH2CH2OCF2CF3, or —CH2CH2OCH2CF3.

The term “C2-C6-alkenyl” is to be understood as meaning a linear or branched, monovalent hydrocarbon group, which contains one or more double bonds, and which has 2, 3, 4, 5 or 6 carbon atoms, particularly 2 or 3 carbon atoms (“C2-C3-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C2-C6-alkynyl” is to be understood as meaning a linear or branched, monovalent hydrocarbon group which contains one or more triple bonds, and which contains 2, 3, 4, 5 or 6 carbon atoms, particularly 2 or 3 carbon atoms (“C2-C3-alkynyl”). Said C2-C6-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-inyl, hex-3-inyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethyl-but-3-ynyl, 1-ethylbut-2-ynyl, 1-propylprop-2-ynyl, 1-isopropylprop-2-ynyl, 2,2-di-me-thyl-but-3-inyl, 1,1-dimethylbut-3-ynyl, 1,1-dimethylbut-2-ynyl, or 3,3-di-methyl-but-1-ynyl group. Particularly, said alkynyl group is ethynyl, prop-1-ynyl, or prop-2-inyl.

The term “C3-C10-cycloalkyl” is to be understood as meaning a saturated, monovalent, mono-, or bicyclic hydrocarbon ring which contains 3, 4, 5, 6, 7, 8, 9 or 10 carbon atoms (“C3-C10-cycloalkyl”). Said C3-C10-cycloalkyl group is for example, a monocyclic hydrocarbon ring, e.g. a cyclopropyl, cyclobutyl, cyclopentyl, cyclohexyl, cycloheptyl, cyclooctyl, cyclononyl or cyclodecyl, or a bicyclic hydrocarbon ring, e.g. a perhydropentalenylene or decalin ring. Particularly, said ring contains 3, 4, 5 or 6 carbon atoms (“C3-C6-cycloalkyl”). Cycloalkyl rings containing 5, 6, 7, 8, 9 or 10 carbon atoms (“C5-C10-cycloalkyl”) are optionally benzo fused, e.g. indanyl- or 1,2,3,4-tetrahydronaphtalenyl.

The term “C3-C6-cycloalkoxy” is to be understood as meaning a saturated, monovalent, hydrocarbon ring which contains 3, 4, 5 or 6 carbon atoms of formula —O-cycloalkyl, in which the term “cycloalkyl” is defined supra, e.g. a cyclopropyloxy, cyclobutyloxy, cyclopentyloxy or cyclohexyloxy.

The term “C3-C6-cycloalkyl-C1-C6-alkyl” is to be understood as meaning a saturated, monovalent alkyl group, as defined supra, in which one of the hydrogen atoms is replaced by a C3-C6-cycloalkyl group, as defined supra, e.g. cyclopropylalkyl, cyclobutylalkyl, cyclopentylalkyl, cyclohexylalkyl group, in which the term “alkyl” is defined supra, or an isomer thereof.

The term “C3-C6-cycloalkyl-C1-C6-alkoxy” is to be understood as meaning a saturated, monovalent alkoxy group, as defined supra, in which one of the hydrogen atoms is replaced by a C3-C6-cycloalkyl group, as defined supra, e.g. cyclopropylalkoxy, cyclobutylalkoxy, cyclopentylalkoxy, cyclohexylalkoxy group, in which the term “alkoxy” is defined supra, or an isomer thereof.

The term “C4-C10-cycloalkenyl” is to be understood as meaning a monovalent, mono-, or bicyclic hydrocarbon ring which contains 4, 5, 6, 7, 8, 9 or 10 carbon atoms and one, two, three or four double bonds, in conjugation or not, as the size of said cycloalkenyl ring allows. Said C4-C10-cycloalkenyl group is for example, a monocyclic hydrocarbon ring, e.g. a cyclobutenyl, cyclopentenyl, or cyclohexenyl or a bicyclic hydrocarbon, e.g.:

The term “4- to 10-membered heterocycloalkyl”, is to be understood as meaning a saturated, monovalent, mono- or bicyclic hydrocarbon ring which contains 3, 4, 5, 6, 7, 8 or 9 carbon atoms, and one or more heteroatom-containing groups selected from C(═O), O, S, S(═O), S(═O)2, NR^{a}, in which Ra represents a hydrogen atom, or a C1-C6-alkyl-, a C1-C6-hydroxyalkyl-, a C1-C6-haloalkyl-, C1-C6-alkyl-(C═O)— or aryl group; it being possible for said heterocycloalkyl group to be attached to the rest of the molecule via any one of the carbon atoms or, if present, the nitrogen atom.

Particularly, said 4- to 10-membered heterocycloalkyl can contain 3, 4, or 5 carbon atoms, and one or more of the above-mentioned heteroatom-containing groups (a “4- to 6-membered heterocycloalkyl”), more particularly said heterocycloalkyl can contain 4 or 5 carbon atoms, and one or more of the above-mentioned heteroatom-containing groups (a “5- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, pyrrolinyl, or oxopyrrolidinyl, or a 6-membered ring, such as tetrahydropyranyl, piperidinyl, morpholinyl, dithianyl, thiomorpholinyl, piperazinyl, trithianyl, oxopiperidinyl, oxopiperazinyl, or oxomorpholinyl, or a 7-membered ring, such as a diazepanyl ring, for example. Optionally, said heterocycloalkyl can be benzo fused.

Said heterocyclalkyl can be bicyclic, such as, without being limited thereto, a 5,5-membered ring, e.g. a hexahydrocyclopenta[c]pyrrol-2(1H)-yl ring, or a 5,6-membered bicyclic ring, e.g. a hexahydropyrrolo[1,2-a]pyrazin-2(1H)-yl ring.

As mentioned supra, said nitrogen atom-containing ring can be partially unsaturated, i.e. it can contain one or more double bonds, such as, without being limited thereto, a 2,5-dihydro-1H-pyrrolyl, 4H-[1,3,4]thiadiazinyl, 4,5-dihydrooxazolyl, or 4H-[1,4]thiazinyl ring, for example, or, it may be benzo-fused, such as, without being limited thereto, a dihydroisoquinolinyl ring, for example.

The term “4- to 10-membered nitrogen atom containing heterocycloalkyl group”, is to be understood as meaning a saturated, monovalent, mono- or bicyclic ring which contains 3, 4, 5, 6, 7, 8 or 9 carbon atoms and at least one nitrogen atom, optionally containing more heteroatom-containing groups selected from C(═O), O, S, S(═O), S(═O)2, NR^{a}, in which Ra represents a hydrogen atom, or a C1-C6-alkyl-, a C1-C6-hydroxyalkyl-, a C1-C6-haloalkyl-, C1-C6-alkyl-(C═O)— or aryl group; said nitrogen atom containing heterocycloalkyl group being attached to the rest of the molecule via a nitrogen atom, which is a ring atom.

Particularly, without being limited thereto, said nitrogen atom containing heterocycloalkyl can be a 4-membered ring, such as an azetidinyl, or a 5-membered ring, such as a pyrrolidinyl, imidazolidinyl, pyrazolidinyl, pyrrolinyl, or oxopyrrolidinyl, or a 6-membered ring, such as piperidinyl, morpholinyl, thiomorpholinyl, piperazinyl, oxopiperidinyl, oxopiperazinyl, or oxomorpholinyl, or a 7-membered ring, such as a diazepanyl ring, for example.

The term “4- to 10-membered heterocycloalkenyl”, is to be understood as meaning an unsaturated, monovalent, mono- or bicyclic hydrocarbon ring which contains 3, 4, 5, 6, 7, 8 or 9 carbon atoms, and one or more heteroatom-containing groups selected from C(═O), O, S, S(═O), S(═O)2, NR^{a}, in which Ra represents a hydrogen atom, or a C1-C6-alkyl- or C1-C6-haloalkyl- group; it being possible for said heterocycloalkenyl group to be attached to the rest of the molecule via any one of the carbon atoms or, if present, the nitrogen atom. Examples of said heterocycloalkenyl may contain one or more double bonds, e.g. 4H-pyranyl, 2H-pyranyl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl group, or, it may be benzo fused.

The term “aryl” is to be understood as meaning a monovalent, aromatic or partially aromatic, mono-, or bi- or tricyclic hydrocarbon ring having 6, 7, 8, 9, 10, 11, 12, 13 or 14 carbon atoms (a “C6-C14-aryl” group), particularly a ring having 6 carbon atoms (a “C6-aryl” group), e.g. a phenyl group; or a biphenyl group, or a ring having 9 carbon atoms (a “C9-aryl” group), e.g. an indanyl or indenyl group, or a ring having 10 carbon atoms (a “C10-aryl” group), e.g. a tetralinyl, dihydronaphthyl, or naphthyl group, or a ring having 13 carbon atoms, (a “C13-aryl” group), e.g. a fluorenyl group, or a ring having 14 carbon atoms, (a “C14-aryl” group), e.g. an anthranyl group.

The term “aryl-C1-C6-alkyl” is to be understood as meaning a saturated, monovalent alkyl group, as defined supra, in which one of the hydrogen atoms is replaced by an aryl group, as defined supra.

The term “heteroaryl” is understood as meaning a monovalent, monocyclic-, bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

For the compounds of general formulae (Ia), (Ib), (Ic) and (Id) the terms as mentioned in the present text have the following meanings:

In general, and unless otherwise mentioned, the heteroarylic or heteroarylenic radicals include all the possible isomeric forms thereof, e.g. the positional isomers thereof. Thus, for some illustrative non-restricting example, the term pyridinyl or pyridinylene includes pyridin-2-yl, pyridin-2-ylene, pyridin-3-yl, pyridin-3-ylene, pyridin-4-yl and pyridin-4-ylene; or the term thienyl or thienylene includes thien-2-yl, thien-2-ylene, thien-3-yl and thien-3-ylene.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C1-C5; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆. The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of this invention may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention may contain sulphur atoms which are asymmetric, such as an asymmetric sulphoxide or sulphoximine group, of structure:

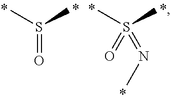

for example,
in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

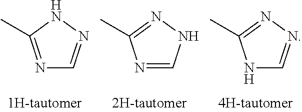

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

As used herein, the term “in vivo hydrolysable ester” is understood as meaning an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorphs, in any ratio.

In accordance with a second embodiment of the first variant of the first aspect, the present invention covers compounds of general formula (Ia), supra, in which:

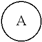

represents a:

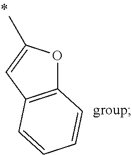

wherein * indicates the point of attachment of said group with the rest of the molecule; and

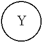

represents a:

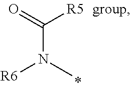

or a ;

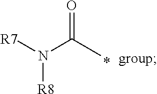

wherein * indicates the point of attachment of said groups to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R2 represents a hydrogen atom;
R3 represents a substituent selected from:
a halogen atom, a —CN, C₁-C₆-alkyl-, C₁-C₆-haloalkyl-, C₂-C₆-alkenyl-, C₂-C₆-alkynyl-, —C(═O)R′, —C(═O)NH₂, —C(═O)N(H)R′, —C(═O)N(R′)R″, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₃-C₆-cycloalkyl-C₁-C₆-alkoxy-, C₁-C₆-haloalkoxy-, —OC(═O)R′, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R4 represents a substituent selected from:
a hydrogen atom, a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent
R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, represent a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8 together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0, 1, 2, 3, 4 or 5;
   or a stereoisomer, a tautomer, an N-oxide, a hydrate, a solvate, or a salt thereof, or a mixture of same.

In accordance with a third embodiment of the first variant of the first aspect, the present invention covers compounds of general formula (Ia), supra, in which:

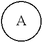

represents a:

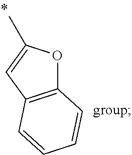

wherein * indicates the point of attachment of said group with the rest of the molecule; and

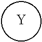

represents a:

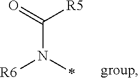

or a

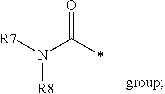

wherein * indicates the point of attachment of said groups to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R2 represents a hydrogen atom;
R3 represents a substituent selected from:
a halogen atom, a —CN, C₁-C₆-alkyl-, C₁-C₆-haloalkyl-, —NHR′, —OH, C₁-C₆-alkoxy-, C₃-C₆-cycloalkyl-C₁-C₃-alkoxy-, C₁-C₆-haloalkoxy-
R4 represents a substituent selected from:
a hydrogen atom, a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent
R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, represent a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8 together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0 or 1;
   or a stereoisomer, a tautomer, an N-oxide, a hydrate, a solvate, or a salt thereof, or a mixture of same.

In accordance with a fourth embodiment of the first variant of the first aspect, the present invention covers compounds of general formula (Ia), supra, in which:

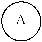

represents a:

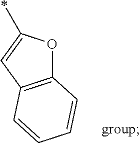 [SMILES: *c1cc2ccccc2o1]

wherein * indicates the point of attachment of said group with the rest of the molecule; and

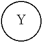

represents a:

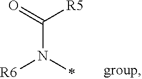

or a

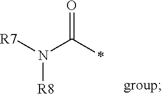

wherein * indicates the point of attachment of said groups to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—;
R2 represents a hydrogen atom;
R3 represents a substituent selected from:
a halogen atom, a —CN, C₁-C₆-alkyl-, C₁-C₆-haloalkyl-, —NHR′, —OH, C₁-C₆-alkoxy-, C₃-C₆-cycloalkyl-C₁-C₃-alkoxy-, C₁-C₆-haloalkoxy-
R4 represents a substituent selected from:
a hydrogen atom, a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent
R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, represent a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8 together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0 or 1;
   or a stereoisomer, a tautomer, an N-oxide, a hydrate, a solvate, or a salt thereof, or a mixture of same.

In accordance with a fifth embodiment of the first variant of the first aspect, the present invention covers compounds of general formula (Ia), supra, in which:

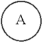

represents a:

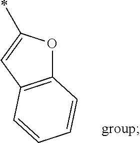 [SMILES: *c1cc2ccccc2o1]

wherein * indicates the point of attachment of said group with the rest of the molecule; and

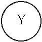

represents a:

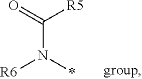

or a

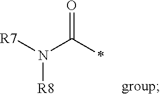

wherein * indicates the point of attachment of said groups to R1; and
R1 represents a linear C₁-C₆-alkyl-, a branched C₃-C₆-alkyl-, or a C₃-C₆-cycloalkyl group which is optionally substituted with a heteroaryl-group;
R2 represents a hydrogen atom;
R3 represents a substituent selected from:
a halogen atom or a C₁-C₆-alkoxy-group;
R4 represents a hydrogen atom;
R5 represents:
either:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted with a methyl- or chloro-group; heteroaryl- optionally substituted with a methyl-group;
   or:
together with a carbon atom of R1, represents a 5- or 6-membered cyclic amide group;
said 5- or 6-membered cyclic amide group optionally containing one further heteroatom selected from the group consisting of O and N;
   R6 represents:
   either:
a substituent selected from hydrogen or a C₁-C₆-alkyl-group;
   or:
together with a carbon atom of R1, represents a 5- or 6-membered cyclic amine group;
said 6-membered cyclic amine group optionally containing one further heteroatom consisting of O;
   or:
R5 and R6 together represent a 5-membered cyclic amide group:
said 5-membered cyclic amide group optionally containing one further heteroatom consisting of N;
   R7 and R8 represent:
   either:
independently from each other, a substituent selected from:
a hydrogen atom or a C₁-C₆-alkyl-group;
   or:
R7 or R8 together with a carbon atom of R1, represents a 5-membered cyclic amide group:
   R represents a substituent selected from:
   a halogen atom, a —CN, C₁-C₆-alkyl-, C₁-C₆-haloalkyl-group;
   R′ and R″ represent, independently from each other, a substituent selected from:
   a C₁-C₆-alkyl-group;
   n represents an integer of 0 or 1;
   or a stereoisomer, a tautomer, an N-oxide, a hydrate, a solvate, or a salt thereof, or a mixture of same.

In a further embodiment of the above-mentioned first variant of the first aspect, the invention relates to compounds of formula (Ia), wherein:

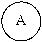

represents a:

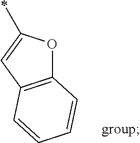 [SMILES: *c1cc2ccccc2o1]

wherein * indicates the point of attachment of said group with the rest of the molecule.

In a further embodiment of the above-mentioned first variant of the first aspect, the invention relates to compounds of formula (Ia), wherein:

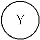

represents a:

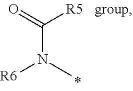

or a

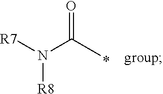

wherein * indicates the point of attachment of said groups to R1.

In a further embodiment of the above-mentioned first variant of the first aspect, the invention relates to compounds of formula (Ia), wherein:

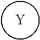

represents a:

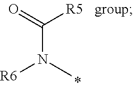

wherein * indicates the point of attachment of said groups to R1.

In a further embodiment of the above-mentioned first variant of the first aspect, the invention relates to compounds of formula (Ia), wherein:

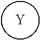

represents a:

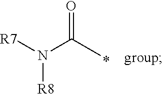

wherein * indicates the point of attachment of said groups to R1.

In a further embodiment of the above-mentioned first variant of the first aspect, the invention relates to compounds of formula (Ia), wherein:

R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group.

In a further embodiment of the above-mentioned first variant of the first aspect, the invention relates to compounds of formula (Ia), wherein:

R1 represents a linear C₁-C₆-alkyl-, a branched C₃-C₆-alkyl-, or a C₃-C₆-cycloalkyl group.

In a further embodiment of the above-mentioned first variant of the first aspect, the invention relates to compounds of formula (Ia), wherein:

R2 represents a hydrogen atom.

In a further embodiment of the above-mentioned first variant of the first aspect, the invention relates to compounds of formula (Ia), wherein:

R3 represents a substituent selected from:
a halogen atom, a —CN, C₁-C₆-alkyl-, C₁-C₆-haloalkyl-, C₂-C₆-alkenyl-, C₂-C₆-alkynyl-, —C(═O)R′, —C(═O)NH₂, —C(═O)N(H)R′, —C(═O)N(R′)R″, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₃-C₆-cycloalkyl-C₁-C₆-alkoxy-, C₁-C₆-haloalkoxy-, —OC(═O)R′, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group.

In a further embodiment of the above-mentioned first variant of the first aspect, the invention relates to compounds of formula (Ia), wherein:

R4 represents a substituent selected from:
a hydrogen atom, a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group.

In a further embodiment of the above-mentioned first variant of the first aspect, the invention relates to compounds of formula (Ia), wherein:

R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl- optionally substituted one or more times, independently from each other, with an R substituent group
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned first variant of the first aspect, the invention relates to compounds of formula (Ia), wherein:

R5 represents:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl- optionally substituted one or more times, independently from each other, with an R substituent group.

In a further embodiment of the above-mentioned first variant of the first aspect, the invention relates to compounds of formula (Ia), wherein:

### R5

together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned first variant of the first aspect, the invention relates to compounds of formula (Ia), wherein:

R6 represents:
either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned first variant of the first aspect, the invention relates to compounds of formula (Ia), wherein:

R6 represents:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent group.

In a further embodiment of the above-mentioned first variant of the first aspect, the invention relates to compounds of formula (Ia), wherein:

### R6

together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S.

In a further embodiment of the above-mentioned first variant of the first aspect, the invention relates to compounds of formula (Ia), wherein:

### R6

together with R5 forms a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned first variant of the first aspect, the invention relates to compounds of formula (Ia), wherein:

R7 and R8 represent:
either:
independently from each other, a substituent selected from:
a hydrogen atom, a C₁-C₆-alkyl-, C₃-C₁₀-cycloalkyl-, C₁-C₆-haloalkyl group;
   or:
together, represent a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8 together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned first variant of the first aspect, the invention relates to compounds of formula (Ia), wherein:

R7 and R8 represent:
independently from each other, a substituent selected from:
a hydrogen atom, a C₁-C₆-alkyl-, C₃-C₁₀-cycloalkyl-, C₁-C₆-haloalkyl group.

In a further embodiment of the above-mentioned first variant of the first aspect, the invention relates to compounds of formula (Ia), wherein:

### R7 and R8

together, represent a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S.

In a further embodiment of the above-mentioned first variant of the first aspect, the invention relates to compounds of formula (Ia), wherein:

### R7 or R8

together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned first variant of the first aspect, the invention relates to compounds of formula (Ia), wherein:

R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group.

In a further embodiment of the above-mentioned first variant of the first aspect, the invention relates to compounds of formula (Ia), wherein:

R′ and R″ represent, independently from each other, a substituent selected from:
a C₁-C₆-alkyl-, C₃-C₁₀-cycloalkyl-, C₁-C₆-haloalkyl group.

In a further embodiment of the above-mentioned first variant of the first aspect, the invention relates to compounds of formula (Ia), wherein:

n represents an integer of 0, 1, 2, 3, 4 or 5.

In a further embodiment of the above-mentioned first variant of the first aspect, the invention relates to compounds of formula (Ia), wherein:

R4 represents a substituent selected from:
a hydrogen atom, a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent group.

In a further embodiment of the above-mentioned first variant of the first aspect, the invention relates to compounds of formula (Ia), wherein:

R3 represents a substituent selected from:
a halogen atom, a —CN, C₁-C₆-alkyl-, C₁-C₆-haloalkyl-, —NHR′, —OH, C₁-C₆-alkoxy-, C₃-C₆-cycloalkyl-C₁-C₃-alkoxy-, C₁-C₆-haloalkoxy group.

In a further embodiment of the above-mentioned first variant of the first aspect, the invention relates to compounds of formula (Ia), wherein:

n represents an integer of 0 or 1.

In a further embodiment of the above-mentioned first variant of the first aspect, the invention relates to compounds of formula (Ia), wherein:

R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group.

In a further embodiment of the above-mentioned first variant of the first aspect, the invention relates to compounds of formula (Ia), wherein:

R1 represents a linear C₁-C₆-alkyl-, a branched C₃-C₆-alkyl-, or a C₃-C₆-cycloalkyl group which is optionally substituted with a heteroaryl-group.

In a further embodiment of the above-mentioned first variant of the first aspect, the invention relates to compounds of formula (Ia), wherein:

R3 represents a substituent selected from:
a halogen atom or a C₁-C₆-alkoxy-group.

In a further embodiment of the above-mentioned first variant of the first aspect, the invention relates to compounds of formula (Ia), wherein:

R5 represents:
either:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted with a methyl- or chloro-group; heteroaryl- optionally substituted with a methyl-group group
   or:
together with a carbon atom of R1, represents a 5- or 6-membered cyclic amide group;
said 5- or 6-membered cyclic amide group optionally containing one further heteroatom selected from the group consisting of O and N.

In a further embodiment of the above-mentioned first variant of the first aspect, the invention relates to compounds of formula (Ia), wherein:

R5 represents:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted with a methyl- or chloro-group; heteroaryl- optionally substituted with a methyl-group group.

In a further embodiment of the above-mentioned first variant of the first aspect, the invention relates to compounds of formula (Ia), wherein:

### R5

together with a carbon atom of R1, represents a 5- or 6-membered cyclic amide group;
said 5- or 6-membered cyclic amide group optionally containing one further heteroatom selected from the group consisting of O and N.

In a further embodiment of the above-mentioned first variant of the first aspect, the invention relates to compounds of formula (Ia), wherein:

R6 represents:
either:
a substituent selected from hydrogen or a C₁-C₆-alkyl-group;
   or:
together with a carbon atom of R1, represents a 5- or 6-membered cyclic amine group;
said 6-membered cyclic amine group optionally containing one further heteroatom consisting of O;
   or:
R5 and R6 together represent a 5-membered cyclic amide group:
said 5-membered cyclic amide group optionally containing one further heteroatom consisting of N.

In a further embodiment of the above-mentioned first variant of the first aspect, the invention relates to compounds of formula (Ia), wherein:

R6 represents:
a substituent selected from hydrogen or a C₁-C₆-alkyl-group.

In a further embodiment of the above-mentioned first variant of the first aspect, the invention relates to compounds of formula (Ia), wherein:

### R6

together with a carbon atom of R1, represents a 5- or 6-membered cyclic amine group;
said 6-membered cyclic amine group optionally containing one further heteroatom consisting of O.

In a further embodiment of the above-mentioned first variant of the first aspect, the invention relates to compounds of formula (Ia), wherein:

### R6

together with R5 forms a 5-membered cyclic amide group:
said 5-membered cyclic amide group optionally containing one further heteroatom consisting of N.

In a further embodiment of the above-mentioned first variant of the first aspect, the invention relates to compounds of formula (Ia), wherein:

R7 and R8 represent:
either:
independently from each other, a substituent selected from:
a hydrogen atom or a C₁-C₆-alkyl-group;
   or:
R7 or R8 together with a carbon atom of R1, represents a 5-membered cyclic amide group.

In a further embodiment of the above-mentioned first variant of the first aspect, the invention relates to compounds of formula (Ia), wherein:

R7 and R8 represent:
independently from each other, a substituent selected from:
a hydrogen atom or a C₁-C₆-alkyl-group.

In a further embodiment of the above-mentioned first variant of the first aspect, the invention relates to compounds of formula (Ia), wherein:

### R7 or R8

together with a carbon atom of R1, represents a 5-membered cyclic amide group.

In a further embodiment of the above-mentioned first variant of the first aspect, the invention relates to compounds of formula (Ia), wherein:

R represents a substituent selected from:
a halogen atom, a —CN, C₁-C₆-alkyl-, C₁-C₆-haloalkyl-group.

In a further embodiment of the above-mentioned first variant of the first aspect, the invention relates to compounds of formula (Ia), wherein:

R′ and R″ represent, independently from each other, a C₁-C₆-alkyl-group.

In a further embodiment of the above-mentioned first variant of the first aspect, the invention relates to compounds of formula (Ia), wherein:

n represents an integer of 0.

In a further embodiment of the above-mentioned first variant of the first aspect, the invention relates to compounds of formula (Ia), wherein:

n represents an integer of 1.

In a further embodiment of the above-mentioned first variant of the first aspect, the invention relates to compounds of formula (Ia), according to any of the above-mentioned embodiments, in the form of or a stereoisomer, a tautomer, an N-oxide, a hydrate, a solvate, or a salt thereof, or a mixture of same.

It is to be understood that the present invention relates to any sub-combination within any embodiment or aspect of the present invention of compounds of general formula (Ia), supra.

More particularly still, the present invention covers compounds of general formula (Ia) which are disclosed in the Example section of this text, infra.

In accordance with another aspect, the present invention covers methods of preparing compounds of the present invention, said methods comprising the steps as described in the Experimental Section herein.

In accordance with a further aspect, the present invention covers intermediate compounds which are useful in the preparation of compounds of the present invention of general formula (Ia), particularly in the method described herein. In particular, the present invention covers compounds of general formula (Ea):

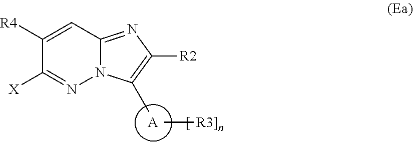

in which A, R2, R3, R4 and n are as defined for the compound of general formula (Ia) supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Ia), particularly in the method described herein. In particular, the present invention covers compounds of general formula (Fa):

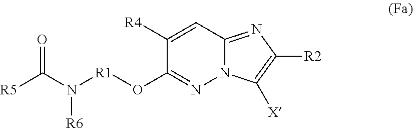

in which R1, R2, R4, R5 and R6 are as defined for the compound of general formula (Ia) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Ia), particularly in the method described herein. In particular, the present invention covers compounds of general formula (Ga):

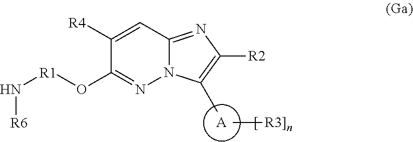

in which A, R1, R2, R3, R4, R6 and n are as defined for the compound of general formula (Ia) supra.

In accordance with a further aspect, the present invention covers intermediate compounds which are useful in the preparation of compounds of the present invention of general formula (Ia), particularly in the method described herein. In particular, the present invention covers compounds of general formula (Ha):

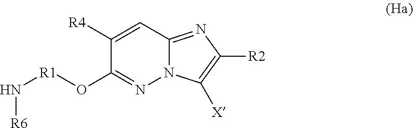

in which R1, R2, R4, and R6 are as defined for the compound of general formula (Ia) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Ia), particularly in the method described herein. In particular, the present invention covers compounds of general formula (Ka):

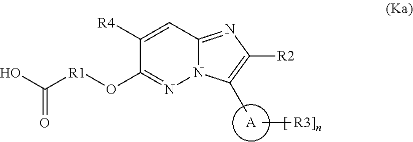

in which A, R1, R2, R3, R4 and n are as defined for the compound of general formula (Ia) supra.

In accordance with a further aspect, the present invention covers intermediate compounds which are useful in the preparation of compounds of the present invention of general formula (Ia), particularly in the method described herein. In particular, the present invention covers compounds of general formula (La):

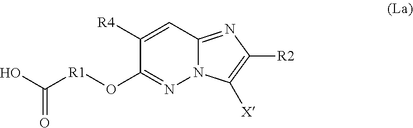

in which R1, R2 and R4 are as defined for the compound of general formula (Ia) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Ia), particularly in the method described herein. In particular, the present invention covers compounds of general formula (Ma):

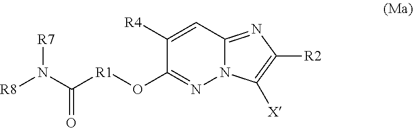

in which R1, R2, R4, R7 and R8 are as defined for the compound of general formula (Ia) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with yet another aspect, the present invention covers the use of the intermediate compounds of general formula (Ea):

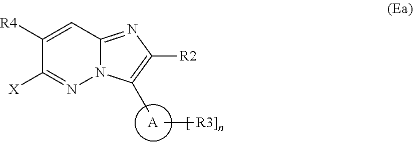

in which A, R2, R3, R4 and n are as defined for the compound of general formula (Ia) supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group for example, for the preparation of a compound of general formula (Ia) as defined supra.

In accordance with yet another aspect, the present invention covers the use of the intermediate compounds of general formula (Fa):

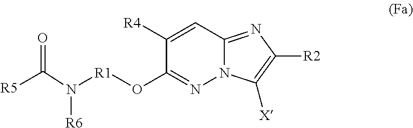

in which R1, R2, R4, R5 and R6 are as defined for the compound of general formula (Ia) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Ia) as defined supra.

In accordance with yet another aspect, the present invention covers the use of the intermediate compounds of general formula (Ga):

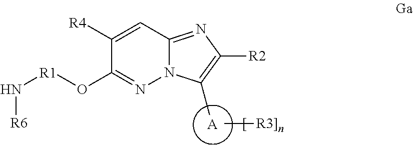

in which A, R1, R2, R3, R4, R6 and n are as defined for the compound of general formula (Ia) supra, for the preparation of a compound of general formula (Ia) as defined supra.

In accordance with yet another aspect, the present invention covers the use of the intermediate compounds of general formula (Ha):

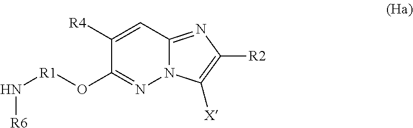

in which R1, R2, R4, and R6 are as defined for the compound of general formula (Ia) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Ia) as defined supra.

In accordance with yet another aspect, the present invention covers the use of the intermediate compounds of general formula (Ka):

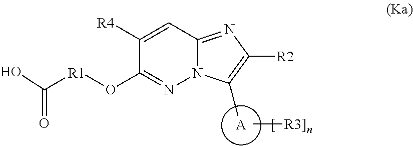

in which A, R1, R2, R3, R4 and n are as defined for the compound of general formula (Ia) supra, for the preparation of a compound of general formula (Ia) as defined supra.

In accordance with yet another aspect, the present invention covers the use of the intermediate compounds of general formula (La):

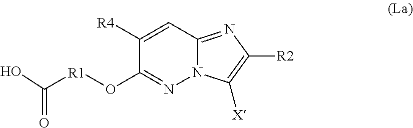

in which R1, R2 and R4 are as defined for the compound of general formula (Ia) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Ia) as defined supra.

In accordance with yet another aspect, the present invention covers the use of the intermediate compounds of general formula (Ma):

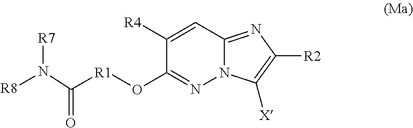

in which R1, R2, R4, R7 and R8 are as defined for the compound of general formula (Ia) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Ia) as defined supra.

In accordance with a second embodiment of the second variant of the first aspect, the present invention covers compounds of general formula (Ib), supra, in which:

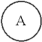

represents a:

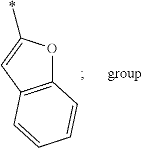 [SMILES: *c1cc2ccccc2o1]

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom, a halogen atom, a —CN, C₁-C₆-alkyl-, C₁-C₆-haloalkyl-, C₃-C₁₀-cycloalkyl-, 3- to 10-membered heterocycloalkyl- group;
R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1;
or a stereoisomer, a tautomer, an N-oxide, a hydrate, a solvate, or a salt thereof, or a mixture of same.

In accordance with a third embodiment of the second variant of the first aspect, the present invention covers compounds of general formula (Ib), supra, in which:

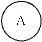

represents a:

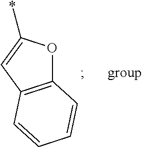 [SMILES: *c1cc2ccccc2o1]

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C1-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1;
or a stereoisomer, a tautomer, an N-oxide, a hydrate, a solvate, or a salt thereof, or a mixture of same.

In accordance with a fourth embodiment of the second variant of the first aspect, the present invention covers compounds of general formula (Ib), supra, in which:

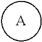

represents a:

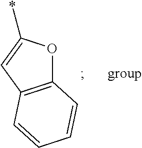 [SMILES: *c1cc2ccccc2o1]

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C1-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1;
or a stereoisomer, a tautomer, an N-oxide, a hydrate, a solvate, or a salt thereof, or a mixture of same.

In accordance with a fifth embodiment of the second variant of the first aspect, the present invention covers compounds of general formula (Ib), supra, in which:

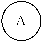

represents a:

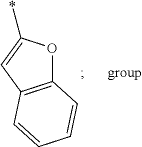 [SMILES: *c1cc2ccccc2o1]

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
n represents an integer of 1;
or a stereoisomer, a tautomer, an N-oxide, a hydrate, a solvate, or a salt thereof, or a mixture of same.

In accordance with a sixth embodiment of the second variant of the first aspect, the present invention covers compounds of general formula (Ib), supra, in which:

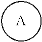

represents a:

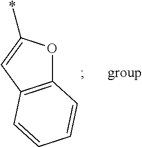 [SMILES: *c1cc2ccccc2o1]

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-alkoxy-C₁-C₆-alkyl-, C₁-C₆-hydroxyalkyl group;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
n represents an integer of 1;
or a stereoisomer, a tautomer, an N-oxide, a hydrate, a solvate, or a salt thereof, or a mixture of same.

In accordance with a seventh embodiment of the second variant of the first aspect, the present invention covers compounds of general formula (Ib), supra, in which:

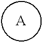

represents a:

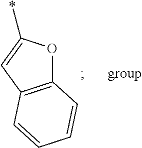 [SMILES: *c1cc2ccccc2o1]

wherein * indicates the point of attachment of said group with the rest of the molecule; and
R1 represents a linear C₁-C₆-alkyl-, or a C₃-C₁₀-cycloalkyl group; which is optionally substituted, one or more times, independently from each other, with a substituent selected from:
4- to 10-membered heterocycloalkyl- optionally substituted one or more times, independently from each other, with an R5 substituent; —NH₂, —N(H)C(═O)OR′, —S(═O)₂R′, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a C₁-C₆-alkyl-, C₁-C₆-alkoxy-C₁-C₆-alkyl-, aryl- optionally substituted one or more times, independently from each other, with a halogen atom, —OH, —CN, C₁-C₆-alkyl-, C₁-C₆-haloalkyl-, C₁-C₆-alkoxy group;
R6 represents a C₁-C₆-alkyl group;
R7 represents a C₁-C₆-alkoxy-C₁-C₆-alkyl group;
R′ and R″ represent, independently from each other, a substituent selected from:
a C₁-C₆-alkyl-, C₃-C₁₀-cycloalkyl-, C₁-C₆-haloalkyl group;
n represents an integer of 1;
or a stereoisomer, a tautomer, an N-oxide, a hydrate, a solvate, or a salt thereof, or a mixture of same.

In accordance with a variant of the seventh embodiment of the second variant of the first aspect, the present invention covers compounds of general formula (Ib), supra, in which:

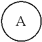

represents a:

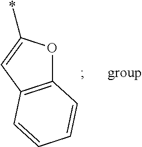 [SMILES: *c1cc2ccccc2o1]

wherein * indicates the point of attachment of said group with the rest of the molecule; and
R1 represents a linear C₁-C₆-alkyl-, or a C₃-C₁₀-cycloalkyl group; which is optionally substituted, one or more times, independently from each other, with a substituent selected from:
C₁-C₆-haloalkyl-, 4- to 10-membered heterocycloalkyl- optionally substituted one or more times, independently from each other, with an R5 substituent; —NH₂, —N(H)C(═O)OR′, —S(═O)₂R′, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a C₁-C₆-alkyl-, C₁-C₆-alkoxy-C₁-C₆-alkyl-, aryl- optionally substituted one or more times, independently from each other, with a halogen atom, —OH, —CN, C₁-C₆-alkyl-, C₁-C₆-haloalkyl-, C₁-C₆-alkoxy group;
R6 represents a C₁-C₆-alkyl group;
R7 represents a C₁-C₆-alkoxy-C₁-C₆-alkyl group;
R′ and R″ represent, independently from each other, a substituent selected from:
a C₁-C₆-alkyl-, C₃-C₁₀-cycloalkyl-, C₁-C₆-haloalkyl group;
n represents an integer of 1;
or a stereoisomer, a tautomer, an N-oxide, a hydrate, a solvate, or a salt thereof, or a mixture of same.

In a further embodiment of the above-mentioned second variant of the first aspect, the invention relates to compounds of formula (Ib), wherein:

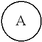

represents a:

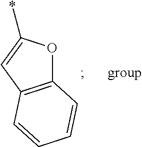 [SMILES: *c1cc2ccccc2o1]

wherein * indicates the point of attachment of said group with the rest of the molecule.

In a further embodiment of the above-mentioned second variant of the first aspect, the invention relates to compounds of formula (Ib), wherein:

R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent.

In a further embodiment of the above-mentioned second variant of the first aspect, the invention relates to compounds of formula (Ib), wherein:

R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group.

In a further embodiment of the above-mentioned second variant of the first aspect, the invention relates to compounds of formula (Ib), wherein:

R1 represents a 4- to 10-membered heterocycloalkyl group optionally substituted one or more times, independently from each other with an R5 substituent.

In a further embodiment of the above-mentioned second variant of the first aspect, the invention relates to compounds of formula (Ib), wherein:

R2 represents a hydrogen atom.

In a further embodiment of the above-mentioned second variant of the first aspect, the invention relates to compounds of formula (Ib), wherein:

R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group.

In a further embodiment of the above-mentioned second variant of the first aspect, the invention relates to compounds of formula (Ib), wherein:

R3 represents a N(R6)R7 group.

In a further embodiment of the above-mentioned second variant of the first aspect, the invention relates to compounds of formula (Ib), wherein:

R3 represents a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group.

In a further embodiment of the above-mentioned second variant of the first aspect, the invention relates to compounds of formula (Ib), wherein:

R4 represents a hydrogen atom, a halogen atom, a —CN, C₁-C₆-alkyl-, C₁-C₆-haloalkyl-, C₃-C₁₀-cycloalkyl-, 3- to 10-membered heterocycloalkyl- group.

In a further embodiment of the above-mentioned second variant of the first aspect, the invention relates to compounds of formula (Ib), wherein:

R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C1-C₆-haloalkyl-, C₁-C₆-alkoxy group.

In a further embodiment of the above-mentioned second variant of the first aspect, the invention relates to compounds of formula (Ib), wherein:

R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent.

In a further embodiment of the above-mentioned second variant of the first aspect, the invention relates to compounds of formula (Ib), wherein:

R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent.

In a further embodiment of the above-mentioned second variant of the first aspect, the invention relates to compounds of formula (Ib), wherein:

R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group.

In a further embodiment of the above-mentioned second variant of the first aspect, the invention relates to compounds of formula (Ib), wherein:

R′ and R″ represent, independently from each other, a substituent selected from:
a C₁-C₆-alkyl-, C₃-C₁₀-cycloalkyl-, C₁-C₆-haloalkyl group.

In a further embodiment of the above-mentioned second variant of the first aspect, the invention relates to compounds of formula (Ib), wherein:

R′″ and R″″ represent, independently from each other, a C₁-C₄-alkyl group.

In a further embodiment of the above-mentioned second variant of the first aspect, the invention relates to compounds of formula (Ib), wherein:

R′″″ represents a substituent selected from:
a C₁-C₄-alkyl group, phenyl.

In a further embodiment of the above-mentioned second variant of the first aspect, the invention relates to compounds of formula (Ib), wherein:

n represents an integer of 1, 2, 3, 4 or 5.

In a further embodiment of the above-mentioned second variant of the first aspect, the invention relates to compounds of formula (Ib), wherein:

n represents an integer of 1.

In a further embodiment of the above-mentioned second variant of the first aspect, the invention relates to compounds of formula (Ib), wherein:

n represents an integer of 2.

In a further embodiment of the above-mentioned second variant of the first aspect, the invention relates to compounds of formula (Ib), wherein:

n represents an integer of 3.

In a further embodiment of the above-mentioned second variant of the first aspect, the invention relates to compounds of formula (Ib), wherein:

n represents an integer of 4.

In a further embodiment of the above-mentioned second variant of the first aspect, the invention relates to compounds of formula (Ib), wherein:

n represents an integer of 5.

In a further embodiment of the above-mentioned second variant of the first aspect, the invention relates to compounds of formula (Ib), wherein:

R4 represents a hydrogen atom.

In a further embodiment of the above-mentioned second variant of the first aspect, the invention relates to compounds of formula (Ib), wherein:

R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent.

In a further embodiment of the above-mentioned second variant of the first aspect, the invention relates to compounds of formula (Ib), wherein:

R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group.

In a further embodiment of the above-mentioned second variant of the first aspect, the invention relates to compounds of formula (Ib), wherein:

R5 represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, aryl- optionally substituted one or more times, independently from each other, with a halogen atom, —OH, —CN, C₁-C₆-alkyl-, C1-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group.

In a further embodiment of the above-mentioned second variant of the first aspect, the invention relates to compounds of formula (Ib), wherein:

R6 represents a substituent selected from:
a hydrogen atom, a C₁-C₆-alkyl-, C₃-C₁₀-cycloalkyl-, C₁-C₆-alkoxy-C₁-C₆-alkyl-, C₁-C₆-hydroxyalkyl group.

In a further embodiment of the above-mentioned second variant of the first aspect, the invention relates to compounds of formula (Ib), wherein:

R1 represents a linear C₁-C₆-alkyl-, or a C₃-C₁₀-cycloalkyl group; which is optionally substituted, one or more times, independently from each other, with a substituent selected from:
4- to 10-membered heterocycloalkyl- optionally substituted one or more times, independently from each other, with an R5 substituent; —NH₂, —N(H)C(═O)OR′, —S(═O)₂R′, —S(═O)(═N(CN))R″ group;
or a 4- to 10-membered heterocycloalkyl group optionally substituted one or more times, independently from each other with an R5 substituent.

In a further embodiment of the above-mentioned second variant of the first aspect, the invention relates to compounds of formula (Ib), wherein:

R1 represents a linear C₁-C₆-alkyl-, or a C₃-C₁₀-cycloalkyl group; which is optionally substituted, one or more times, independently from each other, with a substituent selected from:
4- to 10-membered heterocycloalkyl- optionally substituted one or more times, independently from each other, with an R5 substituent; —NH₂, —N(H)C(═O)OR′, —S(═O)₂R′, —S(═O)(═N(CN))R″ group.

In a further embodiment of the above-mentioned second variant of the first aspect, the invention relates to compounds of formula (Ib), wherein:

R1 represents a linear C₁-C₆-alkyl-, or a C₃-C₁₀-cycloalkyl group; which is optionally substituted, one or more times, independently from each other, with a substituent selected from:
C₁-C₆-haloalkyl-, 4- to 10-membered heterocycloalkyl- optionally substituted one or more times, independently from each other, with an R5 substituent; —NH₂, —N(H)C(═O)OR′, —S(═O)₂R′, —S(═O)(═N(CN))R″ group;
or a 4- to 10-membered heterocycloalkyl group optionally substituted one or more times, independently from each other with an R5 substituent.

In a further embodiment of the above-mentioned second variant of the first aspect, the invention relates to compounds of formula (Ib), wherein:

R1 represents a linear C₁-C₆-alkyl-, or a C₃-C₁₀-cycloalkyl group; which is optionally substituted, one or more times, independently from each other, with a substituent selected from:
C₁-C₆-haloalkyl-, 4- to 10-membered heterocycloalkyl- optionally substituted one or more times, independently from each other, with an R5 substituent; —NH₂, —N(H)C(═O)OR′, —S(═O)₂R′, —S(═O)(═N(CN))R″ group.

In a further embodiment of the above-mentioned second variant of the first aspect, the invention relates to compounds of formula (Ib), wherein:

R5 represents a substituent selected from:
a C₁-C₆-alkyl-, C₁-C₆-alkoxy-C₁-C₆-alkyl-, aryl- optionally substituted one or more times, independently from each other, with a halogen atom, —OH, —CN, C₁-C₆-alkyl-, C₁-C₆-haloalkyl-, C₁-C₆-alkoxy group.

In a further embodiment of the above-mentioned second variant of the first aspect, the invention relates to compounds of formula (Ib), wherein:

R6 represents a C₁-C₆-alkyl group.

In a further embodiment of the above-mentioned second variant of the first aspect, the invention relates to compounds of formula (Ib), wherein:

R7 represents a C₁-C₆-alkoxy-C₁-C₆-alkyl group.

In a further embodiment of the above-mentioned second variant of the first aspect, the invention relates to compounds of formula (Ib), wherein:

R′ and R″ represent, independently from each other, a substituent selected from:
a C₁-C₆-alkyl-, C₃-C₁₀-cycloalkyl-, C₁-C₆-haloalkyl group.

In accordance with a further aspect, the present invention covers intermediate compounds which are useful in the preparation of compounds of the present invention of general formula (Ib), particularly in the method described herein. In particular, the present invention covers compounds of general formula (Eb):

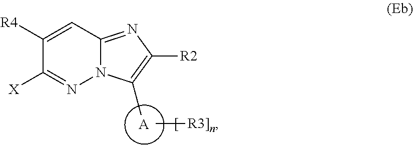

in which A, R2, R3, R4 and n are as defined for the compound of general formula (Ib) supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Ib), particularly in the method described herein. In particular, the present invention covers compounds of general formula (Hb):

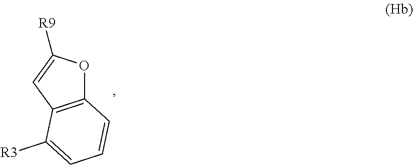

in which R3 is as defined for the compound of general formula (Ib) supra, and in which R9 represents a boronic acid —B(OH)₂, or a boronic acid ester.

In accordance with a further aspect, the present invention covers intermediate compounds which are useful in the preparation of compounds of the present invention of general formula (Ib), particularly in the method described herein. In particular, the present invention covers compounds of general formula (Jb):

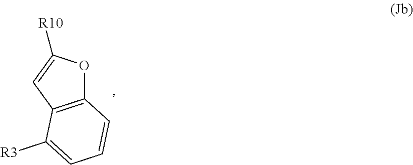

in which R3 is as defined for the compound of general formula (Ib) supra, and in which R10 represents a stannyl group, such as a tri-n-butylstannyl group for example.

In accordance with yet another aspect, the present invention covers the use of the intermediate compounds of general formula (Eb):

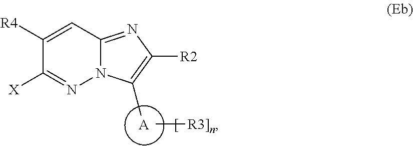

in which A, R2, R3, R4 and n are as defined for the compound of general formula (Ib) supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group for example, for the preparation of a compound of general formula (Ib) as defined supra.

In accordance with yet another aspect, the present invention covers the use of the intermediate compounds of general formula (Eb′):

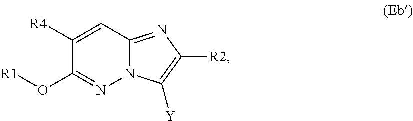

in which R1, R2 and R4 are as defined for the compound of general formula (Ib) supra, and in which Y represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Ib) as defined supra.

In accordance with yet another aspect, the present invention covers the use of the intermediate compounds of general formula (Hb):

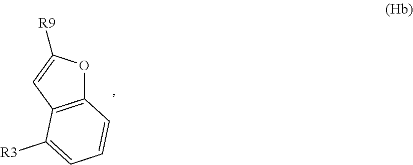

in which R3 is as defined for the compound of general formula (Ib) supra, and in which R9 represents a boronic acid —B(OH)₂, or a boronic acid ester, for the preparation of a compound of general formula (Ib) as defined supra.

In accordance with yet another aspect, the present invention covers the use of the intermediate compounds of general formula (Jb):

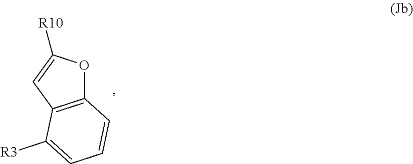

in which R3 is as defined for the compound of general formula (Ib) supra, and in which R10 represents a stannyl group, such as a tri-n-butylstannyl group, for example for the preparation of a compound of general formula (Ib) as defined supra.

In accordance with a second embodiment of the third variant of the first aspect, the present invention covers compounds of general formula (Ic), supra, in which:

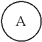

represents a group selected from:

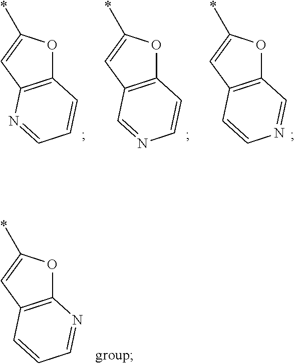

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom, a halogen atom, a —CN, C₁-C₆-alkyl-, C₁-C₆-haloalkyl-, C₃-C₁₀-cycloalkyl-, 3- to 10-membered heterocycloalkyl group;
R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1;
or a stereoisomer, a tautomer, an N-oxide, a hydrate, a solvate, or a salt thereof, or a mixture of same.

In accordance with a third embodiment of the third variant of the first aspect, the present invention covers compounds of general formula (Ic), supra, in which:

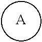

represents a group selected from:

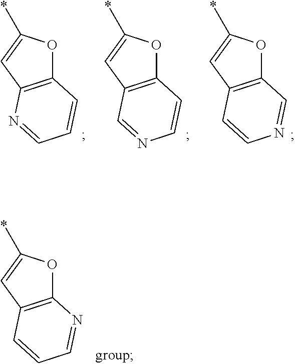

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C1-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1;
or a stereoisomer, a tautomer, an N-oxide, a hydrate, a solvate, or a salt thereof, or a mixture of same.

In accordance with a fourth embodiment of the third variant of the first aspect, the present invention covers compounds of general formula (Ic), supra, in which:

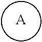

represents a group selected from:

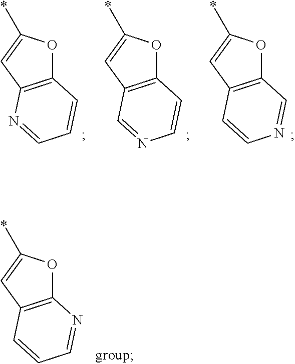

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent; R2 represents a hydrogen atom; R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C1-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1;
or a stereoisomer, a tautomer, an N-oxide, a hydrate, a solvate, or a salt thereof, or a mixture of same.

In accordance with a fifth embodiment of the third variant of the first aspect, the present invention covers compounds of general formula (Ic), supra, in which:

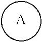

represents a group selected from:

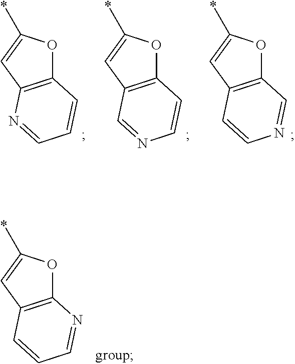

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1;
or a stereoisomer, a tautomer, an N-oxide, a hydrate, a solvate, or a salt thereof, or a mixture of same.

In accordance with a sixth embodiment of the third variant of the first aspect, the present invention covers compounds of general formula (Ic), supra, in which:

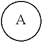

represents a group selected from:

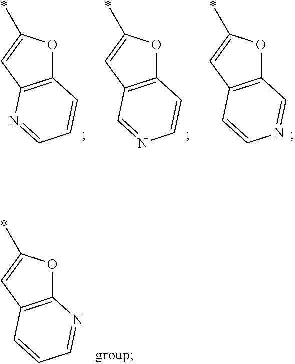

wherein * indicates the point of attachment of said group with the rest of the molecule; and
R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 represents a substituent selected from:
a hydrogen atom, a C₁-C₆-alkyl-, C₃-C₁₀-cycloalkyl-, C₁-C₆-alkoxy-C₁-C₆-alkyl-, C₁-C₆-hydroxyalkyl group;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ represent, independently from each other, a substituent selected from:
a C₁-C₆-alkyl-, C₃-C₁₀-cycloalkyl-, C₁-C₆-haloalkyl group;
R′″ and R″″ represent, independently from each other, a C₁-C₄-alkyl group;
R′″″ represents a substituent selected from:
a C₁-C₄-alkyl group, phenyl;
n represents an integer of 1;
or a stereoisomer, a tautomer, an N-oxide, a hydrate, a solvate, or a salt thereof, or a mixture of same.

In accordance with a seventh embodiment of the third variant of the first aspect, the present invention covers compounds of general formula (Ic), supra, in which:

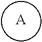

represents a

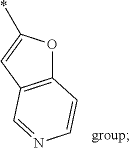 [SMILES: *c1cc2cnccc2o1]

wherein * indicates the point of attachment of said group with the rest of the molecule; and
R1 represents a linear C₁-C₆-alkyl-, or a C₃-C₁₀-cycloalkyl group; which is optionally substituted, one or more times, independently from each other, with a substituent selected from:
4- to 10-membered heterocycloalkyl- optionally substituted one or more times, independently from each other, with an R5 substituent; —NH₂, —S(═O)₂R′ group;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a C₁-C₆-alkyl-, C₁-C₆-hydroxyalkyl-, C₁-C₆-alkoxy-C₁-C₆-alkyl-, —C(═O)R′, —C(═O)OR′, —N(R′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group;
R6 represents a C₁-C₆-alkyl group;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R′ and R″ represent, independently from each other, a C₁-C₆-alkyl group;
R′″ and R″″ represent, independently from each other, a C₁-C₄-alkyl group;
R′″″ represents a C₁-C₄-alkyl group;
n represents an integer of 1;
or a stereoisomer, a tautomer, an N-oxide, a hydrate, a solvate, or a salt thereof, or a mixture of same.

In accordance with a variant of the seventh embodiment of the third variant of the first aspect, the present invention covers compounds of general formula (Ic), supra, in which:

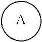

represents a

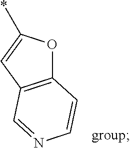 [SMILES: *c1cc2cnccc2o1]

wherein * indicates the point of attachment of said group with the rest of the molecule; and
R1 represents a linear C₁-C₆-alkyl-, or a C₃-C₁₀-cycloalkyl group; which is optionally substituted, one or more times, independently from each other, with a substituent selected from:
C₁-C₆-alkyl-, C₁-C₆-haloalkyl-, 4- to 10-membered heterocycloalkyl- optionally substituted one or more times, independently from each other, with an R5 substituent; —NH₂, —S(═O)₂R′, —S(═O)(═N(CN))R″ group;
R2 represents a hydrogen atom;
R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 represents a hydrogen atom;
R5 represents a substituent selected from:
a C₁-C₆-alkyl-, C₁-C₆-hydroxyalkyl-, C₁-C₆-alkoxy-C₁-C₆-alkyl-, —C(═O)R′, —C(═O)OR′, —N(R′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group;
R6 represents a C₁-C₆-alkyl group;
R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R′ and R″ represent, independently from each other, a C₁-C₆-alkyl group;
R′″ and R″″ represent, independently from each other, a C₁-C₄-alkyl group;
R′″″ represents a C₁-C₄-alkyl group;
n represents an integer of 1;
or a stereoisomer, a tautomer, an N-oxide, a hydrate, a solvate, or a salt thereof, or a mixture of same.

In a further embodiment of the above-mentioned third variant of the first aspect, the invention relates to compounds of formula (Ic), wherein:

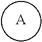

represents a group selected from:

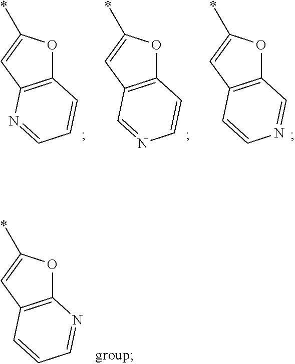

wherein * indicates the point of attachment of said group with the rest of the molecule.

In a further embodiment of the above-mentioned third variant of the first aspect, the invention relates to compounds of formula (Ic), wherein:

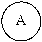

represents a

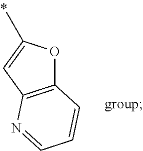

wherein * indicates the point of attachment of said group with the rest of the molecule.

In a further embodiment of the above-mentioned third variant of the first aspect, the invention relates to compounds of formula (Ic), wherein:

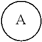

represents a

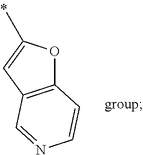

wherein * indicates the point of attachment of said group with the rest of the molecule.

In a further embodiment of the above-mentioned third variant of the first aspect, the invention relates to compounds of formula (Ic), wherein:

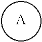

represents a

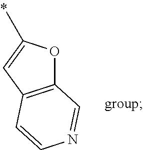

wherein * indicates the point of attachment of said group with the rest of the molecule.

In a further embodiment of the above-mentioned third variant of the first aspect, the invention relates to compounds of formula (Ic), wherein:

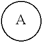

represents a

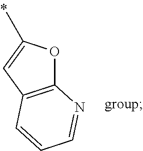

wherein * indicates the point of attachment of said group with the rest of the molecule.

In a further embodiment of the above-mentioned third variant of the first aspect, the invention relates to compounds of formula (Ic), wherein:

R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent.

In a further embodiment of the above-mentioned third variant of the first aspect, the invention relates to compounds of formula (Ic), wherein:

R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group.

In a further embodiment of the above-mentioned third variant of the first aspect, the invention relates to compounds of formula (Ic), wherein:

R1 represents a 4- to 10-membered heterocycloalkyl group optionally substituted one or more times, independently from each other with an R5 substituent.

In a further embodiment of the above-mentioned third variant of the first aspect, the invention relates to compounds of formula (Ic), wherein:

R2 represents a hydrogen atom.

In a further embodiment of the above-mentioned third variant of the first aspect, the invention relates to compounds of formula (Ic), wherein:

R3 represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group.

In a further embodiment of the above-mentioned third variant of the first aspect, the invention relates to compounds of formula (Ic), wherein:

R3 represents a N(R6)R7 group.

In a further embodiment of the above-mentioned third variant of the first aspect, the invention relates to compounds of formula (Ic), wherein:

R3 represents a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group.

In a further embodiment of the above-mentioned third variant of the first aspect, the invention relates to compounds of formula (Ic), wherein:

R4 represents a hydrogen atom, a halogen atom, a —CN, C₁-C₆-alkyl-, C₁-C₆-haloalkyl-, C₃-C₁₀-cycloalkyl-, 3- to 10-membered heterocycloalkyl group.

In a further embodiment of the above-mentioned third variant of the first aspect, the invention relates to compounds of formula (Ic), wherein:

R5 represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C1-C₆-haloalkyl-, C₁-C₆-alkoxy group.

In a further embodiment of the above-mentioned third variant of the first aspect, the invention relates to compounds of formula (Ic), wherein:

R6 represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent.

In a further embodiment of the above-mentioned third variant of the first aspect, the invention relates to compounds of formula (Ic), wherein:

R7 represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent.

In a further embodiment of the above-mentioned third variant of the first aspect, the invention relates to compounds of formula (Ic), wherein:

R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group.

In a further embodiment of the above-mentioned third variant of the first aspect, the invention relates to compounds of formula (Ic), wherein:

R′ and R″ represent, independently from each other, a substituent selected from:
a C₁-C₆-alkyl-, C₃-C₁₀-cycloalkyl-, C₁-C₆-haloalkyl group.

In a further embodiment of the above-mentioned third variant of the first aspect, the invention relates to compounds of formula (Ic), wherein:

R′″ and R″″ represent, independently from each other, a C₁-C₄-alkyl group.

In a further embodiment of the above-mentioned third variant of the first aspect, the invention relates to compounds of formula (Ic), wherein:

R′″″ represents a substituent selected from:
a C₁-C₄-alkyl group, phenyl.

In a further embodiment of the above-mentioned third variant of the first aspect, the invention relates to compounds of formula (Ic), wherein:

n represents an integer of 1, 2, 3 or 4.

In a further embodiment of the above-mentioned third variant of the first aspect, the invention relates to compounds of formula (Ic), wherein:

n represents an integer of 1.

In a further embodiment of the above-mentioned third variant of the first aspect, the invention relates to compounds of formula (Ic), wherein:

n represents an integer of 2.

In a further embodiment of the above-mentioned third variant of the first aspect, the invention relates to compounds of formula (Ic), wherein:

n represents an integer of 3.

In a further embodiment of the above-mentioned third variant of the first aspect, the invention relates to compounds of formula (Ic), wherein:

n represents an integer of 4.

In a further embodiment of the above-mentioned third variant of the first aspect, the invention relates to compounds of formula (Ic), wherein:

R4 represents a hydrogen atom.

In a further embodiment of the above-mentioned third variant of the first aspect, the invention relates to compounds of formula (Ic), wherein:

R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent.

In a further embodiment of the above-mentioned third variant of the first aspect, the invention relates to compounds of formula (Ic), wherein:

R1 represents a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group.

In a further embodiment of the above-mentioned third variant of the first aspect, the invention relates to compounds of formula (Ic), wherein:

R5 represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group.

In a further embodiment of the above-mentioned third variant of the first aspect, the invention relates to compounds of formula (Ic), wherein:

R6 represents a substituent selected from:
a hydrogen atom, a C₁-C₆-alkyl-, C₃-C₁₀-cycloalkyl-, C₁-C₆-alkoxy-C₁-C₆-alkyl-, C₁-C₆-hydroxyalkyl group.

In a further embodiment of the above-mentioned third variant of the first aspect, the invention relates to compounds of formula (Ic), wherein:

R1 represents a linear C₁-C₆-alkyl-, or a C₃-C₁₀-cycloalkyl group; which is optionally substituted, one or more times, independently from each other, with a substituent selected from:
4- to 10-membered heterocycloalkyl- optionally substituted one or more times, independently from each other, with an R5 substituent; —NH₂, —S(═O)₂R′ group.

In a further embodiment of the above-mentioned third variant of the first aspect, the invention relates to compounds of formula (Ic), wherein:

R1 represents a linear C₁-C₆-alkyl-, or a C₃-C₁₀-cycloalkyl group; which is optionally substituted, one or more times, independently from each other, with a substituent selected from:
C₁-C₆-alkyl-, C₁-C₆-haloalkyl-, 4- to 10-membered heterocycloalkyl- optionally substituted one or more times, independently from each other, with an R5 substituent; —NH₂, —S(═O)₂R′, —S(═O)(═N(CN))R″ group.

In a further embodiment of the above-mentioned third variant of the first aspect, the invention relates to compounds of formula (Ic), wherein:

R5 represents a substituent selected from:
a C₁-C₆-alkyl-, C₁-C₆-hydroxyalkyl-, C₁-C₆-alkoxy-C₁-C₆-alkyl-, —C(═O)R′, —C(═O)OR′, —N(R′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group.

In a further embodiment of the above-mentioned third variant of the first aspect, the invention relates to compounds of formula (Ic), wherein:

R6 represents a C₁-C₆-alkyl group.

In a further embodiment of the above-mentioned third variant of the first aspect, the invention relates to compounds of formula (Ic), wherein:

R′ and R″ represent, independently from each other, a C₁-C₆-alkyl group.

In a further embodiment of the above-mentioned third variant of the first aspect, the invention relates to compounds of formula (Ic), wherein:

R′″″ represents a C₁-C₄-alkyl group.

In a further embodiment of the above-mentioned third variant of the first aspect, the invention relates to compounds of formula (Ic), according to any of the above-mentioned embodiments, in the form of or a stereoisomer, a tautomer, an N-oxide, a hydrate, a solvate, or a salt thereof, or a mixture of same.

In accordance with a further aspect, the present invention covers intermediate compounds which are useful in the preparation of compounds of the present invention of general formula (Ic), particularly in the method described herein. In particular, the present invention covers compounds of general formula (Ec):

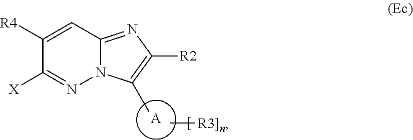

in which A, R2, R3, R4 and n are as defined for the compound of general formula (Ic) supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Ic), particularly in the method described herein. In particular, the present invention covers compounds of general formula (Hc):

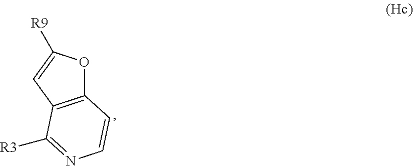

in which R3 is as defined for the compound of general formula (Ic) supra, and in which R9 represents a boronic acid —B(OH)₂, or a boronic acid ester.

In accordance with a further aspect, the present invention covers intermediate compounds which are useful in the preparation of compounds of the present invention of general formula (Ic), particularly in the method described herein. In particular, the present invention covers compounds of general formula (Jc):

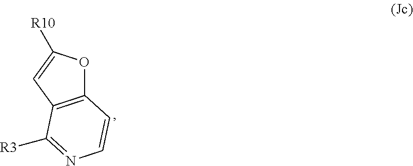

in which R3 is as defined for the compound of general formula (Ic) supra, and in which R10 represents a stannyl group, such as a tri-n-butylstannyl group for example.

In accordance with yet another aspect, the present invention covers the use of the intermediate compounds of general formula (Ec):

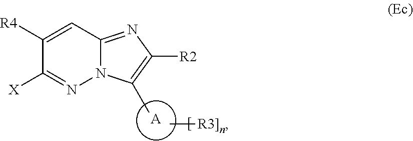

in which A, R2, R3, R4 and n are as defined for the compound of general formula (Ic) supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group for example, for the preparation of a compound of general formula (Ic) as defined supra.

In accordance with yet another aspect, the present invention covers the use of the intermediate compounds of general formula (Ec′):

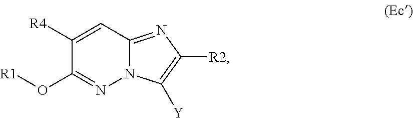

in which R1, R2 and R4 are as defined for the compound of general formula (Ic) supra, and in which Y represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Ic) as defined supra.

In accordance with yet another aspect, the present invention covers the use of the intermediate compounds of general formula (Hc):

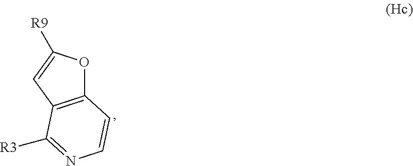

in which R3 is as defined for the compound of general formula (Ic) supra, and in which R9 represents a boronic acid —B(OH)₂, or a boronic acid ester, for the preparation of a compound of general formula (Ic) as defined supra.

In accordance with yet another aspect, the present invention covers the use of the intermediate compounds of general formula (Jc):

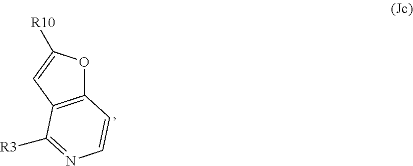

in which R3 is as defined for the compound of general formula (Ic) supra, and in which R10 represents a stannyl group, such as a tri-n-butylstannyl group for example. In accordance with a second embodiment of the fourth variant of the first aspect, the resent invention covers compounds of general formula (Id), supra, in which:

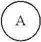

represents a group selected from:

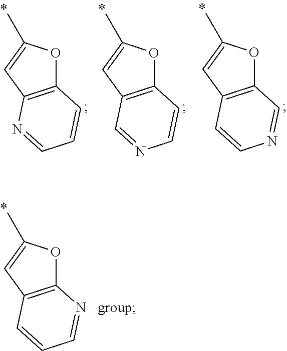

wherein * indicates the point of attachment of said group with the rest of the molecule; and

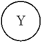

represents a:

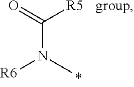

or a

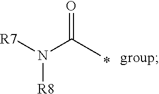

wherein * indicates the point of attachment of said group to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R2 represents a hydrogen atom;
R3 represents a substituent selected from:
a halogen atom, a —CN, C₁-C₆-alkyl-, C₁-C₆-haloalkyl-, C₂-C₆-alkenyl-, C₂-C₆-alkynyl-, —C(═O)R′, —C(═O)NH₂, —C(═O)N(H)R′, —C(═O)N(R′)R″, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₃-C₆-cycloalkyl-C₁-C₆-alkoxy-, C₁-C₆-haloalkoxy-, —OC(═O)R′, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R4 represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8, together with a carbon atom of R1, represent a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0, 1, 2, 3 or 4;
   or a stereoisomer, a tautomer, an N-oxide, a hydrate, a solvate, or a salt thereof, or a mixture of same.

In accordance with a third embodiment of the fourth variant of the first aspect, the present invention covers compounds of general formula (Id), supra, in which:

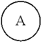

represents a group selected from:

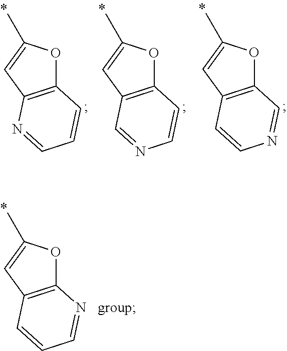

wherein * indicates the point of attachment of said group with the rest of the molecule; and

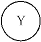

represents a:

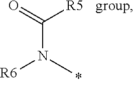

or a

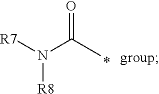

wherein * indicates the point of attachment of said group to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R2 represents a hydrogen atom;
R3 represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group;
R4 represents a substituent selected from:
a hydrogen atom, a halogen atom, a —CN, C₁-C₆-alkyl-, C₁-C₆-haloalkyl-,
C₃-C₁₀-cycloalkyl-, aryl-; heteroaryl- group;
R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8, together with a carbon atom of R1, represent a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0 or 1;
   or a stereoisomer, a tautomer, an N-oxide, a hydrate, a solvate, or a salt thereof, or a mixture of same.

In accordance with a fourth embodiment of the fourth variant of the first aspect, the present invention covers compounds of general formula (Id), supra, in which:

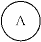

represents a group selected from:

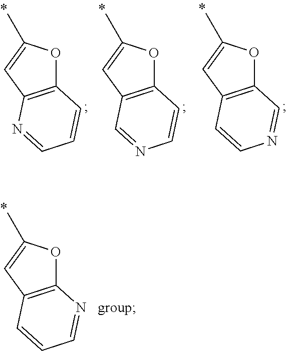

wherein * indicates the point of attachment of said group with the rest of the molecule; and

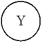

represents a:

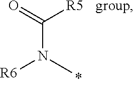

or a

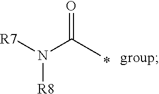

wherein * indicates the point of attachment of said group to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 represents a hydrogen atom;
R3 represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C1-C₆-alkoxy-, C₁-C₆-haloalkoxy- group;
R4 represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8, together with a carbon atom of R1, represent a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0 or 1;
   or a stereoisomer, a tautomer, an N-oxide, a hydrate, a solvate, or a salt thereof, or a mixture of same.

In accordance with a fifth embodiment of the fourth variant of the first aspect, the present invention covers compounds of general formula (Id), supra, in which:

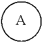

represents a group selected from:

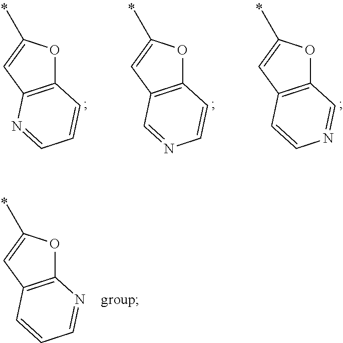

wherein * indicates the point of attachment of said group with the rest of the molecule; and

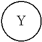

represents a:

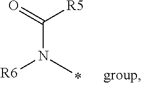

or a

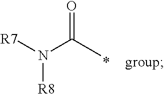

wherein * indicates the point of attachment of said group to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 represents a hydrogen atom;
R3 represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group;
R4 represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 represents:
either:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
R7 or R8, together with a carbon atom of R1, represent a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0 or 1;
   or a stereoisomer, a tautomer, an N-oxide, a hydrate, a solvate, or a salt thereof, or a mixture of same.

In accordance with a sixth embodiment of the fourth variant of the first aspect, the present invention covers compounds of general formula (Id), supra, in which:

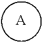

represents a group selected from:

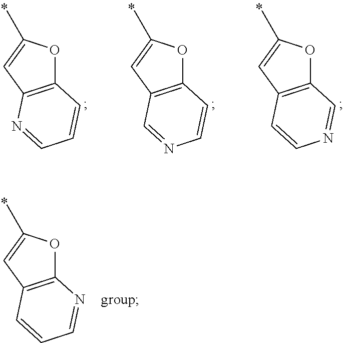

wherein * indicates the point of attachment of said group with the rest of the molecule; and

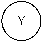

represents a:

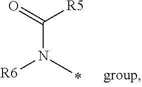

or a

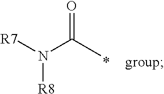

wherein * indicates the point of attachment of said group to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 represents a hydrogen atom;
R3 represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group;
R4 represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 represents:
either:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₁₀-cycloalkyl-, C₃-C₁₀-cycloalkyl-C₁-C₆-alkyl-, aryl- optionally substituted one or more times, independently from each other, with an R substituent; heteroaryl-optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl-, C₃-C₁₀-cycloalkyl-, C₁-C₆-haloalkyl group;
   or:
together, a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R7 or R8, together with a carbon atom of R1, represent a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0 or 1;
   or a stereoisomer, a tautomer, an N-oxide, a hydrate, a solvate, or a salt thereof, or a mixture of same.

In accordance with a seventh embodiment of the fourth variant of the first aspect, the present invention covers compounds of general formula (Id), supra, in which:

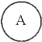

represents a group selected from:

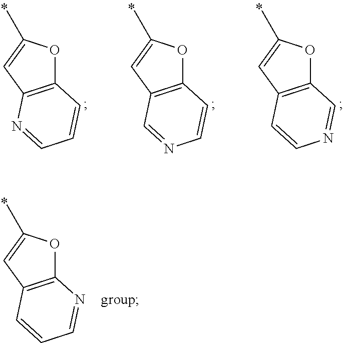

wherein * indicates the point of attachment of said group with the rest of the molecule; and

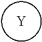

represents a:

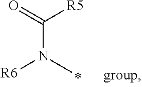

or a

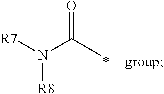

wherein * indicates the point of attachment of said group to R1; and
R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 represents a hydrogen atom;
R3 represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C1-C₆-alkoxy-, C₁-C₆-haloalkoxy- group;
R4 represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 represents:
either:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
   R6 represents:
   either:
a substituent selected from hydrogen or a C₁-C₆-alkyl-, C₁-C₆-haloalkyl-, C₃-C₁₀-cycloalkyl-, C₃-C₁₀-cycloalkyl-C₁-C₆-alkyl-, aryl- optionally substituted one or more times, independently from each other, with an R substituent; heteroaryl-optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
   R7 and R8 represent:
   either:
independently from each other, a substituent selected from:
a hydrogen atom, a C₁-C₆-alkyl- group;
   or:
together, a 4-, 5-, 6- or 7-membered cyclic amine group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 6- or 7-membered cyclic amine group optionally containing one further heteroatom selected from the group consisting of O, N and S;
   or:
R7 or R8, together with a carbon atom of R1, represent a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
   R represents a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0 or 1;
   or a stereoisomer, a tautomer, an N-oxide, a hydrate, a solvate, or a salt thereof, or a mixture of same.

In accordance with a eighth embodiment of the fourth variant of the first aspect, the present invention covers compounds of general formula (Id), supra, in which:

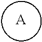

represents a:

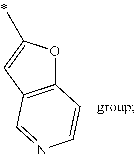

wherein * indicates the point of attachment of said group with the rest of the molecule; and

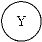

represents a:

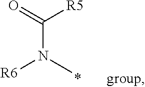

or a

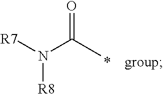

wherein * indicates the point of attachment of said group to R1; and
R1 represents a linear C₁-C₆-alkyl- group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 represents a hydrogen atom;
R3 represents a substituent selected from:
—NHR′, C₁-C₆-alkoxy-, C₃-C₆-cycloalkyl-C₁-C₆-alkoxy- group;
R4 represents a hydrogen atom;
R5 represents:
either:
a substituent selected from a C₁-C₆-alkyl-, C₁-C₆-haloalkyl-, C₃-C₁₀-cycloalkyl
   or:
together with a carbon atom of R1, represents a 6-membered cyclic amide group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 6-membered cyclic amide group optionally containing one further nitrogen atom;
   R6 represents:
   either a hydrogen atom,
   or:
together with a carbon atom of R1, represents a 5- or 6-membered cyclic amine group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6-membered cyclic amine group optionally containing one further oxygen atom;
   R7 and R8 represent independently from each other, a substituent selected from:
a hydrogen atom, a C₁-C₆-alkyl- group;
   R represents a C₁-C₆-alkyl- group;
   R′ and R″ represent, independently from each other, a substituent selected from:
   a C₁-C₆-alkyl-, C₃-C₁₀-cycloalkyl-, C₁-C₆-haloalkyl group;
   n represents an integer of 0 or 1;
   or a stereoisomer, a tautomer, an N-oxide, a hydrate, a solvate, or a salt thereof, or a mixture of same.

In a further embodiment of the above-mentioned fourth variant of the first aspect, the invention relates to compounds of formula (Id), wherein:

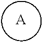

represents a group selected from:

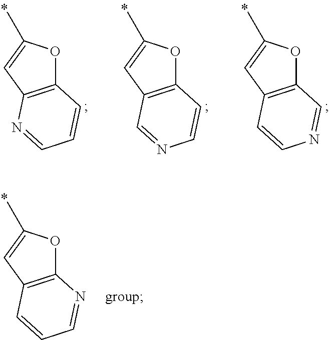

wherein * indicates the point of attachment of said group with the rest of the molecule.

In a further embodiment of the above-mentioned fourth variant of the first aspect, the invention relates to compounds of formula (Id), wherein:

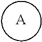

represents a group selected from:

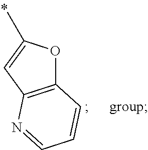

wherein * indicates the point of attachment of said group with the rest of the molecule.

In a further embodiment of the above-mentioned fourth variant of the first aspect, the invention relates to compounds of formula (Id), wherein:

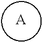

represents a group selected from:

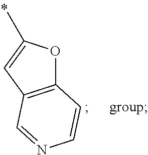

wherein * indicates the point of attachment of said group with the rest of the molecule.

In a further embodiment of the above-mentioned fourth variant of the first aspect, the invention relates to compounds of formula (Id), wherein:

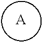

represents a group selected from:

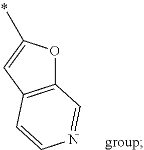

wherein * indicates the point of attachment of said group with the rest of the molecule.

In a further embodiment of the above-mentioned fourth variant of the first aspect, the invention relates to compounds of formula (Id), wherein:

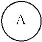

represents a group selected from:

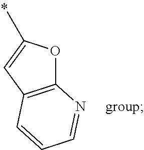

wherein * indicates the point of attachment of said group with the rest of the molecule.

In a further embodiment of the above-mentioned fourth variant of the first aspect, the invention relates to compounds of formula (Id), wherein:

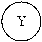

represents a:

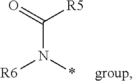

or a

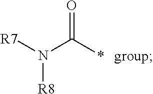

wherein * indicates the point of attachment of said group to R1.

In a further embodiment of the above-mentioned fourth variant of the first aspect, the invention relates to compounds of formula (Id), wherein:

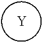

represents a:

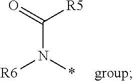

wherein * indicates the point of attachment of said group to R1.

In a further embodiment of the above-mentioned fourth variant of the first aspect, the invention relates to compounds of formula (Id), wherein:

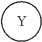

represents a:

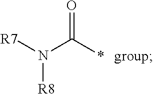

wherein * indicates the point of attachment of said group to R1.

In a further embodiment of the above-mentioned fourth variant of the first aspect, the invention relates to compounds of formula (Id), wherein:

R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group.

In a further embodiment of the above-mentioned fourth variant of the first aspect, the invention relates to compounds of formula (Id), wherein:

R1 represents a linear C₁-C₆-alkyl-, a branched C₃-C₆-alkyl-, or a C₃-C₆-cycloalkyl group.

In a further embodiment of the above-mentioned fourth variant of the first aspect, the invention relates to compounds of formula (Id), wherein:

R2 represents a hydrogen atom.

In a further embodiment of the above-mentioned fourth variant of the first aspect, the invention relates to compounds of formula (Id), wherein:

R3 represents a substituent selected from:
a halogen atom, a —CN, C₁-C₆-alkyl-, C₁-C₆-haloalkyl-, C₂-C₆-alkenyl-, C₂-C₆-alkynyl-, —C(═O)R′, —C(═O)NH₂, —C(═O)N(H)R′, —C(═O)N(R′)R″, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₃-C₆-cycloalkyl-C₁-C₆-alkoxy-, C₁-C₆-haloalkoxy-, —OC(═O)R′, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group.

In a further embodiment of the above-mentioned fourth variant of the first aspect, the invention relates to compounds of formula (Id), wherein:

R4 represents a substituent selected from:
a hydrogen atom, a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group.

In a further embodiment of the above-mentioned fourth variant of the first aspect, the invention relates to compounds of formula (Id), wherein:

R5 represents:
either:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
   or:
together with a carbon atom of R1, represents a 4-, 5-, 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R5 represents:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent.

In a further embodiment of the above-mentioned fourth variant of the first aspect, the invention relates to compounds of formula (Id), wherein:

R5, together with a carbon atom of R1, represents:
a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R6 represents:
either:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group.
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R6 represents:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl- optionally substituted one or more times, independently from each other, with an R substituent; heteroaryl- optionally substituted one or more times, independently from each other, with an R substituent; group.

In a further embodiment of the above-mentioned fourth variant of the first aspect, the invention relates to compounds of formula (Id), wherein:

R6, together with a carbon atom of R1, represents:
a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S.

In a further embodiment of the above-mentioned fourth variant of the first aspect, the invention relates to compounds of formula (Id), wherein:

R5 and R6 together represent:
a 4-, 5-, or 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R7 and R8 represent:
independently from each other, a substituent selected from:
a hydrogen atom, a C₁-C₆-alkyl-, C₃-C₁₀-cycloalkyl-, C₁-C₆-haloalkyl group.

In a further embodiment of the above-mentioned fourth variant of the first aspect, the invention relates to compounds of formula (Id), wherein:

R7 and R8 together represent:
a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S.

In a further embodiment of the above-mentioned fourth variant of the first aspect, the invention relates to compounds of formula (Id), wherein:

R7 or R8, together with a carbon atom of R1, represents:
a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group.

In a further embodiment of the above-mentioned fourth variant of the first aspect, the invention relates to compounds of formula (Id), wherein:

R′ and R″ represent, independently from each other, a substituent selected from:
a C₁-C₆-alkyl-, C₃-C₁₀-cycloalkyl-, C₁-C₆-haloalkyl group.

In a further embodiment of the above-mentioned fourth variant of the first aspect, the invention relates to compounds of formula (Id), wherein:

n represents an integer of 0, 1, 2, 3 or 4.

In a further embodiment of the above-mentioned fourth variant of the first aspect, the invention relates to compounds of formula (Id), wherein:

n represents an integer of 0.

In a further embodiment of the above-mentioned fourth variant of the first aspect, the invention relates to compounds of formula (Id), wherein:

n represents an integer of 1.

In a further embodiment of the above-mentioned fourth variant of the first aspect, the invention relates to compounds of formula (Id), wherein:

n represents an integer of 2.

In a further embodiment of the above-mentioned fourth variant of the first aspect, the invention relates to compounds of formula (Id), wherein:

n represents an integer of 3.

In a further embodiment of the above-mentioned fourth variant of the first aspect, the invention relates to compounds of formula (Id), wherein:

n represents an integer of 4.

In a further embodiment of the above-mentioned fourth variant of the first aspect, the invention relates to compounds of formula (Id), wherein:

R4 represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group.

In a further embodiment of the above-mentioned fourth variant of the first aspect, the invention relates to compounds of formula (Id), wherein:

R3 represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group.

In a further embodiment of the above-mentioned fourth variant of the first aspect, the invention relates to compounds of formula (Id), wherein:

n represents an integer of 0 or 1.

In a further embodiment of the above-mentioned fourth variant of the first aspect, the invention relates to compounds of formula (Id), wherein:

R1 represents a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group.

In a further embodiment of the above-mentioned fourth variant of the first aspect, the invention relates to compounds of formula (Id), wherein:

R5, together with a carbon atom of R1, represents:
a 4-, 5-, 6- or 7-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R5, together with a carbon atom of R1, represents:
a 4-, 5-, 6- or 7-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R6 represents:
either:
a substituent selected from hydrogen or a C₁-C₆-alkyl-, C₁-C₆-haloalkyl-, C₃-C₁₀-cycloalkyl-, C₃-C₁₀-cycloalkyl-C₁-C₆-alkyl-, aryl- optionally substituted one or more times, independently from each other, with an R substituent; heteroaryl-optionally substituted one or more times, independently from each other, with an R substituent; group;
   or:
together with a carbon atom of R1, represents a 4-, 5-, 6- or 7-membered cyclic amine group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
   or:
R5 and R6 together represent a 4-, 5-, or 6- or 7-membered cyclic amide group, which is optionally substituted with a substituent selected from:
   a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R6 represents:
a substituent selected from hydrogen or a C₁-C₆-alkyl-, C₁-C₆-haloalkyl-, C₃-C₁₀-cycloalkyl-, C₃-C₁₀-cycloalkyl-C₁-C₆-alkyl-, aryl- optionally substituted one or more times, independently from each other, with an R substituent; heteroaryl- optionally substituted one or more times, independently from each other, with an R substituent; group.

In a further embodiment of the above-mentioned fourth variant of the first aspect, the invention relates to compounds of formula (Id), wherein:

R6, together with a carbon atom of R1, represents:
a 4-, 5-, 6- or 7-membered cyclic amine group, which is optionally substituted with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group; said 6- or 7-membered cyclic amine group optionally containing one further heteroatom selected from the group consisting of O, N and S.

In a further embodiment of the above-mentioned fourth variant of the first aspect, the invention relates to compounds of formula (Id), wherein:

R5 and R6 together represent:
a 4-, 5-, or 6- or 7-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R7 and R8 represent:
independently from each other, a substituent selected from:
a hydrogen atom, a C₁-C₆-alkyl- group.

In a further embodiment of the above-mentioned fourth variant of the first aspect, the invention relates to compounds of formula (Id), wherein:

together, R7 and R8 together represent:
a 4-, 5-, 6- or 7-membered cyclic amine group, which is optionally substituted with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 6- or 7-membered cyclic amine group optionally containing one further heteroatom selected from the group consisting of O, N and S.

In a further embodiment of the above-mentioned fourth variant of the first aspect, the invention relates to compounds of formula (Id), wherein:

R7 or R8, together with a carbon atom of R1, represent:
a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S.

In a further embodiment of the above-mentioned fourth variant of the first aspect, the invention relates to compounds of formula (Id), wherein:

R1 represents a linear C₁-C₆-alkyl- group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group.

In a further embodiment of the above-mentioned fourth variant of the first aspect, the invention relates to compounds of formula (Id), wherein:

R3 represents a substituent selected from:
—NHR′, C₁-C₆-alkoxy-, C₃-C₆-cycloalkyl-C₁-C₆-alkoxy- group.

In a further embodiment of the above-mentioned fourth variant of the first aspect, the invention relates to compounds of formula (Id), wherein:

R5 represents:
a substituent selected from a C₁-C₆-alkyl-, C₁-C₆-haloalkyl-, C₃-C₁₀-cycloalkyl.

In a further embodiment of the above-mentioned fourth variant of the first aspect, the invention relates to compounds of formula (Id), wherein:

R5, together with a carbon atom of R1, represents:
a 6-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
   said 6-membered cyclic amide group optionally containing one further nitrogen atom.

In a further embodiment of the above-mentioned fourth variant of the first aspect, the invention relates to compounds of formula (Id), wherein:

R6 represents:
a hydrogen atom.

In a further embodiment of the above-mentioned fourth variant of the first aspect, the invention relates to compounds of formula (Id), wherein:

R6, together with a carbon atom of R1, represents:
a 5- or 6-membered cyclic amine group, which is optionally substituted with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
   said 6-membered cyclic amine group optionally containing one further oxygen atom.

In a further embodiment of the above-mentioned fourth variant of the first aspect, the invention relates to compounds of formula (Id), wherein:

R7 and R8 represent independently from each other, a substituent selected from:
a hydrogen atom, a C₁-C₆-alkyl- group.

In a further embodiment of the above-mentioned fourth variant of the first aspect, the invention relates to compounds of formula (Id), wherein:

R represents a C₁-C₆-alkyl- group.

In a further embodiment of the above-mentioned fourth variant of the first aspect, the invention relates to compounds of formula (Id), wherein:

R′ and R″ represent, independently from each other, a substituent selected from:
a C₁-C₆-alkyl-, C₃-C₁₀-cycloalkyl-, C₁-C₆-haloalkyl group.

In a further embodiment of the above-mentioned fourth variant of the first aspect, the invention relates to compounds of formula (Id), wherein.

n represents an integer of 0 or 1.

In a further embodiment of the above-mentioned aspect, the invention relates to compounds of formula (Id), according to any of the above-mentioned embodiments, in the form of or a stereoisomer, a tautomer, an N-oxide, a hydrate, a solvate, or a salt thereof, or a mixture of same.

In accordance with a further aspect, the present invention covers intermediate compounds which are useful in the preparation of compounds of the present invention of general formula (Id), particularly in the method described herein. In particular, the present invention covers compounds of general formula (Ed):

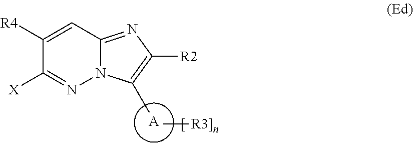

in which A, R2, R3, R4 and n are as defined for the compound of general formula (Id) supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Id), particularly in the method described herein. In particular, the present invention covers compounds of general formula (Fd):

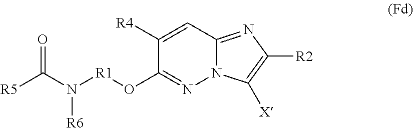

in which R1, R2, R4, R5 and R6 are as defined for the compound of general formula (Id) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Id), particularly in the method described herein. In particular, the present invention covers compounds of general formula (Gd):

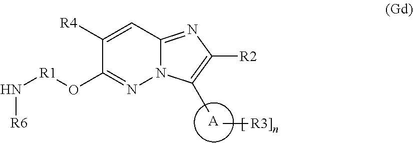

in which A, R1, R2, R3, R4, R6 and n are as defined for the compound of general formula (Id) supra.

In accordance with a further aspect, the present invention covers intermediate compounds which are useful in the preparation of compounds of the present invention of general formula (Id), particularly in the method described herein. In particular, the present invention covers compounds of general formula (Hd):

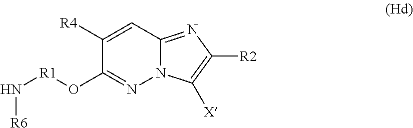

in which R1, R2, R4 and R6 are as defined for the compound of general formula (Id) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Id), particularly in the method described herein. In particular, the present invention covers compounds of general formula (Kd):

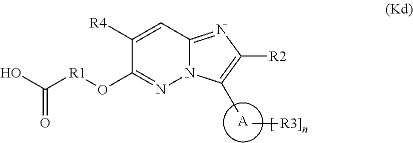

in which A, R1, R2, R3, R4 and n are as defined for the compound of general formula (Id) supra.

In accordance with a further aspect, the present invention covers intermediate compounds which are useful in the preparation of compounds of the present invention of general formula (Id), particularly in the method described herein. In particular, the present invention covers compounds of general formula (Ld):

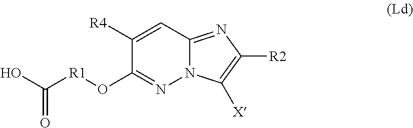

in which R1, R2 and R4 are as defined for the compound of general formula (Id) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with a further aspect, the present invention covers intermediate compounds which are useful in the preparation of compounds of the present invention of general formula (Id), particularly in the method described herein. In particular, the present invention covers compounds of general formula (Md):

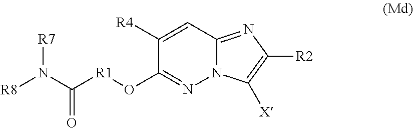

in which R1, R2, R4, R7 and R8 are as defined for the compound of general formula (Id) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

In accordance with yet another aspect, the present invention covers the use of the intermediate compounds of general formula (Ed):

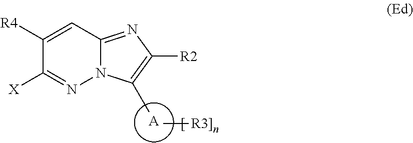

in which A, R2, R3, R4 and n are as defined for the compound of general formula (Id) supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group for example, for the preparation of a compound of general formula (Id) as defined supra.

In accordance with yet another aspect, the present invention covers the use of the intermediate compounds of general formula (Fd):

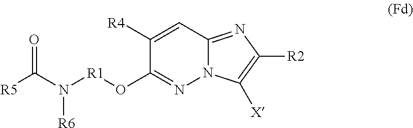

in which R1, R2, R4, R5 and R6 are as defined for the compound of general formula (Id) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Id) as defined supra.

In accordance with yet another aspect, the present invention covers the use of the intermediate compounds of general formula (Gd):

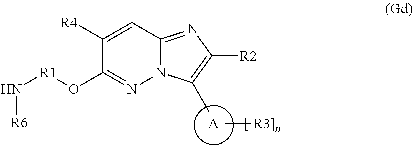

in which A, R1, R2, R3, R4, R6 and n are as defined for the compound of general formula (I) supra, for the preparation of a compound of general formula (Id) as defined supra.

In accordance with yet another aspect, the present invention covers the use of the intermediate compounds of general formula (Hd):

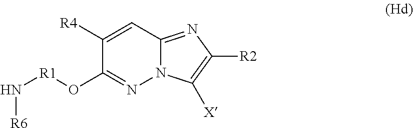

in which R1, R2, R4 and R6 are as defined for the compound of general formula (Id) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Id) as defined supra.

In accordance with yet another aspect, the present invention covers the use of the intermediate compounds of general formula (Kd):

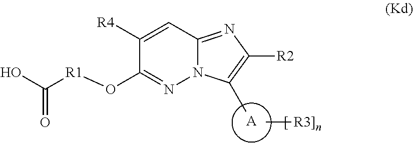

in which A, R1, R2, R3, R4 and n are as defined for the compound of general formula (Id) supra, for the preparation of a compound of general formula (Id) as defined supra.

In accordance with yet another aspect, the present invention covers the use of the intermediate compounds of general formula (Ld):

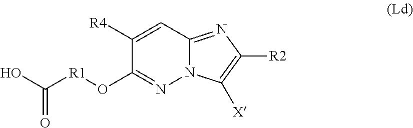

in which R1, R2 and R4 are as defined for the compound of general formula (Id) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Id) as defined supra.

In accordance with yet another aspect, the present invention covers the use of the intermediate compounds of general formula (Md):

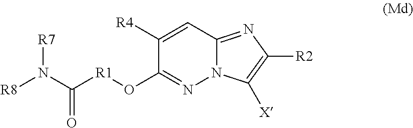

in which R1, R2, R4, R7 and R8 are as defined for the compound of general formula (Id) supra, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, for the preparation of a compound of general formula (Id) as defined supra.

### EXPERIMENTAL SECTION

The following table lists the abbreviations used in this paragraph, and in the examples section.

| Abbreviation | Meaning |
|---|---|
| DMSO | dimethyl sulfoxide |
| THF | Tetrahydrofurane |
| NMR | nuclear magnetic resonance |
| DMF | N,N-dimethylforamide |
| TFA | trifluoroacetic acid |
| MS | mass spectroscopy |
| Rₜ | retention time |
| HPLC, LC | high performance liquid chromatography |
| h | Hour |
| min | Minute |
| COMU | N-[({[(1Z)-1-cyano-2-ethoxy-2- |
| | oxoethylidene]amino}oxy)-(morpholin- |
| | 4-yl)methylidene]-N-methylmethanaminium hexafluoro- |
| | phosphate |
| HATU | N-[(Dimethylamino)-1H-1,2,3-triazolo-[4,5-b]pyridin-1- |
| | ylmethylene]-N-methylmethanaminium |
| | hexafluorophosphate N-oxide |
| PdCl₂(PPh₃)₂ | dichlorobis(triphenylphosphine)palladium(II) |
| BINAP | 1,1′-binaphthalene-2,2′-diylbis(diphenylphosphane) |
| Pd₂dba₃ | (1E,4E)-1,5-diphenylpenta-1,4-dien-3-one—palladium |
| | (3:2) |
| Pddba₂ | (1E,4E)-1,5-diphenylpenta-1,4-dien-3-one—palladium |
| or | (2:1) |
| Pd(dba)₂ | |
| Pd(PPh₃)₄ | Tetrakis(triphenylphosphin)palladium(0), |
| | Palladium—triphenylphosphane (1:4) |
| X-Phos | dicyclohexyl(2′,4′,6′-triisopropylbiphenyl-2-yl)phosphine |
| TBAF | tetrabutylammoniumfluorid |

### Syntheses of Compounds of General Formula (Ia) and (Id) (Overview):

The compounds of general formula (Ia) and (Id) of the present invention can be prepared as described in Section 1.

### Section 1

Scheme 1a and the procedures described below illustrate general synthetic routes to the compounds of general formula (Ia) or (Id) of the invention and are not intended to be limiting. It is clear to the person skilled in the art that the order of transformations as exemplified in Scheme 1a can be modified in various ways. The order of transformations exemplified in the Scheme 1a is therefore not intended to be limiting. In addition, interconversion of any of the substituents, R1, R2, R3, R4 and A, can be achieved before and/or after the exemplified transformations. These modifications can be such as the introduction of protecting groups, cleavage of protecting groups, exchange, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example P. G. M. Wuts and T. W. Greene in “Protective Groups in Organic Synthesis”, 4^{th} edition, Wiley 2006). Specific examples are described in the subsequent paragraphs. Further, it is possible that two or more successive steps may be performed without work-up being performed between said steps, e.g. a “one-pot” reaction, as is well-known to the person skilled in the art.

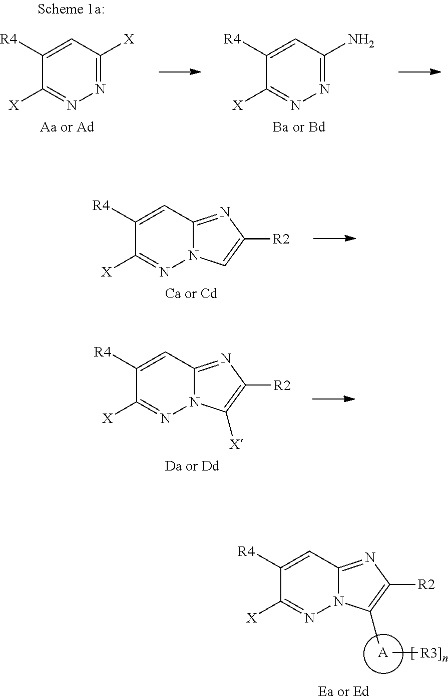

in which A, R1, R2, R3, R4 and n are as defined supra, and X and X′ represent a leaving group, such as a halogen atom, for example a chlorine, bromine or iodine atom, or a perfluoroalkylsulfonate group for example, such as a trifluoromethylsulfonate group, a nonafluorobutylsulfonate group, for example.

In the first step, a compound of formula Aa or Ad, i.e. a dichloropyridazine bearing suitable X substituents, can be reacted with ammonia at elevated temperature and pressure to give a compound of general formula Ba or Bd, respectively. [in analogy to WO200733080, which is hereby incorporated herein in its entirety as reference]

In the second step, a compound of general formula Ba or Bd reacts, for example, with chloroacetaldehyde or bromoacetaldehyde diethylacetal to give the bicyclic ring system Ca or Cd, respectively, [in analogy to DE102006029447, which is hereby incorporated herein in its entirety as reference].

Activation of position 3 of the bicyclic system to give compounds of general formula Da or Dd can be accomplished, for example, by bromination or iodination of compounds of general formula Ca or Cd, respectively, using N-bromo-succinimide or N-iodo-succinimide, respectively.

In the fourth step, introduction of residue A-[R3]ₙ can be achieved using suitably catalyzed cross-coupling reactions employing, for example, boronic acids or stannanes, which results in compounds of general formula Ea or Ed.

Preparation of the examples of the present invention from compounds of general formula Ea Ed can be achieved in a variety of ways for example by the methods described below.

For example, synthesis of the examples can be achieved as outlined in scheme 2a.

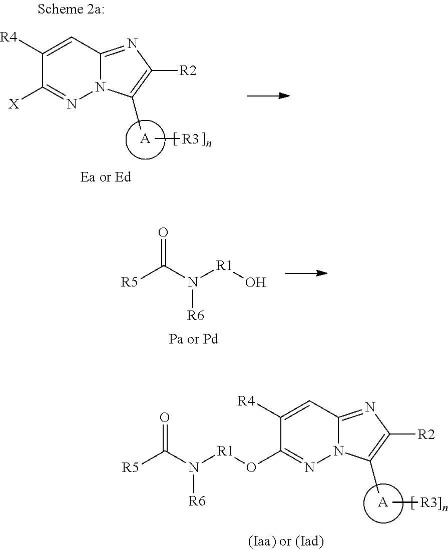

Compounds of general formula Ea or Ed serve as central intermediates for the introduction of various side chains containing an alcohol function, which results in imidazopyridazinyl-ethers of general formula (Iaa) or (Iad), respectively. Introduction of the side chains can be achieved, for example, using an alcohol of general formula Pa or Pd, employing bases such as sodium hydride for example. Depending on the nature of the side chain it may be necessary to run these reactions at elevated temperatures. It may also be necessary to introduce side chains decorated with suitable protecting groups on functional groups which may disturb the desired reaction.

The fourth step, as described in the sequence of Scheme 1a, and the fifth step, as described in Scheme 2a, may also be interconverted as illustrated in Scheme 2a.1.

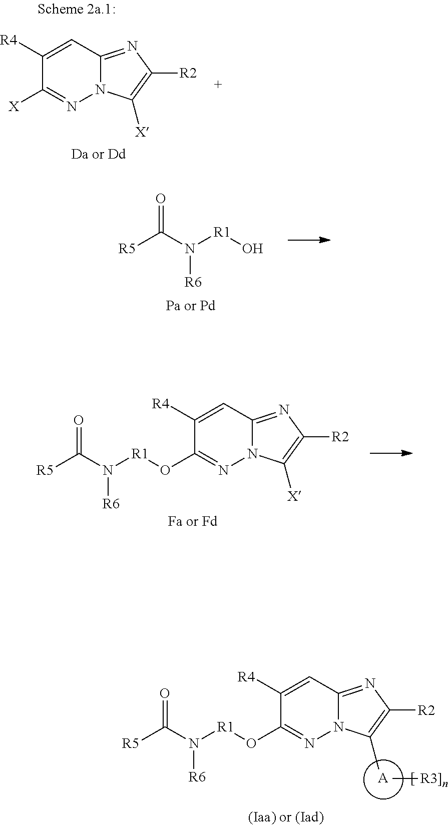

Alternatively, the compounds of the present invention may be synthesized as depicted in scheme 3a:

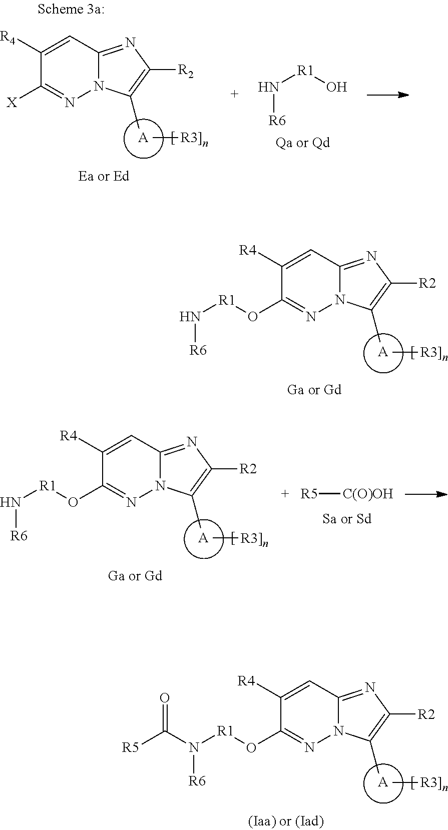

Starting from central intermediate Ea, a suitable amino alcohol side chain may be introduced, for example, using an alcohol of general formula Qa or Qd, employing a base, such as sodium hydride for example, to give an intermediate of general formula Ga or Gd, respectively. Depending on the nature of the amino alcohol, it may be necessary to use suitably protected amino alcohol. For example, the tert-butyl-carbonyloxy as protecting group may be used. Cleavage of this group may be achieved, for example, using trifluoroacetic acid. Intermediate Ga or Gd may then be converted into the final compounds by applying standard amide coupling methods, such as for example use of HATU and a base in presence of a carboxylic acid to generate compounds of general formula Iaa or Iad, respectively. Alternatively, acid chlorides or anhydrides in presence of bases may be used for generation of compounds of general formula Iaa or Iad.

Scheme 3a.1 outlines a variation of this method starting from intermediate Da or Dd. Here, the amino alcohol side chain may be introduced prior to the cross-coupling reaction to give intermediate Ha or Hd, respectively. Introduction of the amide bond in the final products may be achieved prior or after cross-coupling reaction, via intermediates Fa or Fd respectively, or, in turn respectively, Ga or Gd, respectively.

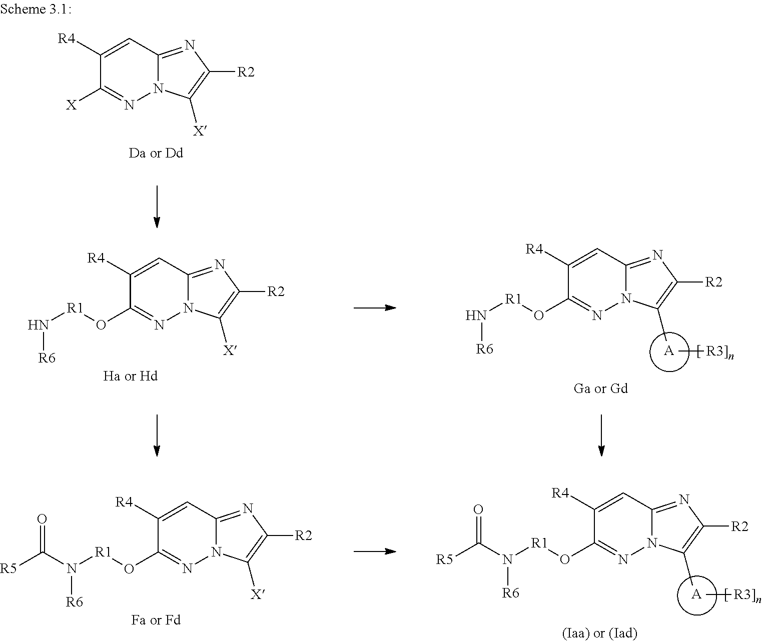

Another alternative synthesis is depicted in scheme 4a.

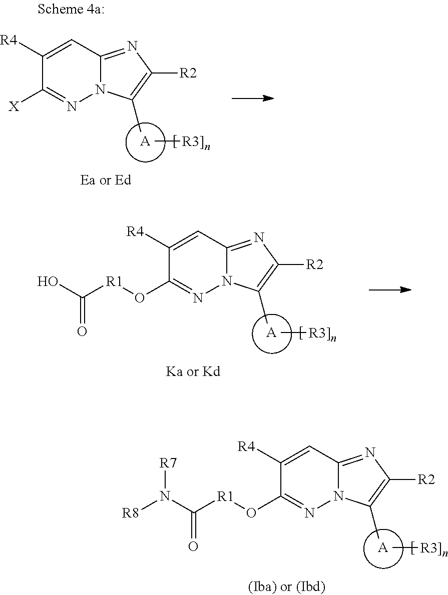

Scheme 4a describes the reaction of intermediate Ea or Ed with a carboxylic acid alcohol moiety in presence of a base to give compounds of general formula Ka or Kd, respectively. It may be necessary, to introduce suitably protected variants of the carboxylic acids, such as esters which may be cleaved before the amide coupling reaction. Compounds of general formula Ka or Kd may be converted by applying standard amide coupling methods, such as for example use of HATU and a base in presence of an amine to generate compounds of general formula Iba or Ibd, respectively. Alternatively, compounds of general formula Ka or Kd may be converted to acid chlorides or anhydrides which may be used in presence of bases for generation of compounds of general formula Iba or Ibd, respectively.

Scheme 4a.1 outlines a variation of this method starting from intermediate Da or Dd. Here, the carboxylic acid alcohol side chain may be introduced prior to the cross-coupling reaction to give intermediate La or Ld, respectively. Introduction of the amide bond in the final products may be achieved prior or after cross-coupling reaction, via intermediates Ka or Kd, respectively, or, in turn respectively, Ma or Md, respectively. Again, depending on the nature of the carboxylic acids employed, protection and deprotection of the acid moiety, for example as esters may be necessary.

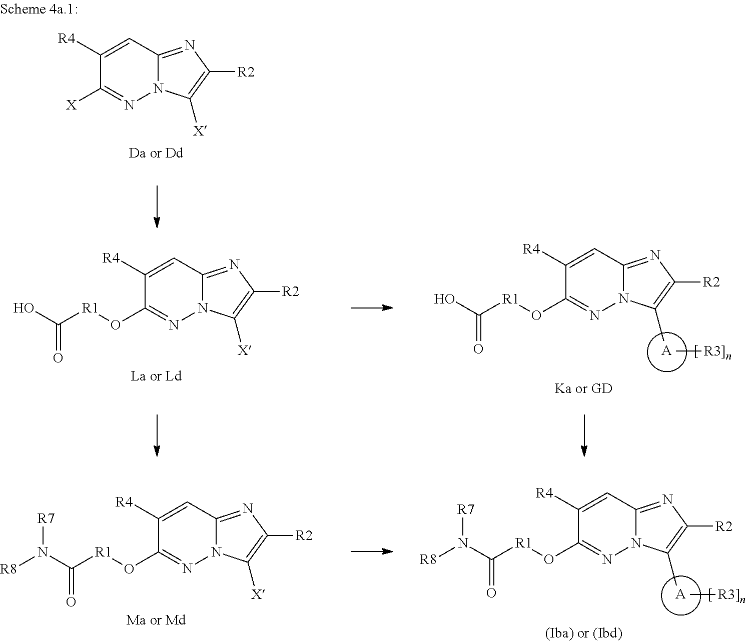

Another alternative synthesis is depicted in scheme 5a.

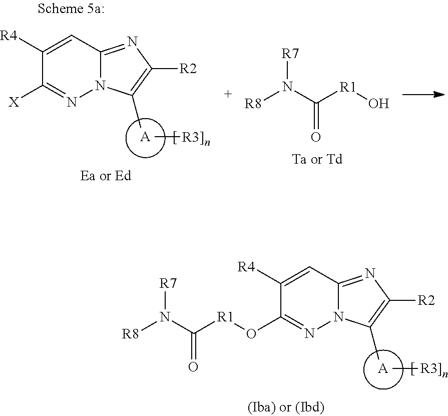

Compounds of general formula Ea or Ed serve as central intermediates for the introduction of various side chains containing an alcohol function, which results in imidazopyridazinyl-ethers of general formula (Iba) or (Ibd), respectively. Introduction of the side chains can be achieved, for example, using an alcohol, employing a base such as for example sodium hydride. Depending on the nature of the side chain it may be necessary to run these reactions at elevated temperatures. It may also be necessary to introduce side chains decorated with suitable protecting groups on functional groups which may disturb the desired reaction.

The fourth step, as described in the sequence of Scheme 1a, and the fifth step, as described in Scheme 5a, may also be interconverted as illustrated in Scheme 5a.1.

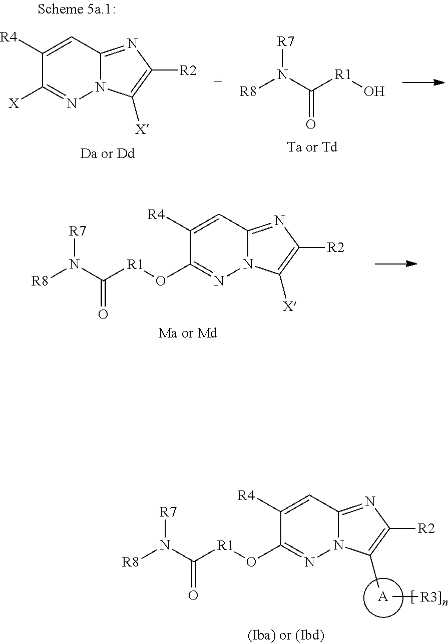

Synthesis of compounds of general formula (Ia) and (Id) of the present invention Compounds of general formula (Iaa) and (Iad) wherein A, R1, R2, R3, R4 and n have the meaning as given for general formula (Ia) or (Id), and wherein Y represents R5-CO—R6N— can be synthesized according to the procedures depicted in Schemes 2a, 2a.1, 3a and 3a.1. These schemes exemplify the main routes that allow variations in A, Y, R1, R2, R3, R4, R5, R6 and n at different stages of the synthesis. However, also other routes may be used to synthesise the target compounds, in accordance with common general knowledge of the person skilled in the art of organic synthesis.

Compounds of general formula (Iba) and (Ibd) wherein A, R1, R2, R3, R4 and n have the meaning as given for general formula (Ia) or (Id), and wherein Y represents R8R7N—CO— can be synthesized according to the procedures depicted in Schemes 4a, 4a.1, 5a and 5a.1. These schemes exemplify the main routes that allow variations in A, Y, R1, R2, R3, R4, R7, R8 and n at different stages of the synthesis. However, also other routes may be used to synthesise the target compounds, in accordance with common general knowledge of the person skilled in the art of organic synthesis.

A method of preparing a compound of general formula (Ia) or (Id) as defined in the claims, said method comprising the step of allowing an intermediate compound of general formula (Ea) or (Ed), respectively:

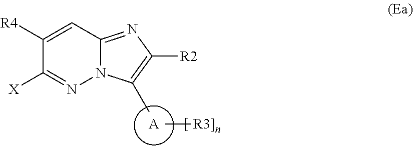

in which A, R2, R3, R4 and n are as defined in the claims for the compound of general formula (Ia) or (Id), respectively, and X represents a leaving group, such as a halogen atom, for example a chlorine, bromine or iodine atom, or a perfluoroalkylsulfonate group for example, such as a trifluoromethylsulfonate group or a nonafluorobutylsulfonate group, for example,
to react with a compound of general formula (IIa) or (IId), respectively:

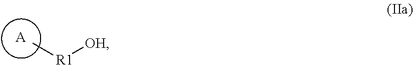

in which R1 and Y are as defined in the claims for the compound of general formula (Ia) or (Id), respectively,
thereby giving a compound of general formula (Ia) or (Id), respectively:

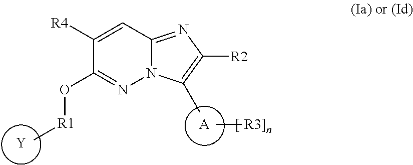

in which A, Y, R1, R2, R3, R4 and n are as defined in the claims for the compound of general formula (Ia) or (Id), respectively.

A method of preparing a compound of general formula (Ia) or (Id) as defined in the claims, said method comprising the step of allowing an intermediate compound of general formula (Fa) or (Fd), respectively:

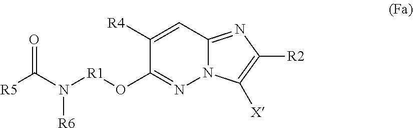

in which R1, R2, R4, R5 and R6 are as defined in the claims for the compound of general formula (Ia) or (Id), respectively, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example,
to react with a compound of general formula (IIIa) or (IIId):

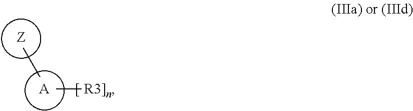

in which A, R3 and n are as defined in the claims for the compound of general formula (Ia) or (Id), and Z represents an activating group suitable for catalyzed cross-coupling reactions, such as a boronic acid or a stannane, for example, such as a tri-n-butylstannyl group, for example,
thereby giving a compound of general formula (Iaa) or (Iad), respectively:

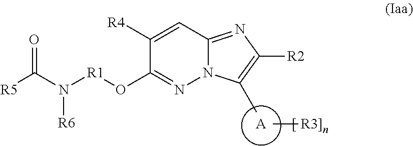

in which A, R1, R2, R3, R4, R5, R6 and n are as defined in the claims for the compound of general formula (Ia) or (Id), respectively.

A method of preparing a compound of general formula (Ia) or (Id) as defined in the claims, said method comprising the step of allowing an intermediate compound of general formula (Ga) or (Gd):

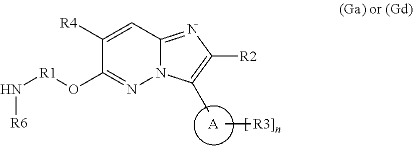

in which A, R1, R2, R3, R4, R6 and n are as defined in the claims for the compound of general formula (Ia) or (Id),
to react with a compound of general formula (IVa) or (IVd):

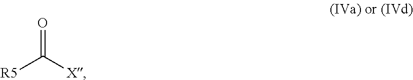

in which R5 is as defined in the claims for the compound of general formula (Ia) or (Id), and in which X″ represents a leaving group, such as a halogen atom, for example a chlorine or fluorine atom, or a carbonyloxy group for example, such as a ethylcarbonyloxy group, for example,
thereby giving a compound of general formula (Iaa) or (Iad), respectively:

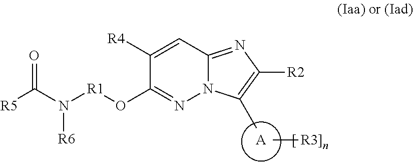

in which A, R1, R2, R3, R4, R5, R6 and n are as defined in the claims for the compound of general formula (Ia) or (Id).

A method of preparing a compound of general formula (Ia) or (Id) as defined in the claims, said method comprising the step of allowing an intermediate compound of general formula (Ha) or (Hd):

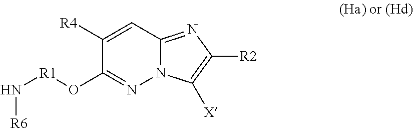

in which R1, R2, R4, and R6 are as defined in the claims for the compound of general formula (Ia) or (Id), and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, to react with a compound of general formula (IVa) or (IVd):

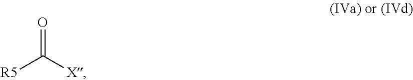

in which R5 is as defined in the claims for the compound of general formula (Ia) or (Id), and in which X″ represents a leaving group, such as a halogen atom, for example a chlorine or fluorine atom, or a carbonyloxy group for example, such as a ethylcarbonyloxy group, for example,
thereby giving a compound of general formula (Fa) or (Fd), respectively:

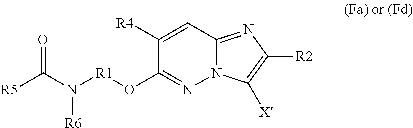

in which R1, R2, R4, R5 and R6 are as defined in the claims for the compound of general formula (Ia) or (Id), and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example.

A method of preparing a compound of general formula (Ia) or (Id) as defined in the claims, said method comprising the step of allowing an intermediate compound of general formula (Ka) or (Kd):

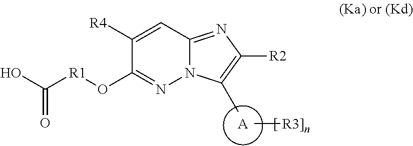

in which A, R1, R2, R3, R4 and n are as defined as defined in the claims for the compound of general formula (Ia) of (Id),
to react with a compound of general formula (Va) or (Vd):

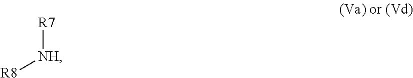

in which R7 and R8 are as defined in the claims for the compound of general formula (Ia) or (Id),
thereby giving a compound of general formula (Iba) or (Ibd), respectively:

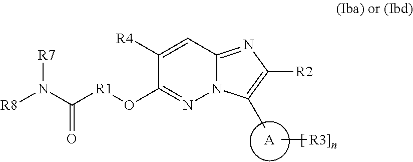

in which A, R1, R2, R3, R4, R7, R8 and n are as defined in the claims for the compound of general formula (Ia) or (Id).

A method of preparing a compound of general formula (Ia) or (Id) as defined in the claims, said method comprising the step of allowing an intermediate compound of general formula (La) or (Ld):

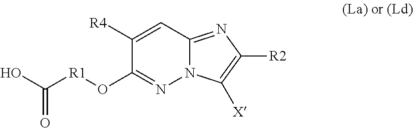

in which R1, R2 and R4 are as defined in the claims for the compound of general formula (Ia) or (Id), and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, to react with a compound of general formula (Va) or (vd):

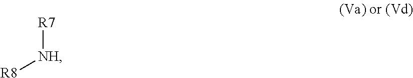

in which R7 and R8 are as defined in the claims for the compound of general formula (Ia) or (Id), respectively,
thereby giving a compound of general formula (Ma) or (Md), respectively:

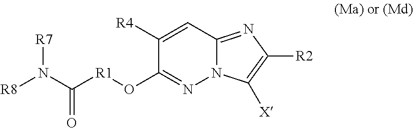

in which R1, R2, R4, R7, R8 and X′ are as defined in the claims for the compound of general formula (Ia) or (Id), respectively.

A method of preparing a compound of general formula (Ia) or (Id) as defined in the claims, said method comprising the step of allowing an intermediate compound of general formula (Ma) or (Md):

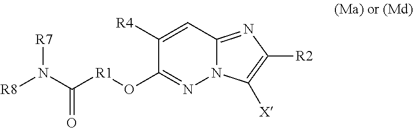

in which R1, R2, R4, R7 and R8 are as defined in the claims for the compound of general formula (Ia) or (Id) respectively, and in which X′ represents a leaving group, such as a halogen atom, for example a chlorine, bromine or iodine atom, or a perfluoroalkylsulfonate group for example, such as a trifluoromethylsulfonate group or a nonafluorobutylsulfonate group, for example,
to react with a compound of general formula (IIIa) or (IIId), respectively:

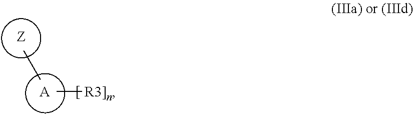

in which A, R3 and n are as defined in the claims for the compound of general formula (Ia) or (Id) respectively, and Z represents an activating group suitable for catalyzed cross-coupling reactions, such as a boronic acid or a stannane, for example, such as a tri-n-butylstannyl group, for example,
thereby giving a compound of general formula (Iba) or (Ibd), respectively:

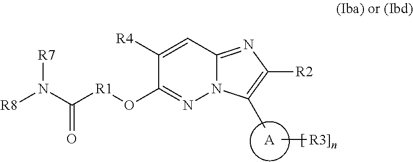

in which A, R1, R2, R3, R4, R7, R8 and n are as defined in the claims for the compound of general formula (Ia) or (Id).

### Syntheses of Compounds of General Formula (Ib) and (Ic) (Overview):

The compounds of general formula (Ib) and (Ic) of the present invention can be prepared as described in Section 2.

### Section 2

Scheme 1b and the procedures described below illustrate general synthetic routes to the compounds of general formula (Ib) or (Ic) of the invention and are not intended to be limiting. It is clear to the person skilled in the art that the order of transformations as exemplified in Scheme 1b can be modified in various ways. The order of transformations exemplified in the Scheme 1b is therefore not intended to be limiting. In addition, interconversion of any of the substituents, R1, R2, R3, R4 and A, can be achieved before and/or after the exemplified transformations. These modifications can be such as the introduction of protecting groups, cleavage of protecting groups, exchange, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example P. G. M. Wuts and T. W. Greene in “Protective Groups in Organic Synthesis”, 4^{th} edition, Wiley 2006). Specific examples are described in the subsequent paragraphs. Further, it is possible that two or more successive steps may be performed without work-up being performed between said steps, e.g. a “one-pot” reaction, as is well-known to the person skilled in the art.

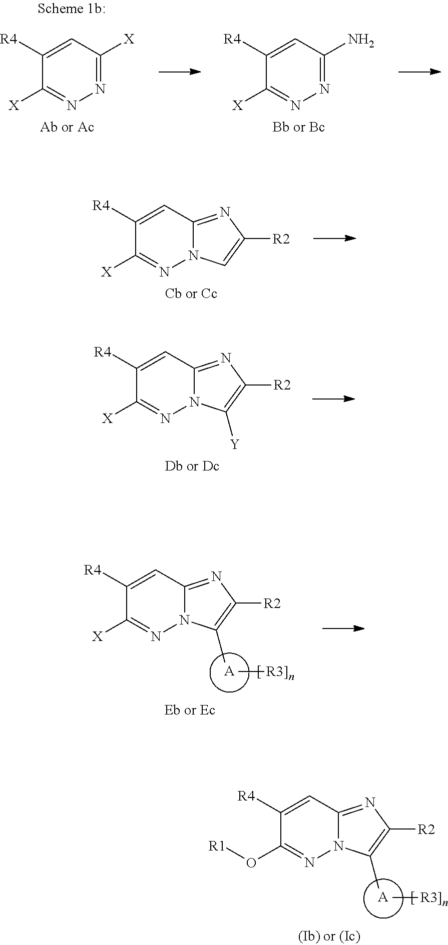

in which A, R1, R2, R3, R4 and n are as defined supra, and in which X and Y represent a leaving group, such as a halogen atom, for example a chlorine, bromine or iodine atom, or a perfluoroalkylsulfonate group for example, such as a trifluoromethylsulfonate group, a nonafluorobutylsulfonate group, for example.

In the first step, a compound of formula Ab (or Ac), bearing suitable X substituents, i.e. a dichloropyridazine, can be reacted with ammonia at elevated temperature and pressure to give a compound of general formula Bb (or Bc). [in analogy to WO200733080, which is hereby incorporated herein in its entirety as reference]

In the second step, a compound of general formula Bb (or Bc) reacts, for example, with chloroacetaldehyde or bromoacetaldehyde diacetal to give the bicyclic ring system Cb (or Cc) [in analogy to DE102006029447, which is hereby incorporated herein in its entirety as reference].

Activation of position 3 of the bicyclic system to give compounds of general formula Db (or Dc) can be accomplished, for example, by bromination or iodination of compounds of general formula Cb (or Cc) using N-bromo-succinimide or N-iodo-succinimide, respectively.

In the fourth step, introduction of residue A-[R3]ₙ can be achieved using suitably catalyzed cross-coupling reactions employing, for example, boronic acids or stannanes, which results in compounds of general formula Eb (or Ec).

Compounds of general formula Eb (or Ec) serve as central intermediates for the introduction of various side chains containing an alcohol function, which results in imidazopyridazinyl-ethers of general formula (Ib) or (Ic). Introduction of the side chains can be achieved, for example, by employing bases such as sodium hydride. Depending on the nature of the side chain it may be necessary to run these reactions at elevated temperatures. It may also be necessary to introduce side chains decorated with suitable protecting groups on functional groups which may disturb the desired reaction.

The fourth and the fifth step of the described sequence may also be interconverted as illustrated in Scheme 2b.

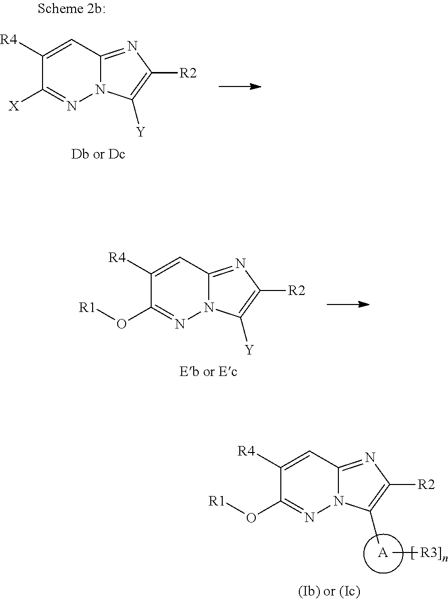

in which A, R1, R2, R3, R4 and n are as defined supra, and in which X and Y represent a leaving group, such as a halogen atom, for example a chlorine, bromine or iodine atom, or a perfluoroalkylsulfonate group for example, such as a trifluoromethylsulfonate group, a nonafluorobutylsulfonate group, for example.

The residues A-[R3]ₙ may be prepared, for example, as depicted in Scheme 3b.

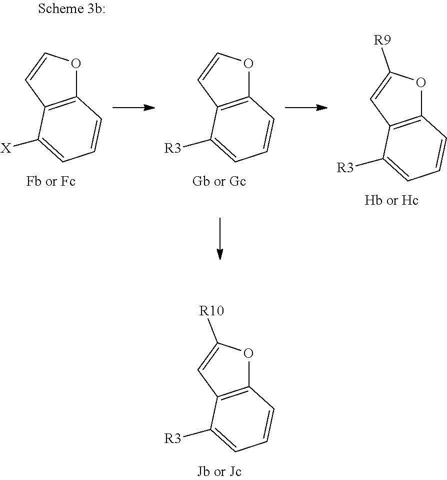

in which R3 is as defined supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group for example, and R9 represents a boronic acid —B(OH)₂, or a boronic acid ester, and R10 represents a stannyl group, such as a tri-n-butylstannyl group for example.

Starting from a benzofuran Fb (or Fc) which carries a halogen atom, for example a bromine atom, or another suitably funtionalized leaving group, such as a trifluoromethylsulfonate group or a nonafluorobutylsulfonate group, it is possible to introduce amines, for example, by using Pd-catalyzed methods [see for example WO2012036253 or Bioorganic & Medicinal Chemistry, 2010, volume 18, pages 7593-7606], which results in compounds of general formula Gb (or Gc). Depending on the nature of R3, the R3 moiety can be modified by means of alkylation, acylation, oxidation, reduction, and the like, prior to the next step; and, depending on the nature of R3, protecting group operations may be necessary prior to the next step. Following the introduction of the R3 moiety, or following the modification of the R3 moiety, it is possible to activate the 2-position of the benzofuran for the cross coupling reactions as employed in scheme 1b or 2b, after deprotonation with strong bases, such as butyl lithium for example, and reaction with trialkylborates, such as triisopropyl borate for example, or with bis(pinacolato)diboron for example [see, for example WO2009154780 or ACS Medicinal Chemistry Letters, 2011, volume 2, page 97], to give the compounds of general formula Hb (or Hc).

Alternatively, the compounds of general formula Gb (or Gc) after deprotonation with strong bases, such as butyl lithium for example, can be reacted with trialkyltinhalides, such as tributyl tin chloride for example [see, for example, Bioorganic & Medicinal Chemistry, 2012, volume 20, pages 2762-2772], to give the corresponding stannylbenzofuranes of general formula Jb (or Jc), which are also suitable for the cross coupling reactions as employed in scheme 1b or 2b.

In accordance with an embodiment, the present invention also relates to a method of preparing a compound of general formula (I) supra, said method comprising the step of allowing an intermediate compound of general formula (Eb) or (Ec):

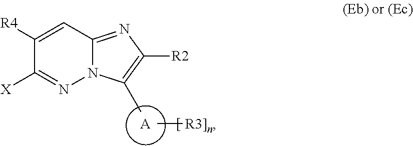

in which A, R2, R3, R4 and n are as defined for the compound of general formula (Ib) or (Ic), respectively, supra, and in which X represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, to react with a compound of general formula (IIb) or (IIc):

R1-OH (IIb) or (IIc),

in which R1 is as defined for the compound of general formula (Ib) or (Ic), respectively, supra,
thereby giving a compound of general formula (Ib) or (Ic):

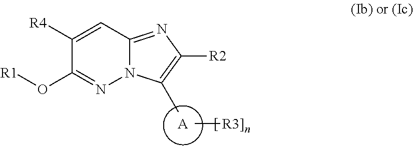

in which A, R1, R2, R3, R4 and n are as defined for the compound of general formula (Ib) or (Ic), respectively, supra.

In accordance with an embodiment, the present invention also relates also to a method of preparing a compound of general formula (Ib) or (Ic) supra, said method comprising the step of allowing an intermediate compound of general formula (Eb′) or (Ec′):

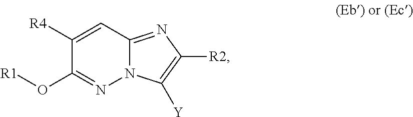

in which R1, R2 and R4 are as defined for the compound of general formula (Ib) or (Ic), respectively, supra, and in which Y represents a leaving group, such as a halogen atom, for example a chlorine, bromine or iodine atom, or a perfluoroalkylsulfonate group for example, such as a trifluoromethylsulfonate group or a nonafluorobutylsulfonate group, for example, to react with a compound of general formula (IIIb) or (IIIc):

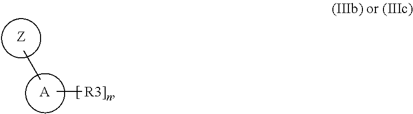

in which A, R3 and n are as defined for the compound of general formula (Ib) or (Ic), respectively, supra, and in which Z represents an activating group suitable for catalyzed cross-coupling reactions, such as a boronic acid —B(OH)₂, or a boronic acid ester, or a stannyl group, for example, such as a tri-n-butylstannyl group, for example, thereby giving a compound of general formula (Ib) or (Ic):

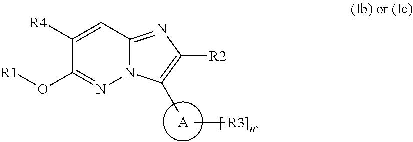

in which A, R1, R2, R3, R4 and n are as defined for the compound of general formula (Ib) or (Ic), respectively, supra.

### General Part

Chemical names were generated using ACD/Name Batch Version 12.01.

All reagents, for which the synthesis is not described in the experimental part, are either commercially available or synthesized as described in literature references.

### HPLC Methods:

### Method 1:

Instrument: Waters Acquity UPLCMS ZQ4000; Column: Acquity UPLC BEH C18 1.7 μm, 50×2.1 mm; eluent A: water+0.05 vol % formic acid, Eluent B: acetonitrile+0.05 vol % formic acid gradient: 0-1.6 min 1-99% B, 1.6-2.0 min 99% B; flow 0.8 mL/min; temperature: 60° C.; injection: 2 μL; DAD scan: 210-400 nm; ELSD

### Method 2:

Instrument: Waters Acquity UPLCMS SQD 3001; Column: Acquity UPLC BEH C18 1.7 μm, 50×2.1 mm; eluent A: water+0.1 vol % formic acid, eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD

### Method 3:

Instrument: Waters Acquity UPLCMS SQD; Column: Acquity UPLC BEH C18 1.7 μm, 50×2.1 mm; eluent A: water+0.05 vol % formic acid (95%), eluent B: acetonitrile+0.05 vol % formic acid (95%), gradient: 0-1.6 min 1-99% B, 1.6-2.0 min 99% B; flow 0.8 mL/min; temperature: 60° C.; injection: 2 μL; DAD scan: 210-400 nm; ELSD

### Method 4:

Instrument MS: Waters ZQ; Instrument HPLC: Waters UPLC Acquity; Column: Acquity BEH C18 (Waters), 50 mm×2.1 mm, 1.71 μm; eluent A: water+0.1 vol % formic acid, eluent B: acetonitrile (Lichrosolv Merck); gradient: 0.0 min 99% vol A-1.6 min 1 vol % A-1.8 min 1 vol % A-1.81 min 99 vol % A-2.0 min 99 vol % A; temperature: 60° C.; flow: 0.8 mL/min; UV-Detection PDA 210-400 nm

### Method 5:

Instrument: Waters Acquity UPLC-MS SQD; Column: Acquity UPLC BEH C18 1.7 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; injection: 2 μL; DAD scan: 210-400 nm; ELSD.

### Method 6:

Instrument: Waters Acquity UPLCMS SQD 3001; Column: Acquity UPLC BEH C18 1.7 μm, 50×2.1 mm; eluent A: water+0.1 vol % formic acid (95%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD

### Method 7:

Instrument: Waters Acquity UPLCMS SQD; Column: Acquity UPLC BEH C18 1.7 μm, 50×2.1 mm; eluent A: water+0.05 vol % formic acid (95%), eluent B: acetonitrile+0.05 vol % formic acid (95%), gradient: 0-1.6 min 1-99% B, 1.6-2.0 min 99% B; flow 0.8 mL/min; temperature: 60° C.; injection: 2 μL; DAD scan: 210-400 nm; ELSD

### Method 8:

Instrument: Waters Acquity UPLC-MS SQD; Column: Acquity UPLC BEH C18 1.7 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; injection: 2 μL; DAD scan: 210-400 nm; ELSD.

### Method 9:

Instrument: Waters Acquity UPLCMS SQD 3001; Column: Acquity UPLC BEH C18 1.7 μm, 50×2.1 mm; eluent A: water+0.2 vol. % ammonia (32%), eluent B: acetonitrile, gradient: 0-1.6 min 1-99% B, 1.6-2.0 min 99% B; flow 0.8 mL/min; temperature: 60 C; injection: 2 μL; DAD scan: 210-400 nm; ELSD

### Method 10:

Instrument: Waters Acquity UPLCMS SQD; Column: Acquity UPLC BEH C18 1.7 μm, 50×2.1 mm; Eluent A: water+0.2% vol. ammonia (32%), eluent B: acetonitrile; gradient: 0-1.6 min 1-99% B, 1.6-2.0 min 99% B; flow 0.8 ml/min; temperature: 60° C.; injection: 2 al; DAD scan: 210-400 nm; ELSD.

### Method 11:

Instrument: Waters Acquity UPLCMS ZQ4000; Column: Acquity UPLC BEH C18 1.7 μm, 50×2.1 mm; eluent A: water+0.05 vol % formic acid, Eluent B: acetonitrile+0.05 vol % formic acid gradient: 0-1.6 min 1-99% B, 1.6-2.0 min 99% B; flow 0.8 mL/min; temperature: 60° C.; injection: 2 μL; DAD scan: 210-400 nm; ELSD

### Method 12:

Instrument: Waters Acquity UPLCMS SQD 3001; Column: Acquity UPLC BEH C18 1.7 μm, 50×2.1 mm; eluent A: water+0.1 vol % formic acid (95%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD

### Method 13:

Instrument: Waters Acquity UPLCMS SQD; Column: Acquity UPLC BEH C18 1.7 μm, 50×2.1 mm; eluent A: water+0.05 vol % formic acid (95%), eluent B: acetonitrile+0.05 vol % formic acid (95%), gradient: 0-1.6 min 1-99% B, 1.6-2.0 min 99% B; flow 0.8 mL/min; temperature: 60° C.; injection: 2 μL; DAD scan: 210-400 nm; ELSD

### Method 14:

Instrument: Waters Acquity UPLC-MS SQD; Column: Acquity UPLC BEH C18 1.7 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; injection: 2 μL; DAD scan: 210-400 nm; ELSD.

### Method 15:

Instrument: Waters Acquity UPLCMS SQD 3001; Column: Acquity UPLC BEH C18 1.7 μm, 50×2.1 mm; eluent A: water+0.2 vol. % ammonia (32%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD

### Method 16

Instrument: Waters Acquity UPLC-MS SQD; Column: Acquity UPLC BEH C18 1.7 50×2.1 mm; eluent A: water+0.2% vol. ammonia (32%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

### Method 17

Instrument: Waters Acquity UPLC-MS ZQ; column: Acquity UPLC BEH C18 1.7 50×2.1 mm; eluent A: water+0.1% vol. formic acid (99%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

### Method 18:

Instrument: Waters Acquity UPLCMS SQD; Column: Acquity UPLC BEH C18 1.7 μm, 50×2.1 mm; Eluent A: water+0.2% vol. ammonia (32%), eluent B: acetonitrile; gradient: 0-1.6 min 1-99% B, 1.6-2.0 min 99% B; flow 0.8 mL/min; temperature: 60° C.; injection: 2 μl; DAD scan: 210-400 nm; ELSD.

### Method 19:

Instrument: Waters Acquity UPLCMS ZQ4000; Column: Acquity UPLC BEH C18 1.7 μm, 50×2.1 mm; eluent A: water+0.05 vol % formic acid, Eluent B: acetonitrile+0.05 vol % formic acid gradient: 0-1.6 min 1-99% B, 1.6-2.0 min 99% B; flow 0.8 mL/min; temperature: 60° C.; injection: 2 μL; DAD scan: 210-400 nm; ELSD

### Method 20:

Instrument: Waters Acquity UPLCMS SQD 3001; Column: Acquity UPLC BEH C18 1.7 μm, 50×2.1 mm; eluent A: water+0.1 vol % formic acid (95%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD

### Method 21:

Instrument: Waters Acquity UPLCMS SQD; Column: Acquity UPLC BEH C18 1.7 μm, 50×2.1 mm; eluent A: water+0.05 vol % formic acid (95%), eluent B: acetonitrile+0.05 vol % formic acid (95%), gradient: 0-1.6 min 1-99% B, 1.6-2.0 min 99% B; flow 0.8 mL/min; temperature: 60° C.; injection: 2 μL; DAD scan: 210-400 nm; ELSD

### Method 22

Instrument: Waters Acquity UPLC-MS SQD; Column: Acquity UPLC BEH C18 1.7 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; injection: 2 μL; DAD scan: 210-400 nm; ELSD.

### Method 23

Instrument: Waters Acquity UPLCMS SQD 3001; Column: Acquity UPLC BEH C18 1.7 μm, 50×2.1 mm; eluent A: water+0.2 vol. % ammonia (32%), eluent B: acetonitrile, gradient: 0-1.6 min 1-99% B, 1.6-2.0 min 99% B; flow 0.8 mL/min; temperature: 60° C.; injection: 2 μL; DAD scan: 210-400 nm; ELSD.

### INTERMEDIATES

### Intermediate I-1

### 3-Bromo-6-chloro-imidazo[1,2-b]pyridazine

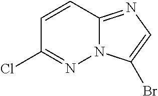 [SMILES: Clc1ccc2ncc(Br)n2n1]

3-Bromo-6-chloro-imidazo[1,2-b]pyridazine was synthesized as described for example in WO 2007/147646 or DE 10 2006 029447, e.g. as follows:

### Step 1: Preparation of 6-Chloroimidazo[1,2-b]pyridazine

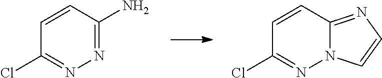 [SMILES: Nc1ccc(Cl)nn1 ; Clc1ccc2nccn2n1]

5.0 g (38.6 mmol) of 3-amino-6-chloropyridazine were heated together with 4.7 mL (40 mmol) of chloroacetaldehyde (55% strength in water) in 15 mL of n-butanol at 120° C. for a period of 5 days. After the reaction was complete, the reaction mixture was added to saturated sodium bicarbonate solution and extracted three times with ethyl acetate. The combined organic phases were then washed with sat. sodium chloride solution and dried over sodium sulfate, and the solvent was removed in vacuo. In the final purification by chromatography on silica gel, 4.17 g (70%) of the desired product were isolated in the form of an amorphous white solid.

¹H-NMR (CHLOROFORM-d): δ [ppm]=7.06 (d, 1H); 7.79 (d, 1H); 7.92, (d, 1H); 7.96 (d, 1H).

### Step 2: Preparation of 3-Bromo-6-chloroimidazo[1,2-b]pyridazine

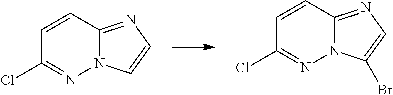 [SMILES: Clc1ccc2nccn2n1 ; Clc1ccc2ncc(Br)n2n1]

478 mg (3.11 mmol) of 6-chloroimidazo[1,2-b]pyridazine were introduced into 10 mL of chloroform under argon and, while cooling in ice, 664 mg (3.73 mmol) of N-bromo-succinimide were added. After the addition was complete, the reaction mixture was stirred at room temperature overnight. The reaction mixture was then mixed with water and ethyl acetate and, after addition of saturated sodium bicarbonate solution, the phases were separated. The aqueous phase was extracted three more times with ethyl acetate. The combined organic phases were then washed with saturated sodium chloride solution and dried over sodium sulfate. In the final removal of the solvent in vacuo, the desired product was isolated in quantitative yield in the form of an amorphous white solid which was employed without further chromatographic purification in subsequent reactions.

¹H-NMR (CHLOROFORM-d): δ [ppm]=7.12 (d, 1H); 7.79 (s, 1H); 7.90, (d, 1H).

### Intermediate I-2

### 3-(1-Benzofur-2-yl)-6-chloroimidazo[1,2-b]pyridazine

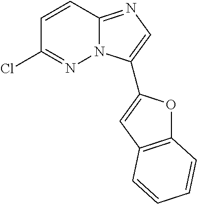 [SMILES: Clc1ccc2ncc(-c3cc4ccccc4o3)n2n1]

13.9 g (59.8 mmol) 3-bromo-6-chloro-imidazo[1,2-b]pyridazine were suspended in 508 mL 1,4-dioxane. 10.1 g (62.8 mmol) 2-benzofuranylboronic acid, 2.76 g (2.29 mmol) tetrakis(triphenylphosphino)palladium-(0) and 19.0 g (179 mmol) sodium carbonate were added. The obtained mixture was heated to 100° C. for 24 h.

400 mL of a saturated aqueous ammonium chloride solution were added. The obtained mixture was extracted with ethyl acetate. The combined organic layers were washed with brine and dried over magnesium sulfate. After evaporation of the solvent, the obtained solid material was digested in 40 mL of a mixture of dichloromethane and methanol (8:2), filtered off and dried in vacuo to yield 5.42 g (44%) of the title compound as solid material.

¹H-NMR (300 MHz, DMSO-d₆): δ [ppm]=7.23-7.40 (2H), 7.51 (1H), 7.59-7.67 (2H), 7.77 (1H), 8.33-8.40 (2H).

LCMS (Method 1): Rₜ=1.35 min; MS (ESIpos) m/z=270 [M+H]⁺.

### Intermediate I-3

### 6-Chloro-3-(4-methoxy-1-benzofuran-2-yl)imidazo[1,2-b]pyridazine

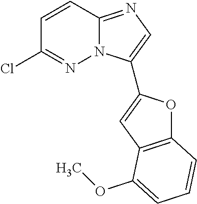 [SMILES: COc1cccc2oc(-c3cnc4ccc(Cl)nn34)cc12]

6-Chloro-3-(4-methoxy-1-benzofuran-2-yl)imidazo[1,2-b]pyridazine was prepared in analogy to 3-(1-benzofur-2-yl)-6-chloroimidazo[1,2-b]pyridazine starting from 1.68 g (7.22 mmol) of 3-bromo-6-chloro-imidazo[1,2-b]pyridazine to yield 43% of a solid material.

¹H-NMR (300 MHz, DMSO-d₆), δ [ppm]=3.96 (3H), 6.85-6.91 (1H), 7.25-7.38 (2H), 7.52-7.59 (2H), 8.37-8.43 (2H).

LCMS (Method 1): Rₜ=1.31 min; MS (ESIpos) m/z=300 [M+H]⁺.

### Intermediate I-4

### 6-Chloro-3-(5-methoxy-1-benzofuran-2-yl)imidazo[1,2-b]pyridazine

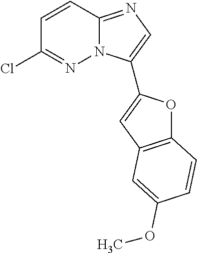 [SMILES: COc1ccc2oc(-c3cnc4ccc(Cl)nn34)cc2c1]

6-Chloro-3-(5-methoxy-1-benzofuran-2-yl)imidazo[1,2-b]pyridazine was prepared in analogy to 3-(1-benzofur-2-yl)-6-chloroimidazo[1,2-b]pyridazine starting from 1.74 g (7.5 mmol) of 3-bromo-6-chloro-imidazo[1,2-b]pyridazine to yield 45% of a solid material.

¹H-NMR (300 MHz, DMSO-d₆), δ [ppm]=3.81 (3H), 6.91-6.99 (1H), 7.33 (1H), 7.50-7.60 (3H), 8.35-8.42 (2H).

LCMS (Method 1): Rₜ=1.29 min; MS (ESIpos) m/z=300 [M+H]⁺.

### Intermediate I-5

### 6-Chloro-3-(5-chloro-1-benzofuran-2-yl)imidazo[1,2-b]pyridazine

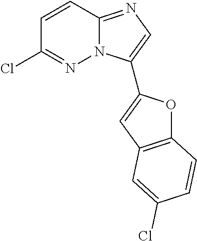 [SMILES: Clc1ccc2oc(-c3cnc4ccc(Cl)nn34)cc2c1]

A mixture of 2.0 g (13.1 mmol) 5-chlorobenzofuran in anhydrous THF (100 mL) was cooled to −78° C. 7.9 mL (19.7 mmol) of a 2.5 M solution of n-butyllithium in hexane was added and the resulting mixture stirred for 1 h at −78° C. 5.3 mL (19.7 mmol) of tributyltin chloride was added. The reaction was stirred at room temperature over night.

Methanol was carefully added and the solvent evaporated. The obtained residue was purified by flash chromatography to yield 6.2 g of crude product of the corresponding 2-stannylbenzofurane, which was used without further purification.

In an inert atmosphere, 2.35 g (10.1 mmol) of 3-bromo-6-chloro-imidazo[1,2-b]pyridazine, 5.8 g (13.1 mmol) of the crude 2-stannylbenzofurane, 192 mg (1.0 mmol) copper (I) iodide and 354 mg (0.5 mmol) bis(triphenylphosphine) palladium(II)chloride in 100 mL of anhydrous THF is stirred over night at 80° C. The solvent was evaporated, the obtained solid was digested in methanol and filtered off to yield 2.73 g of a solid material which was used as crude product.

LCMS (Method 3): Rₜ=1.49 min; MS (ESIpos) m/z=304 [M+H]⁺.

### Intermediate I-6

### 3-(1-Benzofuran-2-yl)-6-{2-[(2S)-pyrrolidin-2-yl]ethoxy}imidazo[1,2-b]-pyridazine

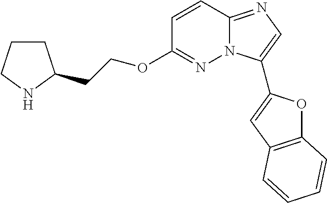 [SMILES: c1ccc2oc(-c3cnc4ccc(OCC[C@@H]5CCCN5)nn34)cc2c1]

Step 1: To 9.3 g (40.4 mmol) [(2S)-1-(tert.-butoxycarbonyl)pyrrolidin-2-yl]acetic acid in 116 mL tetrahydrofurane were added dropwise 40 mL of borane-dimethyl sulfide complex. The resulting mixture was stirred for 2 h at 80° C.

The mixture was carefully poured into saturated aqueous sodium hydrogencarbonate solution. The aqueous layer was extracted with methyl-tert.-butylether. The combined organic layers were washed with brine, dried over magnesium sulfate, and concentrated to give 6.2 g of a crude product which was used without further purification in step 2.

Step 2: In an ice bath, 1.37 g (6.39 mmol) of the crude product from step 1 were added to 224 mg (5.62 mmol) sodium hydride (60% in mineral oil) in 34 mL anhydrous tetrahydrofurane. After 15 min of stirring in the ice bath, 861 mg (3.19 mmol) 3-(1-benzofur-2-yl)-6-chloroimidazo[1,2-b]pyridazine were added. The ice bath was removed and the reaction mixture was stirred for 24 h at room temperature.

The reaction mixture was poured into saturated aqueous ammoniumchloride solution, and extracted with ethyl acetate. The combined organic phases were washed with brine, dried over magnesium sulfate, and concentrated. The obtained crude product (2.1 g) was used without further purification in step 3.

Step 3: To 1.4 g of the crude product from step 2 in 28 mL dichloromethane were added 4.9 mL of trifluoroacetic acid. The mixture was stirred for 1 h. Aqueous sodium hydroxide solution was added until the mixture reached basic pH. Brine was added and the mixture extracted with dichloromethane. The organic layer was separated, dried over magnesium sulfate and concentrated.

The residue was purified by HPLC to give 725 mg of the product as solid material.

¹H-NMR (300 MHz, DMSO-d₆), δ [ppm]=1.57-1.72 (1H), 1.77-2.01 (2H), 2.11-2.32 (3H), 3.09-3.24 (2H), 3.64 (1H), 4.51-4.70 (2H), 7.02 (1H), 7.24-7.37 (2H), 7.60-7.66 (2H), 7.67-7.74 (1H), 8.13-8.23 (2H).

LC-MS (Method 1): Rₜ=0.82 min; MS (ESIpos) m/z=349 [M+H]⁺.

### Intermediate I-7

### 2-{[3-(1-Benzofuran-2-yl)imidazo[1,2-b]pyridazin-6-yl]oxy}ethanamine

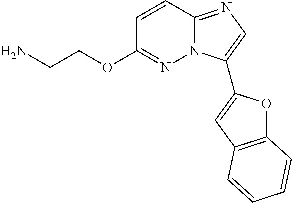 [SMILES: NCCOc1ccc2ncc(-c3cc4ccccc4o3)n2n1]

In an ice bath, 10.4 mg (0.261 mmol) sodium hydride (60% dispersion in mineral oil) were dispensed in 2 mL of anhydrous THF. 18.5 mg (0.297 mmol) 2-aminoethan-1-ol were slowly added. After complete addition, stirring at 0° C. was continued for 15 min. 40.0 mg (0.148 mmol) of 3-(1-benzofur-2-yl)-6-chloroimidazo[1,2-b]pyridazine were added, the ice bath was removed and the resulting mixture was stirred for 17 h at rt.

The reaction mixture was carefully poured into a saturated aqueous ammonium chloride solution. The aqueous layer was extracted with ethyl acetate/methanol (9:1). The combined organic layers were dried over magnesium sulfate, and concentrated.

The crude product (90 mg) was dissolved in dichloromethane, a trace of methanol was added. The mixture was extracted with water, dried over magnesium sulfate, and concentrated to give 45 mg of the title compound as a solid material.

¹H-NMR (300 MHz, DMSO-d₆), δ [ppm]=2.98 (2H), 4.43 (2H), 7.00 (1H), 7.21-7.36 (2H), 7.56-7.64 (2H), 7.71 (1H), 8.06-8.16 (2H).

LC-MS (Method 1): Rₜ=0.72 min; MS (ESIpos) m/z=295 [M+H]+.

### Intermediate I-8

trans-3-{[3-(1-Benzofuran-2-yl)imidazo[1,2-b]pyridazin-6-yl]oxy}cyclo-butanamine

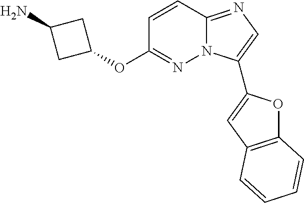 [SMILES: N[C@H]1C[C@H](Oc2ccc3ncc(-c4cc5ccccc5o4)n3n2)C1]

In an ice bath, 1.39 g (7.4 mmol) tert-butyl (trans-3-hydroxycyclobutyl)carbamate were added were slowly added to a suspension of 445 mg (11 mmol) sodium hydride (60% dispersion in mineral oil) in 50 mL of anhydrous THF. After complete addition, stirring at 0° C. was continued for 15 min. 1.0 g (3.7 mmol) of 3-(1-benzofur-2-yl)-6-chloroimidazo[1,2-b]pyridazine were added, the ice bath was removed and the resulting mixture was stirred for 20 h at room temperature.

The reaction mixture was carefully poured into a water. The aqueous layer was extracted with ethyl acetate. The combined organic layers were dried over magnesium sulfate, and concentrated.

The crude product obtained was suspended in 70 mL dichloromethane. 5.7 mL (77 mmol) trifluoro acetic acid were added. The mixture was stirred for 4.5 h.

4.5 mL of ammonia (25% in water) were added. A small amount of DMF was added and the mixture was extracted with a 9:1 mixture of dichloromethane and methanol—The combined organic layers were dried over sodium sulphate and evaporated.

The obtained crude material was digested in methanol to give 920 mg of the title as solid material.

¹H-NMR (300 MHz, DMSO-d₆), δ [ppm]=2.49-2.57 (2H), 3.72 (2H), 5.53 (1H), 7.01 (1H), 7.31 (2H), 7.58-7.67 (2H), 7.71-7.77 (1H), 8.11-8.19 (2H).

LC-MS (Method 3): Rₜ=0.73 min; MS (ESIpos) m/z=321 [M+H]+.

### Intermediate I-9

### 3-(1-Benzofuran-2-yl)-6-(morpholin-2-ylmethoxy)imidazo[1,2-b]pyridazine

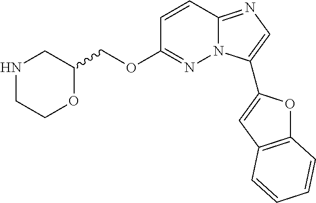 [SMILES: c1ccc2oc(-c3cnc4ccc(OCC5CNCCO5)nn34)cc2c1]

Step 1: In an ice bath, 2.0 g (8.9 mmol) tert.-butyl-2-(hydroxymethyl)morpholine-4-carboxylate were added to 188 mg (7.83 mmol) sodium hydride (60% in mineral oil) in 24 mL anhydrous tetrahydrofurane. After 15 min of stirring in the ice bath, 1.2 g (4.45 mmol) 3-(1-benzofur-2-yl)-6-chloroimidazo[1,2-b]pyridazine were added. The ice bath was removed and the reaction mixture was stirred for 4 days at room temperature.

The reaction mixture was poured into saturated aqueous ammoniumchloride solution, and extracted with ethyl acetate. The combined organic phases were washed with brine, dried over magnesium sulfate, and concentrated. The obtained crude product (3.3 g) was used without further purification in step 2.

Step 2: To 2.2 g of the crude product from step 1 in 36 mL dichloromethane were added 8.9 mL of trifluoroacetic acid. The mixture was stirred for 3 h. Aqueous ammonia was added until the mixture reached basic pH. Brine was added and the mixture extracted with dichloromethane. The organic layer was separated, dried over magnesium sulfate and concentrated. 1.68 g of a solid material were obtained as crude product and used in subsequent steps without further purification.

A small sample (75 mg) was purified by HPLC to give 18 mg of the product as solid material.

¹H-NMR (300 MHz, DMSO-d₆), δ [ppm]=2.64-2.75 (3H), 2.94-3.02 (1H), 3.51 (1H), 3.76-3.92 (1H), 4.45 (2H), 7.06 (1H), 7.23-7.37 (2H), 7.60-7.66 (1H), 7.72 (1H), 8.12-8.19 (2H).

LC-MS (Method 3): Rₜ=0.81 min; MS (ESIpos) m/z=381 [M+H]⁺.

### Intermediate I-10

### 3-(4-Methoxy-1-benzofuran-2-yl)-6-[(2R)-morpholin-2-ylmethoxy]imidazo[1,2-b]pyridazine

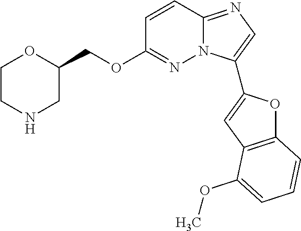 [SMILES: COc1cccc2oc(-c3cnc4ccc(OC[C@H]5CNCCO5)nn34)cc12]

In an ice bath, 191 mg (1.6 mmol) (R)-2-hydroxymethylmorpholine were added to 64 mg (1.6 mmol) sodium hydride (60% in mineral oil) in 24 mL anhydrous tetrahydrofurane. After 15 min of stirring in the ice bath, 120 mg (0.4 mmol) 6-chloro-3-(4-methoxy-1-benzofuran-2-yl)-imidazo[1,2-b]pyridazine were added. The ice bath was removed and the reaction mixture was stirred for 24 h at room temperature.

The reaction mixture was poured into saturated aqueous ammonium chloride solution, and extracted with ethyl acetate. The organic layer was dried over magnesium sulfate, and concentrated. The residue was purified by HPLC to yield 21 mg (14%) product as solid material.

¹H-NMR (300 MHz, DMSO-d₆), δ [ppm]=2.63-2.73 (3H), 2.95 (1H), 3.48 (1H), 3.77 (1H), 3.92 (4H), 4.41 (2H), 6.83 (1H), 7.04 (1H), 7.19-7.33 (2H), 7.53 (1H), 8.02-8.18 (2H).

LC-MS (Method 3): Rₜ=0.81 min; MS (ESIpos) m/z=381 [M+H]⁺.

### Intermediate I-11

### {[3-(1-Benzofuran-2-yl)imidazo[1,2-b]pyridazin-6-yl]oxy}acetic acid

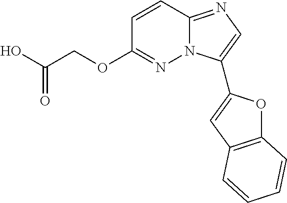 [SMILES: O=C(O)COc1ccc2ncc(-c3cc4ccccc4o3)n2n1]

Step 1: In an ice bath, 945 mg (8.9 mmol) ethyl glycolate were added to 313 mg (7.83 mmol) sodium hydride (60% in mineral oil) in 24 mL anhydrous tetrahydrofurane. After 15 min of stirring in the ice bath, 1.2 g (4.5 mmol) 6-chloro-3-(1-benzofuran-2-yl)-imidazo[1,2-b]pyridazine were added. The ice bath was removed and the reaction mixture was stirred for 16 h at room temperature ° C.

The reaction mixture was poured into saturated aqueous ammonium chloride solution, and extracted with ethyl acetate. The organic layer was washed with brine, dried over magnesium sulfate, and concentrated. The obtained crude product was purified by flash chromatography to give 512 mg of a corresponding ethyl ester.

¹H-NMR (300 MHz, DMSO-d₆), δ [ppm]=1.17 (3H), 4.16 (2H), 5.17 (2H), 7.17 (1H), 7.25-7.37 (2H), 7.46 (1H), 7.60-7.69 (2H), 8.18 (1H), 8.24 (1H).

Step 2: 512 mg of the obtained ethyl ester in 4 mL THF were treated with 38 mg (1.6 mmol) lithium hydroxide in 4 mL water. 500 μL methanol were added and the mixture was stirred at room temperature for 16 h.

The mixture was concentrated under reduced pressure. 100 mL water were added. The mixture was extracted with methyl tert-butyl ether. The aqueous layer was separated, acidified with concentrated aqueous hydrochloric acid and extracted again with methyl tert-butyl ether. The combined organic layers were dried over sodium sulfate and evaporated to give the title compound as a crude product which was used without further purification.

22 mg of the crude product were purified by HPLC to give 7 mg of the title compound as solid material.

¹H-NMR (300 MHz, DMSO-d₆), δ [ppm]=5.05 (2H), 7.13 (1H), 7.23-7.39 (2H), 7.55 (1H), 7.59-7.68 (2H), 8.11-8.29 (2H).

LC-MS (Method 2): Rₜ=1.33 min; MS (ESIpos) m/z=310 [M+H]⁺.

### Intermediate I-12

### (6S)-4-(2,2-dimethylpropanoyl)-6-(hydroxymethyl)piperazin-2-one

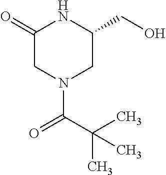

(6S)-4-(2,2-dimethylpropanoyl)-6-(hydroxymethyl)piperazin-2-one was prepared as described in Organic Letters, 2004, Vol. 6, pages 4096-4072.

### Intermediate I-13

### 3-(1-Benzofuran-2-yl)-6-(piperidin-2-ylmethoxy)imidazo[1,2-b]pyridazine

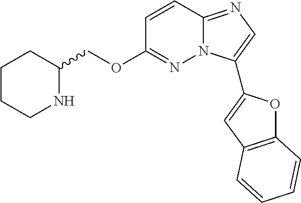 [SMILES: c1ccc2oc(-c3cnc4ccc(OCC5CCCCN5)nn34)cc2c1]

Step 1: In an ice bath, 1.95 g (8.9 mmol) tert.-butyl 2-(hydroxymethyl)piperidine-1-carboxylate were added to 313 mg (7.83 mmol) sodium hydride (60% in mineral oil) in 24 mL anhydrous tetrahydrofurane. After 15 min of stirring in the ice bath, 1.2 g (4.45 mmol) 3-(1-benzofur-2-yl)-6-chloroimidazo[1,2-b]pyridazine were added. The ice bath was removed and the reaction mixture was stirred for 4 days at room temperature.

The reaction mixture was poured into saturated aqueous ammonium chloride solution, and extracted with ethyl acetate. The combined organic phases were washed with brine, dried over magnesium sulfate, and concentrated. The obtained crude product (1.65 g) was used without further purification in step 2.

Step 2: To the crude product from step 1 in 36 mL dichloromethane were added 8.9 mL of trifluoroacetic acid. The mixture was stirred for 3 h. Aqueous ammonia was added until the mixture reached basic pH. Brine was added and the mixture extracted with dichloromethane. The organic layer was separated, dried over magnesium sulfate and concentrated.

The residue was purified by HPLC to give 358 mg (23%) of the product as solid material.

¹H-NMR (500 MHz, DMSO-d₆), δ [ppm]=1.32-1.49 (3H), 1.62 (1H), 1.84 (2H), 2.66-2.71 (1H), 3.09 (1H), 3.17 (1H), 4.40-4.45 (1H), 4.46-4.51 (1H), 7.07 (1H), 7.30-7.35 (1H), 7.36-7.40 (1H), 7.65 (1H), 7.66-7.69 (1H), 7.74-7.78 (1H), 8.19-8.23 (2H).

LC-MS (Method 1): Rₜ=0.82 min; MS (ESIpos) m/z=349 [M+H]⁺

### Intermediate I-14

### 3-(5-Chloro-1-benzofuran-2-yl)-6-{2-[(2S)-pyrrolidin-2-yl]ethoxy}imidazo[1,2-b]pyridazine

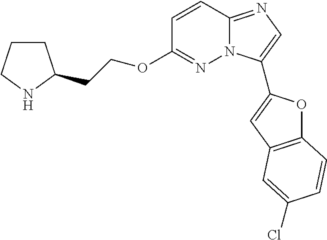 [SMILES: Clc1ccc2oc(-c3cnc4ccc(OCC[C@@H]5CCCN5)nn34)cc2c1]

Step 1: To 9.3 g (40.4 mmol) [(2S)-1-(tert.-butoxycarbonyl)pyrrolidin-2-yl]acetic acid in 116 mL tetrahydrofurane were added dropwise 40 mL of borane-dimethyl sulfide complex. The resulting mixture was stirred for 2 h at 80° C.

The mixture was carefully poured into saturated aqueous sodium hydrogencarbonate solution. The aqueous layer was extracted with methyl-tert.-butylether. The combined organic layers were washed with brine, dried over magnesium sulfate, and concentrated to give 6.2 g of a crude product which was used without further purification in step 2.

Step 2: In an ice bath, 150 mg (0.7 mmol) of the crude product from step 1 were added to 37 mg (0.93 mmol) sodium hydride (60% in mineral oil) in 6 mL anhydrous tetrahydrofurane. After 15 min of stirring in the ice bath, 189 mg (0.47 mmol) 3-(1-benzofur-2-yl)-6-chloroimidazo[1,2-b]pyridazine were added. The ice bath was removed and the reaction mixture was stirred for 18 h at room temperature.

The reaction mixture was poured into water, and extracted with ethyl acetate. The combined organic phases were dried over sodium sulfate, and concentrated. The obtained crude product (327 mg) was used without further purification in step 3.

Step 3: To 327 mg of the crude product from step 2 in 5.8 mL dichloromethane were added 1.3 mL of trifluoroacetic acid. The mixture was stirred for 1.5 h. Aqueous ammonia was added until the mixture reached basic pH. Brine was added and the mixture extracted with dichloromethane. The organic layer was separated, dried over magnesium sulfate and concentrated.

The residue was purified by HPLC to give 45 mg (17%) of the product as solid material.

¹H-NMR (300 MHz, DMSO-d₆), δ [ppm]=1.38-1.53 (1H), 1.67-1.86 (2H), 1.95-2.12 (3H), 2.87-3.06 (2H), 3.31-3.43 (2H), 4.60 (2H), 7.02-7.10 (1H), 7.33-7.41 (1H), 7.67 (2H), 7.79-7.85 (1H), 8.15-8.23 (2H).

LC-MS (Method 3): Rₜ=0.90 min; MS (ESIpos) m/z=383 [M+H]⁺.

### Intermediate I-15

### (6R)-4-(2,2-dimethylpropanoyl)-6-(hydroxymethyl)piperazin-2-one

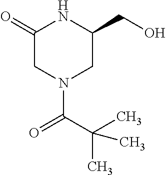

(6R)-4-(2,2-dimethylpropanoyl)-6-(hydroxymethyl)piperazin-2-one was prepared as described in Organic Letters, 2004, Vol. 6, pages 4096-4072.

### Intermediate I-16

### (2R)-2-{[3-(1-Benzofuran-2-yl)imidazo[1,2-b]pyridazin-6-yl]oxy}propan-1-amine

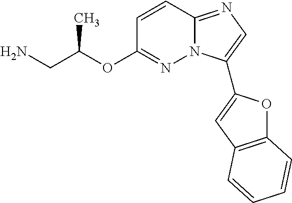 [SMILES: C[C@H](CN)Oc1ccc2ncc(-c3cc4ccccc4o3)n2n1]

In an ice bath, 479 mg (12 mmol) sodium hydride (60% dispersion in mineral oil) were dispensed in 75 mL of anhydrous THF. 600 mg (8 mmol) (2R)-1-aminopropan-2-ol were slowly added. After complete addition, stirring at 0° C. was continued for 15 min. 1.08 g (4 mmol) of 3-(1-benzofur-2-yl)-6-chloroimidazo[1,2-b]-pyridazine were added, the ice bath was removed and the resulting mixture was stirred for 16 h at 40° C.

The reaction mixture was carefully poured into a solution of half-saturated brine. The aqueous layer was extracted with ethyl acetate. The combined organic layers were dried over sodium sulfate, and concentrated.

The crude product was purified by flash chormatography to give 387 mg of the title compound as a solid material.

¹H-NMR (400 MHz, DMSO-d₆), [symbol] [ppm]=1.48 (3H), 3.06-3.23 (2H), 5.44 (1H), 6.95 (1H), 7.22-7.35 (2H), 7.55 (1H), 7.61 (1H), 7.70 (1H), 8.12-8.19 (2H), 8.34 (1H).

LC-MS (Method 3): Rₜ=0.76 min; MS (ESIpos) m/z=309 [M+H]⁺.

### Intermediate II-1

### 3-Bromo-6-chloroimidazo[1,2-b]pyridazine

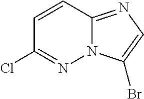 [SMILES: Clc1ccc2ncc(Br)n2n1]

3-Bromo-6-chloroimidazo[1,2-b]pyridazine was synthesised as described for example in WO 2007/147646 or DE 10 2006 029447, e.g. as follows:

### Step 1: Preparation of 6-Chloroimidazo[1,2-b]pyridazine

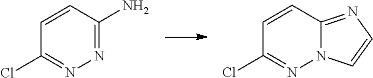 [SMILES: Nc1ccc(Cl)nn1 ; Clc1ccc2nccn2n1]

5.0 g (38.6 mmol) of 3-amino-6-chloropyridazine were heated together with 4.7 mL (40 mmol) of chloroacetaldehyde (55% strength in water) in 15 mL of n-butanol at 120° C. for a period of 5 days. After the reaction was complete, the reaction mixture was added to saturated sodium bicarbonate solution and extracted three times with ethyl acetate. The combined organic phases were then washed with sat. sodium chloride solution and dried over sodium sulfate, and the solvent was removed in vacuo. In the final purification by chromatography on silica gel, 4.17 g (70%) of the desired product were isolated in the form of an amorphous white solid.

¹H-NMR (CHLOROFORM-d): δ [ppm]=7.06 (d, 1H); 7.79 (d, 1H); 7.92, (d, 1H); 7.96 (d, 1H).

### Step 2: Preparation of 3-Bromo-6-chloroimidazo[1,2-b]pyridazine

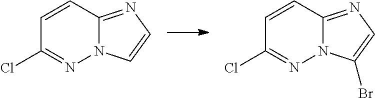 [SMILES: Clc1ccc2nccn2n1 ; Clc1ccc2ncc(Br)n2n1]

478 mg (3.11 mmol) of 6-chloroimidazo[1,2-b]pyridazine were introduced into 10 mL of chloroform under argon and, while cooling in ice, 664 mg (3.73 mmol) of N-bromosuccuinimide were added. After the addition was complete, the reaction mixture was stirred at room temperature overnight. The reaction mixture was then mixed with water and ethyl acetate and, after addition of saturated sodium bicarbonate solution, the phases were separated. The aqueous phase was extracted three more times with ethyl acetate. The combined organic phases were then washed with saturated sodium chloride solution and dried over sodium sulfate. In the final removal of the solvent in vacuo, the desired product was isolated in quantitative yield in the form of an amorphous white solid which was employed without further chromatographic purification in subsequent reactions.

¹H-NMR (CHLOROFORM-d): δ [ppm]=7.12 (d, 1H); 7.79 (s, 1H); 7.90, (d, 1H).

### Intermediate II-2

### 3-Bromo-6-[(2R)-morpholin-2-ylmethoxy]imidazo[1,2-b]pyridazine

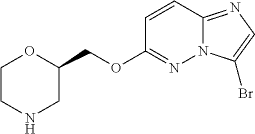 [SMILES: Brc1cnc2ccc(OC[C@H]3CNCCO3)nn12]

At 0−5° C. 1.011 g (6.45 mmol) (2R)-morpholin-2-ylmethanol in 5 mL anhydrous DMF were added to 0.516 g (12.91 mmol) sodium hydride (60% in mineral oil) in 17.4 mL anhydrous DMF. After stirring for 15 minutes on the ice bath 0.75 g (3.23 mmol) 3-bromo-6-chloroimidazo[1,2-b]pyridazine were added. It was stirred 1.5 h at room temperature. The reaction mixture was poured in 180 mL of ice/water. 20 mL saturated aqueous ammonium chloride solution was added. The reaction mixture was stirred 15 min. The in insoluble material was filtered off. The filtrate was extracted three times with 30 mL dichloromethane. The combined organic phases were dried over magnesium sulfate and concentrated. 0.84 g (83%) of the title compound was isolated.

¹H-NMR (300 MHz, DMSO-d₆), δ [ppm]=2.48-2.54 (1H), 2.58-2.66 (2H), 2.82-2.88 (1H), 3.38-3.48 (1H), 3.68-3.79 (2H), 4.25 (2H), 6.95 (1H), 7.71 (1H), 8.02 (1H).

LCMS (Method 1): Rₜ=0.53 min; MS (ESIpos) m/z=313 [M+H]⁺.

### Intermediate II-3

### 4-(1-Benzofuran-4-yl)morpholine

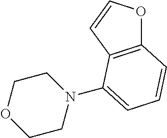 [SMILES: c1cc(N2CCOCC2)c2ccoc2c1]

140 mL anhydrous toluene and 800 mg (1.02 mmol) dichloropalladium-tris(2-methylphenyl)phosphine (1:2) were added to 5 g (25.38 mmol) 4-bromo-1-benzofuran and 4.4 mL (50.50 mmol) morpholine and the solution was purged with argon for 5 min. 3.66 g (38.06 mmol) sodium 2-methylpropan-2-olate were added and the reaction was heated 2 h at 100° C.

It was cooled to room temperature. The reaction was diluted with ethyl acetate and water. The layers were separated, the aqueous phase was extracted two times with ethyl acetate. The combined organic phases were washed three times with water, dried over magnesium sulfate and concentrated. The residue was purified (together with the crude product of a 250 mg 4-bromo-1-benzofuran reaction) on silica gel with a gradient of hexane and ethyl acetate to yield 2.43 g (45%) of the product.

¹H-NMR (300 MHz, DMSO-d₆), δ [ppm]=3.03-3.12 (4H), 3.73-3.80 (4H), 6.65 (1H), 6.98 (1H), 7.12-7.20 (2H), 7.87 (1H).

LCMS (Method 1): Rₜ=1.21 min; MS (ESIpos) m/z=204 [M+H]⁺.

### Intermediate II-4

### [4-(Morpholin-4-yl)-1-benzofuran-2-yl]boronic acid

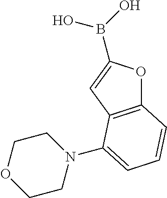 [SMILES: OB(O)c1cc2c(N3CCOCC3)cccc2o1]

To 761 mg (3.7 mmol) of crude 4-(1-benzofuran-4-yl)morpholine in 30 mL anhydrous THF were added 2.2 mL (5.6 mmol) of a solution of n-butyllithium in hexane (c=2.5 M) at −78° C. The mixture was stirred at −78° C. for 1.5 h. 1.3 mL (5.6 mmol) triisopropyl borate were added at −78° C., the cooling bath removed and the mixture was stirred art room temperature for 16 h. Water was added, and the solvent was removed in vacuum. 1.3 g of a crude product was obtained, which was used without further purification.

LCMS (Method 2): Rₜ=0.82 min; MS (ESIpos) m/z=248 [M+H]⁺.

### Intermediate II-5

### tert-Butyl {trans-3-[(3-bromoimidazo[1,2-b]pyridazin-6-yl)oxy]cyclobutyl}-carbamate

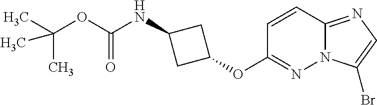 [SMILES: CC(C)(C)OC(=O)N[C@H]1C[C@H](Oc2ccc3ncc(Br)n3n2)C1]

In an ice bath, 2.0 g (10.7 mmol) tert-butyl (trans-3-hydroxycyclobutyl)carbamate were added to 24 mg (10.7 mmol) sodium hydride (60% dispersion in mineral oil) in 124 mL anhydrous THF. After 15 min of stirring on the ice bath, 1.24 g (5.3 mmol) of 3-bromo-6-chloroimidazo[1,2-b]pyridazine were added. The ice bath was removed and the mixture was stirred for 20 h at 40° C.

Water was added. Insoluble precipitate was filtered off and the remaining solution was concentrated. Ethyl acetate and water were added and the mixture was extracted with ethyl acetate. The organic phase was dried over sodium sulfate and the solvent was removed in vacuum. The obtained crude product was used without further purification.

¹H-NMR (500 MHz, DMSO-d₆), δ [ppm]=1.36-1.39 (9H), 2.18-2.28 (4H), 2.45-2.49 (4H), 4.84-4.96 (1H), 5.23-5.32 (1H), 6.96 (1H), 7.74 (1H), 8.05 (1H).

### Intermediate II-6

### 3-Bromo-6-[3-(methylsulfonyl)propoxy]imidazo[1,2-b]pyridazine

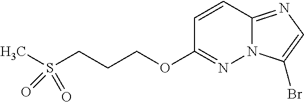 [SMILES: CS(=O)(=O)CCCOc1ccc2ncc(Br)n2n1]

In an ice bath 688 mg (17.2 mmol) sodium hydride (60% dispersion in mineral oil) were dispensed in 72 mL of anhydrous tetrahydrofurane. 1.82 g (17.2 mmol) 3-(methylsulfonyl)propan-1-ol were slowly added. After complete addition, stirring at 0° C. was continued for 15 min. 2.0 g (8.60 mmol) of 3-bromo-6-chloro-imidazo-[1,2-b]pyridazine were added, the ice bath removed and the resulting mixture was stirred for 72 h at room temperature and 24 h at 80° C.

The reaction mixture was carefully poured into saturated ammonium chloride solution. The aqueous layer was extracted with ethyl acetate. The title compound precipitated during the extraction and was filterred off to give 1.4 g of the title compound as solid material which was used in the subsequent steps without further purification.

LC-MS (Method 1): Rₜ=0.77 min; MS (ESIpos) m/z=335 [M+H]⁺.

### Intermediate II-7

### (2R)-1-(1-Benzofuran-4-yl)-2-(methoxymethyl)pyrrolidine

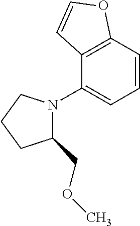 [SMILES: COC[C@H]1CCCN1c1cccc2occc12]

140 mL anhydrous toluene and 800 mg (1.02 mmol) dichloropalladium-tris(2-methylphenyl)phosphine (1:2) were added to 5 g (25.38 mmol) 4-bromo-1-benzofuran and 8.8 mL (71.31 mmol) (2R)-2-(methoxymethyl)pyrrolidine and the solution was purged with argon for 5 min. 3.66 g (38.06 mmol) sodium 2-methylpropan-2-olate were added and the reaction was heated 16 h at 100° C.

It was cooled to room temperature. The reaction was diluted with ethyl acetate and water. The layers were separated, the aqueous phase was extracted twice with ethyl acetate. The combined organic phases were washed three times with water, dried over magnesium sulfate and concentrated. The residue was purified (together with the crude product of a 250 mg 4-bromo-1-benzofuran reaction) on silica gel with a gradient of hexane and ethyl acetate to yield 1.19 g (19%) of the product.

¹H-NMR (300 MHz, DMSO-d₆), δ [ppm]=1.83-2.05 (4H), 3.15-3.26 (4H), 3.26-3.35 (1H, and water signal), 3.42 (1H), 3.58-3.66 (1H), 4.05-4.13 (1H), 6.30 (1H), 6.80 (1H), 6.99 (1H), 7.05 (1H), 7.74 (1H).

LCMS (Method 1): Rₜ=1.35 min; MS (ESIpos) m/z=232 [M+H]⁺.

### Intermediate II-8

### {4-[(2R)-2-(Methoxymethyl)pyrrolidin-1-yl]-1-benzofuran-2-yl}boronic acid

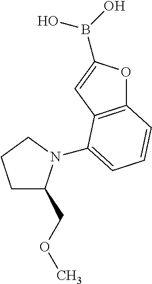 [SMILES: COC[C@H]1CCCN1c1cccc2oc(B(O)O)cc12]

At −78° C. 3.5 mL (8.75 mmol) of a 2.5 M solution of n-butyllithium in hexane were added dropwise to 1.19 g (5.15 mmol) (2R)-1-(1-benzofuran-4-yl)-2-(methoxymethyl)pyrrolidine in 40 mL anhydrous THF. After stirring 1.5 h at −78° C. 2.0 mL (8.74 mmol) triisopropyl borate were added dropwise. The reaction was stirred at room temperature over night. 1 mL water was added and the solution was concentrated to dryness affording 1.98 g of a solid material which was used without further purification.

LCMS (Method 4): Rₜ=0.62 min; MS (ESIpos) m/z=276 [M+H]⁺.

### Intermediate II-9

### (2S)-1-[(3-Bromoimidazo[1,2-b]pyridazin-6-yl)oxy]propan-2-amine

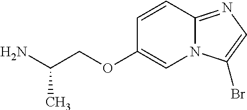

To a stirred suspension of (2S)-2-aminopropan-1-ol (2.91 g) in anhydrous THF (100 mL) and anhydrous DMF (10 mL) was added sodium hydride (60% w/w in oil; 2.07 g) at 0° C. and the mixture was stirred at 0° C. for 30 minutes. 3-Bromo-6-chloroimidazo[1,2-b]pyridazine (6.0 g) was added and the mixture was stirred at room temperature for 16 hours. Water was added and the mixture was extracted with a mixture of dichloromethane and methanol (100:1). The organic phase was dried (sodium sulfate) and the solvent was removed in vacuum. Silicagel chromatography gave a solid that was triturated with a mixture of toluene and cyclohexane to give 4.9 g of the title compound.

¹H-NMR (300 MHz, DMSO-d₆), δ [ppm]=1.05 (3H), 1.63 (2H), 3.10-3.23 (1H), 4.06 (2H), 6.92 (1H), 7.69 (1H), 8.01 (1H).

LCMS (Method 4): Rₜ=0.81 min; MS (ESIpos) m/z=271; 273 [M+H]⁺.

### Intermediate II-10

### (5R)-5-{[(3-Bromoimidazo[1,2-b]pyridazin-6-yl)oxy]methyl}pyrrolidin-2-one

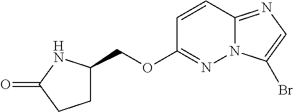 [SMILES: O=C1CC[C@H](COc2ccc3ncc(Br)n3n2)N1]

To a stirred suspension of (5R)-5-(hydroxymethyl)pyrrolidin-2-one (2.23 g) in anhydrous THF (40 mL) and anhydrous DMF (20 mL) was added sodium hydride (60% w/w in oil; 1.03 g) at 0° C. and the mixture was stirred at 0° C. for 30 minutes. 3-Bromo-6-chloroimidazo[1,2-b]pyridazine (3.0 g) was added and the mixture was stirred at room temperature for 60 hours. Water was added and the mixture was extracted with a mixture of dichloromethane and methanol (100:1). The organic phase was dried (sodium sulfate) and the solvent was removed in vacuum. Silicagel chromatography gave a solid that was recrystallized from ethyl acetate to give 2.7 g of the title compound.

¹H-NMR (400 MHz, DMSO-d₆), δ [ppm]=1.81-1.92 (1H), 2.05-2.32 (3H), 3.90-4.01 (1H), 4.18-4.34 (2H), 6.92 (1H), 7.71 (1H), 7.84 (1H), 8.03 (1H).

### Intermediate II-11

### 3-Bromo-6-methoxyimidazo[1,2-b]pyridazine

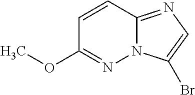

6.0 g (26 mmol) 3-bromo-6-chloro-imidazo[1,2-b]pyridazine were suspended in 225 mL THF. 10 mL (52 mmol) sodium methylate in methanol (c=5.25 mol/L) were added. The mixture was stirred at 75° C. for 24 h.

Water was added and the mixture was extracted with ethyl acetate. The organic layer was washed with brine, dried over sodium sulfate and the solvent was evaporated. The obtained crude product (5.5 g) was used without further purification in the subsequent steps.

¹H-NMR (300 MHz, DMSO-d₆), δ [ppm]=3.92-4.01 (3H), 6.92 (1H), 7.70 (1H), 8.01 (1H).

LCMS (Method 1): Rₜ=0.89 min; MS (ESIpos) m/z=229 [M+H]⁺.

### Intermediate II-12

### 1-(1-Benzofuran-4-yl)-4-phenylpiperazine

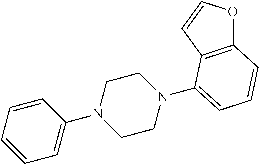 [SMILES: c1ccc(N2CCN(c3cccc4occc34)CC2)cc1]

4.0 g (20 mmol) 4-bromobenzofurane, 4.9 g (30 mmol) 1-phenylpiperazine, 1.2 g (2 mmol) rac-BINAP, 930 mg (1 mmol) Pd₂dba₃ and 5.9 g (61 mmol) sodium 2-methylpropan-2-olate in 112 mL of anhydrous DMF were stirred at 100° C. for 24 h.

The mixture was concentrated under reduced pressure. Water was added and the mixture was extracted with ethyl acetate. The organic layer was dried over sodium sulfate and evaporated. The obtained material was purified by flash chromatography to give 4.6 g of a crude product (approximately 75% purity by LCMS) which was used without further purification.

LCMS (Method 2): Rₜ=1.46 min; MS (ESIpos) m/z=279 [M+H]⁺.

### Intermediate II-13

### [4-(4-Phenylpiperazin-1-yl)-1-benzofuran-2-yl]boronic acid

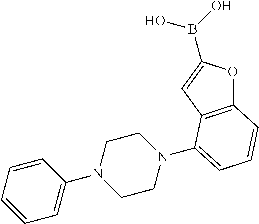 [SMILES: OB(O)c1cc2c(N3CCN(c4ccccc4)CC3)cccc2o1]

To 5.5 g (20 mmol) of 1-(1-benzofuran-4-yl)-4-phenylpiperazine (75% pure) in 202 mL anhydrous THF were added 11.7 mL (29 mmol) of a solution of n-butyllithium in hexane (c=2.5 M) at −78° C. The mixture was stirred at −78° C. for 1.5 h. 6.8 mL (29 mmol) triisopropyl borate were added at −78° C., the cooling bath removed and the mixture was stirred at room temperature for 20 h. Water was added, and the solution was concentrated under reduced pressure. The precipitate was filtered off and washed with water to give 7.6 g of the title compound as a crude product, which was used without further purification in the subsequent steps.

LCMS (Method 2): Rₜ=0.71 min; MS (ESIpos) m/z=324 [M+H]⁺.

### Intermediate II-14

### tert-Butyl 4-(1-benzofuran-4-yl)piperazine-1-carboxylate

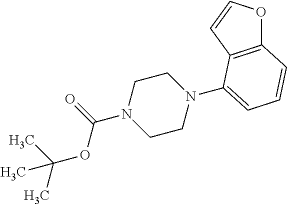 [SMILES: CC(C)(C)OC(=O)N1CCN(c2cccc3occc23)CC1]

5.0 g (25 mmol) 4-bromobenzofurane, 7.1 g (38 mmol) tert-butyl piperazine-1-carboxylate, 1.6 g (2.5 mmol) rac-BINAP, 1.2 g (1.3 mmol) Pd₂dba₃ and 7.3 g (76 mmol) sodium 2-methylpropan-2-olate in 140 mL of anhydrous DMF were stirred at 100° C. for 19 h.

The mixture was concentrated under reduced pressure. Water was added and the mixture was extracted with ethyl acetate. The organic layer was washed with brine, dried over sodium sulfate and evaporated. The obtained material was purified by flash chromatography to give 5.7 g of a crude product (approximately 61% purity by LCMS) which was used without further purification.

LCMS (Method 2): Rₜ=1.42 min; MS (ESIpos) m/z=303 [M+H]⁺.

### Intermediate II-15

### {4-[4-(tert-Butoxycarbonyl)piperazin-1-yl]-1-benzofuran-2-yl}boronic acid

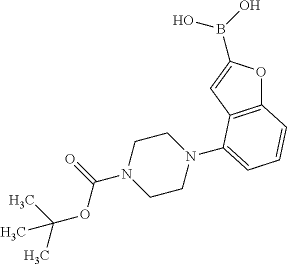 [SMILES: CC(C)(C)OC(=O)N1CCN(c2cccc3oc(B(O)O)cc23)CC1]

To 5.7 g (19 mmol) of tert-butyl 4-(1-benzofuran-4-yl)piperazine-1-carboxylate (61% pure) in 193 mL anhydrous THF were added 11.2 mL (28 mmol) of a solution of n-butyllithium in hexane (c=2.5 M) at −78° C. The mixture was stirred at −78° C. for 1.5 h. 6.5 mL (28 mmol) triisopropyl borate were added at −78° C., the cooling bath removed and the mixture was stirred at room temperature for 21 h. Water was added, and the solution was concentrated under reduced pressure. The precipitate was filtered off and washed with water to give 8.3 g of the title compound as a crude product (approximately 30% pure), which was used without further purification in the subsequent steps.

LCMS (Method 2): Rₜ=1.16 min; MS (ESIpos) m/z=347 [M+H]⁺.

### Intermediate II-16

### 3-Bromo-6-[3-(methylsulfanyl)propoxy]imidazo[1,2-b]pyridazine

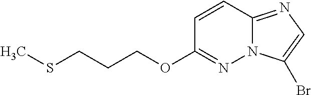 [SMILES: CSCCCOc1ccc2ncc(Br)n2n1]

In an ice bath, 9.1 mL (86 mmol) 3-(methylsulfanyl)propan-1-ol were added to 3.4 g (86 mmol) sodium hydride (60% dispersion in mineral oil) in 200 mL anhydrous THF. After 15 min of stirring on the ice bath, 5.0 g (22 mmol) of 3-bromo-6-chloro-imidazo[1,2-b]pyridazine were added. The ice bath was removed and the mixture was stirred for 24 h at room temperature.

The mixture was poured into saturated aqueous ammonium chloride solution and extracted with ethyl acetate. The organic layer was washed with brine, dried over sodium sulfate and the solvent was removed in vacuum. The crude material was digested with hexane to give 3.2 g of the title compound which was used without further purification.

LCMS (Method 2): Rₜ=1.18 min; MS (ESIpos) m/z=304 [M+H]⁺.

### Intermediate II-17

### [{3-[(3-bromoimidazo[1,2-b]pyridazin-6-yl)oxy]propyl}(methyl)-λ⁴-sulfanylidene]cyanamide

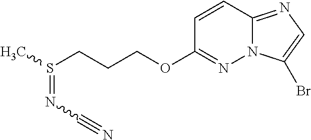 [SMILES: CS(CCCOc1ccc2ncc(Br)n2n1)=NC#N]

To 3.2 g (10.5 mmol) 3-bromo-6-[3-(methylsulfanyl)propoxy]imidazo[1,2-b]-pyridazine, 0.56 g (13.2 mmol) cyanamide in 16 mL methanol were added slowly in portions 1.54 g (13.7 mmol) potassium 2-methylpropan-2-olate. During addition of potassium 2-methylpropan-2-olate the temperature was kept between 20° C. and 25° C. 2.44 g (13.7 mmol) 1-bromopyrrolidine-2,5-dione was added in portions and the resulting mixture was stirred for 1 h at room temperature.

47 mL dichloromethane ware added, followed by 12 ml of an aqueous solution of sodium thiosulfate (10%) and 4 mL of water. The mixture was stirred for 30 min.

The organic layer was extracted with dichloromethane. The organic layer was extracted with brine, dried over sodium sulfate and evaporated. To give 3.9 g of a crude product which was used without further purification in the subsequent step.

LCMS (Method 2): Rₜ=0.72 min; MS (ESIpos) m/z=344 [M+H]⁺.

### Intermediate II-18

### [{3-[(3-Bromoimidazo[1,2-b]pyridazin-6-yl)oxy]propyl}(methyl)oxido-λ⁶-sulfanylidene]cyanamide

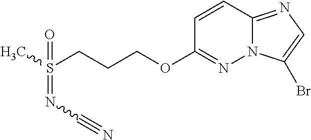 [SMILES: CS(=O)(CCCOc1ccc2ncc(Br)n2n1)=NC#N]

A solution of 9.5 g (68 mmol) potassium carbonate in 50 mL water was added carefully to 14 g (23 mmol) potassium peroxomonosulfate in 110 mL of water. The obtained solution was added over 30 min to 3.9 g (11.4 mmol) of [{3-[(3-bromoimidazo[1,2-b]pyridazin-6-yl)oxy]propyl}-(methyl)-λ⁴-sulfanylidene]-cyanamide in 75 mL dichloromethane, 100 mL methanol and 50 mL ethanol.

After 24 h, a freshly prepared solution of 9.5 g (68 mmol) potassium carbonate in 50 mL water and 14 g (23 mmol) potassium peroxomonosulfate in 110 mL of water was added to the mixture.

After another 24 h, 1.95 g (1.2 mmol) potassium peroxomonosulfate in 10 mL of water were added. 20 mL of methanol were added.

150 mL dichloromethane and 40 mL of an aqueous sodium hydrogen sulfate solution (approximately 40%) were added. The mixture was stirred for 10 min. The aqueous layer was extracted with dichloromethane. The combined organic layers were washed with brine, dried over sodium sulfate and evaporated to yield 3.4 g of a crude product which was used without further purification in the subsequent steps.

¹H-NMR (300 MHz, DMSO-d₆), δ [ppm]=2.14-2.40 (2H), 3.21-3.35 (3H), 3.73-3.88 (2H), 4.39-4.52 (2H), 6.93 (1H), 7.66-7.77 (1H), 7.99-8.11 (1H).

LCMS (Method 2): Rₜ=0.79 min; MS (ESIpos) m/z=358 [M+H]⁺.

### Intermediate II-19

### (2R)-1-[(3-Bromoimidazo[1,2-b]pyridazin-6-yl)oxy]propan-2-amine

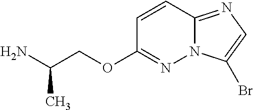

(2R)-1-[(3-Bromoimidazo[1,2-b]pyridazin-6-yl)oxy]propan-2-amine was prepared in analogy to its enantiomer (2S)-1-[(3-bromoimidazo[1,2-b]pyridazin-6-yl)oxy]propan-2-amine.

¹H-NMR (300 MHz, DMSO-d₆), δ [ppm]=1.05 (3H), 3.17 (1H), 4.06 (2H), 6.92 (1H), 7.69 (1H), 8.01 (1H).

LCMS (Method 3): Rₜ=0.55 min; MS (ESIpos) m/z=271; 273 [M+H]⁺.

### Intermediate II-20

### N-Ethyl-N-(2-methoxyethyl)-1-benzofuran-4-amine

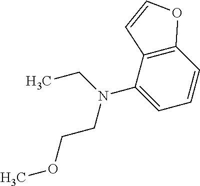 [SMILES: CCN(CCOC)c1cccc2occc12]

To a stirred solution of N-ethyl-2-methoxyethanamine (2.05 g) in toluene (56 mL) was added 4-bromo-1-benzofuran (2.0 g), chloro(2-dicyclohexylphosphino-2′,4′,6′-tri-i-propyl-1,1′-biphenyl)[2-(2-aminoethyl)phenyl]palladium(II) methyl-tert-butylether adduct (822 mg) and X-Phos (474 mg) and the flask was twice degased and backfilled with argon. The mixture was stirred for 5 minutes at room temperature. Sodium 2-methylpropan-2-olate (2.87 g) was added and the flask was twice degased and backfilled with argon. The mixture was heated to reflux for 2 h. A half-saturated solution of sodium bicarbonate was added and the mixture was extracted with ethyl acetate. The organic phase was washed with aqueous ammonium chloride solution and with saturated sodium chloride solution, dried (sodium sulfate) and the solvent was removed in vacuum. Silicagel chromatography gave a solid that was recrystallized from ethyl acetate to give 1.17 g of the title compound.

¹H-NMR (300 MHz, DMSO-d₆), δ [ppm]=1.06 (3H), 3.22 (3H), 3.40 (2H), 3.46-3.50 (4H), 6.51 (1H), 6.88-6.94 (2H), 7.03-7.11 (1H), 7.79 (1H).

LCMS (Method 4): Rₜ=1.28 min; MS (ESIpos) m/z=220 [M+H]⁺.

### Intermediate II-21

### {4-[Ethyl(2-methoxyethyl)amino]-1-benzofuran-2-yl}boronic acid

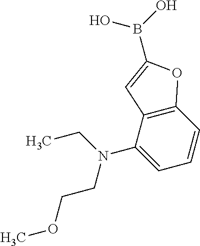 [SMILES: CCN(CCOC)c1cccc2oc(B(O)O)cc12]

To a stirred solution of N-ethyl-N-(2-methoxyethyl)-1-benzofuran-4-amine (1.1 g) in THF (20 mL) was added a solution of n-butyllithium in hexane (3.0 mL; c=2.5 M) at −78° C. The solution was stirred at −78° C. for 1.5 h. Triisopropyl borate (1.51 g) was added at −78° C., and the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 1.93 g of the title compound as a crude product (calculated purity 68%) which was used without purification.

### Intermediate II-22

### 1-(1-Benzofuran-4-yl)-4-methylpiperazine

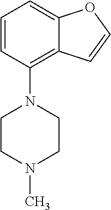 [SMILES: CN1CCN(c2cccc3occc23)CC1]

140 mL anhydrous toluene and 800 mg (1.02 mmol) dichloropalladium-tris(2-methylphenyl)phosphine (1:2) were added to 5 g (25.38 mmol) 4-bromo-1-benzofuran and 11.2 mL (100.97 mmol) 1-methylpiperazine and the solution was purged with argon for 5 min. 3.66 g (38.06 mmol) sodium 2-methylpropan-2-olate were added and the reaction was heated 3 h at 100° C.

It was cooled to room temperature. The reaction was diluted with ethyl acetate and water. The layers were separated, the aqueous phase was extracted twice with ethyl acetate. The combined organic phases were washed three times with water, dried over magnesium sulfate and concentrated. The residue was purified (together with the crude product of a 250 mg 4-bromo-1-benzofuran reaction) on silica gel with a gradient of hexane and ethyl acetate to yield 2.1 g (27%) of the product.

¹H-NMR (300 MHz, DMSO-d₆), δ [ppm]=2.21 (3H), 2.47-2.52 (4H; beginning of the DMSO signal), 3.05-3.14 (4H), 6.60-6.68 (1H), 6.91 (1H), 7.09-7.18 (2H), 7.86 (1H).

LCMS (Method 4): Rₜ=1.06 min; MS (ESIpos) m/z=217 [M+H]⁺.

### Intermediate II-23

### [4-(4-Methylpiperazin-1-yl)-1-benzofuran-2-yl]boronic acid

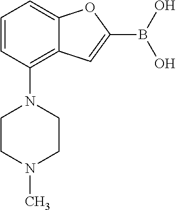 [SMILES: CN1CCN(c2cccc3oc(B(O)O)cc23)CC1]

At −78° C. 6.60 mL (16.51 mmol) of a 2.5 M solution of n-butyllithium in hexane were added dropwise to 2.1 g (9.71 mmol) 1-(1-benzofuran-4-yl)-4-methylpiperazine in 75 mL anhydrous THF. After stirring 1.5 h at −78° C. 3.80 mL (16.51 mmol) triisopropyl borate were added dropwise. The reaction was stirred at rt over night. 1 mL water was added and the solution was concentrated to dryness affording 4.91 g of a solid material which was used without further purification.

LCMS (Method 1): Rₜ=0.55 min; MS (ESIpos) m/z=261 [M+H]⁺.

### Intermediate II-24

### 3-Bromo-6-[(3R)-pyrrolidin-3-yloxy]imidazo[1,2-b]pyridazine

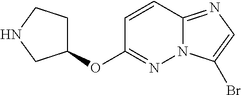 [SMILES: Brc1cnc2ccc(O[C@@H]3CCNC3)nn12]

At 0-5° C. 3.748 g (43.02 mmol) (3R)-pyrrolidin-3-ol were added to 1.72 g (43.02 mmol) sodium hydride (60% in mineral oil) in 116 mL anhydrous DMF. After 15 min on the ice bath 5 g (21.51 mmol) 3-bromo-6-chloroimidazo[1,2-b]pyridazine were added. It was stirred 1.5 h at rt. 0.5 g (6.97 mmol) sodium hydride (60% in mineral oil) were added. It was stirred 0.5 h at rt.

The reaction mixture was concentrated on the rotary evaporator. 250 mL water and 10 mL saturated aqueous ammonium chloride solution were added. It was extracted five times with chloroform. The combined organic phases were washed with water, dried over magnesium sulfate and concentrated. The residue was purified on silica gel using a gradient of dichloromethane and methanol with the addition of 0.01% of aqueous ammonia (32%). 2.09 g (34%) of the title compound was isolated.

¹H-NMR (300 MHz, DMSO-d₆), δ [ppm]=1.82-1.93 (1H), 2.02-2.18 (1H), 2.78-2.99 (3H), 3.19 (1H), 5.30-5.37 (1H), 6.87 (1H), 7.70 (1H), 8.00 (1H).

LCMS (Method 1): Rₜ=0.52 min; MS (ESIpos) m/z=283 [M+H]⁺.

### Intermediate III-01

### 3-Bromo-6-chloroimidazo[1,2-b]pyridazine

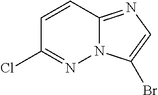 [SMILES: Clc1ccc2ncc(Br)n2n1]

3-Bromo-6-chloroimidazo[1,2-b]pyridazine was synthesised as described for example in WO 2007/147646 or DE 10 2006 029447, e.g. as follows:

### Step 1: Preparation of 6-Chloroimidazo[1,2-b]pyridazine

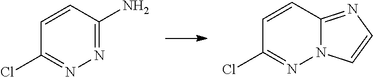 [SMILES: Nc1ccc(Cl)nn1 ; Clc1ccc2nccn2n1]

5.0 g (38.6 mmol) of 3-amino-6-chloropyridazine were heated together with 4.7 mL (40 mmol) of chloroacetaldehyde (55% strength in water) in 15 mL of n-butanol at 120° C. for a period of 5 days. After the reaction was complete, the reaction mixture was added to saturated sodium bicarbonate solution and extracted three times with ethyl acetate. The combined organic phases were then washed with sat. sodium chloride solution and dried over sodium sulfate, and the solvent was removed in vacuo. In the final purification by chromatography on silica gel, 4.17 g (70%) of the desired product were isolated in the form of an amorphous white solid.

¹H-NMR (CHLOROFORM-d): δ [ppm]=7.06 (d, 1H); 7.79 (d, 1H); 7.92, (d, 1H); 7.96 (d, 1H).

### Step 2: Preparation of 3-Bromo-6-chloroimidazo[1,2-b]pyridazine

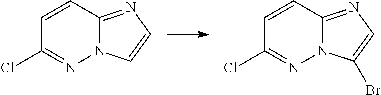 [SMILES: Clc1ccc2nccn2n1 ; Clc1ccc2ncc(Br)n2n1]

478 mg (3.11 mmol) of 6-chloroimidazo[1,2-b]pyridazine were introduced into 10 mL of chloroform under argon and, while cooling in ice, 664 mg (3.73 mmol) of N-bromosuccuinimide were added. After the addition was complete, the reaction mixture was stirred at room temperature overnight. The reaction mixture was then mixed with water and ethyl acetate and, after addition of saturated sodium bicarbonate solution, the phases were separated. The aqueous phase was extracted three more times with ethyl acetate. The combined organic phases were then washed with saturated sodium chloride solution and dried over sodium sulfate. In the final removal of the solvent in vacuo, the desired product was isolated in quantitative yield in the form of an amorphous white solid which was employed without further chromatographic purification in subsequent reactions.

¹H-NMR (CHLOROFORM-d): δ [ppm]=7.12 (d, 1H); 7.79 (s, 1H); 7.90, (d, 1H).

### Intermediate III-02

### (2S)-1-[(3-Bromoimidazo[1,2-b]pyridazin-6-yl)oxy]propan-2-amine

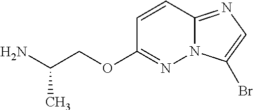

To a stirred suspension of (2S)-2-aminopropan-1-ol (2.91 g) in anhydrous THF (100 mL) and anhydrous DMF (10 mL) was added sodium hydride (60% w/w in oil; 2.07 g) at 0° C. and the mixture was stirred at 0° C. for 30 minutes. 3-Bromo-6-chloroimidazo[1,2-b]pyridazine (6.0 g) was added and the mixture was stirred at room temperature for 16 hours. Water was added and the mixture was extracted with a mixture of dichloromethane and methanol (100:1). The organic phase was dried (sodium sulfate) and the solvent was removed in vacuum. Silicagel chromatography gave a solid that was triturated with a mixture of toluene and cyclohexane to give 4.9 g of the title compound.

¹H-NMR (300 MHz, DMSO-d₆), δ [ppm]=1.05 (3H), 1.63 (2H), 3.10-3.23 (1H), 4.06 (2H), 6.92 (1H), 7.69 (1H), 8.01 (1H).

LCMS (Method 5): Rₜ=0.81 min; MS (ESIpos) m/z=271; 273 [M+H]⁺.

### Intermediate III-03

### (2R)-1-[(3-Bromoimidazo[1,2-b]pyridazin-6-yl)oxy]propan-2-amine

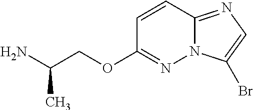

(2R)-1-[(3-Bromoimidazo[1,2-b]pyridazin-6-yl)oxy]propan-2-amine was prepared in analogy to its enantiomer (2S)-1-[(3-bromoimidazo[1,2-b]pyridazin-6-yl)oxy]propan-2-amine.

¹H-NMR (300 MHz, DMSO-d₆), δ [ppm]=1.05 (3H), 3.17 (1H), 4.06 (2H), 6.92 (1H), 7.69 (1H), 8.01 (1H).

LCMS (Method 4): Rₜ=0.55 min; MS (ESIpos) m/z=271; 273 [M+H]⁺.

### Intermediate III-04

### (2R)-2-[(3-Bromoimidazo[1,2-b]pyridazin-6-yl)oxy]propan-1-amine

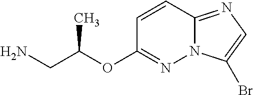

To a stirred suspension of (2R)-1-aminopropan-2-ol (1.78 g) in anhydrous THF (150 mL) and anhydrous NMP (50 mL) was added sodium hydride (60% w/w in oil; 1.72 g) at 0° C. and the mixture was stirred at 0° C. for 15 minutes. 3-Bromo-6-chloroimidazo[1,2-b]pyridazine (5.0 g) was added and the mixture was stirred at room temperature for 72 hours. Water was added and the mixture was extracted with ethyl acetate. The organic phase was washed with saturated sodium chloride solution, dried (sodium sulfate) and the solvent was removed in vacuum. Aminophase silicagel chromatography gave 2.38 g of the title compound.

LCMS (Method 5): Rₜ=0.80 min; MS (ESIpos) m/z=271; 273 [M+H]⁺.

### Intermediate III-05

### 3-Bromo-6-[(3S)-morpholin-3-ylmethoxy]imidazo[1,2-b]pyridazine

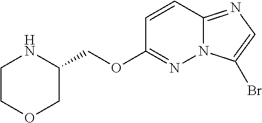 [SMILES: Brc1cnc2ccc(OC[C@@H]3COCCN3)nn12]

To a stirred suspension of (3R)-morpholin-3-ylmethanol hydrochloride (1.0 g) in anhydrous THF (13 mL) and anhydrous DMF (6.5 mL) was added sodium hydride (60% w/w in oil; 0.52 g) at 0° C. and the mixture was stirred at 0° C. for 15 minutes. 3-Bromo-6-chloroimidazo[1,2-b]pyridazine (1.01 g) was added and the mixture was stirred at room temperature for 16 hours. Water was added and the mixture was extracted with a mixture of ethyl acetate and methanol. The organic phase was washed with saturated ammonium chloride solution, dried (sodium sulfate) and the solvent was removed in vacuum. Aminophase silicagel chromatography gave 0.86 g of the title compound.

¹H-NMR (300 MHz, DMSO-d₆), δ [ppm]=2.57 (1H), 2.65-2.81 (2H), 3.02-3.15 (1H), 3.18-3.27 (1H), 3.36 (1H), 3.58-3.67 (1H), 3.80 (1H), 4.18 (2H), 6.92 (1H), 7.70 (1H), 8.02 (1H).

### Intermediate III-06

### 3-Bromo-6-[(3R)-morpholin-3-ylmethoxy]imidazo[1,2-b]pyridazine

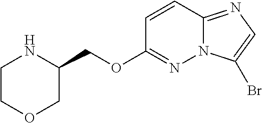 [SMILES: Brc1cnc2ccc(OC[C@H]3COCCN3)nn12]

To a stirred suspension of (3S)-morpholin-3-ylmethanol hydrochloride (0.27 g) in anhydrous THF (10 mL) and anhydrous DMF (10 mL) was added sodium hydride (60% w/w in oil; 0.14 g) at 0° C. and the mixture was stirred at 0° C. for 30 minutes. 3-Bromo-6-chloroimidazo[1,2-b]pyridazine (0.34 g) was added and the mixture was stirred at room temperature for 16 hours. Water was added and the mixture was extracted with a mixture of ethyl acetate and methanol. The organic phase was washed with saturated ammonium chloride solution, dried (sodium sulfate) and the solvent was removed in vacuum. Aminophase silicagel chromatography gave 100 mg of the title compound.

LCMS (Method 2): Rₜ=0.53 min; MS (ESIpos) m/z=313; 315 [M+H]⁺.

### Intermediate III-07

### 3-Bromo-6-[3-(methylsulfonyl)propoxy]imidazo[1,2-b]pyridazine

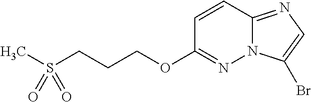 [SMILES: CS(=O)(=O)CCCOc1ccc2ncc(Br)n2n1]

In an ice bath 868 mg (21.7 mmol) sodium hydride (60% dispersion in mineral oil) were dispensed in 150 mL of anhydrous tetrahydrofurane. 3 g (21.7 mmol) 3-(methylsulfonyl)propan-1-ol were slowly added. After complete addition, stirring at 0° C. was continued for 15 min. 2.5 g (10.9 mmol) of 3-bromo-6-chloro-imidazo-[1,2-b]pyridazine were added, the ice bath removed and the resulting mixture was stirred for 24 h at 40° C.

Water was added and the resulting concentration was concentrated. The material was taken up in ethyl acetate, water was added and the precipitate filtered off and washed with water to give 3.3 g of the title compound as solid material which was used in the subsequent steps without further purification.

LC-MS (Method 2): Rₜ=0.76 min; MS (ESIpos) m/z=335 [M+H]⁺.

### Intermediate III-08

### (5R)-5-{[(3-Bromoimidazo[1,2-b]pyridazin-6-yl)oxy]methyl}pyrrolidin-2-one

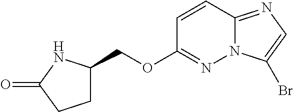 [SMILES: O=C1CC[C@H](COc2ccc3ncc(Br)n3n2)N1]

To a stirred suspension of (5R)-5-(hydroxymethyl)pyrrolidin-2-one (2.23 g) in anhydrous THF (40 mL) and anhydrous DMF (20 mL) was added sodium hydride (60% w/w in oil; 1.03 g) at 0° C. and the mixture was stirred at 0° C. for 30 minutes. 3-bromo-6-chloroimidazo[1,2-b]pyridazine (3.0 g) was added and the mixture was stirred at room temperature for 60 hours. Water was added and the mixture was extracted with a mixture of dichloromethane and methanol (100:1). The organic phase was dried (sodium sulfate) and the solvent was removed in vacuum. Silicagel chromatography gave a solid that was recrystallized from ethyl acetate to give 2.7 g of the title compound.

¹H-NMR (400 MHz, DMSO-d₆), δ [ppm]=1.81-1.92 (1H), 2.05-2.32 (3H), 3.90-4.01 (1H), 4.18-4.34 (2H), 6.92 (1H), 7.71 (1H), 7.84 (1H), 8.03 (1H).

### Intermediate III-09

### (5S)-5-{[(3-Bromoimidazo[1,2-b]pyridazin-6-yl)oxy]methyl}pyrrolidin-2-one

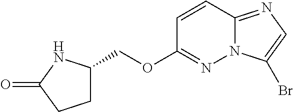 [SMILES: O=C1CC[C@@H](COc2ccc3ncc(Br)n3n2)N1]

In an ice bath 447 mg (11.2 mmol) sodium hydride (60% dispersion in mineral oil) were dispensed in 80 mL of anhydrous tetrahydrofurane. 1.4 g (12 mmol) (5S)-5-(hydroxymethyl)pyrrolidin-2-one were slowly added. After complete addition, stirring at 0° C. was continued for 15 min. 2 g (8.6 mmol) of 3-bromo-6-chloro-imidazo[1,2-b]pyridazine were added, the ice bath removed and the resulting mixture was stirred for 96 h at 60° C. and another 24 h at 80° C.

The reaction mixture was poured into saturated aqueous ammonium chloride solution. The mixture was extracted with ethyl acetate. The organic layer was washed with brine, dried over magnesium sulfate and evaporated to give 2.7 g of the title compound, which was used without further purification.

LC-MS (Method 2): Rₜ=0.72 min; MS (ESIpos) m/z=313 [M+H]⁺.

### Intermediate III-10

### 3-Bromo-6-methoxyimidazo[1,2-b]pyridazine

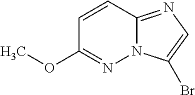

To a suspension of 4 g (17.2 mmol) 3-bromo-6-chloroimidazo[1,2-b]pyridazine in 150 mL anhydrous tetrahydrofurane were added 6.56 mL sodium methanolate in methanol (5.2 mol/L). The mixture was stirred at 75° C. for 17 h.

The reaction mixture was poured into water. The mixture was extracted with ethyl acetate. The organic layer was washed with brine, dried over sodium sulfate and evaporated to give 3.7 g of a crude product which was used without further purification.

LC-MS (Method 2): Rₜ=0.82 min; MS (ESIpos) m/z=228 [M+H]⁺.

### Intermediate III-11

### tert-Butyl {trans-3-[(3-bromoimidazo[1,2-b]pyridazin-6-yl)oxy]cyclobutyl}-carbamate

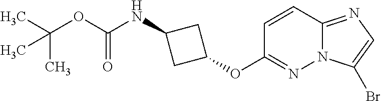 [SMILES: CC(C)(C)OC(=O)N[C@H]1C[C@H](Oc2ccc3ncc(Br)n3n2)C1]

In an ice bath, 2.0 g (10.7 mmol) tert-butyl (trans-3-hydroxycyclobutyl)carbamate were added to 24 mg (10.7 mmol) sodium hydride (60% dispersion in mineral oil) in 124 mL anhydrous THF. After 15 min of stirring on the ice bath, 1.24 g (5.3 mmol) of 3-bromo-6-chloroimidazo[1,2-b]pyridazine were added. The ice bath was removed and the mixture was stirred for 20 h at 40° C.

Water was added. Insoluble precipitate was filtered off and the remaining solution was concentrated. Ethyl acetate and water were added and the mixture was extracted with ethyl acetate. The organic phase was dried over sodium sulfate and the solvent was removed in vacuum. The obtained crude product was used without further purification.

¹H-NMR (500 MHz, DMSO-d₆), δ [ppm]=1.36-1.39 (9H), 2.18-2.28 (4H), 2.45-2.49 (4H), 4.84-4.96 (1H), 5.23-5.32 (1H), 6.96 (1H), 7.74 (1H), 8.05 (1H).

### Intermediate III-12

### (1S,2S)-2-[(3-Bromoimidazo[1,2-b]pyridazin-6-yl)oxy]-2,3-dihydro-1H-inden-1-amine

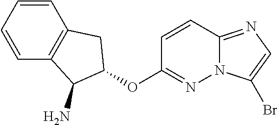 [SMILES: N[C@H]1c2ccccc2C[C@@H]1Oc1ccc2ncc(Br)n2n1]

To a stirred suspension of (1S,2S)-1-aminoindan-2-ol (2.88 g) in anhydrous THF (150 mL) and anhydrous DMF (15 mL) was added sodium hydride (60% w/w in oil; 1.03 g) at 0° C. and the mixture was stirred at 0° C. for 30 minutes. 3-Bromo-6-chloroimidazo[1,2-b]pyridazine (3.0 g) was added and the mixture was stirred at room temperature for 16 hours. Water was added and the mixture was extracted with a mixture of dichloromethane and methanol (100:1). The organic phase was washed with water, dried (sodium sulfate) and the solvent was removed in vacuum. Silicagel chromatography gave 2.55 g of the title compound.

LCMS (Method 2): Rₜ=0.79 min; MS (ESIpos) m/z=345; 347 [M+H]⁺.

### Intermediate III-13

### (1R,2S)-2-[(3-Bromoimidazo[1,2-b]pyridazin-6-yl)oxy]-2,3-dihydro-1H-inden-1-amine

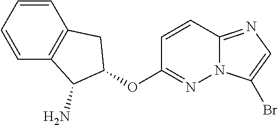 [SMILES: N[C@@H]1c2ccccc2C[C@@H]1Oc1ccc2ncc(Br)n2n1]

To a stirred suspension of (1S,2S)-1-aminoindan-2-ol (1.93 g) in anhydrous THF (100 mL) and anhydrous DMF (10 mL) was added sodium hydride (60% w/w in oil; 0.69 g) at 0° C. and the mixture was stirred at 0° C. for 30 minutes. 3-Bromo-6-chloroimidazo[1,2-b]pyridazine (2.0 g) was added and the mixture was stirred at room temperature for 16 hours. Water was added and the mixture was extracted with a mixture of dichloromethane and methanol (100:1). The organic phase was washed with water, dried (sodium sulfate) and the solvent was removed in vacuum. Silicagel chromatography gave 1.52 g of the title compound.

LCMS (Method 2): Rₜ=0.94 min; MS (ESIpos) m/z=345; 347 [M+H]⁺.

### Intermediate III-14

### N-Methyl-N-[3-(pyrrolidin-1-yl)propyl]furo[3,2-c]pyridin-4-amine

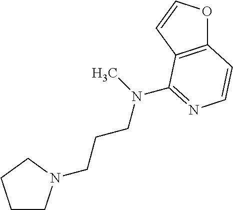 [SMILES: CN(CCCN1CCCC1)c1nccc2occc12]

A mixture of 4-chlorofuro[3,2-c]pyridine (0.9 g), N-methyl-3-(pyrrolidin-1-yl)propan-1-amine (1.0 g) and Hünig base (2.0 mL) was heated to 180° C. in a microwave oven for 4 h. Water was added and the mixture was extracted with ethyl acetate. The organic phase was washed with saturated sodium chloride solution, dried (sodium sulfate) and the solvent was removed in vacuum. Silicagel chromatography gave 823 mg of the title compound.

LCMS (Method 5): Rₜ=1.17 min; MS (ESIpos) m/z=260 [M+H]⁺.

### Intermediate III-15

### (4-{Methyl[3-(pyrrolidin-1-yl)propyl]amino}furo[3,2-c]pyridin-2-yl)boronic acid

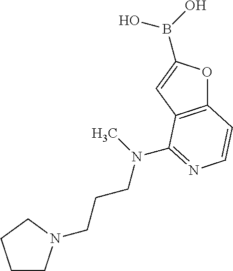 [SMILES: CN(CCCN1CCCC1)c1nccc2oc(B(O)O)cc12]

To a stirred solution of N-methyl-N-[3-(pyrrolidin-1-yl)propyl]furo[3,2-c]pyridin-4-amine (810 mg) in anhydrous THF (10 mL) was added a solution of n-butyllithium in hexane (1.87 mL; c=2.5 M) at −78° C. The solution was stirred at −78° C. for 1.5 h. Triisopropyl borate (0.96 g) was added at −78° C., the mixture was stirred at −78° C. for 0.5 h and allowed to warm up to room temperature within 16 h. Hydrochloric acid (c=2N) was added until pH2 was reached and the reaction mixture was stirred for 30 minutes. The solution was extracted with a mixture of ethyl acetate and hexane (1:1), and an aqueous solution of potassium hydroxide was added to the aqueous phase until pH10 was reached. The solvent was removed in vacuum to give 1.81 g of the title compound as a crude product (calculated purity: 52%), which was used without further purification.

LCMS (Method 5): Rₜ=0.51 min; MS (ESIpos) m/z=304 [M+H]⁺.

### Intermediate III-16

### 3-[Furo[3,2-c]pyridin-4-yl(methyl)amino]propan-1-ol

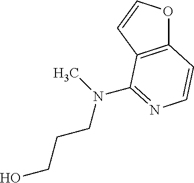

A mixture of 4-chlorofuro[3,2-c]pyridine (1.66 g), and 3-(methylamino)propan-1-ol (4.8 g) was heated to 180° C. in a microwave oven for 2 h. Water was added and the mixture was extracted with ethyl acetate. The organic phase was washed with saturated sodium chloride solution, dried (sodium sulfate) and the solvent was removed in vacuum. Aminophase silicagel chromatography gave 2.1 g of the title compound.

LCMS (Method 5): Rₜ=0.85 min; MS (ESIpos) m/z=207 [M+H]⁺.

### Intermediate III-17

### N-(3-{[tert-Butyl(dimethyl)silyl]oxy}propyl)-N-methylfuro[3,2-c]pyridin-4-amine

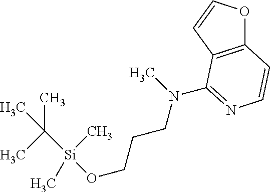 [SMILES: CN(CCCO[Si](C)(C)C(C)(C)C)c1nccc2occc12]

To a stirred solution of 3-[furo[3,2-c]pyridin-4-yl(methyl)amino]propan-1-ol (2.1 g) in THF (100 mL) and DMF (100 mL), triethylamine (4.26 mL), imidazole (1.04 g) and tert-butyl(chloro)dimethylsilane (2.3 g) were added. The mixture was stirred at room temperature for 24 h. A half-saturated solution of sodium bicarbonate was added and the mixture was extracted with ethyl acetate. The organic phase was washed with saturated sodium chloride solution, dried (sodium sulfate) and the solvent was removed in vacuum. Aminophase silicagel chromatography gave 3.4 g of the title compound.

LCMS (Method 3): Rₜ=1.21 min; MS (ESIpos) m/z=321 [M+H]⁺.

### Intermediate III-18

### {4-[(3-{[tert-Butyl(dimethyl)silyl]oxy}propyl)(methyl)amino]furo[3,2-c]pyridin-2-yl}boronic acid

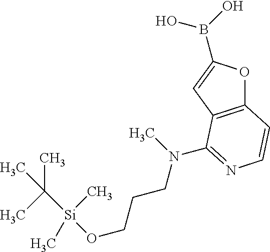 [SMILES: CN(CCCO[Si](C)(C)C(C)(C)C)c1nccc2oc(B(O)O)cc12]

To a stirred solution of N-(3-{[tert-butyl(dimethyl)silyl]oxy}propyl)-N-methylfuro[3,2-c]pyridin-4-amine (3.4 g) in anhydrous THF (30 mL) was added a solution of n-butyllithium in hexane (5.73 mL; c=2.5 M) at −78° C. The solution was stirred at −78° C. for 1.5 h. Triisopropyl borate (2.93 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 3.6 g of the title compound as a crude product, which was used without further purification.

### Intermediate III-19

### N-(2-Methoxyethyl)-N-methylfuro[3,2-c]pyridin-4-amine

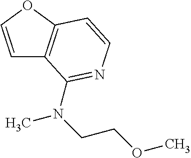

A mixture of 4-chlorofuro[3,2-c]pyridine (3.3 g), 2-methoxy-N-methylethanamine (5.57 g) and Hünig base (7.26 mL) was heated to 180° C. in a microwave oven for 4 h. Water was added and the mixture was extracted with ethyl acetate. The organic phase was washed with saturated sodium chloride solution, dried (sodium sulfate) and the solvent was removed in vacuum. Aminophase silicagel chromatography gave 4.02 g of the title compound.

LCMS (Method 5): Rₜ=0.97 min; MS (ESIpos) m/z=207 [M+H]⁺.

### Intermediate III-20

### {4-[(2-Methoxyethyl)(methyl)amino]furo[3,2-c]pyridin-2-yl}boronic acid

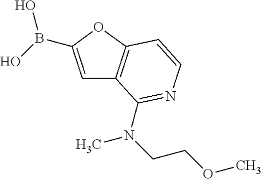 [SMILES: COCCN(C)c1nccc2oc(B(O)O)cc12]

To a stirred solution of N-(2-methoxyethyl)-N-methylfuro[3,2-c]pyridin-4-amine (4.0 g) in anhydrous THF (50 mL) was added a solution of n-butyllithium in hexane (11.6 mL; c=2.5 M) at −78° C. The solution was stirred at −78° C. for 1.5 h. Triisopropyl borate (0.96 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 5.71 g of the title compound as a crude product, which was used without further purification.

LCMS (Method 5): Rₜ=0.38 min; MS (ESIpos) m/z=251 [M+H]⁺.

### Intermediate III-21

### 4-(Pyrrolidin-1-yl)furo[3,2-c]pyridine

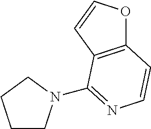 [SMILES: c1cc2occc2c(N2CCCC2)n1]

A mixture of 4-chlorofuro[3,2-c]pyridine (1.25 g) and pyrrolidine (2.8 g) was heated to 180° C. in a microwave oven for 2 h. Water was added and the mixture was extracted with a mixture of dichloromethane and methanol (10:1). The organic phase was washed with water and saturated sodium chloride solution, dried (sodium sulfate) and the solvent was removed in vacuum. Aminophase silicagel chromatography gave 1.18 g of the title compound.

LCMS (Method 2): Rₜ=0.50 min; MS (ESIpos) m/z=189 [M+H]⁺.

### Intermediate III-22

### [4-(Pyrrolidin-1-yl)furo[3,2-c]pyridin-2-yl]boronic acid

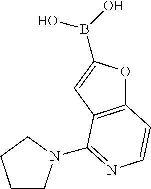 [SMILES: OB(O)c1cc2c(N3CCCC3)nccc2o1]

Starting with 4-(pyrrolidin-1-yl)furo[3,2-c]pyridine (2.35 g), Intermediate 22 was prepared analogously to the procedure for the preparation of Intermediate 20. The title compound was obtained as a crude product, which was used without purification.

### Intermediate III-23

### (3S)-1-(Furo[3,2-c]pyridin-4-yl)-N,N-dimethylpyrrolidin-3-amine

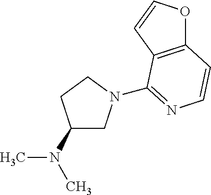 [SMILES: CN(C)[C@H]1CCN(c2nccc3occc23)C1]

A mixture of 4-chlorofuro[3,2-c]pyridine (1.16 g), (3S)—N,N-dimethylpyrrolidin-3-amine (1.0 g) and Hünig base (2.5 mL) was heated to 180° C. in a microwave oven for 8 h. Water was added and the mixture was extracted with a mixture of ethyl acetate and methanol. The organic phase was washed with half-saturated ammonium chloride solution, and with half-saturated sodium bicarbonate solution, dried (sodium sulfate) and the solvent was removed in vacuum. Silicagel chromatography gave 1.1 g of the title compound.

LCMS (Method 2): Rₜ=0.97 min; MS (ESIpos) m/z=232 [M+H]⁺.

### Intermediate III-24

### {4-[(3S)-3-(Dimethylamino)pyrrolidin-1-yl]furo[3,2-c]pyridin-2-yl}boronic acid

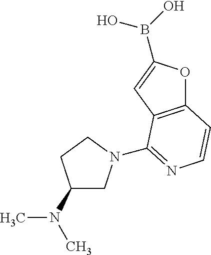 [SMILES: CN(C)[C@H]1CCN(c2nccc3oc(B(O)O)cc23)C1]

To a stirred solution of (3S)-1-(furo[3,2-c]pyridin-4-yl)-N,N-dimethylpyrrolidin-3-amine (1.1 g) in anhydrous THF (12 mL) was added a solution of n-butyllithium in hexane (2.85 mL; c=2.5 M) at −78° C. The solution was stirred at −78° C. for 1.5 h. Triisopropyl borate (1.37 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 1.5 g of the title compound as a crude product (calculated purity: 87%), which was used without further purification.

### Intermediate III-25

### (3R)-1-(Furo[3,2-c]pyridin-4-yl)-N,N-dimethylpyrrolidin-3-amine

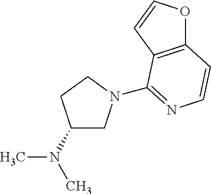 [SMILES: CN(C)[C@@H]1CCN(c2nccc3occc23)C1]

A mixture of 4-chlorofuro[3,2-c]pyridine (1.0 g), (3R)—N,N-dimethylpyrrolidin-3-amine (1.04 g) and Hünig base (2.2 mL) was heated to 180° C. in a microwave oven for 2 h. Water was added and the mixture was extracted with ethyl acetate. The organic phase was washed with half-saturated sodium chloride solution, dried (sodium sulfate) and the solvent was removed in vacuum. Silicagel chromatography gave 1.26 g of the title compound.

LCMS (Method 8): Rₜ=0.92 min; MS (ESIpos) m/z=232 [M+H]⁺.

### Intermediate III-26

### {4-[(3R)-3-(Dimethylamino)pyrrolidin-1-yl]furo[3,2-c]pyridin-2-yl}boronic acid

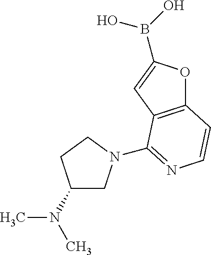 [SMILES: CN(C)[C@@H]1CCN(c2nccc3oc(B(O)O)cc23)C1]

To a stirred solution of (3R)-1-(furo[3,2-c]pyridin-4-yl)-N,N-dimethylpyrrolidin-3-amine (4.7 g) in anhydrous THF (65 mL) was added a solution of n-butyllithium in hexane (12.2 mL; c=2.5 M) at −78° C. The solution was stirred at −78° C. for 1.5 h. Triisopropyl borate (6.2 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 6.57 g of the title compound as a crude product (calculated purity: 85%), which was used without further purification.

### Intermediate III-27

### 4-(Piperidin-1-yl)furo[3,2-c]pyridine

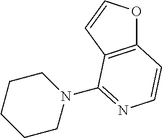 [SMILES: c1cc2occc2c(N2CCCCC2)n1]

A mixture of 4-chlorofuro[3,2-c]pyridine (2.5 g), piperidine (6.72 g) and Hünig base (7.8 mL) was heated to 180° C. in a microwave oven for 2 h. Water was added and the mixture was extracted with ethyl acetate. The organic phase was washed with half-saturated ammonium chloride solution, dried (sodium sulfate) and the solvent was removed in vacuum. Aminophase silicagel chromatography gave 3.1 g of the title compound.

LCMS (Method 3): Rₜ=0.54 min; MS (ESIpos) m/z=203 [M+H]⁺.

### Intermediate III-28

### [4-(Piperidin-1-yl)furo[3,2-c]pyridin-2-yl]boronic acid

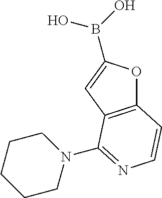 [SMILES: OB(O)c1cc2c(N3CCCCC3)nccc2o1]

To a stirred solution of 4-(piperidin-1-yl)furo[3,2-c]pyridine (3.2 g) in anhydrous THF (40 mL) was added a solution of n-butyllithium in hexane (9.49 mL; c=2.5 M) at −78° C. The solution was stirred at −78° C. for 1.5 h. Triisopropyl borate (4.55 g) was added at −78° C., the mixture was stirred at −78° C. for 0.5 h and allowed to warm up to room temperature within 16 h. Hydrochloric acid was added and the reaction mixture was stirred for 15 minutes. A saturated solution of potassium carbonate was added until pH7 was reached and the solvent was removed in vacuum. The solid residue was stirred with a mixture of chloroform and methanol (10:1) for three times. The combined organic solutions were concentrated in vacuum to give 1.1 g of the title compound as a crude product which was used without further purification.

### Intermediate III-29

### [1-(Furo[3,2-c]pyridin-4-yl)piperidin-4-yl]methanol

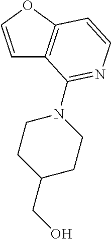 [SMILES: OCC1CCN(c2nccc3occc23)CC1]

A mixture of 4-chlorofuro[3,2-c]pyridine (0.595 g), piperidin-4-ylmethanol (0.53 g) and Hünig base (1.3 mL) was heated to 180° C. in a microwave oven for 2 h. Water was added and the mixture was extracted with a mixture of ethyl acetate and methanol (100:1). The organic phase was washed with half-saturated ammonium chloride solution, dried (sodium sulfate) and the solvent was removed in vacuum. Silicagel chromatography gave 800 mg of the title compound.

LCMS (Method 2): Rₜ=0.50 min; MS (ESIpos) m/z=233 [M+H]⁺.

### Intermediate III-30

### 4-[4-({[tert-Butyl(dimethyl)silyl]oxy}methyl)piperidin-1-yl]furo[3,2-c]pyridine

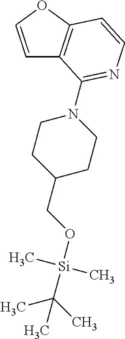 [SMILES: CC(C)(C)[Si](C)(C)OCC1CCN(c2nccc3occc23)CC1]

To a stirred solution of [1-(furo[3,2-c]pyridin-4-yl)piperidin-4-yl]methanol (800 mg) in THF (70 mL), triethylamine (1.44 mL), imidazole (352 mg) and tert-butyl(chloro)dimethylsilane (779 mg) were added. The mixture was stirred at room temperature for 2 h. Further imidazole (352 mg) and tert-butyl(chloro)dimethylsilane (779 mg) were added and the mixture was stirred at room temperature for 4 h. A half-saturated solution of sodium bicarbonate was added and the mixture was extracted with ethyl acetate. The organic phase was washed with half-saturated ammonium chloride solution, dried (sodium sulfate) and the solvent was removed in vacuum. Silicagel chromatography followed by aminophase silicagel chromatography gave 1.0 g of the title compound.

### Intermediate III-31

### {4-[4-({[tert-Butyl(dimethyl)silyl]oxy}methyl)piperidin-1-yl]furo[3,2-c]pyridin-2-yl}boronic acid

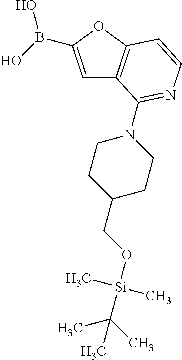 [SMILES: CC(C)(C)[Si](C)(C)OCC1CCN(c2nccc3oc(B(O)O)cc23)CC1]

To a stirred solution of 4-[4-({[tert-butyl(dimethyl)silyl]oxy}methyl)piperidin-1-yl]furo[3,2-c]pyridine (1.0 g) in anhydrous THF (8.0 mL) was added a solution of n-butyllithium in hexane (1.73 mL; c=2.5 M) at −78° C. The solution was stirred at −78° C. for 1.5 h. Triisopropyl borate (0.83 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 1.3 g of the title compound as a crude product (calculated purity: 85%), which was used without further purification.

### Intermediate III-32

### 1-(Furo[3,2-c]pyridin-4-yl)-N,N-dimethylpiperidin-4-amine

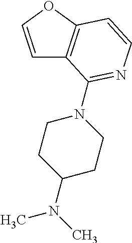 [SMILES: CN(C)C1CCN(c2nccc3occc23)CC1]

A mixture of 4-chlorofuro[3,2-c]pyridine (0.595 g), N,N-dimethylpiperidin-4-amine (0.59 g) and Hünig base (1.3 mL) was heated to 180° C. in a microwave oven for 2 h. Water was added and the mixture was extracted with a mixture of dichloromethane and methanol (10:1). The organic phase was washed with half-saturated ammonium chloride solution, dried (sodium sulfate) and the solvent was removed in vacuum. Aminophase silicagel chromatography gave 500 mg of the title compound.

¹H-NMR (400 MHz, CHLOROFORM-d), δ [ppm]=1.55-1.70 (2H), 1.96 (2H), 2.32 (6H), 2.36-2.47 (1H), 2.93-3.09 (2H), 4.39 (2H), 6.77-6.84 (1H), 6.89-6.95 (1H), 7.52 (1H), 8.03 (1H).

### Intermediate III-33

### {4-[4-(Dimethylamino)piperidin-1-yl]furo[3,2-c]pyridin-2-yl}boronic acid

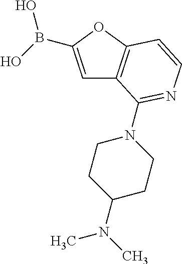 [SMILES: CN(C)C1CCN(c2nccc3oc(B(O)O)cc23)CC1]

To a stirred solution of 1-(furo[3,2-c]pyridin-4-yl)-N,N-dimethylpiperidin-4-amine (500 mg) in anhydrous THF (6 mL) was added a solution of n-butyllithium in hexane (1.22 mL; c=2.5 M) at −78° C. The solution was stirred at −78° C. for 1.5 h. Triisopropyl borate (0.59 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 0.70 g of the title compound as a crude product (calculated purity: 85%), which was used without further purification.

### Intermediate III-34

### N-(2-Methoxyethyl)furo[3,2-c]pyridin-4-amine

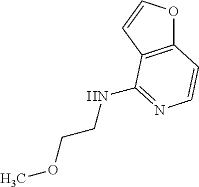

A mixture of 4-chlorofuro[3,2-c]pyridine (2.0 g) and 2-methoxyethanamine (4.89 g) was heated to 180° C. in a microwave oven for 3 h. Water was added and the mixture was extracted with a mixture of ethyl acetate and methanol (100:1). The organic phase was washed with saturated sodium chloride solution, dried (sodium sulfate) and the solvent was removed in vacuum. Silicagel chromatography gave 1.5 g of the title compound.

LCMS (Method 3): Rₜ=0.47 min; MS (ESIpos) m/z=193 [M+H]⁺.

### Intermediate III-35

### N-(Furo[3,2-c]pyridin-4-yl)-N-(2-methoxyethyl)propanamide

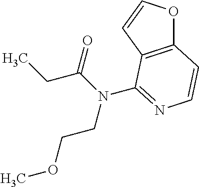 [SMILES: CCC(=O)N(CCOC)c1nccc2occc12]

To a stirred solution of N-(2-methoxyethyl)furo[3,2-c]pyridin-4-amine (1.0 g) in dichloromethane (50 mL) was added Hünig base (1.8 mL) and pyridine (0.08 mL). The mixture was cooled to 0° C., propanoyl chloride (0.79 mL) was added and the mixture was stirred at room temperature for 6 h. A half-saturated solution of ammonium chloride was added and the mixture was extracted with ethyl acetate and methanol (100:1 mixture). The organic phase was washed with saturated sodium chloride solution, dried (sodium sulfate) and the solvent was removed in vacuum. Aminophase silicagel chromatography gave 1.23 g of the title compound.

LCMS (Method 2): Rₜ=0.80 min; MS (ESIpos) m/z=249 [M+H]⁺.

### Intermediate III-36

### N-(2-Methoxyethyl)-N-propylfuro[3,2-c]pyridin-4-amine

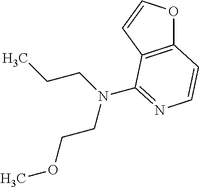 [SMILES: CCCN(CCOC)c1nccc2occc12]

To a stirred solution of N-(furo[3,2-c]pyridin-4-yl)-N-(2-methoxyethyl)propanamide (1.12 g) in tetrahydrofurane (30 mL) was added borane dimethylsulfide complex (0.97 mL) at 0° C. The solution was allowed to warm up to room temperature, and was stirred at room temperature for 16 h. Water was added and the mixture was extracted with ethyl acetate. The organic phase was washed with saturated sodium chloride solution, dried (sodium sulfate) and the solvent was removed in vacuum. Aminophase silicagel chromatography gave 495 mg of the title compound.

LCMS (Method 2): Rₜ=0.61 min; MS (ESIpos) m/z=235 [M+H]⁺.

### Intermediate III-37

### {4-[(2-Methoxyethyl)(propyl)amino]furo[3,2-c]pyridin-2-yl}boronic acid

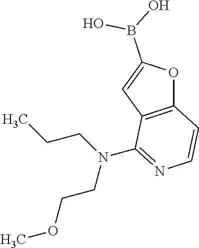 [SMILES: CCCN(CCOC)c1nccc2oc(B(O)O)cc12]

To a stirred solution of N-(2-methoxyethyl)-N-propylfuro[3,2-c]pyridin-4-amine (440 mg) in anhydrous THF (15 mL) was added a solution of n-butyllithium in hexane (1.13 mL; c=2.5 M) at −78° C. The solution was stirred at −78° C. for 1.5 h. Triisopropyl borate (0.54 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added, the solution was extracted with a mixture of ethyl acetate and hexane (1:1) and the aqueous phase was lyophilized to give 400 mg of the title compound as a crude product, which was used without further purification.

### Intermediate III-38

### N-Methyl-N-(1-methylpiperidin-4-yl)furo[3,2-c]pyridin-4-amine

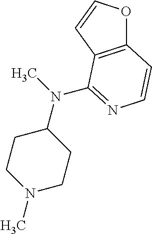 [SMILES: CN1CCC(N(C)c2nccc3occc23)CC1]

A mixture of 4-chlorofuro[3,2-c]pyridine (1.5 g) and N,1-dimethylpiperidin-4-amine (5.0 g) was heated to 190° C. in a microwave oven for 5 h. Water was added and the mixture was extracted with ethyl acetate. The organic phase was washed with saturated sodium chloride solution, dried (sodium sulfate) and the solvent was removed in vacuum. Aminophas silicagel chromatography gave 540 mg of the title compound.

LCMS (Method 5): Rₜ=1.02 min; MS (ESIpos) m/z=246 [M+H]⁺.

### Intermediate III-39

### {4-[Methyl(1-methylpiperidin-4-yl)amino]furo[3,2-c]pyridin-2-yl}boronic acid

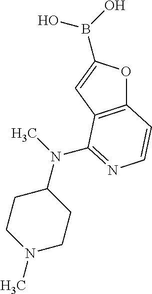 [SMILES: CN1CCC(N(C)c2nccc3oc(B(O)O)cc23)CC1]

To a stirred solution of N-methyl-N-(1-methylpiperidin-4-yl)furo[3,2-c]pyridin-4-amine (535 mg) in anhydrous THF (10 mL) was added a solution of n-butyllithium in hexane (1.30 mL; c=2.5 M) at −78° C. The solution was stirred at −78° C. for 1.5 h. Triisopropyl borate (0.67 g) was added at −78° C., the mixture was stirred at −78° C. for 0.5 h and allowed to warm up to room temperature within 16 h. Hydrochloric acid was added and the reaction mixture was stirred for 15 minutes. The solution was extracted with a mixture of ethyl acetate and hexane (1:1) and a solution of potassium hydroxide was added to the aqueous phase until pH 10 was reached. The aqueous solution was concentrated in vacuum to give 2.4 g of the title compound as a crude product (calculated purity: 26%), which was used without further purification.

### Intermediate III-40

### 4-[(2R,6S)-2,6-Dimethylmorpholin-4-yl]furo[3,2-c]pyridine

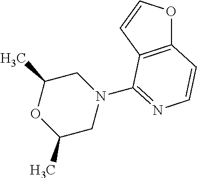 [SMILES: C[C@@H]1CN(c2nccc3occc23)C[C@H](C)O1]

A mixture of 4-chlorofuro[3,2-c]pyridine (3.0 g) and (2R,6S)-2,6-dimethylmorpholine (11.3 g) was heated to 180° C. in a microwave oven for 2 h. Water was added and the mixture was extracted with ethyl acetate. The organic phase was washed with saturated sodium chloride solution, dried (sodium sulfate) and the solvent was removed in vacuum. Aminophase silicagel chromatography gave 4.16 g of the title compound.

LCMS (Method 5): Rₜ=1.09 min; MS (ESIpos) m/z=233 [M+H]⁺.

### Intermediate III-41

### {4-[(2R,6S)-2,6-Dimethylmorpholin-4-yl]furo[3,2-c]pyridin-2-yl}boronic acid

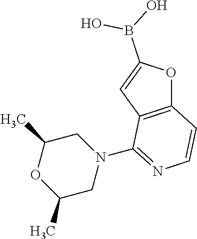 [SMILES: C[C@@H]1CN(c2nccc3oc(B(O)O)cc23)C[C@H](C)O1]

To a stirred solution of 4-[(2R,6S)-2,6-dimethylmorpholin-4-yl]furo[3,2-c]pyridine (4.06 g) in anhydrous THF (45 mL) was added a solution of n-butyllithium in hexane (10.5 mL; c=2.5 M) at −78° C. The solution was stirred at −78° C. for 1.5 h. Triisopropyl borate (5.37 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 6.38 g of the title compound as a crude product (calculated purity: 75%), which was used without further purification.

### Intermediate III-42

### tert-Butyl 4-(furo[3,2-c]pyridin-4-yl)piperazine-1-carboxylate

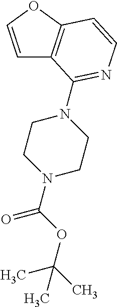 [SMILES: CC(C)(C)OC(=O)N1CCN(c2nccc3occc23)CC1]

A mixture of 4-chlorofuro[3,2-c]pyridine (3.0 g), tert-butyl piperazine-1-carboxylate (5.1 g) and Hünig base (6.6 mL) was heated to 180° C. in a microwave oven for 0.5 h. Water was added and the mixture was extracted with ethyl acetate. The organic phase was washed with saturated sodium chloride solution, dried (sodium sulfate) and the solvent was removed in vacuum. Silicagel chromatography followed by aminophase silicagel chromatography gave 3.8 g of the title compound.

LCMS (Method 2): Rₜ=0.73 min; MS (ESIpos) m/z=304 [M+H]⁺.

### Intermediate III-43

### {4-[4-(tert-Butoxycarbonyl)piperazin-1-yl]furo[3,2-c]pyridin-2-yl}boronic acid

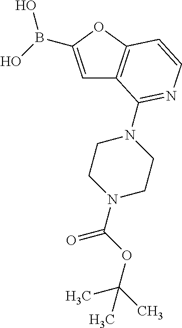 [SMILES: CC(C)(C)OC(=O)N1CCN(c2nccc3oc(B(O)O)cc23)CC1]

To a stirred solution of tert-butyl 4-(furo[3,2-c]pyridin-4-yl)piperazine-1-carboxylate (3.8 g) in anhydrous THF (31 mL) was added a solution of n-butyllithium in hexane (7.52 mL; c=2.5 M) at −78° C. The solution was stirred at −78° C. for 1.5 h. Triisopropyl borate (3.6 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 4.9 g of the title compound as a crude product (calculated purity: 89%), which was used without further purification.

### Intermediate III-44

### 4-(4-Methylpiperazin-1-yl)furo[3,2-c]pyridine

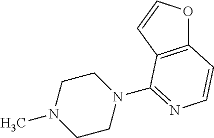 [SMILES: CN1CCN(c2nccc3occc23)CC1]

A mixture of 4-chlorofuro[3,2-c]pyridine (2.25 g) and 1-methylpiperazine (7.1 g) was heated to 180° C. in a microwave oven for 2 h. Water was added and the mixture was extracted with ethyl acetate. The organic phase was washed with half-saturated ammonium chloride solution and saturated sodium chloride solution, dried (sodium sulfate) and the solvent was removed in vacuum. Silicagel chromatography gave 1.46 g of the title compound.

LCMS (Method 2): Rₜ=0.37 min; MS (ESIpos) m/z=218 [M+H]⁺.

### Intermediate III-45

### [4-(4-Methylpiperazin-1-yl)furo[3,2-c]pyridin-2-yl]boronic acid

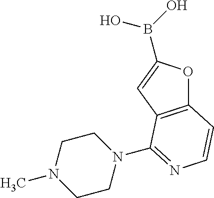 [SMILES: CN1CCN(c2nccc3oc(B(O)O)cc23)CC1]

To a stirred solution of 4-(4-methylpiperazin-1-yl)furo[3,2-c]pyridine (1.4 g) in anhydrous THF (20 mL) was added a solution of n-butyllithium in hexane (3.87 mL; c=2.5 M) at −78° C. The solution was stirred at −78° C. for 1.5 h. Triisopropyl borate (1.85 g) was added at −78° C., the mixture was stirred at −78° C. for 0.5 h and allowed to warm up to room temperature within 16 h. Hydrochloric acid (6 mL, c=2 M) was added, the reaction mixture was stirred for 15 minutes. The solution was extracted with a mixture of ethyl acetate and hexane (2:1) and a solution of potassium carbonate was added to the aqueous phase until pH 7.5 was reached. The aqueous solution was concentrated in vacuum to give 5.85 g of the title compound as a crude product (calculated purity: 29%), which was used without further purification.

### Intermediate III-46

### 6-Chloro-3-[4-(4-methylpiperazin-1-yl)furo[3,2-c]pyridin-2-yl]imidazo[1,2-b]pyridazine

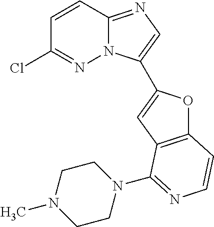 [SMILES: CN1CCN(c2nccc3oc(-c4cnc5ccc(Cl)nn45)cc23)CC1]

To a stirred solution of 3-Bromo-6-chloro-imidazo[1,2-b]pyridazine (1.36 g) in 1-propanol (42 mL) was added potassium carbonate solution (8.8 mL; c=2 M), crude [4-(4-methylpiperazin-1-yl)furo[3,2-c]pyridin-2-yl]boronic acid (29% w/w; 5.68 g), triphenylphosphine (153 mg) and PdCl₂(PPh₃)₂ (420 mg). The mixture was heated to reflux for 3 h. Water was added and the mixture was extracted with a mixture of dichloromethane and methanol (10:1). The organic phase was washed with saturated sodium chloride solution, dried (sodium sulfate) and the solvent was removed in vacuum. The solid residue was triturated with a mixture of ethyl acetate and hexane (1:1) to give 1.35 g of a crude product. Silicagel chromatography gave 1.04 g of the title compound.

LCMS (Method 2): Rₜ=0.64 min; MS (ESIpos) m/z=369 [M+H]⁺.

### Intermediate III-47

### 4-(4-tert-Butylpiperazin-1-yl)furo[3,2-c]pyridine

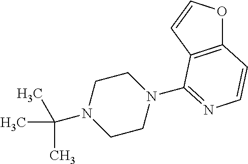 [SMILES: CC(C)(C)N1CCN(c2nccc3occc23)CC1]

A mixture of 4-chlorofuro[3,2-c]pyridine (3.0 g), 1-tert-butylpiperazine (3.77 g) and Hünig base (6.6 mL) was heated to 180° C. in a microwave oven for 2 h. Water was added and the mixture was extracted with ethyl acetate. The organic phase was washed with half-saturated ammonium chloride solution, dried (sodium sulfate) and the solvent was removed in vacuum. Aminophase silicagel chromatography gave 4.6 g of the title compound.

LCMS (Method 2): Rₜ=0.54 min; MS (ESIpos) m/z=260 [M+H]⁺.

### Intermediate III-48

### [4-(4-tert-Butylpiperazin-1-yl)furo[3,2-c]pyridin-2-yl]boronic acid

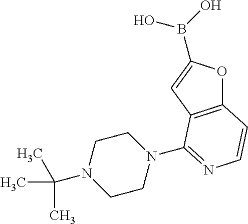 [SMILES: CC(C)(C)N1CCN(c2nccc3oc(B(O)O)cc23)CC1]

To a stirred solution of 4-(4-tert-butylpiperazin-1-yl)furo[3,2-c]pyridine (4.6 g) in anhydrous THF (45 mL) was added a solution of n-butyllithium in hexane (10.6 mL; c=2.5 M) at −78° C. The solution was stirred at −78° C. for 1.5 h. Triisopropyl borate (5.1 g) was added at −78° C., the mixture was stirred at −78° C. for 0.5 h and allowed to warm up to room temperature within 16 h. Hydrochloric acid was added, the reaction mixture was stirred for 15 minutes. Water was added and the solution was extracted with a mixture of ethyl acetate and hexane (2:1) and a solution of potassium hydroxide was added to the aqueous phase until pH 6 was reached. The aqueous solution was concentrated in vacuum to give 9.66 g of the title compound as a crude product (calculated purity: 55%), which was used without further purification.

### Intermediate III-49

### 4-(Morpholin-4-yl)furo[3,2-c]pyridine

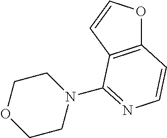 [SMILES: c1cc2occc2c(N2CCOCC2)n1]

A mixture of 4-chlorofuro[3,2-c]pyridine (12.0 g) and morpholine (34 g) was heated to 180° C. in a microwave oven for 2 h. Water was added and the mixture was extracted with ethyl acetate. The organic phase was washed with half-saturated sodium chloride solution, dried (sodium sulfate) and the solvent was removed in vacuum. Aminophase slicagel chromatography gave 13.4 g of the title compound.

LCMS (Method 5): Rₜ=0.88 min; MS (ESIpos) m/z=205 [M+H]⁺.

### Intermediate III-50

### [4-(Morpholin-4-yl)furo[3,2-c]pyridin-2-yl]boronic acid

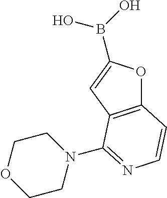 [SMILES: OB(O)c1cc2c(N3CCOCC3)nccc2o1]

To a stirred solution of 4-(morpholin-4-yl)furo[3,2-c]pyridine (15.8 g) in anhydrous THF (190 mL) was added a solution of n-butyllithium in hexane (46.5 mL; c=2.5 M) at −78° C. The solution was stirred at −78° C. for 1.5 h. Triisopropyl borate (23.8 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 24.3 g of the title compound as a crude product (calculated purity: 78%), which was used without further purification.

### Intermediate III-51

### 6-Chloro-3-[4-(morpholin-4-yl)furo[3,2-c]pyridin-2-yl]imidazo[1,2-b]pyridazine

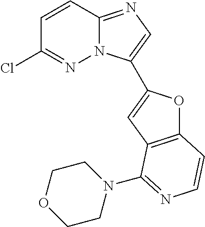 [SMILES: Clc1ccc2ncc(-c3cc4c(N5CCOCC5)nccc4o3)n2n1]

To a stirred solution of 3-Bromo-6-chloro-imidazo[1,2-b]pyridazine (5.52 g) in 1-propanol (170 mL) was added potassium carbonate solution (36 mL; c=2 M), crude [4-(morpholin-4-yl)furo[3,2-c]pyridin-2-yl]boronic acid (72% w/w; 9.0 g), triphenylphosphine (623 mg) and PdCl₂(PPh₃)₂ (1.70 g). The mixture was heated to reflux for 1 h. The warm mixture was filtered through Celite the solvent was removed in vacuum. A half-saturated solution of sodium bicarbonate was added and the mixture was extracted with a mixture of dichloromethane and methanol. The organic phase was washed with saturated sodium chloride solution, dried (sodium sulfate) and the solvent was removed in vacuum. Silicagel chromatography gave 4.28 g of the title compound.

LCMS (Method 5): Rₜ=1.11 min; MS (ESIpos) m/z=356 [M+H]⁺.

### Intermediate III-52

### N-Ethyl-N-(2-methoxyethyl)furo[3,2-c]pyridin-4-amine

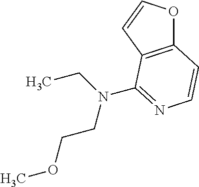 [SMILES: CCN(CCOC)c1nccc2occc12]

3 g (20 mmol) 4-chlorofuro[3,2-c]pyridine, 7 mL (59 mmol) N-(2-methoxyethyl)ethylamine and 3.4 mL (20 mmol) N-ethyl-N-isopropylpropan-2-amine were heated to 180° C. for 6 h in a microwave oven.

The reaction mixture was poured into brine and extracted with ethyl acetate. The organic layer was dried over magnesium sulfate and evaporated. The crude product was purified by flash chromatography to give 3.1 g of the title compound.

LCMS (Method 3): Rₜ=0.57 min; MS (ESIpos) m/z=221 [M+H]⁺.

### Intermediate III-53

### {4-[Ethyl(2-methoxyethyl)amino]furo[3,2-c]pyridin-2-yl}boronic acid

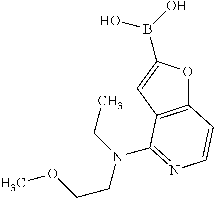 [SMILES: CCN(CCOC)c1nccc2oc(B(O)O)cc12]

3.1 g (6 mmol) N-ethyl-N-(2-methoxyethyl)furo[3,2-c]pyridin-4-amine in 30 mL anhydrous THF was cooled to −78° C. 8.4 mL (21 mmol) of a 2.5 M solution of n-butyl lithium in hexane was added. The mixture was stirred for 90 min at −78° C. 4.9 mL (21 mmol) of triisopropyl borate was added at −78° C. The cooling bath was removed and the mixture was stirred at room temperature for 18 h.

11 mL of 2 M aqueous hydrochloric acid were added. The mixture was concentrated. Toluol was added and evaporated. Acetone was added and evaporated to give 6.1 g of a crude product which was used without further purification.

LCMS (Method 3): Rₜ=0.53 min; MS (ESIpos) m/z=265 [M+H]⁺.

### Intermediate III-54

### 2-(6-Chloroimidazo[1,2-b]pyridazin-3-yl)-N-ethyl-N-(2-methoxyethyl)furo-[3,2-c]pyridin-4-amine

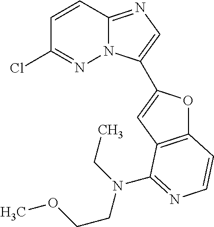 [SMILES: CCN(CCOC)c1nccc2oc(-c3cnc4ccc(Cl)nn34)cc12]

To 1.56 g (6.7 mmol) (3-bromo-6-chloroimidazo[1,2-b]pyridazine in 57 mL 1,4-dioxane were added 3 g (7 mmol) {4-[ethyl(2-methoxyethyl)amino]furo[3,2-c]pyridin-2-yl}boronic acid, 310 mg (0.27 mmol) tetrakis-(triphenylphosphin)palladium(0) and 10 mL of a 2 M aqueous solution of sodium carbonate. The mixture was stirred at 80° C. for 24 h.

Saturated aqueous ammonium chloride solution was added. The mixture was extracted with ethyl acetate. The organic layer was washed with brine, dried over sodium sulfate and evaporated. The precipitate was digested using 4 mL dichloromethane and 8 mL of n-hexane to give 1.2 g of the title compound as a crude product which was used without further purification.

LC-MS (Method 3): Rₜ=0.80 min; MS (ESIpos) m/z=372 [M+H]⁺.

### Intermediate III-55

### N-(2-tert-Butoxyethyl)furo[3,2-c]pyridin-4-amine

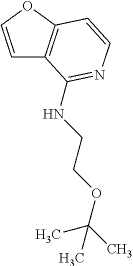 [SMILES: CC(C)(C)OCCNc1nccc2occc12]

A mixture of 4-chlorofuro[3,2-c]pyridine (1.7 g), 2-tert-butoxyethanamine hydrochloride (5.0 g) and Hünig base (5.6 mL) in 1-butanol (17 mL) was heated in a pressure tube to 120° C. for 72 h and to 150° C. for further 72 h. Water was added and the mixture was extracted with a mixture of ethyl acetate and hexane (3:1). The organic phase was washed with half-saturated ammonium chloride solution, dried (sodium sulfate) and the solvent was removed in vacuum. Silicagel chromatography gave 2.35 g of the title compound.

LCMS (Method 4): Rₜ=0.65 min; MS (ESIpos) m/z=235 [M+H]⁺.

### Intermediate III-56

### N-(2-tert-Butoxyethyl)-N-(furo[3,2-c]pyridin-4-yl)acetamide

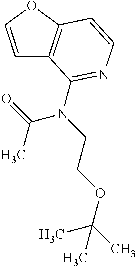 [SMILES: CC(=O)N(CCOC(C)(C)C)c1nccc2occc12]

To a stirred solution of N-(2-tert-butoxyethyl)furo[3,2-c]pyridin-4-amine (2.65 g) in dichloromethane (110 mL) was added Hünig base (3.9 mL) and pyridine (0.18 mL). The mixture was cooled to 0° C., acetyl chloride (1.4 mL) was added and the mixture was stirred at room temperature for 16 h. Water was added and the mixture was extracted with dichloromethane and methanol (100:1 mixture). The organic phase was washed with saturated sodium chloride solution, dried (sodium sulfate) and the solvent was removed in vacuum. Silicagel chromatography followed by aminophase silicagel chromatography gave 1.9 g of the title compound.

LCMS (Method 2): Rₜ=0.98 min; MS (ESIpos) m/z=277 [M+H]⁺.

### Intermediate III-57

### N-(2-tert-Butoxyethyl)-N-ethylfuro[3,2-c]pyridin-4-amine

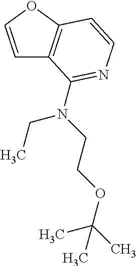 [SMILES: CCN(CCOC(C)(C)C)c1nccc2occc12]

To a stirred solution of N-(2-tert-butoxyethyl)-N-(furo[3,2-c]pyridin-4-yl)acetamide (1.90 g) in tetrahydrofurane (50 mL) was added borane dimethylsulfide complex (1.48 mL) at 0° C. The solution was allowed to warm up to room temperature, and was stirred at room temperature for 16 h. Water was added and the mixture was extracted with ethyl acetate. The organic phase was washed with saturated sodium chloride solution, dried (sodium sulfate) and the solvent was removed in vacuum. Silicagel chromatography gave 1.65 g of the title compound.

LCMS (Method 3): Rₜ=0.75 min; MS (ESIpos) m/z=263 [M+H]⁺.

### Intermediate III-58

### {4-[(2-tert-Butoxyethyl)(ethyl)amino]furo[3,2-c]pyridin-2-yl}boronic acid

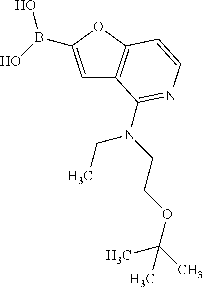 [SMILES: CCN(CCOC(C)(C)C)c1nccc2oc(B(O)O)cc12]

To a stirred solution of N-(2-tert-butoxyethyl)-N-ethylfuro[3,2-c]pyridin-4-amine (1.16 g) in anhydrous THF (11 mL) was added a solution of n-butyllithium in hexane (3.36 mL; c=2.5 M) at −78° C. The solution was stirred at −78° C. for 1.5 h. Triisopropyl borate (1.7 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added, the solution was extracted with a mixture of ethyl acetate and hexane (3:1) and the aqueous phase was lyophilized to give 1.2 g of the title compound as a crude product, which was used without further purification.

### Intermediate III-59

### [(2R)-1-(furo[3,2-c]pyridin-4-yl)pyrrolidin-2-yl]methanol

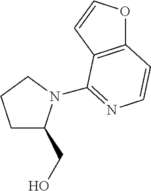 [SMILES: OC[C@H]1CCCN1c1nccc2occc12]

A mixture of 4-chlorofuro[3,2-c]pyridine (1.0 g), (2R)-pyrrolidin-2-ylmethanol (0.92 g) and Hünig base (2.2 mL) was heated to 160° C. in a microwave oven for 1 h. Water was added and the mixture was extracted with ethyl acetate. The organic phase was washed with half-saturated sodium chloride solution, dried (sodium sulfate) and the solvent was removed in vacuum. Silicagel chromatography gave 995 mg of the title compound.

LCMS (Method 5): Rₜ=0.92 min; MS (ESIpos) m/z=219 [M+H]⁺.

### Intermediate III-60

### 4-[(2R)-2-({[tert-Butyl(dimethyl)silyl]oxy}methyl)pyrrolidin-1-yl]furo[3,2-c]pyridine

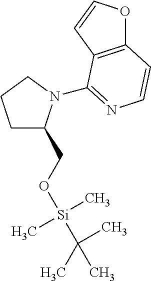 [SMILES: CC(C)(C)[Si](C)(C)OC[C@H]1CCCN1c1nccc2occc12]

To a stirred solution of [(2R)-1-(furo[3,2-c]pyridin-4-yl)pyrrolidin-2-yl]methanol (1850 mg) in THF (172 mL), triethylamine (3.54 mL), imidazole (865 mg) and tert-butyl(chloro)dimethylsilane (1.92 g) were added. The mixture was stirred at room temperature for 2 h. A half-saturated solution of sodium bicarbonate was added and the mixture was extracted with ethyl acetate. The organic phase was washed with half-saturated sodium chloride solution, dried (sodium sulfate) and the solvent was removed in vacuum. Aminophase silicagel chromatography gave 2.8 g of the title compound.

### Intermediate III-61

### {4-[(2R)-2-({[tert-Butyl(dimethyl)silyl]oxy}methyl)pyrrolidin-1-yl]furo[3,2-c]pyridin-2-yl}boronic acid

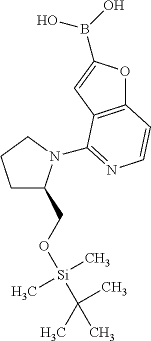 [SMILES: CC(C)(C)[Si](C)(C)OC[C@H]1CCCN1c1nccc2oc(B(O)O)cc12]

To a stirred solution of 4-[(2R)-2-({[tert-butyl(dimethyl)silyl]oxy}methyl)pyrrolidin-1-yl]furo[3,2-c]pyridine (2.9 g) in anhydrous THF (28 mL) was added a solution of n-butyllithium in hexane (5.2 mL; c=2.5 M) at −78° C. The solution was stirred at −78° C. for 1.5 h. Triisopropyl borate (2.7 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 2.8 g of the title compound as a crude product, which was used without further purification.

### Intermediate III-62

### 4-[(2R)-2-(Methoxymethyl)pyrrolidin-1-yl]furo[3,2-c]pyridine

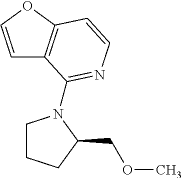 [SMILES: COC[C@H]1CCCN1c1nccc2occc12]

A mixture of 4-chlorofuro[3,2-c]pyridine (1.7 g) and (2R)-2-(methoxymethyl) pyrrolidine (2.5 g) was heated to 120° C. in a pressure tube for 28 h. Water was added and the mixture was extracted with a mixture of dichloromethane and methanol (100:1). The organic phase was washed with half-saturated sodium chloride solution, dried (sodium sulfate) and the solvent was removed in vacuum. Silicagel chromatography gave 2.2 g of the title compound.

LCMS (Method 2): Rₜ=0.54 min; MS (ESIpos) m/z=233 [M+H]⁺.

### Intermediate III-63

### {4-[(2R)-2-(Methoxymethyl)pyrrolidin-1-yl]furo[3,2-c]pyridin-2-yl}boronic acid

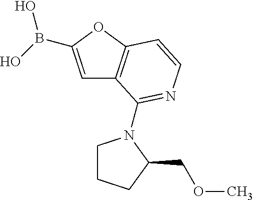 [SMILES: COC[C@H]1CCCN1c1nccc2oc(B(O)O)cc12]

To a stirred solution of 4-[(2R)-2-(methoxymethyl)pyrrolidin-1-yl]furo[3,2-c]pyridine (2.2 g) in anhydrous THF (24 mL) was added a solution of n-butyllithium in hexane (5.68 mL; c=2.5 M) at −78° C. The solution was stirred at −78° C. for 1.5 h. Triisopropyl borate (2.73 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 3.1 g of the title compound as a crude product (calculated purity: 84%), which was used without further purification.

### Intermediate III-64

### 4-[(2S)-2-(Methoxymethyl)pyrrolidin-1-yl]furo[3,2-c]pyridine

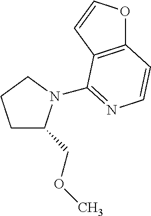 [SMILES: COC[C@@H]1CCCN1c1nccc2occc12]

A mixture of 4-chlorofuro[3,2-c]pyridine (1.7 g) and (2S)-2-(methoxymethyl) pyrrolidine (2.5 g) was heated to 120° C. in a pressure tube for 28 h. Water was added and the mixture was extracted with a mixture of dichloromethane and methanol (100:1). The organic phase was washed with half-saturated sodium chloride solution, dried (sodium sulfate) and the solvent was removed in vacuum. Silicagel chromatography gave 1.9 g of the title compound.

LCMS (Method 5): Rₜ=1.12 min; MS (ESIpos) m/z=233 [M+H]⁺.

### Intermediate III-65

### {4-[(2S)-2-(Methoxymethyl)pyrrolidin-1-yl]furo[3,2-c]pyridin-2-yl}boronic acid

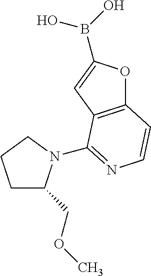 [SMILES: COC[C@@H]1CCCN1c1nccc2oc(B(O)O)cc12]

To a stirred solution of 4-[(2S)-2-(methoxymethyl)pyrrolidin-1-yl]furo[3,2-c]pyridine (1.81 g) in anhydrous THF (30 mL) was added a solution of n-butyllithium in hexane (4.68 mL; c=2.5 M) at −78° C. The solution was stirred at −78° C. for 1.5 h. Triisopropyl borate (2.24 g) was added at −78° C., the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 2.6 g of the title compound as a crude product (calculated purity: 82%), which was used without further purification.

### Intermediate III-66

### tert-Butyl [trans-3-({3-[4-(morpholin-4-yl)furo[3,2-c]pyridin-2-yl]imidazo-[1,2-b]pyridazin-6-yl}oxy)cyclobutyl]carbamate

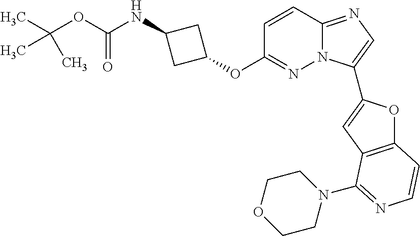 [SMILES: CC(C)(C)OC(=O)N[C@H]1C[C@H](Oc2ccc3ncc(-c4cc5c(N6CCOCC6)nccc5o4)n3n2)C1]

To a stirred suspension of tert-butyl (trans-3-hydroxycyclobutyl)carbamate (200 mg) in THF (6 mL) and DMF (0.6 mL) was added sodium hydride (60% w/w in oil; 43 mg) at 0° C. and the mixture was stirred at 0° C. for 30 minutes. 6-chloro-3-[4-(morpholin-4-yl)furo[3,2-c]pyridin-2-yl]imidazo[1,2-b]pyridazine (190 mg) was added and the mixture was stirred at room temperature for 16 hours. Water was added and the mixture was extracted with a mixture of dichloromethane and methanol (10:1). The organic phase was dried (sodium sulfate) and the solvent was removed in vacuum. Aminophase silicagel chromatography gave 190 mg of the title compound.

LCMS (Method 2): Rₜ=0.95 min; MS (ESIpos) m/z=507 [M+H]⁺.

### Intermediate III-67

### tert-Butyl [cis-3-({3-[4-(4-methylpiperazin-1-yl)furo[3,2-c]pyridin-2-yl]imidazo[1,2-b]pyridazin-6-yl}oxy)cyclobutyl]carbamate

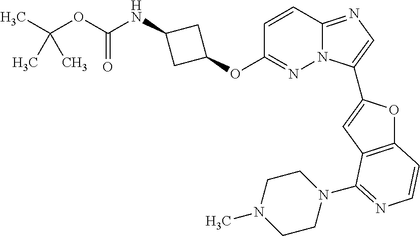 [SMILES: CN1CCN(c2nccc3oc(-c4cnc5ccc(O[C@H]6C[C@@H](NC(=O)OC(C)(C)C)C6)nn45)cc23)CC1]

To a stirred suspension of tert-butyl (trans-3-hydroxycyclobutyl)carbamate (152 mg) in THF (10 mL) and DMF (1.0 mL) was added sodium hydride (60% w/w in oil; 57 mg) at 0° C. and the mixture was stirred at room temperature for 30 minutes. 6-chloro-3-[4-(4-methylpiperazin-1-yl)furo[3,2-c]pyridin-2-yl]imidazo[1,2-b]pyridazine (150 mg) was added and the mixture was stirred at room temperature for 4 hours. Water was added and the mixture was extracted with a mixture of dichloromethane and methanol (10:1). The organic phase was dried (sodium sulfate) and the solvent was removed in vacuum. Silicagel chromatography gave a solid that was triturated with ether to give 180 mg of the title compound.

LCMS (Method 2): Rₜ=0.93 min; MS (ESIpos) m/z=520 [M+H]⁺.

### Intermediate III-68

### tert-Butyl (trans-3-{[3-(4-{methyl[3-(pyrrolidin-1-yl)propyl]amino}furo[3,2-c]pyridin-2-yl)imidazo[1,2-b]pyridazin-6-yl]oxy}cyclobutyl)carbamate

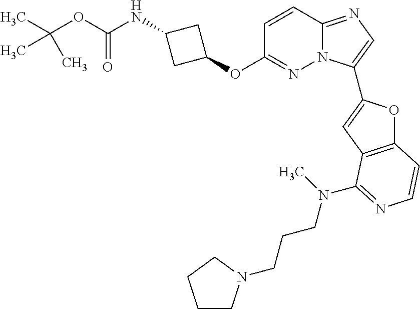 [SMILES: CN(CCCN1CCCC1)c1nccc2oc(-c3cnc4ccc(O[C@H]5C[C@H](NC(=O)OC(C)(C)C)C5)nn34)cc12]

To a stirred solution of tert-Butyl {trans-3-[(3-bromoimidazo[1,2-b]pyridazin-6-yl)oxy]cyclobutyl}-carbamate (150 mg) in 1-propanol (11 mL) was added 2M potassium carbonate solution (0.59 mL), crude (4-{methyl[3-(pyrrolidin-1-yl)propyl]amino}furo[3,2-c]pyridin-2-yl)boronic acid (52% w/w; 456 mg), triphenylphosphine (10.2 mg) and PdCl₂(PPh₃)₂ (27.5 mg). The mixture was heated to reflux for 1.5 h. Water was added and the mixture was extracted with a mixture of dichloromethane and methanol (4:1). The organic phase was dried (sodium sulfate) and the solvent was removed in vacuum. Silicagel chromatography gave 124 mg of the title compound.

LCMS (Method 5): Rₜ=1.69 min; MS (ESIpos) m/z=562 [M+H]⁺.

### Intermediate III-69

### tert-Butyl {trans-3-[(3-{4-[(3R)-3-(dimethylamino)pyrrolidin-1-yl]furo[3,2-c]pyridin-2-yl}imidazo[1,2-b]pyridazin-6-yl)oxy]cyclobutyl}carbamate

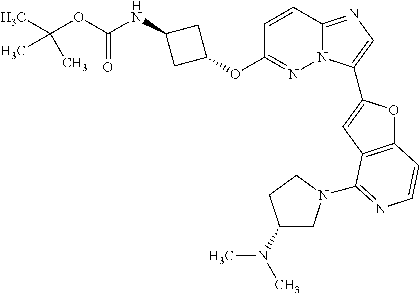 [SMILES: CN(C)[C@@H]1CCN(c2nccc3oc(-c4cnc5ccc(O[C@H]6C[C@H](NC(=O)OC(C)(C)C)C6)nn45)cc23)C1]

To a stirred solution of tert-Butyl {trans-3-[(3-bromoimidazo[1,2-b]pyridazin-6-yl)oxy]cyclobutyl}-carbamate (150 mg) in 1-propanol (11 mL) was added 2M potassium carbonate solution (0.59 mL), crude {4-[(3R)-3-(dimethylamino)pyrrolidin-1-yl]furo[3,2-c]pyridin-2-yl}boronic acid (65% w/w; 331 mg), triphenylphosphine (10.2 mg) and PdCl₂(PPh₃)₂ (27.5 mg). The mixture was heated to reflux for 1.5 h. Water was added and the mixture was extracted with a mixture of dichloromethane and methanol (4:1). The organic phase was dried (sodium sulfate) and the solvent was removed in vacuum. Silicagel chromatography gave 138 mg of the title compound.

LCMS (Method 5): Rₜ=1.25 min; MS (ESIpos) m/z=534 [M+H]⁺.

### Intermediate III-70

### tert-Butyl [trans-3-({3-[4-(4-tert-butylpiperazin-1-yl)furo[3,2-c]pyridin-2-yl]imidazo[1,2-b]pyridazin-6-yl}oxy)cyclobutyl]carbamate

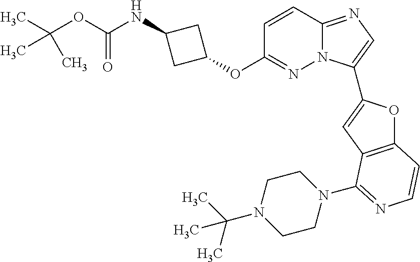 [SMILES: CC(C)(C)OC(=O)N[C@H]1C[C@H](Oc2ccc3ncc(-c4cc5c(N6CCN(C(C)(C)C)CC6)nccc5o4)n3n2)C1]

To a stirred solution of tert-Butyl {trans-3-[(3-bromoimidazo[1,2-b]pyridazin-6-yl)oxy]cyclobutyl}-carbamate (150 mg) in 1-propanol (13 mL) was added 2M potassium carbonate solution (0.59 mL), crude [4-(4-tert-butylpiperazin-1-yl)furo[3,2-c]pyridin-2-yl]boronic acid (55% w/w; 324 mg), triphenylphosphine (10.3 mg) and PdCl₂(PPh₃)₂ (28.0 mg). The mixture was heated to reflux for 2.0 h. A saturated solution of sodium bicarbonate was added and the mixture was extracted with a mixture of dichloromethane and methanol (100:1). The organic phase was dried (sodium sulfate) and the solvent was removed in vacuum. Silicagel chromatography gave a solid that was triturated with a mixture of ether and hexane to give 170 mg of the title compound.

LCMS (Method 5): Rₜ=1.46 min; MS (ESIpos) m/z=562 [M+H]⁺.

### Intermediate III-71

### tert-Butyl {trans-3-[(3-{4-[methyl(1-methylpiperidin-4-yl)amino]furo[3,2-c]pyridin-2-yl}imidazo[1,2-b]pyridazin-6-yl)oxy]cyclobutyl}carbamate

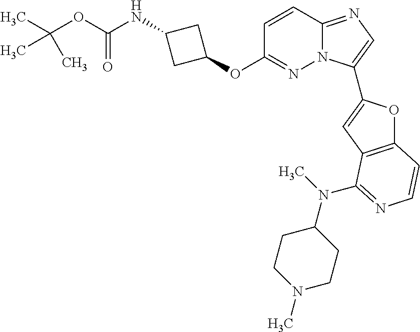 [SMILES: CN1CCC(N(C)c2nccc3oc(-c4cnc5ccc(O[C@H]6C[C@H](NC(=O)OC(C)(C)C)C6)nn45)cc23)CC1]

To a stirred solution of tert-Butyl {trans-3-[(3-bromoimidazo[1,2-b]pyridazin-6-yl)oxy]cyclobutyl}-carbamate (150 mg) in 1-propanol (11 mL) was added 2M potassium carbonate solution (0.59 mL), crude {4-[methyl(1-methylpiperidin-4-yl)amino]furo[3,2-c]pyridin-2-yl}boronic acid (26% w/w; 870 mg), triphenylphosphine (10.3 mg) and PdCl₂(PPh₃)₂ (27.5 mg). The mixture was heated to reflux for 1.5 h. Water was added and the mixture was extracted with a mixture of dichloromethane and methanol (4:1). The organic phase was dried (sodium sulfate) and the solvent was removed in vacuum. Silicagel chromatography gave 75 mg of the title compound.

LCMS (Method 5): Rₜ=1.38 min; MS (ESIpos) m/z=548 [M+H]⁺.

### Intermediate III-72

### tert-Butyl {trans-3-[(3-{4-[ethyl(2-methoxyethyl)amino]furo[3,2-c]pyridin-2-yl}imidazo[1,2-b]pyridazin-6-yl)oxy]cyclobutyl}carbamate

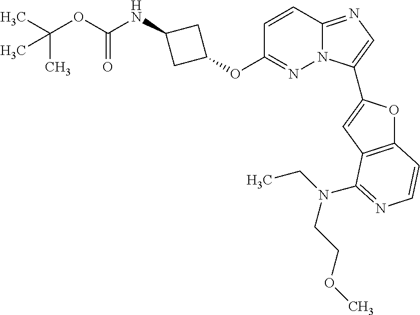 [SMILES: CCN(CCOC)c1nccc2oc(-c3cnc4ccc(O[C@H]5C[C@H](NC(=O)OC(C)(C)C)C5)nn34)cc12]

To a stirred solution of tert-Butyl {trans-3-[(3-bromoimidazo[1,2-b]pyridazin-6-yl)oxy]cyclobutyl}-carbamate (80 mg) in 1-propanol (7.0 mL) was added 2M potassium carbonate solution (0.31 mL), crude {4-[Ethyl(2-methoxyethyl)amino]furo[3,2-c]pyridin-2-yl}boronic acid (80% w/w; 137 mg), triphenylphosphine (5.5 mg) and PdCl₂(PPh₃)₂ (15.0 mg). The mixture was heated to reflux for 2.0 h. Water was added and the mixture was extracted with a mixture of dichloromethane and methanol (10:1). The organic phase was dried (sodium sulfate) and the solvent was removed in vacuum. Silicagel chromatography gave 80 mg of the title compound.

LCMS (Method 5): Rₜ=1.44 min; MS (ESIpos) m/z=523 [M+H]⁺.

### Intermediate III-73

### tert-Butyl {trans-3-[(3-{4-[(2R)-2-({[tert-butyl(dimethyl)silyl]oxy}methyl)pyrrolidin-1-yl]furo[3,2-c]pyridin-2-yl}imidazo[1,2-b]pyridazin-6-yl)oxy]cyclobutyl}carbamate

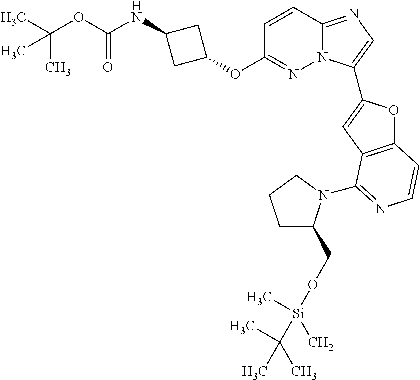 [SMILES: CC(C)(C)OC(=O)N[C@H]1C[C@H](Oc2ccc3ncc(-c4cc5c(N6CCC[C@@H]6CO[Si](C)(C)C(C)(C)C)nccc5o4)n3n2)C1]

To a stirred solution of tert-Butyl {trans-3-[(3-bromoimidazo[1,2-b]pyridazin-6-yl)-oxy]cyclobutyl}-carbamate (200 mg) in 1-propanol (14 mL) was added 2M potassium carbonate solution (0.78 mL), crude {4-[Ethyl(2-methoxyethyl)amino] furo[3,2-c]pyridin-2-yl}boronic acid (480 mg), triphenylphosphine (13.7 mg) and PdCl₂(PPh₃)₂ (36.6 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 270 mg of the title compound.

LCMS (Method 5): Rₜ=1.82 min; MS (ESIpos) m/z=635 [M+H]⁺.

### Intermediate III-74

### tert-Butyl {trans-3-[(3-{4-[(2R)-2-(hydroxymethyl)pyrrolidin-1-yl]furo[3,2-c]pyridin-2-yl}imidazo[1,2-b]pyridazin-6-yl)oxy]cyclobutyl}carbamate

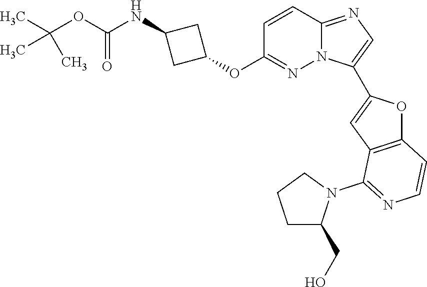 [SMILES: CC(C)(C)OC(=O)N[C@H]1C[C@H](Oc2ccc3ncc(-c4cc5c(N6CCC[C@@H]6CO)nccc5o4)n3n2)C1]

To a stirred solution of tert-butyl {trans-3-[(3-{4-[(2R)-2-({[tert-butyl(dimethyl)-silyl]oxy}methyl) pyrrolidin-1-yl]furo[3,2-c]pyridin-2-yl}imidazo[1,2-b]pyridazin-6-yl)oxy]cyclobutyl}carbamate (270 mg) in THF (25 mL) was added a solution of TBAF in THF (0.85 mL; c=1.0 mol/L). The mixture was stirred at room temperature for 4 h. A half-saturated solution of sodium bicarbonate was added and the mixture was extracted with a mixture of ethyl acetate and methanol. The organic phase was dried (sodium sulfate) and the solvent was removed in vacuum. Silicagel chromatography gave a solid that was triturated with a mixture of hexane and dichloromethane to give 132 mg of the title compound.

LCMS (Method 5): Rₜ=1.26 min; MS (ESIpos) m/z=521 [M+H]⁺.

### Intermediate III-75

### tert-Butyl 4-{2-[6-({trans-3-[(tert-butoxycarbonyl)amino]cyclobutyl}oxy)imidazo[1,2-b]pyridazin-3-yl]furo[3,2-c]pyridin-4-yl}piperazine-1-carboxylate

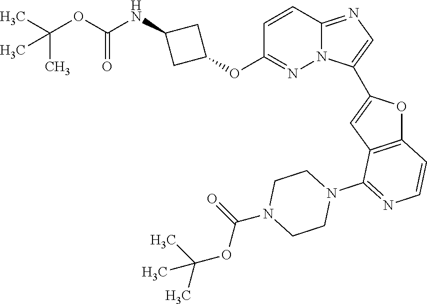 [SMILES: CC(C)(C)OC(=O)N[C@H]1C[C@H](Oc2ccc3ncc(-c4cc5c(N6CCN(C(=O)OC(C)(C)C)CC6)nccc5o4)n3n2)C1]

To a stirred solution of tert-Butyl {trans-3-[(3-bromoimidazo[1,2-b]pyridazin-6-yl)oxy]cyclobutyl}carbamate (80 mg) in 1-propanol (7.0 mL) was added 2M potassium carbonate solution (0.31 mL), crude {4-[4-(tert-butoxycarbonyl)piperazin-1-yl]furo[3,2-c]pyridin-2-yl}boronic acid (80% w/w; 181 mg), triphenylphosphine (5.5 mg) and PdCl₂(PPh₃)₂ (15.0 mg). The mixture was heated to reflux for 2.0 h. The reaction mixture was filtered through an aminophase silicagel column and the solvent was removed in vacuum. Silicagel chromatography gave a solid that was triturated with a mixture of ethyl acetate and hexane to give 90 mg of the title compound.

LCMS (Method 5): Rₜ=1.51 min; MS (ESIpos) m/z=606 [M+H]⁺.

### Intermediate III-76

### tert-Butyl {trans-3-[(3-{4-[(2R,6S)-2,6-dimethylmorpholin-4-yl]furo[3,2-c]pyridin-2-yl}imidazo[1,2-b]pyridazin-6-yl)oxy]cyclobutyl}carbamate

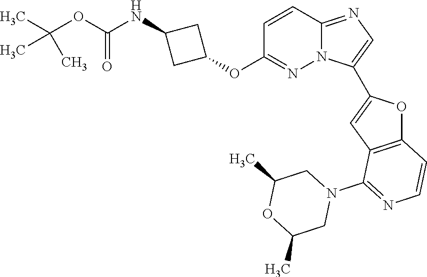 [SMILES: C[C@@H]1CN(c2nccc3oc(-c4cnc5ccc(O[C@H]6C[C@H](NC(=O)OC(C)(C)C)C6)nn45)cc23)C[C@H](C)O1]

To a stirred solution of tert-Butyl {trans-3-[(3-bromoimidazo[1,2-b]pyridazin-6-yl)oxy]cyclobutyl}carbamate (150 mg) in 1-propanol (11 mL) was added 2M potassium carbonate solution (0.59 mL), crude {4-[(2R,6S)-2,6-dimethylmorpholin-4-yl]furo[3,2-c]pyridin-2-yl}boronic acid (75% w/w; 288 mg), triphenylphosphine (10.2 mg) and PdCl₂(PPh₃)₂ (27.5 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 178 mg of the title compound.

LCMS (Method 5): Rₜ=1.41 min; MS (ESIpos) m/z=535 [M+H]⁺.

### Intermediate III-77

### tert-Butyl {trans-3-[(3-{4-[(3-{[tert-butyl(dimethyl)silyl]oxy}propyl)(methyl)amino]furo[3,2-c]pyridin-2-yl}imidazo[1,2-b]pyridazin-6-yl)oxy]cyclobutyl}carbamate

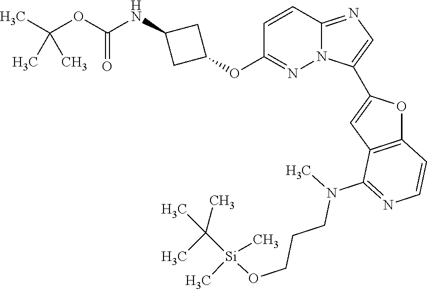 [SMILES: CN(CCCO[Si](C)(C)C(C)(C)C)c1nccc2oc(-c3cnc4ccc(O[C@H]5C[C@H](NC(=O)OC(C)(C)C)C5)nn34)cc12]

To a stirred solution of tert-Butyl {trans-3-[(3-bromoimidazo[1,2-b]pyridazin-6-yl)oxy]cyclobutyl}-carbamate (150 mg) in 1-propanol (11 mL) was added 2M potassium carbonate solution (0.59 mL), crude {4-[(3-{[tert-butyl(dimethyl)-silyl]oxy}propyl)(methyl)amino]furo[3,2-c]pyridin-2-yl}boronic acid (468 mg), triphenylphosphine (10.2 mg) and PdCl₂(PPh₃)₂ (27.5 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with a mixture of dichloromethane and hexane to give 142 mg of the title compound.

LCMS (Method 5): Rₜ=1.78 min; MS (ESIpos) m/z=623 [M+H]⁺.

### Intermediate III-78

### tert-Butyl {trans-3-[(3-{4-[(3-hydroxypropyl)(methyl)amino]furo[3,2-c]pyridin-2-yl}imidazo[1,2-b]pyridazin-6-yl)oxy]cyclobutyl}carbamate

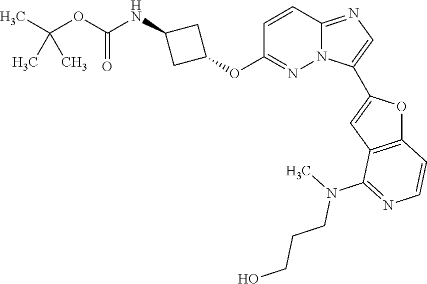 [SMILES: CN(CCCO)c1nccc2oc(-c3cnc4ccc(O[C@H]5C[C@H](NC(=O)OC(C)(C)C)C5)nn34)cc12]

To a stirred solution of tert-butyl {trans-3-[(3-{4-[(3-{[tert-butyl(dimethyl)silyl]-oxy}propyl)(methyl)amino]furo[3,2-c]pyridin-2-yl}imidazo[1,2-b]pyridazin-6-yl)oxy]-cyclobutyl}carbamate (130 mg) in THF (10 mL) was added a solution of TBAF in THF (0.42 mL; c=1.0 mol/L). The mixture was stirred at room temperature for 4 h. A half-saturated solution of sodium bicarbonate was added and the mixture was extracted with a mixture of ethyl acetate and methanol. The organic phase was dried (sodium sulfate) and the solvent was removed in vacuum. Silicagel chromatography gave a solid that was triturated with a mixture of dichloromethane and hexane to give 46 mg of the title compound.

LCMS (Method 5): Rₜ=1.22 min; MS (ESIpos) m/z=509 [M+H]⁺.

### Intermediate III-79

### tert-Butyl {trans-3-[(3-{4-[(2-tert-butoxyethyl)(ethyl)amino]furo[3,2-c]pyridin-2-yl}imidazo[1,2-b]pyridazin-6-yl)oxy]cyclobutyl}carbamate

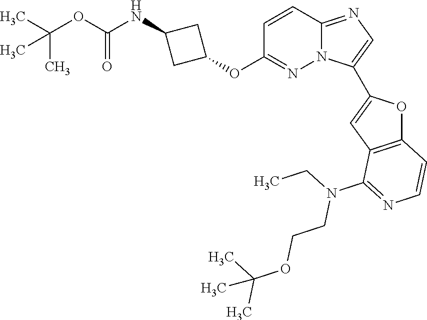 [SMILES: CCN(CCOC(C)(C)C)c1nccc2oc(-c3cnc4ccc(O[C@H]5C[C@H](NC(=O)OC(C)(C)C)C5)nn34)cc12]

To a stirred solution of tert-Butyl {trans-3-[(3-bromoimidazo[1,2-b]pyridazin-6-yl)oxy]cyclobutyl}-carbamate (100 mg) in 1-propanol (10 mL) was added 2M potassium carbonate solution (0.39 mL), crude {4-[(2-tert-butoxyethyl)(ethyl)amino]furo[3,2-c]pyridin-2-yl}boronic acid (199 mg), triphenylphosphine (6.8 mg) and PdCl₂(PPh₃)₂ (18.7 mg). The mixture was heated to reflux for 2 h. The warm mixture was filtered, the solvent was removed in vacuum. Silicagel chromatography followed by aminophase silicagel chromatography gave 120 mg of the title compound.

LCMS (Method 5): Rₜ=1.58 min; MS (ESIpos) m/z=565 [M+H]⁺.

### Intermediate III-80

### N-(3-{[tert-Butyl(dimethyl)silyl]oxy}propyl)-N-methyl-2-{6-[3-(methylsulfonyl)-propoxy]imidazo[1,2-b]pyridazin-3-yl}furo[3,2-c]pyridin-4-amine

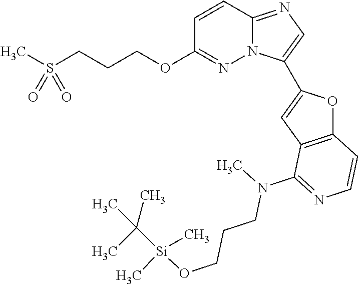 [SMILES: CN(CCCO[Si](C)(C)C(C)(C)C)c1nccc2oc(-c3cnc4ccc(OCCCS(C)(=O)=O)nn34)cc12]

To a stirred solution of 3-Bromo-6-[3-(methylsulfonyl)propoxy]imidazo[1,2-b]pyridazine (100 mg) in 1-propanol (10 mL) was added 2M potassium carbonate solution (0.45 mL), crude {4-[(3-{[tert-butyl(dimethyl)silyl]oxy}propyl) (methyl)amino]furo[3,2-c]pyridin-2-yl}boronic acid (357 mg), triphenylphosphine (7.8 mg) and PdCl₂(PPh₃)₂ (21.0 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with a mixture of dichloromethane and hexane to give 138 mg of the title compound.

LCMS (Method 5): Rₜ=1.52 min; MS (ESIpos) m/z=574 [M+H]⁺.

### Intermediate III-81

### 2-(6-{[(2S)-2-Aminopropyl]oxy}imidazo[1,2-b]pyridazin-3-yl)-N-(3-{[tert-butyl-(dimethyl)silyl]oxy}propyl)-N-methylfuro[3,2-c]pyridin-4-amine

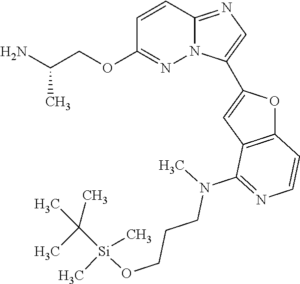 [SMILES: C[C@H](N)COc1ccc2ncc(-c3cc4c(N(C)CCCO[Si](C)(C)C(C)(C)C)nccc4o3)n2n1]

To a stirred solution of (2S)-1-[(3-Bromoimidazo[1,2-b]pyridazin-6-yl)oxy]propan-2-amine (100 mg) in 1-propanol (10 mL) was added 2M potassium carbonate solution (0.55 mL), crude {4-[(3-{[tert-butyl(dimethyl)silyl]oxy}propyl) (methyl)amino]furo-[3,2-c]pyridin-2-yl}boronic acid (441 mg), triphenylphosphine (9.7 mg) and PdCl₂(PPh₃)₂ (25.9 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with a mixture of dichloromethane and hexane to give 170 mg of the title compound.

LCMS (Method 5): Rₜ=1.60 min; MS (ESIpos) m/z=511 [M+H]⁺.

### Intermediate III-82

### (5R)-5-{[(3-{4-[(3-{[tert-Butyl(dimethyl)silyl]oxy}propyl)(methyl)amino]furo[3,2-c]pyridin-2-yl}imidazo[1,2-b]pyridazin-6-yl)oxy]methyl}pyrrolidin-2-one

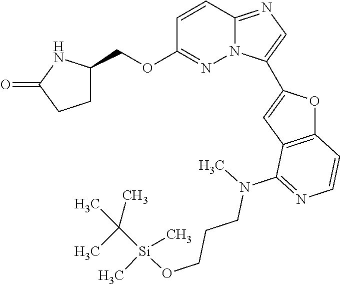 [SMILES: CN(CCCO[Si](C)(C)C(C)(C)C)c1nccc2oc(-c3cnc4ccc(OC[C@H]5CCC(=O)N5)nn34)cc12]

To a stirred solution of (5R)-5-{[(3-Bromoimidazo[1,2-b]pyridazin-6-yl)oxy]methyl}pyrrolidin-2-one (100 mg) in 1-propanol (9 mL) was added 2M potassium carbonate solution (0.48 mL), crude {4-[(3-{[tert-butyl(dimethyl)silyl]oxy}propyl)(methyl)amino]furo[3,2-c]pyridin-2-yl}boronic acid (384 mg), triphenylphosphine (8.4 mg) and PdCl₂(PPh₃)₂ (22.6 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with a mixture of dichloromethane and hexane to give 143 mg of the title compound.

LCMS (Method 5): Rₜ=1.48 min; MS (ESIpos) m/z=551 [M+H]⁺.

### Intermediate III-83

### 3-{4-[4-({[tert-Butyl(dimethyl)silyl]oxy}methyl)piperidin-1-yl]furo[3,2-c]pyridin-2-yl}-6-[3-(methylsulfonyl)propoxy]imidazo[1,2-b]pyridazine

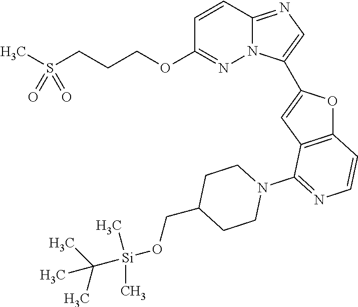 [SMILES: CC(C)(C)[Si](C)(C)OCC1CCN(c2nccc3oc(-c4cnc5ccc(OCCCS(C)(=O)=O)nn45)cc23)CC1]

To a stirred solution of 3-Bromo-6-[3-(methylsulfonyl)propoxy]imidazo[1,2-b]pyridazine (80 mg) in 1-propanol (8 mL) was added 2M potassium carbonate solution (0.36 mL), crude {4-[4-({[tert-butyl(dimethyl)silyl]oxy}methyl)piperidin-1-yl]furo[3,2-c]pyridin-2-yl}boronic acid (227 mg), triphenylphosphine (6.3 mg) and PdCl₂(PPh₃)₂ (17.1 mg). The mixture was heated to reflux for 2 h. The reaction mixture was filtered through an aminophase silicagel column and the solvent was removed in vacuum. Aminophase silicagel chromatography gave 130 mg of the title compound.

LCMS (Method 5): Rₜ=1.62 min; MS (ESIpos) m/z=600 [M+H]⁺.

### Intermediate III-84

### (2S)-1-[(3-{4-[4-({[tert-Butyl(dimethyl)silyl]oxy}methyl)piperidin-1-yl]furo-[3,2-c]pyridin-2-yl}imidazo[1,2-b]pyridazin-6-yl)oxy]propan-2-amine

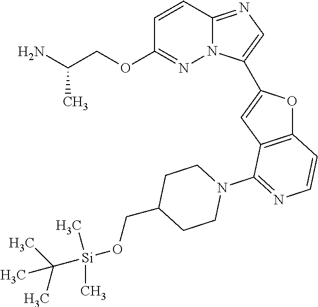 [SMILES: C[C@H](N)COc1ccc2ncc(-c3cc4c(N5CCC(CO[Si](C)(C)C(C)(C)C)CC5)nccc4o3)n2n1]

To a stirred solution of (2S)-1-[(3-Bromoimidazo[1,2-b]pyridazin-6-yl)oxy]propan-2-amine (80 mg) in 1-propanol (10 mL) was added 2M potassium carbonate solution (0.44 mL), crude {4-[4-({[tert-butyl(dimethyl)silyl]oxy}methyl) piperidin-1-yl]furo[3,2-c]pyridin-2-yl}boronic acid (280 mg), triphenylphosphine (7.7 mg) and PdCl₂(PPh₃)₂ (21.1 mg). The mixture was heated to reflux for 2 h. The reaction mixture was filtered through an aminophase silicagel column and the solvent was removed in vacuum. Aminophase silicagel chromatography gave 140 mg of the title compound.

LCMS (Method 5): Rₜ=1.72 min; MS (ESIpos) m/z=537 [M+H]⁺.

### Intermediate III-85

### (2R)-1-[(3-{4-[4-({[tert-Butyl(dimethyl)silyl]oxy}methyl)piperidin-1-yl]furo-[3,2-c]pyridin-2-yl}imidazo[1,2-b]pyridazin-6-yl)oxy]propan-2-amine

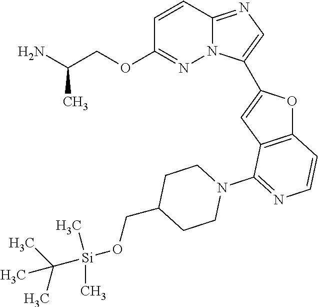 [SMILES: C[C@@H](N)COc1ccc2ncc(-c3cc4c(N5CCC(CO[Si](C)(C)C(C)(C)C)CC5)nccc4o3)n2n1]

To a stirred solution of (2R)-1-[(3-Bromoimidazo[1,2-b]pyridazin-6-yl)oxy]propan-2-amine (45 mg) in 1-propanol (6 mL) was added 2M potassium carbonate solution (0.25 mL), crude {4-[4-({[tert-butyl(dimethyl)silyl]oxy}methyl) piperidin-1-yl]furo[3,2-c]pyridin-2-yl}boronic acid (110 mg), triphenylphosphine (4.4 mg) and PdCl₂(PPh₃)₂ (11.9 mg). The mixture was heated to reflux for 2 h. The reaction mixture was filtered through an aminophase silicagel column and the solvent was removed in vacuum. Aminophase silicagel chromatography gave 75 mg of the title compound.

LCMS (Method 5): Rₜ=1.66 min; MS (ESIpos) m/z=537 [M+H]⁺.

### Intermediate III-86

### (5R)-5-{[(3-{4-[4-({[tert-Butyl(dimethyl)silyl]oxy}methyl)piperidin-1-yl]furo-[3,2-c]pyridin-2-yl}imidazo[1,2-b]pyridazin-6-yl)oxy]methyl}pyrrolidin-2-one

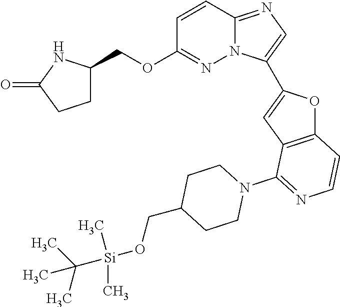 [SMILES: CC(C)(C)[Si](C)(C)OCC1CCN(c2nccc3oc(-c4cnc5ccc(OC[C@H]6CCC(=O)N6)nn45)cc23)CC1]

To a stirred solution of (5R)-5-{[(3-Bromoimidazo[1,2-b]pyridazin-6-yl)oxy]methyl}pyrrolidin-2-one (80 mg) in 1-propanol (9 mL) was added 2M potassium carbonate solution (0.39 mL), crude {4-[4-({[tert-butyl(dimethyl)silyl]oxy}methyl)piperidin-1-yl]furo[3,2-c]pyridin-2-yl}boronic acid (244 mg), triphenylphosphine (6.7 mg) and PdCl₂(PPh₃)₂ (18.4 mg). The mixture was heated to reflux for 2 h. The reaction mixture was filtered through an aminophase silicagel column and the solvent was removed in vacuum. Aminophase silicagel chromatography gave 130 mg of the title compound.

LCMS (Method 5): Rₜ=1.59 min; MS (ESIpos) m/z=577 [M+H]⁺.

### Intermediate III-87

### (2S)-1-[(3-{4-[(2R)-2-({[tert-Butyl(dimethyl)silyl]oxy}methyl)pyrrolidin-1-yl]furo[3,2-c]pyridin-2-yl}imidazo[1,2-b]pyridazin-6-yl)oxy]propan-2-amine

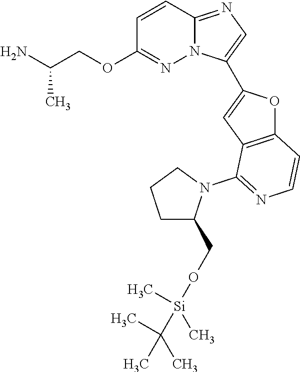 [SMILES: C[C@H](N)COc1ccc2ncc(-c3cc4c(N5CCC[C@@H]5CO[Si](C)(C)C(C)(C)C)nccc4o3)n2n1]

To a stirred solution of (2S)-1-[(3-Bromoimidazo[1,2-b]pyridazin-6-yl)oxy]propan-2-amine (150 mg) in 1-propanol (15 mL) was added 2M potassium carbonate solution (0.83 mL), crude {4-[(2R)-2-({[tert-Butyl(dimethyl)silyl]oxy}methyl)pyrrolidin-1-yl]furo[3,2-c]pyridin-2-yl}boronic acid (509 mg), triphenylphosphine (14.5 mg) and PdCl₂(PPh₃)₂ (38.8 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 159 mg of the title compound.

LCMS (Method 5): Rₜ=1.65 min; MS (ESIpos) m/z=523 [M+H]⁺.

### Intermediate III-88

### (6R)-4-(2,2-Dimethylpropanoyl)-6-(hydroxymethyl)piperazin-2-one

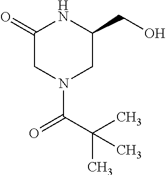

(6R)-4-(2,2-Dimethylpropanoyl)-6-(hydroxymethyl)piperazin-2-one was prepared as described in Organic Letters, 2004, Vol. 6, pages 4096-4072.

### Intermediate III-89

### (6S)-4-(2,2-Dimethylpropanoyl)-6-(hydroxymethyl)piperazin-2-one

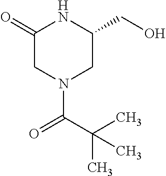

(6S)-4-(2,2-dimethylpropanoyl)-6-(hydroxymethyl)piperazin-2-one was prepared as described in Organic Letters, 2004, Vol. 6, pages 4096-4072.

### Intermediate III-90

### 4-[(3R)-3-Methylmorpholin-4-yl]furo[3,2-c]pyridine

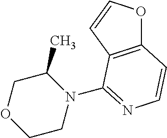 [SMILES: C[C@@H]1COCCN1c1nccc2occc12]

4 g (26 mmol) 4-chlorofuro[3,2-c]pyridine, 4 g (39 mmol) (R)-3-methylmorpholine, 9 mL (52 mmol) N-ethyl-N-isopropylpropan-2-amine and 160 mg (1.3 mmol) N,N-dimethylpyridin-4-amine were heated to 180° C. for 6 h in a microwave oven.

The crude product was purified by flash chromatography to give 3.9 g of the title compound.

¹H-NMR (400 MHz, DMSO-d₆), δ [ppm]=1.10-1.16 (3H), 3.23-3.33 (1H), 3.51 (1H), 3.63-3.72 (2H), 3.87-4.00 (2H), 4.46 (1H), 6.97 (1H), 7.12 (1H), 7.89 (1H), 7.94 (1H).

LCMS (Method 3): Rₜ=0.49 min; MS (ESIpos) m/z=219 [M+H]⁺.

### Intermediate III-91

### {4-[(3R)-3-Methylmorpholin-4-yl]furo[3,2-c]pyridin-2-yl}boronic acid

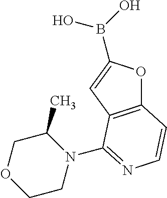 [SMILES: C[C@@H]1COCCN1c1nccc2oc(B(O)O)cc12]

1.3 g (6 mmol) 4-[(3R)-3-methylmorpholin-4-yl]furo[3,2-c]pyridine in 62 mL anhydrous THF was cooled to −78° C. 3.6 mL (9 mmol) of a 2.5 M solution of n-butyl lithium in hexane was added. The mixture was stirred for 90 min at −78° C. 2.1 mL (9 mmol) of triisopropyl borate was added at −78° C. The cooling bath was removed and the mixture was stirred at room temperature for 19 h.

A small amount of water was added and the solvent was evaporated to give 2.5 g of a crude product which was used without further purification.

LCMS (Method 3): Rₜ=0.47 min; MS (ESIpos) m/z=263 [M+H]⁺.

### Intermediate III-92

### 4-[(3S)-3-Methylmorpholin-4-yl]furo[3,2-c]pyridine

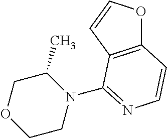 [SMILES: C[C@H]1COCCN1c1nccc2occc12]

1.5 g (9.8 mmol) 4-chlorofuro[3,2-c]pyridine, 2 g (14.7 mmol) (S)-3-methylmorpholine, 8 mL (46 mmol) N-ethyl-N-isopropylpropan-2-amine and 60 mg (0.49 mmol) N,N-dimethylpyridin-4-amine were heated to 180° C. for 6 h in a microwave oven.

The crude product was purified by flash chromatography to give 1.6 g of the title compound.

¹H-NMR (300 MHz, DMSO-d₆), δ [ppm]=1.13 (3H), 3.21-3.28 (1H), 3.51 (1H), 3.61-3.72 (2H), 3.85-4.01 (2H), 4.46 (1H), 6.97 (1H), 7.13 (1H), 7.90 (1H), 7.93 (1H)

LCMS (Method 3): Rₜ=0.49 min; MS (ESIpos) m/z=219 [M+H]⁺.

### Intermediate III-93

### {4-[(3S)-3-Methylmorpholin-4-yl]furo[3,2-c]pyridin-2-yl}boronic acid

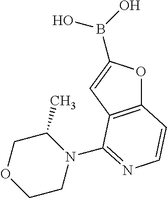 [SMILES: C[C@H]1COCCN1c1nccc2oc(B(O)O)cc12]

1.6 g (7.4 mmol) 4-[(3S)-3-methylmorpholin-4-yl]furo[3,2-c]pyridine in 77 mL anhydrous THF was cooled to −78° C. 4.5 mL (11 mmol) of a 2.5 M solution of n-butyl lithium in hexane was added. The mixture was stirred for 90 min at −78° C. 2.6 mL (11 mmol) of triisopropyl borate was added at −78° C. The cooling bath was removed and the mixture was stirred at room temperature for 20 h.

Water was added. The mixture was concentrated to give 3.1 g of a crude product which was used without further purification.

LCMS (Method 4): Rₜ=0.46 min; MS (ESIpos) m/z=263 [M+H]⁺.

### Intermediate III-94

### 4-[(2S)-2-Methylmorpholin-4-yl]furo[3,2-c]pyridine

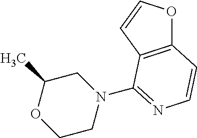 [SMILES: C[C@H]1CN(c2nccc3occc23)CCO1]

1.5 g (9.8 mmol) 4-chlorofuro[3,2-c]pyridine, 1.5 g (14.7 mmol) (S)-2-methylmorpholine hydrochloride, 8 mL (46 mmol) N-ethyl-N-isopropylpropan-2-amine and 60 mg (0.49 mmol) N,N-dimethylpyridin-4-amine were heated to 180° C. for 4 h in a microwave oven.

The crude product was purified by flash chromatography to give 1.6 g of the title compound.

¹H-NMR (300 MHz, DMSO-d₆), δ [ppm]=1.11-1.21 (3H), 2.66 (1H), 3.00 (1H), 3.56-3.70 (2H), 3.86-3.95 (1H), 4.06-4.23 (2H), 7.05 (1H), 7.20 (1H), 7.93-7.99 (2H).

LCMS (Method 3): Rₜ=0.52 min; MS (ESIpos) m/z=219 [M+H]⁺.

### Intermediate III-95

### {4-[(2S)-2-Methylmorpholin-4-yl]furo[3,2-c]pyridin-2-yl}boronic acid

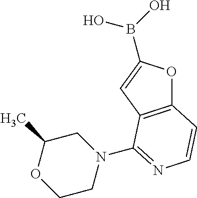 [SMILES: C[C@H]1CN(c2nccc3oc(B(O)O)cc23)CCO1]

1.9 g (8.5 mmol) 4-[(2S)-2-methylmorpholin-4-yl]furo[3,2-c]pyridine in 88 mL anhydrous THF was cooled to −78° C. 5.1 mL (12.8 mmol) of a 2.5 M solution of n-butyl lithium in hexane was added. The mixture was stirred for 90 min at −78° C. 2.95 mL (12.8 mmol) of triisopropyl borate was added at −78° C. The cooling bath was removed and the mixture was stirred at room temperature for 25 h.

A small amount of water was added and the solvent was evaporated to give 3.3 g of a crude product which was used without further purification.

LCMS (Method 7): Rₜ=0.46 min; MS (ESIpos) m/z=263 [M+H]⁺.

### Intermediate III-96

### 4-(4-Phenylpiperazin-1-yl)furo[3,2-c]pyridine

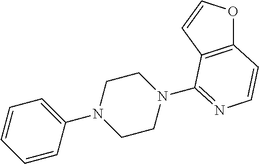 [SMILES: c1ccc(N2CCN(c3nccc4occc34)CC2)cc1]

3 g (19.5 mmol) 4-chlorofuro[3,2-c]pyridine and 15 mL (98 mmol) N-phenylpiperazine were heated to 180° C. for 2 h in a microwave oven.

Ethyl acetate was added. The obtained mixture was washed with water and half-saturated brine, dried over sodium sulfate and evaporated. The crude product was purified by flash chromatography to give 5.8 g of the title compound.

¹H-NMR (300 MHz, DMSO-d₆), δ [ppm]=3.21-3.37 (4H), 3.70-3.80 (4H), 6.77 (1H), 6.95 (2H), 7.04 (1H), 7.14-7.27 (3H), 7.90-8.01 (2H).

LCMS (Method 3): Rₜ=0.78 min; MS (ESIpos) m/z=280[M+H]⁺.

### Intermediate III-97

### [4-(4-Phenylpiperazin-1-yl)furo[3,2-c]pyridin-2-yl]boronic acid

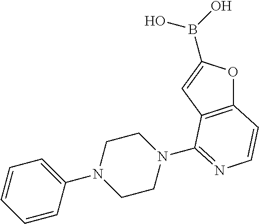 [SMILES: OB(O)c1cc2c(N3CCN(c4ccccc4)CC3)nccc2o1]

5.4 g (19.5 mmol) 4-(4-phenylpiperazin-1-yl)furo[3,2-c]pyridine in 202 mL anhydrous THF was cooled to −78° C. 11.7 mL (29.3 mmol) of a 2.5 M solution of n-butyl lithium in hexane was added. The mixture was stirred for 90 min at −78° C. 6.8 mL (29.3 mmol) of triisopropyl borate was added at −78° C. The cooling bath was removed and the mixture was stirred at room temperature for 20 h.

A small amount of water was added and the solvent was evaporated to give 7.6 g of a crude product which was used without further purification.

LCMS (Method 3): Rₜ=0.7 min; MS (ESIpos) m/z=324 [M+H]⁺.

### Intermediate III-98

### 3-Bromo-6-(piperidin-2-ylmethoxy)imidazo[1,2-b]pyridazine

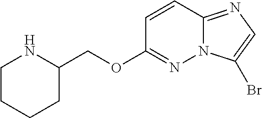 [SMILES: Brc1cnc2ccc(OCC3CCCCN3)nn12]

To a stirred suspension of piperidin-2-ylmethanol (4.84 g) in anhydrous THF (200 mL) and anhydrous DMF (20 mL) was added sodium hydride (60% w/w in oil; 1.66 g) at 0° C. and the mixture was stirred at 0° C. for 30 minutes. 3-Bromo-6-chloroimidazo[1,2-b]pyridazine (3.71 g) was added and the mixture was stirred at room temperature for 16 hours. Water was added and the mixture was extracted with ethyl acetate. The organic phase was dried (sodium sulfate) and the solvent was removed in vacuum. Silicagel chromatography gave a solid that was triturated with hexane to give 2.6 g of the title compound.

LCMS (Method 2): Rₜ=0.52 min; MS (ESIpos) m/z=311; 313 [M+H]⁺.

### Intermediate IV-1

### 3-Bromo-6-chloro-imidazo[1,2-b]pyridazine

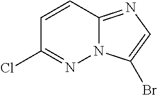 [SMILES: Clc1ccc2ncc(Br)n2n1]

3-Bromo-6-chloro-imidazo[1,2-b]pyridazine was synthesized as described for example in WO 2007/147646 or DE 10 2006 029447, e.g. as follows:

### Step 1: Preparation of 6-Chloroimidazo[1,2-b]pyridazine

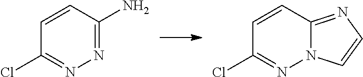 [SMILES: Nc1ccc(Cl)nn1 ; Clc1ccc2nccn2n1]

5.0 g (38.6 mmol) of 3-amino-6-chloropyridazine were heated together with 4.7 mL (40 mmol) of chloroacetaldehyde (55% strength in water) in 15 mL of n-butanol at 120° C. for a period of 5 days. After the reaction was complete, the reaction mixture was added to saturated sodium bicarbonate solution and extracted three times with ethyl acetate. The combined organic phases were then washed with sat. sodium chloride solution and dried over sodium sulfate, and the solvent was removed in vacuo. In the final purification by chromatography on silica gel, 4.17 g (70%) of the desired product were isolated in the form of an amorphous white solid.

¹H-NMR (CHLOROFORM-d): δ [ppm]=7.06 (d, 1H); 7.79 (d, 1H); 7.92, (d, 1H); 7.96 (d, 1H).

### Step 2: Preparation of 3-Bromo-6-chloroimidazo[1,2-b]pyridazine

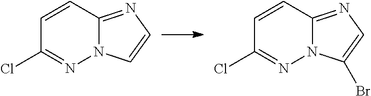 [SMILES: Clc1ccc2nccn2n1 ; Clc1ccc2ncc(Br)n2n1]

478 mg (3.11 mmol) of 6-chloroimidazo[1,2-b]pyridazine were introduced into 10 mL of chloroform under argon and, while cooling in ice, 664 mg (3.73 mmol) of N-bromo-succinimide were added. After the addition was complete, the reaction mixture was stirred at room temperature overnight. The reaction mixture was then mixed with water and ethyl acetate and, after addition of saturated sodium bicarbonate solution, the phases were separated. The aqueous phase was extracted three more times with ethyl acetate. The combined organic phases were then washed with saturated sodium chloride solution and dried over sodium sulfate. In the final removal of the solvent in vacuo, the desired product was isolated in quantitative yield in the form of an amorphous white solid which was employed without further chromatographic purification in subsequent reactions.

¹H-NMR (CHLOROFORM-d): δ [ppm]=7.12 (d, 1H); 7.79 (s, 1H); 7.90, (d, 1H).

### Intermediate IV-2

### 6-Chloro-3-(furo[3,2-c]pyridin-2-yl)imidazo[1,2-b]pyridazine

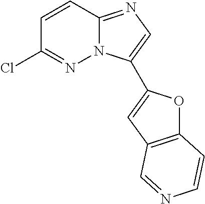 [SMILES: Clc1ccc2ncc(-c3cc4cnccc4o3)n2n1]

A mixture of 2.0 g (17 mmol) furo[3,2-c]-pyridine in anhydrous THF (98 mL) was cooled to −78° C. 16 mL (25 mmol) of a 1.6 M solution of n-butyllithium in hexane was added and the resulting mixture stirred for 1 h at −78° C. 7 mL (25 mmol) of tributyltin chloride were added at −78° C. The cooling bath was removed and the reaction was stirred at room temperature for 19 h.

Methanol was carefully added and the solvent evaporated. The obtained residue was purified by flash chromatography to yield 6.8 g of crude product of the corresponding 2-stannylfuro[3,2-c]pyridine, which was used without further purification.

In an inert atmosphere, 3 g (13 mmol) of 3-bromo-6-chloro-imidazo[1,2-b]pyridazine, 6.8 g (17 mmol) of the crude 2-stannylfuro[3,2-c]pyridine, 246 mg (1.3 mmol) copper (I) iodide and 452 mg (0.65 mmol) bis(triphenylphosphine) palladium(II)chloride in 130 mL of THF was stirred at reflux for 17 h.

The mixture was cooled to room temperature. The precipitate was filtered of and digested with a mixture of dichloromethane and hexane to give 1 g of the title compound as a crude product, which was used without further purification.

LCMS (Method 3): Rₜ=0.59 min; MS (ESIpos) m/z=271 [M+H]+.

### Intermediate IV-3

### 6-Chloro-3-(4-methoxyfuro[3,2-c]pyridin-2-yl)imidazo[1,2-b]pyridazine

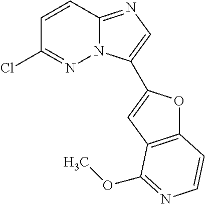 [SMILES: COc1nccc2oc(-c3cnc4ccc(Cl)nn34)cc12]

A mixture of 5.0 g (34 mmol) 4-methoxy-furo[3,2-c]-pyridine in anhydrous THF (230 mL) was cooled to −78° C. 20 mL (50 mmol) of a 2.5 M solution of n-butyllithium in hexane was added and the resulting mixture stirred for 1 h at −78° C. 13.5 mL (50 mmol) of tributyltin chloride were added at −78° C. The cooling bath was removed and the reaction was stirred at room temperature over night.

Methanol was carefully added and the solvent evaporated. The obtained residue was purified by flash chromatography to yield 15 g of crude product of the corresponding 2-stannylfuro[3,2-c]pyridine, which was used without further purification.

In an inert atmosphere, 6 g (26 mmol) of 3-bromo-6-chloro-imidazo[1,2-b]pyridazine, 15 g (33 mmol) of the crude 2-stannylfuro[3,2-c]pyridine, 491 mg (2.6 mmol) copper (I) iodide and 905 mg (1.3 mmol) bis(triphenylphosphine) palladium(II)chloride in 250 mL of THF was stirred at reflux for 36 h.

The mixture was cooled to room temperature and 1000 mL of dichloromethane were added. The precipitate was filtered of and digested with 40 mL of a 1:1 mixture of dichloromethane and methanol to give 6.2 of the title compound as a crude product, which was used without further purification.

LCMS (Method 3): Rₜ=1.22 min; MS (ESIpos) m/z=301 [M+H]⁺.

### Intermediate IV-4

### 6-Chloro-3-[4-(propan-2-yloxy)furo[3,2-c]pyridin-2-yl]imidazo[1,2-b]pyridazine

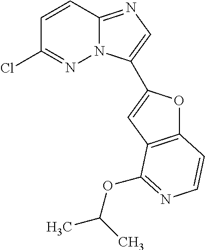 [SMILES: CC(C)Oc1nccc2oc(-c3cnc4ccc(Cl)nn34)cc12]

Step 1: At 0° C., 3.1 g (78 mmol) sodium hydride (60% suspension in mineral oil) was carefully added to 4.7 g (78 mmol) isopropanol in 100 mL of anhydrous THF. The mixture was stirred at 0° C. for 15 min. 3 g (19.5 mmol) 4-chlorofuro[3,2-c]pyridine was added. The mixture was stirred at 80° C. for 20 h.

Water was carefully added. The volume of the resulting suspension was reduced by evaporation. Water was added. The aqueous layer was extracted consecutively with ethyl acetate. The combined organic layers were washed with brine, dried over sodium sulfate and evaporated to give 4.6 g of a crude product, which was used without further purification in step 2.

Step 2: 3.5 g (19.5 mmol) of the crude product from step 1 in 44 mL anhydrous THF was cooled to −78° C. 11.7 mL (29 mmol) of a 2.5 M solution of n-butyl lithium in hexane was added. The mixture was stirred for 90 min at −78° C. 6.8 mL (29 mmol) of triisopropyl borate was added at −78° C. The cooling bath was removed and the mixture was stirred at room temperature for 1 h.

A small amount of water was added and the solvent was evaporated to 7.7 g of a crude product which was used without further purification in step 3.

Step 3: To 1.9 g (8 mmol) 3-bromo-6-chloroimidazo[1,2-b]pyridazine in 68 mL dioxane were added 1.9 g (8.4 mmol) of the crude product from step 2, 370 mg (0.32 mmol) tetrakis(triphenylphosphin)palladium(0) and 12 mL of a 2 M aqueous solution of sodium carbonate. The mixture was stirred at 100° C. for 18 h.

The reaction mixture was poured into saturated aqueous ammonium chloride solution and extracted with ethyl acetate. The organic layer was washed with brine, dried over sodium sulfate, and concentrated. The obtained solid material was digested with a 9:1 mixture of dichloromethane and methanol, filtered off, washed with dichloromathene and dried in vacuo to give 428 mg of the title compound as solid material. The mother liquor was concentrated and subjected to flash chromatography to give another fraction of product containing material, which was again digested in methanol and dichlormethane to give another 316 mg of the title compound.

¹H-NMR (400 MHz, DMSO-d₆), δ [ppm]=1.38 (6H), 5.47 (1H), 7.33 (1H), 7.44 (1H), 7.53 (1H), 8.03 (1H), 8.36-8.40 (2H).

LCMS (Method 3): Rₜ=1.43 min; MS (ESIpos) m/z=329 [M+H]⁺.

### Intermediate IV-5

### 6-Chloro-3-[4-(2,2-dimethyl propoxy)furo[3,2-c]pyridin-2-yl]imidazo[12-b]pyridazine

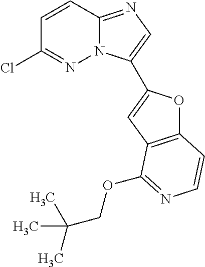 [SMILES: CC(C)(C)COc1nccc2oc(-c3cnc4ccc(Cl)nn34)cc12]

6-Chloro-3-[4-(2,2-dimethylpropoxy)furo[3,2-c]pyridin-2-yl]imidazo[1,2-b]pyridazine was prepared in analogy to 6-chloro-3-[4-(propan-2-yloxy)furo[3,2-c]pyridin-2-yl]imidazo[1,2-b]pyridazine starting from 2.8 g (12.2 mmol) of 3-bromo-6-chloro-imidazo[1,2-b]pyridazine to yield 1.3 g of the title compound after digestion in a 9:1 mixture of dichloromethane and methanol.

¹H-NMR (400 MHz, DMSO-d₆), δ [ppm]=1.03 (9H), 4.15 (2H), 7.35 (1H), 7.47 (1H), 7.53 (1H), 8.01 (1H), 8.37 (1H).

LCMS (Method 3): Rₜ=1.59 min; MS (ESIpos) m/z=357 [M+H]⁺.

### Intermediate IV-6

### 6-Chloro-3-[4-(cyclopropylmethoxy)furo[3,2-c]pyridin-2-yl]imidazo[1,2-b]pyridazine

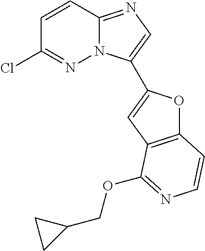 [SMILES: Clc1ccc2ncc(-c3cc4c(OCC5CC5)nccc4o3)n2n1]

6-Chloro-3-[4-(cyclopropylmethoxy)furo[3,2-c]pyridin-2-yl]imidazo[1,2-b]pyridazine was prepared in analogy to 6-chloro-3-[4-(propan-2-yloxy)furo[3,2-c]pyridin-2-yl]imidazo[1,2-b]pyridazine starting from 3.5 g (14.9 mmol) of 3-bromo-6-chloro-imidazo[1,2-b]pyridazine to yield 1.9 g of the title compound after digestion in methanol.

¹H-NMR (300 MHz, DMSO-d₆), δ [ppm]=0.37 (2H), 0.51-0.64 (2H), 1.33 (1H), 4.26 (2H), 7.33 (1H), 7.43 (1H), 7.52 (1H), 8.00 (1H), 8.32-8.41 (2H).

LCMS (Method 2): Rₜ=1.37 min; MS (ESIpos) m/z=341 [M+H]⁺.

### Intermediate IV-7

### N-Ethylfuro[3,2-c]pyridin-4-amine

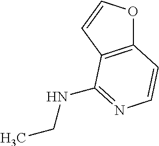

A stirred suspension of 4-chlorofuro[3,2-c]pyridine (1.5 g), ethylamine hydrochloride (2.39 g) and Hünig base (5.0 mL) in 2-propanol (7.5 mL) was heated to 130° C. in a microwave oven for 20 h. A half-saturated solution of sodium bicarbonate was added and the mixture was extracted with ethyl acetate. The organic phase was washed with saturated sodium chloride solution, dried (sodium sulfate) and the solvent was removed in vacuum. Silicagel chromatography gave 793 mg of the title compound.

¹H-NMR (300 MHz, DMSO-d₆), δ [ppm]=1.15 (3H), 3.40 (2H), 6.73 (1H), 6.87 (1H), 7.03 (1H), 7.75 (1H), 7.78 (1H).

LCMS (Method 5): Rₜ=0.86 min; MS (ESIpos) m/z=163 [M+H]⁺.

### Intermediate IV-8

### tert-Butyl ethyl(furo[3,2-c]pyridin-4-yl)carbamate

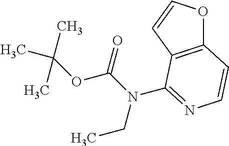 [SMILES: CCN(C(=O)OC(C)(C)C)c1nccc2occc12]

To a stirred solution of N-ethylfuro[3,2-c]pyridin-4-amine (940 mg) and Hünig base (3.0 mL) in THF (50 mL) was added di-tert-butyl dicarbonate (1.52 g) and the mixture was stirred at 65° C. for 24 h. A half-saturated solution of sodium bicarbonate was added and the mixture was extracted with ethyl acetate. The organic phase was washed with saturated sodium chloride solution, dried (sodium sulfate) and the solvent was removed in vacuum. Silicagel chromatography gave 1.38 g of the title compound.

¹H-NMR (400 MHz, DMSO-d₆), δ [ppm]=1.09 (3H), 1.35 (9H), 3.80 (2H), 6.74 (1H), 7.52 (1H), 8.04 (1H), 8.25 (1H).

LCMS (Method 5): Rₜ=1.20 min; MS (ESIpos) m/z=263 [M+H]⁺.

### Intermediate IV-9

### {4-[(tert-Butoxycarbonyl)(ethyl)amino]furo[3,2-c]pyridin-2-yl}boronic acid

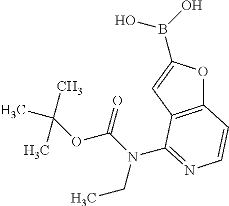 [SMILES: CCN(C(=O)OC(C)(C)C)c1nccc2oc(B(O)O)cc12]

To a stirred solution of tert-butyl ethyl(furo[3,2-c]pyridin-4-yl)carbamate (1.86 g) in anhydrous THF (20 mL) was added a solution of n-butyllithium in hexane (3.8 mL; c=2.5 M) at −78° C. The solution was stirred at −78° C. for 1.5 h. Triisopropyl borate (1.92 g) was added at −78° C., and the mixture was stirred at −78° C. for 0.5 h and allowed to warm up to room temperature within 16 h. Water was added, the reaction mixture was stirred for 15 minutes and the solvent was removed in vacuum. Again, water was added and the mixture was lyophilized to give 1.98 g of the title compound as a crude product which was used without purification.

LCMS (Method 5): Rₜ=0.46 min; MS (ESIpos) m/z=307 [M+H]⁺.

### Intermediate IV-10

### (5R)-5-{[(3-Bromoimidazo[1,2-b]pyridazin-6-yl)oxy]methyl}pyrrolidin-2-one

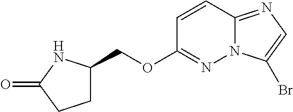 [SMILES: O=C1CC[C@H](COc2ccc3ncc(Br)n3n2)N1]

To a stirred suspension of (5R)-5-(hydroxymethyl)pyrrolidin-2-one (2.23 g) in anhydrous THF (40 mL) and anhydrous DMF (20 mL) was added sodium hydride (60% w/w in oil; 1.03 g) at 0° C. and the mixture was stirred at 0° C. for 30 minutes. 3-bromo-6-chloroimidazo[1,2-b]pyridazine (3.0 g) was added and the mixture was stirred at room temperature for 60 hours. Water was added and the mixture was extracted with a mixture of dichloromethane and methanol (100:1). The organic phase was dried (sodium sulfate) and the solvent was removed in vacuum. Silicagel chromatography gave a solid that was recrystallized from ethyl acetate to give 2.7 g of the title compound.

¹H-NMR (400 MHz, DMSO-d₆), δ [ppm]=1.81-1.92 (1H), 2.05-2.32 (3H), 3.90-4.01 (1H), 4.18-4.34 (2H), 6.92 (1H), 7.71 (1H), 7.84 (1H), 8.03 (1H).

### Intermediate IV-11

### tert-Butyl ethyl[2-(6-{[(2R)-5-oxopyrrolidin-2-yl]methoxy}imidazo[1,2-b]pyridazin-3-yl)furo[3,2-c]pyridin-4-yl]carbamate

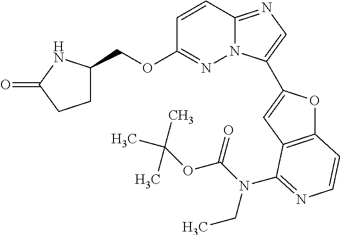 [SMILES: CCN(C(=O)OC(C)(C)C)c1nccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (130 mg) in 1-propanol (11 ml) was added 2M potassium carbonate solution (0.6 ml), crude {4-[(tert-butoxycarbonyl)(ethyl)amino]furo[3,2-c]pyridin-2-yl}boronic acid (70% w/w; 362 mg), triphenylphosphine (10.9 mg) and bis(triphenylphosphine)palladium(II) chloride (29.3 mg). The mixture was heated to reflux for 1 h. The warm mixture was filtered through Celite the solvent was removed in vacuum. A half-saturated solution of sodium bicarbonate was added and the mixture was extracted with a mixture of dichloromethane and methanol. The organic phase was washed with saturated sodium chloride solution, dried (sodium sulfate) and the solvent was removed in vacuum. Silicagel chromatography gave a solid that was triturated with a mixture of dichloromethane and hexane to give 135 mg of the title compound.

¹H-NMR (300 MHz, DMSO-d₆), δ [ppm]=1.16 (3H), 1.37 (9H), 1.86 (1H), 2.09-2.33 (3H), 3.83 (2H), 3.99 (1H), 4.22-4.32 (1H), 4.35-4.45 (1H), 7.07 (1H), 7.29 (1H), 7.60 (1H), 7.94 (1H), 8.17-8.24 (2H), 8.30 (1H).

LC-MS (Method 5): Rₜ=1.05 min; MS (ESIpos) m/z=493 [M+H]⁺.

### Intermediate IV-12

### 3-(Furo[3,2-c]pyridin-2-yl)-6-{2-[(2S)-pyrrolidin-2-yl]ethoxy}imidazo[1,2-b]-pyridazine

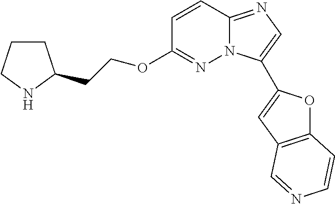 [SMILES: c1cc2oc(-c3cnc4ccc(OCC[C@@H]5CCCN5)nn34)cc2cn1]

Step 1: To 9.3 g (40.4 mmol) [(2S)-1-(tert-butoxycarbonyl)pyrrolidin-2-yl]acetic acid in 116 mL THF were added dropwise 40 mL of borane-dimethyl sulfide complex. The resulting mixture was stirred for 2 h at 80° C.

The mixture was carefully poured into saturated aqueous sodium hydrogencarbonate solution. The aqueous layer was extracted with methyl-tert-butylether. The combined organic layers were washed with brine, dried over magnesium sulfate, and concentrated to give 6.2 g of a crude product which was used without further purification in step 2.

Step 2: In an ice bath, 179 mg (0.83 mmol) of the crude product from step 1 were added to 44 mg (1.1 mmol) sodium hydride (60% in mineral oil) in 7 mL anhydrous THF. After 15 min of stirring in the ice bath, 150 mg (0.55 mmol) 6-chloro-3-(furo[2,3-c]pyridin-2-yl)imidazo[1,2-b]pyridazine were added. The ice bath was removed and the reaction mixture was stirred for 17 h at room temperature.

The reaction mixture was poured into water, and extracted with ethyl acetate. The combined organic phases were washed with brine, dried over magnesium sulfate, and concentrated. The obtained crude product (298 mg) was used without further purification in step 3.

Step 3: To 298 mg of the crude product from step 2 in 6 mL dichloromethane were added 1.2 mL of TFA. The mixture was stirred for 90 min. Aqueous ammonia solution was added until the mixture reached basic pH. Brine was added and the mixture was extracted with dichloromethane. The organic layer was separated, dried over magnesium sulfate and concentrated.

The obtained crude product (250 mg) was used without further purification.

90 mg of the crude product were purified by HPLC to give 13 mg of the product as solid material.

¹H-NMR (300 MHz, DMSO-d₆), δ [ppm]=1.48-1.67 (1H), 1.72-1.97 (2H), 2.23 (2H), 2.93-3.23 (2H), 3.45-3.62 (2H), 4.53-4.74 (2H), 6.99-7.17 (1H), 7.66-7.86 (2H), 8.12-8.28 (2H), 8.28-8.45 (1H), 8.45-8.60 (1H), 8.93-9.14 (1H).

LC-MS (Method 3): Rₜ=0.49 min; MS (ESIpos) m/z=350 [M+H]⁺.

### Intermediate IV-13

### 3-(4-Methoxyfuro[3,2-c]pyridin-2-yl)-6-[(2R)-morpholin-2-ylmethoxy]imidazo-[1,2-b]pyridazine

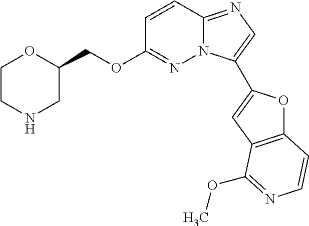 [SMILES: COc1nccc2oc(-c3cnc4ccc(OC[C@H]5CNCCO5)nn34)cc12]

1.75 g (15 mmol) (2R)-Morpholin-2-ylmethanol were dissolved in 50 mL anhydrous DMF. At 0−5° C. 600 mg (15 mmol) sodium hydride (60% in mineral oil) were added. After 10 min on the ice bath 1.5 g (4.04 mmol) 6-chloro-3-(4-methoxyfuro[3,2-c]pyridin-2-yl)imidazo[1,2-b]pyridazine were added. The ice bath was removed and it was stirred 24 h at room temperature. Then 80 mg (2.0 mmol) sodium hydride (60% in mineral oil) were added and 6 h later the solvent was removed.

Saturated ammonium chloride solution was added and it was extracted four times with dichloromethane. The combined organic phases were washed twice with water, dried over magnesium sulfate and concentrated. The residue was purified by silica gel (dichloromethane and methanol) to yield 930 mg (60%) material and 360 mg (22%) of slightly impure material, which was purified by HPLC to yield additional 207 mg (13%) of product.

¹H-NMR (300 MHz, DMSO-d₆), δ [ppm]=2.55-2.65 (1H), 2.65-2.73 (2H), 2.92-3.00 (1H), 3.44-3.55 (1H), 3.74-3.82 (1H), 3.83-3.92 (1H), 4.02 (3H), 4.35-4.46 (2H), 7.02-7.09 (1H), 7.33-7.38 (1H), 7.48 (1H), 8.00-8.06 (1H), 8.11-8.20 (2H).

LC-MS (Method 2): Rₜ=0.69 min; MS (ESIpos) m/z=382 [M+H]⁺.

### Intermediate IV-14

### (6R)-4-(2,2-dimethylpropanoyl)-6-(hydroxymethyl)piperazin-2-one

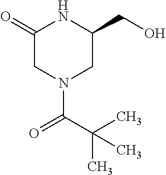

(6R)-4-(2,2-Dimethylpropanoyl)-6-(hydroxymethyl)piperazin-2-one was prepared as described in Organic Letters, 2004, Vol. 6, pages 4096-4072.

### EXAMPLES

### Example I-1

### [(2S)-2-(2-{[3-(1-Benzofuran-2-yl)imidazo[1,2-b]pyridazin-6-yl]oxy}ethyl)-pyrrolidin-1-yl](cyclopropyl)methanone

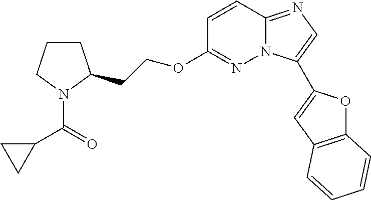 [SMILES: O=C(C1CC1)N1CCC[C@H]1CCOc1ccc2ncc(-c3cc4ccccc4o3)n2n1]

To 50 mg (0.14 mmol) 3-(1-benzofuran-2-yl)-6-{2-[(2S)-pyrrolidin-2-yl]ethoxy}imidazo[1,2-b]-pyridazine in 2 mL dichloromethane were added 23 μL (0.29 mmol) pyridine and 18 μL (0.17 mmol) cyclopropanecarbonyl chloride. The mixture was stirred for 24 h at room temperature. 80 μL (0.58 mmol) triethylamine were added and the mixture was stirred for another 72 h at room temperature.

50 μL of water were added and the mixture was concentrated. The obtained crude product was purified by HPLC to give 18 mg of the title compound as solid material.

¹H-NMR (400 MHz, DMSO-d₆), δ [ppm]=0.46-0.76 (4H), 1.70-2.08 (6H), 2.13-2.26 (1H), 3.56-3.71 (2H), 4.23 (1H), 4.36-4.56 (3H), 4.56-4.66 (1H), 6.94-7.05 (1H), 7.24-7.36 (2H), 7.59-7.68 (2H), 7.72 (1H), 8.11-8.18 (2H).

LC-MS (Method 1): Rₜ=1.26 min; MS (ESIpos) m/z=417 [M+H]⁺.

### Example I-2

### 1-[(2S)-2-(2-{[3-(1-Benzofuran-2-yl)imidazo[1,2-b]pyridazin-6-yl]oxy}ethyl)-pyrrolidin-1-yl]ethanone

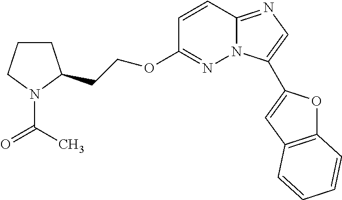 [SMILES: CC(=O)N1CCC[C@H]1CCOc1ccc2ncc(-c3cc4ccccc4o3)n2n1]

To 168 mg (0.2 mmol) 3-(1-benzofuran-2-yl)-6-{2-[(2S)-pyrrolidin-2-yl]ethoxy}imidazo[1,2-b]-pyridazine in 1 mL THF were added 74 μL (0.78 mmol) acetic anhydride and 63 μL (0.78 mmol) pyridine. The mixture was stirred for 3 h at room temperature.

Water was added. After 5 min of stirring, saturated aqueous sodium hydrogen carbonate solution was added. The mixture was extracted with ethyl acetate. The organic layer was dried over magnesium sulfate and evaporated. The obtained crude product was purified by flash chromatography to give 68 mg of the title compound as solid material.

¹H-NMR (400 MHz, DMSO-d₆, 78.5° C.): δ [ppm]=1.80-2.07 (10H), 3.47 (2H), 4.23 (1H), 4.58 (2H), 6.97 (1H), 7.26-7.37 (2H), 7.62 (1H), 7.65 (1H), 7.74 (1H), 8.09 (1H), 8.12 (1H).

LC-MS (Method 1): Rₜ=1.15 min; MS (ESIpos) m/z=391 [M+H]⁺.

### Example I-3

### 1-[(2S)-2-(2-{[3-(1-Benzofuran-2-yl)imidazo[1,2-b]pyridazin-6-yl]oxy}ethyl)-pyrrolidin-1-yl]-2,2-dimethylpropan-1-one

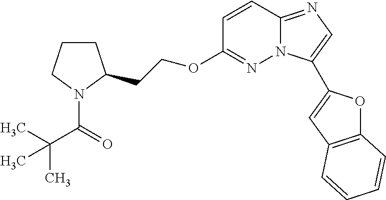 [SMILES: CC(C)(C)C(=O)N1CCC[C@H]1CCOc1ccc2ncc(-c3cc4ccccc4o3)n2n1]

To 250 mg (0.72 mmol) 3-(1-benzofuran-2-yl)-6-{2-[(2S)-pyrrolidin-2-yl]ethoxy}imidazo[1,2-b]-pyridazine in 10 mL tetrahydrofurane were added 582 μL (2.9 mmol) 2,2-dimethylpropanoic anhydride and 231 μL (2.9 mmol) pyridine. The mixture was stirred for 2 h at room temperature.

Water was added. After 5 min of stirring, saturated aqueous sodium hydrogen carbonate solution was added. The mixture was extracted with ethyl acetate. The organic layer was dried over magnesium sulfate and evaporated. The obtained crude product was digested in a 1:1 mixture of dichloromethane and methyl tert-butyl ether to give 144 mg of the title compound as solid material.

¹H-NMR (300 MHz, DMSO-d₆), δ [ppm]=1.09-1.18 (9H), 1.66-2.01 (5H), 2.09-2.27 (1H), 3.44-3.57 (1H), 3.60-3.72 (1H), 4.21-4.33 (1H), 4.37-4.56 (2H), 6.97 (1H), 7.29 (2H), 7.58-7.65 (2H), 7.71 (1H), 8.13 (2H).

LC-MS (Method 2): Rₜ=1.42 min; MS (ESIpos) m/z=433 [M+H]⁺.

### Example I-4

### 4-({[3-(1-Benzofuran-2-yl)imidazo[1,2-b]pyridazin-6-yl]oxy}methyl)-1,3-oxazolidin-2-one

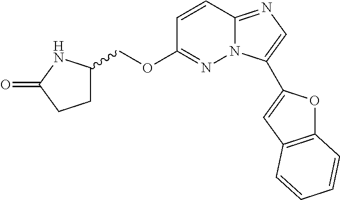 [SMILES: O=C1CCC(COc2ccc3ncc(-c4cc5ccccc5o4)n3n2)N1]

At 0-5° C. 130 mg (1.11 mmol) 4-(hydroxymethyl)-1,3-oxazolidin-2-one were added to 44.5 mg (1.11 mmol) sodium hydride (60% in mineral oil) in 7.5 mL anhydrous DMF. After 5 minutes of stirring on the ice bath, 150 mg (0.56 mmol) 3-(1-benzofur-2-yl)-6-chloroimidazo[1,2-b]pyridazine were added. The ice bath was removed and it was stirred 72 hours at room temperature. The reaction mixtures were poured into half saturated ammonium chloride solution, and extracted four times with ethyl acetate. The combined organic phases were washed with brine, dried over magnesium sulfate, and concentrated. The residue was purified by HPLC to yield 38 mg (20%) product.

¹H-NMR (400 MHz, DMSO-d₆), δ [ppm]=4.27-4.35 (2H), 4.44-4.56 (3H), 7.01 (1H), 7.25-7.36 (2H), 7.61-7.65 (2H), 7.72 (1H), 7.99 (1H), 8.15-8.19 (2H).

LC-MS (Method 2): Rₜ=1.00 min; MS (ESIpos) m/z=350 [M+H]⁺.

### Example I-5

### N-(trans-3-{[3-(1-Benzofuran-2-yl)imidazo[1,2-b]pyridazin-6-yl]oxy}cyclobutyl)-cyclopropanecarboxamide

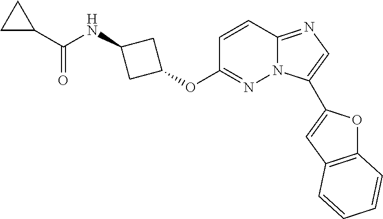 [SMILES: O=C(N[C@H]1C[C@H](Oc2ccc3ncc(-c4cc5ccccc5o4)n3n2)C1)C1CC1]

To 100 mg (0.31 mmol) trans-3-{[3-(1-benzofuran-2-yl)imidazo[1,2-b]pyridazin-6-yl]oxy}cyclo-butanamine in 5 mL THF were added 51 μL (0.62 mmol) pyridine and 43 μL (0.47 mmol) cyclopropanecarbonyl chloride. The mixture was stirred for 24 h at room temperature.

50 μL of water were added and the mixture was stirred for 5 min. Ammonia (25% in water) was added and the mixture was extracted with ethyl acetate. The combined organic layers were dried over sodium sulphate and evaporated. The obtained crude product was purified by HPLC to give 21 mg of the title compound as solid material.

¹H-NMR (300 MHz, DMSO-d₆), δ [ppm]=0.63-0.71 (4H), 1.50-1.61 (1H), 2.57 (4H), 4.31-4.46 (1H), 5.41-5.54 (1H), 7.04 (1H), 7.31 (2H), 7.57 (1H), 7.63 (1H), 7.68-7.74 (1H), 8.12-8.20 (2H), 8.56 (1H).

LC-MS (Method 3): Rₜ=1.14 min; MS (ESIpos) m/z=389 [M+H]⁺.

### Example I-6

### 1-[(2S)-2-(2-{[3-(1-Benzofuran-2-yl)imidazo[1,2-b]pyridazin-6-yl]oxy}ethyl)-pyrrolidin-1-yl]-3,3-dimethylbutan-1-one

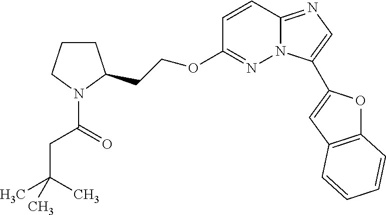 [SMILES: CC(C)(C)CC(=O)N1CCC[C@H]1CCOc1ccc2ncc(-c3cc4ccccc4o3)n2n1]

To 50 mg (0.14 mmol) 3-(1-benzofuran-2-yl)-6-{2-[(2S)-pyrrolidin-2-yl]ethoxy}imidazo[1,2-b]-pyridazine in 2 mL dichloromethane were added 23 μL (0.29 mmol) pyridine and 24 μL (0.17 mmol) 3,3-dimethylbutanoyl chloride. The mixture was stirred for 24 h at room temperature. 80 μL (0.58 mmol) triethylamine were added and the mixture was stirred for another 48 h at room temperature.

50 μL of water were added and the mixture was concentrated. The obtained crude product was purified by HPLC to give 18 mg of the title compound as solid material.

¹H-NMR (400 MHz, DMSO-d₆): δ [ppm]=0.97 (5H), 1.78-2.15 (6H), 3.49 (1H), 3.67 (1H), 4.22-4.44 (1H), 4.51-4.66 (2H), 6.96 (1H), 7.24-7.37 (2H), 7.61 (2H), 7.70-7.75 (1H), 8.05-8.16 (2H).

LC-MS (Method 1): Rₜ=1.44 min; MS (ESIpos) m/z=447 [M+H]⁺.

### Example I-7

### (5S)-5-({[3-(1-Benzofuran-2-yl)imidazo[1,2-b]pyridazin-6-yl]oxy}methyl)-pyrrolidin-2-one

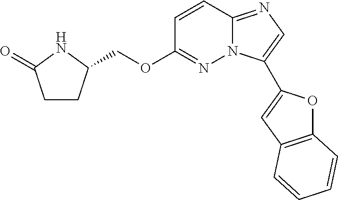 [SMILES: O=C1CC[C@@H](COc2ccc3ncc(-c4cc5ccccc5o4)n3n2)N1]

In an ice bath, 174 mg (1.5 mmol) (S)-5-(hydroxymethyl)-2-pyrrolidinone were added to 52 mg (1.3 mmol) sodium hydride (60% in mineral oil) in 25 mL anhydrous THF. After 15 min of stirring on the ice bath, 200 mg (0.74 mmol) of 3-(1-benzofur-2-yl)-6-chloroimidazo[1,2-b]pyridazine were added. The ice bath was removed and the mixture was stirred for 16 h at room temperature. 147 mg (0.74 mmol) potassium 1,1,1,3,3,3-hexamethyldisilazan-2-ide were added. Stirring at room temperature was continued for 72 h.

The reaction mixture was poured into saturated aqueous ammonium chloride solution and extracted with ethyl acetate. The organic layer was dried over magnesium sulfate, and concentrated. The residue was purified by flash chromatography to yield 145 mg of the title compound as solid material.

¹H-NMR (600 MHz, DMSO-d₆): δ [ppm]=2.01 (1H), 2.16-2.22 (1H), 2.23-2.30 (1H), 31-2.38 (1H), 4.05-4.11 (1H), 4.45-4.53 (2H), 7.06 (1H), 7.29-7.33 (1H), 7.36 (1H), 7.64 (1H), 7.66 (1H), 7.76 (1H), 7.95 (1H), 8.17-8.22 (2H).

LC-MS (Method 5): Rₜ=1.02 min; MS (ESIpos) m/z=349 [M+H]⁺.

### Example I-8

### 6-({[3-(1-Benzofuran-2-yl)imidazo[1,2-b]pyridazin-6-yl]oxy}methyl)piperidin-2-one

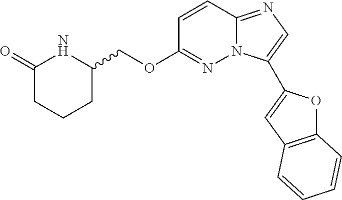 [SMILES: O=C1CCCC(COc2ccc3ncc(-c4cc5ccccc5o4)n3n2)N1]

In an ice bath, 67 mg (0.52 mmol) 6-(hydroxymethyl)piperidin-2-one were added to 18 mg (0.46 mmol) sodium hydride (60% in mineral oil) in 4 mL anhydrous THF. After 15 min of stirring on the ice bath, 70 mg (0.26 mmol) of 3-(1-benzofur-2-yl)-6-chloroimidazo[1,2-b]pyridazine were added. The ice bath was removed and the mixture was stirred for 15 h at 40° C.

The reaction mixture was poured into water and extracted with ethyl acetate. The organic layer was dried over magnesium sulfate, and concentrated. The residue was digested in methyl tert-butylether to yield 54 mg of the title compound as solid material.

¹H-NMR (400 MHz, DMSO-d₆), δ [ppm]=1.62-1.75 (2H), 1.83-2.02 (2H), 2.19 (2H), 3.84 (1H), 4.40-4.48 (1H), 4.50-4.57 (1H), 7.03-7.09 (1H), 7.27-7.39 (2H), 7.62-7.69 (3H), 7.74 (1H), 8.15-8.22 (2H).

LC-MS (Method 3): Rₜ=1.04 min; MS (ESIpos) m/z=363 [M+H]⁺.

### Example I-9

### (5R)-5-({[3-(1-Benzofuran-2-yl)imidazo[1,2-b]pyridazin-6-yl]oxy}methyl)-pyrrolidin-2-one

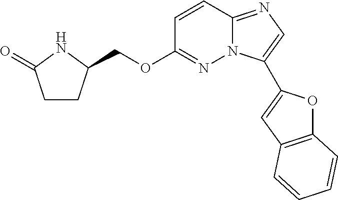 [SMILES: O=C1CC[C@H](COc2ccc3ncc(-c4cc5ccccc5o4)n3n2)N1]

In an ice bath, 174 mg (1.5 mmol) (R)-5-(hydroxymethyl)-2-pyrrolidinone in 2 mL DMF were added to 59 mg (1.5 mmol) sodium hydride (60% in mineral oil) in 6 mL anhydrous THF. After 15 min of stirring on the ice bath, 200 mg (0.74 mmol) of 3-(1-benzofur-2-yl)-6-chloroimidazo[1,2-b]pyridazine were added. The ice bath was removed and the mixture was stirred for 20 h at room temperature.

The reaction mixture was poured into water and extracted with ethyl acetate. The organic layer was dried over magnesium sulfate, and concentrated. The residue was purified by HPLC to yield 175 mg of the title compound as solid material.

¹H-NMR (400 MHz, DMSO-d₆), δ [ppm]=1.91-2.03 (1H), 2.10-2.36 (3H), 4.00-4.08 (1H), 4.40-4.50 (2H), 7.02 (1H), 7.24-7.36 (2H), 7.58-7.65 (2H), 7.73 (1H), 7.91 (1H), 8.12-8.19 (1H).

LC-MS (Method 3): Rₜ=1.0 min; MS (ESIpos) m/z=349 [M+H]⁺.

### Example I-10

Methyl (2S)-2-(2-{[3-(1-benzofuran-2-yl)imidazo[1,2-b]pyridazin-6-yl]oxy}-ethyl)pyrrolidine-1-carboxylate

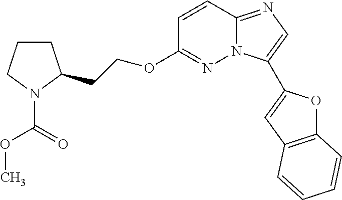 [SMILES: COC(=O)N1CCC[C@H]1CCOc1ccc2ncc(-c3cc4ccccc4o3)n2n1]

To 50 mg (0.14 mmol) 3-(1-benzofuran-2-yl)-6-{2-[(2S)-pyrrolidin-2-yl]ethoxy}imidazo[1,2-b]-pyridazine in 6 mL THF were added 100 μL (0.57 mmol) N-ethyl-N-(propan-2-yl)propan-2-amine and 45 μL (0.57 mmol) methyl carbonochloridoate. The mixture was stirred for 6 h at room temperature.

The mixture was concentrated. The obtained crude product was purified by HPLC to give 38 mg of the title compound as solid material.

1H-NMR (300 MHz, DMSO-d₆), δ [ppm]=1.75-2.02 (5H), 3.39-3.60 (3H), 4.02 (1H), 4.52 (2H), 6.99 (1H), 7.22-7.37 (2H), 7.54-7.68 (2H), 7.73 (1H), 8.11-8.18 (2H).

LC-MS (Method 3): Rₜ=1.32 min; MS (ESIpos) m/z=407 [M+H]⁺.

### Example I-11

### N-(trans-3-{[3-(1-Benzofuran-2-yl)imidazo[1,2-b]pyridazin-6-yl]oxy}cyclobutyl)-acetamide

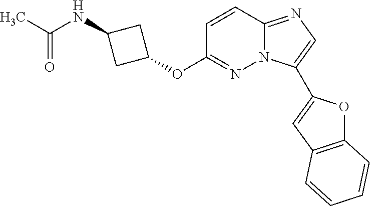 [SMILES: CC(=O)N[C@H]1C[C@H](Oc2ccc3ncc(-c4cc5ccccc5o4)n3n2)C1]

To 100 mg (0.31 mmol) trans-3-{[3-(1-benzofuran-2-yl)imidazo[1,2-b]pyridazin-6-yl]oxy}cyclo-butanamine in 5 mL THF were added 100 μL (0.12 mmol) pyridine and 118 μL (1.2 mmol) acetic anhydride. The mixture was stirred for 3 h at room temperature.

50 μL of water were added and the mixture was stirred for 5 min. Ammonia (25% in water) was added and the mixture was extracted with ethyl acetate. The combined organic layers were dried over sodium sulfate and evaporated. The obtained crude product was purified by HPLC to give 20 mg of the title compound as solid material.

¹H-NMR (400 MHz, DMSO-d₆), δ [ppm]=1.83-1.91 (3H), 2.54-2.61 (4H), 4.39 (1H), 5.49 (1H), 7.06 (1H), 7.28-7.40 (2H), 7.59 (1H), 7.66 (1H), 7.70-7.77 (1H), 8.14-8.22 (2H), 8.37 (1H).

LC-MS (Method 3): Rₜ=1.03 min; MS (ESIpos) m/z=363 [M+H]⁺.

### Example I-12

### 1-(2-{[3-(4-methoxy-1-benzofuran-2-yl)imidazo[1,2-b]pyridazin-6-yl]oxy}ethyl)-imidazolidin-2-one

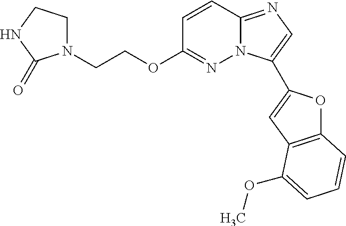 [SMILES: COc1cccc2oc(-c3cnc4ccc(OCCN5CCNC5=O)nn34)cc12]

In an ice bath, 62 mg (0.47 mmol) 1-(2-hydroxyethyl)imidazolidin-2-one were added to 16 mg (0.41 mmol) sodium hydride (60% in mineral oil) in 1.6 mL anhydrous THF. After 15 min of stirring on the ice bath, 70 mg (0.23 mmol) of 6-chloro-3-(4-methoxy-1-benzofuran-2-yl)imidazo[1,2-b]pyridazine were added. The ice bath was removed and the mixture was stirred for 96 h at room temperature.

The reaction mixture was poured into saturated aqueous ammonium chloride solution and extracted with ethyl acetate. The organic layer was dried over magnesium sulfate, and concentrated. The residue was digested in a mixture of dichloromethane and methyl tert-butyl ether to yield 37 mg of the title compound as solid material.

¹H-NMR (300 MHz, DMSO-d₆), δ [ppm]=3.18-3.25 (2H), 3.44-3.60 (4H), 4.55 (2H), 6.39 (1H), 6.83 (1H), 7.01 (1H), 7.19-7.32 (2H), 7.50 (1H), 8.08-8.19 (2H).

LCMS (Method 2): Rₜ=0.98 min; MS (ESIpos) m/z=394 [M+H]⁺.

### Example I-13

### (5S)-5-({[3-(5-methoxy-1-benzofuran-2-yl)imidazo[1,2-b]pyridazin-6-yl]oxy}methyl)pyrrolidin-2-one

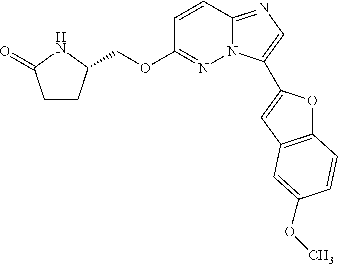 [SMILES: COc1ccc2oc(-c3cnc4ccc(OC[C@@H]5CCC(=O)N5)nn34)cc2c1]

In an ice bath, 55 mg (0.47 mmol) (S)-5-(hydroxymethyl)-2-pyrrolidinone were added to 16 mg (0.41 mmol) sodium hydride (60% in mineral oil) in 4 mL anhydrous THF. After 15 min of stirring on the ice bath, 70 mg (0.23 mmol) of 6-chloro-3-(5-methoxy-1-benzofuran-2-yl)imidazo[1,2-b]pyridazine were added. The ice bath was removed and the mixture was stirred for 16 h at 40° C. 47 mg (0.23 mmol) potassium 1,1,1,3,3,3-hexamethyldisilazan-2-ide were added. Stirring was continued for 96 h. Again, 47 mg (0.23 mmol) potassium 1,1,1,3,3,3-hexamethyldisilazan-2-ide were added. Stirring was continued for another 96 h.

The reaction mixture was poured into saturated aqueous ammonium chloride solution and extracted with ethyl acetate. The organic layer was dried over magnesium sulfate, and concentrated. The residue was purified by flash chromatography to yield 53 mg of the title compound as solid material.

¹H-NMR (400 MHz, DMSO-d₆), δ [ppm]=1.95-2.06 (1H), 2.14-2.38 (3H), 3.81 (3H), 4.01-4.10 (1H), 4.41-4.51 (2H), 6.92 (1H), 7.04 (1H), 7.25 (1H), 7.54 (1H), 7.57 (1H), 7.94 (1H), 8.14 (1H), 8.17 (1H).

LC-MS (Method 3): Rₜ=0.99 min; MS (ESIpos) m/z=378 [M+H]⁺.

### Example I-14

### 1-[2-({[3-(1-Benzofuran-2-yl)imidazo[1,2-b]pyridazin-6-yl]oxy}methyl)-morpholin-4-yl]-3,3-dimethylbutan-1-one

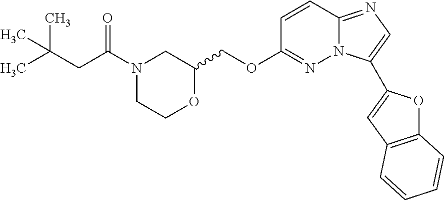 [SMILES: CC(C)(C)CC(=O)N1CCOC(COc2ccc3ncc(-c4cc5ccccc5o4)n3n2)C1]

To 80 mg (0.23 mmol) 3-(1-benzofuran-2-yl)-6-(morpholin-2-ylmethoxy)-imidazo[1,2-b]pyridazine in 2 mL dichloromethane were added 37 μL (0.46 mmol) pyridine and 38 μL (0.27 mmol) 3,3-dimethylbutanoyl chloride. The mixture was stirred for 24 h at room temperature.

50 μL of water were added and the mixture was concentrated. The obtained crude product was purified by HPLC to give 50 mg of the title compound as solid material.

¹H-NMR (400 MHz, DMSO-d₆, 80° C.): δ [ppm]=0.99 (9H), 2.25 (2H), 3.45-3.54 (1H), 3.85-3.97 (2H), 4.60 (2H), 7.03 (1H), 7.26-7.31 (1H), 7.34 (1H), 7.61 (2H), 7.75 (1H), 8.10-8.14 (2H).

LC-MS (Method 1): Rₜ=1.30 min; MS (ESIpos) m/z=449 [M+H]⁺.

### Example I-15

### N-(2-{[3-(1-Benzofuran-2-yl)imidazo[1,2-b]pyridazin-6-yl]oxy}ethyl)-cyclopropanecarboxamide

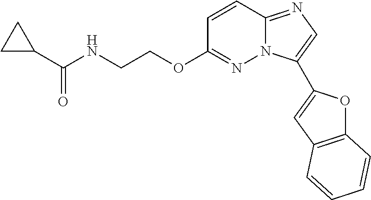 [SMILES: O=C(NCCOc1ccc2ncc(-c3cc4ccccc4o3)n2n1)C1CC1]

To 100 mg (0.34 mmol) 2-{[3-(1-benzofuran-2-yl)imidazo[1,2-b]pyridazin-6-yl]oxy}-ethanamine in 4 mL dichloromethane were added 55 μL (0.68 mmol) pyridine and 37 μL (0.41 mmol) cyclopropanecarbonyl chloride. The mixture was stirred for 24 h at room temperature.

50 μL of water were added and the mixture was concentrated. The obtained crude product was purified by HPLC to give 30 mg of the title compound as solid material.

¹H-NMR (300 MHz, DMSO-d₆), δ [ppm]=0.56-0.70 (4H), 1.50-1.61 (1H), 3.60 (2H), 4.53 (2H), 7.02 (1H), 7.30 (2H), 7.60-7.68 (2H), 7.72 (1H), 8.12-8.20 (2H), 8.32-8.42 (1H).

LCMS (Method 1): Rₜ=1.07 min; MS (ESIpos) m/z=363 [M+H]⁺.

### Example I-16

### [2-({[3-(1-benzofuran-2-yl)imidazo[1,2-b]pyridazin-6-yl]oxy}methyl)morpholin-4-yl](phenyl)methanone

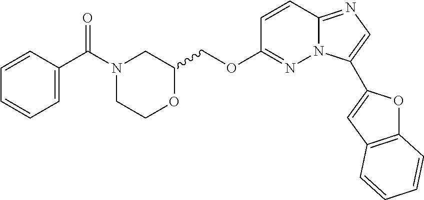 [SMILES: O=C(c1ccccc1)N1CCOC(COc2ccc3ncc(-c4cc5ccccc5o4)n3n2)C1]

To 80 mg (0.23 mmol) 3-(1-benzofuran-2-yl)-6-(morpholin-2-ylmethoxy)-imidazo[1,2-b]pyridazine in 2 mL dichloromethane were added 37 μL (0.46 mmol) pyridine and 32 μL (0.27 mmol) benzoyl chloride. The mixture was stirred for 24 h at room temperature.

50 μL of water were added and the mixture was concentrated. The obtained crude product was purified by HPLC to give 30 mg of the title compound as solid material.

¹H-NMR (400 MHz, DMSO-d₆, 80° C.): δ [ppm]=3.12-3.26 (2H), 3.60 (1H), 3.83 (1H), 3.94 (1H), 4.02 (1H), 4.18 (1H), 4.54-4.65 (2H), 6.99 (1H), 7.26-7.30 (1H), 7.34 (1H), 7.40 (5H), 7.58-7.64 (2H), 7.72 (1H), 8.11 (1H), 8.13 (1H).

LC-MS (Method 1): Rₜ=1.22 min; MS (ESIpos) m/z=455 [M+H]⁺.

### Example I-17

### N-(2-{[3-(1-Benzofuran-2-yl)imidazo[1,2-b]pyridazin-6-yl]oxy}ethyl)-3,3-dimethylbutanamide

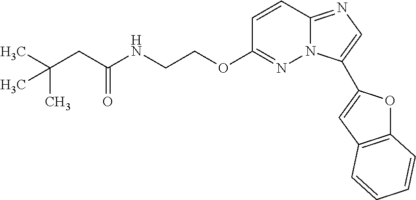 [SMILES: CC(C)(C)CC(=O)NCCOc1ccc2ncc(-c3cc4ccccc4o3)n2n1]

To 100 mg (0.34 mmol) 2-{[3-(1-benzofuran-2-yl)imidazo[1,2-b]pyridazin-6-yl]oxy}-ethanamine in 4 mL dichloromethane were added 55 μL (0.68 mmol) pyridine and 57 μL (0.41 mmol) 3,3-dimethylbutanoyl chloride. The mixture was stirred for 24 h at room temperature.

50 μL of water were added and the mixture was concentrated. The obtained crude product was purified by HPLC to give 35 mg of the title compound as solid material.

¹H-NMR (300 MHz, DMSO-d₆), δ [ppm]=0.92 (9H), 1.96 (2H), 3.57 (2H), 4.51 (2H), 6.97 (1H), 7.30 (2H), 7.59-7.67 (2H), 7.69-7.75 (1H), 8.02 (1H), 8.13-8.19 (2H).

LC-MS (Method 1): Rₜ=1.22 min; MS (ESIpos) m/z=393 [M+H]⁺.

### Example I-18

### 1-(2-{[3-(1-Benzofuran-2-yl)imidazo[1,2-b]pyridazin-6-yl]oxy}ethyl)-imidazolidin-2-one

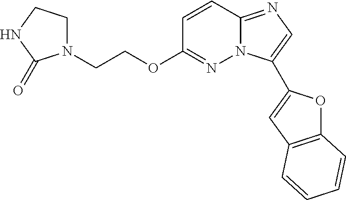 [SMILES: O=C1NCCN1CCOc1ccc2ncc(-c3cc4ccccc4o3)n2n1]

In an ice bath, 71 mg (0.52 mmol) 1-(2-hydroxyethyl)imidazolidin-2-one were added to 18 mg (0.46 mmol) sodium hydride (60% in mineral oil) in 2 mL anhydrous THF. After 15 min of stirring on the ice bath, 70 mg (0.26 mmol) of 6-chloro-3-(1-benzofuran-2-yl)imidazo[1,2-b]pyridazine were added. The ice bath was removed and the mixture was stirred for 72 h at room temperature.

The reaction mixture was poured into saturated aqueous ammonium chloride solution and extracted with ethyl acetate. The organic layer was dried over magnesium sulfate, and concentrated. The residue was digested in a mixture of methanol and methyl tert-butyl ether, filtered off, and digested a second time in methanol to yield 43 mg of the title compound as solid material.

¹H-NMR (300 MHz, DMSO-d₆), δ [ppm]=3.16-3.25 (2H), 3.44-3.52 (2H), 3.57 (2H), 4.58 (2H), 6.38 (1H), 7.01 (1H), 7.29 (2H), 7.59-7.68 (2H), 7.71-7.77 (1H), 8.12-8.18 (2H).

LC-MS (Method 1): Rₜ=0.99 min; MS (ESIpos) m/z=364 [M+H]⁺.

### Example I-19

### (5S)-5-({[3-(4-Methoxy-1-benzofuran-2-yl)imidazo[1,2-b]pyridazin-6-yl]oxy}-methyl)pyrrolidin-2-one

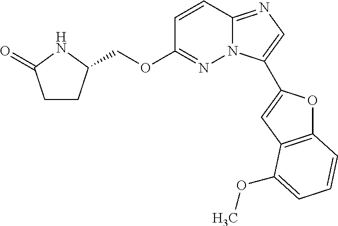 [SMILES: COc1cccc2oc(-c3cnc4ccc(OC[C@@H]5CCC(=O)N5)nn34)cc12]

In an ice bath, 55 mg (0.47 mmol) (S)-5-(hydroxymethyl)-2-pyrrolidinone were added to 16 mg (0.41 mmol) sodium hydride (60% in mineral oil) in 4 mL anhydrous THF. After 15 min of stirring on the ice bath, 70 mg (0.23 mmol) of 6-chloro-3-(4-methoxy-1-benzofuran-2-yl)imidazo[1,2-b]pyridazine were added. The ice bath was removed and the mixture was stirred for 16 h at 40° C. 47 mg (0.23 mmol) potassium 1,1,1,3,3,3-hexamethyldisilazan-2-ide were added. Stirring was continued for 96 h. Again, 47 mg (0.23 mmol) potassium 1,1,1,3,3,3-hexamethyldisilazan-2-ide were added. Stirring was continued for another 96 h.

The reaction mixture was poured into water and extracted with ethyl acetate. The organic layer was dried over magnesium sulfate, and concentrated. The residue was purified by flash chromatography to yield 58 mg of the title compound as solid material.

¹H-NMR (400 MHz, DMSO-d₆), δ [ppm]=1.91-2.00 (1H), 2.12-2.40 (3H), 3.94 (3H), 4.05-4.13 (1H), 4.39 (1H), 4.51 (1H), 6.85 (1H), 7.04 (1H), 7.22-7.34 (2H), 7.52 (1H), 7.90 (1H), 8.13-8.20 (2H).

LC-MS (Method 3): Rₜ=1.00 min; MS (ESIpos) m/z=379 [M+H]⁺.

### Example I-20

### 2,2,2-Trifluoro-1-[(2R)-2-({[3-(4-methoxy-1-benzofuran-2-yl)imidazo[1,2-b]-pyridazin-6-yl]oxy}methyl)morpholin-4-yl]ethanone

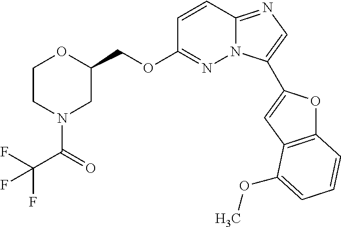 [SMILES: COc1cccc2oc(-c3cnc4ccc(OC[C@H]5CN(C(=O)C(F)(F)F)CCO5)nn34)cc12]

To 200 mg (0.53 mmol) 3-(4-methoxy-1-benzofuran-2-yl)-6-[(2R)-morpholin-2-ylmethoxy]imidazo[1,2-b]pyridazine in 6 mL dichloromethane were added 170 μL (2.1 mmol) pyridine and 146 μL (1.1 mmol) trifluoroacetic anhydride. The mixture was stirred for 24 h at 30° C.

The mixture was poured into brine and the mixture was extracted with dichloromethane. The combined organic layers were dried over sodium sulfate and evaporated. The obtained crude product was purified by HPLC to give 83 mg of the title compound as solid material.

¹H-NMR (400 MHz, DMSO-d₆), δ [ppm]=3.07-3.21 (1H), 3.42-3.56 (1H), 3.59-3.69 (1H), 3.79 (0.5H; likely, equatorial positions on morpholine ring), 3.92-4.16 (6H), 4.41 (0.5H; likely, equatorial positions on morpholine ring), 4.54-4.66 (2H), 6.86 (1H), 7.08 (1H), 7.24-7.33 (2H), 7.56 (1H), 8.14-8.21 (2H).

LC-MS (Method 3): Rₜ=1.29 min; MS (ESIpos) m/z=476 [M+H]⁺.

### Example I-21

### 1-[(2R)-2-({[3-(4-Methoxy-1-benzofuran-2-yl)imidazo[1,2-b]pyridazin-6-yl]-oxy}methyl)morpholin-4-yl]-2,2-dimethylpropan-1-one

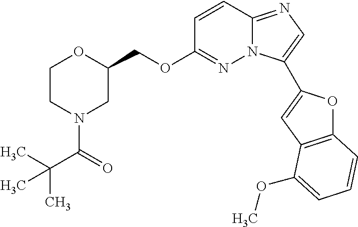 [SMILES: COc1cccc2oc(-c3cnc4ccc(OC[C@H]5CN(C(=O)C(C)(C)C)CCO5)nn34)cc12]

To 200 mg (0.53 mmol) 3-(4-methoxy-1-benzofuran-2-yl)-6-[(2R)-morpholin-2-ylmethoxy]imidazo[1,2-b]pyridazine in 7 mL dichloromethane were added 85 μL (1.1 mmol) pyridine and 79 μL (0.6 mmol) 2,2-dimethylpropanoyl chloride. The mixture was stirred for 24 h at 30° C.

The mixture was poured into brine and the mixture was extracted with dichloromethane. The combined organic layers were dried over sodium sulfate and evaporated. The obtained crude product was purified by HPLC to give 72 mg of the title compound as solid material.

¹H-NMR (400 MHz, DMSO-d₆), δ [ppm]=1.19 (9H), 3.02 (2H), 3.51 (1H), 3.83-3.90 (1H), 3.90-3.97 (4H), 4.15 (1H), 4.37 (1H), 4.57 (2H), 6.86 (1H), 7.08 (1H), 7.23-7.34 (2H), 7.57 (1H), 8.14-8.21 (2H).

LC-MS (Method 3): Rₜ=1.28 min; MS (ESIpos) m/z=465 [M+H]⁺.

### Example I-22

### 1-(3-{[3-(1-Benzofuran-2-yl)imidazo[1,2-b]pyridazin-6-yl]oxy}propyl)pyrrolidin-2-one

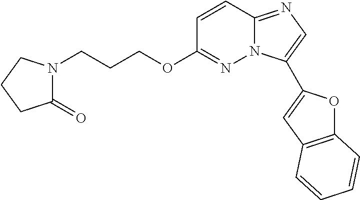 [SMILES: O=C1CCCN1CCCOc1ccc2ncc(-c3cc4ccccc4o3)n2n1]

In an ice bath, 78 mg (0.52 mmol) 1-(3-hydroxypropyl)pyrrolidin-2-one were added to 18 mg (0.46 mmol) sodium hydride (60% in mineral oil) in 2 mL anhydrous THF. After 15 min of stirring on the ice bath, 70 mg (0.23 mmol) of 6-chloro-3-(1-benzofuran-2-yl)imidazo[1,2-b]pyridazine were added. The ice bath was removed and the mixture was stirred for 16 h at room temperature.

The reaction mixture was poured into saturated aqueous ammonium chloride solution and extracted with ethyl acetate. The organic layer was dried over magnesium sulfate, and concentrated. The residue was digested in a mixture of dichloromethane and methyl tert-butyl ether to yield 58 mg of the title compound as solid material.

¹H-NMR (300 MHz, DMSO-d₆), δ [ppm]=1.89 (2H), 2.04 (2H), 2.13-2.24 (2H), 3.39 (4H), 4.47 (2H), 7.01 (1H), 7.22-7.37 (2H), 7.57-7.65 (2H), 7.74 (1H), 8.10-8.18 (2H).

LC-MS (Method 1): Rₜ=1.10 min; MS (ESIpos) m/z=377 [M+H]⁺.

### Example I-23

### N-(trans-3-{[3-(1-Benzofuran-2-yl)imidazo[1,2-b]pyridazin-6-yl]oxy}cyclobutyl)-2,2,2-trifluoroacetamide

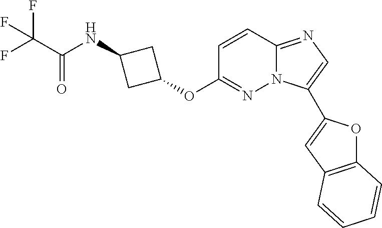 [SMILES: O=C(N[C@H]1C[C@H](Oc2ccc3ncc(-c4cc5ccccc5o4)n3n2)C1)C(F)(F)F]

To 100 mg (0.31 mmol) trans-3-{[3-(1-benzofuran-2-yl)imidazo[1,2-b]pyridazin-6-yl]oxy}cyclo-butanamine in 5 mL THF were added 101 μL (1.2 mmol) pyridine and 176 μL (1.2 mmol) trifluoroacetic anhydride. The mixture was stirred for 2 h at room temperature.

50 μL of water were added and the mixture was stirred for 5 min. Saturated aqueous sodium hydrogen carbonate solution was added and the mixture was extracted with ethyl acetate. The combined organic layers were dried over sodium sulfate and evaporated. The obtained crude product was purified by HPLC to give 48 mg of the title compound as solid material.

¹H-NMR (300 MHz, DMSO-d₆), δ [ppm]=2.59-2.81 (4H), 4.47 (1H), 5.47-5.59 (1H), 7.01-7.08 (1H), 7.24-7.37 (2H), 7.56 (1H), 7.60-7.69 (2H), 8.12-8.20 (2H).

LC-MS (Method 3): Rₜ=1.27 min; MS (ESIpos) m/z=417 [M+H]⁺.

### Example I-24

### 2-{[3-(1-Benzofuran-2-yl)imidazo[1,2-b]pyridazin-6-yl]oxy}acetamide

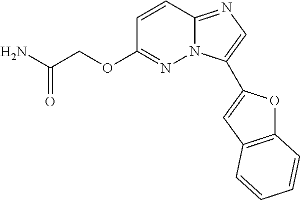 [SMILES: NC(=O)COc1ccc2ncc(-c3cc4ccccc4o3)n2n1]

35 mg (0.11 mmol) {[3-(1-benzofuran-2-yl)imidazo[1,2-b]pyridazin-6-yl]oxy}acetic acid in 3.5 mL DMF were treated with 24 μL (0.14 mmol) N-ethyl-N-isopropylpropan-2-amine, 52 mg (0.14 mmol) HATU and 250 μL (0.12 mmol) ammonia in THF (0.5 M solution). The mixture was stirred for 24 h at room temperature.

The solvent was evaporated and the residue was purified by HPLC to yield 2 mg of the title compound.

¹H-NMR (300 MHz, DMSO-d₆), δ [ppm]=4.89 (2H), 7.11 (1H), 7.24-7.38 (2H), 7.44 (1H), 7.59-7.71 (3H), 7.83 (1H), 8.15-8.23 (2H).

LC-MS (Method 5): Rₜ=0.96 min; MS (ESIpos) m/z=310 [M+H]⁺.

### Example I-25

### 2-{[3-(1-Benzofuran-2-yl)imidazo[1,2-b]pyridazin-6-yl]oxy}propanamide

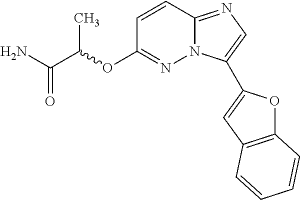 [SMILES: CC(Oc1ccc2ncc(-c3cc4ccccc4o3)n2n1)C(N)=O]

In an ice bath, 67 mg (0.74 mmol) 2-hydroxypropanamide were added to 26 mg (0.65 mmol) sodium hydride (60% in mineral oil) in 5 mL anhydrous THF. After 15 min of stirring on the ice bath, 100 mg (0.37 mmol) of 6-chloro-3-(1-benzofuran-2-yl)imidazo[1,2-b]pyridazine were added. The ice bath was removed and the mixture was stirred for 96 h at room temperature.

The reaction mixture was poured into saturated aqueous ammonium chloride solution and extracted with ethyl acetate. The organic layer was dried over magnesium sulfate, and concentrated. The residue purified by HPLC to yield 7 mg of the title compound as solid material.

¹H-NMR (300 MHz, DMSO-d₆), δ [ppm]=1.36 (3H), 4.24-4.36 (1H), 5.88 (1H), 7.23-7.37 (2H), 7.60-7.74 (2H), 7.96-8.06 (2H), 8.21-8.30 (2H).

LC-MS (Method 2): Rₜ=1.00 min; MS (ESIpos) m/z=323 [M+H]⁺.

### Example I-26

### 5-(2-{[3-(1-Benzofuran-2-yl)imidazo[1,2-b]pyridazin-6-yl]oxy}ethyl)pyrrolidin-2-one

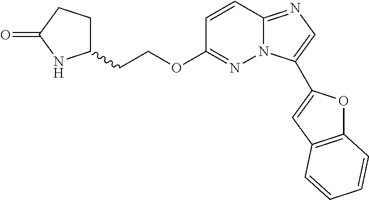 [SMILES: O=C1CCC(CCOc2ccc3ncc(-c4cc5ccccc5o4)n3n2)N1]

At 0-5° C. 144 mg (1.11 mmol) 5-(2-hydroxyethyl)pyrrolidin-2-one were added to 44.5 mg (1.11 mmol) sodiumhydride (60% in mineral oil) in 5.67 mL anhydrous DMF. After 5 minutes of stirring on the ice bath, 150 mg (0.56 mmol) 3-(1-benzofur-2-yl)-6-chloroimidazo[1,2-b]pyridazine were added. The ice bath was removed and it was stirred 1.5 h at room temperature.

The reaction mixture was poured into half saturated ammonium chloride solution. 20 mL ethyl acetate were added, the layers were separated and the aqueous phase was extracted twice with ethyl acetate. The combined organic phases were washed with brine, dried over magnesium sulfate, and concentrated. The residue was purified by HPLC affording 30 mg (15%) product.

¹H-NMR (400 MHz, DMSO-d₆), δ [ppm]=1.71-1.81 (1H), 1.97-2.09 (2H), 2.14-2.30 (3H), 3.77-3.86 (1H), 4.54-4.68 (2H), 7.06 (1H), 7.28-7.39 (2H), 7.64-7.69 (2H), 7.78 (1H), 7.96 (1H), 8.16-8.20 (2H).

LC-MS (Method 2): Rₜ=1.05 min; MS (ESIpos) m/z=362 [M+H]⁺.

### Example I-27

### 1-[2-({[3-(1-Benzofuran-2-yl)imidazo[1,2-b]pyridazin-6-yl]oxy}methyl)-morpholin-4-yl]ethanone

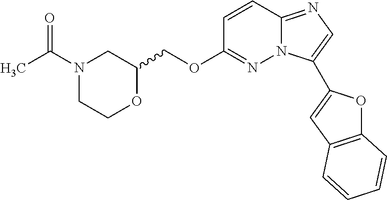 [SMILES: CC(=O)N1CCOC(COc2ccc3ncc(-c4cc5ccccc5o4)n3n2)C1]

To 80 mg (0.23 mmol) 3-(1-benzofuran-2-yl)-6-(morpholin-2-ylmethoxy)-imidazo[1,2-b]pyridazine in 2 mL dichloromethane were added 37 μL (0.46 mmol) pyridine and 43 μL (0.46 mmol) acetic anhydride. The mixture was stirred for 24 h at room temperature. 50 μL of water were added and the mixture was concentrated. The obtained crude product was purified by HPLC to give 47 mg of the title compound as solid material.

¹H-NMR (400 MHz, DMSO-d₆, 80° C.): δ [ppm]=2.03 (3H), 3.53 (1H), 3.94 (2H), 4.60 (2H), 7.04 (1H), 7.27-7.32 (1H), 7.32-7.37 (1H), 7.58-7.65 (2H), 7.75 (1H), 8.09-8.16 (2H).

LC-MS (Method 1): Rₜ=1.04 min; MS (ESIpos) m/z=392 [M+H]⁺.

### Example I-28

### N-(2-{[3-(1-Benzofuran-2-yl)imidazo[1,2-b]pyridazin-6-yl]oxy}ethyl)acetamide

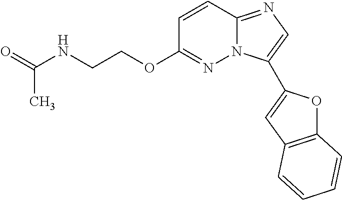 [SMILES: CC(=O)NCCOc1ccc2ncc(-c3cc4ccccc4o3)n2n1]

To 100 mg (0.34 mmol)) 2-{[3-(1-benzofuran-2-yl)imidazo[1,2-b]pyridazin-6-yl]oxy}-ethanamine in 4 mL dichloromethane were added 110 μL (1.36 mmol) pyridine and 64 μL (0.68 mmol) acetic anhydride. The mixture was stirred for 24 h at room temperature.

50 μL of water were added and the mixture was concentrated. The obtained crude product was purified by HPLC to give 44 mg of the title compound as solid material.

¹H-NMR (300 MHz, DMSO-d₆), δ [ppm]=1.82 (3H), 3.52-3.61 (2H), 4.52 (2H), 7.00 (1H), 7.30 (2H), 7.59-7.67 (2H), 7.71-7.77 (1H), 8.11-8.19 (2H).

LC-MS (Method 1): Rₜ=0.96 min; MS (ESIpos) m/z=337 [M+H]⁺.

### Example I-29

### (6S)-6-({[3-(1-Benzofuran-2-yl)imidazo[1,2-b]pyridazin-6-yl]oxy}methyl)-piperazin-2-one

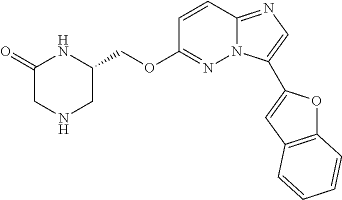 [SMILES: O=C1CNC[C@@H](COc2ccc3ncc(-c4cc5ccccc5o4)n3n2)N1]

In an ice bath, 220 mg (0.96 mmol) (6S)-4-(2,2-dimethylpropanoyl)-6-(hydroxymethyl)piperazin-2-one were added to 38 mg (0.96 mmol) sodium hydride (60% in mineral oil) in 4 mL anhydrous THF. After 15 min of stirring on the ice bath, 129 mg (0.48 mmol) of 6-chloro-3-(1-benzofuran-2-yl)imidazo[1,2-b]pyridazine were added. The ice bath was removed and the mixture was stirred for 16 h at 40° C.

The reaction mixture was poured into saturated aqueous ammonium chloride solution and extracted with ethyl acetate. The organic layer was dried over magnesium sulfate, and concentrated.

5 mL dichloromethane were added to the obtained crude product. The mixture was treated with 270 μL (2 mmol) trifluoro acetic acid and stirred for 24 h at room temperature.

Saturated aqueous sodium hydrogen carbonate solution was added. The mixture was extracted with dichloromethane. The organic layer was dried over sodium sulfate and evaporated. The crude product was purified by HPLC to yield 25 mg of the title compound as solid material.

¹H-NMR (400 MHz, DMSO-d₆): δ [ppm]=2.93 (1H), 3.07 (1H), 3.22 (2H), 3.79-3.86 (1H), 4.48-4.54 (1H), 4.55-4.60 (1H), 7.08 (1H), 7.29-7.33 (1H), 7.34-7.39 (1H), 7.66 (2H), 7.74-7.78 (1H), 7.93 (1H), 8.17-8.22 (2H).

LC-MS (Method 2): Rₜ=0.71 min; MS (ESIpos) m/z=364 [M+H]⁺.

### Example I-30

### N-[(2R)-2-{[3-(1-Benzofuran-2-yl)imidazo[1,2-b]pyridazin-6-yl]oxy}-2-(pyridin-3-yl)ethyl]-2-methoxyacetamide

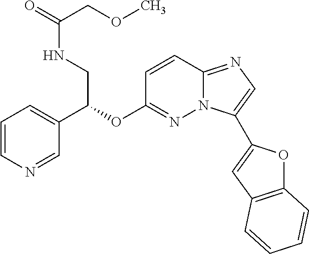 [SMILES: COCC(=O)NC[C@H](Oc1ccc2ncc(-c3cc4ccccc4o3)n2n1)c1cccnc1]

### Step 1:

At 0-5° C. 2.35 g (11.13 mmol) (1R)-2-amino-1-(pyridin-3-yl)ethanol dihydrochloride were added to 1.34 g (33.37 mmol) sodiumhydride (60% in mineral oil) in 75 mL anhydrous DMF. After 5 minutes of stirring on the ice bath, 1.50 g (5.56 mmol) 3-(1-benzofur-2-yl)-6-chloroimidazo[1,2-b]pyridazine were added. The ice bath was removed and it was stirred 2.5 h at room temperature. The reaction mixtures were poured into half saturated ammonium chloride solution. The layers were separated and the aqueous phase was extracted four times with ethyl acetate. The combined organic phases were washed with brine, dried over magnesium sulfate, and concentrated yielding 1.39 g (67%) (2R)-2-{[3-(1-benzofuran-2-yl)imidazo[1,2-b]pyridazin-6-yl]oxy}-2-(pyridin-3-yl)ethanamine.

¹H-NMR (300 MHz, CHLOROFORM-d), δ [ppm]=3.22-3.40 (2H), 6.02 (1H), 6.91 (1H), 7.07 (1H), 7.23-7.37 (3H, and chloroform signal), 7.51 (1H), 7.64-7.70 (1H), 7.81 (1H), 7.92 (1H), 8.11 (1H), 8.57 (1H), 8.83 (1H).

LC-MS (Method 2): Rₜ=0.75 min; MS (ESIpos) m/z=371 [M+H]⁺.

Step 2:

To 150 mg (0.40 mmol) (2R)-2-{[3-(1-benzofuran-2-yl)imidazo[1,2-b]pyridazin-6-yl]oxy}-2-(pyridin-3-yl)ethanamine in 1.5 mL anhydrous dichloromethane were added 141 μL (0.81 mmol) N-ethyl-N-isopropylpropan-2-amine and 9.9 mg (0.08 mmol) N,N-dimethylpyridin-4-amine. At 0° C. 74 μL (0.81 mmol) methoxyacetyl chloride were added. After 30 min at 0° C. the ice bath was removed and it was stirred 2 h at room temperature. 10 mL saturated ammonium chloride solution were added and it was extracted three times with dichloromethane. The combined organic phases were washed with saturated sodium hydrogencarbonate solution and brine, dried over magnesium sulfate and concentrated. The residue was purified by HPLC to give 77.7 mg (4%) of the title compound.

¹H-NMR (300 MHz, CHLOROFORM-d), δ [ppm]=3.36 (3H), 3.74-3.86 (1H), 3.90 (2H), 3-99-4.10 (1H), 6.19 (1H), 6.91 (1H), 6.99 (1H), 7.09 (1H), 7.27-7.37 (3H), 7.52 (1H), 7.69 (1H), 7.86 (1H), 7.95 (1H), 8.13 (1H), 8.59 (1H), 8.84 (1H).

LC-MS (Method 2): Rₜ=0.95 min; MS (ESIpos) m/z=443 [M+H]⁺.

### Example I-31

### 1-[(2S)-2-(2-{[3-(5-Chloro-1-benzofuran-2-yl)imidazo[1,2-b]pyridazin-6-yl]oxy}ethyl)pyrrolidin-1-yl]ethanone

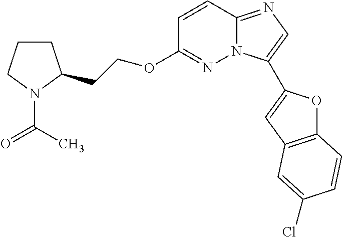 [SMILES: CC(=O)N1CCC[C@H]1CCOc1ccc2ncc(-c3cc4cc(Cl)ccc4o3)n2n1]

To 80 mg (0.21 mmol) 3-(5-chloro-1-benzofuran-2-yl)-6-{2-[(2S)-pyrrolidin-2-yl]ethoxy}imidazo[1,2-b]pyridazine in 3 mL dichloromethane were added 67 μL (0.84 mmol) pyridine and 79 μL (0.84 mmol) acetic anhydride. The mixture was stirred for 3 h at room temperature.

50 μL of water were added and the mixture was stirred for 5 min. Saturated aqueous sodium hydrogen carbonate solution was added and the mixture was extracted with ethyl acetate. The combined organic layers were dried over magnesium sulfate and evaporated. The obtained crude product was purified by flash chromatography and HPLC to yield 45 mg of the title compound as solid material.

¹H-NMR (300 MHz, DMSO-d₆), δ [ppm]=1.75-2.02 (3H), 2.21 (1H), 3.35-3.52 (2H), 4.19 (1H), 4.43-4.56 (2H), 6.97-7.05 (1H), 7.32 (1H), 7.60-7.72 (2H), 7.79 (1H), 8.07-8.18 (2H).

LC-MS (Method 3): Rₜ=1.31 min; MS (ESIpos) m/z=425 [M+H]⁺.

### Example I-32

### (5S)-5-({[3-(1-Benzofuran-2-yl)imidazo[1,2-b]pyridazin-6-yl]oxy}methyl)-1-methylpyrrolidin-2-one

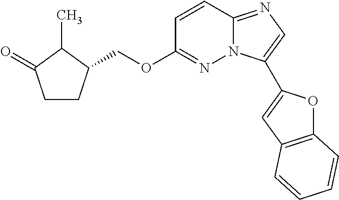 [SMILES: CC1C(=O)CC[C@H]1COc1ccc2ncc(-c3cc4ccccc4o3)n2n1]

In an ice bath, 572 mg (1.64 mmol) (5S)-5-({[3-(1-Benzofuran-2-yl)imidazo[1,2-b]pyridazin-6-yl]oxy}methyl)-pyrrolidin-2-one in 20 mL dichloromethane were treated with 197 mg (4.93 mmol) sodium hydride (60% dispersion in mineral oil). After 10 min, 400 μL (6.57 mmol) iodomethane were added, the ice bath removed and an stirring was continued for 16 h at room temperature. Again, 1 mL (16.4 mmol) iodomethane were added and stirring was continued for another 24 h.

The reaction mixture was poured into saturated aqueous ammonium chloride solution and extracted with ethyl acetate. The organic layer was washed with brine, dried over magnesium sulfate, and concentrated. The residue was purified by HPLC to give 75 mg of the title compound as solid material.

¹H-NMR (500 MHz, DMSO-d₆): _{d} [ppm]=1.97-2.06 (1H), 2.18-2.29 (1H), 2.40-2.49 (1H), 2.83 (2H), 4.05 (1H), 4.60 (1H), 4.74 (1H), 7.08 (1H), 7.28-7.40 (1H), 7.66 (1H), 7.69 (1H), 7.75 (1H), 8.16-8.22 (1H).

LCMS (Method 3): Rₜ=1.05 min; MS (ESIpos) m/z=363 [M+H]+.

### Example I-33

### 1-(2-{[3-(5-Methoxy-1-benzofuran-2-yl)imidazo[1,2-b]pyridazin-6-yl]oxy}ethyl)imidazolidin-2-one

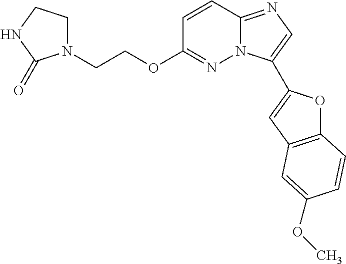 [SMILES: COc1ccc2oc(-c3cnc4ccc(OCCN5CCNC5=O)nn34)cc2c1]

In an ice bath, 62 mg (0.47 mmol) 1-(2-hydroxyethyl)imidazolidin-2-one were added to 16 mg (0.41 mmol) sodium hydride (60% in mineral oil) in 1.6 mL anhydrous THF. After 15 min of stirring on the ice bath, 70 mg (0.26 mmol) of 6-chloro-3-(5-methoxy-1-benzofuran-2-yl)imidazo[1,2-b]pyridazine were added. The ice bath was removed and the mixture was stirred for 17 h at room temperature.

The reaction mixture was poured into saturated aqueous ammonium chloride solution and extracted with ethyl acetate. The organic layer was dried over magnesium sulfate, and concentrated. The residue was purified by flash chromatography to yield 56 mg of the title compound as solid material.

¹H-NMR (300 MHz, DMSO-d₆), δ [ppm]=3.17-3.25 (2H), 3.44-3.52 (2H), 3.56 (2H), 3.78 (3H), 4.58 (2H), 6.38 (1H), 6.89 (1H), 7.01 (1H), 7.26 (1H), 7.51 (1H), 7.61 (1H), 8.10-8.18 (2H).

LC-MS (Method 3): Rₜ=1.01 min; MS (ESIpos) m/z=394 [M+H]⁺.

### Example I-34

### N-[2-{[3-(1-Benzofuran-2-yl)imidazo[1,2-b]pyridazin-6-yl]oxy}-1-(pyridin-3-yl)ethyl]acetamide

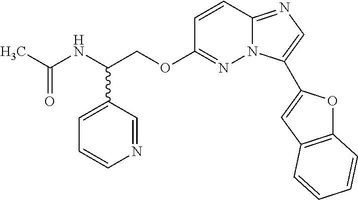 [SMILES: CC(=O)NC(COc1ccc2ncc(-c3cc4ccccc4o3)n2n1)c1cccnc1]

Step 1:

At 0-5° C. 204.9 mg (1.48 mmol) 2-amino-2-(pyridin-3-yl)ethanol were added to 59.3 mg (1.48 mmol) sodiumhydride (60% in mineral oil) in 7.5 mL anhydrous DMF. After 5 minutes of stirring on the ice bath, 200 mg (0.74 mmol) 3-(1-benzofur-2-yl)-6-chloroimidazo[1,2-b]pyridazine were added. The ice bath was removed and it was stirred over night at room temperature. The reaction mixtures were poured into half saturated ammonium chloride solution. Ethyl acetate was added, the layers were separated and the aqueous phase was extracted three times with ethyl acetate. The combined organic phases were washed with brine, dried over magnesium sulfate, and concentrated to yield 260 mg (94%) 2-{[3-(1-benzofuran-2-yl)imidazo[1,2-b]pyridazin-6-yl]oxy}-1-(pyridin-3-yl)ethanamine.

¹H-NMR (400 MHz, DMSO-d₆), δ [ppm]=4.39-4.45 (1H), 4.54-4.62 (2H), 6.95 (1H), 7.23-7.38 (3H), 7.56 (1H), 7.59-7.68 (2H), 7.90-7.94 (1H), 8.11-8.15 (2H), 8.46 (1H), 8.69 (1H).

LC-MS (Method 2): Rₜ=0.79 min; MS (ESIpos) m/z=371 [M+H]⁺.

Step 2:

To 260 mg (0.70 mmol) 2-{[3-(1-benzofuran-2-yl)imidazo[1,2-b]pyridazin-6-yl]oxy}-1-(pyridin-3-yl)ethanamine in 2.6 mL anhydrous dichloromethane and 62 μL (0.77 mmol) anhydrous pyridine were added 55 μL (0.77 mmol) acetanhydride at 0° C. It was stirred over night at room temperature. The reaction mixture was poured into icewater and the pH was adjusted to 3-4 with 2M sulfuric acid. After 1 h of stirring the insoluble material was filtered off, washed twice with water and twice with methanol. The solid was dried at 40° C. under vacuum to afford 9.4 mg (3%) product. The filtrate was concentrated and triturated with DMF. The insoluble material was filtered off, washed three time with methanol and dried at 40° C. under vacuum to yield 67 mg (23%) of the title compound.

¹H-NMR (400 MHz, DMSO-d₆), δ [ppm]=1.90 (3H), 4.69-4.79 (2H), 5.46-5.53 (1H), 6.98 (1H), 7.25-7.36 (2H), 7.37-7.41 (1H), 7.0-7.69 (3H), 7.84-7.88 (1H), 8.16 (2H), 8.48-8.51 (1H), 8.65-8.71 (2H).

LC-MS (Method 2): Rₜ=0.88 min; MS (ESIpos) m/z=413 [M+H]⁺.

### Example I-35

### N-[(2R)-2-{[3-(1-Benzofuran-2-yl)imidazo[1,2-b]pyridazin-6-yl]oxy}-2-(pyridin-3-yl)ethyl]acetamide

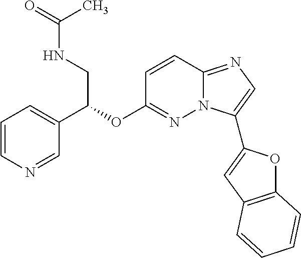 [SMILES: CC(=O)NC[C@H](Oc1ccc2ncc(-c3cc4ccccc4o3)n2n1)c1cccnc1]

Step 1:

At 0-5° C. 2.35 g (11.13 mmol) (1R)-2-amino-1-(pyridin-3-yl)ethanol dihydrochloride were added to 1.34 g (33.37 mmol) sodiumhydride (60% in mineral oil) in 75 mL anhydrous DMF. After 5 minutes of stirring on the ice bath, 1.50 g (5.56 mmol) 3-(1-benzofur-2-yl)-6-chloroimidazo[1,2-b]pyridazine were added. The ice bath was removed and it was stirred 2.5 h at room temperature. The reaction mixtures were poured into half saturated ammonium chloride solution. The layers were separated and the aqueous phase was extracted four times with ethyl acetate. The combined organic phases were washed with brine, dried over magnesium sulfate, and concentrated yielding 1.39 g (67%) (2R)-2-{[3-(1-benzofuran-2-yl)imidazo[1,2-b]pyridazin-6-yl]oxy}-2-(pyridin-3-yl)ethanamine.

¹H-NMR (300 MHz, CHLOROFORM-d), δ [ppm]=3.22-3.40 (2H), 6.02 (1H), 6.91 (1H), 7.07 (1H), 7.23-7.37 (3H, and chloroform signal), 7.51 (1H), 7.64-7.70 (1H), 7.81 (1H), 7.92 (1H), 8.11 (1H), 8.57 (1H), 8.83 (1H).

LC-MS (Method 2): Rₜ=0.75 min; MS (ESIpos) m/z=371 [M+H]⁺.

Step 2:

To 150 mg (0.40 mmol) (2R)-2-{[3-(1-benzofuran-2-yl)imidazo[1,2-b]pyridazin-6-yl]oxy}-2-(pyridin-3-yl)ethanamine in 1.5 mL anhydrous dichloromethane were added 141 μL (0.81 mmol) N-ethyl-N-isopropylpropan-2-amine and 9.9 mg (0.08 mmol) N,N-dimethylpyridin-4-amine. At 0° C. 57 μL (0.81 mmol) acetyl chloride were added. After 30 min at 0° C. the ice bath was removed and it was stirred 3 h at room temperature. 10 mL saturated ammonium chloride solution were added and it was extracted three times with dichloromethane. The combined organsich phases were washed with saturated sodium hydrogencarbonate solution and brine, dried over magnesium sulfate and concentrated. The residue was purified by HPLC to give 66.6 mg (37%) of the title compound.

¹H-NMR (300 MHz, CHLOROFORM-d), δ [ppm]=2.01 (3H), 3.73 (1H), 4.01 (1H), 5.95 (1H), 6.18 (1H), 6.89 (1H), 7.10 (1H), 7.27-7.38 (3H), 7.51 (1H), 7.69 (1H), 7.85 (1H), 7.95 (1H), 8.13 (1H), 8.58 (1H), 8.82 (1H).

LC-MS (Method 2): Rₜ=0.88 min; MS (ESIpos) m/z=413 [M+H]⁺.

### Example I-36

### 2-{[3-(1-Benzofuran-2-yl)imidazo[1,2-b]pyridazin-6-yl]oxy}-N,N-dimethyl-acetamide

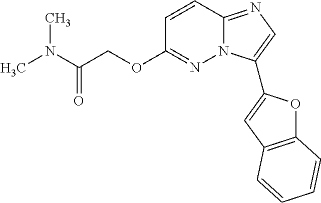 [SMILES: CN(C)C(=O)COc1ccc2ncc(-c3cc4ccccc4o3)n2n1]

35 mg (0.11 mmol) {[3-(1-benzofuran-2-yl)imidazo[1,2-b]pyridazin-6-yl]oxy}acetic acid in 3 mL DMF were treated with 24 μL (0.14 mmol) N-ethyl-N-isopropylpropan-2-amine, 52 mg (0.14 mmol) HATU and 62 μL (0.12 mmol) N,N-dimethylamine in THF (2 M solution). The mixture was stirred for 16 h at room temperature.

The solvent was evaporated and the residue was purified by HPLC to yield 3 mg of the title compound.

¹H-NMR (300 MHz, DMSO-d₆), δ [ppm]=2.86 (3H), 3.18 (3H), 5.26 (2H), 7.14 (1H), 7.31 (2H), 7.39 (1H), 7.60-7.72 (2H), 8.13-8.23 (2H).

LC-MS (Method 2): Rₜ=1.34 min; MS (ESIpos) m/z=337 [M+H]⁺.

### Example I-37

### 2-{[3-(1-Benzofuran-2-yl)imidazo[1,2-b]pyridazin-6-yl]oxy}-N-tert-butyl-acetamide

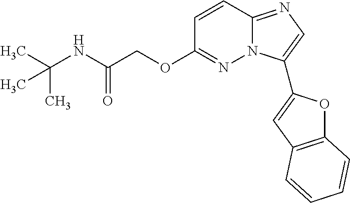 [SMILES: CC(C)(C)NC(=O)COc1ccc2ncc(-c3cc4ccccc4o3)n2n1]

35 mg (0.11 mmol) {[3-(1-benzofuran-2-yl)imidazo[1,2-b]pyridazin-6-yl]oxy}acetic acid in 3 mL DMF were treated with 24 μL (0.14 mmol) N-ethyl-N-isopropylpropan-2-amine, 52 mg (0.14 mmol) HATU and 14 μL (0.12 mmol) tert-butylamine. The mixture was stirred for 16 h at room temperature.

The solvent was evaporated and the residue was purified by HPLC to yield 14 mg of the title compound.

¹H-NMR (400 MHz, DMSO-d₆), δ [ppm]=1.23 (9H), 4.92 (2H), 7.13 (1H), 7.30-7.39 (2H), 7.63-7.68 (1H), 7.69-7.74 (2H), 8.06 (1H), 8.17 (1H), 8.20 (1H)

LC-MS (Method 2): Rₜ=1.16 min; MS (ESIpos) m/z=365 [M+H]⁺.

### Example I-38

### 3-{[3-(1-Benzofuran-2-yl)imidazo[1,2-b]pyridazin-6-yl]oxy}pyrrolidin-2-one

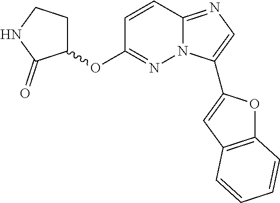 [SMILES: O=C1NCCC1Oc1ccc2ncc(-c3cc4ccccc4o3)n2n1]

At 0-5° C. 112.5 mg (1.11 mmol) 3-hydroxypyrrolidin-2-one were added to 44.5 mg (1.11 mmol) sodiumhydride (60% in mineral oil) in 7.5 mL anhydrous DMF. After 5 minutes of stirring on the ice bath, 150 mg (0.56 mmol) 3-(1-benzofur-2-yl)-6-chloroimidazo[1,2-b]pyridazine were added. The ice bath was removed and it was stirred 3 hours at room temperature. The reaction mixture was poured into half saturated ammonium chloride solution. The residue was filtered off and washed three times with water. The solid remainder was dissolved in methanol and concentrated under reduced pressure. This procedure was repeated. The residue was triturated in 4 mL methanol, filtered off and dried at 45° C. yielding 97.5 mg (52%) product.

¹H-NMR (300 MHz, DMSO-d₆), δ [ppm]=2.09-2.23 (1H), 2.72-2.84 (1H), 3.32-3.44 (2H), 5.68 (1H), 7.07 (1H), 7.30 (2H), 7.60-7.65 (1H), 7.68 (1H), 7.70-7.75 (1H), 8.15-8.22 (2H), 8.24-8.28 (1H).

LC-MS (Method 2): Rₜ=0.98 min; MS (ESIpos) m/z=334 [M+H]⁺.

### Example I-39

### 2-{[3-(1-Benzofuran-2-yl)imidazo[1,2-b]pyridazin-6-yl]oxy}-2-(pyridin-3-yl)acetamide

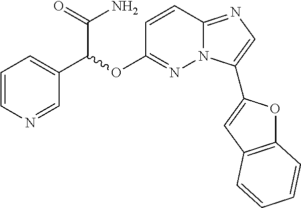 [SMILES: NC(=O)C(Oc1ccc2ncc(-c3cc4ccccc4o3)n2n1)c1cccnc1]

74 mg (1.85 mmol) sodiumhydride (60% in mineral oil) was washed with hexane and suspended in 12.5 mL of anhydrous DMF. At 0-5° C. 282 mg (1.85 mmol) 2-hydroxy-2-(pyridin-3-yl)acetamide were added. After 5 minutes of stirring on the ice bath, 250 mg (0.93 mmol) 3-(1-benzofur-2-yl)-6-chloroimidazo[1,2-b]pyridazine were added. The ice bath was removed and it was stirred over night at room temperature. The reaction mixture was poured into half saturated ammonium chloride solution, and extracted four times with ethyl acetate. The combined organic phases were washed with brine, dried over magnesium sulfate, and concentrated. The residue was purified by HPLC to yield 10 mg (3%) product.

¹H-NMR (600 MHz, DMSO-d₆), δ [ppm]=6.29 (1H), 7.21 (1H), 7.32-7.39 (2H), 7.50-7.53 (1H), 7.66-7.68 (1H), 7.73-7.76 (1H), 7.78 (1H), 8.08-8.10 (1H), 8.20 (1H), 8.22-8.24 (1H), 8.26 (1H), 8.31-8.34 (1H), 8.62-8.64 (1H), 8.92 (1H).

LC-MS (Method 2): Rₜ=0.90 min; MS (ESIpos) m/z=385 [M+H]⁺.

### Example I-40

### 1-[(2S)-2-(2-{[3-(5-Chloro-1-benzofuran-2-yl)imidazo[1,2-b]pyridazin-6-yl]-oxy}ethyl)pyrrolidin-1-yl]-2,2-dimethylpropan-1-one

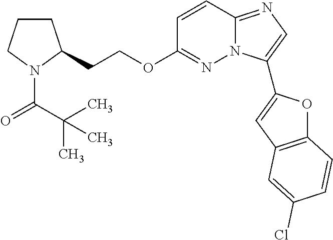 [SMILES: CC(C)(C)C(=O)N1CCC[C@H]1CCOc1ccc2ncc(-c3cc4cc(Cl)ccc4o3)n2n1]

To 80 mg (0.21 mmol) 3-(5-chloro-1-benzofuran-2-yl)-6-{2-[(2S)-pyrrolidin-2-yl]ethoxy}imidazo[1,2-b]pyridazine in 3 mL THF were added 67 μL (0.84 mmol) pyridine and 170 μL (0.84 mmol) 2,2-dimethylpropanoic anhydride. The mixture was stirred for 3 h at room temperature.

50 μL of water were added and the mixture was stirred for 5 min. Saturated aqueous sodium hydrogen carbonate solution was added and the mixture was extracted with ethyl acetate. The combined organic layers were dried over magnesium sulfate and evaporated. The obtained crude product was purified by flash chromatography and HPLC to yield 47 mg of the title compound as solid material.

¹H-NMR (300 MHz, DMSO-d₆), δ [ppm]=1.14 (9H), 1.66-2.00 (5H), 2.13-2.28 (1H), 3.44-3.57 (1H), 3.65 (1H), 4.21-4.32 (1H), 4.46 (2H), 6.99 (1H), 7.32 (1H), 7.60-7.69 (2H), 7.78 (1H), 8.14 (2H).

LC-MS (Method 3): Rₜ=1.58 min; MS (ESIpos) m/z=477 [M+H]⁺.

### Example I-41

### Cyclopropyl[(2R)-2-({[3-(4-methoxy-1-benzofuran-2-yl)imidazo[1,2-b]pyridazin-6-yl]oxy}methyl)morpholin-4-yl]methanone

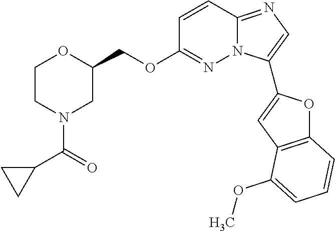 [SMILES: COc1cccc2oc(-c3cnc4ccc(OC[C@H]5CN(C(=O)C6CC6)CCO5)nn34)cc12]

To 200 mg (0.53 mmol) 3-(4-methoxy-1-benzofuran-2-yl)-6-[(2R)-morpholin-2-ylmethoxy]imidazo[1,2-b]pyridazine in 6.5 mL dichloromethane were added 85 μL (1.1 mmol) pyridine and 58 μL (0.63 mmol) cyclopropanecarbonyl chloride. The mixture was stirred for 16 h at 30° C.

The mixture was poured into brine and the mixture was extracted with dichloromethane. The combined organic layers were dried over sodium sulfate and evaporated. The obtained crude product was purified by HPLC to give 48 mg of the title compound as solid material.

¹H-NMR (400 MHz, DMSO-d₆), δ [ppm]=0.70 (4H), 1.96 (1H), 2.68-2.87 (1H), 3.37-3.61 (1H), 3.92 (5H), 4.14 (1H), 4.39 (1H), 4.55 (2H), 6.83 (1H), 7.06 (1H), 7.21-7.32 (2H), 7.54 (1H), 8.11-8.20 (2H).

LC-MS (Method 3): Rₜ=1.15 min; MS (ESIpos) m/z=449 [M+H]⁺.

### Example I-42

### (6R)-6-({[3-(1-Benzofuran-2-yl)imidazo[1,2-b]pyridazin-6yl]oxy}methyl)-piperazin-2-one

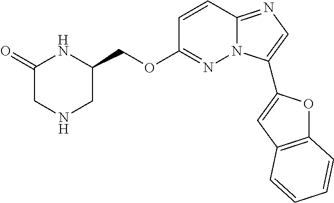 [SMILES: O=C1CNC[C@H](COc2ccc3ncc(-c4cc5ccccc5o4)n3n2)N1]

In an ice bath, 200 mg (0.87 mmol) (6R)-4-(2,2-dimethylpropanoyl)-6-(hydroxymethyl)piperazin-2-one were added to 35 mg (0.87 mmol) sodium hydride (60% in mineral oil) in 4 mL anhydrous THF. After 15 min of stirring on the ice bath, 117 mg (0.43 mmol) of 6-chloro-3-(1-benzofuran-2-yl)imidazo[1,2-b]pyridazine were added. The ice bath was removed and the mixture was stirred for 16 h at 40° C.

The reaction mixture was poured into brine and extracted with ethyl acetate. The organic layer was dried over magnesium sulfate, and concentrated.

5 mL dichloromethane were added to the obtained crude product. The mixture was treated with 270 μL (2 mmol) trifluoroacetic acid and stirred for 24 h at room temperature. Again, 4 mL of dichloromethane and 1 mL of methanol were added and stirring at room temperature was continued for 6 h. Another 100 μL trifluoroacetic acid were added and the mixture was stirred for 48 h at room temperature. Once more, 2 mL trifluoroacetic acid were added and stirring at room temperature was continued for 4 h.

15 mL of a 1 M ammonia solution in water was added. The mixture was extracted with ethyl acetate. The organic layer was washed with brine, dried over sodium sulfate and evaporated. The crude product was purified by HPLC to yield 27 mg of the title compound as solid material.

¹H-NMR (400 MHz, DMSO-d₆): δ [ppm]=3.04 (1H), 3.24 (2H), 3.93 (1H), 4.49-4.65 (2H), 7.09 (1H), 7.29-7.34 (1H), 7.34-7.40 (1H), 7.64-7.69 (2H), 7.73-7.78 (1H), 8.13 (1H), 8.19 (1H), 8.21 (1H).

LC-MS (Method 3): Rₜ=0.72 min; MS (ESIpos) m/z=364 [M+H]⁺.

### Example I-43

### [(2S)-2-(2-{[3-(1-Benzofuran-2-yl)imidazo[1,2-b]pyridazin-6-yl]oxy}ethyl)pyrrolidin-1-yl](thiophen-2-yl)methanone

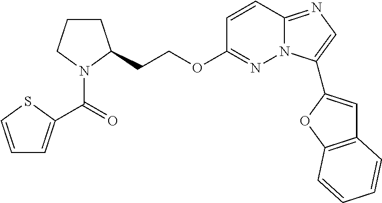 [SMILES: O=C(c1cccs1)N1CCC[C@H]1CCOc1ccc2ncc(-c3cc4ccccc4o3)n2n1]

To 25 mg (0.195 mmol) thiophene-2-carboxylic acid in 350 μL DMF were added 52 mg (0.15 mmol) 3-(1-benzofuran-2-yl)-6-{2-[pyrrolidin-2-yl]ethoxy}-imidazo[1,2-b]-pyridazine in 1 mL DMF, 70 mg (0.54 mmol) N-ethyl-N-isopropylpropan-2-amine in 1 mL DMF and 84 mg (0.195 mmol) COMU (0.4 M solution in DMF). The mixture was shaken at room temperature over night.

The solvent was evaporated and the obtained crude product was purified by HPLC to give 19 mg of the title compound as solid material.

LC-MS (Method 4): Rₜ=1.29 min; MS (ESIpos) m/z=405 [M+H]+.

The examples in the following table were prepared in analogy to example I-43.

### (LCMS Data Obtained Using Method 4):

| | | | MS | |
|---|---|---|---|---|
| | | | (ESIpos) | Retention |
| Exam- | | | m/z | time |
| ple No | Structure | Name | [M + H]+ | [min] |
| I-44 | 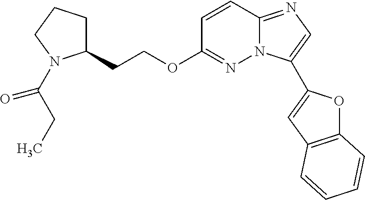 [SMILES: CCC(=O)N1CCC[C@H]1CCOc1ccc2ncc(-c3cc4ccccc4o3)n2n1] | 1-[(2S)-2-(2-{[3-(1- Benzofuran-2- yl)imidazo[1,2-b]- pyridazin-6- yl]oxy}ethyl)pyrrolidin- 1-yl]propan-1-one | 405 | 1.29 |
| I-45 | 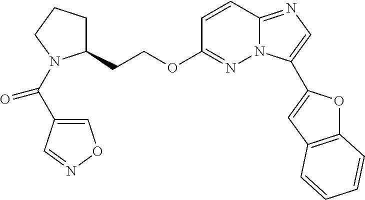 [SMILES: O=C(c1cnoc1)N1CCC[C@H]1CCOc1ccc2ncc(-c3cc4ccccc4o3)n2n1] | [(2S)-2-(2-{[3-(1- Benzofuran-2- yl)imidazo[1,2-b]- pyridazin-6- yl]oxy}ethyl)pyrrolidin- 1-yl](1,2-oxazol-4- yl)methanone | 444 | 1.34 |
| I-46 | 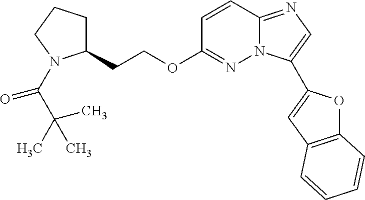 [SMILES: CC(C)(C)C(=O)N1CCC[C@H]1CCOc1ccc2ncc(-c3cc4ccccc4o3)n2n1] | 1-[(2S)-2-(2-{[3-(1- Benzofuran-2- yl)imidazo[1,2-b]-pyridazin- 6-yl]oxy}ethyl)pyrrolidin- 1-yl]-2,2-dimethylpropan- 1-one | 433 | 1.47 |
| I-47 | 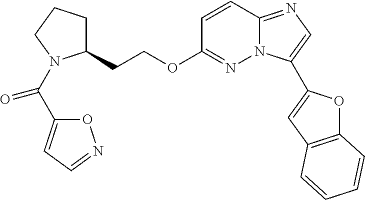 [SMILES: O=C(c1ccno1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](1,2-oxazol-5- yl)methanone | 444 | 1.27 |
| I-48 | 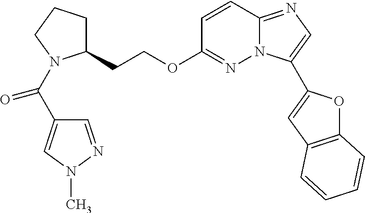 [SMILES: Cn1cc(C(=O)N2CCC[C@H]2CCOc2ccc3ncc(-c4cc5ccccc5o4)n3n2)cn1] | [(2S)-2-(2-{[3-(1- Benzofuran-2- yl)imidazo[1,2-b]-pyridazin- 6-yl]oxy}ethyl)pyrrolidin-1- yl](1-methyl-1H-pyrazol-4- yl)methanone | 457 | 1.18 |
| I-49 | 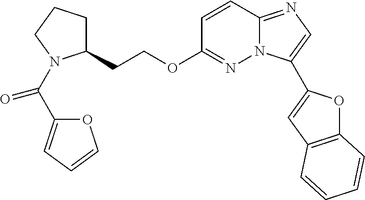 [SMILES: O=C(c1ccco1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](furan-2-yl)methanone | 443 | 1.33 |
| I-50 | 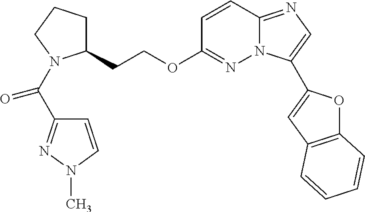 [SMILES: Cn1ccc(C(=O)N2CCC[C@H]2CCOc2ccc3ncc(-c4cc5ccccc5o4)n3n2)n1] | [(2S)-2-(2-{[3-(1- Benzofuran-2- yl)imidazo[1,2-b]-pyridazin- 6-yl]oxy}ethyl)pyrrolidin-1- yl](1-methyl-1H-pyrazol-3- yl)methanone | 457 | 1.26 |
| I-51 | 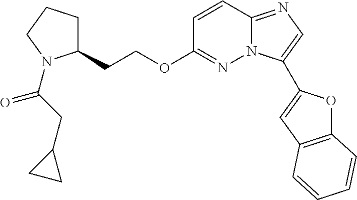 [SMILES: O=C(CC1CC1)N1CCC[C@H]1CCOc1ccc2ncc(-c3cc4ccccc4o3)n2n1] | 1-[(2S)-2-(2-{[3-(1- Benzofuran-2- yl)imidazo[1,2-b]-pyridazin- 6-yl]oxy}ethyl)pyrrolidin-1- yl]-2-cyclopropylethanone | 431 | 1.36 |
| I-52 | 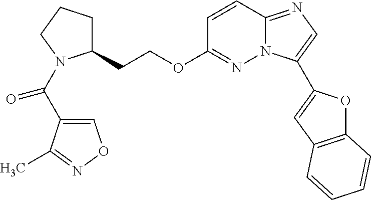 [SMILES: Cc1nocc1C(=O)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](3-methyl-1,2-oxazol-4- yl)methanone | 458 | 1.27 |
| I-53 | 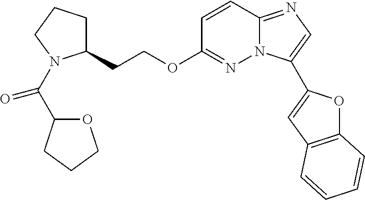 [SMILES: O=C(C1CCCO1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](tetrahydrofuran-2- yl)methanone | 447 | 1.25 |
| I-54 | 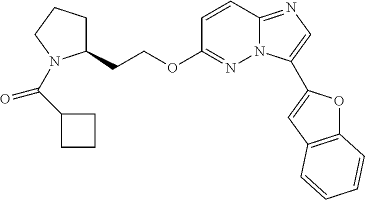 [SMILES: O=C(C1CCC1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](cyclobutyl)methanone | 431 | 1.4 |
| I-55 | 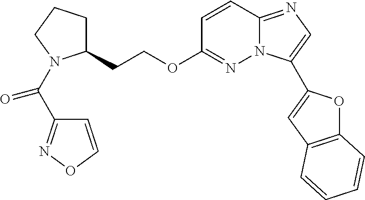 [SMILES: O=C(c1ccon1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](1,2-oxazol-3- yl)methanone | 444 | 1.3 |
| I-56 | 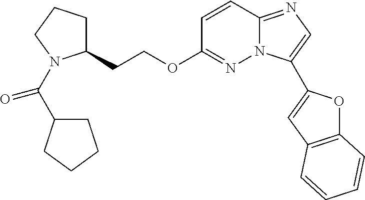 [SMILES: O=C(C1CCCC1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](cyclopentyl)-methanone | 445 | 1.45 |
| I-57 | 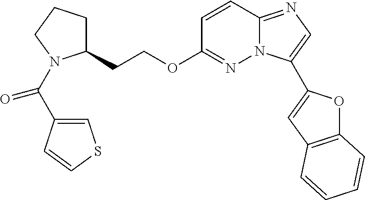 [SMILES: O=C(c1ccsc1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](thiophen-3- yl)methanone | 459 | 1.36 |
| I-58 | 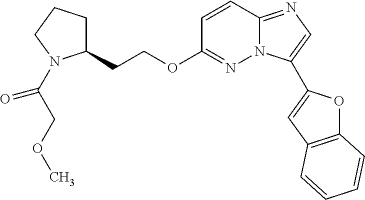 [SMILES: COCC(=O)N1CCC[C@H]1CCOc1ccc2ncc(-c3cc4ccccc4o3)n2n1] | 1-[(2S)-2-(2-{[3-(1- Benzofuran-2- yl)imidazo[1,2-b]-pyridazin- 6-yl]oxy}ethyl)pyrrolidin-1- yl]-2-methoxyethanone | 421 | 1.2 |
| I-59 | 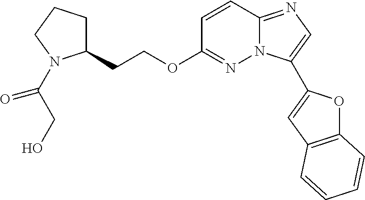 [SMILES: O=C(CO)N1CCC[C@H]1CCOc1ccc2ncc(-c3cc4ccccc4o3)n2n1] | 1-[(2S)-2-(2-{[3-(1- Benzofuran-2- yl)imidazo[1,2-b]-pyridazin- 6-yl]oxy}ethyl)pyrrolidin-1- yl]-2-hydroxyethanone | 407 | 1.15 |
| I-60 | 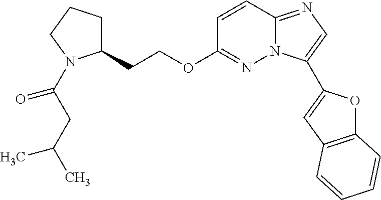 [SMILES: CC(C)CC(=O)N1CCC[C@H]1CCOc1ccc2ncc(-c3cc4ccccc4o3)n2n1] | 1-[(2S)-2-(2-{[3-(1- Benzofuran-2- yl)imidazo[1,2-b]-pyridazin- 6-yl]oxy}ethyl)pyrrolidin-1- yl]-3-methylbutan-1-one | 433 | 1.42 |
| I-61 | 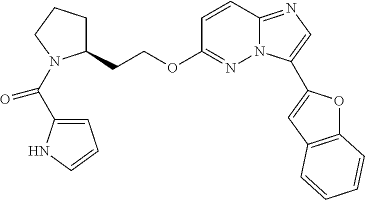 [SMILES: O=C(c1ccc[nH]1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](1H-pyrrol-2- yl)methanone | 442 | 1.34 |
| I-62 | 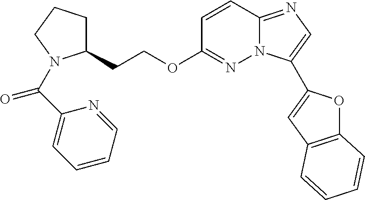 [SMILES: O=C(c1ccccn1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](pyridin-2-yl)methanone | 454 | 1.26 |
| I-63 | 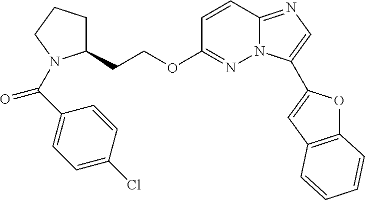 [SMILES: O=C(c1ccc(Cl)cc1)N1CCC[C@H]1CCOc1ccc2ncc(-c3cc4ccccc4o3)n2n1] | [(2S)-2-(2-{[3-(1- Benzofuran-2- yl)imidazo[1,2-b]-pyridazin- 6-yl]oxy}ethyl)pyrrolidin-1- yl](4- chlorophenyl)methanone | 488 | 1.45 |

### Example I-64

### N-(2-{[3-(1-Benzofuran-2-yl)imidazo[1,2-b]-pyridazin-6-yl]oxy}ethyl)thiophene-2-carboxamide

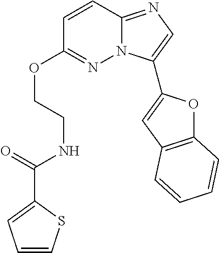 [SMILES: O=C(NCCOc1ccc2ncc(-c3cc4ccccc4o3)n2n1)c1cccs1]

To 25 mg (0.195 mmol) thiophene-2-carboxylic acid in 350 μL DMF were added 52 mg (0.15 mmol) 3-(1-benzofuran-2-yl)-6-{2-[pyrrolidin-2-yl]ethoxy}-imidazo[1,2-b]-pyridazine in 1 mL DMF, 70 mg (0.54 mmol) N-ethyl-N-isopropylpropan-2-amine in 1 mL DMF and 84 mg (0.195 mmol) COMU (0.4 M solution in DMF). The mixture was shaken at room temperature over night.

The solvent was evaporated and the obtained crude product was purified by HPLC to give 19 mg of the title compound as solid material.

LC-MS (Method 4): Rₜ=1.29 min; MS (ESIpos) m/z=405 [M+H]+.

The examples in the following table were prepared in analogy to example I-64.

### (LCMS Data Obtained Using Method 4):

| | | | MS | |
|---|---|---|---|---|
| | | | (ESIpos) | Retention |
| Exam- | | | m/z | time |
| ple No | Structure | Name | [M + H]+ | [min] |
| I-65 | 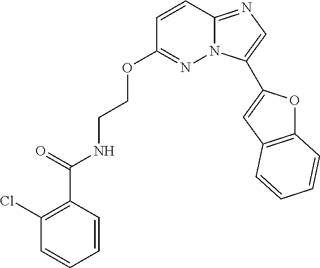 [SMILES: O=C(NCCOc1ccc2ncc(-c3cc4ccccc4o3)n2n1)c1ccccc1Cl] | N-(2-{[3-(1-Benzofuran-2- yl)imidazo[1,2-b]-pyridazin- 6-yl]oxy}ethyl)-2- chlorobenzamide | 433 | 1.25 |
| I-66 | 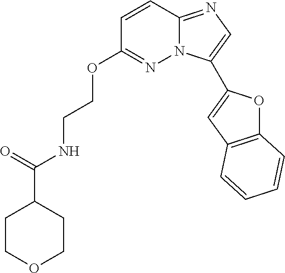 [SMILES: O=C(NCCOc1ccc2ncc(-c3cc4ccccc4o3)n2n1)C1CCOCC1] | N-(2-{[3-(1-Benzofuran-2- yl)imidazo[1,2-b]-pyridazin- 6-yl]oxy}ethyl)tetrahydro- 2H-pyran-4-carboxamide | 407 | 1.07 |
| I-67 | 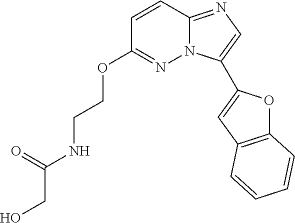 [SMILES: O=C(CO)NCCOc1ccc2ncc(-c3cc4ccccc4o3)n2n1] | N-(2-{[3-(1-Benzofuran-2- yl)imidazo[1,2-b]-pyridazin- 6-yl]oxy}ethyl)-2- hydroxyacetamide | 353 | 0.96 |
| I-68 | 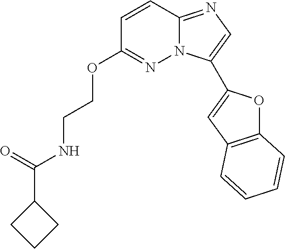 [SMILES: O=C(NCCOc1ccc2ncc(-c3cc4ccccc4o3)n2n1)C1CCC1] | N-(2-{[3-(1-Benzofuran-2- yl)imidazo[1,2-b]-pyridazin- 6- yl]oxy}ethyl)cyclobutane- carboxamide | 377 | 1.18 |
| I-69 | 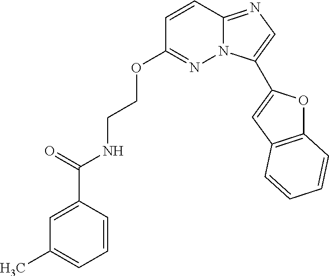 [SMILES: Cc1cccc(C(=O)NCCOc2ccc3ncc(-c4cc5ccccc5o4)n3n2)c1] | N-(2-{[3-(1-Benzofuran-2- yl)imidazo[1,2-b]-pyridazin- 6-yl]oxy}ethyl)-3- methylbenzamide | 413 | 1.28 |
| I-70 | 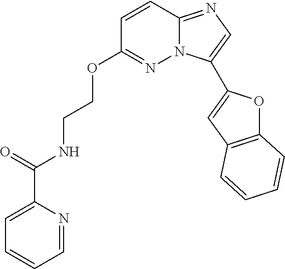 [SMILES: O=C(NCCOc1ccc2ncc(-c3cc4ccccc4o3)n2n1)c1ccccn1] | N-(2-{[3-(1-Benzofuran-2- yl)imidazo[1,2-b]-pyridazin- 6-yl]oxy}ethyl)pyridine-2- carboxamide | 400 | 1.21 |
| I-71 | 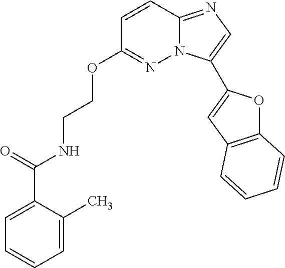 [SMILES: Cc1ccccc1C(=O)NCCOc1ccc2ncc(-c3cc4ccccc4o3)n2n1] | N-(2-{[3-(1-Benzofuran-2- yl)imidazo[1,2-b]-pyridazin- 6-yl]oxy}ethyl)-2- methylbenzamide | 413 | 1.26 |
| I-72 | 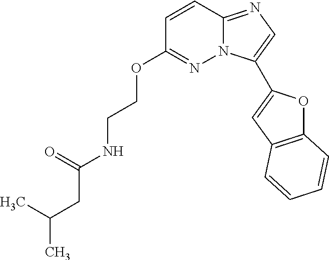 [SMILES: CC(C)CC(=O)NCCOc1ccc2ncc(-c3cc4ccccc4o3)n2n1] | N-(2-{[3-(1-Benzofuran-2- yl)imidazo[1,2-b]-pyridazin- 6-yl]oxy}ethyl)-3- methylbutanamide | 379 | 1.21 |
| I-73 | 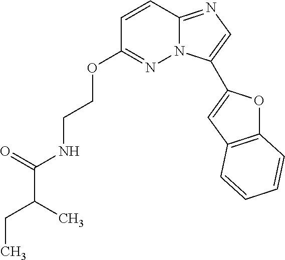 [SMILES: CCC(C)C(=O)NCCOc1ccc2ncc(-c3cc4ccccc4o3)n2n1] | N-(2-{[3-(1-Benzofuran-2- yl)imidazo[1,2-b]-pyridazin- 6-yl]oxy}ethyl)-2- methylbutanamide | 379 | 1.21 |
| I-74 | 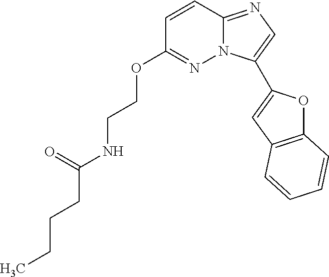 [SMILES: CCCCC(=O)NCCOc1ccc2ncc(-c3cc4ccccc4o3)n2n1] | N-(2-{[3-(1-Benzofuran-2- yl)imidazo[1,2-b]-pyridazin- 6-yl]oxy}ethyl)pentanamide | 379 | 1.21 |
| I-75 | 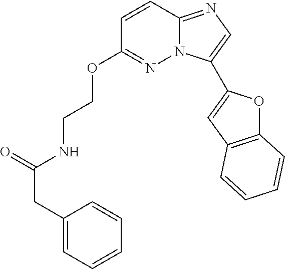 [SMILES: O=C(Cc1ccccc1)NCCOc1ccc2ncc(-c3cc4ccccc4o3)n2n1] | N-(2-{[3-(1-Benzofuran-2- yl)imidazo[1,2-b]-pyridazin- 6-yl]oxy}ethyl)-2- phenylacetamide | 413 | 1.22 |
| I-76 | 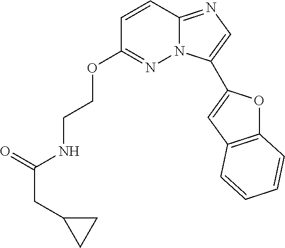 [SMILES: O=C(CC1CC1)NCCOc1ccc2ncc(-c3cc4ccccc4o3)n2n1] | N-(2-{[3-(1-Benzofuran-2- yl)imidazo[1,2-b]-pyridazin- 6-yl]oxy}ethyl)-2- cyclopropylacetamide | 377 | 1.15 |
| I-77 | 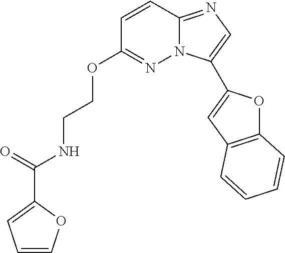 [SMILES: O=C(NCCOc1ccc2ncc(-c3cc4ccccc4o3)n2n1)c1ccco1] | N-(2-{[3-(1-Benzofuran-2- yl)imidazo[1,2-b]-pyridazin- 6-yl]oxy}ethyl)furan-2- carboxamide | 389 | 1.14 |
| I-78 | 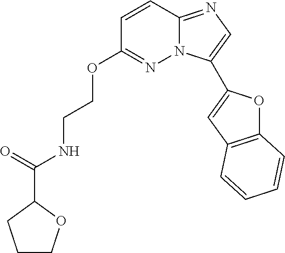 [SMILES: O=C(NCCOc1ccc2ncc(-c3cc4ccccc4o3)n2n1)C1CCCO1] | N-(2-{[3-(1-Benzofuran-2- yl)imidazo[1,2-b]-pyridazin- 6- yl]oxy}ethyl)tetrahydrofuran- 2-carboxamide | 393 | 1.11 |
| I-79 | 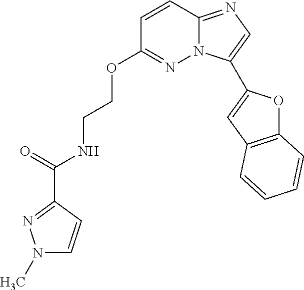 [SMILES: Cn1ccc(C(=O)NCCOc2ccc3ncc(-c4cc5ccccc5o4)n3n2)n1] | N-(2-{[3-(1-Benzofuran-2- yl)imidazo[1,2-b]-pyridazin- 6-yl]oxy}ethyl)-1-methyl- 1H-pyrazole-3-carboxamide | 403 | 1.1 |

### Example I-80

### N-[(2R)-2-{[3-(1-Benzofuran-2-yl)imidazo[1,2-b]pyridazin-6-yl]oxy}-propyl]acetamide

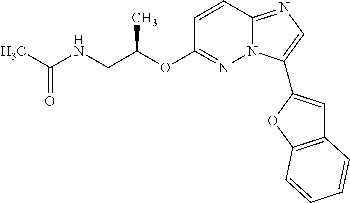 [SMILES: CC(=O)NC[C@@H](C)Oc1ccc2ncc(-c3cc4ccccc4o3)n2n1]

To 1.5 g (4.87 mmol) (2R)-2-{[3-(1-benzofuran-2-yl)imidazo[1,2-b]pyridazin-6-yl]oxy}propan-1-amine in 20 mL dichloromethane were added 1.58 mL (19.5 mmol) pyridine and 0.92 mL (9.73 mmol) acetic anhydride. The mixture was stirred for 3.5 h at room temperature.

1 mL of water was added and the mixture was concentrated under reduced pressure. The obtained crude product was digested in methanol at 60° C. The precipitate was filtered off and washed with methanol and hexane to give 1.34 g of the title compound as solid material.

¹H-NMR (300 MHz, DMSO-d₆), δ [ppm]=1.44 (3H), 1.82 (3H), 3.43-3.55 (2H), 5.32-5.44 (1H), 6.98 (1H), 7.26-7.40 (2H), 7.62-7.68 (2H), 7.73-7.79 (1H), 8.10-8.21 (3H).

LC-MS (Method 3): Rₜ=1.06 min; MS (ESIpos) m/z=351 [M+H]⁺.

### Example II-1

### 3-[4-(Morpholin-4-yl)-1-benzofuran-2-yl]-6-[(2R)-morpholin-2-ylmethoxy]-imidazo[1,2-b]pyridazine

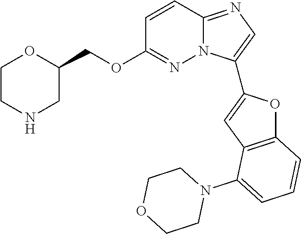 [SMILES: c1cc(N2CCOCC2)c2cc(-c3cnc4ccc(OC[C@H]5CNCCO5)nn34)oc2c1]

To 150 mg (0.48 mmol) 3-bromo-6-[(2R)-morpholin-2-ylmethoxy]imidazo[1,2-b]-pyridazine were added 375 mg (calculated as 236.7 mg pure material) (0.96 mmol) [4-(morpholin-4-yl)-1-benzofuran-2-yl]boronic acid dissolved in 7 mL 1-propanol, 0.72 mL (1.44 mmol) 2M aqueous potassium carbonate solution, 12.6 mg (0.05 mmol) triphenylphosphine and 34 mg (0.05 mmol) PdCl₂(PPh₃)₂. It was stirred 2 h at 130° C. bath temperature. The reaction mixture was cooled to room temperature and diluted with dichloromethane. The organic phase was washed with water, dried over magnesium sulfate and concentrated. The residue was purified by HPLC to give 35 mg (17%) of the title compound as solid material.

¹H-NMR (300 MHz, DMSO-d₆), δ [ppm]=2.50-2.60 (1H), 2.60-2.75 (2H), 2.90-2.98 (1H), 3.11-3.21 (4H), 3.44-3.54 (1H), 3.71-3.88 (6H), 4.33-4.43 (2H), 6.72 (1H), 7.04 (1H), 7.22 (2H), 7.55 (1H), 8.09-8.18 (2H).

LC-MS (Method 6): Rₜ=0.84 min; MS (ESIpos) m/z=436 [M+H]⁺.

### Example II-2

### (2S)-1-({3-[4-(Morpholin-4-yl)-1-benzofuran-2-yl]imidazo[1,2-b]pyridazin-6-yl}oxy)propan-2-amine

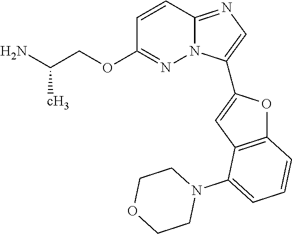

To 150 mg (0.55 mmol) (2S)-1-[(3-bromoimidazo[1,2-b]pyridazin-6-yl)oxy]propan-2-amine were added 430 mg (calculated as 273.4 mg pure material) (1.11 mmol) [4-(morpholin-4-yl)-1-benzofuran-2-yl]boronic acid dissolved in 8 mL 1-propanol, 0.83 mL (1.66 mmol) 2M aqueous potassiumcarbonate solution, 14.5 mg (0.06 mmol) triphenylphosphine and 39 mg (0.06 mmol) PdCl₂(PPh₃)₂. It was stirred 2 h at 130° C. bath temperature. The reaction mixture was cooled to room temperature and diluted with dichloromethane. The organic phase was washed with water, dried over magnesium sulfate and concentrated. The residue was purified by HPLC to give 26 mg (12%) of the title compound as solid material.

¹H-NMR (300 MHz, DMSO-d₆), δ [ppm]=1.14 (3H), 3.13-3.20 (4H), 3.76-3.88 (4H), 4.17-4.30 (2H), 6.74 (1H), 7.02 (1H), 7.20-7.26 (2H), 7.58 (1H), 8.12 (1H), 8.15 (1H).

LC-MS (Method 6): Rₜ=0.93 min; MS (ESIpos) m/z=394 [M+H]⁺.

### Example III-3

### tert-Butyl [trans-3-({3-[4-(morpholin-4-yl)-1-benzofuran-2-yl]imidazo[1,2-b]-pyridazin-6-yl}oxy)cyclobutyl]carbamate

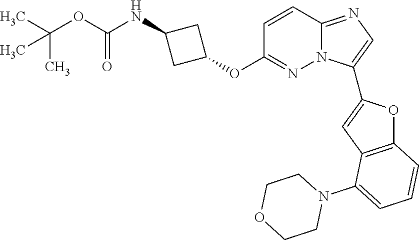 [SMILES: CC(C)(C)OC(=O)N[C@H]1C[C@H](Oc2ccc3ncc(-c4cc5c(N6CCOCC6)cccc5o4)n3n2)C1]

To 150 mg (0.39 mmol) tert-butyl {trans-3-[(3-bromoimidazo[1,2-b]pyridazin-6-yl)oxy]cyclobutyl}carbamate were added 306 mg (calculated as 193.4 mg pure material) (0.78 mmol) [4-(morpholin-4-yl)-1-benzofuran-2-yl]boronic acid dissolved in 5.7 mL 1-propanol, 0.59 mL (1.18 mmol) 2M aqueous potassiumcarbonate solution, 10.2 mg (0.04 mmol) triphenylphosphine and 27.6 mg (0.04 mmol) PdCl₂(PPh₃)₂. It was stirred 2 h at 130° C. bath temperature. The reaction mixture was cooled to room temperature and diluted with dichloromethane. The organic phase was washed with water, dried over magnesium sulfate and concentrated. The residue was purified by HPLC to give 18.3 mg (9%) of the title compound as solid material.

¹H-NMR (400 MHz, DMSO-d₆), δ [ppm]=2.49-2.65 (3H), 3.14-3.21 (4H), 3.83-3.91 (4H), 4.19-4.31 (1H), 5.27-5.33 (1H), 6.71-6.77 (1H), 7.00-7.05 (1H), 7.23 (2H), 7.40-7.49 (2H), 8.11 (1H), 8.15 (1H).

LC-MS (Method 6): Rₜ=1.40 min; MS (ESIpos) m/z=506 [M+H]⁺.

### Example II-4

### trans-3-({3-[4-(Morpholin-4-yl)-1-benzofuran-2-yl]imidazo[1,2-b]pyridazin-6-yl}oxy)cyclobutanamine

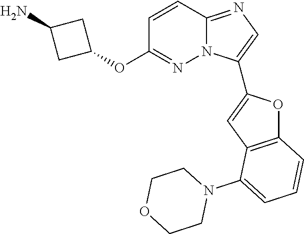 [SMILES: N[C@H]1C[C@H](Oc2ccc3ncc(-c4cc5c(N6CCOCC6)cccc5o4)n3n2)C1]

To 538 mg (0.69 mmol) of crude tert-butyl [trans-3-({3-[4-(morpholin-4-yl)-1-benzofuran-2-yl]imidazo[1,2-b]-pyridazin-6-yl}oxy)cyclobutyl]carbamate in 7 mL dichloromethane were added 7 mL (91 mmol) TFA. The mixture was stirred for 15 min at room temperature. 7 mL (91 mmol) ammonia (25% in water) were added. The mixture was extracted with dichloromethane. The organic layer was dried over sodium sulphate and evaporated. The residue was purified by HPLC to give 66 mg of the title compound as solid material.

¹H-NMR (300 MHz, DMSO-d₆), δ [ppm]=2.25 (2H), 3.16 (4H), 3.70 (1H), 3.85-3.94 (4H), 5.28-5.39 (1H), 6.74 (1H), 7.00 (1H), 7.23 (2H), 7.48 (1H), 8.09-8.17 (2H).

LC-MS (Method 6): Rₜ=0.81 min; MS (ESIpos) m/z=406 [M+H]⁺.

### Example II-5

### (5R)-5-[({3-[4-(Morpholin-4-yl)-1-benzofuran-2-yl]imidazo[1,2-b]pyridazin-6-yl}oxy)methyl]pyrrolidin-2-one

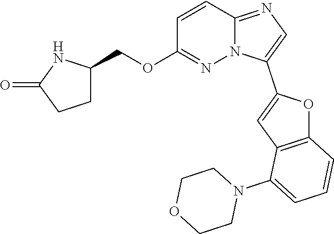 [SMILES: O=C1CC[C@H](COc2ccc3ncc(-c4cc5c(N6CCOCC6)cccc5o4)n3n2)N1]

To 150 mg (0.48 mmol) (5R)-5-{[(3-bromoimidazo[1,2-b]pyridazin-6-yl)oxy]-methyl}pyrrolidin-2-one were added 377 mg (calculated as 238.2 mg pure material) (0.96 mmol) [4-(morpholin-4-yl)-1-benzofuran-2-yl]boronic acid dissolved in 7 mL 1-propanol, 0.72 mL (1.44 mmol) 2M aqueous potassiumcarbonate solution, 12.6 mg (0.05 mmol) triphenylphosphine and 34 mg (0.05 mmol) PdCl₂(PPh₃)₂. It was stirred 2 h at 100° C. bath temperature. The reaction mixture was cooled to room temperature and diluted with dichloromethane. The organic phase was washed with water, dried over magnesium sulfate and concentrated. The residue was dissolved in warm DMF/DMSO. The solution was cooled to room temperature and filtered. After a week at room temperature the solid material was decanted and stirred in methanol. The product was filtered off and washed twice with methanol to yield 33 mg (16%) of the compound.

¹H-NMR (300 MHz, DMSO-d₆), δ [ppm]=1.83-1.97 (1H), 2.10-2.37 (3H), 3.13-3.21 (4H), 3.76-3.86 (4H), 3.96-4.06 (1H), 4.30-4.38 (1H), 4.44-4.52 (1H), 6.73 (1H), 7.02 (1H), 7.18-7.27 (2H), 7.59 (1H), 7.87-7.92 (1H), 8.13 (1H), 8.17 (1H).

LC-MS (Method 6): Rₜ=1.06 min; MS (ESIpos) m/z=434 [M+H]⁺.

### Example II-6

### 3-{4-[(2R)-2-(Methoxymethyl)pyrrolidin-1-yl]-1-benzofuran-2-yl}-6-[3-(methyl-sulfonyl)propoxy]imidazo[1,2-b]pyridazine

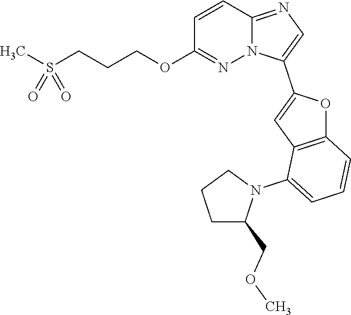 [SMILES: COC[C@H]1CCCN1c1cccc2oc(-c3cnc4ccc(OCCCS(C)(=O)=O)nn34)cc12]

158 mg (0.47 mmol) 3-bromo-6-[3-(methylsulfonyl)propoxy]imidazo[1,2-b]-pyridazine, 340 mg (0.8 mmol) {4-[(2R)-2-(methoxymethyl)pyrrolidin-1-yl]-1-benzofuran-2-yl}boronic acid (approximately 65% pure), 12 mg (47 μmol) triphenylphosphine, 27 mg (47 μmol) Pddba₂ and 0.71 mL (1.4 mmol) potassium carbonate (c=2 mol/L in water) in 5.5 mL n-propanol were heated to reflux for 2 h.

The mixture was poured into water and extracted with dichloromethane. The organic layer was dried over sodium sulfate and evaporated. The residue was digested in a 1:1 mixture of dichloromethane and methanol to give 78 mg of the title compound as solid material.

¹H-NMR (300 MHz, DMSO-d₆), δ [ppm]=1.91-2.06 (2H), 2.19-2.32 (2H), 3.01 (3H), 3.18-3.26 (4H), 3.38-3.53 (2H), 3.70 (1H), 4.24 (1H), 4.48-4.65 (2H), 6.35 (1H), 6.86 (1H), 6.98 (1H), 7.07-7.16 (1H), 7.82 (1H), 8.07 (1H), 8.14 (1H).

LC-MS (Method 7): Rₜ=1.26 min; MS (ESIpos) m/z=485 [M+H]⁺.

### Example II-7

### 3-{4-[(2R)-2-(Methoxymethyl)pyrrolidin-1-yl]-1-benzofuran-2-yl}-6-[(2R)-morpholin-2-ylmethoxy]imidazo[1,2-b]pyridazine

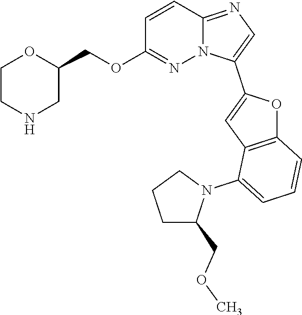 [SMILES: COC[C@H]1CCCN1c1cccc2oc(-c3cnc4ccc(OC[C@H]5CNCCO5)nn34)cc12]

To 100 mg (0.32 mmol) 3-bromo-6-[(2R)-morpholin-2-ylmethoxy]imidazo-[1,2-b]pyridazine were added 244 mg (calculated as 175.7 mg pure material) (0.64 mmol) {4-[(2R)-2-(methoxymethyl)pyrrolidin-1-yl]-1-benzofuran-2-yl}boronic acid dissolved in 4.6 mL 1-propanol, 0.48 mL (0.96 mmol) 2M aqueous potassiumcarbonate solution, 8.3 mg (0.03 mmol) triphenylphosphine and 22.5 mg (0.03 mmol) PdCl₂(PPh₃)₂. It was stirred 2 h at 130° C. bath temperature. The reaction mixture was cooled to room temperature and diluted with dichloromethane. The organic phase was washed with water, dried over magnesium sulfate and concentrated. The residue was purified by HPLC to give 77 mg (52%) of the title compound as solid material.

¹H-NMR (400 MHz, DMSO-d₆), δ [ppm]=1.90-2.10 (4H), 2.50-2.58 (1H), 2.59-2.71 (2H), 2.84-2.92 (1H), 3.19-3.25 (5H), 3.42-3.53 (3H), 3.68-3.85 (3H), 4.21-4.29 (1H), 4.34-4.44 (2H), 6.37 (1H), 6.87 (1H), 7.02 (1H), 7.12 (1H), 7.84-7.88 (1H), 8.06 (1H), 8.13 (1H).

LC-MS (Method 6): Rₜ=1.03 min; MS (ESIpos) m/z=464 [M+H]⁺.

### Example II-8

### (2S)-1-[(3-{4-[(2R)-2-(Methoxymethyl)pyrrolidin-1-yl]-1-benzofuran-2-yl}-imidazo[1,2-b]pyridazin-6-yl)oxy]propan-2-amine

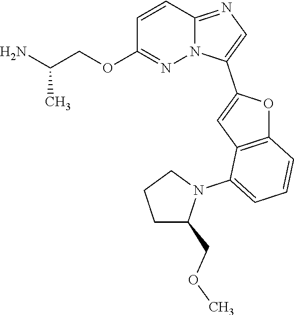 [SMILES: COC[C@H]1CCCN1c1cccc2oc(-c3cnc4ccc(OC[C@H](C)N)nn34)cc12]

121 mg (0.5 mmol) (2S)-1-[(3-bromoimidazo[1,2-b]pyridazin-6-yl)oxy]propan-2-amine, 340 mg (0.8 mmol) {4-[(2R)-2-(methoxymethyl)pyrrolidin-1-yl]-1-benzofuran-2-yl}boronic acid (approximately 65% pure), 12 mg (47 μmol) triphenylphosphine, 26 mg (47 μmol) Pddba₂ and 0.67 mL (1.3 mmol) potassium carbonate (c=2 mol/L in water) in 5.2 mL n-propanol were heated to reflux for 2 h.

The mixture was poured into water and extracted with dichloromethane. The organic layer was dried over sodium sulfate and evaporated. The residue was purified by HPLC to give 93 mg of the title compound as solid material.

¹H-NMR (300 MHz, DMSO-d₆), δ [ppm]=1.91-2.06 (2H), 2.19-2.32 (2H), 3.01 (3H), 3.18-3.26 (4H), 3.38-3.53 (2H), 3.70 (1H), 4.24 (1H), 4.48-4.65 (2H), 6.35 (1H), 6.86 (1H), 6.98 (1H), 7.07-7.16 (1H), 7.82 (1H), 8.07 (1H), 8.14 (1H).

LC-MS (Method 7): Rₜ=0.95 min; MS (ESIpos) m/z=422 [M+H]⁺.

### Example II-9

### (5R)-5-{[(3-{4-[(2R)-2-(Methoxymethyl)pyrrolidin-1-yl]-1-benzofuran-2-yl}imidazo[1,2-b]pyridazin-6-yl)oxy]methyl}pyrrolidin-2-one

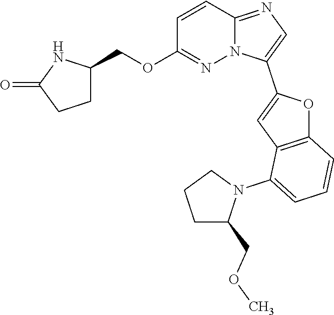 [SMILES: COC[C@H]1CCCN1c1cccc2oc(-c3cnc4ccc(OC[C@H]5CCC(=O)N5)nn34)cc12]

147 mg (0.47 mmol) (5R)-5-{[(3-bromoimidazo[1,2-b]pyridazin-6-yl)oxy]methyl}pyrrolidin-2-one, 340 mg (0.8 mmol) {4-[(2R)-2-(methoxymethyl)pyrrolidin-1-yl]-1-benzofuran-2-yl}boronic acid (approximately 65% pure), 12 mg (47 μmol) triphenylphosphine, 27 mg (47 μmol) Pddba₂ and 0.71 mL (1.3 mmol) potassium carbonate (c=2 mol/L in water) in 5.5 mL n-propanol were heated to reflux for 2 h.

The mixture was poured into water and extracted with dichloromethane. The organic layer was dried over sodium sulfate and evaporated. The residue was purified by HPLC to give 94 mg of the title compound as solid material.

¹H-NMR (300 MHz, DMSO-d₆), δ [ppm]=1.77-2.10 (5H), 2.10-2.35 (4H), 3.18-3.23 (4H), 3.40-3.53 (2H), 3.71 (1H), 4.00 (1H), 4.20-4.51 (3H), 6.36 (1H), 6.86 (1H), 6.97 (1H), 7.12 (1H), 7.83-7.95 (2H), 8.06 (1H), 8.14 (1H).

LC-MS (Method 7): Rₜ=1.21 min; MS (ESIpos) m/z=462 [M+H]⁺.

### Example II-10

### 6-Methoxy-3-{4-[(2R)-2-(methoxymethyl)pyrrolidin-1-yl]-1-benzofuran-2-yl}-imidazo[1,2-b]pyridazine

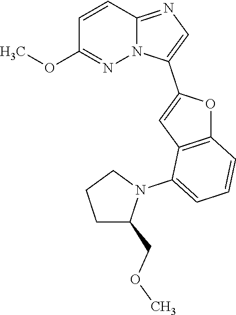 [SMILES: COC[C@H]1CCCN1c1cccc2oc(-c3cnc4ccc(OC)nn34)cc12]

108 mg (0.47 mmol) 3-bromo-6-methoxyimidazo[1,2-b]pyridazine, 340 mg (0.8 mmol) {4-[(2R)-2-(methoxymethyl)pyrrolidin-1-yl]-1-benzofuran-2-yl}boronic acid (approximately 65% pure), 12 mg (47 μmol) triphenylphosphine, 27 mg (47 μmol) Pddba₂ and 0.71 mL (1.3 mmol) potassium carbonate (c=2 mol/L in water) in 5.5 mL n-propanol were heated to reflux for 2 h.

The mixture was poured into water and extracted with dichloromethane. The organic layer was dried over sodium sulfate and evaporated. The residue was purified by HPLC to give 34 mg of the title compound as solid material.

¹H-NMR (300 MHz, DMSO-d₆), δ [ppm]=1.88-2.11 (4H), 3.18-3.24 (4H), 3.39-3.56 (2H), 3.67 (1H), 4.09 (3H), 4.25 (1H), 6.36 (1H), 6.87 (1H), 6.99 (1H), 7.12 (1H), 7.85 (1H), 8.06 (1H), 8.12 (1H).

LC-MS (Method 7): Rₜ=1.42 min; MS (ESIpos) m/z=379 [M+H]⁺.

### Example II-11

### trans-3-[(3-{4-[(2R)-2-(Methoxymethyl)pyrrolidin-1-yl]-1-benzofuran-2-yl}imidazo[1,2-b]pyridazin-6-yl)oxy]cyclobutanamine

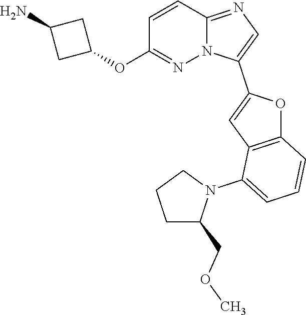 [SMILES: COC[C@H]1CCCN1c1cccc2oc(-c3cnc4ccc(O[C@H]5C[C@H](N)C5)nn34)cc12]

191 mg (0.5 mmol) tert-butyl {trans-3-[(3-bromoimidazo[1,2-b]pyridazin-6-yl)oxy]cyclobutyl}-carbamat, 360 mg (0.85 mmol) {4-[(2R)-2-(methoxymethyl)pyrrolidin-1-yl]-1-benzofuran-2-yl}boronic acid (approximately 65% pure), 13 mg (50 μmol) triphenylphosphine, 29 mg (50 μmol) Pddba₂ and 0.75 mL (1.5 mmol) potassium carbonate (c=2 mol/L in water) in 5.8 mL n-propanol were heated to reflux for 2 h.

The mixture was poured into water and extracted with dichloromethane. The organic layer was dried over sodium sulfate and evaporated.

The obtained crude product was suspended in 11 mL dichlormethane and 5 mL (65 mmol) TFA were added. The mixture was stirred for 20 min at room temperature.

5 mL ammonia (26% in water) were added. The mixture was extracted with dichloromethane. The organic layer was dried over sodium sulfate and evaporated. The residue was purified by HPLC to yield 22 mg of the title compound as solid material.

¹H-NMR (300 MHz, DMSO-d₆), δ [ppm]=1.93-2.14 (4H), 3.25 (4H), 3.51 (2H), 3.73-3.87 (2H), 4.20 (1H), 5.45 (1H), 6.40 (1H), 6.88 (1H), 6.97 (1H), 7.09-7.18 (1H), 7.67 (1H), 8.06 (1H), 8.13 (1H).

LC-MS (Method 7): Rₜ=1.34 min; MS (ESIpos) m/z=460 [M+H]⁺.

### Example III-12

### 6-[3-(Methylsulfonyl)propoxy]-3-[4-(4-phenylpiperazin-1-yl)-1-benzofuran-2-yl]-imidazo[1,2-b]pyridazine

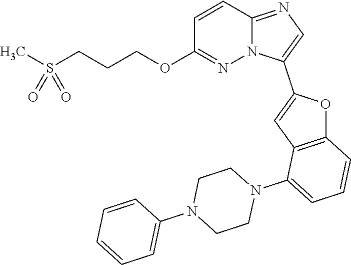 [SMILES: CS(=O)(=O)CCCOc1ccc2ncc(-c3cc4c(N5CCN(c6ccccc6)CC5)cccc4o3)n2n1]

167 mg (0.5 mmol) 3-bromo-6-[3-(methylsulfonyl)propoxy]imidazo[1,2-b]pyridazine, 447 mg (1.0 mmol) [4-(4-phenylpiperazin-1-yl)-1-benzofuran-2-yl]boronic acid (approximately 72% pure), 23 mg (20 μmol) Pd(PPh₃)₄, and 0.75 mL (1.5 mmol) potassium carbonate (c=2 mol/L in water) in 6 mL n-propanol were heated to reflux for 2 h.

The mixture was poured into water and extracted with dichloromethane. The organic layer was dried over sodium sulfate and evaporated.

The residue was digested two times with a 1:1 mixture of dichloromethane and methanol to yield 21 mg of the title compound as solid material.

¹H-NMR (300 MHz, DMSO-d₆), δ [ppm]=2.21-2.37 (3H), 2.93 (3H), 3.32-3.44 (10H), 4.65 (2H), 6.73-6.83 (2H), 6.97-7.07 (3H), 7.18-7.28 (4H), 7.65 (1H), 8.13-8.21 (2H).

LCMS (Method 7): Rₜ=1.36 min; MS (ESIpos) m/z=532 [M+H]⁺.

### Example III-13

### 3-[4-(Morpholin-4-yl)-1-benzofuran-2-yl]-6-[(3R)-pyrrolidin-3-yloxy]imidazo[1,2-b]pyridazine

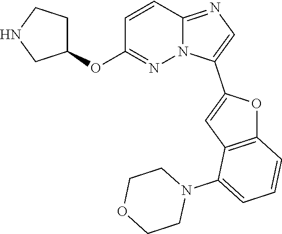 [SMILES: c1cc(N2CCOCC2)c2cc(-c3cnc4ccc(O[C@@H]5CCNC5)nn34)oc2c1]

To 150 mg (0.53 mmol) 3-bromo-6-[(3R)-pyrrolidin-3-yloxy]imidazo[1,2-b]pyridazine were added 439 mg (calculated as 261.8 mg pure material) (1.06 mmol) [4-(morpholin-4-yl)-1-benzofuran-2-yl]boronic acid dissolved in 7.7 mL 1-propanol, 0.80 mL (1.60 mmol) 2M aqueous potassiumcarbonate solution, 14 mg (0.05 mmol) triphenylphosphine and 37 mg (0.05 mmol) PdCl₂(PPh₃)₂. It was stirred 2 h at 130° C. bath temperature. The reaction mixture was cooled to room temperature and diluted with dichloromethane. The organic phase was washed with water, dried over magnesium sulfate and concentrated. The residue was purified by HPLC to give 15.5 mg (7%) of the title compound as solid material.

¹H-NMR (500 MHz, DMSO-d₆), δ [ppm]=2.06-2.25 (2H), 2.94-3.12 (2H), 3.17-3.22 (4H), 3.80-3.88 (4H), 5.52-5.58 (1H), 6.74-6.79 (1H), 6.98-7.02 (1H), 7.24-7.29 (2H), 7.59 (1H), 8.16 (2H).

LC-MS (Method 6): Rₜ=0.84 min; MS (ESIpos) m/z=406 [M+H]⁺.

### Example II-14

### (5R)-5-[({3-[4-(4-Phenylpiperazin-1-yl)-1-benzofuran-2-yl]imidazo[1,2-b]-pyridazin-6-yl}oxy)methyl]pyrrolidin-2-one

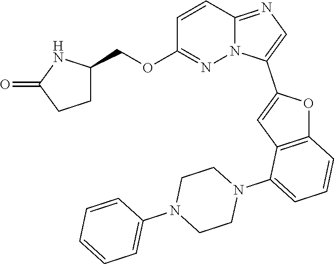 [SMILES: O=C1CC[C@H](COc2ccc3ncc(-c4cc5c(N6CCN(c7ccccc7)CC6)cccc5o4)n3n2)N1]

156 mg (0.5 mmol) (5R)-5-{[(3-bromoimidazo[1,2-b]pyridazin-6-yl)oxy]methyl}pyrrolidin-2-one, 447 mg (1.0 mmol) [4-(4-phenylpiperazin-1-yl)-1-benzofuran-2-yl]boronic acid (approximately 72% pure), 23 mg (20 μmol) Pd(PPh₃)₄, and 0.75 mL (1.5 mmol) potassium carbonate (c=2 mol/L in water) in 6 mL n-propanol were heated to reflux for 2 h.

The mixture was poured into water and extracted with dichloromethane. The organic layer was dried over sodium sulfate and evaporated.

The residue was purified by HPLC to yield 27 mg of the title compound as solid material.

¹H-NMR (300 MHz, DMSO-d₆), δ [ppm]=1.79-1.93 (1H), 2.03-2.30 (3H), 3.30-3.42 (8H), 3.99 (1H), 4.37 (1H), 4.53 (1H), 6.74-6.82 (3H), 6.95-7.05 (5H), 7.15-7.27 (6H), 7.63 (1H), 7.94 (1H), 8.13-8.21 (2H).

LC-MS (Method 7): Rₜ=1.30 min; MS (ESIpos) m/z=509 [M+H]⁺.

### Example II-15

### (2S)-1-({3-[4-(4-Phenylpiperazin-1-yl)-1-benzofuran-2-yl]imidazo[1,2-b]-pyridazin-6-yl}oxy)propan-2-amine

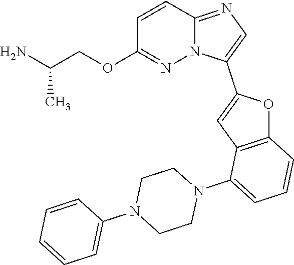 [SMILES: C[C@H](N)COc1ccc2ncc(-c3cc4c(N5CCN(c6ccccc6)CC5)cccc4o3)n2n1]

136 mg (0.5 mmol) (2S)-1-[(3-bromoimidazo[1,2-b]pyridazin-6-yl)oxy]propan-2-amine, 447 mg (1.0 mmol) [4-(4-phenylpiperazin-1-yl)-1-benzofuran-2-yl]boronic acid (approximately 72% pure), 23 mg (20 μmol) Pd(PPh₃)₄, and 0.75 mL (1.5 mmol) potassium carbonate (c=2 mol/L in water) in 6 mL n-propanol were heated to reflux for 2 h.

The mixture was poured into water and extracted with dichloromethane. The organic layer was dried over sodium sulfate and evaporated.

The residue was purified by HPLC to yield 61 mg of the title compound as solid material.

¹H-NMR (300 MHz, DMSO-d₆), δ [ppm]=1.08-1.17 (3H), 3.37 (8H), 4.20-4.33 (2H), 6.73-6.83 (2H), 6.94-7.07 (3H), 7.17-7.28 (4H), 7.64 (1H), 8.12-8.19 (2H).

LCMS (Method 7): Rₜ=1.00 min; MS (ESIpos) m/z=469 [M+H]⁺.

### Example II-16

### trans-3-({3-[4-(4-Phenyl piperazin-1-yl)-1-benzofuran-2-yl]imidazo[1,2-b]-pyridazin-6-yl}oxy)cyclobutanamine

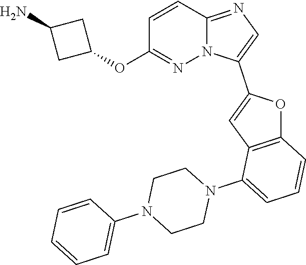 [SMILES: N[C@H]1C[C@H](Oc2ccc3ncc(-c4cc5c(N6CCN(c7ccccc7)CC6)cccc5o4)n3n2)C1]

192 mg (0.5 mmol) tert-Butyl {trans-3-[(3-bromoimidazo[1,2-b]pyridazin-6-yl)-oxy]cyclobutyl}-carbamate, 447 mg (1.0 mmol) [4-(4-phenylpiperazin-1-yl)-1-benzofuran-2-yl]boronic acid (approximately 72% pure), 23 mg (20 μmol) Pd(PPh₃)₄, and 0.75 mL (1.5 mmol) potassium carbonate (c=2 mol/L in water) in 6 mL n-propanol were heated to reflux for 2 h.

The mixture was poured into water and extracted with dichloromethane. The organic layer was dried over sodium sulfate and evaporated.

The obtained crude product was suspended in 10 mL dichlormethane and 5 mL (65 mmol) TFA were added. The mixture was stirred for 20 min at room temperature.

5 mL ammonia (26% in water) were added. The mixture was extracted with dichloromethane. The organic layer was dried over sodium sulfate and evaporated. The residue was purified by HPLC to yield 49 mg of the title compound as solid material.

¹H-NMR (300 MHz, DMSO-d₆), δ [ppm]=2.35 (4H), 3.66-3.81 (1H), 5.38 (1H), 6.72-6.84 (2H), 6.95-7.09 (3H), 7.17-7.28 (4H), 7.53 (1H), 8.08-8.26 (2H).

LC-MS (Method 7): Rₜ=0.98 min; MS (ESIpos) m/z=481[M+H]⁺.

### Example II-17

### (5R)-5-{[(3-{4-[Ethyl(2-methoxyethyl)amino]-1-benzofuran-2-yl}imidazo[1,2-b]-pyridazin-6-yl)oxy]methyl}pyrrolidin-2-one

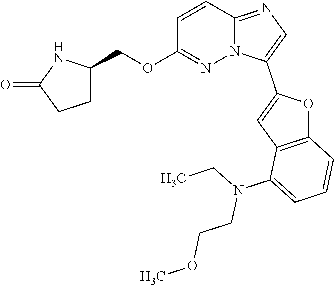 [SMILES: CCN(CCOC)c1cccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (100 mg) in 1-propanol (9 ml) was added 2M potassium carbonate solution (0.5 ml), crude {4-[ethyl(2-methoxyethyl)amino]-1-benzofuran-2-yl}boronic acid (calculated purity 68%) (248 mg), triphenylphosphine (8.4 mg) and PdCl₂(PPh₃)₂ (22.5 mg). The mixture was heated to reflux for 1 h. The hot mixture was filtered, the solvent was removed in vacuum, a half-saturated solution of sodium bicarbonate was added and the mixture was extracted with a mixture of dichloromethane and methanol (10:1). The organic phase was dried (sodium sulfate), filtered and the solvent was removed in vacuum. Silicagel chromatography followed by preparative reverse phase HPLC gave 89 mg of the title compound.

¹H-NMR (400 MHz, DMSO-d₆), δ [ppm]=1.18 (3H), 1.80-1.96 (1H), 2.08-2.34 (3H), 3.24 (3H), 3.46-3.63 (6H), 3.99 (1H), 4.33 (1H), 4.47 (1H), 6.54 (1H), 6.92-7.04 (2H), 7.08-7.17 (1H), 7.65 (1H), 7.93 (1H), 8.09 (1H), 8.16 (1H).

LCMS (Method 9): Rₜ=1.10 min; MS (ESIpos) m/z=450 [M+H]⁺.

### Example II-18

### (5R)-5-[({3-[4-(4-Methylpiperazin-1-yl)-1-benzofuran-2-yl]imidazo[1,2-b]-pyridazin-6-yl}oxy)methyl]pyrrolidin-2-one

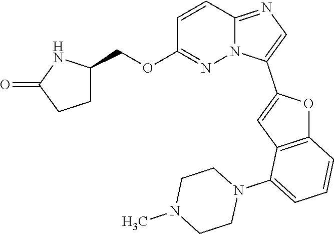 [SMILES: CN1CCN(c2cccc3oc(-c4cnc5ccc(OC[C@H]6CCC(=O)N6)nn45)cc23)CC1]

To 150 mg (0.48 mmol) (5R)-5-{[(3-bromoimidazo[1,2-b]pyridazin-6-yl)oxy]methyl}pyrrolidin-2-one were added 533 mg (calculated as 250.8 mg pure material) (0.96 mmol) [4-(4-methylpiperazin-1-yl)-1-benzofuran-2-yl]boronic acid dissolved in 7 mL 1-propanol, 0.72 mL (1.44 mmol) 2M aqueous potassiumcarbonate solution, 12.6 mg (0.05 mmol) triphenylphosphine and 34 mg (0.05 mmol) PdCl₂(PPh₃)₂. It was stirred 2 h at 130° C. bath temperature. The reaction mixture was cooled to room temperature and diluted with dichloromethane. The organic phase was washed with water, dried over magnesium sulfate and concentrated. The residue was purified by HPLC to give 5 mg (2%) of the title compound as solid material.

¹H-NMR (300 MHz, DMSO-d₆), δ [ppm]=1.85-2.00 (1H), 2.10-2.36 (6H), 2.51-2.58 (4H), 3.14-3.22 (4H), 3.96-4.06 (1H), 4.31-4.39 (1H), 4.44-4.51 (1H), 6.66-6.74 (1H), 7.02 (1H), 7.17-7.24 (2H), 7.59 (1H), 7.90 (1H), 8.13 (1H), 8.17 (1H).

LC-MS (Method 9): Rₜ=1.06 min; MS (ESIpos) m/z=446 [M+H]⁺.

### Example II-19

### (5R)-5-[({3-[4-(Piperazin-1-yl)-1-benzofuran-2-yl]imidazo[1,2-b]pyridazin-6-yl}-oxy)methyl]pyrrolidin-2-one

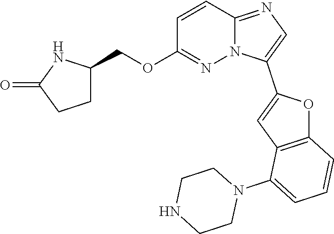 [SMILES: O=C1CC[C@H](COc2ccc3ncc(-c4cc5c(N6CCNCC6)cccc5o4)n3n2)N1]

114 mg (0.37 mmol) (5R)-5-{[(3-bromoimidazo[1,2-b]pyridazin-6-yl)oxy]methyl}-pyrrolidin-2-one, 638 mg (0.55 mmol) {4-[4-(tert-butoxycarbonyl)piperazin-1-yl]-1-benzofuran-2-yl}boronic acid (approximately 30% pure), 17 mg (15 μmol) Pd(PPh₃)₄, and 0.55 mL (1.1 mmol) potassium carbonate (c=2 mol/L in water) in 6 mL n-propanol were heated to reflux for 20 h.

The mixture was poured into water and extracted with dichloromethane. The organic layer was dried over sodium sulfate and evaporated.

The obtained crude product was suspended in 10 mL dichlormethane and 5 mL (65 mmol) TFA were added. The mixture was stirred for 15 min at room temperature.

5 mL ammonia (26% in water) were added. The mixture was extracted with dichloromethane. The organic layer was dried over sodium sulfate and evaporated. The residue was purified by HPLC to yield 21 mg of the title compound as solid material.

¹H-NMR (400 MHz, DMSO-d₆), δ [ppm]=1.86-1.96 (1H), 2.11-2.37 (3H), 3.03 (4H), 3.18 (4H), 4.01 (1H), 4.36 (1H), 4.50 (1H), 6.72 (1H), 7.03 (1H), 7.19-7.24 (2H), 7.59 (1H), 7.93 (1H), 8.12-8.15 (1H), 8.15-8.21 (2H).

LC-MS (Method 7): Rₜ=0.77 min; MS (ESIpos) m/z=433 [M+H]⁺.

### Example II-20

### 6-[3-(Methylsulfonyl)propoxy]-3-[4-(piperazin-1-yl)-1-benzofuran-2-yl]imidazo-[1,2-b]pyridazine

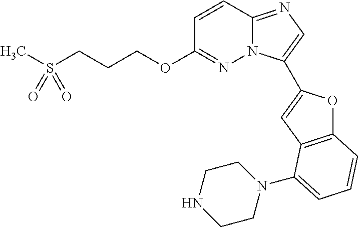 [SMILES: CS(=O)(=O)CCCOc1ccc2ncc(-c3cc4c(N5CCNCC5)cccc4o3)n2n1]

123 mg (0.37 mmol) 3-bromo-6-[3-(methylsulfonyl)propoxy]imidazo[1,2-b]pyridazine, 638 mg (0.55 mmol) {4-[4-(tert-butoxycarbonyl)piperazin-1-yl]-1-benzofuran-2-yl}boronic acid (approximately 30% pure), 17 mg (15 μmol) Pd(PPh₃)₄, and 0.55 mL (1.1 mmol) potassium carbonate (c=2 mol/L in water) in 6 mL n-propanol were heated to reflux for 20 h.

The mixture was poured into water and extracted with dichloromethane. The organic layer was dried over sodium sulfate and evaporated.

The obtained crude product was suspended in 10 mL dichlormethane and 5 mL (65 mmol) TFA were added. The mixture was stirred for 15 min at room temperature.

5 mL ammonia (26% in water) were added. The mixture was extracted with dichloromethane. The organic layer was dried over sodium sulfate and evaporated. The residue was purified by HPLC to yield 39 mg of the title compound as solid material.

¹H-NMR (400 MHz, DMSO-d₆), δ [ppm]=2.24-2.33 (2H), 2.99-3.07 (7H), 3.18 (4H), 3.32-3.38 (4H), 4.62 (2H), 6.69-6.75 (1H), 7.04 (1H), 7.19-7.24 (2H), 7.59 (1H), 8.13 (1H), 8.15-8.20 (2H).

LC-MS (Method 7): Rₜ=0.79 min; MS (ESIpos) m/z=456 [M+H]⁺.

### Example II-21

### 6-Methoxy-3-[4-(piperazin-1-yl)-1-benzofuran-2-yl]imidazo[1,2-b]pyridazine

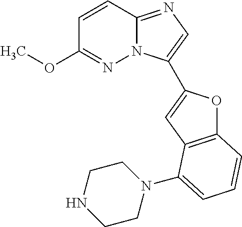 [SMILES: COc1ccc2ncc(-c3cc4c(N5CCNCC5)cccc4o3)n2n1]

84 mg (0.37 mmol) 3-bromo-6-methoxyimidazo[1,2-b]pyridazine, 638 mg (0.55 mmol) {4-[4-(tert-butoxycarbonyl)piperazin-1-yl]-1-benzofuran-2-yl}boronic acid (approximately 30% pure), 17 mg (15 μmol) Pd(PPh₃)₄, and 0.55 mL (1.1 mmol) potassium carbonate (c=2 mol/L in water) in 6 mL n-propanol were heated to reflux for 20 h.

The mixture was poured into water and extracted with dichloromethane. The organic layer was dried over sodium sulfate and evaporated.

The obtained crude product was suspended in 10 mL dichlormethane and 5 mL (65 mmol) TFA were added. The mixture was stirred for 15 min at room temperature.

5 mL ammonia (26% in water) were added. The mixture was extracted with dichloromethane. The organic layer was dried over sodium sulfate and evaporated. The residue was purified by HPLC to yield 29 mg of the title compound as solid material.

¹H-NMR (400 MHz, DMSO-d₆), δ [ppm]=3.02 (4H), 3.19 (4H), 4.12 (3H), 6.68-6.76 (1H), 7.04 (1H), 7.21 (2H), 7.66 (1H), 8.11-8.21 (3H).

LC-MS (Method 7): Rₜ=0.80 min; MS (ESIpos) m/z=350 [M+H]⁺.

### Example II-22

### 6-Methoxy-3-[4-(4-phenylpiperazin-1-yl)-1-benzofuran-2-yl]imidazo[1,2-b]-pyridazine

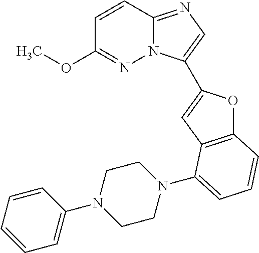 [SMILES: COc1ccc2ncc(-c3cc4c(N5CCN(c6ccccc6)CC5)cccc4o3)n2n1]

114 mg (0.5 mmol) 3-bromo-6-methoxyimidazo[1,2-b]pyridazine, 447 mg (1.0 mmol) [4-(4-phenylpiperazin-1-yl)-1-benzofuran-2-yl]boronic acid (approximately 72% pure), 23 mg (20 μmol) Pd(PPh₃)₄, and 0.75 mL (1.5 mmol) potassium carbonate (c=2 mol/L in water) in 6 mL n-propanol were heated to reflux for 2 h.

The mixture was poured into water and extracted with dichloromethane. The organic layer was dried over sodium sulfate and evaporated.

The residue was purified by HPLC followed by flash chromatography. The obtained material was digested in methanol to yield 8 mg of the title compound as solid material.

¹H-NMR (300 MHz, DMSO-d₆), δ [ppm]=3.35 (8H), 4.14 (3H), 6.74-6.84 (2H), 6.95-7.07 (3H), 7.18-7.28 (4H), 7.71 (1H), 8.12-8.19 (2H).

LC-MS (Method 8): Rₜ=1.55 min; MS (ESIpos) m/z=426 [M+H]⁺.

### Example II-23

### N-Ethyl-N-(2-methoxyethyl)-2-{6-[3-(methylsulfonyl)propoxy]imidazo[1,2-b]-pyridazin-3-yl}-1-benzofuran-4-amine

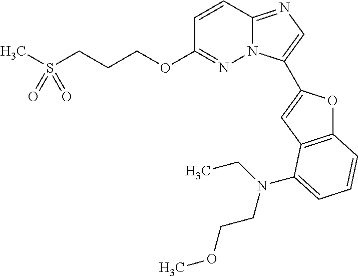 [SMILES: CCN(CCOC)c1cccc2oc(-c3cnc4ccc(OCCCS(C)(=O)=O)nn34)cc12]

To a stirred solution of 3-bromo-6-[3-(methylsulfonyl)propoxy]imidazo[1,2-b]pyridazine (150 mg) in 1-propanol (12 ml) was added 2M potassium carbonate solution (0.7 ml), crude {4-[ethyl(2-methoxyethyl)amino]-1-benzofuran-2-yl}boronic acid (calculated purity 68%) (346 mg), triphenylphosphine (11.8 mg) and PdCl₂(PPh₃)₂ (31.5 mg). The mixture was heated to reflux for 1 h. The hot mixture was filtered, the solvent was removed in vacuum, a half-saturated solution of sodium bicarbonate was added and the mixture was extracted with a mixture of dichloromethane and methanol (10:1). The organic phase was dried (sodium sulfate), filtered and the solvent was removed in vacuum. Silicagel chromatography followed by preparative reverse phase HPLC gave 96 mg of the title compound.

¹H-NMR (300 MHz, DMSO-d₆), δ [ppm]=1.17 (3H), 2.18-2.33 (2H), 3.01 (3H), 3.26 (3H), 3.34 (2H), 3.45-3.64 (6H), 4.57 (2H), 6.53 (1H), 6.92-7.03 (2H), 7.08-7.17 (1H), 7.63 (1H), 8.09 (1H), 8.15 (1H).

LCMS (Method 9): Rₜ=1.21 min; MS (ESIpos) m/z=473 [M+H]⁺.

### Example II-24

### 2-(6-{[(2S)-2-Aminopropyl]oxy}imidazo[1,2-b]pyridazin-3-yl)-N-ethyl-N-(2-methoxyethyl)-1-benzofuran-4-amine

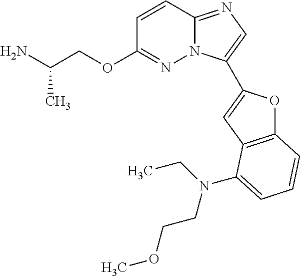 [SMILES: CCN(CCOC)c1cccc2oc(-c3cnc4ccc(OC[C@H](C)N)nn34)cc12]

To a stirred solution of (2S)-1-[(3-Bromoimidazo[1,2-b]pyridazin-6-yl)oxy]propan-2-amine (100 mg) in 1-propanol (10 ml) was added 2M potassium carbonate solution (0.6 ml), crude {4-[ethyl(2-methoxyethyl)amino]-1-benzofuran-2-yl}boronic acid (calculated purity 68%) (285 mg), triphenylphosphine (9.7 mg) and PdCl₂(PPh₃)₂ (25.9 mg). The mixture was heated to reflux for 1 h. The hot mixture was filtered, the solvent was removed in vacuum, a half-saturated solution of sodium bicarbonate was added and the mixture was extracted with a mixture of dichloromethane and methanol (10:1). The organic phase was dried (sodium sulfate), filtered and the solvent was removed in vacuum. Silicagel chromatography followed by preparative reverse phase HPLC gave 26 mg of the title compound.

¹H-NMR (400 MHz, DMSO-d₆, detected signals), δ [ppm]=1.10 (3H), 1.19 (3H), 1.76 (2H), 3.24 (3H), 3.48-3.61 (6H), 4.14-4.27 (2H), 6.55 (1H), 6.94-7.03 (2H), 7.10-7.16 (1H), 7.66 (1H), 8.07 (1H), 8.13 (1H).

LCMS (Method 9): Rₜ=1.26 min; MS (ESIpos) m/z=410 [M+H]⁺.

### Example II-25

### [{3-[(3-{4-[2-(Methoxymethyl)pyrrolidin-1-yl]-1-benzofuran-2-yl}imidazo-[1,2-b]pyridazin-6-yl)oxy]propyl}(methyl)oxido-λ⁶-sulfanylidene]cyanamide

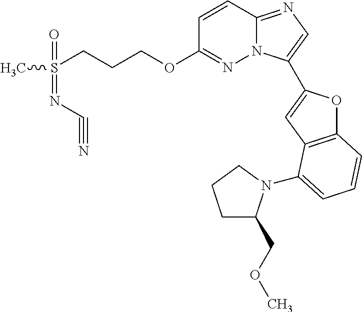 [SMILES: COC[C@H]1CCCN1c1cccc2oc(-c3cnc4ccc(OCCCS(C)(=O)=NC#N)nn34)cc12]

82 mg (0.23 mmol) [{3-[(3-bromoimidazo[1,2-b]pyridazin-6-yl)oxy]propyl}-(methyl)oxido-λ⁶-sulfanylidene]cyanamide, 100 mg (0.23 mmol) {4-[(2R)-2-(methoxymethyl)pyrrolidin-1-yl]-1-benzofuran-2-yl}boronic acid, 26 mg (23 μmol) Pd(PPh₃)₄, and 0.17 mL (0.34 mmol) potassium carbonate (c=2 mol/L in water) in 1.2 mL 1,4-dioxane were heated to reflux for 20 h.

Saturated aqueous ammonium chloride solution and ethyl acetate were added. The organic layer was separated (filtration via hydrophobic phase separation paper) and the solvent evaporated.

The residue was purified by HPLC to yield 9 mg of the title compound as solid material.

¹H-NMR (400 MHz, DMSO-d₆), δ [ppm]=1.92-2.11 (4H), 2.38 (2H), 3.22-3.25 (3H), 3.47 (1H), 3.51 (3H), 3.71 (1H), 3.79-3.87 (2H), 4.26 (1H), 4.54-4.70 (2H), 6.37 (1H), 6.87 (1H), 7.00 (1H), 7.13 (1H), 7.84 (1H), 8.07-8.09 (1H), 8.14-8.19 (1H).

LC-MS (Method 10): Rₜ=1.27 min; MS (ESIpos) m/z=509 [M+H]⁺.

### Example II-26

### (2R)-1-[(3-{4-[(2R)-2-(Methoxymethyl)pyrrolidin-1-yl]-1-benzofuran-2-yl}imidazo[1,2-b]pyridazin-6-yl)oxy]propan-2-amine

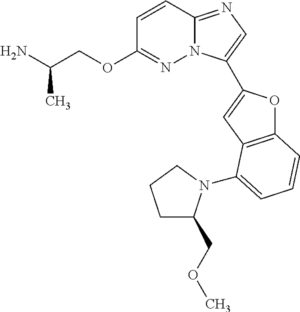 [SMILES: COC[C@H]1CCCN1c1cccc2oc(-c3cnc4ccc(OC[C@@H](C)N)nn34)cc12]

62 mg (0.23 mmol) (2R)-1-[(3-bromoimidazo[1,2-b]pyridazin-6-yl)oxy]propan-2-amine, 100 mg (0.23 mmol) {4-[(2R)-2-(methoxymethyl)pyrrolidin-1-yl]-1-benzofuran-2-yl}boronic acid, 26 mg (23 μmol) Pd(PPh₃)₄, and 0.17 mL (0.34 mmol) potassium carbonate (c=2 mol/L in water) in 1.2 mL 1,4-dioxane were heated to reflux for 20 h.

Saturated aqueous ammonium chloride solution and ethyl acetate were added. The organic layer was separated (filtration via hydrophobic phase separation paper) and the solvent evaporated.

The residue was purified by HPLC to yield 6 mg of the title compound as solid material.

¹H-NMR (300 MHz, DMSO-d₆), δ [ppm]=1.09 (3H), 1.69 (1H), 1.88-2.09 (4H), 3.18-3.26 (4H), 3.48 (2H), 3.69-3.80 (1H), 4.13-4.31 (3H), 6.37 (1H), 6.87 (1H), 7.00 (1H), 7.12 (1H), 7.90 (1H), 8.06 (1H), 8.13 (1H).

LC-MS (Method 10): Rₜ=1.32 min; MS (ESIpos) m/z=422 [M+H]⁺.

The following examples have been prepared in analogy to the examples above, using starting materials which were either commercially available or which have been prepared by methods described in the literature.

| | | | MW | Retention | |
|---|---|---|---|---|---|
| Exam- | | | found | time | HPLC |
| ple | Structure | Name | [M + H]⁺ | [min] | Method |
| II-27 | 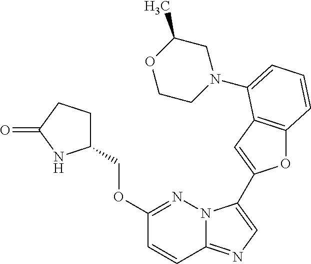 [SMILES: C[C@H]1CN(c2cccc3oc(-c4cnc5ccc(OC[C@H]6CCC(=O)N6)nn45)cc23)CCO1] | (5R)-5-{[(3-{4-[(2S)-2- methylmorpholin-4-yl]-1- benzofuran-2- yl}imidazo[1,2-b]pyridazin- 6-yl)oxy]methyl}pyrrolidin- 2-one | 448 | 1.1 | 7 |
| II-28 | 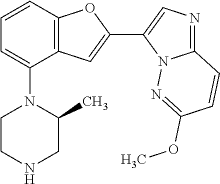 [SMILES: COc1ccc2ncc(-c3cc4c(N5CCNC[C@@H]5C)cccc4o3)n2n1] | 6-methoxy-3-{4-[(2S)-2- methylpiperazin-1-yl]-1- benzofuran-2- yl}imidazo[1,2-b]pyridazine | 364 | 0.83 | 7 |
| II-29 | 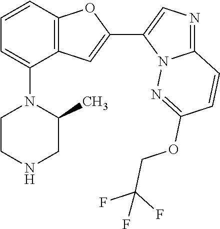 [SMILES: C[C@H]1CNCCN1c1cccc2oc(-c3cnc4ccc(OCC(F)(F)F)nn34)cc12] | 3-{4-[(2S)-2- methylpiperazin-1-yl]-1- benzofuran-2-yl}-6-(2,2,2- trifluoroethoxy)im- idazo[1,2-b]pyridazine | 432 | 0.91 | 7 |
| II-30 | 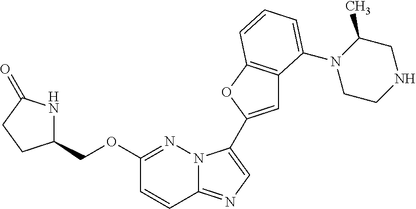 [SMILES: C[C@H]1CNCCN1c1cccc2oc(-c3cnc4ccc(OC[C@H]5CCC(=O)N5)nn34)cc12] | (5R)-5-{[(3-{4-[(2S)-2- methylpiperazin-1-yl]-1- benzofuran-2- yl}imidazo[1,2-b]pyridazin- 6-yl)oxy]methyl}pyrrolidin- 2-one | 447 | 0.78 | 7 |
| II-31 | 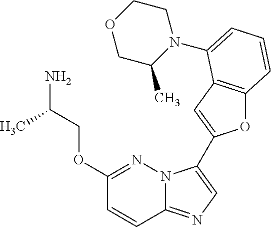 [SMILES: C[C@H](N)COc1ccc2ncc(-c3cc4c(N5CCOC[C@@H]5C)cccc4o3)n2n1] | (2S)-1-[(3-{4-[(3S)-3- methylmorpholin-4-yl]-1- benzofuran-2- yl}imidazo[1,2-b]pyridazin- 6-yl)oxy]propan-2-amine | 408 | 0.86 | 7 |
| II-32 | 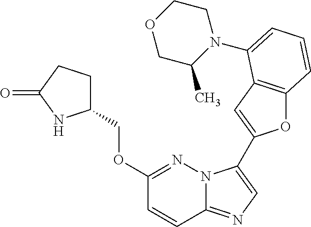 [SMILES: C[C@H]1COCCN1c1cccc2oc(-c3cnc4ccc(OC[C@H]5CCC(=O)N5)nn34)cc12] | (5R)-5-{[(3-{4-[(3S)-3- methylmorpholin-4-yl]-1- benzofuran-2- yl}imidazo[1,2-b]pyridazin- 6-yl)oxy]methyl}pyrrolidin- 2-one | 448 | 1.07 | 7 |
| II-33 | 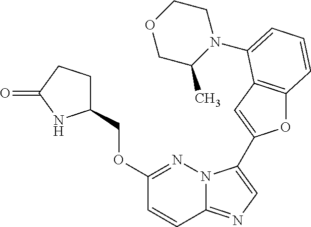 [SMILES: C[C@H]1COCCN1c1cccc2oc(-c3cnc4ccc(OC[C@@H]5CCC(=O)N5)nn34)cc12] | (5S)-5-{[(3-{4-[(3S)-3- methylmorpholin-4-yl]-1- benzofuran-2- yl}imidazo[1,2-b]pyridazin- 6-yl)oxy]methyl}pyrrolidin- 2-one | 448 | 1.07 | 7 |
| II-34 | 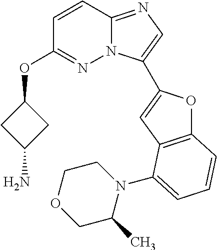 [SMILES: C[C@H]1COCCN1c1cccc2oc(-c3cnc4ccc(O[C@H]5C[C@H](N)C5)nn34)cc12] | trans-3-[(3-{4-[(3S)-3- methylmorpholin-4-yl]-1- benzofuran-2- yl}imidazo[1,2-b]pyridazin- 6-yl)oxy]cyclobutanamine | 420 | 0.84 | 7 |
| II-35 | 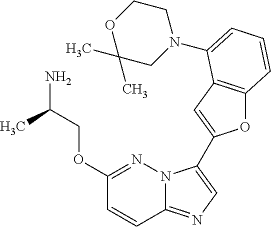 [SMILES: C[C@@H](N)COc1ccc2ncc(-c3cc4c(N5CCOC(C)(C)C5)cccc4o3)n2n1] | (2R)-1-({3-[4-(2,2- dimethylmorpholin-4-yl)- 1-benzofuran-2- yl]imidazo[1,2-b]pyridazin- 6-yl}oxy)propan-2-amine | 422 | 0.92 | 7 |
| II-36 | 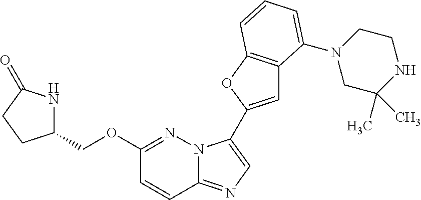 [SMILES: CC1(C)CN(c2cccc3oc(-c4cnc5ccc(OC[C@@H]6CCC(=O)N6)nn45)cc23)CCN1] | (5S)-5-[({3-[4-(3,3- dimethylpiperazin-1-yl)-1- benzofuran-2- yl]imidazo[1,2-b]pyridazin- 6-yl}oxy)methyl]pyrrolidin- 2-one | 461 | 0.77 | 6 |
| II-37 | 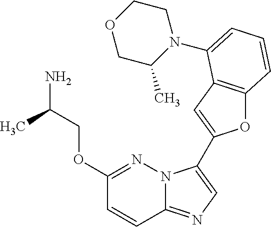 [SMILES: C[C@@H]1COCCN1c1cccc2oc(-c3cnc4ccc(OC[C@@H](C)N)nn34)cc12] | (2R)-1-[(3-{4-[(3R)-3- methylmorpholin-4-yl]-1- benzofuran-2- yl}imidazo[1,2-b]pyridazin- 6-yl)oxy]propan-2-amine | 408 | 0.92 | 7 |
| II-38 | 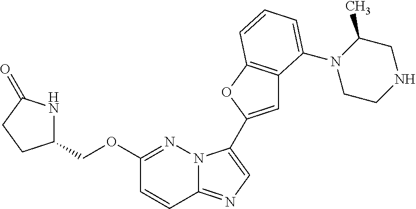 [SMILES: C[C@H]1CNCCN1c1cccc2oc(-c3cnc4ccc(OC[C@@H]5CCC(=O)N5)nn34)cc12] | (5S)-5-{[(3-{4-[(2S)-2- methylpiperazin-1-yl]-1- benzofuran-2- yl}imidazo[1,2-b]pyridazin- 6-yl)oxy]methyl}pyrrolidin- 2-one | 447 | 0.78 | 7 |
| II-39 | 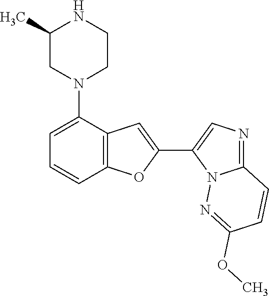 [SMILES: COc1ccc2ncc(-c3cc4c(N5CCN[C@H](C)C5)cccc4o3)n2n1] | 6-methoxy-3-{4-[(3R)-3- methylpiperazin-1-yl]-1- benzofuran-2- yl}imidazo[1,2-b]pyridazine | 364 | 0.77 | 6 |
| II-40 | 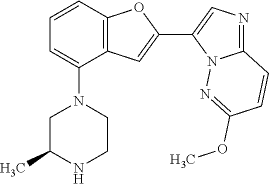 [SMILES: COc1ccc2ncc(-c3cc4c(N5CCN[C@@H](C)C5)cccc4o3)n2n1] | 6-methoxy-3-{4-[(3S)-3- methylpiperazin-1-yl]-1- benzofuran-2- yl}imidazo[1,2-b]pyridazine | 364 | 0.75 | 7 |
| II-41 | 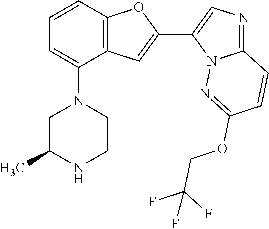 [SMILES: C[C@H]1CN(c2cccc3oc(-c4cnc5ccc(OCC(F)(F)F)nn45)cc23)CCN1] | 3-{4-[(3S)-3- methylpiperazin-1-yl]-1- benzofuran-2-yl}-6-(2,2,2- trifluoroethoxy)im- idazo[1,2-b]pyridazine | 432 | 0.87 | 7 |
| II-42 | 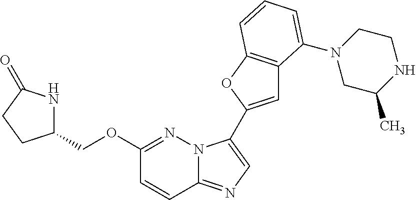 [SMILES: C[C@H]1CN(c2cccc3oc(-c4cnc5ccc(OC[C@@H]6CCC(=O)N6)nn45)cc23)CCN1] | (5S)-5-{[(3-{4-[(3S)-3- methylpiperazin-1-yl]-1- benzofuran-2- yl}imidazo[1,2-b]pyridazin- 6-yl)oxy]methyl}pyrrolidin- 2-one | 447 | 0.74 | 7 |
| II-43 | 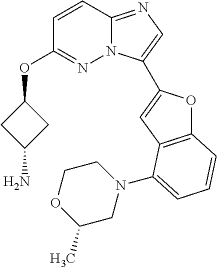 [SMILES: C[C@H]1CN(c2cccc3oc(-c4cnc5ccc(O[C@H]6C[C@H](N)C6)nn45)cc23)CCO1] | trans-3-[(3-{4-[(2S)-2- methylmorpholin-4-yl]-1- benzofuran-2- yl}imidazo[1,2-b]pyridazin- 6-yl)oxy]cyclobutanamine | 420 | 0.89 | 7 |
| II-44 | 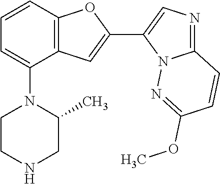 [SMILES: COc1ccc2ncc(-c3cc4c(N5CCNC[C@H]5C)cccc4o3)n2n1] | 6-methoxy-3-{4-[(2R)-2- methylpiperazin-1-yl]-1- benzofuran-2- yl}imidazo[1,2-b]pyridazine | 364 | 0.78 | 7 |
| II-45 | 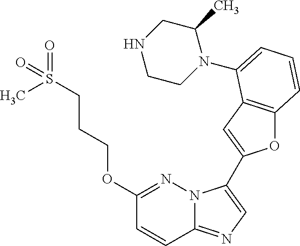 [SMILES: C[C@@H]1CNCCN1c1cccc2oc(-c3cnc4ccc(OCCCS(C)(=O)=O)nn34)cc12] | 3-{4-[(2R)-2- methylpiperazin-1-yl]-1- benzofuran-2-yl}-6-[3- (methylsulfonyl)pro- poxy]imidazo[1,2-b]pyri- dazine | 470 | 0.77 | 7 |
| II-46 | 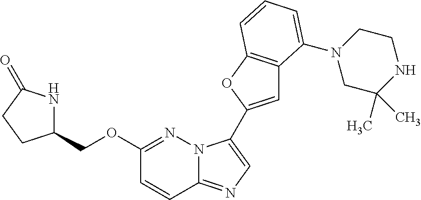 [SMILES: CC1(C)CN(c2cccc3oc(-c4cnc5ccc(OC[C@H]6CCC(=O)N6)nn45)cc23)CCN1] | (5R)-5-[({3-[4-(3,3- dimethylpiperazin-1-yl)-1- benzofuran-2- yl]imidazo[1,2-b]pyridazin- 6-yl}oxy)methyl]pyrrolidin- 2-one | 461 | 0.86 | 7 |
| II-47 | 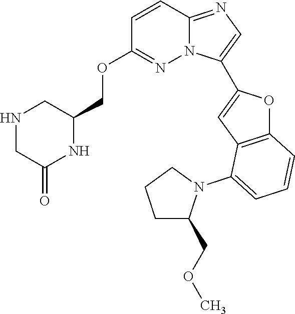 [SMILES: COC[C@H]1CCCN1c1cccc2oc(-c3cnc4ccc(OC[C@@H]5CNCC(=O)N5)nn34)cc12] | (6S)-6-{[(3-{4-[(2R)-2- (methoxymethyl)pyrrolidin- 1-yl]-1-benzofuran-2- yl}imidazo[1,2-b]pyridazin- 6-yl)oxy]methyl}piperazin- 2-one | 477 | 0.88 | 7 |
| II-48 | 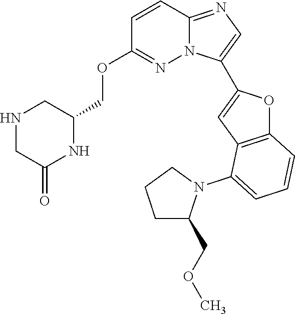 [SMILES: COC[C@H]1CCCN1c1cccc2oc(-c3cnc4ccc(OC[C@H]5CNCC(=O)N5)nn34)cc12] | (6R)-6-{[(3-{4-[(2R)-2- (methoxymethyl)pyrrolidin- 1-yl]-1-benzofuran-2- yl}imidazo[1,2-b]pyridazin- 6-yl)oxy]methyl}piperazin- 2-one | 477 | 0.88 | 7 |
| II-49 | 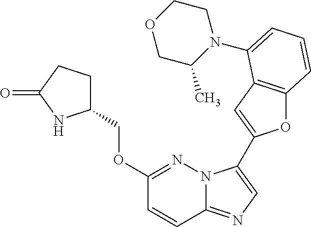 [SMILES: C[C@@H]1COCCN1c1cccc2oc(-c3cnc4ccc(OC[C@H]5CCC(=O)N5)nn34)cc12] | (5R)-5-{[(3-{4-[(3R)-3- methylmorpholin-4-yl]-1- benzofuran-2- yl}imidazo[1,2-b]pyridazin- 6-yl)oxy]methyl}pyrrolidin- 2-one | 448 | 1.06 | 6 |
| II-50 | 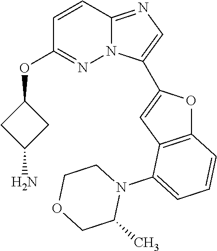 [SMILES: C[C@@H]1COCCN1c1cccc2oc(-c3cnc4ccc(O[C@H]5C[C@H](N)C5)nn34)cc12] | trans-3-[(3-{4-[(3R)-3- methylmorpholin-4-yl]-1- benzofuran-2- yl}imidazo[1,2-b]pyridazin- 6-yl)oxy]cyclobutanamine | 420 | 0.83 | 7 |
| II-51 | 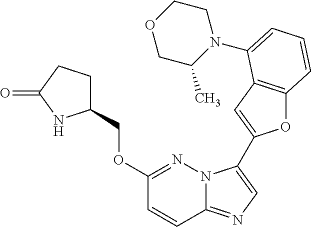 [SMILES: C[C@@H]1COCCN1c1cccc2oc(-c3cnc4ccc(OC[C@@H]5CCC(=O)N5)nn34)cc12] | (5S)-5-{[(3-{4-[(3R)-3- methylmorpholin-4-yl]-1- benzofuran-2- yl}imidazo[1,2-b]pyridazin- 6-yl)oxy]methyl}pyrrolidin- 2-one | 448 | 1.05 | 6 |
| II-52 | 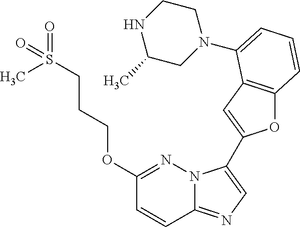 [SMILES: C[C@H]1CN(c2cccc3oc(-c4cnc5ccc(OCCCS(C)(=O)=O)nn45)cc23)CCN1] | 3-{4-[(3S)-3- methylpiperazin-1-yl]-1- benzofuran-2-yl}-6-[3- (methylsulfonyl)pro- poxy]imidazo[1,2-b]pyri- dazine | 470 | 0.78 | 7 |
| II-53 | 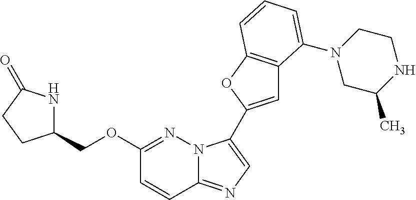 [SMILES: C[C@H]1CN(c2cccc3oc(-c4cnc5ccc(OC[C@H]6CCC(=O)N6)nn45)cc23)CCN1] | (5R)-5-{[(3-{4-[(3S)-3- methylpiperazin-1-yl]-1- benzofuran-2- yl}imidazo[1,2-b]pyridazin- 6-yl)oxy]methyl}pyrrolidin- 2-one | 447 | 0.76 | 7 |
| II-54 | 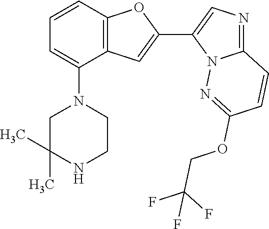 [SMILES: CC1(C)CN(c2cccc3oc(-c4cnc5ccc(OCC(F)(F)F)nn45)cc23)CCN1] | 3-[4-(3,3- dimethylpiperazin-1-yl)-1- benzofuran-2-yl]-6-(2,2,2- trifluoroethoxy)im- idazo[1,2-b]pyridazine | 446 | 0.93 | 6 |
| II-55 | 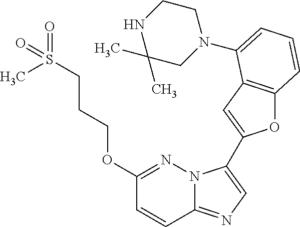 [SMILES: CC1(C)CN(c2cccc3oc(-c4cnc5ccc(OCCCS(C)(=O)=O)nn45)cc23)CCN1] | 3-[4-(3,3- dimethylpiperazin-1-yl)-1- benzofuran-2-yl]-6-[3- (methylsulfonyl)propoxy]im- idazo[l,2-b]pyridazine | 484 | 0.81 | 6 |
| II-56 | 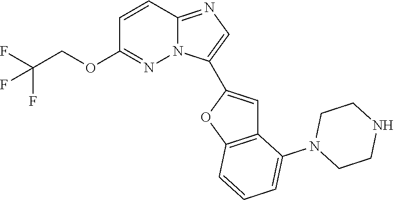 [SMILES: FC(F)(F)COc1ccc2ncc(-c3cc4c(N5CCNCC5)cccc4o3)n2n1] | 3-[4-(piperazin-1-yl)-1- benzofuran-2-yl]-6-(2,2,2- trifluoroethoxy)im- idazo[1,2-b]pyridazine | 418 | 1.18 | 10 |
| II-57 | 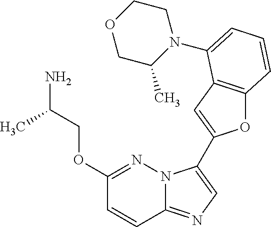 [SMILES: C[C@H](N)COc1ccc2ncc(-c3cc4c(N5CCOC[C@H]5C)cccc4o3)n2n1] | (2S)-1-[(3-{4-[(3R)-3- methylmorpholin-4-yl]-1- benzofuran-2- yl}imidazo[1,2-b]pyridazin- 6-yl)oxy]propan-2-amine | 408 | 0.92 | 7 |
| II-58 | 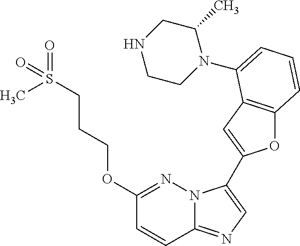 [SMILES: C[C@H]1CNCCN1c1cccc2oc(-c3cnc4ccc(OCCCS(C)(=O)=O)nn34)cc12] | 3-{4-[(2S)-2- methylpiperazin-1-yl]-1- benzofuran-2-yl}-6-[3- (methylsulfonyl)propoxy]im- idazo[1,2-b]pyridazine | 470 | 0.81 | 7 |
| II-59 | 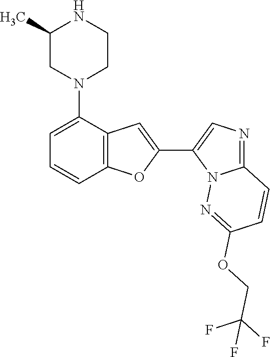 [SMILES: C[C@@H]1CN(c2cccc3oc(-c4cnc5ccc(OCC(F)(F)F)nn45)cc23)CCN1] | 3-{4-[(3R)-3- methylpiperazin-1-yl]-1- benzofuran-2-yl}-6-(2,2,2- trifluoroethoxy)im- idazo[1,2-b]pyridazine | 432 | 0.87 | 6 |
| II-60 | 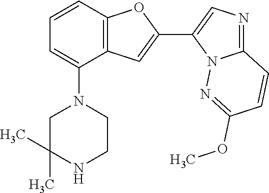 [SMILES: COc1ccc2ncc(-c3cc4c(N5CCNC(C)(C)C5)cccc4o3)n2n1] | 3-[4-(3,3- dimethylpiperazin-1-yl)-1- benzofuran-2-yl]-6-methoxy- imidazo[1,2-b]pyridazine | 378 | 0.8 | 7 |
| II-61 | 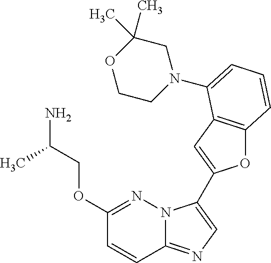 [SMILES: C[C@H](N)COc1ccc2ncc(-c3cc4c(N5CCOC(C)(C)C5)cccc4o3)n2n1] | (2S)-1-({3-[4-(2,2- dimethylmorpholin-4-yl)-1- benzofuran-2- yl]imidazo[1,2-b]pyridazin- 6-yl}oxy)propan-2-amine | 422 | 0.93 | 7 |
| II-62 | 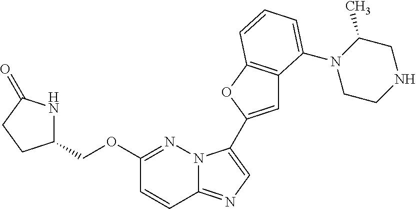 [SMILES: C[C@@H]1CNCCN1c1cccc2oc(-c3cnc4ccc(OC[C@@H]5CCC(=O)N5)nn34)cc12] | (5S)-5-{[(3-{4-[(2R)-2- methylpiperazin-1-yl]-1- benzofuran-2- yl}imidazo[1,2-b]pyridazin- 6-yl)oxy]methyl}pyrrolidin- 2-one | 447 | 0.8 | 7 |
| II-63 | 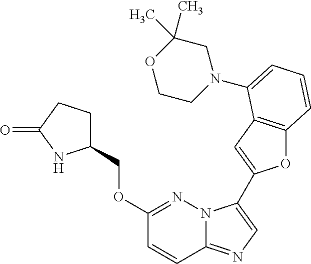 [SMILES: CC1(C)CN(c2cccc3oc(-c4cnc5ccc(OC[C@@H]6CCC(=O)N6)nn45)cc23)CCO1] | (5S)-5-[({3-[4-(2,2- dimethylmorpholin-4-yl)- 1-benzofuran-2- yl]imidazo[1,2-b]pyridazin- 6-yl}oxy)methyl]pyrrolidin- 2-one | 462 | 1.15 | 7 |
| II-64 | 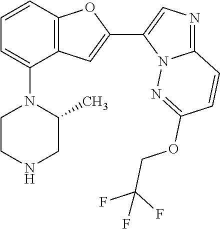 [SMILES: C[C@@H]1CNCCN1c1cccc2oc(-c3cnc4ccc(OCC(F)(F)F)nn34)cc12] | 3-{4-[(2R)-2- methylpiperazin-1-yl]-1- benzofuran-2-yl}-6-(2,2,2- trifluoroethoxy)im- idazo[1,2-b]pyridazine | 432 | 0.91 | 7 |
| II-65 | 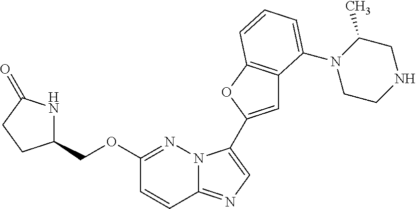 [SMILES: C[C@@H]1CNCCN1c1cccc2oc(-c3cnc4ccc(OC[C@H]5CCC(=O)N5)nn34)cc12] | (5R)-5-{[(3-{4-[(2R)-2- methylpiperazin-1-yl]-1- benzofuran-2- yl}imidazo[1,2-b]pyridazin- 6-yl)oxy]methyl}pyrrolidin- 2-one | 447 | 0.79 | 7 |
| II-66 | 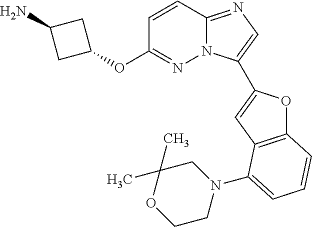 [SMILES: CC1(C)CN(c2cccc3oc(-c4cnc5ccc(O[C@H]6C[C@H](N)C6)nn45)cc23)CCO1] | trans-3-({3-[4-(2,2- dimethylmorpholin-4-yl)- 1-benzofuran-2- yl]imidazo[1,2-b]pyridazin- 6-yl}oxy)cyclobutanamine | 434 | 0.93 | 7 |
| II-67 | 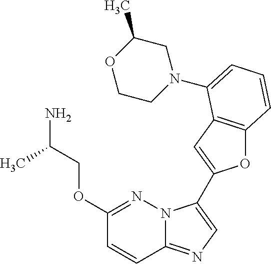 [SMILES: C[C@H](N)COc1ccc2ncc(-c3cc4c(N5CCO[C@@H](C)C5)cccc4o3)n2n1] | (2S)-1-[(3-{4-[(2S)-2- methylmorpholin-4-yl]-1- benzofuran-2- yl}imidazo[1,2-b]pyridazin- 6-yl)oxy]propan-2-amine | 408 | 0.9 | 7 |
| II-68 | 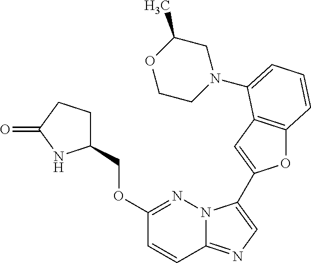 [SMILES: C[C@H]1CN(c2cccc3oc(-c4cnc5ccc(OC[C@@H]6CCC(=O)N6)nn45)cc23)CCO1] | (5S)-5-{[(3-{4-[(2S)-2- methylmorpholin-4-yl]-1- benzofuran-2- yl}imidazo[1,2-b]pyridazin- 6-yl)oxy]methyl}pyrrolidin- 2-one | 448 | 1.1 | 7 |
| II-69 | 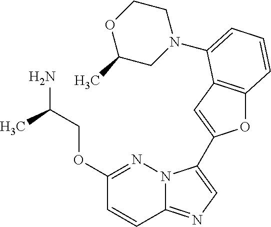 [SMILES: C[C@@H]1CN(c2cccc3oc(-c4cnc5ccc(OC[C@@H](C)N)nn45)cc23)CCO1] | (2R)-1-[(3-{4-[(2R)-2- methylmorpholin-4-yl]-1- benzofuran-2- yl}imidazo[1,2-b]pyridazin- 6-yl)oxy]propan-2-amine | 408 | 0.9 | 7 |
| II-70 | 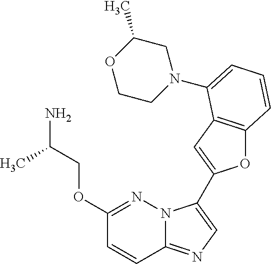 [SMILES: C[C@H](N)COc1ccc2ncc(-c3cc4c(N5CCO[C@H](C)C5)cccc4o3)n2n1] | (2S)-1-[(3-{4-[(2R)-2- methylmorpholin-4-yl]-1- benzofuran-2- yl}imidazo[1,2-b]pyridazin- 6-yl)oxy]propan-2-amine | 408 | 0.91 | 7 |
| II-71 | 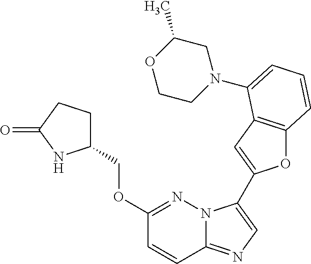 [SMILES: C[C@@H]1CN(c2cccc3oc(-c4cnc5ccc(OC[C@H]6CCC(=O)N6)nn45)cc23)CCO1] | (5R)-5-{[(3-{4-[(2R)-2- methylmorpholin-4-yl]-1- benzofuran-2- yl}imidazo[1,2-b]pyridazin- 6-yl)oxy]methyl}pyrrolidin- 2-one | 448 | 1.1 | 7 |
| II-72 | 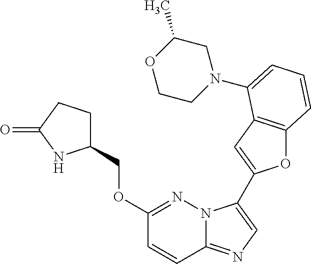 [SMILES: C[C@@H]1CN(c2cccc3oc(-c4cnc5ccc(OC[C@@H]6CCC(=O)N6)nn45)cc23)CCO1] | (5S)-5-{[(3-{4-[(2R)-2- methylmorpholin-4-yl]-1- benzofuran-2- yl}imidazo[1,2-b]pyridazin- 6-yl)oxy]methyl}pyrrolidin- 2-one | 448 | 1.1 | 7 |
| II-73 | 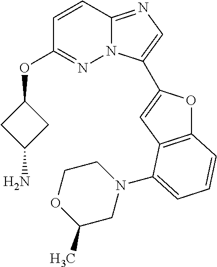 [SMILES: C[C@@H]1CN(c2cccc3oc(-c4cnc5ccc(O[C@H]6C[C@H](N)C6)nn45)cc23)CCO1] | trans-3-[(3-{4-[(2R)-2- methylmorpholin-4-yl]-1- benzofuran-2- yl}imidazo[1,2-b]pyridazin- 6-yl)oxy]cyclobutanamine | 420 | 0.9 | 7 |
| II-74 | 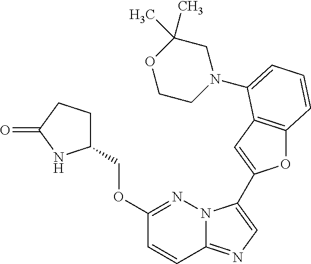 [SMILES: CC1(C)CN(c2cccc3oc(-c4cnc5ccc(OC[C@H]6CCC(=O)N6)nn45)cc23)CCO1] | (5R)-5-[({3-[4-(2,2- dimethylmorpholin-4-yl)- 1-benzofuran-2- yl]imidazo[1,2-b]pyridazin- 6-yl}oxy)methyl]pyrrolidin- 2-one | 462 | 1.15 | 7 |
| II-75 | 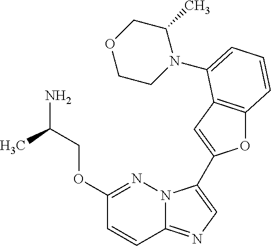 [SMILES: C[C@@H](N)COc1ccc2ncc(-c3cc4c(N5CCOC[C@@H]5C)cccc4o3)n2n1] | (2R)-1-[(3-{4-[(3S)-3- methylmorpholin-4-yl]-1- benzofuran-2- yl}imidazo[1,2-b]pyridazin- 6-yl)oxy]propan-2-amine | 408 | 0.86 | 7 |
| II-76 | 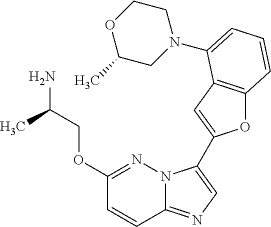 [SMILES: C[C@@H](N)COc1ccc2ncc(-c3cc4c(N5CCO[C@@H](C)C5)cccc4o3)n2n1] | (2R)-1-[(3-{4-[(2S)-2- methylmorpholin-4-yl]-1- benzofuran-2- yl}imidazo[1,2-b]pyridazin- 6-yl)oxy]propan-2-amine | 408 | 0.91 | 7 |

### Example III-001

### (2R)-1-({3-[4-(Morpholin-4-yl)furo[3,2-c]pyridin-2-yl]imidazo[1,2-b]pyridazin-6-yl}oxy)propan-2-amine

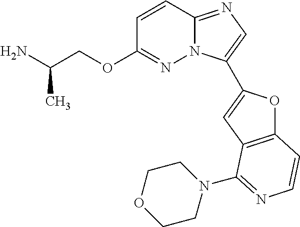 [SMILES: C[C@@H](N)COc1ccc2ncc(-c3cc4c(N5CCOCC5)nccc4o3)n2n1]

To a stirred solution of (2R)-1-[(3-bromoimidazo[1,2-b]pyridazin-6-yl)oxy]propan-2-amine (3.0 g) in 1-propanol (250 mL) was added 2M potassium carbonate solution (16.6 mL), crude [4-(morpholin-4-yl)furo[3,2-c]pyridin-2-yl]boronic acid (72% w/w; 4.19 g), triphenylphosphine (290 mg) and PdCl₂(PPh₃)₂ (777 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography followed by silicagel chromatography gave a solid that was triturated with ethanol to give 2.28 g of the title compound.

LCMS (Method 15): Rₜ=1.1.51 min; MS (ESIpos) m/z=606 [M+H]⁺.

¹H-NMR (400 MHz, DMSO-d₆), δ [ppm]=1.10 (3H), 1.64 (2H), 3.19-3.26 (1H), 3.61-3.67 (4H), 3.74-3.79 (4H), 4.11-4.20 (2H), 6.99 (1H), 7.09 (1H), 7.64 (1H), 8.01 (1H), 8.09 (1H), 8.12 (1H).

LC-MS (Method 15): Rₜ=0.97 min; MS (ESIpos) m/z=395 [M+H]⁺.

### Example III-002

### {1-[2-(6-{[(2R)-2-Aminopropyl]oxy}imidazo[1,2-b]pyridazin-3-yl)furo[3,2-c]pyridin-4-yl]piperidin-4-yl}methanol

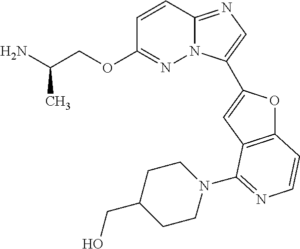 [SMILES: C[C@@H](N)COc1ccc2ncc(-c3cc4c(N5CCC(CO)CC5)nccc4o3)n2n1]

To a stirred solution of (2R)-1-[(3-{4-[4-({[tert-butyl(dimethyl)silyl]oxy}methyl) piperidin-1-yl]furo[3,2-c]pyridin-2-yl}imidazo[1,2-b]pyridazin-6-yl)oxy]propan-2-amine (75 mg) in THF (7.5 mL) was added a solution of TBAF in THF (0.35 mL; c=1.0 mol/L). The mixture was stirred at room temperature for 70 h. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with a mixture of ethyl acetate and hexane to give 25 mg of the title compound.

¹H-NMR (300 MHz, DMSO-d₆), δ [ppm]=1.10 (3H), 1.18-1.33 (2H), 1.66 (3H), 1.78 (2H), 3.00 (2H), 3.18-3.33 (3H), 4.09-4.26 (2H), 4.32-4.53 (3H), 6.97-7.06 (2H), 7.63 (1H), 7.96 (1H), 8.09 (1H), 8.13 (1H).

LC-MS; MS (ESIpos) m/z=423 [M+H]⁺.

### Example III-003

### (2R)-1-[(3-{4-[(3R)-3-Methylmorpholin-4-yl]furo[3,2-c]pyridin-2-yl}imidazo-[1,2-b]pyridazin-6-yl)oxy]propan-2-amine

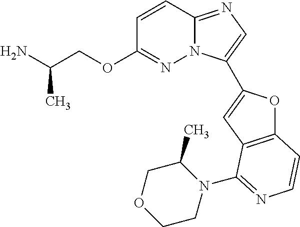 [SMILES: C[C@@H]1COCCN1c1nccc2oc(-c3cnc4ccc(OC[C@@H](C)N)nn34)cc12]

To 100 mg (0.37 mmol) (2R)-1-[(3-bromoimidazo[1,2-b]pyridazin-6-yl)oxy]propan-2-amine in 6 mL propan-1-ol were added 322 mg (0.74 mmol) {4-[(3R)-3-methylmorpholin-4-yl]furo[3,2-c]pyridin-2-yl}boronic acid, 17 mg (15 μmol) tetrakis(triphenylphosphin)palladium(0) and 0.55 mL of a 2 M aqueous solution of sodium carbonate. The mixture was stirred at 110° C. for 17 h.

The reaction mixture was poured into water and extracted with dichloromethane. The organic layer was dried over sodium sulfate, and concentrated. The crude product was purified by HPLC to give 63 mg of the title compound.

¹H-NMR (400 MHz, DMSO-d₆), δ [ppm]=1.11 (3H), 1.26 (3H), 3.20-3.25 (1H), 3.37 (1H), 3.56 (1H), 3.72 (2H), 3.95 (1H), 4.09-4.25 (3H), 4.53 (1H), 7.01-7.07 (2H), 7.70 (1H), 8.00 (1H), 8.10 (1H), 8.12-8.17 (1H).

LC-MS (Method 13): Rₜ=0.57 min; MS (ESIpos) m/z=409 [M+H]⁺.

### Example III-004

### 2-(6-{[(2R)-2-Aminopropyl]oxy}imidazo[1,2-b]pyridazin-3-yl)-N-ethyl-N-(2-methoxyethyl)furo[3,2-c]pyridin-4-amine

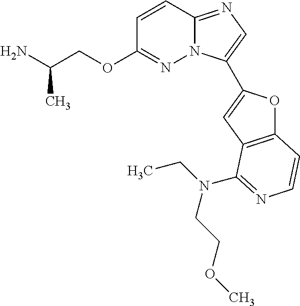 [SMILES: CCN(CCOC)c1nccc2oc(-c3cnc4ccc(OC[C@@H](C)N)nn34)cc12]

To 637 mg (2.3 mmol) (2R)-1-[(3-bromoimidazo[1,2-b]pyridazin-6-yl)oxy]propan-2-amine in 24 mL 1,4-dioxane were added 2 g (4.7 mmol) {4-[ethyl(2-methoxyethyl)amino]furo[3,2-c]pyridin-2-yl}boronic acid, 542 mg (0.47 mmol) tetrakis(triphenylphosphin)palladium(0) and 3.5 mL of a 2 M aqueous solution of sodium carbonate. The mixture was stirred at 100° C. for 17 h.

Saturated aqueous ammonium chloride solution was added. The mixture was extracted with ethyl acetate. The precipitate was filtered off. The precipitate was taken up in a mixture of methanol and dichloromethane. Aqueous ammonia was added until a basic pH was reached. The obtained solution was concentrated. Dichloromethane and water were added and the obtained mixture was shaken. The organic layer was separated, dried over sodium sulfate, and concentrated. The crude product was digested with methyl-tert.-butyl ether to give 607 mg of the title compound.

¹H-NMR (400 MHz, DMSO-d₆), δ [ppm]=1.12 (3H), 1.28 (3H), 3.28 (3H), 3.62 (2H), 3.78 (2H), 3.85 (2H), 4.15-4.25 (2H), 6.92 (1H), 7.03 (1H), 7.66 (1H), 7.95 (1H), 8.09 (1H), 8.15 (1H).

LC-MS (Method 16): Rₜ=1.16 min; MS (ESIpos) m/z=411 [M+H]⁺.

### Example III-005

### (2R)-1-[(3-{4-[(3S)-3-Methylmorpholin-4-yl]furo[3,2-c]pyridin-2-yl}imidazo-[1,2-b]pyridazin-6-yl)oxy]propan-2-amine

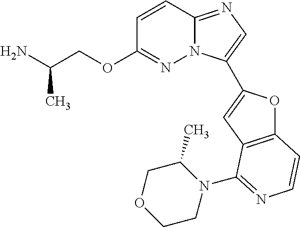 [SMILES: C[C@@H](N)COc1ccc2ncc(-c3cc4c(N5CCOC[C@@H]5C)nccc4o3)n2n1]

To 100 mg (0.37 mmol) (2R)-1-[(3-bromoimidazo[1,2-b]pyridazin-6-yl)oxy]propan-2-amine in 6 mL propan-1-ol were added 322 mg (0.74 mmol) {4-[(3S)-3-methylmorpholin-4-yl]furo[3,2-c]pyridin-2-yl}boronic acid, 17 mg (15 μmol) tetra-kis(triphenylphosphin)palladium(0) and 0.55 mL of a 2 M aqueous solution of sodium carbonate. The mixture was stirred at 110° C. for 17 h.

Half-saturated brine was added. The mixture was extracted with dichloromethane. The organic layer was separated, dried over sodium sulfate, and evaporated. The crude product was purified by HPLC to give 53 mg of the title compound.

¹H-NMR (300 MHz, DMSO-d₆), δ [ppm]=1.13 (3H), 1.29 (3H), 3.34-3.45 (1H), 3.53-3.65 (1H), 3.75 (2H), 3.93-4.02 (1H), 4.11-4.21 (2H), 4.22-4.32 (1H), 4.55 (1H), 7.02-7.11 (2H), 7.73 (1H), 8.03 (1H), 8.12-8.20 (2H).

LC-MS (Method 13): Rₜ=0.53 min; MS (ESIpos) m/z=409 [M+H]⁺.

### Example III-006

### (2R)-1-[(3-{4-[(2S)-2-Methylmorpholin-4-yl]furo[3,2-c]pyridin-2-yl}imidazo-[1,2-b]pyridazin-6-yl)oxy]propan-2-amine

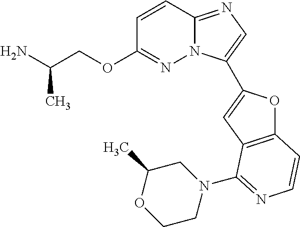 [SMILES: C[C@@H](N)COc1ccc2ncc(-c3cc4c(N5CCO[C@@H](C)C5)nccc4o3)n2n1]

To 100 mg (0.37 mmol) (2R)-1-[(3-bromoimidazo[1,2-b]pyridazin-6-yl)oxy]propan-2-amine in 6 mL propan-1-ol were added 311 mg (0.74 mmol) {4-[(2S)-2-methylmorpholin-4-yl]furo[3,2-c]pyridin-2-yl}boronic acid, 17 mg (15 mol) tetrakis(triphenylphosphin)palladium(0) and 0.55 mL of a 2 M aqueous solution of sodium carbonate. The mixture was stirred at 110° C. for 18 h.

Water was added. The mixture was extracted with dichloromethane. The organic layer was separated, dried over sodium sulfate, and evaporated. The crude product was purified by flash chromatography to give 118 mg of the title compound.

¹H-NMR (400 MHz, DMSO-d₆), δ [ppm]=1.17 (6H), 2.76 (1H), 3.09-3.21 (2H), 3.62-3.75 (2H), 3.91-4.00 (1H), 4.12-4.33 (4H), 7.05 (1H), 7.13 (1H), 7.68 (1H), 8.03 (1H), 8.13-8.21 (2H).

LC-MS (Method 14): Rₜ=0.57 min; MS (ESIpos) m/z=409 [M+H]⁺.

### Example III-007

### (2S)-1-({3-[4-(Morpholin-4-yl)furo[3,2-c]pyridin-2-yl]imidazo[1,2-b]pyridazin-6-yl}oxy)propan-2-amine

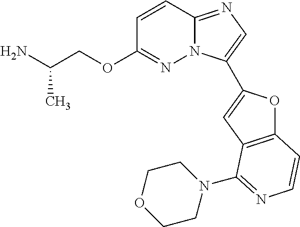 [SMILES: C[C@H](N)COc1ccc2ncc(-c3cc4c(N5CCOCC5)nccc4o3)n2n1]

To a stirred solution of (2S)-1-[(3-Bromoimidazo[1,2-b]pyridazin-6-yl)oxy]propan-2-amine (6.0 g) in 1-propanol (600 mL) was added 2M potassium carbonate solution (33.2 mL), crude [4-(morpholin-4-yl)furo[3,2-c]pyridin-2-yl]boronic acid (51% w/w; 21.5 g), triphenylphosphine (580 mg) and PdCl₂(PPh₃)₂ (1553 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography followed by silicagel chromatography gave a solid that was triturated with a mixture of dichloromethane and hexane to give 6.8 g of the title compound.

¹H-NMR (400 MHz, DMSO-d6), δ [ppm]=1.10 (3H), 1.64 (2H), 3.19-3.26 (1H), 3.60-3.68 (4H), 3.72-3.81 (4H), 4.10-4.21 (2H), 6.99 (1H), 7.09 (1H), 7.64 (1H), 8.01 (1H), 8.09 (1H), 8.12 (1H).

LC-MS (Method 15): Rₜ=0.96 min; MS (ESIpos) m/z=395 [M+H]⁺.

### Example III-008

### (2S)-1-({3-[4-(4-Methylpiperazin-1-yl)furo[3,2-c]pyridin-2-yl]imidazo[1,2-b]pyridazin-6-yl}oxy)propan-2-amine

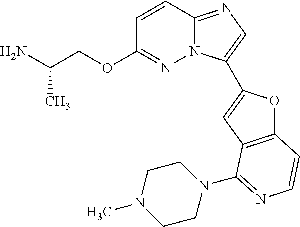 [SMILES: C[C@H](N)COc1ccc2ncc(-c3cc4c(N5CCN(C)CC5)nccc4o3)n2n1]

To a stirred suspension of (2S)-2-aminopropan-1-ol (61 mg) in anhydrous THF (10 mL) and anhydrous DMF (1.0 mL) was added sodium hydride (60% w/w in oil; 57 g) at 0° C. and the mixture was stirred at room temperature for 30 minutes. 6-chloro-3-[4-(4-methylpiperazin-1-yl)furo[3,2-c]pyridin-2-yl]imidazo[1,2-b]pyridazine (150 mg) was added and the mixture was stirred at room temperature for 4 hours. Water was added and the solvent was removed in vacuum. Silicagel chromatography gave a solid that dissolved in water and freeze dried to give 55 mg of the title compound.

¹H-NMR (400 MHz, DMSO-d₆, detected signals): δ [ppm]=1.11 (3H), 2.20 (3H), 3.61-3.74 (4H), 4.11-4.27 (2H), 7.03 (1H), 7.07 (1H), 7.66 (1H), 7.99 (1H), 8.10 (1H), 8.15 (1H).

LC-MS; MS (ESIpos) m/z=408 [M+H]⁺.

### Example III-009

### (2S)-1-({3-[4-(Piperidin-1-yl)furo[3,2-c]pyridin-2-yl]imidazo[1,2-b]pyridazin-6-yl}oxy)propan-2-amine

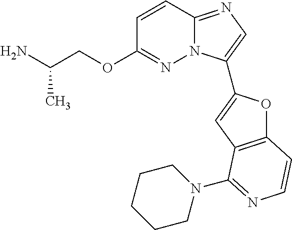 [SMILES: C[C@H](N)COc1ccc2ncc(-c3cc4c(N5CCCCC5)nccc4o3)n2n1]

To a stirred solution of (2S)-1-[(3-Bromoimidazo[1,2-b]pyridazin-6-yl)oxy]propan-2-amine (150 mg) in 1-propanol (18.5 mL) was added 2M potassium carbonate solution (0.83 mL), crude [4-(piperidin-1-yl)furo[3,2-c]pyridin-2-yl]boronic acid (204 mg), triphenylphosphine (14.5 mg) and PdCl₂(PPh₃)₂ (39.6 mg). The mixture was heated to reflux for 2 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with ethanol to give 60 mg of the title compound.

¹H-NMR (500 MHz, Pyr-d₅), δ [ppm]=1.29 (3H), 1.56-1.64 (2H), 1.67-1.76 (4H), 1.80-2.11 (2H), 3.53 (1H), 3.82-3.91 (4H), 4.24-4.36 (2H), 6.85 (1H), 7.11 (1H), 7.77 (1H), 8.07 (1H), 8.29 (1H), 8.45 (1H).

LC-MS; MS (ESIpos) m/z=393 [M+H]⁺.

### Example III-010

### (2S)-1-({3-[4-(Pyrrolidin-1-yl)furo[3,2-c]pyridin-2-yl]imidazo[1,2-b]pyridazin-6-yl}oxy)propan-2-amine

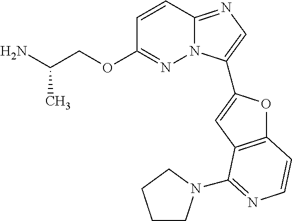 [SMILES: C[C@H](N)COc1ccc2ncc(-c3cc4c(N5CCCC5)nccc4o3)n2n1]

To a stirred solution of (2S)-1-[(3-Bromoimidazo[1,2-b]pyridazin-6-yl)oxy]propan-2-amine (150 mg) in 1-propanol (18.5 mL) was added 2M potassium carbonate solution (0.83 mL), crude [4-(pyrrolidin-1-yl)furo[3,2-c]pyridin-2-yl]boronic acid (25% w/w; 2.59 g), triphenylphosphine (14.5 mg) and PdCl₂(PPh₃)₂ (39.6 mg). The mixture was heated to reflux for 2 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with ethanol to give 70 mg of the title compound.

¹H-NMR (400 MHz, Pyr-d₅), δ [ppm]=1.21 (3H), 1.58-2.19 (6H), 3.44-3.54 (1H), 3.75-3.86 (4H), 4.17-4.30 (2H), 6.77 (1H), 6.97 (1H), 7.88 (1H), 8.00 (1H), 8.23 (1H), 8.38 (1H).

LC-MS; MS (ESIpos) m/z=379 [M+H]⁺.

### Example III-011

### (3R)-1-[2-(6-{[(2S)-2-Aminopropyl]oxy}imidazo[1,2-b]pyridazin-3-yl)furo[3,2-c]pyridin-4-yl]-N,N-dimethylpyrrolidin-3-amine

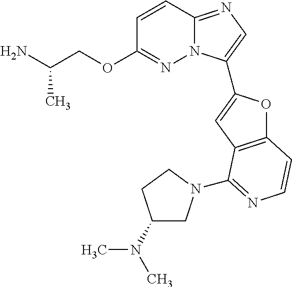 [SMILES: C[C@H](N)COc1ccc2ncc(-c3cc4c(N5CC[C@@H](N(C)C)C5)nccc4o3)n2n1]

To a stirred solution of (2S)-1-[(3-Bromoimidazo[1,2-b]pyridazin-6-yl)oxy]propan-2-amine (100 mg) in 1-propanol (10 mL) was added 2M potassium carbonate solution (0.55 mL), crude {4-[(3R)-3-(dimethylamino)pyrrolidin-1-yl]furo[3,2-c]pyridin-2-yl}boronic acid (65% w/w; 390 mg), triphenylphosphine (9.7 mg) and PdCl₂(PPh₃)₂ (25.9 mg). The mixture was heated to reflux for 1.5 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 35 mg of the title compound.

¹H-NMR (300 MHz, DMSO-d6), δ [ppm]=1.04-1.12 (3H), 1.48-1.72 (2H), 1.73-1.91 (1H), 2.19 (7H), 2.64-2.80 (1H), 3.14-3.25 (1H), 3.36 (1H), 3.58-3.74 (1H), 3.81-3.96 (2H), 4.04-4.17 (2H), 6.85 (1H), 6.95 (1H), 7.68 (1H), 7.89 (1H), 8.02 (1H), 8.08 (1H).

LC-MS; MS (ESIpos) m/z=422 [M+H]⁺.

### Example III-012

### 2-(6-{[(2S)-2-Aminopropyl]oxy}imidazo[1,2-b]pyridazin-3-yl)-N-methyl-N-[3-(pyrrolidin-1-yl)propyl]furo[3,2-c]pyridin-4-amine

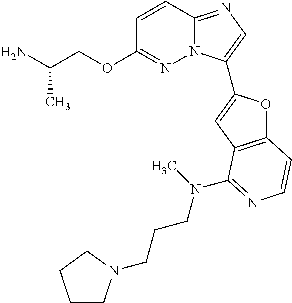 [SMILES: C[C@H](N)COc1ccc2ncc(-c3cc4c(N(C)CCCN5CCCC5)nccc4o3)n2n1]

To a stirred solution of (2S)-1-[(3-bromoimidazo[1,2-b]pyridazin-6-yl)oxy]propan-2-amine (100 mg) in 1-propanol (10 mL) was added 2M potassium carbonate solution (0.55 mL), crude (4-{methyl[3-(pyrrolidin-1-yl)propyl]amino}furo[3,2-c]pyridin-2-yl)boronic acid (52% w/w; 430 mg), triphenylphosphine (9.7 mg) and PdCl₂(PPh₃)₂ (25.9 mg). The mixture was heated to reflux for 1.5 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with ethanol to give 82 mg of the title compound.

¹H-NMR (400 MHz, DMSO-d₆), δ [ppm]=1.09 (3H), 1.57 (4H), 1.76 (2H), 2.30-2.43 (6H), 3.22-3.32 (6H), 3.75 (2H), 4.11-4.26 (2H), 6.89 (1H), 7.00 (1H), 7.79 (1H), 7.93 (1H), 8.06 (1H), 8.12 (1H).

LC-MS; MS (ESIpos) m/z=450 [M+H]⁺.

### Example III-013

### 2-(6-{[(2S)-2-Aminopropyl]oxy}imidazo[1,2-b]pyridazin-3-yl)-N-methyl-N-(1-methylpiperidin-4-yl)furo[3,2-c]pyridin-4-amine

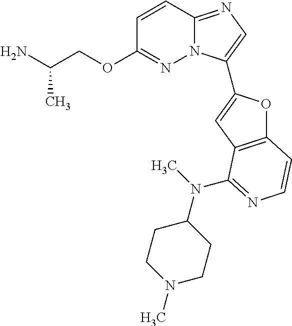 [SMILES: C[C@H](N)COc1ccc2ncc(-c3cc4c(N(C)C5CCN(C)CC5)nccc4o3)n2n1]

To a stirred solution of (2S)-1-[(3-Bromoimidazo[1,2-b]pyridazin-6-yl)oxy]propan-2-amine (100 mg) in 1-propanol (10 mL) was added 2M potassium carbonate solution (0.55 mL), crude {4-[methyl(1-methylpiperidin-4-yl)amino]furo[3,2-c]pyridin-2-yl}boronic acid (26% w/w; 820 mg), triphenylphosphine (9.7 mg) and PdCl₂(PPh₃)₂ (25.9 mg). The mixture was heated to reflux for 1.5 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 38 mg of the title compound.

¹H-NMR (400 MHz, DMSO-d₆, detected signals), δ [ppm]=1.16 (3H), 1.61 (2H), 1.75-1.90 (2H), 1.94-2.05 (2H), 2.17 (3H), 2.85 (2H), 3.16-3.20 (3H), 3.42 (1H), 4.20-4.38 (2H), 4.53-4.67 (1H), 6.94 (1H), 7.01 (1H), 7.75 (1H), 7.95 (1H), 8.10 (1H), 8.16 (1H).

LC-MS; MS (ESIpos) m/z=436 [M+H]⁺.

### Example III-014

### {(2R)-1-[2-(6-{[(2S)-2-Aminopropyl]oxy}imidazo[1,2-b]pyridazin-3-yl)furo-[3,2-c]pyridin-4-yl]pyrrolidin-2-yl}methanol

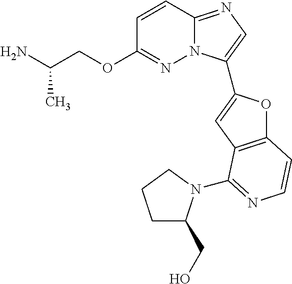 [SMILES: C[C@H](N)COc1ccc2ncc(-c3cc4c(N5CCC[C@@H]5CO)nccc4o3)n2n1]

To a stirred solution of (2S)-1-[(3-{4-[(2R)-2-({[tert-butyl(dimethyl)silyl]oxy}methyl)pyrrolidin-1-yl]furo[3,2-c]pyridin-2-yl}imidazo[1,2-b]pyridazin-6-yl)oxy]propan-2-amine (150 mg) in THF (15 mL) was added a solution of TBAF in THF (0.57 mL; c=1.0 mol/L). The mixture was stirred at room temperature for 4 h. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 65 mg of the title compound.

¹H-NMR (400 MHz, DMSO-d₆), δ [ppm]=1.08 (3H), 1.64 (2H), 1.84-2.10 (4H), 3.19-3.25 (1H), 3.32-3.41 (1H), 3.59-3.72 (2H), 3.86 (1H), 4.10-4.25 (2H), 4.37 (1H), 5.06 (1H), 6.87 (1H), 6.96 (1H), 7.77 (1H), 7.91 (1H), 8.04 (1H), 8.09 (1H).

LC-MS; MS (ESIpos) m/z=409 [M+H]⁺.

### Example III-015

### tert-Butyl 4-[2-(6-{[(2S)-2-aminopropyl]oxy}imidazo[1,2-b]pyridazin-3-yl)furo[3,2-c]pyridin-4-yl]piperazine-1-carboxylate

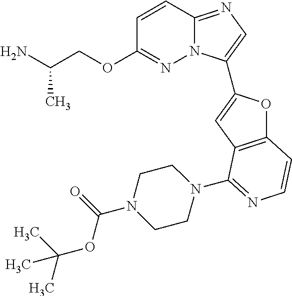 [SMILES: C[C@H](N)COc1ccc2ncc(-c3cc4c(N5CCN(C(=O)OC(C)(C)C)CC5)nccc4o3)n2n1]

To a stirred solution of (2S)-1-[(3-Bromoimidazo[1,2-b]pyridazin-6-yl)oxy]propan-2-amine (120 mg) in 1-propanol (15 mL) was added 2M potassium carbonate solution (0.66 mL), crude {4-[4-(tert-butoxycarbonyl)piperazin-1-yl]furo[3,2-c]pyridin-2-yl}boronic acid (90% w/w; 341 mg), triphenylphosphine (11.6 mg) and PdCl₂(PPh₃)₂ (31.7 mg). The mixture was heated to reflux for 2 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with a mixture of 2-propanol and ether to give 120 mg of the title compound.

¹H-NMR (400 MHz, Pyr-d5), δ [ppm]=1.33 (3H), 1.54 (9H), 1.82-2.01 (2H), 3.45-3.59 (1H), 3.79 (4H), 3.91 (4H), 4.31 (2H), 6.83 (1H), 7.15 (1H), 7.76 (1H), 8.05 (1H), 8.26 (1H), 8.43 (1H).

LC-MS; MS (ESIpos) m/z=494 [M+H]⁺.

### Example III-016

### 2-(6-{[(2S)-2-Aminopropyl]oxy}imidazo[1,2-b]pyridazin-3-yl)-N-(2-methoxyethyl)-N-methylfuro[3,2-c]pyridin-4-amine

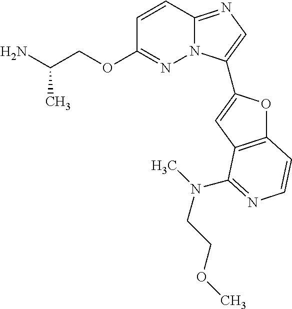 [SMILES: COCCN(C)c1nccc2oc(-c3cnc4ccc(OC[C@H](C)N)nn34)cc12]

To a stirred solution of (2S)-1-[(3-Bromoimidazo[1,2-b]pyridazin-6-yl)oxy]propan-2-amine (80 mg) in 1-propanol (10 mL) was added 2M potassium carbonate solution (0.44 mL), crude {4-[(2-methoxyethyl)(methyl)amino]furo[3,2-c]pyridin-2-yl}boronic acid (80% w/w; 185 mg), triphenylphosphine (7.7 mg) and PdCl₂(PPh₃)₂ (21.1 mg). The mixture was heated to reflux for 2 h. The reaction mixture was filtered through an aminophase silicagel column and the solvent was removed in vacuum. Silicagel chromatography gave a solid that was triturated with 2-propanol to give 70 mg of the title compound.

¹H-NMR (400 MHz, Pyr-d₅, detected signals), δ [ppm]=1.30 (3H), 3.31 (3H), 3.49-3.61 (4H), 3.70-3.79 (2H), 4.05 (2H), 4.34 (2H), 6.80 (1H), 7.02 (1H), 7.89 (1H), 8.03 (1H), 8.24 (1H), 8.40 (1H).

LC-MS; MS (ESIpos) m/z=397 [M+H]⁺.

### Example III-017

### (2S)-1-({3-[4-(Piperazin-1-yl)furo[3,2-c]pyridin-2-yl]imidazo[1,2-b]pyridazin-6-yl}oxy)propan-2-amine

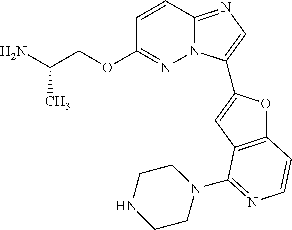 [SMILES: C[C@H](N)COc1ccc2ncc(-c3cc4c(N5CCNCC5)nccc4o3)n2n1]

To a stirred suspension of tert-butyl 4-[2-(6-{[(2S)-2-aminopropyl]oxy}imidazo[1,2-b]pyridazin-3-yl)furo[3,2-c]pyridin-4-yl]piperazine-1-carboxylate (180 mg) in dichloromethane (12 mL) was added TFA (0.69 mL). The mixture was stirred at room temperature for 16 h. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was triturated with ethyl acetate to give 110 mg of the title compound.

¹H-NMR (400 MHz, Pyr-d₅, detected signals), δ [ppm]=1.20-1.27 (3H), 3.08-3.16 (4H), 3.43-3.56 (1H), 3.90-3.97 (4H), 4.26-4.32 (2H), 6.83 (1H), 7.13 (1H), 7.79 (1H), 8.05 (1H), 8.29 (1H), 8.43 (1H).

LC-MS; MS (ESIpos) m/z=394 [M+H]⁺.

### Example III-018

### 2-(6-{[(2S)-2-Aminopropyl]oxy}imidazo[1,2-b]pyridazin-3-yl)-N-ethyl-N-(2-methoxyethyl)furo[3,2-c]pyridin-4-amine

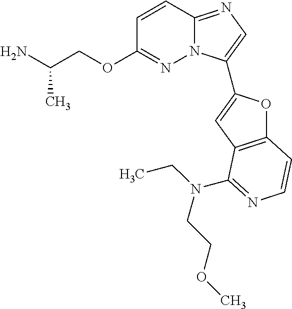 [SMILES: CCN(CCOC)c1nccc2oc(-c3cnc4ccc(OC[C@H](C)N)nn34)cc12]

To a stirred solution of (2S)-1-[(3-Bromoimidazo[1,2-b]pyridazin-6-yl)oxy]propan-2-amine (2.2 g) in 1-propanol (200 mL) was added 2M potassium carbonate solution (12.2 mL), crude {4-[Ethyl(2-methoxyethyl)amino]furo[3,2-c]pyridin-2-yl}boronic acid (80% w/w; 4.29 g), triphenylphosphine (213 mg) and PdCl₂(PPh₃)₂ (581 mg). The mixture was heated to reflux for 3 h. The reaction mixture was filtered through an aminophase silicagel column and the solvent was removed in vacuum. Silicagel chromatography gave a solid that was triturated with a mixture of ethyl acetate and hexane to give 2.5 g of the title compound.

¹H-NMR (300 MHz, DMSO-d₆), δ [ppm]=1.09 (3H), 1.24 (3H), 1.94 (2H), 3.16-3.29 (4H), 3.53-3.62 (2H), 3.67-3.86 (4H), 4.15 (2H), 6.88 (1H), 6.98 (1H), 7.58 (1H), 7.91 (1H), 8.05 (1H), 8.11 (1H).

LC-MS; MS (ESIpos) m/z=411 [M+H]⁺.

### Example III-019

### (2S)-1-[(3-{4-[(2R,6S)-2,6-Dimethylmorpholin-4-yl]furo[3,2-c]pyridin-2-yl}imidazo[1,2-b]pyridazin-6-yl)oxy]propan-2-amine

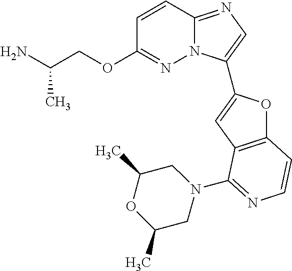 [SMILES: C[C@H](N)COc1ccc2ncc(-c3cc4c(N5C[C@@H](C)O[C@@H](C)C5)nccc4o3)n2n1]

To a stirred solution of (2S)-1-[(3-Bromoimidazo[1,2-b]pyridazin-6-yl)oxy]propan-2-amine (100 mg) in 1-propanol (10 mL) was added 2M potassium carbonate solution (0.55 mL), crude {4-[(2R,6S)-2,6-dimethylmorpholin-4-yl]furo[3,2-c]pyridin-2-yl}boronic acid (90% w/w; 272 mg), triphenylphosphine (9.7 mg) and PdCl₂(PPh₃)₂ (25.9 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with a mixture of dichloromethane and hexane to give 104 mg of the title compound.

¹H-NMR (300 MHz, CHLOROFORM-d, detected signals), δ [ppm]=1.21-1.35 (9H), 2.84 (2H), 3.48 (1H), 3.77-3.93 (2H), 4.16-4.28 (3H), 4.39 (1H), 6.84 (1H), 7.01 (1H), 7.54 (1H), 7.92 (1H), 8.08 (1H), 8.15 (1H).

LC-MS; MS (ESIpos) m/z=423 [M+H]⁺.

### Example III-020

### 3-{[2-(6-{[(2S)-2-Aminopropyl]oxy}imidazo[1,2-b]pyridazin-3-yl)furo[3,2-c]pyridin-4-yl](methyl)amino}propan-1-ol

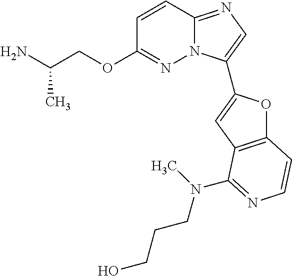 [SMILES: C[C@H](N)COc1ccc2ncc(-c3cc4c(N(C)CCCO)nccc4o3)n2n1]

To a stirred solution of 2-(6-{[(2S)-2-aminopropyl]oxy}imidazo[1,2-b]pyridazin-3-yl)-N-(3-{[tert-butyl(dimethyl)silyl]oxy}propyl)-N-methylfuro[3,2-c]pyridin-4-amine (160 mg) in THF (16 mL) was added a solution of TBAF in THF (0.63 mL; c=1.0 mol/L). The mixture was stirred at room temperature for 4 h. A half-saturated solution of sodium bicarbonate was added and the mixture was extracted with a mixture of ethyl acetate and methanol. The organic phase was dried (sodium sulfate) and the solvent was removed in vacuum. Silicagel chromatography gave a solid that was triturated with a mixture of dichloromethane and hexane to give 66 mg of the title compound.

¹H-NMR (300 MHz, DMSO-d₆), δ [ppm]=1.08 (3H), 1.44-1.88 (4H), 3.18-3.28 (4H), 3.46 (2H), 3.74 (2H), 4.17 (2H), 4.79 (1H), 6.89 (1H), 6.99 (1H), 7.77 (1H), 7.92 (1H), 8.06 (1H), 8.12 (1H).

LC-MS; MS (ESIpos) m/z=397 [M+H]⁺.

### Example III-021

### 1-[2-(6-{[(2S)-2-Aminopropyl]oxy}imidazo[1,2-b]pyridazin-3-yl)furo[3,2-c]pyridin-4-yl]-N,N-dimethylpiperidin-4-amine

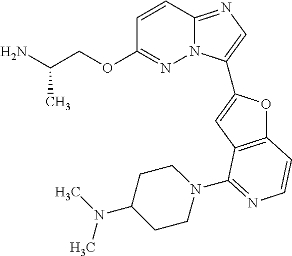 [SMILES: C[C@H](N)COc1ccc2ncc(-c3cc4c(N5CCC(N(C)C)CC5)nccc4o3)n2n1]

To a stirred solution of (2S)-1-[(3-Bromoimidazo[1,2-b]pyridazin-6-yl)oxy]propan-2-amine (80 mg) in 1-propanol (10 mL) was added 2M potassium carbonate solution (0.44 mL), crude {4-[4-(dimethylamino)piperidin-1-yl]furo[3,2-c]pyridin-2-yl}boronic acid (85% w/w; 220 mg), triphenylphosphine (7.7 mg) and PdCl₂(PPh₃)₂ (21.1 mg). The mixture was heated to reflux for 4 h. The reaction mixture was filtered through an aminophase silicagel column and the solvent was removed in vacuum. Aminophase silicagel chromatography gave a solid that was triturated with a mixture of ethyl acetate and hexane to give 50 mg of the title compound.

¹H-NMR (400 MHz, CHLOROFORM-d, detected signals), δ [ppm]=1.27 (3H), 1.68 (2H), 1.99 (2H), 2.33 (6H), 2.38-2.49 (1H), 3.07 (2H), 3.44-3.55 (1H), 4.21 (1H), 4.39 (1H), 4.49 (2H), 6.83 (1H), 6.97 (1H), 7.59 (1H), 7.92 (1H), 8.07 (1H), 8.14 (1H).

### Example III-022

### {1-[2-(6-{[(2S)-2-Aminopropyl]oxy}imidazo[1,2-b]pyridazin-3-yl)furo[3,2-c]pyridin-4-yl]piperidin-4-yl}methanol

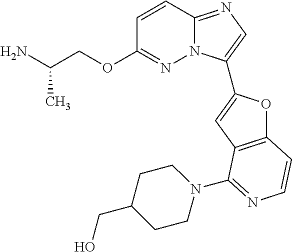 [SMILES: C[C@H](N)COc1ccc2ncc(-c3cc4c(N5CCC(CO)CC5)nccc4o3)n2n1]

To a stirred solution of (2S)-1-[(3-{4-[4-({[tert-butyl(dimethyl)silyl]oxy}methyl) piperidin-1-yl]furo[3,2-c]pyridin-2-yl}imidazo[1,2-b]pyridazin-6-yl)oxy]propan-2-amine (140 mg) in THF (12.5 mL) was added a solution of TBAF in THF (0.65 mL; c=1.0 mol/L). The mixture was stirred at room temperature for 48 h. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with ethyl acetate to give 70 mg of the title compound.

¹H-NMR (300 MHz, DMSO-d₆), δ [ppm]=1.09 (3H), 1.15-1.33 (2H), 1.57-1.86 (5H), 2.98 (2H), 3.16-3.36 (3H), 4.05-4.22 (2H), 4.30-4.57 (3H), 6.96-7.02 (2H), 7.59 (1H), 7.95 (1H), 8.07 (1H), 8.12 (1H).

### Example III-023

### (2S)-1-({3-[4-(4-Phenylpiperazin-1-yl)furo[3,2-c]pyridin-2-yl]imidazo[1,2-b]pyridazin-6-yl}oxy)propan-2-amine

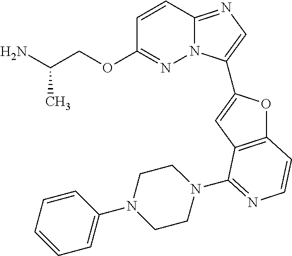 [SMILES: C[C@H](N)COc1ccc2ncc(-c3cc4c(N5CCN(c6ccccc6)CC5)nccc4o3)n2n1]

To 135 mg (0.5 mmol) (2S)-1-[(3-bromoimidazo[1,2-b]pyridazin-6-yl)oxy]propan-2-amine in 6 mL propan-1-ol were added 242 mg (0.75 mmol) [4-(4-phenylpiperazin-1-yl)furo[3,2-c]pyridin-2-yl]boronic acid, 13 mg (50 mol) triphenylphosphin, 29 mg (50 mol) Pd(dba)₂ and 0.75 mL of a 2 M aqueous solution of sodium carbonate. The mixture was stirred at 110° C. for 2 h.

Water was added. The mixture was extracted with dichloromethane. The organic layer was separated, dried over sodium sulfate, and evaporated. The crude product was purified by HPLC to give 22 mg of the title compound.

¹H-NMR (400 MHz, DMSO-d₆), δ [ppm]=1.16 (3H), 3.33-3.38 (4H), 3.81-3.88 (4H), 4.25-4.30 (2H), 6.78 (1H), 6.96 (2H), 7.04 (1H), 7.12 (1H), 7.22 (2H), 7.74 (1H), 8.04 (1H), 8.13 (1H), 8.16 (1H).

LC-MS (Method 13): Rₜ=0.72 min; MS (ESIpos) m/z=235 [M+H]⁺⁺.

### Example III-024

### (2S)-1-[(3-{4-[(2R)-2-(Methoxymethyl)pyrrolidin-1-yl]furo[3,2-c]pyridin-2-yl}imidazo[1,2-b]pyridazin-6-yl)oxy]propan-2-amine

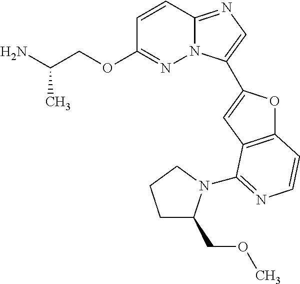 [SMILES: COC[C@H]1CCCN1c1nccc2oc(-c3cnc4ccc(OC[C@H](C)N)nn34)cc12]

To a stirred solution of (2S)-1-[(3-Bromoimidazo[1,2-b]pyridazin-6-yl)oxy]propan-2-amine (80 mg) in 1-propanol (10 mL) was added 2M potassium carbonate solution (0.44 mL), crude {4-[(2R)-2-(methoxymethyl)pyrrolidin-1-yl]furo[3,2-c]pyridin-2-yl}boronic acid (80% w/w; 152 mg), triphenylphosphine (7.7 mg) and PdCl₂(PPh₃)₂ (21.1 mg). The mixture was heated to reflux for 2 h. The reaction mixture was filtered and the solvent was removed in vacuum. Silicagel chromatography followed by aminophase silicagel chromatography gave a solid that was triturated with ethyl acetate to give 80 mg of the title compound.

¹H-NMR (400 MHz, Pyr-d₅), δ [ppm]=1.24 (3H), 1.68-2.14 (6H), 3.27 (3H), 3.39 (1H), 3.44-3.55 (1H), 3.73-3.84 (2H), 4.01-4.10 (1H), 4.26-4.33 (2H), 4.74-4.80 (1H), 6.77 (1H), 6.99 (1H), 7.90 (1H), 7.99 (1H), 8.22 (1H), 8.38 (1H).

### Example III-025

### (2S)-1-[(3-{4-[(3R)-3-Methylmorpholin-4-yl]furo[3,2-c]pyridin-2-yl}imidazo-[1,2-b]pyridazin-6-yl)oxy]propan-2-amine

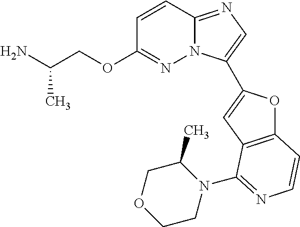 [SMILES: C[C@H](N)COc1ccc2ncc(-c3cc4c(N5CCOC[C@H]5C)nccc4o3)n2n1]

To 100 mg (0.37 mmol) (2S)-1-[(3-bromoimidazo[1,2-b]pyridazin-6-yl)oxy]propan-2-amine in 6 mL propan-1-ol were added 322 mg (0.74 mmol) {4-[(3R)-3-methylmorpholin-4-yl]furo[3,2-c]pyridin-2-yl}boronic acid, 17 mg (15 μmol) tetrakis(triphenylphosphin)palladium(0) and 0.55 mL of a 2 M aqueous solution of sodium carbonate. The mixture was stirred at 110° C. for 17 h.

The reaction mixture was poured into water and extracted with dichloromethane. The organic layer was dried over sodium sulfate, and concentrated. The crude material was digested in methanol to give 58 mg of the title compound.

¹H-NMR (400 MHz, DMSO-d₆), δ [ppm]=1.10 (3H), 1.26 (3H), 3.18-3.27 (1H), 3.34-3.41 (1H), 3.56 (1H), 3.72 (2H), 3.90-3.99 (1H), 4.08-4.17 (2H), 4.19-4.27 (1H), 4.52 (1H), 6.99-7.07 (2H), 7.69 (1H), 8.00 (1H), 8.10 (1H), 8.12-8.18 (1H).

LC-MS (Method 13): Rₜ=0.58 min; MS (ESIpos) m/z=205 [M+H]⁺⁺.

### Example III-026

### 2-(6-{[(2S)-2-Aminopropyl]oxy}imidazo[1,2-b]pyridazin-3-yl)-N-(2-tert-butoxyethyl)-N-ethylfuro[3,2-c]pyridin-4-amine

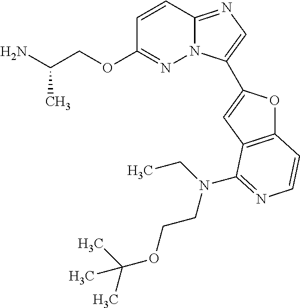 [SMILES: CCN(CCOC(C)(C)C)c1nccc2oc(-c3cnc4ccc(OC[C@H](C)N)nn34)cc12]

To a stirred solution of (2S)-1-[(3-Bromoimidazo[1,2-b]pyridazin-6-yl)oxy]propan-2-amine (80 mg) in 1-propanol (10 mL) was added 2M potassium carbonate solution (0.44 mL), crude {4-[(2-tert-butoxyethyl)(ethyl)amino]furo[3,2-c]pyridin-2-yl}boronic acid (50% w/w; 361 mg), triphenylphosphine (7.7 mg) and PdCl₂(PPh₃)₂ (21.1 mg). The mixture was heated to reflux for 2 h. The reaction mixture was filtered and the solvent was removed in vacuum. Silicagel chromatography followed by aminophase silicagel chromatography gave a solid that was triturated with a mixture of ethyl acetate and hexane to give 80 mg of the title compound.

¹H-NMR (300 MHz, DMSO-d₆), δ [ppm]=1.00-1.13 (12H), 1.26 (3H), 1.64 (2H), 3.18-3.27 (1H), 3.49-3.60 (2H), 3.68-3.82 (4H), 4.09-4.26 (2H), 6.88 (1H), 7.00 (1H), 7.65 (1H), 7.92 (1H), 8.06 (1H), 8.13 (1H).

### Example III-027

### (2S)-1-[(3-{4-[(2S)-2-(Methoxymethyl)pyrrolidin-1-yl]furo[3,2-c]pyridin-2-yl}imidazo[1,2-b]pyridazin-6-yl)oxy]propan-2-amine

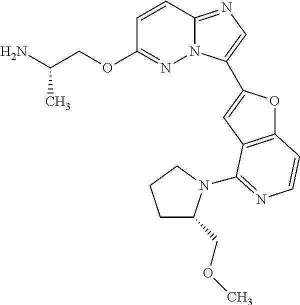 [SMILES: COC[C@@H]1CCCN1c1nccc2oc(-c3cnc4ccc(OC[C@H](C)N)nn34)cc12]

To a stirred solution of (2S)-1-[(3-Bromoimidazo[1,2-b]pyridazin-6-yl)oxy]propan-2-amine (100 mg) in 1-propanol (5 mL) was added 2M potassium carbonate solution (0.55 mL), crude {4-[(2S)-2-(methoxymethyl)pyrrolidin-1-yl]furo[3,2-c]pyridin-2-yl}boronic acid (82% w/w; 248 mg), triphenylphosphine (9.7 mg) and PdCl₂(PPh₃)₂ (25.9 mg). The mixture was heated to reflux for 2 h. The warm reaction mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave 115 mg of the title compound.

¹H-NMR (400 MHz, Pyr-d₅), δ [ppm]=1.24 (3H), 1.75-1.96 (4H), 1.97-2.12 (2H), 3.26 (3H), 3.38 (1H), 3.46-3.56 (1H), 3.72-3.84 (2H), 4.01-4.11 (1H), 4.23-4.36 (2H), 4.78 (1H), 6.78 (1H), 6.99 (1H), 7.91 (1H), 7.99 (1H), 8.23 (1H), 8.38 (1H).

### Example III-028

### (2S)-1-[(3-{4-[(3S)-3-Methylmorpholin-4-yl]furo[3,2-c]pyridin-2-yl}imidazo-[1,2-b]pyridazin-6-yl)oxy]propan-2-amine

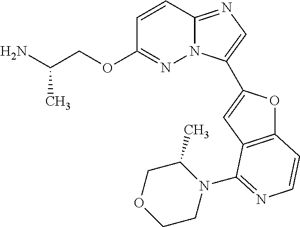 [SMILES: C[C@H](N)COc1ccc2ncc(-c3cc4c(N5CCOC[C@@H]5C)nccc4o3)n2n1]

To 100 mg (0.37 mmol) (2S)-1-[(3-bromoimidazo[1,2-b]pyridazin-6-yl)oxy]propan-2-amine in 6 mL propan-1-ol were added 322 mg (0.74 mmol) {4-[(35)-3-methylmorpholin-4-yl]furo[3,2-c]pyridin-2-yl}boronic acid, 17 mg (15 μmol) tetrakis(triphenylphosphin)palladium(0) and 0.55 mL of a 2 M aqueous solution of sodium carbonate. The mixture was stirred at 110° C. for 17 h.

Half-saturated brine was added. The mixture was extracted with dichloromethane. The organic layer was separated, dried over sodium sulfate, and evaporated. The crude product was purified by HPLC to give 58 mg of the title compound.

¹H-NMR (400 MHz, DMSO-d₆), δ [ppm]=1.09-1.16 (3H), 1.28 (3H), 3.35-3.45 (1H), 3.59 (1H), 3.75 (2H), 3.93-4.02 (1H), 4.10-4.29 (3H), 4.55 (1H), 7.01-7.12 (2H), 7.72 (1H), 8.03 (1H), 8.10-8.20 (2H).

### Example III-029

### (2S)-1-[(3-{4-[(2S)-2-Methylmorpholin-4-yl]furo[3,2-c]pyridin-2-yl}imidazo-[1,2-b]pyridazin-6-yl)oxy]propan-2-amine

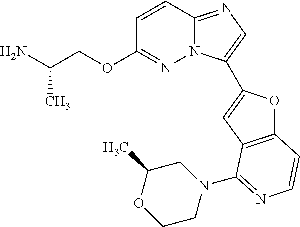 [SMILES: C[C@H](N)COc1ccc2ncc(-c3cc4c(N5CCO[C@@H](C)C5)nccc4o3)n2n1]

To 100 mg (0.37 mmol) (2S)-1-[(3-bromoimidazo[1,2-b]pyridazin-6-yl)oxy]propan-2-amine in 6 mL propan-1-ol were added 312 mg (0.74 mmol) {4-[(2S)-2-methylmorpholin-4-yl]furo[3,2-c]pyridin-2-yl}boronic acid, 17 mg (15 μmol) tetrakis(triphenylphosphin)palladium(0) and 0.55 mL of a 2 M aqueous solution of sodium carbonate. The mixture was stirred at 110° C. for 18 h.

Water was added. The mixture was extracted with dichloromethane. The organic layer was separated, dried over sodium sulfate, and evaporated. The crude product was purified by flash chromatography to give 107 mg of the title compound.

¹H-NMR (400 MHz, DMSO-d₆), δ [ppm]1.14-1.23 (6H), 2.76 (1H), 3.15 (1H), 3.62-3.75 (2H), 3.95 (1H), 4.11-4.36 (4H), 7.04 (1H), 7.13 (1H), 7.67 (1H), 8.03 (1H), 8.15 (1H), 8.18 (1H).

LC-MS (Method 14): Rₜ=0.57 min; MS (ESIpos) m/z=409 [M+H]⁺.

### Example III-030

### (2R)-2-({3-[4-(Morpholin-4-yl)furo[3,2-c]pyridin-2-yl]imidazo[1,2-b]pyridazin-6-yl}oxy)propan-1-amine

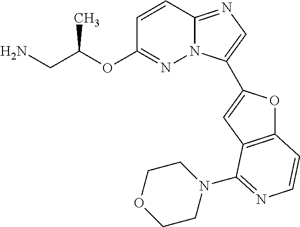 [SMILES: C[C@H](CN)Oc1ccc2ncc(-c3cc4c(N5CCOCC5)nccc4o3)n2n1]

To a stirred solution of (2R)-2-[(3-bromoimidazo[1,2-b]pyridazin-6-yl)oxy]propan-1-amine (100 mg) in 1-propanol (10 mL) was added 2M potassium carbonate solution (0.55 mL), crude [4-(morpholin-4-yl)furo[3,2-c]pyridin-2-yl]boronic acid (80% w/w; 229 mg), triphenylphosphine (9.7 mg) and PdCl₂(PPh₃)₂ (25.9 mg). The mixture was heated to reflux for 1 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave 107 mg of the title compound.

¹H-NMR (300 MHz, DMSO-d₆), δ [ppm]=1.41 (3H), 1.59 (2H), 2.76-2.92 (2H), 3.57-3.70 (4H), 3.71-3.85 (4H), 5.05 (1H), 6.97 (1H), 7.11 (1H), 7.66 (1H), 8.01 (1H), 8.09-8.16 (2H).

### Example III-031

### (2R)-2-[(3-{4-[(2R)-2-(Methoxymethyl)pyrrolidin-1-yl]furo[3,2-c]pyridin-2-yl}imidazo[1,2-b]pyridazin-6-yl)oxy]propan-1-amine

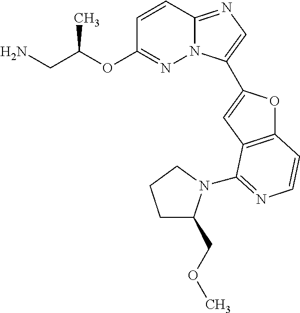 [SMILES: COC[C@H]1CCCN1c1nccc2oc(-c3cnc4ccc(O[C@H](C)CN)nn34)cc12]

To a stirred solution of (2R)-2-[(3-bromoimidazo[1,2-b]pyridazin-6-yl)oxy]propan-1-amine (100 mg) in 1-propanol (10 mL) was added 2M potassium carbonate solution (0.55 mL), crude {4-[(2R)-2-(methoxymethyl)pyrrolidin-1-yl]furo[3,2-c]pyridin-2-yl}boronic acid (80% w/w; 255 mg), triphenylphosphine (9.7 mg) and PdCl₂(PPh₃)₂ (25.9 mg). The mixture was heated to reflux for 1 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave 94 mg of the title compound.

¹H-NMR (300 MHz, DMSO-d₆), δ [ppm]=1.39 (3H), 1.53 (2H), 1.81-2.20 (5H), 2.84 (2H), 3.23 (3H), 3.52-3.73 (2H), 3.88-4.00 (1H), 4.43-4.55 (1H), 5.07 (1H), 6.89 (1H), 6.94 (1H), 7.74 (1H), 7.93 (1H), 8.05 (1H), 8.10 (1H).

### Example III-032

### 2-(6-{[(2R)-1-Aminopropan-2-yl]oxy}imidazo[1,2-b]pyridazin-3-yl)-N-ethyl-N-(2-methoxyethyl)furo[3,2-c]pyridin-4-amine

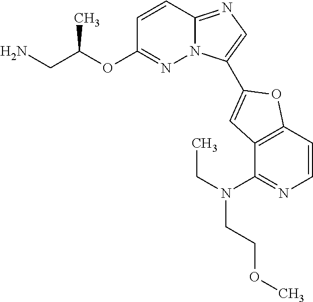 [SMILES: CCN(CCOC)c1nccc2oc(-c3cnc4ccc(O[C@H](C)CN)nn34)cc12]

To a stirred solution of (2R)-2-[(3-bromoimidazo[1,2-b]pyridazin-6-yl)oxy]propan-1-amine (400 mg) in 1-propanol (20 mL) was added 2M potassium carbonate solution (2.21 mL), crude {4-[Ethyl(2-methoxyethyl)amino]furo[3,2-c]pyridin-2-yl}boronic acid (82% w/w; 950 mg), triphenylphosphine (38.7 mg) and PdCl₂(PPh₃)₂ (104 mg). The mixture was heated to reflux for 1 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography followed by silicagel chromatography gave 360 mg of the title compound.

¹H-NMR (400 MHz, DMSO-d₆), δ [ppm]=1.26 (3H), 1.42 (3H), 1.76 (2H), 2.83-2.93 (2H), 3.28 (3H), 3.58-3.64 (2H), 3.74-3.82 (2H), 3.82-3.87 (2H), 5.07-5.19 (1H), 6.92 (1H), 6.97 (1H), 7.61 (1H), 7.95 (1H), 8.09 (1H), 8.14 (1H).

### Example III-033

### 2-(6-{[(2R)-1-Aminopropan-2-yl]oxy}imidazo[1,2-b]pyridazin-3-yl)-N-(2-methoxyethyl)-N-propylfuro[3,2-c]pyridin-4-amine

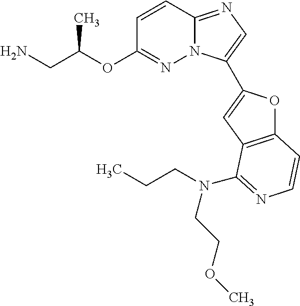 [SMILES: CCCN(CCOC)c1nccc2oc(-c3cnc4ccc(O[C@H](C)CN)nn34)cc12]

To a stirred solution of (2R)-2-[(3-bromoimidazo[1,2-b]pyridazin-6-yl)oxy]propan-1-amine (65 mg) in 1-propanol (5.0 mL) was added 2M potassium carbonate solution (0.36 mL), crude {4-[(2-methoxyethyl)(propyl)amino]furo[3,2-c]pyridin-2-yl}boronic acid (70% w/w; 192 mg), triphenylphosphine (6.3 mg) and PdCl₂(PPh₃)₂ (17 mg). The mixture was heated to reflux for 2 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave 80 mg of the title compound.

¹H-NMR (300 MHz, DMSO-d₆), δ [ppm]=0.90 (3H), 1.39 (3H), 1.50-1.77 (4H), 2.84 (2H), 3.25 (3H), 3.54-3.72 (4H), 3.77-3.91 (2H), 5.02-5.19 (1H), 6.91 (1H), 6.96 (1H), 7.55 (1H), 7.93 (1H), 8.07 (1H), 8.12 (1H).

### Example III-034

### 2-(6-{[(2R)-1-Aminopropan-2-yl]oxy}imidazo[1,2-b]pyridazin-3-yl)-N-(2-methoxyethyl)-N-methylfuro[3,2-c]pyridin-4-amine

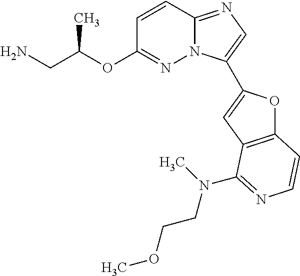 [SMILES: COCCN(C)c1nccc2oc(-c3cnc4ccc(O[C@H](C)CN)nn34)cc12]

To a stirred solution of (2R)-2-[(3-bromoimidazo[1,2-b]pyridazin-6-yl)oxy]propan-1-amine (100 mg) in 1-propanol (10 mL) was added 2M potassium carbonate solution (0.55 mL), {4-[(2-methoxyethyl)(methyl)amino]furo[3,2-c]pyridin-2-yl}boronic acid (90% w/w; 205 mg), triphenylphosphine (9.7 mg) and PdCl₂(PPh₃)₂ (25.9 mg). The mixture was heated to reflux for 1 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 102 mg of the title compound.

¹H-NMR (300 MHz, DMSO-d₆), δ [ppm]=1.39 (3H), 1.53 (2H), 2.84 (2H), 3.25 (3H), 3.33 (3H), 3.53-3.64 (2H), 3.80-3.91 (2H), 5.05 (1H), 6.88-6.98 (2H), 7.72 (1H), 7.92 (1H), 8.06 (1H), 8.08-8.14 (1H).

### Example III-035

### N-Ethyl-N-(2-methoxyethyl)-2-{6-[(3S)-morpholin-3-ylmethoxy]imidazo[1,2-b]pyridazin-3-yl}furo[3,2-c]pyridin-4-amine

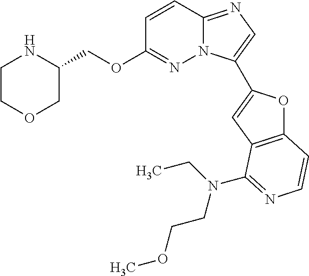 [SMILES: CCN(CCOC)c1nccc2oc(-c3cnc4ccc(OC[C@@H]5COCCN5)nn34)cc12]

To a stirred solution of 3-bromo-6-[(3S)-morpholin-3-ylmethoxy]imidazo[1,2-b]pyridazine (80 mg) in 1-propanol (5.0 mL) was added 2M potassium carbonate solution (0.38 mL), crude {4-[Ethyl(2-methoxyethyl)amino]furo[3,2-c]pyridin-2-yl}boronic acid (80% w/w; 222 mg), triphenylphosphine (6.7 mg) and PdCl₂(PPh₃)₂ (18.3 mg). The mixture was heated to reflux for 2 h. The reaction mixture was filtered through an aminophase silicagel column and the solvent was removed in vacuum. Silicagel chromatography gave 85 mg of the title compound.

¹H-NMR (400 MHz, DMSO-d₆), δ [ppm]=1.24 (3H), 2.60 (1H), 2.69-2.85 (2H), 3.09-3.18 (1H), 3.26 (3H), 3.39 (1H), 3.56-3.63 (2H), 3.63-3.69 (1H), 3.74 (2H), 3.79-3.87 (3H), 4.29 (2H), 6.89 (1H), 6.99 (1H), 7.60 (1H), 7.92 (1H), 8.07 (1H), 8.13 (1H).

### Example III-036

### 3-[4-(Morpholin-4-yl)furo[3,2-c]pyridin-2-yl]-6-[(3S)-morpholin-3-ylmethoxy]imidazo[1,2-b]pyridazine

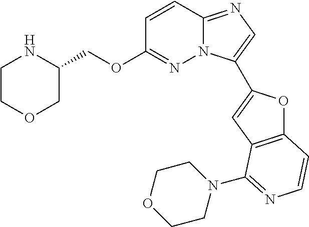 [SMILES: c1cc2oc(-c3cnc4ccc(OC[C@@H]5COCCN5)nn34)cc2c(N2CCOCC2)n1]

To a stirred solution of 3-bromo-6-[(3S)-morpholin-3-ylmethoxy]imidazo[1,2-b]pyridazine (80 mg) in 1-propanol (5.0 mL) was added 2M potassium carbonate solution (0.38 mL), crude [4-(morpholin-4-yl)furo[3,2-c]pyridin-2-yl]boronic acid (80% w/w; 158 mg), triphenylphosphine (6.7 mg) and PdCl₂(PPh₃)₂ (18.3 mg). The mixture was heated to reflux for 2 h. The reaction mixture was filtered through an aminophase silicagel column and the solvent was removed in vacuum. Silicagel chromatography followed by aminophase silicagel chromatography gave a solid that was triturated with warm ethanol to give 55 mg of the title compound.

¹H-NMR (300 MHz, DMSO-d₆), δ [ppm]=2.61 (1H), 2.72-2.84 (2H), 3.14 (1H), 3.25 (1H), 3.34-3.46 (1H), 3.59-3.70 (5H), 3.72-3.89 (5H), 4.30 (2H), 6.99 (1H), 7.09 (1H), 7.63 (1H), 8.00 (1H), 8.09-8.17 (1H).

### Example III-037

### 3-{4-[(2R)-2-(Methoxymethyl)pyrrolidin-1-yl]furo[3,2-c]pyridin-2-yl}-6-[(3S)-morpholin-3-ylmethoxy]imidazo[1,2-b]pyridazine

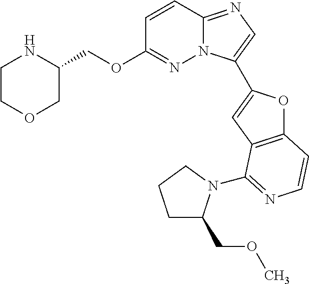 [SMILES: COC[C@H]1CCCN1c1nccc2oc(-c3cnc4ccc(OC[C@@H]5COCCN5)nn34)cc12]

To a stirred solution of 3-bromo-6-[(3S)-morpholin-3-ylmethoxy]imidazo[1,2-b]pyridazine (80 mg) in 1-propanol (8.5 mL) was added 2M potassium carbonate solution (0.38 mL), crude [4-(morpholin-4-yl)furo[3,2-c]pyridin-2-yl]boronic acid (80% w/w; 132 mg), triphenylphosphine (6.7 mg) and PdCl₂(PPh₃)₂ (18.3 mg). The mixture was heated to reflux for 2 h. The reaction mixture was filtered and the solvent was removed in vacuum. Silicagel chromatography followed by aminophase silicagel chromatography gave a solid that was triturated with a mixture of ethyl acetate and hexane to give 80 mg of the title compound.

¹H-NMR (400 MHz, Pyr-d₅), δ [ppm]=1.91-2.01 (2H), 2.05-2.16 (2H), 2.75 (1H), 2.93-3.06 (2H), 3.34 (3H), 3.40 (1H), 3.45-3.55 (1H), 3.59-3.75 (2H), 3.78-3.93 (3H), 4.05-4.17 (2H), 4.42-4.58 (2H), 4.79 (1H), 6.81 (1H), 7.04 (1H), 7.92 (1H), 8.03 (1H), 8.27 (1H), 8.43 (1H).

### Example III-038

### N-Ethyl-N-(2-methoxyethyl)-2-{6-[(3R)-morpholin-3-ylmethoxy]imidazo[1,2-b]pyridazin-3-yl}furo[3,2-c]pyridin-4-amine

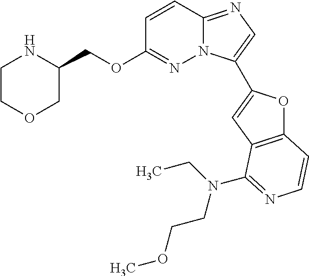 [SMILES: CCN(CCOC)c1nccc2oc(-c3cnc4ccc(OC[C@H]5COCCN5)nn34)cc12]

To a stirred solution of 3-bromo-6-[(3R)-morpholin-3-ylmethoxy]imidazo[1,2-b]pyridazine (100 mg) in 1-propanol (5 mL) was added 2M potassium carbonate solution (0.48 mL), crude {4-[Ethyl(2-methoxyethyl)amino]furo[3,2-c]pyridin-2-yl}boronic acid (80% w/w; 278 mg), triphenylphosphine (8.4 mg) and PdCl₂(PPh₃)₂ (22.9 mg). The mixture was heated to reflux for 2 h. The reaction mixture was filtered and the solvent was removed in vacuum. Silicagel chromatography followed by aminophase silicagel chromatography gave a solid that was triturated with a mixture of ethyl acetate and hexane to give 80 mg of the title compound.

¹H-NMR (400 MHz, Pyr-d₅), δ [ppm]=1.42 (3H), 2.76 (1H), 2.91-3.06 (2H), 3.36 (3H), 3.43-3.52 (1H), 3.58-3.72 (2H), 3.73-3.82 (2H), 3.84-3.96 (3H), 4.03 (2H), 4.16 (1H), 4.45 (2H), 6.82 (1H), 7.03 (1H), 7.75 (1H), 8.05 (1H), 8.25 (1H), 8.43 (1H).

### Example III-039

### N-(2-tert-Butoxyethyl)-N-ethyl-2-{6-[(3S)-morpholin-3-ylmethoxy]imidazo-[1,2-b]pyridazin-3-yl}furo[3,2-c]pyridin-4-amine

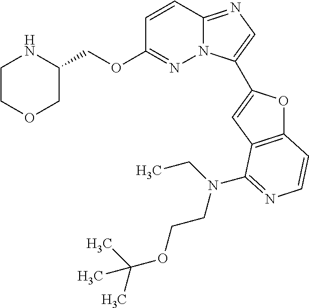 [SMILES: CCN(CCOC(C)(C)C)c1nccc2oc(-c3cnc4ccc(OC[C@@H]5COCCN5)nn34)cc12]

To a stirred solution of 3-bromo-6-[(3S)-morpholin-3-ylmethoxy]imidazo[1,2-b]pyridazine (80 mg) in 1-propanol (8.5 mL) was added 2M potassium carbonate solution (0.38 mL), crude {4-[(2-tert-butoxyethyl)(ethyl)amino]furo[3,2-c]pyridin-2-yl}boronic acid (50% w/w; 313 mg), triphenylphosphine (6.7 mg) and PdCl₂(PPh₃)₂ (18.3 mg). The mixture was heated to reflux for 2 h. The reaction mixture was filtered and the solvent was removed in vacuum. Silicagel chromatography followed by aminophase silicagel chromatography gave a solid that was triturated with a mixture of ethyl acetate and hexane to give 70 mg of the title compound.

¹H-NMR (300 MHz, Pyr-d₅), δ [ppm]=2.28 (9H), 2.56 (3H), 3.86 (1H), 4.00-4.19 (2H), 4.59 (1H), 4.68-4.84 (2H), 4.88-5.01 (3H), 5.07 (2H), 5.11-5.20 (2H), 5.28 (1H), 5.51-5.66 (2H), 7.93 (1H), 8.13 (1H), 8.91 (1H), 9.16 (1H), 9.37 (1H), 9.53 (1H).

### Example III-040

### 2-[Ethyl(2-{6-[(3S)-morpholin-3-ylmethoxy]imidazo[1,2-b]pyridazin-3-yl}furo[3,2-c]pyridin-4-yl)amino]ethanol

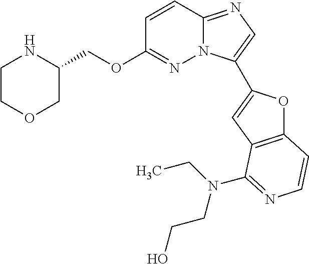 [SMILES: CCN(CCO)c1nccc2oc(-c3cnc4ccc(OC[C@@H]5COCCN5)nn34)cc12]

To a stirred solution of N-(2-tert-butoxyethyl)-N-ethyl-2-{6-[(3S)-morpholin-3-ylmethoxy]imidazo[1,2-b]pyridazin-3-yl}furo[3,2-c]pyridin-4-amine (20 mg) in Ethanol (4 mL) was added hydrochloric acid (0.40 mL; c=2.0 mol/L). The mixture was stirred at 40° C. for 2 h. Further hydrochloric acid (0.50 mL; c=4.0 mol/L) was added. The mixture was stirred at room temperature for 16 h. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was triturated with ethyl acetate to give 15 mg of the title compound.

¹H-NMR (400 MHz, DMSO-d₆, detected signals), δ [ppm]=1.23 (3H), 2.72-2.87 (2H), 3.12-3.21 (1H), 3.28-3.33 (1H), 3.40 (1H), 3.63-3.78 (7H), 3.81-3.88 (1H), 4.34 (2H), 4.74-5.18 (1H), 6.89 (1H), 7.00 (1H), 7.66 (1H), 7.92 (1H), 8.08 (1H), 8.14 (1H).

### Example III-041

### N-Ethyl-N-(2-methoxyethyl)-2-[6-(piperidin-2-ylmethoxy)imidazo[1,2-b]pyridazin-3-yl]furo[3,2-c]pyridin-4-amine

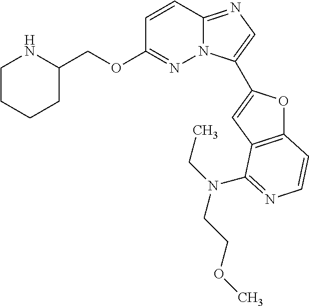 [SMILES: CCN(CCOC)c1nccc2oc(-c3cnc4ccc(OCC5CCCCN5)nn34)cc12]

To a stirred solution of 3-bromo-6-(piperidin-2-ylmethoxy)imidazo[1,2-b]pyridazine (100 mg) in 1-propanol (5 mL) was added 2M potassium carbonate solution (0.48 mL), crude {4-[Ethyl(2-methoxyethyl)amino]furo[3,2-c]pyridin-2-yl}boronic acid (82% w/w; 207 mg), triphenylphosphine (8.4 mg) and PdCl₂(PPh₃)₂ (22.6 mg). The mixture was heated to reflux for 2 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave 100 mg of the title compound.

¹H-NMR (400 MHz, DMSO-d₆, detected signals), δ [ppm]=1.10-1.21 (1H), 1.25 (3H), 1.28-1.37 (2H), 1.52 (1H), 1.63-1.82 (2H), 2.51-2.58 (1H), 2.86-2.99 (2H), 3.26 (3H), 3.56-3.63 (2H), 3.76 (2H), 3.83 (2H), 4.19-4.36 (2H), 6.91 (1H), 7.01 (1H), 7.65 (1H), 7.93 (1H), 8.08 (1H), 8.14 (1H).

### Example III-042

### 6-[3-(Methylsulfonyl)propoxy]-3-[4-(morpholin-4-yl)furo[3,2-c]pyridin-2-yl]imidazo[1,2-b]pyridazine

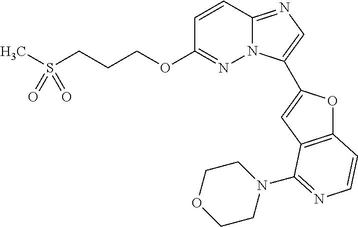 [SMILES: CS(=O)(=O)CCCOc1ccc2ncc(-c3cc4c(N5CCOCC5)nccc4o3)n2n1]

To a stirred suspension of 3-(methylsulfonyl)propan-1-ol (79 mg) in anhydrous THF (6 mL) was added sodium hydride (60% w/w in oil; 38 mg) at 0° C. and the mixture was stirred at room temperature for 30 minutes. 6-chloro-3-[4-(morpholin-4-yl)furo[3,2-c]pyridin-2-yl]imidazo[1,2-b]pyridazine (120 mg) was added and the mixture was heated to reflux for 2 hours. Water was added and the mixture was extracted with ethyl acetate. The organic phase was dried (sodium sulfate) and the solvent was removed in vacuum. Aminophase silicagel chromatography gave a solid that was triturated with a mixture of dichloromethane and hexane to give 34 mg of the title compound.

¹H-NMR (300 MHz, DMSO-d₆), δ [ppm]=2.18-2.35 (2H), 3.02 (3H), 3.30-3.39 (2H), 3.60-3.70 (4H), 3.72-3.84 (4H), 4.58 (2H), 7.02 (1H), 7.11 (1H), 7.64 (1H), 8.01 (1H), 8.12 (1H), 8.16 (1H).

### Example III-043

### 3-[4-(4-Methylpiperazin-1-yl)furo[3,2-c]pyridin-2-yl]-6-[3-(methylsulfonyl)-propoxy]imidazo[1,2-b]pyridazine

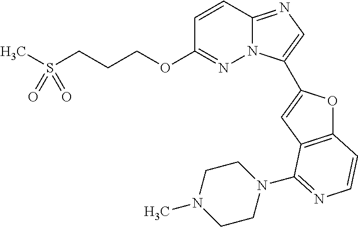 [SMILES: CN1CCN(c2nccc3oc(-c4cnc5ccc(OCCCS(C)(=O)=O)nn45)cc23)CC1]

To a stirred suspension of 3-(methylsulfonyl)propan-1-ol (115 mg) in anhydrous THF (4.5 mL) and anhydrous DMF (0.5 mL) was added sodium hydride (60% w/w in oil; 37 mg) at 0° C. and the mixture was stirred at room temperature for 30 minutes. 6-chloro-3-[4-(4-methylpiperazin-1-yl)furo[3,2-c]pyridin-2-yl]imidazo[1,2-b]pyridazine (140 mg) was added and the mixture was stirred at room temperature for 16 hours. Water was added and the mixture was extracted with ethyl acetate. The organic phase was dried (sodium sulfate) and the solvent was removed in vacuum. Silicagel chromatography gave a solid that was triturated with a mixture of dichloromethane and cyclohexane to give 110 mg of the title compound.

¹H-NMR (400 MHz, DMSO-d₆), δ [ppm]=2.19-2.33 (5H), 2.48 (4H), 3.01 (3H), 3.30-3.37 (2H), 3.62-3.75 (4H), 4.59 (2H), 7.02 (1H), 7.05-7.09 (1H), 7.64 (1H), 7.99 (1H), 8.12 (1H), 8.16 (1H).

### Example III-044

### 6-[3-(Methylsulfonyl)propoxy]-3-[4-(pyrrolidin-1-yl)furo[3,2-c]pyridin-2-yl]imidazo[1,2-b]pyridazine

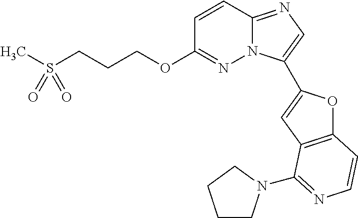 [SMILES: CS(=O)(=O)CCCOc1ccc2ncc(-c3cc4c(N5CCCC5)nccc4o3)n2n1]

To a stirred solution of 3-Bromo-6-[3-(methylsulfonyl)propoxy]imidazo[1,2-b]pyridazine (150 mg) in 1-propanol (15 mL) was added 2M potassium carbonate solution (0.67 mL), crude [4-(pyrrolidin-1-yl)furo[3,2-c]pyridin-2-yl]boronic acid (25% w/w; 1040 mg), triphenylphosphine (11.8 mg) and PdCl₂(PPh₃)₂ (32.1 mg). The mixture was heated to reflux for 1 h. The reaction mixture was filtered, a half-saturated solution of sodium bicarbonate was added and the mixture was extracted with a mixture of dichloromethane and methanol. The solvent was removed in vacuum. Silicagel chromatography gave a solid that was triturated with warm ethanol to give 40 mg of the title compound.

¹H-NMR (300 MHz, DMSO-d₆), δ [ppm]=1.90-2.01 (4H), 2.19-2.35 (2H), 3.02 (3H), 3.33 (2H), 3.62-3.76 (4H), 4.54 (2H), 6.86 (1H), 6.98 (1H), 7.73 (1H), 7.91 (1H), 8.06 (1H), 8.13 (1H).

### Example III-045

### 6-[3-(methylsulfonyl)propoxy]-3-[4-(piperidin-1-yl)furo[3,2-c]pyridin-2-yl]imidazo[1,2-b]pyridazine

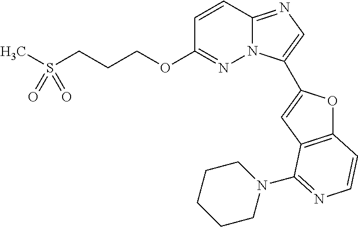 [SMILES: CS(=O)(=O)CCCOc1ccc2ncc(-c3cc4c(N5CCCCC5)nccc4o3)n2n1]

To a stirred solution of 3-Bromo-6-[3-(methylsulfonyl)propoxy]imidazo[1,2-b]pyridazine (150 mg) in 1-propanol (15 mL) was added 2M potassium carbonate solution (0.67 mL), crude [4-(piperidin-1-yl)furo[3,2-c]pyridin-2-yl]boronic acid (166 mg), triphenylphosphine (11.8 mg) and PdCl₂(PPh₃)₂ (32.1 mg). The mixture was heated to reflux for 2 h. The reaction mixture was filtered, a half-saturated solution of sodium bicarbonate was added and the mixture was extracted with a mixture of dichloromethane and methanol. The solvent was removed in vacuum. Silicagel chromatography gave a solid that was triturated with warm ethyl acetate to give 160 mg of the title compound.

¹H-NMR (300 MHz, DMSO-d₆), δ [ppm]=1.64 (6H), 2.19-2.34 (2H), 3.01 (3H), 3.30-3.37 (2H), 3.66 (4H), 4.56 (2H), 6.96-7.04 (2H), 7.60 (1H), 7.96 (1H), 8.10 (1H), 8.15 (1H).

### Example III-046

### (3R)—N,N-Dimethyl-1-(2-{6-[3-(methylsulfonyl)propoxy]imidazo[1,2-b]pyridazin-3-yl}furo[3,2-c]pyridin-4-yl)pyrrolidin-3-amine

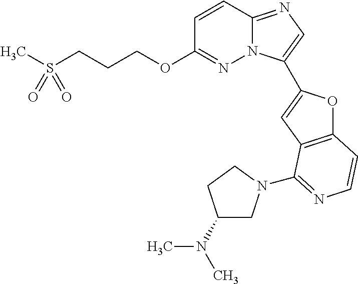 [SMILES: CN(C)[C@@H]1CCN(c2nccc3oc(-c4cnc5ccc(OCCCS(C)(=O)=O)nn45)cc23)C1]

To a stirred solution of 3-Bromo-6-[3-(methylsulfonyl)propoxy]imidazo[1,2-b]pyridazine (100 mg) in 1-propanol (9 mL) was added 2M potassium carbonate solution (0.45 mL), crude {4-[(3R)-3-(dimethylamino)pyrrolidin-1-yl]furo[3,2-c]pyridin-2-yl}boronic acid (65% w/w; 253 mg), triphenylphosphine (7.8 mg) and PdCl₂(PPh₃)₂ (21.0 mg). The mixture was heated to reflux for 1.5 h. The reaction mixture was filtered, a half-saturated solution of sodium bicarbonate was added and the mixture was extracted with a mixture of dichloromethane and methanol. The solvent was removed in vacuum. Silicagel chromatography gave a solid that was triturated with a mixture of ethyl acetate and hexane to give 94 mg of the title compound.

¹H-NMR (400 MHz, DMSO-d6), δ [ppm]=1.75-1.88 (1H), 2.12-2.28 (9H), 2.73-2.83 (1H), 3.02 (3H), 3.29-3.35 (2H), 3.40 (1H), 3.58-3.72 (1H), 3.82-3.99 (2H), 4.52 (2H), 6.87 (1H), 6.97 (1H), 7.67 (1H), 7.91 (1H), 8.06 (1H), 8.12 (1H).

### Example III-047

### N-methyl-2-{6-[3-(methylsulfonyl)propoxy]imidazo[1,2-b]pyridazin-3-yl}-N-[3-(pyrrolidin-1-yl)propyl]furo[3,2-c]pyridin-4-amine

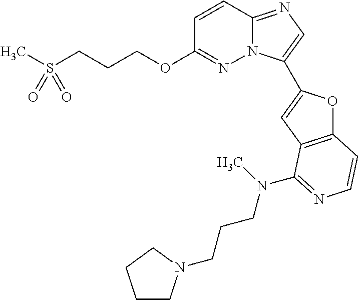 [SMILES: CN(CCCN1CCCC1)c1nccc2oc(-c3cnc4ccc(OCCCS(C)(=O)=O)nn34)cc12]

To a stirred solution of 3-Bromo-6-[3-(methylsulfonyl)propoxy]imidazo[1,2-b]pyridazine (100 mg) in 1-propanol (9 mL) was added 2M potassium carbonate solution (0.45 mL), crude (4-{methyl[3-(pyrrolidin-1-yl)propyl]amino}furo[3,2-c]pyridin-2-yl)boronic acid (52% w/w; 349 mg), triphenylphosphine (7.8 mg) and PdCl₂(PPh₃)₂ (21.0 mg). The mixture was heated to reflux for 1.5 h. The reaction mixture was filtered, a half-saturated solution of sodium bicarbonate was added and the mixture was extracted with a mixture of dichloromethane and methanol. The solvent was removed in vacuum. Silicagel chromatography gave a solid that was triturated with methanol to give 62 mg of the title compound.

¹H-NMR (300 MHz, DMSO-d₆, detected signals), δ [ppm]=1.58 (4H), 1.76 (2H), 2.18-2.43 (8H), 3.01 (3H), 3.34 (2H), 3.74 (2H), 4.55 (2H), 6.89 (1H), 7.00 (1H), 7.73 (1H), 7.93 (1H), 8.08 (1H), 8.15 (1H).

### Example III-048

3-[4-(4-tert-Butylpiperazin-1-yl)furo[3,2-c]pyridin-2-yl]-6-[3-(methylsulfonyl)-propoxy]imidazo[1,2-b]pyridazine

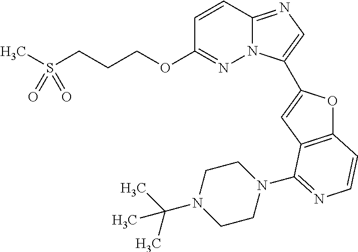 [SMILES: CC(C)(C)N1CCN(c2nccc3oc(-c4cnc5ccc(OCCCS(C)(=O)=O)nn45)cc23)CC1]

To a stirred solution of 3-Bromo-6-[3-(methylsulfonyl)propoxy]imidazo[1,2-b]pyridazine (150 mg) in 1-propanol (15 mL) was added 2M potassium carbonate solution (0.67 mL), crude [4-(4-tert-butylpiperazin-1-yl)furo[3,2-c]pyridin-2-yl]boronic acid (55% w/w; 371 mg), triphenylphosphine (11.8 mg) and PdCl₂(PPh₃)₂ (32.1 mg). The mixture was heated to reflux for 2 h. A mixture of dichloromethane and methanol was added. The reaction mixture was filtered, a half-saturated solution of sodium bicarbonate was added and the mixture was extracted with a mixture of dichloromethane and methanol. The solvent was removed in vacuum. Silicagel chromatography followed by aminophase silicagel chromatography gave a solid that was triturated ethyl acetate to give 85 mg of the title compound.

¹H-NMR (300 MHz, DMSO-d₆), δ [ppm]=1.03 (9H), 2.20-2.34 (2H), 2.58-2.71 (4H), 3.00 (3H), 3.30-3.37 (2H), 3.64 (4H), 4.59 (2H), 6.96-7.07 (2H), 7.64 (1H), 7.98 (1H), 8.10 (1H), 8.15 (1H).

### Example III-049

### tert-Butyl 4-(2-{6-[3-(methylsulfonyl)propoxy]imidazo[1,2-b]pyridazin-3-yl}furo[3,2-c]pyridin-4-yl)piperazine-1-carboxylate

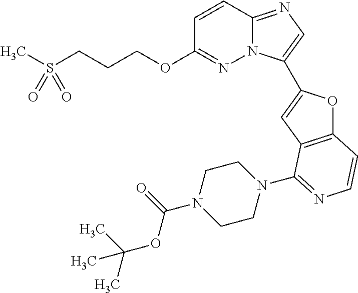 [SMILES: CC(C)(C)OC(=O)N1CCN(c2nccc3oc(-c4cnc5ccc(OCCCS(C)(=O)=O)nn45)cc23)CC1]

To a stirred solution of 3-Bromo-6-[3-(methylsulfonyl)propoxy]imidazo[1,2-b]pyridazine (120 mg) in 1-propanol (12 mL) was added 2M potassium carbonate solution (0.54 mL), crude {4-[4-(tert-butoxycarbonyl)piperazin-1-yl]furo[3,2-c]pyridin-2-yl}boronic acid (90% w/w; 277 mg), triphenylphosphine (9.4 mg) and PdCl₂(PPh₃)₂ (25.7 mg). The mixture was heated to reflux for 2 h. A mixture of dichloromethane and methanol was added and the reaction mixture was filtered. A half-saturated solution of sodium bicarbonate was added and the mixture was extracted with a mixture of dichloromethane and methanol. The solvent was removed in vacuum. Aminophase silicagel chromatography followed by silicagel chromatography gave a solid that was triturated ethyl acetate to give 150 mg of the title compound.

¹H-NMR (400 MHz, CHLOROFORM-d), δ [ppm]=1.50 (9H), 2.43-2.58 (2H), 3.03 (3H), 3.31 (2H), 3.58-3.68 (4H), 3.76 (4H), 4.66 (2H), 6.80 (1H), 7.00 (1H), 7.53 (1H), 7.93 (1H), 8.08 (1H), 8.15 (1H).

### Example III-050

### N-Ethyl-N-(2-methoxyethyl)-2-{6-[3-(methylsulfonyl)propoxy]imidazo[1,2-b]pyridazin-3-yl}furo[3,2-c]pyridin-4-amine

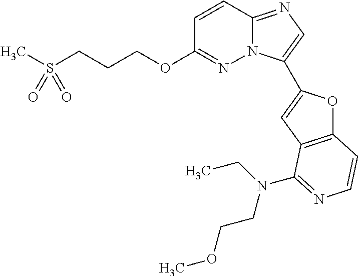 [SMILES: CCN(CCOC)c1nccc2oc(-c3cnc4ccc(OCCCS(C)(=O)=O)nn34)cc12]

To a stirred solution of 3-Bromo-6-[3-(methylsulfonyl)propoxy]imidazo[1,2-b]pyridazine (80 mg) in 1-propanol (8 mL) was added 2M potassium carbonate solution (0.36 mL), crude {4-[Ethyl(2-methoxyethyl)amino]furo[3,2-c]pyridin-2-yl}boronic acid (80% w/w; 158 mg), triphenylphosphine (6.2 mg) and PdCl₂(PPh₃)₂ (17.1 mg). The mixture was heated to reflux for 4 h. A mixture of dichloromethane and methanol was added and the mixture was filtered through an aminophase silicagel column. The solvent was removed in vacuum. Silicagel chromatography gave a solid that was triturated ethyl acetate to give 90 mg of the title compound.

¹H-NMR (300 MHz, DMSO-d₆), δ [ppm]=1.22 (3H), 2.17-2.32 (2H), 3.02 (3H), 3.27 (3H), 3.30-3.37 (2H), 3.54-3.64 (2H), 3.67-3.88 (4H), 4.55 (2H), 6.90 (1H), 7.00 (1H), 7.60 (1H), 7.92 (1H), 8.08 (1H), 8.15 (1H).

### Example III-051

### 1-[4-(2-{6-[3-(Methylsulfonyl)propoxy]imidazo[1,2-b]pyridazin-3-yl}furo[3,2-c]pyridin-4-yl)piperazin-1-yl]ethanone

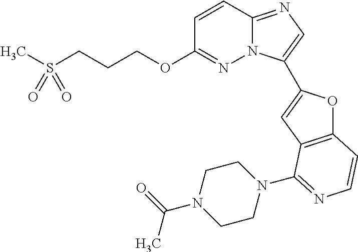 [SMILES: CC(=O)N1CCN(c2nccc3oc(-c4cnc5ccc(OCCCS(C)(=O)=O)nn45)cc23)CC1]

To a stirred suspension of tert-butyl 4-(2-{6-[3-(methylsulfonyl)propoxy]imidazo[1,2-b]pyridazin-3-yl}furo[3,2-c]pyridin-4-yl)piperazine-1-carboxylate (135 mg) in dichloromethane (21 mL) was added TFA (0.47 mL). The mixture was stirred at room temperature for 16 h. A saturated solution of potassium carbonate was added and the mixture was extracted with a mixture of dichloromethane and methanol. The organic phase was dried (sodium sulfate) and the solvent was removed in vacuum. Aminophase silicagel chromatography gave a solid that was triturated with ethanol to give 100 mg of 6-[3-(methylsulfonyl)propoxy]-3-[4-(piperazin-1-yl)furo[3,2-c]pyridin-2-yl]imidazo[1,2-b]pyridazine, that were directly used for the next step.

To a stirred solution of 6-[3-(methylsulfonyl)propoxy]-3-[4-(piperazin-1-yl)furo[3,2-c]pyridin-2-yl]imidazo[1,2-b]pyridazine (80 mg) in THF (5 mL) and pyridine (0.04 mL) was added acetic anhydride (0.025 mL), and the mixture was stirred for 70 h. Further pyridine (2.0 mL), acetic anhydride (0.5 mL) and DMF (0.5 mL) were added and the mixture was stirred for 2 h. The solvent was removed in vacuum. Silicagel chromatography gave a solid that was triturated with ethanol to give 40 mg of the title compound.

¹H-NMR (300 MHz, Pyr-d5), δ [ppm]=2.20 (3H), 2.60-2.74 (2H), 3.32 (3H), 3.63-3.73 (4H), 3.87-4.04 (6H), 4.75 (2H), 6.84 (1H), 7.19 (1H), 7.77 (1H), 8.10 (1H), 8.30 (1H), 8.47 (1H).

### Example III-052

### N-(2-Methoxyethyl)-N-methyl-2-{6-[3-(methylsulfonyl)propoxy]imidazo[1,2-b]pyridazin-3-yl}furo[3,2-c]pyridin-4-amine

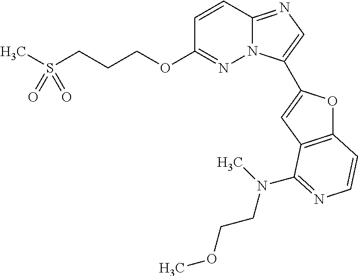 [SMILES: COCCN(C)c1nccc2oc(-c3cnc4ccc(OCCCS(C)(=O)=O)nn34)cc12]

To a stirred solution of 3-Bromo-6-[3-(methylsulfonyl)propoxy]imidazo[1,2-b]pyridazine (80 mg) in 1-propanol (8 mL) was added 2M potassium carbonate solution (0.36 mL), crude {4-[(2-methoxyethyl)(methyl)amino]furo[3,2-c]pyridin-2-yl}boronic acid (80% w/w; 150 mg), triphenylphosphine (6.3 mg) and PdCl₂(PPh₃)₂ (17.1 mg). The mixture was heated to reflux for 4 h. The reaction mixture was filtered through an aminophase silicagel column and the solvent was removed in vacuum. Silicagel chromatography gave a solid that was triturated with 2-propanol to give 80 mg of the title compound.

¹H-NMR (400 MHz, Pyr-d₅), δ [ppm]=2.56-2.70 (2H), 3.22 (3H), 3.32 (3H), 3.46 (3H), 3.63-3.69 (2H), 3.71 (2H), 3.99 (2H), 4.66 (2H), 6.78 (1H), 7.02 (1H), 7.81 (1H), 8.04 (1H), 8.23 (1H), 8.40 (1H).

### Example III-053

### 3-{4-[(2R,6S)-2,6-Dimethylmorpholin-4-yl]furo[3,2-c]pyridin-2-yl}-6-[3-(methyl-sulfonyl)propoxy]imidazo[1,2-b]pyridazine

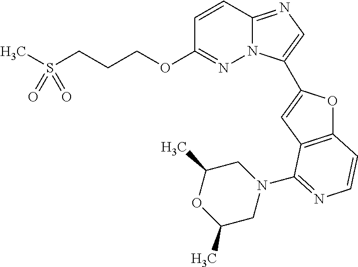 [SMILES: C[C@@H]1CN(c2nccc3oc(-c4cnc5ccc(OCCCS(C)(=O)=O)nn45)cc23)C[C@H](C)O1]

To a stirred solution of 3-Bromo-6-[3-(methylsulfonyl)propoxy]imidazo[1,2-b]pyridazine (100 mg) in 1-propanol (8.5 mL) was added 2M potassium carbonate solution (0.45 mL), crude {4-[(2R,6S)-2,6-dimethylmorpholin-4-yl]furo[3,2-c]pyridin-2-yl}boronic acid (75% w/w; 220 mg), triphenylphosphine (7.8 mg) and PdCl₂(PPh₃)₂ (21.0 mg). The mixture was heated to reflux for 1 h. The warm mixture was filtered, the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with a mixture of dichloromethane and hexane to give 46 mg of the title compound.

¹H-NMR (400 MHz, DMSO-d₆), δ [ppm]=1.11-1.19 (6H), 2.19-2.28 (2H), 2.71 (2H), 3.01 (3H), 3.30-3.37 (2H), 3.66-3.77 (2H), 4.24 (2H), 4.60 (2H), 7.02 (1H), 7.08 (1H), 7.66 (1H), 8.00 (1H), 8.12 (1H), 8.16 (1H).

### Example III-054

### 3-[Methyl(2-{6-[3-(methylsulfonyl)propoxy]imidazo[1,2-b]pyridazin-3-yl}furo[3,2-c]pyridin-4-yl)amino]propan-1-ol

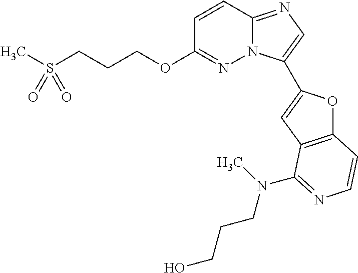 [SMILES: CN(CCCO)c1nccc2oc(-c3cnc4ccc(OCCCS(C)(=O)=O)nn34)cc12]

To a stirred solution of N-(3-{[tert-butyl(dimethyl)silyl]oxy}propyl)-N-methyl-2-{6-[3-(methylsulfonyl)propoxy]imidazo[1,2-b]pyridazin-3-yl}furo[3,2-c]pyridin-4-amine (130 mg) in THF (12 mL) was added a solution of TBAF in THF (0.45 mL; c=1.0 mol/L). The mixture was stirred at room temperature for 4 h. A half-saturated solution of sodium bicarbonate was added and the mixture was extracted with a mixture of ethyl acetate and methanol. The organic phase was dried (sodium sulfate) and the solvent was removed in vacuum. Silicagel chromatography gave a solid that was triturated with a mixture of dichloromethane and hexane to give 57 mg of the title compound.

¹H-NMR (300 MHz, DMSO-d₆, detected signals), δ [ppm]=1.78 (2H), 2.17-2.32 (2H), 3.01 (3H), 3.27 (3H), 3.47 (2H), 3.74 (2H), 4.49-4.67 (3H), 6.90 (1H), 7.00 (1H), 7.73 (1H), 7.93 (1H), 8.08 (1H), 8.15 (1H).

### Example III-055

### N,N-Dimethyl-1-(2-{6-[3-(methylsulfonyl)propoxy]imidazo[1,2-b]pyridazin-3-yl}furo[3,2-c]pyridin-4-yl)piperidin-4-amine

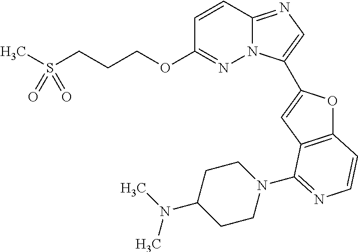 [SMILES: CN(C)C1CCN(c2nccc3oc(-c4cnc5ccc(OCCCS(C)(=O)=O)nn45)cc23)CC1]

To a stirred solution of 3-Bromo-6-[3-(methylsulfonyl)propoxy]imidazo[1,2-b]pyridazine (60 mg) in 1-propanol (6 mL) was added 2M potassium carbonate solution (0.27 mL), crude {4-[4-(dimethylamino)piperidin-1-yl]furo[3,2-c]pyridin-2-yl}boronic acid (134 mg), triphenylphosphine (4.7 mg) and PdCl₂(PPh₃)₂ (12.9 mg). The mixture was heated to reflux for 4 h. The reaction mixture was filtered through an aminophase silicagel column and the solvent was removed in vacuum. Silicagel chromatography gave a solid that was triturated with 2-propanol to give 50 mg of the title compound.

¹H-NMR (300 MHz, DMSO-d₆), δ [ppm]=1.34-1.54 (2H), 1.87 (2H), 2.18 (6H), 2.21-2.39 (3H), 2.91-3.07 (5H), 3.32-3.39 (2H), 4.35 (2H), 4.56 (2H), 6.96-7.04 (2H), 7.58 (1H), 7.96 (1H), 8.09 (1H), 8.14 (1H).

### Example III-056

### 6-[3-(Methylsulfonyl)propoxy]-3-[4-(4-phenylpiperazin-1-yl)furo[3,2-c]pyridin-2-yl]imidazo[1,2-b]pyridazine

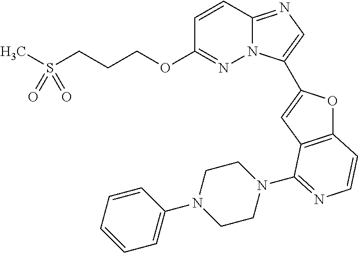 [SMILES: CS(=O)(=O)CCCOc1ccc2ncc(-c3cc4c(N5CCN(c6ccccc6)CC5)nccc4o3)n2n1]

To 167 mg (0.5 mmol) 3-bromo-6-[3-(methylsulfonyl)propoxy]imidazo-[1,2-b]pyridazine in 6 mL propan-1-ol were added 242 mg (0.75 mmol) [4-(4-phenylpiperazin-1-yl)furo[3,2-c]pyridin-2-yl]boronic acid, 23 mg (20 μmol) tetrakis(triphenylphosphin)-palladium(0), and 0.75 mL of a 2 M aqueous solution of sodium carbonate. The mixture was stirred at 100° C. for 5 h.

Water was added. The mixture was concentrated. 30 mL of a mixture of water a methanol (1:1) was added. The precipitate was filtered off, washed with methanol and dried in vacuum. The obtained crude material was digested in a mixture of dichloromethane and methanol (1:1) to give 157 mg of the title compound.

¹H-NMR (300 MHz, DMSO-d₆), δ [ppm]=2.26-2.38 (2H), 2.99 (3H), 3.33-3.42 (4H), 3.83 (4H), 4.64 (2H), 6.77 (1H), 6.97-7.07 (3H), 7.12 (1H), 7.18-7.26 (2H), 7.71 (1H), 8.03 (1H), 8.14 (1H), 8.18 (1H).

LC-MS (Method 14): Rₜ=0.57 min; MS (ESIpos) m/z=409 [M+H]+.

### Example III-057

### [1-(2-{6-[3-(Methylsulfonyl)propoxy]imidazo[1,2-b]pyridazin-3-yl}furo[3,2-c]pyridin-4-yl)piperidin-4-yl]methanol

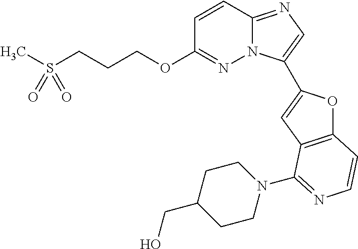 [SMILES: CS(=O)(=O)CCCOc1ccc2ncc(-c3cc4c(N5CCC(CO)CC5)nccc4o3)n2n1]

To a stirred solution of 3-{4-[4-({[tert-butyl(dimethyl)silyl]oxy}methyl)piperidin-1-yl]furo[3,2-c]pyridin-2-yl}-6-[3-(methylsulfonyl)propoxy]imidazo[1,2-b]pyridazine (130 mg) in THF (10 mL) was added a solution of TBAF in THF (0.54 mL; c=1.0 mol/L). The mixture was stirred at room temperature for 48 h. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with ethyl acetate to give 70 mg of the title compound.

¹H-NMR (300 MHz, DMSO-d₆), δ [ppm]=1.13-1.33 (2H), 1.58-1.85 (3H), 2.18-2.34 (2H), 2.89-3.08 (5H), 3.24-3.29 (2H), 3.31-3.37 (2H), 4.37 (2H), 4.46 (1H), 4.52 (2H), 6.95-7.02 (2H), 7.56 (1H), 7.95 (1H), 8.07 (1H), 8.13 (1H).

### Example III-058

### 3-{4-[(3R)-3-Methylmorpholin-4-yl]furo[3,2-c]pyridin-2-yl}-6-[3-(methyl-sulfonyl)propoxy]imidazo[1,2-b]pyridazine

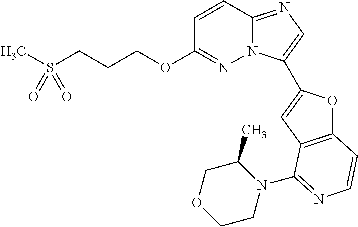 [SMILES: C[C@@H]1COCCN1c1nccc2oc(-c3cnc4ccc(OCCCS(C)(=O)=O)nn34)cc12]

To 123 mg (0.37 mmol) 3-bromo-6-[3-(methylsulfonyl)propoxy]imidazo-[1,2-b]pyridazine in 6 mL propan-1-ol were added 323 mg (0.74 mmol) {4-[(3R)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Water was added. The mixture was extracted with dichloromethane. The organic layer was dried over sodium sulfate and evaporated. The obtained crude product was digested in methanol to give 122 mg of the title compound.

¹H-NMR (400 MHz, DMSO-d6), δ [ppm]=1.25 (3H), 2.20-2.33 (2H), 3.02 (3H), 3.31-3.43 (2H), 3.50-3.62 (1H), 3.67-3.78 (2H), 3.90-3.99 (1H), 4.11 (1H), 4.44-4.66 (3H), 6.98-7.07 (2H), 7.65 (1H), 8.00 (1H), 8.11 (1H), 8.16 (1H).

LC-MS (Method 14): Rₜ=0.72 min; MS (ESIpos) m/z=472 [M+H]⁺.

### Example III-059

### 3-{4-[(3S)-3-Methyl morpholin-4-yl]furo[3,2-c]pyridin-2-yl}-6-[3-(methyl-sulfonyl)propoxy]imidazo[1,2-b]pyridazine

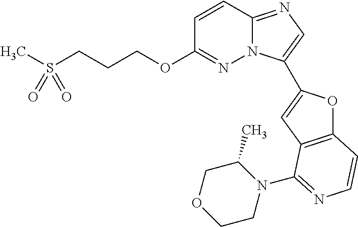 [SMILES: C[C@H]1COCCN1c1nccc2oc(-c3cnc4ccc(OCCCS(C)(=O)=O)nn34)cc12]

To 123 mg (0.37 mmol) 3-bromo-6-[3-(methylsulfonyl)propoxy]imidazo-[1,2-b]pyridazine in 6 mL propan-1-ol were added 323 mg (0.74 mmol) {4-[(35)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Half-saturated brine was added. The mixture was extracted with dichloromethane. The organic layer was dried over sodium sulfate and evaporated. The obtained crude product was digested in methanol to give 122 mg of the title compound.

¹H-NMR (400 MHz, DMSO-d6), δ [ppm]=1.28 (3H), 2.30 (2H), 3.04 (3H), 3.33-3.38 (2H), 3.42 (1H), 3.59 (1H), 3.71-3.80 (2H), 3.98 (1H), 4.14 (1H), 4.49-4.68 (3H), 7.02-7.10 (2H), 7.69 (1H), 8.03 (1H), 8.14 (1H), 8.19 (1H).

LC-MS (Method 13): Rₜ=0.74 min; MS (ESIpos) m/z=472 [M+H]⁺.

### Example III-060

### 3-{4-[(2S)-2-Methyl morpholin-4-yl]furo[3,2-c]pyridin-2-yl}-6-[3-(methyl-sulfonyl)propoxy]imidazo[1,2-b]pyridazine

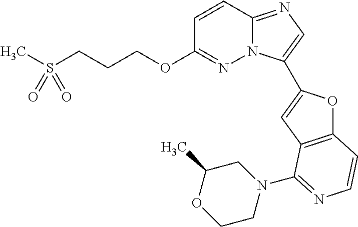 [SMILES: C[C@H]1CN(c2nccc3oc(-c4cnc5ccc(OCCCS(C)(=O)=O)nn45)cc23)CCO1]

To 123 mg (0.37 mmol) 3-bromo-6-[3-(methylsulfonyl)propoxy]imidazo-[1,2-b]pyridazine in 6 mL propan-1-ol were added 323 mg (0.74 mmol) {4-[(2S)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 18 h.

Water was added. The mixture was extracted with dichloromethane. The organic layer was dried over sodium sulfate and evaporated. The obtained crude product was digested in a mixture of DMSO and methanol to give 138 mg of the title compound.

¹H-NMR (400 MHz, DMSO-d6), δ [ppm]=1.19 (3H), 2.22-2.35 (2H), 2.80 (1H), 3.04 (3H), 3.13 (1H), 3.37 (2H), 3.61-3.77 (2H), 3.97 (1H), 4.21 (2H), 4.62 (2H), 7.05 (1H), 7.12 (1H), 7.67 (1H), 8.03 (1H), 8.15 (1H), 8.19 (1H).

LC-MS (Method 12): Rₜ=0.76 min; MS (ESIpos) m/z=472 [M+H]⁺.

### Example III-061

### (5R)-5-[({3-[4-(4-Methylpiperazin-1-yl)furo[3,2-c]pyridin-2-yl]imidazo[1,2-b]pyridazin-6-yl}oxy)methyl]pyrrolidin-2-one

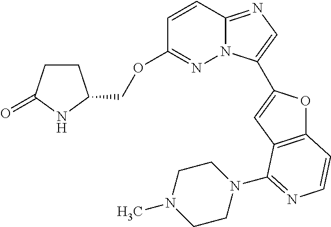 [SMILES: CN1CCN(c2nccc3oc(-c4cnc5ccc(OC[C@H]6CCC(=O)N6)nn45)cc23)CC1]

To a stirred suspension of (5R)-5-(hydroxymethyl)pyrrolidin-2-one (93.6 mg) in anhydrous THF (10 mL) and anhydrous DMF (1.0 mL) was added sodium hydride (60% w/w in oil; 57 g) at 0° C. and the mixture was stirred at room temperature for 30 minutes. 6-chloro-3-[4-(4-methylpiperazin-1-yl)furo[3,2-c]pyridin-2-yl]imidazo[1,2-b]pyridazine (150 mg) was added and the mixture was stirred at 60° C. for 16 hours. DMF (4 mL) was added and the mixture was stirred at 60° C. for further 2 hours. Water was added and the solvent was removed in vacuum. Silicagel chromatography gave a solid that dissolved in water and freeze dried to give 110 mg of the title compound.

¹H-NMR (400 MHz, DMSO-d₆), δ [ppm]=1.78-2.36 (7H), 3.60-3.70 (4H), 3.95-4.04 (1H), 4.27-4.35 (1H), 4.36-4.44 (1H), 6.98 (1H), 7.01-7.07 (1H), 7.60 (1H), 7.92 (1H), 7.97 (1H), 8.09 (1H), 8.13 (1H).

### Example III-062

### (5R)-5-[({3-[4-(Piperidin-1-yl)furo[3,2-c]pyridin-2-yl]imidazo[1,2-b]pyridazin-6-yl}oxy)methyl]pyrrolidin-2-one

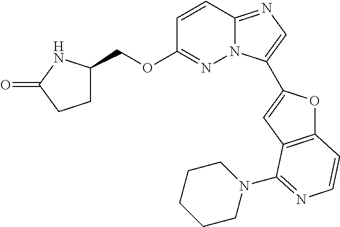 [SMILES: O=C1CC[C@H](COc2ccc3ncc(-c4cc5c(N6CCCCC6)nccc5o4)n3n2)N1]

To a stirred solution of (5R)-5-{[(3-bromoimidazo[1,2-b]pyridazin-6-yl)oxy]methyl}pyrrolidin-2-one (150 mg) in 1-propanol (16 mL) was added 2M potassium carbonate solution (0.72 mL), crude [4-(piperidin-1-yl)furo[3,2-c]pyridin-2-yl]boronic acid (177 mg), triphenylphosphine (12.6 mg) and PdCl₂(PPh₃)₂ (34.5 mg). The mixture was heated to reflux for 2 h. A mixture of dichloromethane and methanol was added and the mixture was filtered. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with ethyl acetate to give 150 mg of the title compound.

¹H-NMR (300 MHz, DMSO-d₆), δ [ppm]=1.63 (6H), 1.77-1.95 (1H), 2.09-2.35 (3H), 3.65 (4H), 3.99 (1H), 4.25-4.34 (1H), 4.37-4.48 (1H), 6.97-7.00 (1H), 7.02 (1H), 7.61 (1H), 7.92 (1H), 7.96 (1H), 8.10 (1H), 8.15 (1H).

### Example III-063

### (5R)-5-[({3-[4-(4-tert-Butylpiperazin-1-yl)furo[3,2-c]pyridin-2-yl]imidazo-[1,2-b]pyridazin-6-yl}oxy)methyl]pyrrolidin-2-one

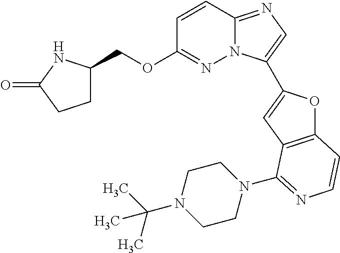 [SMILES: CC(C)(C)N1CCN(c2nccc3oc(-c4cnc5ccc(OC[C@H]6CCC(=O)N6)nn45)cc23)CC1]

To a stirred solution of (5R)-5-{[(3-bromoimidazo[1,2-b]pyridazin-6-yl)oxy]methyl}pyrrolidin-2-one (150 mg) in 1-propanol (16 mL) was added 2M potassium carbonate solution (0.72 mL), crude [4-(4-tert-butylpiperazin-1-yl)furo[3,2-c]pyridin-2-yl]boronic acid (399 mg), triphenylphosphine (12.6 mg) and PdCl₂(PPh₃)₂ (34.5 mg). The mixture was heated to reflux for 2 h. A mixture of dichloromethane and methanol was added and the mixture was filtered. A half-saturated solution of sodium bicarbonate was added and the mixture was extracted with a mixture of dichloromethane and methanol. The organic phase was dried (sodium sulfate) and the solvent was removed in vacuum. Silicagel chromatography followed by aminophase silicagel chromatography gave a solid that was triturated with ethyl acetate to give 140 mg of the title compound.

¹H-NMR (300 MHz, DMSO-d₆), δ [ppm]=1.02 (9H), 1.73-1.88 (1H), 2.08-2.33 (3H), 2.63 (4H), 3.61 (4H), 3.98 (1H), 4.23-4.32 (1H), 4.33-4.42 (1H), 6.94 (1H), 7.01 (1H), 7.55 (1H), 7.95 (1H), 8.00 (1H), 8.06 (1H), 8.11 (1H).

### Example III-064

### (5R)-5-{[(3-{4-[(3R)-3-(Dimethylamino)pyrrolidin-1-yl]furo[3,2-c]pyridin-2-yl}imidazo[1,2-b]pyridazin-6-yl)oxy]methyl}pyrrolidin-2-one

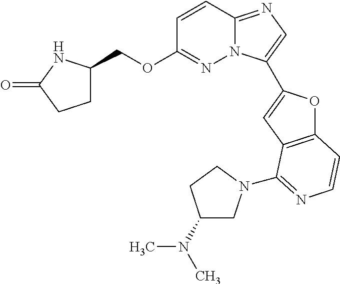 [SMILES: CN(C)[C@@H]1CCN(c2nccc3oc(-c4cnc5ccc(OC[C@H]6CCC(=O)N6)nn45)cc23)C1]

To a stirred solution of (5R)-5-{[(3-bromoimidazo[1,2-b]pyridazin-6-yl)oxy]methyl}pyrrolidin-2-one (100 mg) in 1-propanol (9 mL) was added 2M potassium carbonate solution (0.48 mL), crude {4-[(3R)-3-(dimethylamino)pyrrolidin-1-yl]furo[3,2-c]pyridin-2-yl}boronic acid (52% w/w; 340 mg), triphenylphosphine (8.4 mg) and PdCl₂(PPh₃)₂ (22.6 mg). The mixture was heated to reflux for 1 h. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 47 mg of the title compound.

¹H-NMR (400 MHz, Pyr-d5), δ [ppm]=1.86-2.19 (3H), 2.22-2.39 (7H), 2.41-2.52 (1H), 2.56-2.67 (1H), 2.73-2.87 (1H), 3.73 (1H), 3.82-3.93 (1H), 4.04-4.19 (2H), 4.26 (1H), 4.40-4.56 (2H), 6.78 (1H), 7.04 (1H), 7.82 (1H), 8.07 (1H), 8.29 (1H), 8.42 (1H), 9.09 (1H).

### Example III-065

### (5R)-5-{[(3-{4-[Methyl(1-methylpiperidin-4-yl)amino]furo[3,2-c]pyridin-2-yl}imidazo[1,2-b]pyridazin-6-yl)oxy]methyl}pyrrolidin-2-one

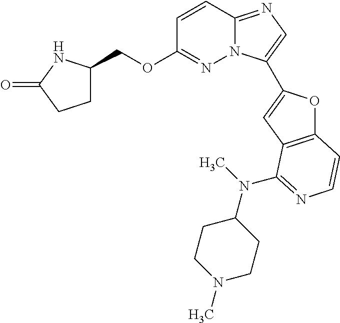 [SMILES: CN1CCC(N(C)c2nccc3oc(-c4cnc5ccc(OC[C@H]6CCC(=O)N6)nn45)cc23)CC1]

To a stirred solution of (5R)-5-{[(3-bromoimidazo[1,2-b]pyridazin-6-yl)oxy]-methyl}pyrrolidin-2-one (100 mg) in 1-propanol (10 mL) was added 2M potassium carbonate solution (0.48 mL), crude {4-[methyl(1-methylpiperidin-4-yl)amino]furo[3,2-c]pyridin-2-yl}boronic acid (26% w/w; 638 mg), triphenylphosphine (8.4 mg) and PdCl₂(PPh₃)₂ (22.6 mg). The mixture was heated to reflux for 1 h. Water was added and the mixture was extracted with a mixture of dichloromethane and methanol. The organic phase was dried (sodium sulfate) and the solvent was removed in vacuum. Silicagel chromatography gave 48 mg of the title compound.

¹H-NMR (400 MHz, Pyr-d5), δ [ppm]=1.65-1.76 (2H), 1.86-2.07 (5H), 2.16 (3H), 2.21-2.34 (1H), 2.36-2.48 (1H), 2.51-2.63 (1H), 2.78-2.91 (2H), 3.31 (3H), 4.17-4.28 (1H), 4.34-4.44 (1H), 4.54 (1H), 4.83-4.94 (1H), 6.75 (1H), 7.03 (1H), 7.78 (1H), 8.03 (1H), 8.24 (1H), 8.39 (1H), 9.11 (1H).

### Example III-066

### (5R)-5-{[(3-{4-[(2R)-2-({[tert-Butyl(dimethyl)silyl]oxy}methyl)pyrrolidin-1-yl]furo[3,2-c]pyridin-2-yl}imidazo[1,2-b]pyridazin-6-yl)oxy]methyl}pyrrolidin-2-one

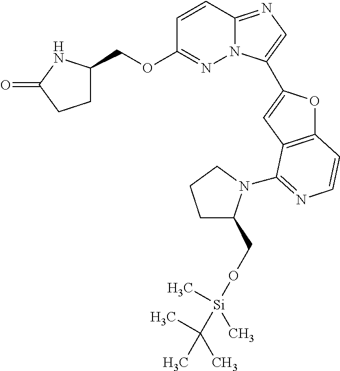 [SMILES: CC(C)(C)[Si](C)(C)OC[C@H]1CCCN1c1nccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (150 mg) in 1-propanol (13 mL) was added 2M potassium carbonate solution (0.72 mL), crude {4-[(2R)-2-({[tert-butyl(dimethyl)silyl]oxy}methyl)pyrrolidin-1-yl]furo[3,2-c]pyridin-2-yl}boronic acid (95% w/w; 444 mg), triphenylphosphine (12.6 mg) and PdCl₂(PPh₃)₂ (33.8 mg). The mixture was heated to reflux for 1 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 278 mg of the title compound.

¹H-NMR (300 MHz, CHLOROFORM-d), δ [ppm]=−0.05 (3H), 0.00 (3H), 0.84 (9H), 1.82-2.25 (5H), 2.39-2.52 (3H), 3.64-4.03 (4H), 4.20 (1H), 4.26-4.38 (1H), 4.52 (2H), 5.99 (1H), 6.76-6.86 (2H), 7.71 (1H), 7.94 (1H), 8.02 (1H), 8.12 (1H).

### Example III-067

### (5R)-5-{[(3-{4-[(2R)-2-(Hydroxymethyl)pyrrolidin-1-yl]furo[3,2-c]pyridin-2-yl}imidazo[1,2-b]pyridazin-6-yl)oxy]methyl}pyrrolidin-2-one

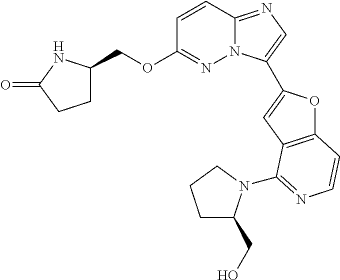 [SMILES: O=C1CC[C@H](COc2ccc3ncc(-c4cc5c(N6CCC[C@@H]6CO)nccc5o4)n3n2)N1]

To a stirred solution (5R)-5-{[(3-{4-[(2R)-2-({[tert-butyl(dimethyl)silyl]oxy}methyl)pyrrolidin-1-yl]furo[3,2-c]pyridin-2-yl}imidazo[1,2-b]pyridazin-6-yl)oxy]methyl}pyrrolidin-2-one (270 mg) in THF (25 mL) was added a solution of TBAF in THF (0.96 mL; c=1.0 mol/L). The mixture was stirred at room temperature for 4 h. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with methanol to give 71 mg of the title compound.

¹H-NMR (300 MHz, DMSO-d₆, detected signals), δ [ppm]=1.74-2.37 (7H), 3.32-3.40 (1H), 3.64 (2H), 3.85 (1H), 4.00 (1H), 4.28-4.40 (2H), 4.41-4.50 (1H), 5.03 (1H), 6.89 (1H), 6.97 (1H), 7.78 (1H), 7.91 (2H), 8.08 (1H), 8.14 (1H).

### Example III-068

### (5R)-5-[({3-[4-(Morpholin-4-yl)furo[3,2-c]pyridin-2-yl]imidazo[1,2-b]pyridazin-6-yl}oxy)methyl]pyrrolidin-2-one

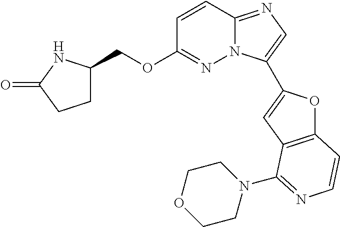 [SMILES: O=C1CC[C@H](COc2ccc3ncc(-c4cc5c(N6CCOCC6)nccc5o4)n3n2)N1]

To a stirred suspension of (5R)-5-(hydroxymethyl)pyrrolidin-2-one (97 mg) in anhydrous THF (12 mL) and anhydrous DMF (1.2 mL) was added sodium hydride (60% w/w in oil; 27 mg) at 0° C. and the mixture was stirred at room temperature for 30 minutes. 6-chloro-3-[4-(morpholin-4-yl)furo[3,2-c]pyridin-2-yl]imidazo-[1,2-b]pyridazine (200 mg) was added and the mixture was stirred at room temperature for 16 hours. A mixture of dichloromethane and methanol was added and the mixture was filtered through an aminophase silicagel column. The solvent was removed in vacuum. Silicagel chromatography gave a solid that was triturated with ethanol to give 150 mg of the title compound.

¹H-NMR (300 MHz, Pyr-d5, detected signals), δ [ppm]=1.88-2.03 (1H), 2.23-2.66 (3H), 3.84-4.02 (8H), 4.19-4.31 (1H), 4.35-4.45 (1H), 4.50-4.62 (1H), 6.80 (1H), 7.74 (1H), 8.10 (1H), 8.32 (1H), 8.48 (1H), 9.21 (1H).

### Example III-069

### (5R)-5-{[(3-{4-[Ethyl(2-methoxyethyl)amino]furo[3,2-c]pyridin-2-yl}imidazo[1,2-b]pyridazin-6-yl)oxy]methyl}pyrrolidin-2-one

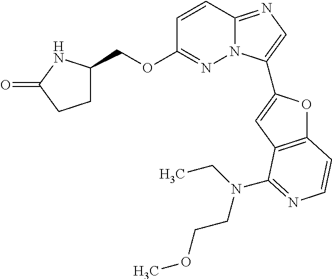 [SMILES: CCN(CCOC)c1nccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (750 mg) in 1-propanol (80 mL) was added 2M potassium carbonate solution (3.62 mL), crude {4-[Ethyl(2-methoxyethyl)amino]furo[3,2-c]pyridin-2-yl}boronic acid (80% w/w; 1.59 g), triphenylphosphine (63.2 mg) and PdCl₂(PPh₃)₂ (172 mg). The mixture was heated to reflux for 1 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with a mixture of dichloromethane and methanol to give 936 mg of the title compound.

¹H-NMR (300 MHz, Pyr-d5), δ [ppm]=1.39 (3H), 1.93-2.12 (1H), 2.23-2.39 (1H), 2.41-2.55 (1H), 2.56-2.72 (1H), 3.35 (3H), 3.71-3.81 (2H), 3.90 (2H), 3.97-4.08 (2H), 4.21-4.33 (1H), 4.37-4.48 (1H), 4.50-4.62 (1H), 6.77 (1H), 7.04 (1H), 7.72 (1H), 8.07 (1H), 8.26 (1H), 8.44 (1H), 9.17 (1H).

### Example III-070

### tert-Butyl 4-[2-(6-{[(2R)-5-oxopyrrolidin-2-yl]methoxy}imidazo[1,2-b]pyridazin-3-yl)furo[3,2-c]pyridin-4-yl]piperazine-1-carboxylate

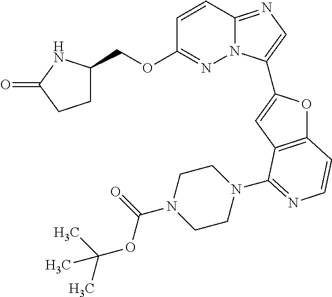 [SMILES: CC(C)(C)OC(=O)N1CCN(c2nccc3oc(-c4cnc5ccc(OC[C@H]6CCC(=O)N6)nn45)cc23)CC1]

To a stirred solution of (5R)-5-{[(3-bromoimidazo[1,2-b]pyridazin-6-yl)oxy]methyl}pyrrolidin-2-one (80 mg) in 1-propanol (8.5 mL) was added 2M potassium carbonate solution (0.39 mL), crude {4-[4-(tert-butoxycarbonyl)piperazin-1-yl]furo[3,2-c]pyridin-2-yl}boronic acid (80% w/w; 223 mg), triphenylphosphine (6.7 mg) and PdCl₂(PPh₃)₂ (18.4 mg). The mixture was heated to reflux for 2 h. The mixture was filtered through an aminophase silicagel column. The solvent was removed in vacuum. Silicagel chromatography gave a solid that was triturated with 2-propanol to give 100 mg of the title compound.

¹H-NMR (400 MHz, Pyr-d₅), δ [ppm]=1.51 (9H), 1.90-2.03 (1H), 2.26-2.38 (1H), 2.39-2.51 (1H), 2.55-2.67 (1H), 3.73 (4H), 3.81-3.91 (4H), 4.17-4.28 (1H), 4.37 (1H), 4.55 (1H), 6.74 (1H), 7.11 (1H), 7.68 (1H), 8.03 (1H), 8.23 (1H), 8.41 (1H), 9.11 (1H).

### Example III-071

### (5R)-5-[({3-[4-(Piperazin-1-yl)furo[3,2-c]pyridin-2-yl]imidazo[1,2-b]pyridazin-6-yl}oxy)methyl]pyrrolidin-2-one

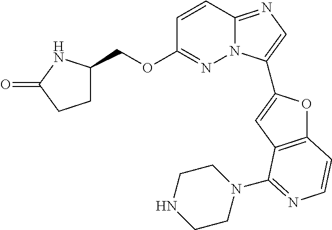 [SMILES: O=C1CC[C@H](COc2ccc3ncc(-c4cc5c(N6CCNCC6)nccc5o4)n3n2)N1]

To a stirred suspension of tert-butyl 4-[2-(6-{[(2R)-5-oxopyrrolidin-2-yl]methoxy}imidazo[1,2-b]pyridazin-3-yl)furo[3,2-c]pyridin-4-yl]piperazine-1-carboxylate (80 mg) in dichloromethane (5 mL) was added TFA (0.29 mL). The mixture was stirred at room temperature for 16 h. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was triturated with a mixture of 2-propanol and hexane to give 60 mg of the title compound.

¹H-NMR (400 MHz, DMSO-d₆), δ [ppm]=1.82-1.92 (1H), 2.09-2.37 (3H), 2.82-2.93 (4H), 3.59-3.67 (4H), 3.94-4.06 (1H), 4.34 (1H), 4.46 (1H), 7.01 (1H), 7.06 (1H), 7.65 (1H), 7.92 (1H), 7.99 (1H), 8.11-8.13 (1H), 8.16 (1H).

### Example III-072

### (5R)-5-{[(3-{4-[(2R,6S)-2,6-Dimethylmorpholin-4-yl]furo[3,2-c]pyridin-2-yl}imidazo[1,2-b]pyridazin-6-yl)oxy]methyl}pyrrolidin-2-one

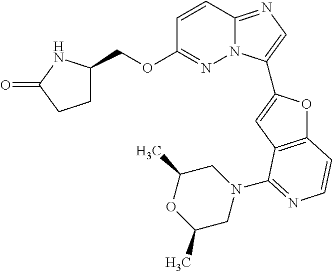 [SMILES: C[C@@H]1CN(c2nccc3oc(-c4cnc5ccc(OC[C@H]6CCC(=O)N6)nn45)cc23)C[C@H](C)O1]

To a stirred solution of (5R)-5-{[(3-bromoimidazo[1,2-b]pyridazin-6-yl)oxy]methyl}pyrrolidin-2-one (100 mg) in 1-propanol (9 mL) was added 2M potassium carbonate solution (0.48 mL), crude {4-[(2R,6S)-2,6-dimethylmorpholin-4-yl]furo[3,2-c]pyridin-2-yl}boronic acid (75% w/w; 236 mg), triphenylphosphine (8.4 mg) and PdCl₂(PPh₃)₂ (22.6 mg). The mixture was heated to reflux for 1 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave a solid that was triturated with a mixture of dichloromethane and hexane to give 77 mg of the title compound.

¹H-NMR (300 MHz, CHLOROFORM-d), δ [ppm]=1.29 (6H), 1.89-2.09 (1H), 2.38-2.54 (3H), 2.84 (2H), 3.75-3.93 (2H), 4.13-4.33 (4H), 4.52 (1H), 6.24 (1H), 6.81 (1H), 6.98 (1H), 7.43 (1H), 7.96 (1H), 8.07 (1H), 8.16 (1H).

### Example III-073

### (5R)-5-{[(3-{4-[(3-Hydroxypropyl)(methyl)amino]furo[3,2-c]pyridin-2-yl}imidazo[1,2-b]pyridazin-6-yl)oxy]methyl}pyrrolidin-2-one

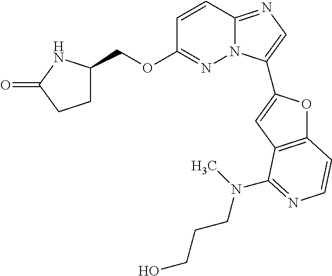 [SMILES: CN(CCCO)c1nccc2oc(-c3cnc4ccc(OC[C@H]5CCC(=O)N5)nn34)cc12]

To a stirred solution of (5R)-5-{[(3-{4-[(3-{[tert-butyl(dimethyl)silyl]oxy}propyl)(methyl)amino]furo[3,2-c]pyridin-2-yl}imidazo[1,2-b]pyridazin-6-yl)oxy]-methyl}pyrrolidin-2-one (133 mg) in THF (13 mL) was added a solution of TBAF in THF (0.48 mL; c=1.0 mol/L). The mixture was stirred at room temperature for 4 h. A half-saturated solution of sodium bicarbonate was added and the mixture was extracted with a mixture of ethyl acetate and methanol. The organic phase was dried (sodium sulfate) and the solvent was removed in vacuum. Silicagel chromatography gave a solid that was triturated with a mixture of dichloromethane and hexane to give 63 mg of the title compound.

¹H-NMR (300 MHz, DMSO-d₆), δ [ppm]=1.68-1.91 (3H), 2.06-2.35 (3H), 3.27 (3H), 3.46 (2H), 3.73 (2H), 4.00 (1H), 4.24-4.36 (1H), 4.45 (1H), 4.61 (1H), 6.90 (1H), 6.98 (1H), 7.74 (1H), 7.90-7.96 (2H), 8.07 (1H), 8.14 (1H).

### Example III-074

### (5R)-5-{[(3-{4-[4-(Dimethylamino)piperidin-1-yl]furo[3,2-c]pyridin-2-yl}imidazo[1,2-b]pyridazin-6-yl)oxy]methyl}pyrrolidin-2-one

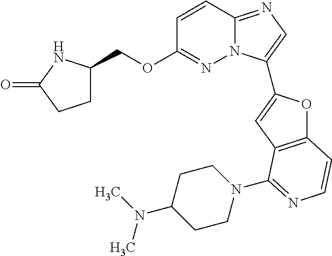 [SMILES: CN(C)C1CCN(c2nccc3oc(-c4cnc5ccc(OC[C@H]6CCC(=O)N6)nn45)cc23)CC1]

To a stirred solution of (5R)-5-{[(3-bromoimidazo[1,2-b]pyridazin-6-yl)oxy]methyl}pyrrolidin-2-one (60 mg) in 1-propanol (6.5 mL) was added 2M potassium carbonate solution (0.29 mL), crude {4-[4-(dimethylamino)piperidin-1-yl]furo-[3,2-c]pyridin-2-yl}boronic acid (80% w/w; 144 mg), triphenylphosphine (5.1 mg) and PdCl₂(PPh₃)₂ (13.8 mg). The mixture was heated to reflux for 4 h. The mixture was filtered through an aminophase silicagel column and the solvent was removed in vacuum. Aminophase silicagel chromatography gave a solid that was triturated with ethyl acetate to give 40 mg of the title compound.

¹H-NMR (300 MHz, DMSO-d₆), δ [ppm]=1.37-1.55 (2H), 1.77-1.91 (3H), 2.08-2.38 (10H), 2.99 (2H), 3.99 (1H), 4.27-4.48 (4H), 6.95-7.05 (2H), 7.60 (1H), 7.93-8.00 (2H), 8.10 (1H), 8.15 (1H).

### Example III-075

### (5R)-5-{[(3-{4-[(3S)-3-(Dimethylamino)pyrrolidin-1-yl]furo[3,2-c]pyridin-2-yl}imidazo[1,2-b]pyridazin-6-yl)oxy]methyl}pyrrolidin-2-one

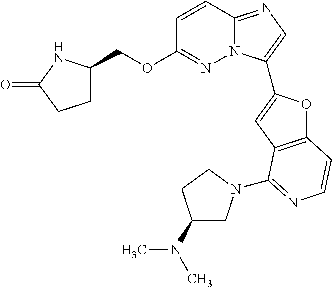 [SMILES: CN(C)[C@H]1CCN(c2nccc3oc(-c4cnc5ccc(OC[C@H]6CCC(=O)N6)nn45)cc23)C1]

To a stirred solution of (5R)-5-{[(3-bromoimidazo[1,2-b]pyridazin-6-yl)oxy]methyl}pyrrolidin-2-one (60 mg) in 1-propanol (6.5 mL) was added 2M potassium carbonate solution (0.29 mL), crude {4-[(3S)-3-(dimethylamino)pyrrolidin-1-yl]furo[3,2-c]pyridin-2-yl}boronic acid (80% w/w; 139 mg), triphenylphosphine (5.1 mg) and PdCl₂(PPh₃)₂ (13.8 mg). The mixture was heated to reflux for 4 h. The mixture was filtered through an aminophase silicagel column and the solvent was removed in vacuum. Aminophase silicagel chromatography gave a solid that was triturated with 2-propanol to give 40 mg of the title compound.

¹H-NMR (300 MHz, DMSO-d₆), δ [ppm]=1.71-1.92 (2H), 2.04-2.36 (10H), 2.66-2.81 (1H), 3.36 (1H), 3.53-3.70 (1H), 3.77-4.05 (3H), 4.16-4.42 (2H), 6.85 (1H), 6.92 (1H), 7.63 (1H), 7.89 (1H), 7.93 (1H), 8.03 (1H), 8.08 (1H).

### Example III-076

### (5R)-5-[({3-[4-(4-Phenylpiperazin-1-yl)furo[3,2-c]pyridin-2-yl]imidazo[1,2-b]pyridazin-6-yl}oxy)methyl]pyrrolidin-2-one

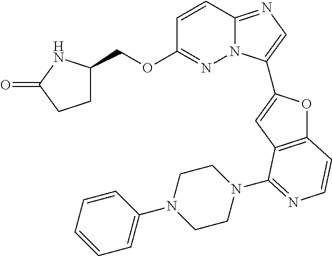 [SMILES: O=C1CC[C@H](COc2ccc3ncc(-c4cc5c(N6CCN(c7ccccc7)CC6)nccc5o4)n3n2)N1]

To 155 mg (0.5 mmol) (5R)-5-{[(3-bromoimidazo[1,2-b]pyridazin-6-yl)oxy]methyl}pyrrolidin-2-one in 6 mL propan-1-ol were added 242 mg (0.75 mmol) [4-(4-phenylpiperazin-1-yl)furo[3,2-c]pyridin-2-yl]boronic acid, 13 mg (50 μmol) triphenylphosphin, 29 mg (50 μmol) Pd(dba)₂ and 0.75 mL of a 2 M aqueous solution of sodium carbonate. The mixture was stirred at 110° C. for 2 h.

Water was added. The mixture was extracted with dichloromethane. The organic layer was dried over sodium sulfate and evaporated. The obtained crude product was digested in a mixture of DMSO and methanol to give 91 mg of the title compound.

¹H-NMR (400 MHz, DMSO-d6), δ [ppm]=1.83-1.96 (1H), 2.08-2.35 (3H), 3.30-3.36 (4H), 3.84 (4H), 4.02 (1H), 4.34-4.44 (1H), 4.46-4.55 (1H), 6.78 (1H), 6.94-7.05 (3H), 7.11 (1H), 7.17-7.27 (2H), 7.71 (1H), 7.95 (1H), 8.03 (1H), 8.12-8.21 (2H).

LC-MS (Method 13): Rₜ=0.88 min; MS (ESIpos) m/z=510 [M+H]⁺.

### Example III-077

### (5R)-5-{[(3-{4-[4-(Hydroxymethyl)piperidin-1-yl]furo[3,2-c]pyridin-2-yl}imidazo[1,2-b]pyridazin-6-yl)oxy]methyl}pyrrolidin-2-one

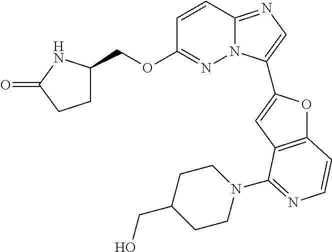 [SMILES: O=C1CC[C@H](COc2ccc3ncc(-c4cc5c(N6CCC(CO)CC6)nccc5o4)n3n2)N1]

To a stirred solution of (5R)-5-{[(3-{4-[4-({[tert-butyl(dimethyl)silyl]oxy}methyl)piperidin-1-yl]furo[3,2-c]pyridin-2-yl}imidazo[1,2-b]pyridazin-6-yl)oxy]methyl}pyrrolidin-2-one (130 mg) in THF (10 mL) was added a solution of TBAF in THF (0.56 mL; c=1.0 mol/L). The mixture was stirred at room temperature for 48 h. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with ethyl acetate to give 40 mg of the title compound.

¹H-NMR (300 MHz, DMSO-d₆), δ [ppm]=1.15-1.34 (2H), 1.66 (1H), 1.76 (2H), 1.83-1.92 (1H), 2.08-2.36 (3H), 2.97 (2H), 3.25-3.29 (2H), 3.99 (1H), 4.26-4.45 (4H), 4.48 (1H), 6.95-7.03 (2H), 7.60 (1H), 7.92-7.99 (2H), 8.10 (1H), 8.15 (1H).

### Example III-078

### (5R)-5-{[(3-{4-[(2R)-2-(Methoxymethyl)pyrrolidin-1-yl]furo[3,2-c]pyridin-2-yl}imidazo[1,2-b]pyridazin-6-yl)oxy]methyl}pyrrolidin-2-one

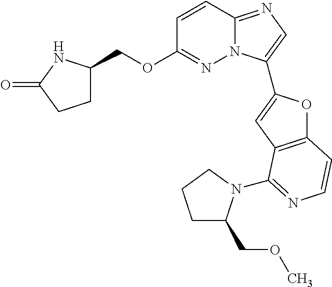 [SMILES: COC[C@H]1CCCN1c1nccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (80 mg) in 1-propanol (8.5 mL) was added 2M potassium carbonate solution (0.39 mL), crude {4-[(2R)-2-(methoxymethyl)pyrrolidin-1-yl]furo[3,2-c]pyridin-2-yl}boronic acid (80% w/w; 133 mg), triphenylphosphine (6.7 mg) and PdCl₂(PPh₃)₂ (18.4 mg). The mixture was heated to reflux for 2 h. The mixture was filtered and the solvent was removed in vacuum. Silicagel chromatography followed by aminophase silicagel chromatography gave a solid that was triturated with warm ethanol to give 80 mg of the title compound.

¹H-NMR (400 MHz, DMSO-d₆), δ [ppm]=1.78-2.34 (8H), 3.22-3.26 (3H), 3.28-3.32 (1H), 3.51-3.69 (2H), 3.88 (1H), 3.94-4.06 (1H), 4.28-4.43 (2H), 4.44-4.55 (1H), 6.89 (1H), 6.95 (1H), 7.75 (1H), 7.89-7.96 (2H), 8.07 (1H), 8.12 (1H).

### Example III-079

### (5R)-5-{[(3-{4-[(3R)-3-Methylmorpholin-4-yl]furo[3,2-c]pyridin-2-yl}imidazo[1,2-b]pyridazin-6-yl)oxy]methyl}pyrrolidin-2-one

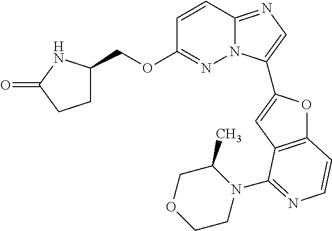 [SMILES: C[C@@H]1COCCN1c1nccc2oc(-c3cnc4ccc(OC[C@H]5CCC(=O)N5)nn34)cc12]

To 400 mg (1.3 mmol) (5R)-5-{[(3-bromoimidazo[1,2-b]pyridazin-6-yl)oxy]methyl}pyrrolidin-2-one in 21 mL propan-1-ol were added 1.25 g (2.6 mmol) {4-[(3R)-3-methylmorpholin-4-yl]furo[3,2-c]pyridin-2-yl}boronic acid, 59 mg (51 μmol) tetrakis(triphenylphosphin)-palladium(0), and 2 mL of a 2 M aqueous solution of sodium carbonate. The mixture was stirred at 110° C. for 17 h.

Water was added. The mixture was extracted with dichloromethane. The organic layer was dried over sodium sulfate and evaporated. The obtained crude product was digested in methanol to give 440 mg of the title compound.

¹H-NMR (400 MHz, DMSO-d6), δ [ppm]=1.27 (3H), 1.83-1.96 (1H), 2.09-2.40 (4H), 3.36-3.43 (1H), 3.52-3.64 (1H), 3.73 (2H), 3.90-4.07 (2H), 4.15 (1H), 4.30 (1H), 4.51 (2H), 7.00-7.10 (2H), 7.68 (1H), 7.95 (1H), 8.02 (1H), 8.14 (1H), 8.19 (1H).

LC-MS (Method 14): Rₜ=0.69 min; MS (ESIpos) m/z=449 [M+H]⁺.

### Example III-080

### (5R)-5-{[(3-{4-[(2-tert-Butoxyethyl)(ethyl)amino]furo[3,2-c]pyridin-2-yl}imidazo[1,2-b]pyridazin-6-yl)oxy]methyl}pyrrolidin-2-one

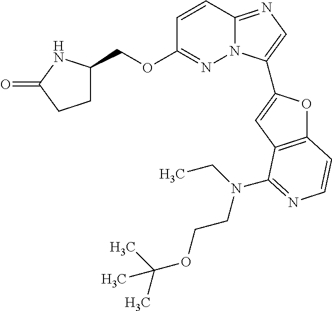 [SMILES: CCN(CCOC(C)(C)C)c1nccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (80 mg) in 1-propanol (8.5 mL) was added 2M potassium carbonate solution (0.39 mL), crude {4-[(2-tert-butoxyethyl)(ethyl)amino] furo-[3,2-c]pyridin-2-yl}boronic acid (50% w/w; 315 mg), triphenylphosphine (6.7 mg) and PdCl₂(PPh₃)₂ (18.4 mg). The mixture was heated to reflux for 2 h. The mixture was filtered and the solvent was removed in vacuum. Silicagel chromatography followed by aminophase silicagel chromatography gave a solid that was triturated with a mixture of ethyl acetate and hexane to give 60 mg of the title compound.

¹H-NMR (400 MHz, DMSO-d₆), δ [ppm]=1.06 (9H), 1.21-1.30 (3H), 1.80-1.92 (1H), 2.09-2.34 (3H), 3.49-3.60 (2H), 3.67-3.83 (4H), 3.91-4.06 (1H), 4.32 (1H), 4.46 (1H), 6.89 (1H), 7.00 (1H), 7.66 (1H), 7.89-7.96 (2H), 8.09 (1H), 8.16 (1H).

### Example III-081

### (5R)-5-{[(3-{4-[(2-Methoxyethyl)(methyl)amino]furo[3,2-c]pyridin-2-yl}imidazo[1,2-b]pyridazin-6-yl)oxy]methyl}pyrrolidin-2-one

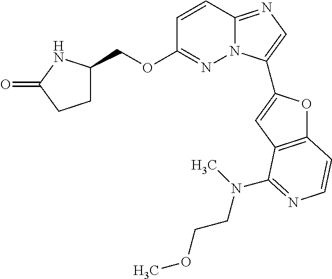 [SMILES: COCCN(C)c1nccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (100 mg) in 1-propanol (8.7 mL) was added 2M potassium carbonate solution (0.48 mL), crude {4-[(2-methoxyethyl)(methyl)amino]furo-[3,2-c]pyridin-2-yl}boronic acid (90% w/w; 179 mg), triphenylphosphine (8.4 mg) and PdCl₂(PPh₃)₂ (22.6 mg). The mixture was heated to reflux for 1 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Silicagel chromatography gave 74 mg of the title compound.

¹H-NMR (400 MHz, DMSO-d₆), δ [ppm]=1.79-1.91 (1H), 2.09-2.34 (3H), 3.25 (3H), 3.32 (3H), 3.54-3.63 (2H), 3.81-3.91 (2H), 3.96-4.05 (1H), 4.32 (1H), 4.43 (1H), 6.91 (1H), 6.99 (1H), 7.75 (1H), 7.90-7.96 (2H), 8.08 (1H), 8.14 (1H).

### Example III-082

### (5R)-5-{[(3-{4-[(2-Methoxyethyl)(propyl)amino]furo[3,2-c]pyridin-2-yl}imidazo[1,2-b]pyridazin-6-yl)oxy]methyl}pyrrolidin-2-one

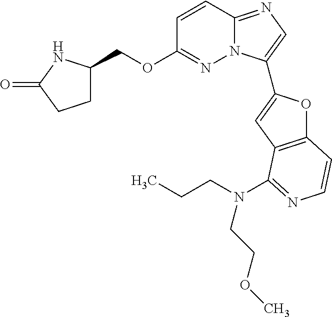 [SMILES: CCCN(CCOC)c1nccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (85 mg) in 1-propanol (5 mL) was added 2M potassium carbonate solution (0.41 mL), crude {4-[(2-methoxyethyl)(propyl)amino]furo-[3,2-c]pyridin-2-yl}boronic acid (70% w/w; 217 mg), triphenylphosphine (7.2 mg) and PdCl₂(PPh₃)₂ (19.2 mg). The mixture was heated to reflux for 2 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography followed by silicagel chromatography gave 40 mg of the title compound.

¹H-NMR (400 MHz, DMSO-d₆), δ [ppm]=0.90 (3H), 1.65 (2H), 1.84-1.95 (1H), 2.11-2.33 (3H), 3.25 (3H), 3.55-3.61 (2H), 3.62-3.71 (2H), 3.78-3.91 (2H), 3.99 (1H), 4.34 (1H), 4.44 (1H), 6.88-6.94 (1H), 7.01 (1H), 7.59 (1H), 7.91 (1H), 7.94 (1H), 8.10 (1H), 8.16 (1H).

### Example III-083

### (5R)-5-{[(3-{4-[(2S)-2-(Methoxymethyl)pyrrolidin-1-yl]furo[3,2-c]pyridin-2-yl}imidazo[1,2-b]pyridazin-6-yl)oxy]methyl}pyrrolidin-2-one

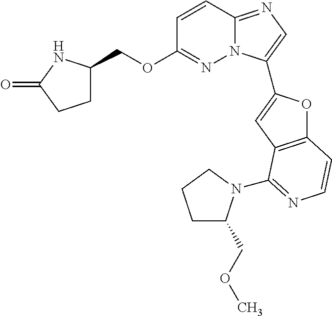 [SMILES: COC[C@@H]1CCCN1c1nccc2oc(-c3cnc4ccc(OC[C@H]5CCC(=O)N5)nn34)cc12]

To a stirred solution of (5R)-5-{[(3-bromoimidazo[1,2-b]pyridazin-6-yl)oxy]methyl}pyrrolidin-2-one (102 mg) in 1-propanol (5 mL) was added 2M potassium carbonate solution (0.49 mL), crude {4-[(2S)-2-(methoxymethyl)pyrrolidin-1-yl]furo[3,2-c]pyridin-2-yl}boronic acid (82% w/w; 221 mg), triphenylphosphine (8.6 mg) and PdCl₂(PPh₃)₂ (23.0 mg). The mixture was heated to reflux for 2 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with hexane to give 25 mg of the title compound.

¹H-NMR (400 MHz, DMSO-d₆, detected signals), δ [ppm]=1.75-1.89 (1H), 1.90-2.09 (4H), 2.10-2.35 (3H), 3.23 (3H), 3.57 (1H), 3.62-3.74 (1H), 3.82-3.92 (1H), 4.02 (1H), 4.31 (1H), 4.41-4.55 (2H), 6.90 (1H), 6.98 (1H), 7.78 (1H), 7.90-7.97 (2H), 8.08 (1H), 8.14 (1H).

### Example III-084

### (5R)-5-{[(3-{4-[(3S)-3-Methylmorpholin-4-yl]furo[3,2-c]pyridin-2-yl}imidazo[1,2-b]pyridazin-6-yl)oxy]methyl}pyrrolidin-2-one

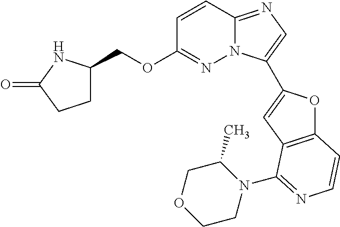 [SMILES: C[C@H]1COCCN1c1nccc2oc(-c3cnc4ccc(OC[C@H]5CCC(=O)N5)nn34)cc12]

To 115 mg (0.37 mmol) (5R)-5-{[(3-bromoimidazo[1,2-b]pyridazin-6-yl)oxy]methyl}pyrrolidin-2-one in 6 mL propan-1-ol were added 323 g (0.74 mmol) {4-[(3S)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Half-saturated brine was added. The mixture was extracted with dichloromethane. The organic layer was dried over sodium sulfate and evaporated. The obtained crude product was digested in methanol to give 127 mg of the title compound.

¹H-NMR (400 MHz, DMSO-d₆), δ [ppm]=1.27 (3H), 1.83-1.95 (1H), 2.12-2.38 (3H), 3.39 (1H), 3.58 (1H), 3.74 (2H), 3.92-4.06 (2H), 4.17 (1H), 4.38-4.46 (2H), 4.51-4.58 (1H), 7.02-7.09 (2H), 7.71 (1H), 7.92 (1H), 8.03 (1H), 8.14 (1H), 8.19 (1H).

LC-MS (Method 13): Rₜ=0.71 min; MS (ESIpos) m/z=449 [M+H]⁺.

### Example III-085

### (5S)-5-{[(3-{4-[(3R)-3-Methylmorpholin-4-yl]furo[3,2-c]pyridin-2-yl}imidazo[1,2-b]pyridazin-6-yl)oxy]methyl}pyrrolidin-2-one

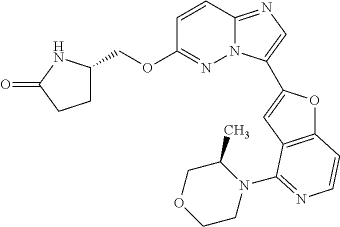 [SMILES: C[C@@H]1COCCN1c1nccc2oc(-c3cnc4ccc(OC[C@@H]5CCC(=O)N5)nn34)cc12]

To 115 mg (0.37 mmol) (5S)-5-{[(3-bromoimidazo[1,2-b]pyridazin-6-yl)oxy]methyl}pyrrolidin-2-one in 6 mL propan-1-ol were added 323 g (0.74 mmol) {4-[(3R)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Half-saturated brine was added. The mixture was extracted with dichloromethane. The organic layer was dried over sodium sulfate and evaporated. The obtained crude product was digested in methanol to give 88 mg of the title compound.

¹H-NMR (400 MHz, DMSO-d₆), δ [ppm]=1.24 (3H), 1.81-1.94 (1H), 2.09-2.38 (3H), 3.34-3.41 (1H), 3.49-3.61 (1H), 3.71 (2H), 3.88-4.04 (2H), 4.14 (1H), 4.38 (2H), 4.52 (1H), 6.98-7.07 (2H), 7.67 (1H), 7.92 (1H), 8.00 (1H), 8.12 (1H), 8.16 (1H).

LC-MS (Method 13): Rₜ=0.70 min; MS (ESIpos) m/z=449 [M+H]⁺.

### Example III-086

### (S)-5-{[(3-{4-[Ethyl(2-methoxyethyl)amino]furo[3,2-c]pyridin-2-yl}imidazo[1,2-b]pyridazin-6-yl)oxy]methyl}pyrrolidin-2-one

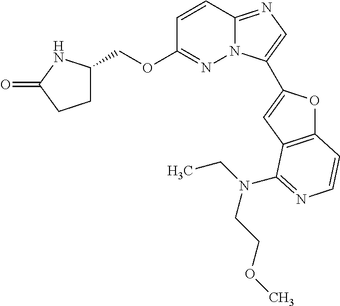 [SMILES: CCN(CCOC)c1nccc2oc(-c3cnc4ccc(OC[C@@H]5CCC(=O)N5)nn34)cc12]

To 130 mg (0.42 mmol) (5S)-5-{[(3-bromoimidazo[1,2-b]pyridazin-6-yl)oxy]methyl}pyrrolidin-2-one in 4.4 mL 1,4-doxane were added 356 mg (0.84 mmol) {4-[ethyl(2-methoxyethyl)amino]furo[3,2-c]pyridin-2-yl}boronic acid, 97 mg (84 μmol) tetrakis(triphenylphosphin)palladium(0) and 0.63 mL of a 2 M aqueous solution of sodium carbonate. The mixture was stirred at 100° C. for 24 h.

Saturated aqueous ammonium chloride solution was added. The mixture was extracted with ethyl acetate. The organic layer was dried and evaporated. The crude product was purified by HPLC to give 43 mg of the title compound.

¹H-NMR (400 MHz, DMSO-d₆), δ [ppm]=1.27 (3H), 1.86-1.99 (1H), 2.13-2.37 (3H), 3.29 (3H), 3.58-3.66 (2H), 3.75-3.91 (4H), 3.99-4.07 (1H), 4.38 (1H), 4.47 (1H), 6.95 (1H), 7.05 (1H), 7.68 (1H), 7.92-8.00 (2H), 8.13 (1H), 8.20 (1H), 8.31 (1H).

### Example III-087

### (5S)-5-{[(3-{4-[(3S)-3-Methylmorpholin-4-yl]furo[3,2-c]pyridin-2-yl}imidazo-[1,2-b]pyridazin-6-yl)oxy]methyl}pyrrolidin-2-one

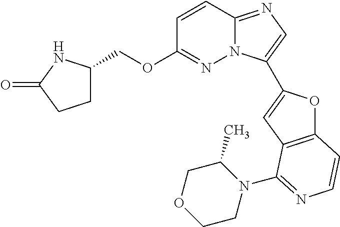 [SMILES: C[C@H]1COCCN1c1nccc2oc(-c3cnc4ccc(OC[C@@H]5CCC(=O)N5)nn34)cc12]

To 115 mg (0.37 mmol) (5S)-5-{[(3-bromoimidazo[1,2-b]pyridazin-6-yl)oxy]methyl}pyrrolidin-2-one in 6 mL propan-1-ol were added 323 g (0.74 mmol) {4-[(3S)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Half-saturated brine was added. The mixture was extracted with dichloromethane. The organic layer was dried over sodium sulfate and evaporated. The obtained crude product was digested in methanol to give 81 mg of the title compound.

¹H-NMR (400 MHz, DMSO-d₆), δ [ppm]=1.28 (3H), 1.84-1.95 (1H), 2.11-2.39 (3H), 3.39 (1H), 3.59 (1H), 3.73 (2H), 3.91-4.07 (2H), 4.15 (1H), 4.32 (1H), 4.49-4.57 (2H), 7.04 (1H), 7.07 (1H), 7.70 (1H), 7.92 (1H), 8.03 (1H), 8.14 (1H), 8.19 (1H).

LC-MS (Method 13): Rₜ=0.71 min; MS (ESIpos) m/z=449 [M+H]⁺.

### Example III-088

### (5S)-5-{[(3-{4-[(2S)-2-Methylmorpholin-4-yl]furo[3,2-c]pyridin-2-yl}imidazo-[1,2-b]pyridazin-6-yl)oxy]methyl}pyrrolidin-2-one

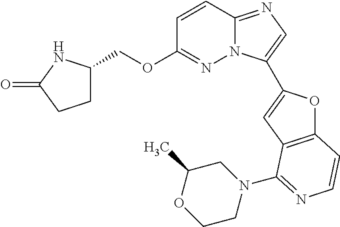 [SMILES: C[C@H]1CN(c2nccc3oc(-c4cnc5ccc(OC[C@@H]6CCC(=O)N6)nn45)cc23)CCO1]

To 115 mg (0.37 mmol) (5S)-5-{[(3-bromoimidazo[1,2-b]pyridazin-6-yl)oxy]methyl}pyrrolidin-2-one in 6 mL propan-1-ol were added 323 g (0.74 mmol) {4-[(2S)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 18 h.

Water was added. The mixture was extracted with dichloromethane. The organic layer was dried over sodium sulfate and evaporated. The obtained crude product was purified by flash chromatography to give 56 mg of the title compound.

¹H-NMR (400 MHz, DMSO-d₆), δ [ppm]=1.28 (3H), 1.84-1.95 (1H), 2.11-2.39 (3H), 3.39 (1H), 3.59 (1H), 3.73 (2H), 3.91-4.07 (2H), 4.15 (1H), 4.32 (1H), 4.49-4.57 (2H), 7.04 (1H), 7.07 (1H), 7.70 (1H), 7.92 (1H), 8.03 (1H), 8.14 (1H), 8.19 (1H).

LC-MS (Method 12): Rₜ=0.71 min; MS (ESIpos) m/z=449 [M+H]⁺.

### Example III-089

### (6R)-6-[({3-[4-(Morpholin-4-yl)furo[3,2-c]pyridin-2-yl]imidazo[1,2-b]pyridazin-6-yl}oxy)methyl]piperazin-2-one

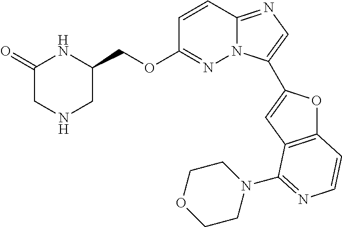 [SMILES: O=C1CNC[C@H](COc2ccc3ncc(-c4cc5c(N6CCOCC6)nccc5o4)n3n2)N1]

Step 1: In an ice bath 35 mg (0.87 mmol) sodium hydride (60% dispersion in mineral oil) were dispensed in 4 mL of anhydrous tetrahydrofurane. 200 mg (0.87 mmol) (6R)-4-(2,2-dimethylpropanoyl)-6-(hydroxymethyl)piperazin-2-one were slowly added. After complete addition, stirring at 0° C. was continued for 15 min. 155 mg (0.43 mmol) of 6-chloro-3-[4-(morpholin-4-yl)furo[3,2-c]pyridin-2-yl]imidazo[1,2-b]-pyridazine were added, the ice bath removed and the resulting mixture was stirred for 24 h at 40° C.

Brine was added. The mixture was extracted with ethyl acetate. The organic layer was dried over sodium sulfate and evaporated to give a crude product which was used in step 2.

Step 2: 6 mL dichlormethane were added to the crude material from step 1. 3.3 mL TFA were added and the mixture was stirred at room temperature for 24 h.

1 N aqueous ammonia was added until basic pH was reached. The mixture was extracted with dichloromethane. The organic layer was dried over sodium sulfate and evaporated. The crude product was purified by HPLC to give 19 mg of the title compound.

¹H-NMR (400 MHz, DMSO-d₆), δ [ppm]=2.91 (1H), 3.01-3.08 (1H), 3.21 (2H), 3.65-3.85 (11H), 4.43 (1H), 4.56 (1H), 7.08 (1H), 7.15 (1H), 7.75 (1H), 7.84 (1H), 8.06 (1H), 8.17 (1H), 8.21 (1H).

LC-MS (Method 12): Rₜ=0.53 min; MS (ESIpos) m/z=450 [M+H]⁺.

### Example III-090

### (6R)-6-{[(3-{4-[Ethyl(2-methoxyethyl)amino]furo[3,2-c]pyridin-2-yl}imidazo[1,2-b]pyridazin-6-yl)oxy]methyl}piperazin-2-one

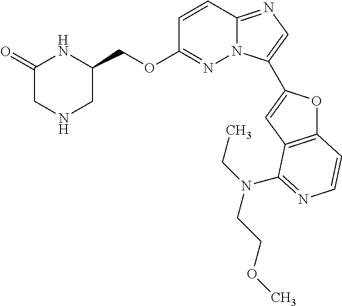 [SMILES: CCN(CCOC)c1nccc2oc(-c3cnc4ccc(OC[C@H]5CNCC(=O)N5)nn34)cc12]

Step 1: In an ice bath 52 mg (1.3 mmol) sodium hydride (60% dispersion in mineral oil) were dispensed in 6 mL of anhydrous tetrahydrofurane. 300 mg (1.3 mmol) (6R)-4-(2,2-dimethylpropanoyl)-6-(hydroxymethyl)piperazin-2-one were slowly added. After complete addition, stirring at 0° C. was continued for 15 min. 363 mg (0.98 mmol) of 2-(6-chloroimidazo[1,2-b]pyridazin-3-yl)-N-ethyl-N-(2-methoxy-ethyl)furo[3,2-c]pyridin-4-amine were added, the ice bath removed and the resulting mixture was stirred for 24 h at 40° C.

Brine was added. The mixture was extracted with ethyl acetate. The organic layer was dried over sodium sulfate and evaporated to give a crude product which was used in step 2.

Step 2: 10 mL dichlormethane were added to the crude material from step 1. 7.5 mL TFA were added and the mixture was stirred at room temperature for 24 h.

1 N aqueous ammonia was added until basic pH was reached. The mixture was extracted with dichloromethane. The organic layer was dried over sodium sulfate and evaporated. The crude product was purified by HPLC to give 86 mg of the title compound.

¹H-NMR (400 MHz, DMSO-d6), δ [ppm]=1.27 (3H) 2.92 (1H) 3.01 (1H) 3.21 (2H) 3.62 (2H) 3.72-3.90 (5H) 4.38 (1H) 4.58 (1H) 6.94 (1H) 7.07 (1H) 7.70 (1H) 7.85-7.88 (1H) 7.97 (1H) 8.13 (1H) 8.20 (1H).

LC-MS (Method 12): Rₜ=0.52 min; MS (ESIpos) m/z=466 [M+H]⁺.

### Example III-091

### 6-Methoxy-3-[4-(4-phenylpiperazin-1-yl)furo[3,2-c]pyridin-2-yl]imidazo[1,2-b]pyridazine

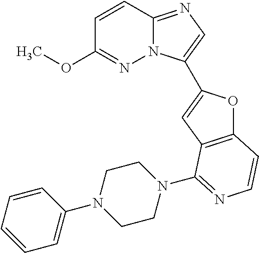 [SMILES: COc1ccc2ncc(-c3cc4c(N5CCN(c6ccccc6)CC5)nccc4o3)n2n1]

¹H-NMR (400 MHz, DMSO-d₆), δ [ppm]=3.29-3.35 (4H), 3.80-3.88 (4H), 4.14 (3H), 6.78 (1H), 6.97 (2H), 7.04 (1H), 7.12 (1H), 7.19-7.26 (2H), 7.48-7.63 (1H), 7.78 (1H), 8.03 (1H), 8.13 (1H), 8.16 (1H)

LC-MS (Method 13): Rₜ=1.04 min; MS (ESIpos) m/z=427 [M+H]⁺.

### Example III-092

### 6-Methoxy-3-{4-[(3R)-3-methylmorpholin-4-yl]furo[3,2-c]pyridin-2-yl}imidazo[1,2-b]pyridazine

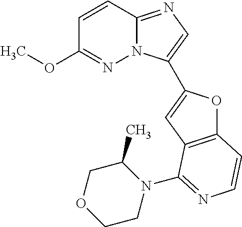 [SMILES: COc1ccc2ncc(-c3cc4c(N5CCOC[C@H]5C)nccc4o3)n2n1]

To 84 mg (0.37 mmol) (3-bromo-6-methoxyimidazo[1,2-b]pyridazine in 6 mL propan-1-ol were added 323 mg (0.74 mmol) {4-[(3R)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Water was added. The mixture was extracted with dichloromethane. The organic layer was dried over sodium sulfate and evaporated. The obtained crude product was digested in methanol to give 69 mg of the title compound.

¹H-NMR (400 MHz, DMSO-d₆), δ [ppm]=1.26 (3H), 3.36 (1H), 3.55 (1H), 3.69 (2H), 3.92 (1H), 4.08 (3H), 4.15 (1H), 4.49 (1H), 6.98-7.05 (2H), 7.71 (1H), 7.99 (1H), 8.09 (1H), 8.13 (1H).

LC-MS (Method 13): Rₜ=0.78 min; MS (ESIpos) m/z=466 [M+H]⁺.

### Example III-093

### 6-Methoxy-3-{4-[(3S)-3-methylmorpholin-4-yl]furo[3,2-c]pyridin-2-yl}imidazo[1,2-b]pyridazine

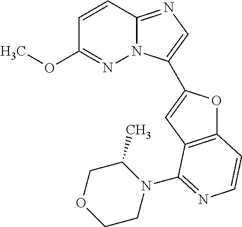 [SMILES: COc1ccc2ncc(-c3cc4c(N5CCOC[C@@H]5C)nccc4o3)n2n1]

To 84 mg (0.37 mmol) (3-bromo-6-methoxyimidazo[1,2-b]pyridazine in 6 mL propan-1-ol were added 323 mg (0.74 mmol) {4-[(3S)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Water was added. The mixture was extracted with dichloromethane. The organic layer was dried over sodium sulfate and evaporated. The obtained crude product was digested in methanol to give 84 mg of the title compound.

¹H-NMR (400 MHz, DMSO-d₆), δ [ppm]=1.29 (3H), 3.39 (1H), 3.57 (1H), 3.72 (2H), 3.94 (1H), 4.11 (3H), 4.18 (1H), 4.52 (1H), 7.02-7.08 (2H), 7.75 (1H), 8.02 (1H), 8.12 (1H), 8.16 (1H).

LC-MS (Method 17): Rₜ=0.76 min; MS (ESIpos) m/z=366 [M+H]⁺.

### Example III-094

### 6-Methoxy-3-{4-[(2S)-2-methylmorpholin-4-yl]furo[3,2-c]pyridin-2-yl}imidazo[1,2-b]pyridazine

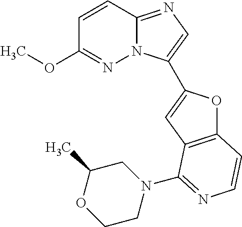 [SMILES: COc1ccc2ncc(-c3cc4c(N5CCO[C@@H](C)C5)nccc4o3)n2n1]

To 84 mg (0.37 mmol) (3-bromo-6-methoxyimidazo[1,2-b]pyridazine in 6 mL propan-1-ol were added 312 mg (0.74 mmol) {4-[(2S)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Water was added. The mixture was extracted with dichloromethane. The organic layer was dried over sodium sulfate and evaporated. The obtained crude product was digested in a mixture of DMSO and methanol to give 88 mg of the title compound.

¹H-NMR (400 MHz, DMSO-d₆), δ [ppm]=1.15 (3H), 2.82 (1H), 3.01-3.15 (1H), 3.59-3.76 (2H), 3.94 (1H), 4.12 (3H), 4.23 (2H), 7.05 (1H), 7.12 (1H), 7.77 (1H), 8.03 (1H), 8.12-8.21 (2H).

LC-MS (Method 17): Rₜ=0.79 min; MS (ESIpos) m/z=366 [M+H]⁺.

### Example III-095

### trans-3-({3-[4-(Morpholin-4-yl)furo[3,2-c]pyridin-2-yl]imidazo[1,2-b]pyridazin-6-yl}oxy)cyclobutanamine

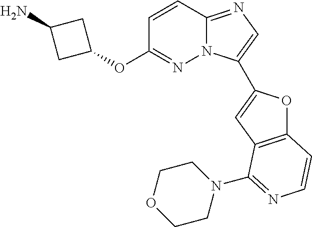 [SMILES: N[C@H]1C[C@H](Oc2ccc3ncc(-c4cc5c(N6CCOCC6)nccc5o4)n3n2)C1]

To a stirred suspension of tert-butyl [trans-3-({3-[4-(morpholin-4-yl)furo[3,2-c]pyridin-2-yl]imidazo[1,2-b]pyridazin-6-yl}oxy)cyclobutyl]carbamate (59 mg) in dichloromethane (2 mL) was added TFA (1.1 mL). The mixture was stirred at room temperature for 2 h. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was triturated with hexane to give 27 mg of the title compound.

¹H-NMR (400 MHz, DMSO-d₆), δ [ppm]=1.82-2.08 (2H), 2.20 (2H), 2.38-2.44 (2H), 3.59-3.72 (5H), 3.78-3.85 (4H), 5.32-5.42 (1H), 6.98 (1H), 7.09 (1H), 7.55 (1H), 8.01 (1H), 8.09 (1H), 8.11 (1H).

### Example III-096

### cis-3-({3-[4-(4-Methylpiperazin-1-yl)furo[3,2-c]pyridin-2-yl]imidazo[1,2-b]pyridazin-6-yl}oxy)cyclobutanamine

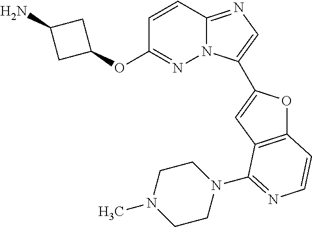 [SMILES: CN1CCN(c2nccc3oc(-c4cnc5ccc(O[C@H]6C[C@@H](N)C6)nn45)cc23)CC1]

To a stirred suspension of tert-butyl [cis-3-({3-[4-(4-methylpiperazin-1-yl)furo-[3,2-c]pyridin-2-yl]imidazo[1,2-b]pyridazin-6-yl}oxy)cyclobutyl]carbamate (180 mg) in dichloromethane (15 mL) was added HCl in dioxane (2.17 mL; c=4.0 M). The mixture was stirred at room temperature for 1 h. The solvent was removed in vacuum. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was dissolved in water and freeze dried to give 76 mg of the title compound.

¹H-NMR (300 MHz, DMSO-d₆, detected signals), δ [ppm]=2.17-2.30 (5H), 2.51 (2H), 3.25 (4H), 3.61-3.78 (5H), 5.28-5.44 (1H), 7.00 (1H), 7.06 (1H), 7.56 (1H), 7.99 (1H), 8.07-8.18 (2H).

### Example III-097

2-{6-[(trans-3-Aminocyclobutyl)oxy]imidazo[1,2-b]pyridazin-3-yl}-N-methyl-N-[3-(pyrrolidin-1-yl)propyl]furo[3,2-c]pyridin-4-amine

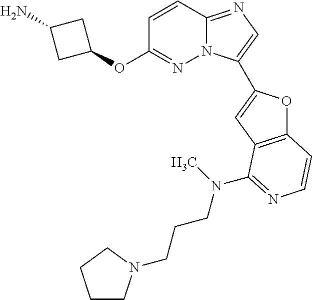 [SMILES: CN(CCCN1CCCC1)c1nccc2oc(-c3cnc4ccc(O[C@H]5C[C@H](N)C5)nn34)cc12]

To a stirred suspension of tert-butyl (trans-3-{[3-(4-{methyl[3-(pyrrolidin-1-yl)propyl]amino}furo[3,2-c]pyridin-2-yl)imidazo[1,2-b]pyridazin-6-yl]oxy}cyclo-butyl)carbamate (120 mg) in dichloromethane (1 mL) was added TFA (0.4 mL). The mixture was stirred at room temperature for 2 h. The solvent was removed in vacuum. A saturated solution of sodium bicarbonate was added and the mixture was extracted with ethyl acetate. The organic phase was dried (sodium sulfate) and the solvent was removed in vacuum. Silicagel chromatography gave a solid that was triturated with a mixture of ethyl acetate and hexane to give 83 mg of the title compound.

¹H-NMR (300 MHz, Pyr-d5), δ [ppm]=1.61 (4H), 1.93-2.09 (2H), 2.35-2.63 (10H), 3.58 (3H), 3.80-3.90 (1H), 4.02 (2H), 5.60 (1H), 6.84 (1H), 7.03 (1H), 7.91 (1H), 8.07 (1H), 8.28 (1H), 8.41 (1H).

### Example III-098

### (3R)-1-(2-{6-[(trans-3-Aminocyclobutyl)oxy]imidazo[1,2-b]pyridazin-3-yl}furo[3,2-c]pyridin-4-yl)-N,N-dimethylpyrrolidin-3-amine

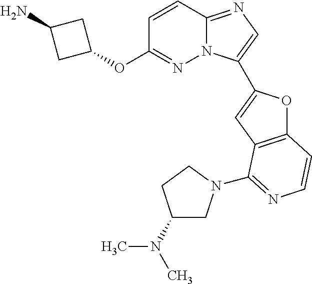 [SMILES: CN(C)[C@@H]1CCN(c2nccc3oc(-c4cnc5ccc(O[C@H]6C[C@H](N)C6)nn45)cc23)C1]

To a stirred suspension of tert-butyl {trans-3-[(3-{4-[(3R)-3-(dimethylamino)pyrrolidin-1-yl]furo[3,2-c]pyridin-2-yl}imidazo[1,2-b]pyridazin-6-yl)oxy]cyclobutyl}carbamate (133 mg) in dichloromethane (1 mL) was added TFA (0.48 mL). The mixture was stirred at room temperature for 2 h. The solvent was removed in vacuum. A saturated solution of sodium bicarbonate was added and the mixture was extracted with ethyl acetate. The organic phase was dried (sodium sulfate) and the solvent was removed in vacuum. Silicagel chromatography followed by aminophase silicagel chromatography gave 27 mg of the title compound.

¹H-NMR (300 MHz, DMSO-d₆), δ [ppm]=1.75-1.99 (3H), 2.22 (9H), 2.33-2.43 (2H), 2.74-2.89 (1H), 3.37-3.49 (1H), 3.54-3.68 (1H), 3.70-3.85 (1H), 3.87-4.02 (2H), 5.31-5.44 (1H), 6.87 (1H), 6.95 (1H), 7.63 (1H), 7.91 (1H), 8.04 (1H), 8.10 (1H).

### Example III-099

### trans-3-({3-[4-(4-tert-butylpiperazin-1-yl)furo[3,2-c]pyridin-2-yl]imidazo[1,2-b]pyridazin-6-yl}oxy)cyclobutanamine

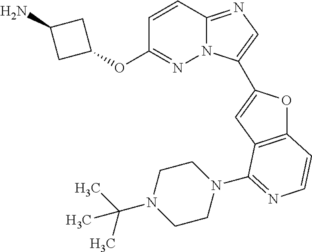 [SMILES: CC(C)(C)N1CCN(c2nccc3oc(-c4cnc5ccc(O[C@H]6C[C@H](N)C6)nn45)cc23)CC1]

To a stirred suspension of tert-butyl [trans-3-({3-[4-(4-tert-butylpiperazin-1-yl)furo[3,2-c]pyridin-2-yl]imidazo[1,2-b]pyridazin-6-yl}oxy)cyclobutyl]carbamate (160 mg) in dichloromethane (25 mL) was added TFA (0.55 mL). The mixture was stirred at room temperature for 16 h. The solvent was removed in vacuum. A saturated solution of sodium bicarbonate was added and the mixture was extracted with ethyl acetate. The organic phase was dried (sodium sulfate) and the solvent was removed in vacuum. Aminophase silicagel chromatography gave a solid that was triturated with ethyl acetate to give 50 mg of the title compound.

¹H-NMR (400 MHz, Pyr-d₅), δ [ppm]=1.05-1.12 (9H), 2.05 (2H), 2.37-2.48 (2H), 2.55-2.67 (2H), 2.80-2.92 (4H), 3.82-3.94 (1H), 4.02-4.11 (4H), 5.53 (1H), 6.83 (1H), 7.12 (1H), 7.81 (1H), 8.04 (1H), 8.30 (1H), 8.42 (1H).

### Example III-100

### 2-{6-[(trans-3-Aminocyclobutyl)oxy]imidazo[1,2-b]pyridazin-3-yl}-N-methyl-N-(1-methylpiperidin-4-yl)furo[3,2-c]pyridin-4-amine

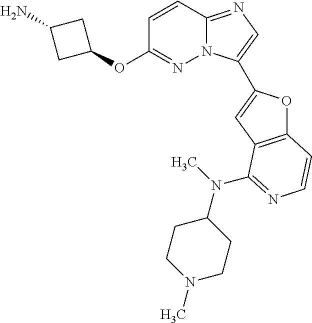 [SMILES: CN1CCC(N(C)c2nccc3oc(-c4cnc5ccc(O[C@H]6C[C@H](N)C6)nn45)cc23)CC1]

To a stirred suspension of tert-butyl {trans-3-[(3-{4-[methyl(1-methylpiperidin-4-yl)amino]furo[3,2-c]pyridin-2-yl}imidazo[1,2-b]pyridazin-6-yl)oxy]cyclobutyl}carbamate (68 mg) in dichloromethane (1 mL) was added TFA (0.24 mL). The mixture was stirred at room temperature for 24 h. The solvent was removed in vacuum. A saturated solution of sodium bicarbonate was added and the mixture was extracted with ethyl acetate. The organic phase was dried (sodium sulfate) and the solvent was removed in vacuum. Silicagel chromatography gave a solid that was triturated with a mixture of dichloromethane and hexane to give 40 mg of the title compound.

¹H-NMR (400 MHz, DMSO-d₆, detected signals), δ [ppm]=1.61 (2H), 1.84 (2H), 1.93-2.03 (2H), 2.13-2.25 (4H), 2.39 (2H), 2.85 (2H), 3.52-3.71 (1H), 4.59-4.75 (1H), 5.28-5.49 (1H), 6.88-6.93 (1H), 6.97 (1H), 7.71 (1H), 7.94 (1H), 8.06 (1H), 8.12 (1H).

### Example III-101

2-{6-[(trans-3-Aminocyclobutyl)oxy]imidazo[1,2-b]pyridazin-3-yl}-N-ethyl-N-(2-methoxyethyl)furo[3,2-c]pyridin-4-amine

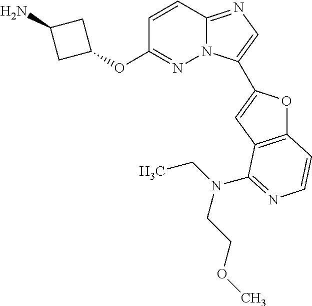 [SMILES: CCN(CCOC)c1nccc2oc(-c3cnc4ccc(O[C@H]5C[C@H](N)C5)nn34)cc12]

To a stirred suspension of tert-butyl {trans-3-[(3-{4-[ethyl(2-methoxyethyl)amino]furo[3,2-c]pyridin-2-yl}imidazo[1,2-b]pyridazin-6-yl)oxy]cyclobutyl}carbamate (80 mg) in dichloromethane (5 mL) was added TFA (0.30 mL). The mixture was stirred at room temperature for 16 h. The solvent was removed in vacuum. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was triturated with ether to give 50 mg of the title compound.

¹H-NMR (400 MHz, Pyr-d₅, detected signals), δ [ppm]=1.39 (3H), 2.41-2.60 (4H), 3.29-3.33 (3H), 3.76 (2H), 3.81-3.89 (1H), 3.93 (2H), 4.04 (2H), 5.48-5.59 (1H), 6.79 (1H), 6.97 (1H), 7.69 (1H), 8.00 (1H), 8.20 (1H), 8.36 (1H).

### Example III-102

### [(2R)-1-(2-{6-[(trans-3-Aminocyclobutyl)oxy]imidazo[1,2-b]pyridazin-3-yl}furo[3,2-c]pyridin-4-yl)pyrrolidin-2-yl]methanol

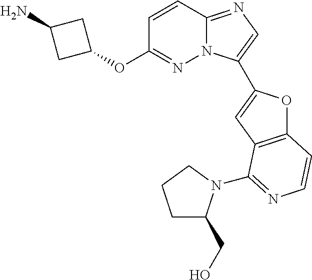 [SMILES: N[C@H]1C[C@H](Oc2ccc3ncc(-c4cc5c(N6CCC[C@@H]6CO)nccc5o4)n3n2)C1]

To a stirred suspension of tert-butyl {trans-3-[(3-{4-[(2R)-2-(hydroxymethyl)pyrrolidin-1-yl]furo[3,2-c]pyridin-2-yl}imidazo[1,2-b]pyridazin-6-yl)oxy]cyclobutyl}carbamate (132 mg) in dichloromethane (1 mL) was added TFA (0.39 mL). The mixture was stirred at room temperature for 4 h. The solvent was removed in vacuum. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was triturated with methanol to give 73 mg of the title compound.

¹H-NMR (400 MHz, DMSO-d₆, detected signals), δ [ppm]=1.84-2.11 (5H), 2.15-2.31 (2H), 2.37 (2H), 3.39 (1H), 3.54-3.75 (3H), 3.88 (1H), 4.38 (1H), 5.57 (1H), 6.88 (1H), 6.95 (1H), 7.67 (1H), 7.92 (1H), 8.05 (1H), 8.10 (1H).

### Example III-103

### trans-3-({3-[4-(Piperazin-1-yl)furo[3,2-c]pyridin-2-yl]imidazo[1,2-b]pyridazin-6-yl}oxy)cyclobutanamine

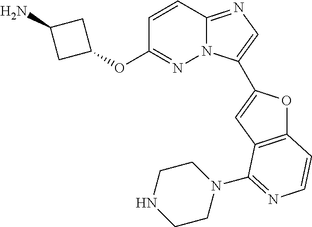 [SMILES: N[C@H]1C[C@H](Oc2ccc3ncc(-c4cc5c(N6CCNCC6)nccc5o4)n3n2)C1]

To a stirred suspension of tert-butyl 4-{2-[6-({trans-3-[(tert-butoxycarbonyl)-amino]cyclobutyl}oxy)imidazo[1,2-b]pyridazin-3-yl]furo[3,2-c]pyridin-4-yl}piperazine-1-carboxylate (80 mg) in dichloromethane (5 mL) was added TFA (0.25 mL). The mixture was stirred at room temperature for 16 h. The solvent was removed in vacuum. A saturated solution of potassium carbonate was added and the mixture was extracted with a mixture of dichloromethane and methanol. The organic phase was dried (sodium sulfate) and the solvent was removed in vacuum. Silicagel chromatography gave 40 mg of the title compound.

¹H-NMR (400 MHz, DMSO-d₆, detected signals), δ [ppm]=2.15-2.27 (2H), 2.86-2.92 (4H), 3.59-3.71 (5H), 5.34 (1H), 6.98 (1H), 7.01-7.06 (1H), 7.54 (1H), 7.98 (1H), 8.08 (1H), 8.12 (1H).

### Example III-104

### trans-3-[(3-{4-[(2R,6S)-2,6-Dimethylmorpholin-4-yl]furo[3,2-c]pyridin-2-yl}imidazo[1,2-b]pyridazin-6-yl)oxy]cyclobutanamine

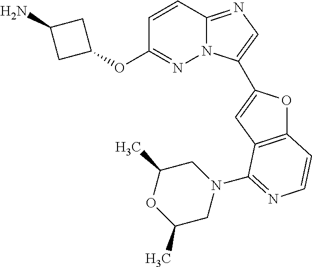 [SMILES: C[C@@H]1CN(c2nccc3oc(-c4cnc5ccc(O[C@H]6C[C@H](N)C6)nn45)cc23)C[C@H](C)O1]

To a stirred suspension of tert-butyl {trans-3-[(3-{4-[(2R,6S)-2,6-dimethylmorpholin-4-yl]furo[3,2-c]pyridin-2-yl}imidazo[1,2-b]pyridazin-6-yl)oxy]cyclobutyl}carbamate (170 mg) in dichloromethane (1 mL) was added TFA (0.49 mL). The mixture was stirred at room temperature for 4 h. The solvent was removed in vacuum. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was triturated with methanol to give 94 mg of the title compound.

¹H-NMR (300 MHz, CHLOROFORM-d, detected signals), δ [ppm]=1.32 (3H), 1.34 (3H), 2.21-2.38 (2H), 2.65 (2H), 2.80-2.95 (2H), 3.83-4.00 (3H), 4.25 (2H), 5.39 (1H), 6.80 (1H), 7.00 (1H), 7.52 (1H), 7.91 (1H), 8.08 (1H), 8.14 (1H).

### Example III-105

### 3-[(2-{6-[(trans-3-Aminocyclobutyl)oxy]imidazo[1,2-b]pyridazin-3-yl}furo-[3,2-c]pyridin-4-yl)(methyl)amino]propan-1-ol

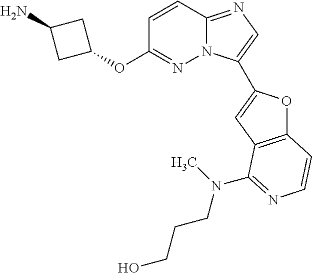 [SMILES: CN(CCCO)c1nccc2oc(-c3cnc4ccc(O[C@H]5C[C@H](N)C5)nn34)cc12]

To a stirred suspension of tert-butyl {trans-3-[(3-{4-[(3-hydroxypropyl)(methyl)-amino]furo[3,2-c]pyridin-2-yl}imidazo[1,2-b]pyridazin-6-yl)oxy]cyclobutyl}carbamate (40 mg) in dichloromethane (0.5 mL) was added TFA (0.12 mL). The mixture was stirred at room temperature for 4 h. The solvent was removed in vacuum. A saturated solution of potassium carbonate was added and the mixture was extracted with a mixture of dichloromethane and methanol. The organic phase was dried (sodium sulfate) and the solvent was removed in vacuum. Silicagel chromatography gave a solid that was triturated with methanol to give 30 mg of the title compound.

¹H-NMR (400 MHz, DMSO-d₆, detected signals), δ [ppm]=1.72-1.84 (2H), 2.22 (2H), 2.34-2.42 (2H), 3.34 (3H), 3.48 (2H), 3.61 (1H), 3.79 (2H), 5.40-5.52 (1H), 6.90 (1H), 6.97 (1H), 7.72 (1H), 7.93 (1H), 8.07 (1H), 8.12 (1H).

### Example III-106

### trans-3-({3-[4-(4-Phenyl piperazin-1-yl)furo[3,2-c]pyridin-2-yl]imidazo[1,2-b]pyridazin-6-yl}oxy)cyclobutanamine

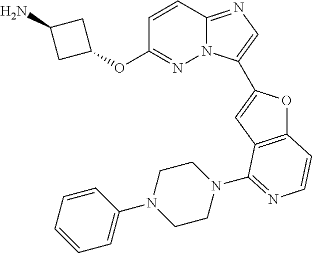 [SMILES: N[C@H]1C[C@H](Oc2ccc3ncc(-c4cc5c(N6CCN(c7ccccc7)CC6)nccc5o4)n3n2)C1]

Step 1: To 192 mg (0.5 mmol) tert-butyl {trans-3-[(3-bromoimidazo[1,2-b]pyridazin-6-yl)oxy]cyclobutyl}-carbamate in 6 mL propan-1-ol were added 242 mg (0.75 mmol) [4-(4-phenylpiperazin-1-yl)furo[3,2-c]pyridin-2-yl]boronic acid, 13 mg (50 μmol) triphenylphosphin, 29 mg (50 μmol) Pd(dba)₂ and 0.75 mL of a 2 M aqueous solution of potassium carbonate. The mixture was stirred at 110° C. for 2 h.

Water was added. The mixture was extracted with dichloromethane. The organic layer was separated, dried over sodium sulfate, and evaporated. The crude product (321 mg) was used without further purification in the subsequent step 2.

Step 2: 10 mL Dichlormethane were added to the crude material from step 1. 5 mL TFA were added and the mixture was stirred at room temperature for 15 min.

5 mL aqueous ammonia (25% in water) were added. Water was added. The mixture was extracted with dichloromethane. The organic layer was dried over sodium sulfate and evaporated. The crude product was purified by HPLC to give 32 mg of the title compound.

¹H-NMR (400 MHz, DMSO-d₆), δ [ppm]=2.32-2.41 (2H), 2.49-2.55 (3H), 3.35-3.40 (6H), 3.73 (2H), 3.87-3.94 (4H), 5.40-5.48 (1H), 6.77 (1H), 6.98-7.04 (3H), 7.09-7.13 (1H), 7.18-7.24 (2H), 7.61 (1H), 8.03 (1H), 8.11-8.17 (2H).

LC-MS (Method 13): Rₜ=0.73 min; MS (ESIpos) m/z=482 [M+H]⁺.

### Example III-107

### trans-3-[(3-{4-[(3R)-3-Methyl morpholin-4-yl]furo[3,2-c]pyridin-2-yl}imidazo[1,2-b]pyridazin-6-yl)oxy]cyclobutanamine

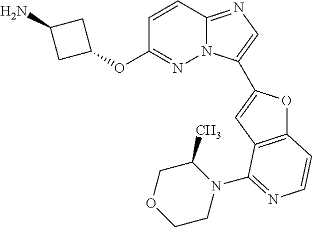 [SMILES: C[C@@H]1COCCN1c1nccc2oc(-c3cnc4ccc(O[C@H]5C[C@H](N)C5)nn34)cc12]

Step 1: To 141 mg (0.37 mmol) tert-butyl {trans-3-[(3-bromoimidazo[1,2-b]pyridazin-6-yl)oxy]cyclobutyl}-carbamate were added 323 mg (0.74 mmol) {4-[(3R)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Water was added. The mixture was extracted with dichloromethane. The organic layer was dried over sodium sulfate and evaporated. The crude product (432 mg) was used without further purification in the subsequent step 2.

Step 2: 4 mL Dichlormethane were added to the crude material from step 1. 2 mL TFA were added and the mixture was stirred at room temperature for 15 min.

Water was added. 2 mL Aqueous ammonia (25% in water) were added. The mixture was extracted with dichloromethane. The organic layer was dried over sodium sulfate and evaporated. The crude product was digested in methanol to give 46 mg of the title compound.

¹H-NMR (400 MHz, DMSO-d₆), δ [ppm]=1.25 (2H), 2.12-2.26 (2H), 3.38-3.70 (3H), 3.73-3.86 (2H), 3.99-4.10 (2H), 4.56 (1H), 5.34-5.43 (1H), 6.99 (1H), 7.06 (1H), 7.57 (1H), 8.01 (1H), 8.08-8.16 (2H).

LC-MS (Method 13): Rₜ=0.55 min; MS (ESIpos) m/z=421 [M+H]⁺.

### Example III-108

### 2-{6-[(trans-3-Aminocyclobutyl)oxy]imidazo[1,2-b]pyridazin-3-yl}-N-(2-tert-butoxyethyl)-N-ethylfuro[3,2-c]pyridin-4-amine

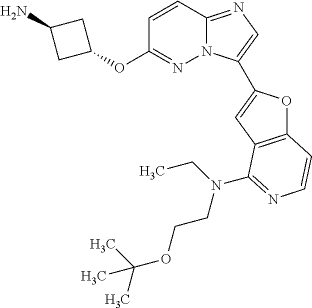 [SMILES: CCN(CCOC(C)(C)C)c1nccc2oc(-c3cnc4ccc(O[C@H]5C[C@H](N)C5)nn34)cc12]

To a stirred suspension of tert-butyl {trans-3-[(3-{4-[(2-tert-butoxyethyl)(ethyl)-amino]furo[3,2-c]pyridin-2-yl}imidazo[1,2-b]pyridazin-6-yl)oxy]cyclobutyl}carbamate (120 mg) in dichloromethane (7 mL) was added TFA (0.2 mL). The mixture was stirred at room temperature for 16 h. The solvent was removed in vacuum. A saturated solution of sodium bicarbonate was added and the mixture was extracted with a mixture of dichloromethane and methanol. The organic phase was dried (sodium sulfate) and the solvent was removed in vacuum. Aminophase silicagel chromatography gave 50 mg of the title compound.

¹H-NMR (400 MHz, DMSO-d₆), δ [ppm]=1.04-1.11 (9H), 1.29 (3H), 1.86 (2H), 2.15-2.25 (2H), 2.33-2.44 (2H), 3.53-3.69 (3H), 3.73-3.86 (4H), 5.39-5.48 (1H), 6.89 (1H), 6.98 (1H), 7.60 (1H), 7.94 (1H), 8.07 (1H), 8.13 (1H).

### Example III-109

### trans-3-[(3-{4-[(3S)-3-Methylmorpholin-4-yl]furo[3,2-c]pyridin-2-yl}imidazo[1,2-b]pyridazin-6-yl)oxy]cyclobutanamine

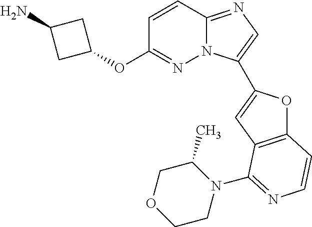 [SMILES: C[C@H]1COCCN1c1nccc2oc(-c3cnc4ccc(O[C@H]5C[C@H](N)C5)nn34)cc12]

Step 1: To 141 mg (0.37 mmol) tert-butyl {trans-3-[(3-bromoimidazo[1,2-b]pyridazin-6-yl)oxy]cyclobutyl}carbamate were added 323 mg (0.74 mmol) {4-[(3S)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 17 h.

Half-saturated brine was added. The mixture was extracted with dichloromethane. The organic layer was dried over sodium sulfate and evaporated. The crude product (302 mg) was used without further purification in the subsequent step 2.

Step 2: 4 mL Dichlormethane were added to the crude material from step 1. 2 mL TFA were added and the mixture was stirred at room temperature for 10 min.

Water was added. 2 mL Aqueous ammonia (25% in water) were added. The mixture was extracted with dichloromethane. The organic layer was dried over sodium sulfate and evaporated. The crude product was purified by HPLC to give 64 mg of the title compound.

¹H-NMR (400 MHz, DMSO-d₆), δ [ppm]=1.27 (3H), 2.22-2.35 (2H), 3.56-3.89 (6H), 4.01-4.13 (2H), 4.59 (1H), 5.41 (1H), 7.02 (1H), 7.09 (1H), 7.59 (1H), 8.04 (1H), 8.10-8.20 (2H), 8.28 (1H).

LC-MS (Method 13): Rₜ=0.55 min; MS (ESIpos) m/z=421 [M+H]⁺.

### Example III-110

### trans-3-[(3-{4-[(2S)-2-Methylmorpholin-4-yl]furo[3,2-c]pyridin-2-yl}imidazo[1,2-b]pyridazin-6-yl)oxy]cyclobutanamine

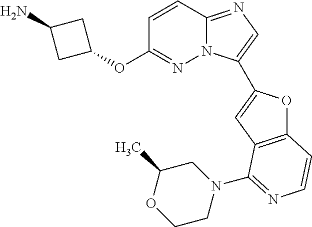 [SMILES: C[C@H]1CN(c2nccc3oc(-c4cnc5ccc(O[C@H]6C[C@H](N)C6)nn45)cc23)CCO1]

Step 1: To 141 mg (0.37 mmol) tert-butyl {trans-3-[(3-bromoimidazo-[1,2-b]pyridazin-6-yl)oxy]cyclobutyl}-carbamate were added 312 mg (0.74 mmol) {4-[(2S)-3-methylmorpholin-4-yl]furo[3,2-c]pyridin-2-yl}boronic acid, 17 mg (15 μmol) tetrakis(triphenylphosphin)-palladium(0), and 0.55 mL of a 2 M aqueous solution of sodium carbonate. The mixture was stirred at 110° C. for 18 h.

Water was added. The mixture was extracted with dichloromethane. The organic layer was dried over sodium sulfate and evaporated. The crude product (332 mg) was used without further purification in the subsequent step 2.

Step 2: 4 mL Dichlormethane were added to the crude material from step 1. 2 mL TFA were added and the mixture was stirred at room temperature for 10 min.

Water was added. 2 mL Aqueous ammonia (25% in water) were added. The mixture was extracted with dichloromethane. The organic layer was dried over sodium sulfate and evaporated. The crude product was purified by flash chromatography to give 98 mg of the title compound.

¹H-NMR (400 MHz, DMSO-d₆), δ [ppm]=1.20 (3H), 2.21-2.34 (2H), 2.75-2.87 (1H), 3.25 (3H), 3.63-3.79 (3H), 3.99-4.08 (1H), 4.12-4.22 (1H), 4.27 (1H), 5.34-5.44 (1H), 7.03 (1H), 7.12 (1H), 7.58 (1H), 8.03 (1H), 8.10-8.19 (2H).

LC-MS (Method 12): Rₜ=0.58 min; MS (ESIpos) m/z=421 [M+H]⁺.

### Example III-111

### 2-(6-{[(1S,2S)-1-Amino-2,3-dihydro-1H-inden-2-yl]oxy}imidazo[1,2-b]pyridazin-3-yl)-N-ethyl-N-(2-methoxyethyl)furo[3,2-c]pyridin-4-amine

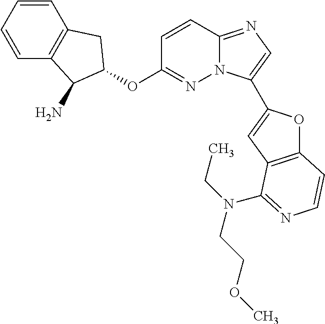 [SMILES: CCN(CCOC)c1nccc2oc(-c3cnc4ccc(O[C@H]5Cc6ccccc6[C@@H]5N)nn34)cc12]

To a stirred solution of (1S,2S)-2-[(3-bromoimidazo[1,2-b]pyridazin-6-yl)oxy]-2,3-dihydro-1H-inden-1-amine (102 mg) in 1-propanol (5 mL) was added 2M potassium carbonate solution (0.44 mL), crude {4-[Ethyl(2-methoxyethyl)amino]furo[3,2-c]pyridin-2-yl}boronic acid (82% w/w; 190 mg), triphenylphosphine (7.75 mg) and PdCl₂(PPh₃)₂ (20.7 mg). The mixture was heated to reflux for 2 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography followed by silicagel chromatography gave a solid that was triturated with hexane to give 52 mg of the title compound.

¹H-NMR (300 MHz, DMSO-d₆), δ [ppm]=0.97 (3H), 2.18 (2H), 2.93 (1H), 3.13 (3H), 3.37-3.45 (2H), 3.46-3.80 (5H), 4.46 (1H), 5.36 (1H), 6.90 (1H), 7.03 (1H), 7.17-7.31 (3H), 7.39 (1H), 7.63 (1H), 7.92 (1H), 8.11 (1H), 8.17 (1H).

### Example III-112

### 2-(6-{[(1R,2S)-1-Amino-2,3-dihydro-1H-inden-2-yl]oxy}imidazo[1,2-b]pyridazin-3-yl)-N-ethyl-N-(2-methoxyethyl)furo[3,2-c]pyridin-4-amine

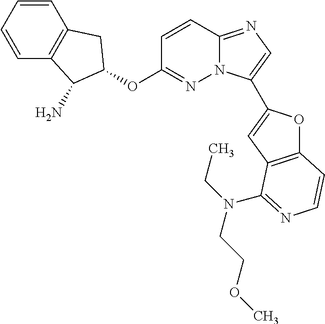 [SMILES: CCN(CCOC)c1nccc2oc(-c3cnc4ccc(O[C@H]5Cc6ccccc6[C@H]5N)nn34)cc12]

To a stirred solution of (1R,2S)-2-[(3-bromoimidazo[1,2-b]pyridazin-6-yl)oxy]-2,3-dihydro-1H-inden-1-amine (104 mg) in 1-propanol (5 mL) was added 2M potassium carbonate solution (0.45 mL), crude {4-[Ethyl(2-methoxyethyl)amino]furo[3,2-c]pyridin-2-yl}boronic acid (82% w/w; 195 mg), triphenylphosphine (7.90 mg) and PdCl₂(PPh₃)₂ (21.1 mg). The mixture was heated to reflux for 2 h. The warm mixture was filtered and the solvent was removed in vacuum. A half-saturated solution of sodium bicarbonate was added and the mixture was extracted with a mixture of dichloromethane and methanol. The organic phase was dried (sodium sulfate) and the solvent was removed in vacuum. Aminophase silicagel chromatography gave a solid that was triturated with hexane to give 50 mg of the title compound.

¹H-NMR (400 MHz, Pyr-d₅), δ [ppm]=1.24 (3H), 2.27 (2H), 3.25 (3H), 3.31-3.50 (2H), 3.58-3.72 (2H), 3.73-3.86 (2H), 3.95 (2H), 4.85 (1H), 5.82 (1H), 6.67 (1H), 7.00 (1H), 7.25-7.39 (3H), 7.64-7.80 (2H), 7.96 (1H), 8.21 (1H), 8.39 (1H).

### Example III-113

### (6S)-6-{[(3-{4-[Ethyl(2-methoxyethyl)amino]furo[3,2-c]pyridin-2-yl}imidazo[1,2-b]pyridazin-6-yl)oxy]methyl}piperazin-2-one

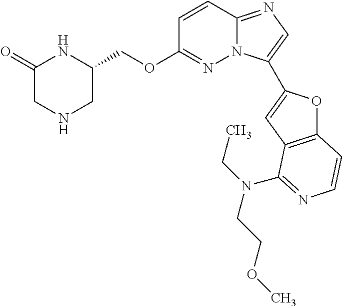 [SMILES: CCN(CCOC)c1nccc2oc(-c3cnc4ccc(OC[C@@H]5CNCC(=O)N5)nn34)cc12]

Step 1: In an ice bath 52 mg (1.3 mmol) sodium hydride (60% dispersion in mineral oil) were dispensed in 6 mL of anhydrous tetrahydrofurane. 300 mg (1.3 mmol) (65)-4-(2,2-dimethylpropanoyl)-6-(hydroxymethyl)piperazin-2-one were slowly added. After complete addition, stirring at 0° C. was continued for 10 min. 363 mg (0.98 mmol) of 2-(6-chloroimidazo[1,2-b]pyridazin-3-yl)-N-ethyl-N-(2-methoxy-ethyl)furo[3,2-c]pyridin-4-amine were added, the ice bath removed and the resulting mixture was stirred for 24 h at 40° C.

Brine was added. The mixture was extracted with ethyl acetate. The organic layer was dried over magnesium sulfate and evaporated to give a crude product which was used in step 2.

Step 2: 10 mL dichlormethane were added the crude material from step 1. 7.5 mL TFA were added and the mixture was stirred at room temperature for 24 h.

Aqueous ammonia was added until basic pH was reached. The mixture was extracted with dichloromethane. The organic layer was dried over sodium sulfate and evaporated. The crude product was purified by HPLC to give 92 mg of the title compound.

¹H-NMR (400 MHz, DMSO-d6), δ [ppm]=1.27 (3H) 2.92 (1H) 3.01 (1H) 3.21 (2H) 3.62 (2H) 3.72-3.91 (5H) 4.38 (1H) 4.58 (1H) 6.92-6.96 (1H) 7.07 (1H) 7.70 (1H) 7.86 (1H) 7.97 (1H) 8.13 (1H) 8.20 (1H).

LC-MS (Method 12): Rₜ=0.53 min; MS (ESIpos) m/z=466 [M+H]⁺.

The following examples have been prepared in analogy to the examples above, using starting materials which were either commercially available or which have been prepared by methods described in the literature.

| | | | MW | Retention | |
|---|---|---|---|---|---|
| Exam- | | | found | time | HPLC |
| ple | Structure | Name | [M + H]⁺ | [min] | Method |
| III-114 | 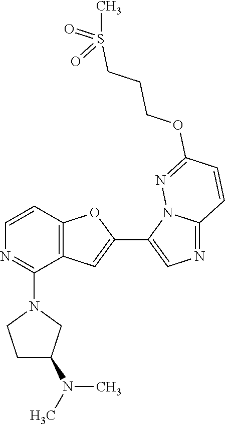 [SMILES: CN(C)[C@H]1CCN(c2nccc3oc(-c4cnc5ccc(OCCCS(C)(=O)=O)nn45)cc23)C1] | (3S)-N,N-dimethyl-1-(2- {6-[3- (methylsulfonyl)propoxy] imidazo[1,2-b]pyridazin- 3-yl}furo[3,2-c]pyridin-4- yl)pyrrolidin-3-amine | 485 | 0.94 | 14 |
| III-115 | 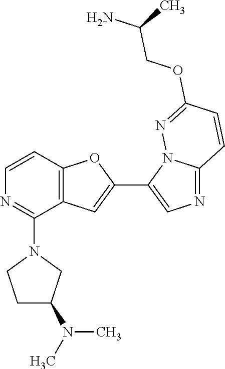 [SMILES: C[C@H](N)COc1ccc2ncc(-c3cc4c(N5CC[C@H](N(C)C)C5)nccc4o3)n2n1] | (3S)-1-[2-(6-{[(2S)-2- aminopropyl]oxy}imidazo [1,2-b]pyridazin-3- yl)furo[3,2-c]pyridin-4- yl]-N,N- dimethylpyrrolidin-3- amine | 422 | 0.98 | 18 |
| III-116 | 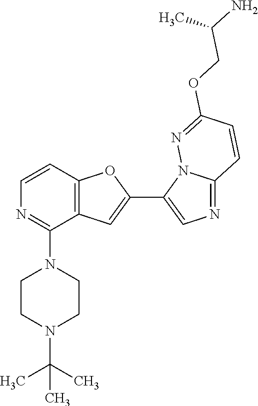 [SMILES: C[C@H](N)COc1ccc2ncc(-c3cc4c(N5CCN(C(C)(C)C)CC5)nccc4o3)n2n1] | (2S)-1-({3-[4-(4-tert- butylpiperazin-1- yl)furo[3,2-c]pyridin-2- yl]imidazo[1,2-b]pyridazin- 6-yl}oxy)propan-2-amine | 450 | 1.16 | 15 |
| III-117 | 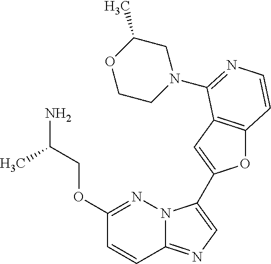 [SMILES: C[C@H](N)COc1ccc2ncc(-c3cc4c(N5CCO[C@H](C)C5)nccc4o3)n2n1] | (2S)-1-[(3-{4-[(2R)-2- methylmorpholin-4- yl]furo[3,2-c]pyridin-2- yl}imidazo[1,2-b]pyridazin- 6-yl)oxy]propan-2-amine | 409 | 0.58 | 16 |
| III-118 | 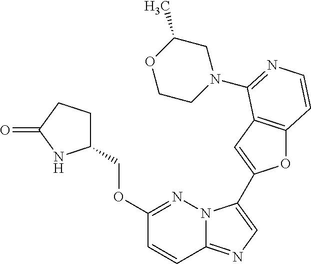 [SMILES: C[C@@H]1CN(c2nccc3oc(-c4cnc5ccc(OC[C@H]6CCC(=O)N6)nn45)cc23)CCO1] | (5R)-5-{[(3-{4-[(2R)-2- methylmorpholin-4- yl]furo[3,2-c]pyridin-2- yl}imidazo[1,2-b]pyridazin- 6-yl)oxy]methyl}pyrrolidin- 2-one | 449 | 0.7 | 17 |
| III-119 | 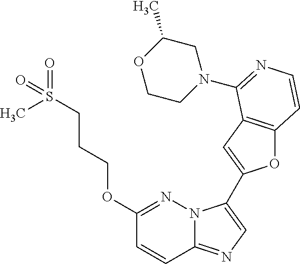 [SMILES: C[C@@H]1CN(c2nccc3oc(-c4cnc5ccc(OCCCS(C)(=O)=O)nn45)cc23)CCO1] | 3-{4-[(2R)-2- methylmorpholin-4- yl]furo[3,2-c]pyridin-2- yl}-6-[3- (methylsulfonyl)propoxy] imidazo[1,2-b]pyridazine | 472 | 0.72 | 17 |
| III-120 | 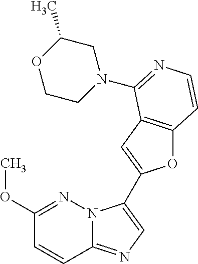 [SMILES: COc1ccc2ncc(-c3cc4c(N5CCO[C@H](C)C5)nccc4o3)n2n1] | 6-methoxy-3-{4-[(2R)-2- methylmorpholin-4- yl]furo[3,2-c]pyridin-2- yl}imidazo[1,2-b]pyridazine | 366 | 0.79 | 17 |
| III-121 | 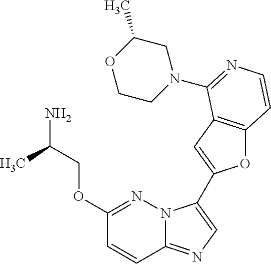 [SMILES: C[C@@H]1CN(c2nccc3oc(-c4cnc5ccc(OC[C@@H](C)N)nn45)cc23)CCO1] | (2R)-1-[(3-{4-[(2R)-2- methylmorpholin-4- yl]furo[3,2-c]pyridin-2- yl}imidazo[1,2-b]pyridazin- 6-yl)oxy]propan-2-amine | 409 | 0.59 | 16 |
| III-122 | 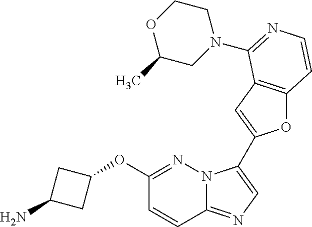 [SMILES: C[C@@H]1CN(c2nccc3oc(-c4cnc5ccc(O[C@H]6C[C@H](N)C6)nn45)cc23)CCO1] | trans-3-[(3-{4-[(2R)-2- methylmorpholin-4- yl]furo[3,2-c]pyridin-2- yl}imidazo[1,2-b]pyridazin- 6-yl)oxy]cyclobutanamine | 421 | 0.59 | 16 |
| III-123 | 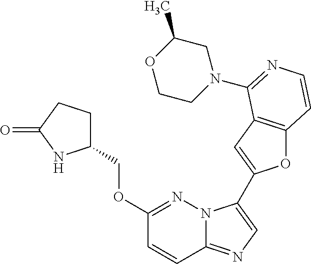 [SMILES: C[C@H]1CN(c2nccc3oc(-c4cnc5ccc(OC[C@H]6CCC(=O)N6)nn45)cc23)CCO1] | (5R)-5-{[(3-{4-[(2S)-2- methylmorpholin-4- yl]furo[3,2-c]pyridin-2- yl}imidazo[1,2-b]pyridazin- 6-yl)oxy]methyl}pyrrolidin- 2-one | 449 | 0.69 | 13 |
| III-124 | 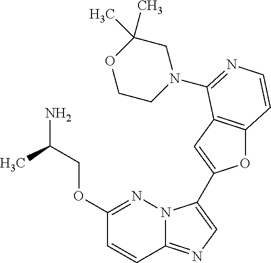 [SMILES: C[C@@H](N)COc1ccc2ncc(-c3cc4c(N5CCOC(C)(C)C5)nccc4o3)n2n1] | (2R)-1-({3-[4-(2,2- dimethylmorpholin-4- yl)furo[3,2-c]pyridin-2- yl]imidazo[1,2-b]pyridazin- 6-yl}oxy)propan-2-amine | 423 | 0.59 | 13 |
| III-125 | 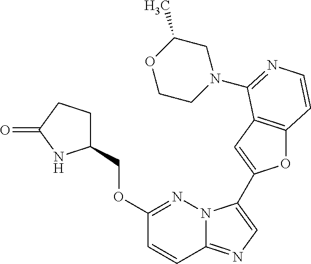 [SMILES: C[C@@H]1CN(c2nccc3oc(-c4cnc5ccc(OC[C@@H]6CCC(=O)N6)nn45)cc23)CCO1] | (5S)-5-{[(3-{4-[(2R)-2- methylmorpholin-4- yl]furo[3,2-c]pyridin-2- yl}imidazo[1,2-b]pyridazin- 6-yl)oxy]methyl}pyrrolidin- 2-one | 449 | 0.71 | 13 |
| III-126 | 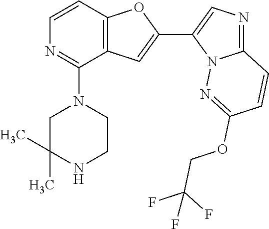 [SMILES: CC1(C)CN(c2nccc3oc(-c4cnc5ccc(OCC(F)(F)F)nn45)cc23)CCN1] | 3-[4-(3,3- dimethylpiperazin-1- yl)furo[3,2-c]pyridin-2-yl]- 6-(2,2,2- trifluoroethoxy)imidazo [1,2-b]pyridazine | 447 | 0.81 | 17 |
| III-127 | 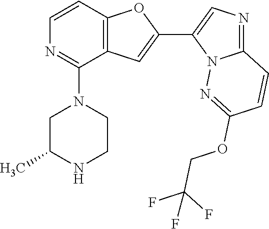 [SMILES: C[C@@H]1CN(c2nccc3oc(-c4cnc5ccc(OCC(F)(F)F)nn45)cc23)CCN1] | 3-{4-[(3R)-3- methylpiperazin-1- yl]furo[3,2-c]pyridin-2-yl}- 6-(2,2,2- trifluoroethoxy)imidazo [1,2-b]pyridazine | 433 | 0.77 | 17 |
| III-128 | 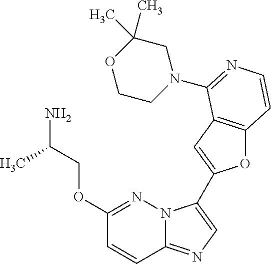 [SMILES: C[C@H](N)COc1ccc2ncc(-c3cc4c(N5CCOC(C)(C)C5)nccc4o3)n2n1] | (2S)-1-({3-[4-(2,2- dimethylmorpholin-4- yl)furo[3,2-c]pyridin-2- yl]imidazo[1,2-b]pyridazin- 6-yl}oxy)propan-2-amine | 423 | 0.59 | 13 |
| III-129 | 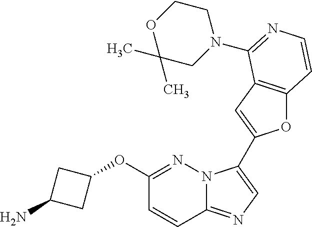 [SMILES: CC1(C)CN(c2nccc3oc(-c4cnc5ccc(O[C@H]6C[C@H](N)C6)nn45)cc23)CCO1] | trans-3-({3-[4-(2,2- dimethylmorpholin-4- yl)furo[3,2-c]pyridin-2- yl]imidazo[1,2-b]pyridazin- 6-yl}oxy)cyclobutanamine | 435 | 0.6 | 13 |
| III-130 | 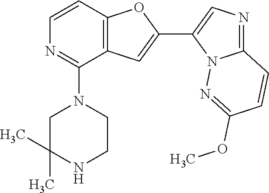 [SMILES: COc1ccc2ncc(-c3cc4c(N5CCNC(C)(C)C5)nccc4o3)n2n1] | 3-[4-(3,3- dimethylpiperazin-1- yl)furo[3,2-c]pyridin-2- yl]-6-methoxy- imidazo[1,2-b]pyridazine | 379 | 0.72 | 17 |
| III-131 | 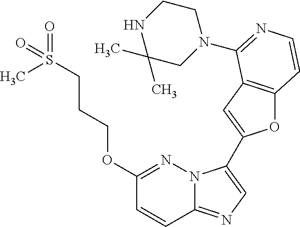 [SMILES: CC1(C)CN(c2nccc3oc(-c4cnc5ccc(OCCCS(C)(=O)=O)nn45)cc23)CCN1] | 3-[4-(3,3-dimethylpiperazin- 1-yl)furo[3,2-c]pyridin-2- yl]-6-[3- (methylsulfonyl)propoxy] imidazo[1,2-b]pyridazine | 485 | 0.68 | 17 |
| III-132 | 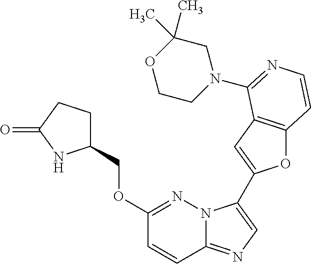 [SMILES: CC1(C)CN(c2nccc3oc(-c4cnc5ccc(OC[C@@H]6CCC(=O)N6)nn45)cc23)CCO1] | (5S)-5-[({3-[4-(2,2- dimethylmorpholin-4- yl)furo[3,2-c]pyridin-2- yl]imidazo[1,2-b]pyridazin- 6-yl}oxy)methyl]pyrrolidin- 2-one | 463 | 0.78 | 12 |
| III-133 | 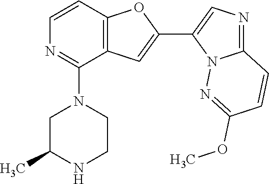 [SMILES: COc1ccc2ncc(-c3cc4c(N5CCN[C@@H](C)C5)nccc4o3)n2n1] | 6-methoxy-3-{4-[(3S)-3- methylpiperazin-1- yl]furo[3,2-c]pyridin-2- yl}imidazo[1,2-b]pyridazine | 365 | 0.68 | 13 |
| III-134 | 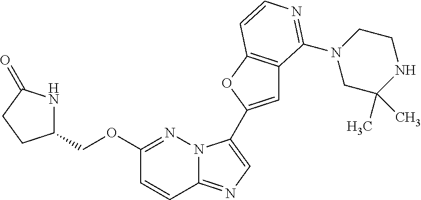 [SMILES: CC1(C)CN(c2nccc3oc(-c4cnc5ccc(OC[C@@H]6CCC(=O)N6)nn45)cc23)CCN1] | (5S)-5-[({3-[4-(3,3- dimethylpiperazin-1- yl)furo[3,2-c]pyridin-2- yl]imidazo[1,2-b]pyridazin- 6-yl}oxy)methyl]pyrrolidin- 2-one | 462 | 0.67 | 14 |
| III-135 | 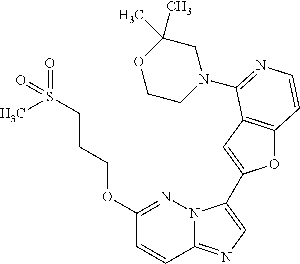 [SMILES: CC1(C)CN(c2nccc3oc(-c4cnc5ccc(OCCCS(C)(=O)=O)nn45)cc23)CCO1] | 3-[4-(2,2- dimethylmorpholin-4- yl)furo[3,2-c]pyridin-2- yl]-6-[3- (methylsulfonyl)propoxy] imidazo[1,2-b]pyridazine | 486 | 0.76 | 12 |
| III-136 | 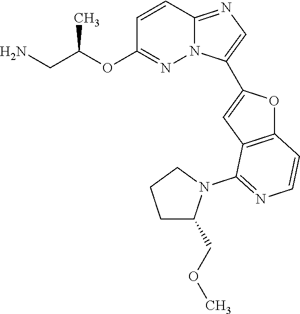 [SMILES: COC[C@@H]1CCCN1c1nccc2oc(-c3cnc4ccc(O[C@H](C)CN)nn34)cc12] | (2R)-2-[(3-{4-[(2S)-2- (methoxymethyl)pyrrolidin- 1-yl]furo[3,2-c]pyridin-2- yl}imidazo[1,2-b]pyridazin- 6-yl)oxy]propan-1-amine | 423 | 1.11 | 15 |
| III-137 | 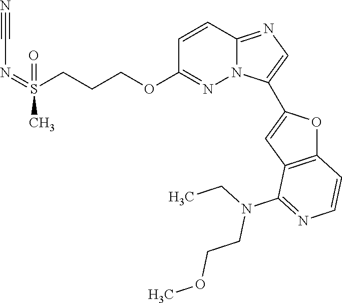 [SMILES: CCN(CCOC)c1nccc2oc(-c3cnc4ccc(OCCC[S@@](C)(=O)=NC#N)nn34)cc12] | [{3-[(3-{4-[ethyl(2- methoxyethyl)ami- no]furo[3,2-c]pyridin-2- yl}imidazo[1,2-b]pyridazin-6- yl)oxy]propyl}(methyl)oxido- λ⁶-sulfanylidene]cyanamide | 498 | 0.75 | 13 |
| III-138 | 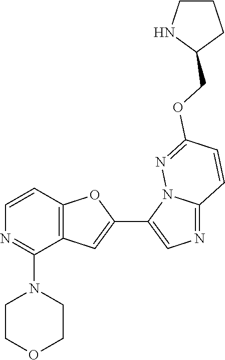 [SMILES: c1cc2oc(-c3cnc4ccc(OC[C@@H]5CCCN5)nn34)cc2c(N2CCOCC2)n1] | 3-[4-(morpholin-4- yl)furo[3,2-c]pyridin-2-yl]-6- [(2S)-pyrrolidin-2-ylmeth- oxy]imidazo[1,2-b]pyridazine | 421 | 1.07 | 12 |
| III-139 | 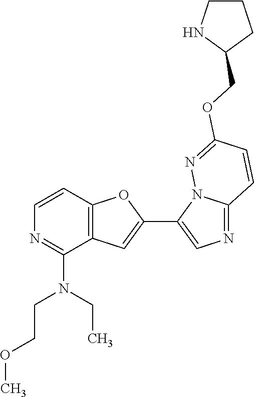 [SMILES: CCN(CCOC)c1nccc2oc(-c3cnc4ccc(OC[C@@H]5CCCN5)nn34)cc12] | N-ethyl-N-(2-methoxyethyl)- 2-{6-[(2S)- pyrrolidin-2-ylmeth- oxy]imidazo[1,2-b]pyridazin- 3-yl}furo[3,2-c]pyridin-4- amine | 437 | 1.25 | 15 |
| III-140 | 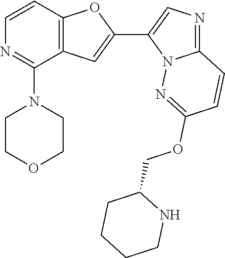 [SMILES: c1cc2oc(-c3cnc4ccc(OC[C@H]5CCCCN5)nn34)cc2c(N2CCOCC2)n1] | 3-[4-(morpholin-4- yl)furo[3,2-c]pyridin-2- yl]-6-(piperidin-2-ylmeth- oxy)imidazo[1,2-b]pyridazine | 435 | 1.12 | 15 |
| III-141 | 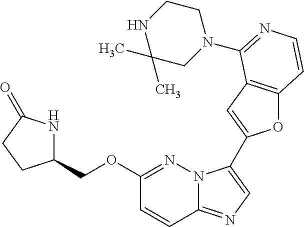 [SMILES: CC1(C)CN(c2nccc3oc(-c4cnc5ccc(OC[C@H]6CCC(=O)N6)nn45)cc23)CCN1] | (5R)-5-[({3-[4-(3,3- dimethylpiperazin-1- yl)furo[3,2-c]pyridin-2- yl]imidazo[1,2-b]pyridazin- 6-yl}oxy)methyl]pyrrolidin- 2-one | 462 | 0.68 | 12 |
| III-142 | 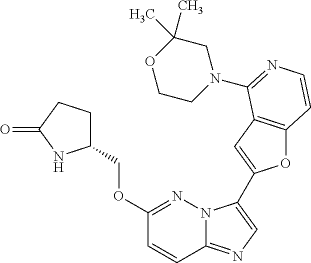 [SMILES: CC1(C)CN(c2nccc3oc(-c4cnc5ccc(OC[C@H]6CCC(=O)N6)nn45)cc23)CCO1] | (5R)-5-[({3-[4-(2,2- dimethylmorpholin-4- yl)furo[3,2-c]pyridin-2- yl]imidazo[1,2-b]pyridazin-6- yl}oxy)methyl]pyrrolidin- 2-one | 463 | 0.74 | 12 |
| III-143 | 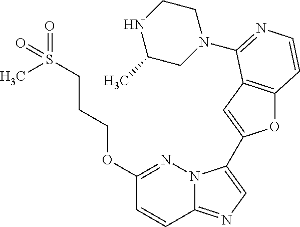 [SMILES: C[C@H]1CN(c2nccc3oc(-c4cnc5ccc(OCCCS(C)(=O)=O)nn45)cc23)CCN1] | 3-{4-[(3S)-3- methylpiperazin-1- yl]furo[3,2-c]pyridin-2- yl}-6-[3- (methylsulfonyl)propoxy] imidazo[1,2-b]pyridazine | 471 | 0.68 | 13 |
| III-144 | 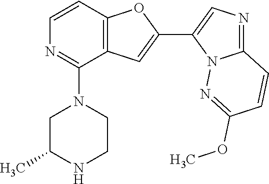 [SMILES: COc1ccc2ncc(-c3cc4c(N5CCN[C@H](C)C5)nccc4o3)n2n1] | 6-methoxy-3-{4-[(3R)-3- methylpiperazin-1- yl]furo[3,2-c]pyridin-2- yl}imidazo[1,2-b]pyridazine | 365 | 0.68 | 13 |
| III-145 | 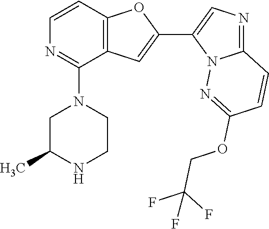 [SMILES: C[C@H]1CN(c2nccc3oc(-c4cnc5ccc(OCC(F)(F)F)nn45)cc23)CCN1] | 3-{4-[(3S)-3- methylpiperazin-1- yl]furo[3,2-c]pyridin-2- yl}-6-(2,2,2-trifluoroethox- y)imidazo[1,2-b]pyridazine | 433 | 0.73 | 11 |
| III-146 | 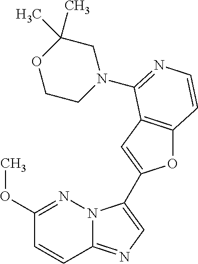 [SMILES: COc1ccc2ncc(-c3cc4c(N5CCOC(C)(C)C5)nccc4o3)n2n1] | 3-[4-(2,2- dimethylmorpholin-4- yl)furo[3,2-c]pyridin-2- yl]-6-methoxy- imidazo[1,2-b]pyridazine | 380 | 0.86 | 12 |

### Example IV-1

### 1-[(2S)-2-(2-{[3-(Furo[3,2-c]pyridin-2-yl)imidazo[1,2-b]pyridazin-6-yl]oxy}ethyl)pyrrolidin-1-yl]ethanone

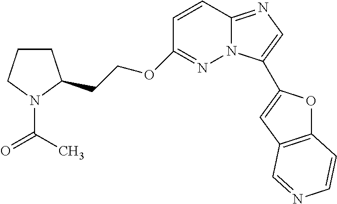 [SMILES: CC(=O)N1CCC[C@H]1CCOc1ccc2ncc(-c3cc4cnccc4o3)n2n1]

To 80 mg (0.23 mmol) 3-(furo[3,2-c]pyridin-2-yl)-6-{2-[(2S)-pyrrolidin-2-yl]ethoxy}-imidazo[1,2-b]-pyridazine (used as crude product) in 3 mL THF were added 74 μL (0.92 mmol) pyridine and 86 μL (0.92 mmol) acetic anhydride. The mixture was stirred a for 3 h at room temperature.

50 μL of water were added and the mixture was stirred for 5 min. Saturated aqueous sodium hydrogen carbonate solution was added and the mixture was extracted with ethyl acetate. The organic layer was dried over sodium sulfate and evaporated. The obtained crude product was purified by flash chromatography followed by HPLC to give 34 mg of the title compound as solid material.

¹H-NMR (300 MHz, DMSO-d₆), δ [ppm]=1.75-2.04 (6H), 2.13-2.29 (1H), 3.43 (2H), 3.96-4.25 (1H), 4.43-4.60 (2H), 7.02 (1H), 7.64-7.78 (2H), 8.11-8.22 (2H), 8.46 (1H), 9.01 (1H).

LC-MS (Method 21): Rₜ=0.69 min; MS (ESIpos) m/z=392 [M+H]⁺.

### Example IV-2

### 5-(2-{[3-(4-Methoxyfuro[3,2-c]pyridin-2-yl)imidazo[1,2-b]pyridazin-6-yl]oxy}-ethyl)pyrrolidin-2-one

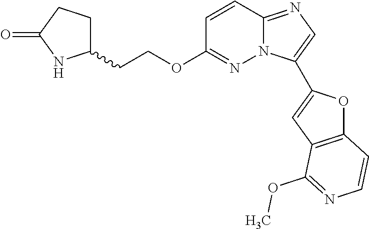 [SMILES: COc1nccc2oc(-c3cnc4ccc(OCCC5CCC(=O)N5)nn34)cc12]

At 0-5° C. 86 mg (0.67 mmol) 5-(2-hydroxyethyl)pyrrolidin-2-one were added to 26.6 mg (0.67 mmol) sodium hydride (60% in mineral oil) in 4.5 mL anhydrous DMF. After 5 min of stirring on the ice bath, 100 mg (0.33 mmol) 6-chloro-3-(4-methoxyfuro[3,2-c]pyridin-2-yl)imidazo[1,2-b]pyridazine were added. The ice bath was removed and it was stirred 3 hours at room temperature.

The reaction mixture was poured into half saturated ammonium chloride solution. It was extracted four times with ethyl acetate. The combined organic phases were washed with brine, dried over magnesium sulfate and concentrated. The residue was purified by HPLC yielding 4.5 mg (3%) product.

¹H-NMR (300 MHz, DMSO-d₆), δ [ppm]=1.67-1.81 (1H), 1.95-2.07 (2H), 2.10-2.28 (3H), 3.73-3.83 (1H), 4.01 (3H), 4.46-4.62 (2H), 7.02 (1H), 7.35 (1H), 7.47 (1H), 7.87 (1H), 8.03 (1H), 8.11-8.18 (2H).

LC-MS (Method 20): Rₜ=0.94 min; MS (ESIpos) m/z=394 [M+H]⁺.

### Example IV-3

### (5S)-5-({[3-(4-Methoxyfuro[3,2-c]pyridin-2-yl)imidazo[1,2-b]pyridazin-6-yl]-oxy}methyl)pyrrolidin-2-one

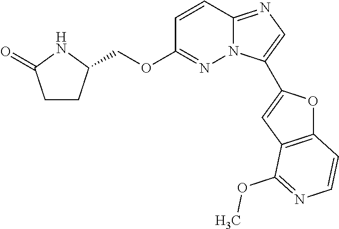 [SMILES: COc1nccc2oc(-c3cnc4ccc(OC[C@@H]5CCC(=O)N5)nn34)cc12]

In an ice bath, 78 mg (0.67 mmol) (5S)-5-(hydroxymethyl)pyrrolidin-2-one were added to 27 mg (0.67 mmol) sodium hydride (60% in mineral oil) in a mixture of 4 mL anhydrous THF and 2 mL anhydrous DMF. After 15 min of stirring on the ice bath, 100 mg (0.33 mmol) of 6-chloro-3-(4-methoxyfuro[3,2-c]pyridin-2-yl)imidazo[1,2-b]pyridazine were added. The ice bath was removed and the mixture was stirred for 19 h at 40° C.

The reaction mixture was poured into water. The precipitate was filtered off, washed with water and dried in vacuum. The crude material was digested in methanol to give 55 mg of the title compound as solid material.

¹H-NMR (400 MHz, DMSO-d₆): δ [ppm]=1.93-2.03 (1H), 2.15-2.41 (3H), 4.04-4.13 (3H), 4.42 (1H), 4.52 (1H), 7.08 (1H), 7.39 (1H), 7.50 (1H), 7.91 (1H), 8.08 (1H), 8.18-8.22 (2H).

LC-MS (Method 21): Rₜ=0.92 min; MS (ESIpos) m/z=380 [M+H]⁺.

### Example IV-4

### (5R)-5-[({3-[4-(Propan-2-yloxy)furo[3,2-c]pyridin-2-yl]imidazo[1,2-b]pyridazin-6-yl}oxy)methyl]pyrrolidin-2-one

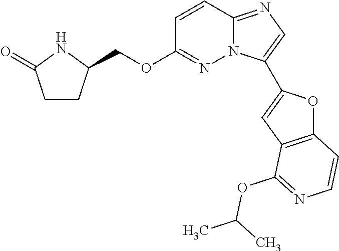 [SMILES: CC(C)Oc1nccc2oc(-c3cnc4ccc(OC[C@H]5CCC(=O)N5)nn34)cc12]

In an ice bath, 64 mg (0.55 mmol) (5R)-5-(hydroxymethyl)pyrrolidin-2-one were added to 22 mg (0.55 mmol) sodium hydride (60% in mineral oil) in a mixture of 2.7 mL anhydrous THF and 1 mL anhydrous DMF. After 15 min of stirring on the ice bath, 90 mg (0.27 mmol) of 6-chloro-3-[4-(propan-2-yloxy)furo[3,2-c]pyridin-2-yl]imidazo[1,2-b]pyridazine were added. The ice bath was removed and the mixture was stirred for 18 h at 40° C.

The reaction mixture was poured into water and extracted with ethyl acetate. The organic layer was dried over sodium sulfate and evaporated. The crude material was purified by HPLC to yield 49 mg of the title compound as solid material.

¹H-NMR (400 MHz, DMSO-d₆): δ [ppm]=1.40 (6H), 1.88-2.00 (1H), 2.15-2.40 (3H), 4.10-4.18 (m, 1H), 4.39 (1H), 4.57 (1H), 5.47 (1H), 7.07 (1H), 7.33 (1H), 7.47 (1H), 7.95 (1H), 8.04 (1H), 8.18 (1H), 8.20 (1H).

LC-MS (Method 21): Rₜ=1.09 min; MS (ESIpos) m/z=408 [M+H]⁺.

### Example IV-5

### (5R)-5-[({3-[4-(2,2-Dimethylpropoxy)furo[3,2-c]pyridin-2-yl]imidazo[1,2-b]-pyridazin-6-yl}oxy)methyl]pyrrolidin-2-one

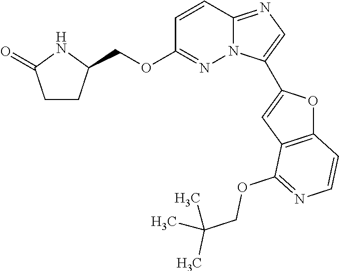 [SMILES: CC(C)(C)COc1nccc2oc(-c3cnc4ccc(OC[C@H]5CCC(=O)N5)nn34)cc12]

In an ice bath, 59 mg (0.50 mmol) (5R)-5-(hydroxymethyl)pyrrolidin-2-one were added to 20 mg (0.50 mmol) sodium hydride (60% in mineral oil) in a mixture of 3 mL anhydrous THF and 1 mL anhydrous DMF. After 15 min of stirring on the ice bath, 90 mg (0.25 mmol) of 6-chloro-3-[4-(2,2-dimethylpropoxy)furo[3,2-c]pyridin-2-yl]imidazo[1,2-b]pyridazine were added. The ice bath was removed and the mixture was stirred for 19 h at 40° C.

The reaction mixture was poured into water and extracted with dichlormethane and ethyl acetate. The combined organic layers were dried over sodium sulfate and evaporated. The crude material was digested in methanol to yield 59 mg of the title compound as solid material.

¹H-NMR (400 MHz, DMSO-d₆): δ [ppm]=1.07 (9H), 1.80-1.90 (1H), 2.18-2.36 (3H), 4.04-4.11 (1H), 4.15 (2H), 4.36 (1H), 4.54 (1H), 7.06 (1H), 7.36 (1H), 7.56 (1H), 7.99 (1H), 8.03 (1H), 8.19 (1H), 8.21 (1H).

LC-MS (Method 21): Rₜ=1.24 min; MS (ESIpos) m/z=436 [M+H]⁺.

### Example IV-6

### (5R)-5-({[3-(4-Methoxyfuro[3,2-c]pyridin-2-yl)imidazo[1,2-b]pyridazin-6-yl]-oxy}methyl)pyrrolidin-2-one

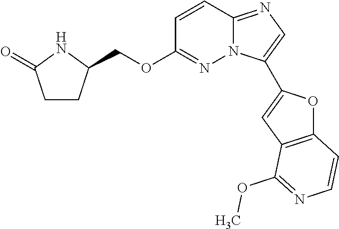 [SMILES: COc1nccc2oc(-c3cnc4ccc(OC[C@H]5CCC(=O)N5)nn34)cc12]

In an ice bath, 7.1 g (61 mmol) (5R)-5-(hydroxymethyl)pyrrolidin-2-one were added to 311 g (61 mmol) sodium hydride (60% in mineral oil) in a mixture of 350 mL anhydrous THF and 200 mL anhydrous DMF. After 15 min of stirring on the ice bath, 10 g (30 mmol) of 6-chloro-3-(4-methoxyfuro[3,2-c]pyridin-2-yl)imidazo[1,2-b]-pyridazine were added. The ice bath was removed and the mixture was stirred for 19 h at 40° C.

The reaction mixture was concentrated in vacuum. The precipitate was filtered off, washed with water and dried in vacuum. The obtained crude material was digested in methanol to yield 9.2 g of the title compound as solid material.

¹H-NMR (500 MHz, DMSO-d₆): δ [ppm]=1.93-2.04 (1H), 2.16-2.25 (1H), 2.25-2.30 (1H), 2.31-2.40 (1H), 4.03-4.12 (4H), 4.41 (1H), 4.51 (1H), 7.07 (1H), 7.38 (1H), 7.47 (1H), 7.92 (1H), 8.07 (1H), 8.18 (1H), 8.19 (1H).

LC-MS (Method 21): Rₜ=0.92 min; MS (ESIpos) m/z=380 [M+H]⁺.

### Example IV-7

### 1-[(2S)-2-(2-{[3-(Furo[3,2-c]pyridin-2-yl)imidazo[1,2-b]pyridazin-6-yl]-oxy}ethyl)pyrrolidin-1-yl]-2,2-dimethylpropan-1-one

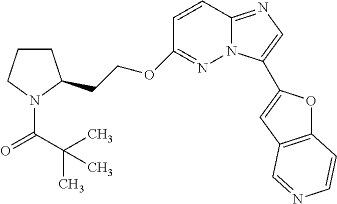 [SMILES: CC(C)(C)C(=O)N1CCC[C@H]1CCOc1ccc2ncc(-c3cc4cnccc4o3)n2n1]

To 80 mg (0.23 mmol) 3-(furo[3,2-c]pyridin-2-yl)-6-{2-[(2S)-pyrrolidin-2-yl]ethoxy}-imidazo[1,2-b]-pyridazine (used as crude product) in 4 mL THF were added 74 μL (0.92 mmol) pyridine and 186 μL (0.92 mmol) 2,2-dimethylpropanoic anhydride. The mixture was stirred a for 3 h at room temperature.

50 μL of water were added and the mixture was stirred for 5 min. Saturated aqueous sodium hydrogen carbonate solution was added and the mixture was extracted with ethyl acetate. The organic layer was dried over sodium sulfate and evaporated. The obtained crude product was purified by flash chromatography followed by HPLC to give 39 mg of the title compound as solid material.

1H-NMR (300 MHz, DMSO-d6), δ [ppm]=1.14 (9H), 1.67-2.01 (5H), 2.13-2.28 (1H), 3.44-3.57 (1H), 3.65 (1H), 4.21-4.33 (1H), 4.49 (2H), 7.01 (1H), 7.66-7.74 (2H), 8.12-8.21 (2H), 8.46 (1H), 8.99 (1H)

LC-MS (Method 21): Rₜ=0.89 min; MS (ESIpos) m/z=434 [M+H]⁺.

### Example IV-8

### Cyclopropyl[(2R)-2-({[3-(4-methoxyfuro[3,2-c]pyridin-2-yl)imidazo[1,2-b]-pyridazin-6-yl]oxy}methyl)morpholin-4-yl]methanone

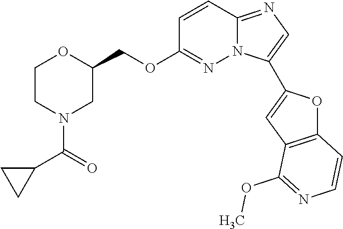 [SMILES: COc1nccc2oc(-c3cnc4ccc(OC[C@H]5CN(C(=O)C6CC6)CCO5)nn34)cc12]

To 150 mg (0.39 mmol) 3-(4-methoxyfuro[3,2-c]pyridin-2-yl)-6-[(2R)-morpholin-2-ylmethoxy]imidazo-[1,2-b]pyridazine in 5 mL dichloromethane were added 64 μL (0.79 mmol) pyridine and 44 μL (0.47 mmol) cyclopropanecarbonyl chloride. The mixture was stirred for 24 h at room temperature.

The mixture was poured into brine and extracted with dichloromethane. The organic layer was dried over sodium sulfate and evaporated. The obtained crude product was purified by HPLC to give 33 mg of the title compound as solid material.

¹H-NMR (400 MHz, DMSO-d₆), δ [ppm]=0.71 (4H), 1.91-2.01 (1H), 2.68-2.87 (1H), 3.38-3.61 (1H), 3.93 (2H), 4.02 (3H), 4.15 (1H), 4.30-4.48 (1H), 4.56 (2H), 7.09 (1H), 7.37 (1H), 7.51 (1H), 8.04 (1H), 8.15-8.21 (2H)

LC-MS (Method 21): Rₜ=1.05 min; MS (ESIpos) m/z=450 [M+H]⁺.

### Example IV-9

### 6-({[3-(4-Methoxyfuro[3,2-c]pyridin-2-yl)imidazo[1,2-b]pyridazin-6-yl]oxy}-methyl)piperidin-2-one

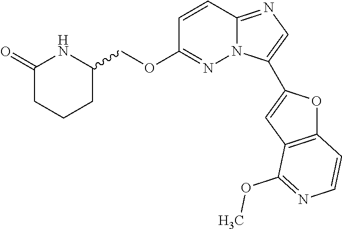 [SMILES: COc1nccc2oc(-c3cnc4ccc(OCC5CCCC(=O)N5)nn34)cc12]

In an ice bath, 85 mg (0.65 mmol) 6-(hydroxymethyl)-piperidin-2-one were added to 26 mg (0.65 mmol) sodium hydride (60% in mineral oil) in a mixture of 2.5 mL anhydrous THF and 0.9 mL anhydrous DMF. After 15 min of stirring on the ice bath, 150 mg (0.32 mmol) of 6-chloro-3-(4-methoxyfuro[3,2-c]pyridin-2-yl)imidazo-[1,2-b]pyridazine were added. The ice bath was removed and the mixture was stirred for 15 h at 40° C.

The reaction mixture was poured into water and extracted with ethyl acetate. The organic layer was dried over magnesium sulfate, and concentrated. The residue was purified by HPLC to yield 20 mg of the title compound as solid material.

¹H-NMR (300 MHz, DMSO-d₆), δ [ppm]=1.57-1.74 (2H), 1.78-2.02 (2H), 2.12-2.21 (2H), 3.82 (1H), 4.01 (3H), 4.44 (2H), 7.06 (1H), 7.36 (1H), 7.46 (1H), 7.61 (1H), 8.04 (1H), 8.13-8.20 (2H).

LC-MS (Method 21): Rₜ=0.97 min; MS (ESIpos) m/z=394 [M+H]⁺.

### Example IV-10

### 2,2,2-Trifluoro-1-[(2R)-2-({[3-(4-methoxyfuro[3,2-c]pyridin-2-yl)imidazo-[1,2-b]pyridazin-6-yl]oxy}methyl)morpholin-4-yl]ethanone

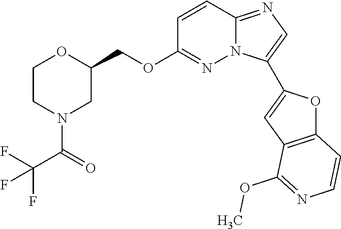 [SMILES: COc1nccc2oc(-c3cnc4ccc(OC[C@H]5CN(C(=O)C(F)(F)F)CCO5)nn34)cc12]

To 150 mg (0.39 mmol) 3-(4-methoxyfuro[3,2-c]pyridin-2-yl)-6-[(2R)-morpholin-2-ylmethoxy]imidazo-[1,2-b]pyridazine in 5 mL dichloromethane were added 127 μL (1.57 mmol) pyridine and 109 μL (0.79 mmol) trifluoro acetic anhydride. The mixture was stirred for 24 h at room temperature.

The mixture was poured into brine and extracted with dichloromethane. The organic layer was dried over sodium sulfate and evaporated. The obtained crude product was purified by HPLC to give 11 mg of the title compound as solid material.

¹H-NMR (400 MHz, DMSO-d₆): δ [ppm]=3.09-3.55 (2H), 3.65 (1H), 3.74-3.92 (1H), 3.98-4.11 (6H), 4.38 (1H), 4.55-4.68 (2H), 7.04 (1H), 7.32 (1H), 7.52 (1H), 8.05 (1H), 8.10-8.16 (2H).

LC-MS (Method 21): Rₜ=1.19 min; MS (ESIpos) m/z=478 [M+H]⁺.

### Example IV-11

### 1-[(2R)-2-({[3-(4-Methoxyfuro[3,2-c]pyridin-2-yl)imidazo[1,2-b]pyridazin-6-yl]oxy}-methyl)morpholin-4-yl]-2,2-dimethylpropan-1-one

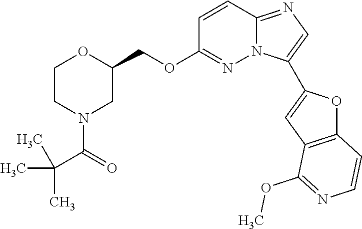 [SMILES: COc1nccc2oc(-c3cnc4ccc(OC[C@H]5CN(C(=O)C(C)(C)C)CCO5)nn34)cc12]

To 150 mg (0.39 mmol) 3-(4-methoxyfuro[3,2-c]pyridin-2-yl)-6-[(2R)-morpholin-2-ylmethoxy]imidazo-[1,2-b]pyridazine in 5 mL dichloromethane were added 64 μL (0.79 mmol) pyridine and 59 μL (0.47 mmol) 2,2-dimethylpropanoyl chloride. The mixture was stirred for 24 h at room temperature.

The mixture was poured into brine and extracted with dichloromethane. The organic layer was dried over sodium sulfate and evaporated. The obtained crude product was purified by HPLC to give 18 mg of the title compound as solid material.

¹H-NMR (400 MHz, DMSO-d₆), δ [ppm]=1.17 (9H), 2.92-3.08 (2H), 3.48 (1H), 3.81-3.87 (1H), 3.89 (1H), 4.02 (3H), 4.09-4.17 (1H), 4.29-4.39 (1H), 4.47-4.61 (2H), 7.07 (1H), 7.37 (1H), 7.51 (1H), 8.04 (1H), 8.14-8.21 (2H).

LC-MS (Method 21): Rₜ=1.18 min; MS (ESIpos) m/z=466 [M+H]⁺.

### Example IV-12

### (5R)-5-[({3-[4-(Cyclopropylmethoxy)furo[3,2-c]pyridin-2-yl]imidazo[1,2-b]-pyridazin-6-yl}oxy)methyl]pyrrolidin-2-one

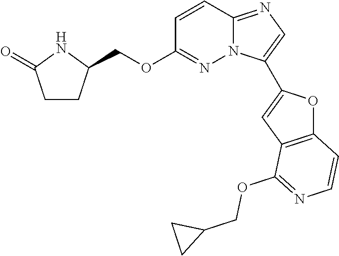 [SMILES: O=C1CC[C@H](COc2ccc3ncc(-c4cc5c(OCC6CC6)nccc5o4)n3n2)N1]

In an ice bath, 21 mg (0.54 mmol) sodium hydride (60% in mineral oil) were added to 63 mg (0.54 mmol) (5R)-5-hydroxymethyl)-pyrrolidin-2-one in 1 mL anhydrous DMF and 3 mL anhydrous THF. After 15 min of stirring on the ice bath, 120 mg (0.27 mmol) of 6-chloro-3-[4-(cyclopropylmethoxy)furo[3,2-c]pyridin-2-yl]imidazo[1,2-b]-pyridazine were added. The ice bath was removed and the mixture was stirred for 18 h at 40° C.

The reaction mixture was poured into water and extracted with ethyl acetate. The crude product was taken up in a mixture of DMSO and DMF. Insoluble material was filtered off. The title compound precipitated upon standing over night. The precipitate was filtered off and dried in vacuum to give 34 mg of the title compound as solid material.

¹H-NMR (500 MHz, DMSO-d₆): δ [ppm]=0.38-0.43 (2H), 0.57-0.63 (2H), 1.30-1.41 (1H), 1.89-2.00 (1H), 2.16-2.25 (1H), 2.27-2.39 (2H), 4.10-4.17 (1H), 4.30-4.35 (2H), 4.40 (1H), 4.56 (1H), 7.07 (1H), 7.36 (1H), 7.51 (1H), 7.95 (1H), 8.03 (1H), 8.19 (1H), 8.21 (1H).

LCMS (Method 21): Rₜ=1.10 min; MS (ESIpos) m/z=420 [M+H]⁺.

### Example IV-13

### (6R)-6-[({3-[4-(Propan-2-yloxy)furo[3,2-c]pyridin-2-yl]imidazo[1,2-b]pyridazin-6-yl}oxy)methyl]piperazin-2-one

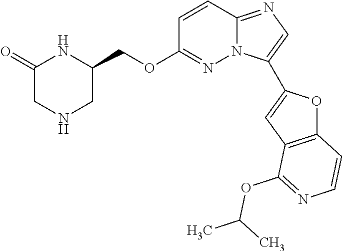 [SMILES: CC(C)Oc1nccc2oc(-c3cnc4ccc(OC[C@H]5CNCC(=O)N5)nn34)cc12]

In an ice bath, 210 mg (0.91 mmol) (6R)-4-(2,2-dimethylpropanoyl)-6-(hydroxy-methyl)piperazin-2-one in 2 mL anhydrous THF were added to 41 mg (1.0 mmol) sodium hydride (60% in mineral oil) in 2 mL anhydrous THF. After 15 min of stirring on the ice bath, 170 mg (0.52 mmol) of 6-chloro-3-[4-(propan-2-yloxy)furo[3,2-c]pyridin-2-yl]imidazo[1,2-b]pyridazine were added. The ice bath was removed and the mixture was stirred for 16 h at 40° C.

Brine was added, and the mixture was extracted with ethyl acetate. The organic layer was dried over magnesium sulfate, and concentrated.

6 mL dichloromethane were added to the obtained crude product. The mixture was treated with 330 μL (2.6 mmol) TFA and stirred for 24 h at room temperature. Again, 4 mL of dichloromethane and 1 mL of methanol were added and stirring at room temperature was continued for 6 h. Another 100 μL TFA were added and the mixture was stirred for 48 h at room temperature. Once more, 2 mL TFA were added and stirring at room temperature was continued for 4 h.

Saturated aqueous sodium hydrogen carbonate solution was added and the mixture was extracted with ethyl acetate. The organic layer was dried over sodium sulfate and evaporated. The crude product was purified by HPLC to yield 41 mg of the title compound as solid material.

¹H-NMR (400 MHz, DMSO-d₆): δ [ppm]=1.40 (9H), 3.07 (1H), 3.25 (2H), 3.40 (2H), 3.81 (1H), 3.95 (1H), 4.52 (1H), 4.59-4.66 (1H), 5.47 (1H), 7.10 (1H), 7.34 (1H), 7.49 (1H), 8.05 (1H), 8.11 (1H), 8.19 (1H), 8.22 (1H).

LC-MS (Method 21): Rₜ=0.77 min; MS (ESIpos) m/z=423 [M+H]⁺.

### Example IV-14

### (5R)-5-[({3-[4-(Ethylamino)furo[3,2-c]pyridin-2-yl]imidazo[1,2-b]pyridazin-6-yl}oxy)methyl]pyrrolidin-2-one

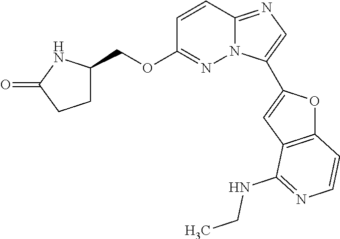 [SMILES: CCNc1nccc2oc(-c3cnc4ccc(OC[C@H]5CCC(=O)N5)nn34)cc12]

To a stirred suspension of tert-butyl ethyl[2-(6-{[(2R)-5-oxopyrrolidin-2-yl]-methoxy}imidazo[1,2-b]pyridazin-3-yl)furo[3,2-c]pyridin-4-yl]carbamate (125 mg) in dichloromethane (1 mL) was added TFA (0.4 mL). The mixture was stirred at room temperature for 4 h. The solvent was removed in vacuum. The residue was dissolved in dichloromethane and methanol, and a saturated solution of potassium carbonate was added until pH 9 was reached. The mixture was stirred for 30 minutes. The organic phase was separated and dried (sodium sulfate) and the solvent was removed in vacuum. Silicagel chromatography gave 75 mg of the title compound as a solid.

¹H-NMR (300 MHz, DMSO-d₆), δ [ppm]=1.20 (3H), 1.91-2.02 (1H), 2.07-2.36 (3H), 3.41-3.54 (2H), 4.00-4.11 (1H), 4.40-4.57 (2H), 6.83 (1H), 7.00 (1H), 7.13 (1H), 7.67 (1H), 7.87 (1H), 7.94 (1H), 8.07 (1H), 8.14 (1H).

LCMS (Method 20): Rₜ=0.88 min; MS (ESIpos) m/z=393 [M+H]⁺.

Further, the compounds of formula (I) of the present invention can be converted to any salt as described herein, by any method which is known to the person skilled in the art. Similarly, any salt of a compound of formula (I) of the present invention can be converted into the free compound, by any method which is known to the person skilled in the art.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃)
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate)
flavourants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, crosslinked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide); tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

### Sterile IV Solution:

A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

### Lyophilised Powder for IV Administration:

A sterile preparation can be prepared with (i) 100-1000 mg of the desired compound of this invention as a lyophilised powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

### Intramuscular Suspension:

The following solution or suspension can be prepared, for intramuscular injection:

50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol

### Hard Shell Capsules:

A large number of unit capsules are prepared by filling standard two-piece hard galantine capsules each with 100 mg of powdered active ingredient, 150 mg of lactose, 50 mg of cellulose and 6 mg of magnesium stearate.

### Soft Gelatin Capsules:

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

### Tablets:

A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

### Immediate Release Tablets/Capsules:

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Combination Therapies

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

In accordance with an embodiment, the present invention relates to pharmaceutical combinations comprising:
one or more first active ingredients selected from a compound of general formula (I) as defined supra, and
one or more second active ingredients selected from chemotherapeutic anti-cancer agents.

The term “chemotherapeutic anti-cancer agents”, includes but is not limited to:

131l-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 1000394, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, copanlisib, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin, or a combination thereof.

The additional pharmaceutical agent can be afinitor, aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BAY 80-6946, BCG or tice BCG, bestatin, betamethasone acetate, betamethasone sodium phosphate, bexarotene, bleomycin sulfate, broxuridine, bortezomib, busulfan, calcitonin, campath, capecitabine, carboplatin, casodex, cefesone, celmoleukin, cerubidine, chlorambucil, cisplatin, cladribine, clodronic acid, cyclophosphamide, cytarabine, dacarbazine, dactinomycin, DaunoXome, decadron, decadron phosphate, delestrogen, denileukin diftitox, depo-medrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine phosphate sodium, ethinyl estradiol, ethyol, etidronic acid, etopophos, etoposide, fadrozole, farston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron HCl, histrelin, hycamtin, hydrocortone, eyrthro-hydroxynonyladenine, hydroxyurea, ibritumomab tiuxetan, idarubicin, ifosfamide, interferon alpha, interferon-alpha 2, interferon alfa-2A, interferon alfa-2B, interferon alfa-nl, interferon alfa-n3, interferon beta, interferon gamma-la, interleukin-2, intron A, iressa, irinotecan, kytril, lapatinib, lentinan sulfate, letrozole, leucovorin, leuprolide, leuprolide acetate, levamisole, levofolinic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, Mesna, methotrexate, metvix, miltefosine, minocycline, mitomycing C, mitotane, mitoxantrone, Modrenal, Myocet, nedaplatin, neulasta, neumega, neupogen, nilutamide, nolvadex, NSC-631570, OCT-43, octreotide, ondansetron HCl, orapred, oxaliplatin, paclitaxel, pediapred, pegaspargase, Pegasys, pentostatin, picibanil, pilocarpine HCl, pirarubicin, plicamycin, porfimer sodium, prednimustine, prednisolone, prednisone, premarin, procarbazine, procrit, raltitrexed, RDEA 119, rebif, rhenium-186 etidronate, rituximab, roferon-A, romurtide, salagen, sandostatin, sargramostim, semustine, sizofiran, sobuzoxane, solu-medrol, sparfosic acid, stem-cell therapy, streptozocin, strontium-89 chloride, sunitinib, synthroid, tamoxifen, tamsulosin, tasonermin, tastolactone, taxotere, teceleukin, temozolomide, teniposide, testosterone propionate, testred, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran, ABI-007, acolbifene, actimmune, affinitak, aminopterin, arzoxifene, asoprisnil, atamestane, atrasentan, sorafenib (BAY 43-9006), avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole limpet hemocyanin, L-651582, lanreotide, lasofoxifene, libra, lonafarnib, miproxifene, minodronate, MS-209, liposomal MTP-PE, MX-6, nafarelin, nemorubicin, neovastat, nolatrexed, oblimersen, onco-TCS, osidem, paclitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thymosin alpha 1, tiazofurine, tipifarnib, tirapazamine, TLK-286, toremifene, TransMID-107R, valspodar, vapreotide, vatalanib, verteporfin, vinflunine, Z-100, zoledronic acid or combinations thereof.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, epothilone, an epothilone derivative, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifene, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone and its derivatives, irinotecan, raloxifene and topotecan.

The compounds of the invention may also be administered in combination with protein therapeutics. Such protein therapeutics suitable for the treatment of cancer or other angiogenic disorders and for use with the compositions of the invention include, but are not limited to, an interferon (e.g., interferon .alpha., .beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, denileukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC10, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, labetuzumab, alpha-particle-emitting radioisotope-Ilinked lintuzumab, EM-1421, HyperAcute vaccine, tucotuzumab celmoleukin, galiximab, HPV-16-E7, Javelin—prostate cancer, Javelin—melanoma, NY-ESO-1 vaccine, EGF vaccine, CYT-004-MelQbG10, WT1 peptide, oregovomab, ofatumumab, zalutumumab, cintredekin besudotox, WX-G250, Albuferon, aflibercept, denosumab, vaccine, CTP-37, efungumab, or 131l-chTNT-1/B. Monoclonal antibodies useful as the protein therapeutic include, but are not limited to, muromonab-CD3, abciximab, edrecolomab, daclizumab, gentuzumab, alemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

The compounds of the invention may also be combined with biological therapeutic agents, such as antibodies (e.g. avastin, rituxan, erbitux, herceptin), or recombinant proteins.

In accordance with an embodiment, the present invention relates to pharmaceutical combinations comprising:
one or more compounds of general formula (I), supra, or a stereoisomer, a tautomer, an N-oxide, a hydrate, a solvate, or a salt thereof, particularly a pharmaceutically acceptable salt thereof, or a mixture of same;
   and
one or more agents selected from: a taxane, such as Docetaxel, Paclitaxel, lapatinib, sunitinib, or Taxol; an epothilone, such as Ixabepilone, Patupilone, or Sagopilone; Mitoxantrone; Predinisolone; Dexamethasone; Estramustin; Vinblastin; Vincristin; Doxorubicin; Adriamycin; Idarubicin; Daunorubicin; Bleomycin; Etoposide; Cyclophosphamide; Ifosfamide; Procarbazine; Melphalan; 5-Fluorouracil; Capecitabine; Fludarabine; Cytarabine; Ara-C; 2-Chloro-2′-deoxyadenosine; Thioguanine; an anti-androgen, such as Flutamide, Cyproterone acetate, or Bicalutamide; Bortezomib; a platinum derivative, such as Cisplatin, or Carboplatin; Chlorambucil; Methotrexate; and Rituximab.

The compounds of the invention may also be in combination with antiangiogenesis agents, such as, for example, with avastin, axitinib, DAST, recentin, sorafenib or sunitinib. Combinations with inhibitors of proteasomes or mTOR inhibitors, or anti-hormones or steroidal metabolic enzyme inhibitors are also possible.

Generally, the use of cytotoxic and/or cytostatic agents in combination with a compound or composition of the present invention will serve to:

(1) yield better efficacy in reducing the growth of a tumour or even eliminate the tumour as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumour progression, and/or
(8) yield efficacy and tolerability results at least as good as those of the agents used alone, compared to known instances where other cancer agent combinations produce antagonistic effects.

### Methods of Sensitizing Cells to Radiation

In a distinct embodiment of the present invention, a compound of the present invention may be used to sensitize a cell to radiation. That is, treatment of a cell with a compound of the present invention prior to radiation treatment of the cell renders the cell more susceptible to DNA damage and cell death than the cell would be in the absence of any treatment with a compound of the invention. In one aspect, the cell is treated with at least one compound of the invention.

Thus, the present invention also provides a method of killing a cell, wherein a cell is administered one or more compounds of the invention in combination with conventional radiation therapy.

The present invention also provides a method of rendering a cell more susceptible to cell death, wherein the cell is treated with one or more compounds of the invention prior to the treatment of the cell to cause or induce cell death. In one aspect, after the cell is treated with one or more compounds of the invention, the cell is treated with at least one compound, or at least one method, or a combination thereof, in order to cause DNA damage for the purpose of inhibiting the function of the normal cell or killing the cell.

In one embodiment, a cell is killed by treating the cell with at least one DNA damaging agent. That is, after treating a cell with one or more compounds of the invention to sensitize the cell to cell death, the cell is treated with at least one DNA damaging agent to kill the cell. DNA damaging agents useful in the present invention include, but are not limited to, chemotherapeutic agents (e.g., cisplatinum), ionizing radiation (X-rays, ultraviolet radiation), carcinogenic agents, and mutagenic agents.

In another embodiment, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of the invention is administered to a cell prior to the radiation or other induction of DNA damage in the cell. In another aspect of the invention, a compound of the invention is administered to a cell concomitantly with the radiation or other induction of DNA damage in the cell. In yet another aspect of the invention, a compound of the invention is administered to a cell immediately after radiation or other induction of DNA damage in the cell has begun.

In another aspect, the cell is in vitro. In another embodiment, the cell is in vivo.

As mentioned supra, the compounds of the present invention have surprisingly been found to effectively inhibit MKNK-1 and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In accordance with another aspect therefore, the present invention covers a compound of general formula (I), or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula (I) described supra for manufacturing a pharmaceutical composition for the treatment or prophylaxis of a disease.

The diseases referred to in the two preceding paragraphs are diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Preferably, the use is in the treatment or prophylaxis of diseases, wherein the diseases are haemotological tumours, solid tumours and/or metastases thereof.

### Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant tyrosine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

### Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

### Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases of said method are haematological tumours, solid tumour and/or metastases thereof.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The example testing experiments described herein serve to illustrate the present invention and the invention is not limited to the examples given.

### Biological Assays:

Examples were tested in selected biological assays one or more times. When tested more than once, data are reported as either average values or as median values, wherein:
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

### MKNK1 Kinase Assay

MKNK1-inhibitory activity of compounds of the present invention was quantified employing the MKNK1 TR-FRET assay as described in the following paragraphs.

A recombinant fusion protein of Glutathione-S-Transferase (GST, N-terminally) and human full-lengt MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of MKNK1 in aqueous assay buffer [50 mM HEPES pH 7.5, 5 mM magnesium chloride, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (0.1 μM=>final conc. in the 5 μL assay volume is 0.06 μM) in assay buffer and the resulting mixture was incubated for a reaction time of 45 min at 22° C. The concentration of MKNK1 was adjusted depending of the activity of the enzyme lot and was chosen appropriate to have the assay in the linear range, typical concentrations were in the range of 0.05 ag/ml. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values were calculated by a 4 parameter fit.

TABLE 1a
MKNK1 IC₅₀s

| Example | MKNK1 IC50 [nM] |
|---|---|
| I-1 | 1.0 |
| I-2 | 1.8 |
| I-3 | 1.2 |
| I-4 | 5.6 |
| I-5 | 5.3 |
| I-6 | 3.4 |
| I-7 | 3.1 |
| I-8 | 5.2 |
| I-9 | 3.3 |
| I-10 | 3.2 |
| I-11 | 2.9 |
| I-12 | 3.5 |
| I-13 | 3.7 |
| I-14 | 5.1 |
| I-15 | 5.6 |
| I-16 | 4.7 |
| I-17 | 4.6 |
| I-18 | 7.1 |
| I-19 | 4.5 |
| I-20 | 7.7 |
| I-21 | 6.6 |
| I-22 | 12.6 |
| I-23 | 15.7 |
| I-24 | 12.5 |
| I-25 | 4.7 |
| I-26 | 9.0 |
| I-27 | 7.3 |
| I-28 | 9.1 |
| I-29 | 13.1 |
| I-30 | 10.3 |
| I-31 | 16.7 |
| I-32 | 13.7 |
| I-33 | 15.5 |
| I-34 | 16.4 |
| I-35 | 17.9 |
| I-36 | 15.1 |
| I-37 | 17.0 |
| I-38 | 64.0 |
| I-39 | 55.2 |
| I-40 | 26.0 |
| I-41 | |
| I-42 | 14.2 |
| I-43 | 1.0 |
| I-44 | 1.1 |
| I-45 | 1.3 |
| I-46 | 1.2 |
| I-47 | 1.6 |
| I-48 | 1.8 |
| I-49 | 1.1 |
| I-50 | 2.4 |
| I-51 | 2.1 |
| I-52 | 1.7 |
| I-53 | 2.4 |
| I-54 | 2.0 |
| I-55 | 2.1 |
| I-56 | 4.0 |
| I-57 | 1.9 |
| I-58 | 2.6 |
| I-59 | 2.8 |
| I-60 | 2.2 |
| I-61 | 2.9 |
| I-62 | 3.5 |
| I-63 | 4.0 |
| I-64 | 4.7 |
| I-65 | 5.2 |
| I-66 | 7.3 |
| I-67 | 6.6 |
| I-68 | 7.8 |
| I-69 | 13.7 |
| I-70 | 15.1 |
| I-71 | 4.6 |
| I-72 | 4.8 |
| I-73 | 5.6 |
| I-74 | 5.4 |
| I-75 | 6.0 |
| I-76 | 7.3 |
| I-77 | 7.9 |
| I-78 | 6.7 |
| I-79 | 10.1 |
| I-80 | 24.5 |

TABLE 1b
MKNK1 IC₅₀s

| Example | MKNK1 IC₅₀ [nM] |
|---|---|
| II-1 | 8.5 |
| II-2 | 16 |
| II-3 | 110 |
| II-4 | 18 |
| II-5 | 15 |
| II-6 | 7.1 |
| II-7 | 6.3 |
| II-8 | 2.0 |
| II-9 | 0.8 |
| II-10 | 6.5 |
| II-11 | 2.4 |
| II-12 | 24 |
| II-13 | 39 |
| II-14 | 7.0 |
| II-15 | 59 |
| II-16 | 13 |
| II-17 | 0.6 |
| II-18 | 0.4 |
| II-19 | 0.4 |
| II-20 | 0.5 |
| II-21 | 0.3 |
| II-22 | 371 |
| II-23 | 2.0 |
| II-24 | 3.3 |
| II-25 | 2.4 |
| II-26 | 0.8 |
| II-27 | 2.2 |
| II-28 | 5.1 |
| II-29 | 5.4 |
| II-30 | 2.3 |
| II-31 | 24.2 |
| II-32 | 4.4 |
| II-33 | 16.3 |
| II-34 | 26.7 |
| II-35 | 16.7 |
| II-36 | 2.3 |
| II-37 | 43.9 |
| II-38 | 4.6 |
| II-39 | 1.6 |
| II-43 | 21.7 |
| II-44 | 2.9 |
| II-45 | 5.7 |
| II-46 | 1.4 |
| II-47 | 4.8 |
| II-48 | 7.0 |
| II-49 | 5.2 |
| II-50 | 39.1 |
| II-51 | 2.8 |
| II-53 | 1.1 |
| II-54 | 2.3 |
| II-55 | 3.5 |
| II-56 | 5.4 |
| II-57 | 53.7 |
| II-58 | 2.6 |
| II-59 | 6.4 |
| II-61 | 23.6 |
| II-62 | 5.1 |
| II-63 | 15.2 |
| II-64 | 9.3 |
| II-65 | 0.9 |
| II-66 | 27.3 |
| II-67 | 23.5 |
| II-68 | 23.2 |
| II-69 | 29.4 |
| II-70 | 41.4 |
| II-71 | 6.5 |
| II-72 | 15.7 |
| II-73 | 42.4 |
| II-74 | 4.1 |
| II-75 | 23.0 |
| II-76 | 6.9 |

TABLE 1c
MKNK1 IC₅₀s

| Example | MKNK1 IC₅₀ [nM] |
|---|---|
| III-1 | 8.4 |
| III-2 | 1.8 |
| III-3 | 6.7 |
| III-4 | 0.6 |
| III-7 | 11.6 |
| III-8 | 7.2 |
| III-9 | 10.6 |
| III-10 | 15.4 |
| III-11 | 2.7 |
| III-12 | 8.6 |
| III-13 | 13.0 |
| III-14 | 6.9 |
| III-15 | 31.0 |
| III-16 | 2.1 |
| III-17 | 18.3 |
| III-18 | 0.7 |
| III-19 | 12.2 |
| III-20 | 3.9 |
| III-21 | 12.0 |
| III-22 | 6.5 |
| III-23 | 16.6 |
| III-24 | 0.4 |
| III-25 | 9.2 |
| III-26 | 1.7 |
| III-27 | 31.1 |
| III-30 | 5.0 |
| III-31 | 0.5 |
| III-32 | 0.6 |
| III-33 | 1.1 |
| III-34 | 3.5 |
| III-35 | 1.0 |
| III-36 | 11.6 |
| III-37 | 1.1 |
| III-38 | 0.5 |
| III-39 | 0.9 |
| III-40 | 1.5 |
| III-41 | 12.0 |
| III-42 | 2.2 |
| III-43 | 5.7 |
| III-44 | 5.5 |
| III-45 | 4.4 |
| III-46 | 3.9 |
| III-47 | 13.6 |
| III-48 | 11.9 |
| III-49 | 21.3 |
| III-50 | 1.4 |
| III-51 | 7.3 |
| III-52 | 1.8 |
| III-53 | 8.7 |
| III-54 | 2.8 |
| III-55 | 3.7 |
| III-56 | 20.8 |
| III-57 | 1.5 |
| III-58 | 1.0 |
| III-61 | 1.0 |
| III-62 | 2.4 |
| III-63 | 4.3 |
| III-64 | 0.4 |
| III-65 | 11.6 |
| III-66 | 4.2 |
| III-67 | 10.7 |
| III-68 | 0.5 |
| III-69 | 0.4 |
| III-70 | 4.8 |
| III-71 | 2.0 |
| III-72 | 1.0 |
| III-73 | 0.6 |
| III-74 | 1.0 |
| III-75 | 0.8 |
| III-76 | 3.5 |
| III-77 | 0.7 |
| III-78 | 0.2 |
| III-80 | 0.4 |
| III-81 | 0.3 |
| III-82 | 0.4 |
| III-83 | 11.6 |
| III-85 | 4.8 |
| III-86 | 0.7 |
| III-89 | 2.9 |
| III-90 | 0.8 |
| III-91 | 8.4 |
| III-95 | 3.2 |
| III-96 | 3.9 |
| III-97 | 5.6 |
| III-98 | 1.6 |
| III-99 | 24.4 |
| III-100 | 5.7 |
| III-101 | 0.8 |
| III-102 | 6.8 |
| III-103 | 9.1 |
| III-104 | 18.7 |
| III-105 | 2.1 |
| III-106 | 10.2 |
| III-107 | 4.8 |
| III-108 | 1.9 |
| III-111 | 3.8 |
| III-112 | 15.6 |
| III-113 | 0.5 |
| III-114 | 10.4 |
| III-115 | 18.6 |
| III-116 | 27.0 |
| III-117 | 21.5 |
| III-118 | 2.5 |
| III-119 | 8.8 |
| III-120 | 11.2 |
| III-121 | 11.6 |
| III-122 | 12.3 |
| III-123 | 2.2 |
| III-124 | 16.8 |
| III-125 | 7.4 |
| III-126 | 5.3 |
| III-128 | 12.8 |
| III-129 | 16.8 |
| III-130 | 5.3 |
| III-131 | 11.9 |
| III-132 | 6.8 |
| III-133 | 2.7 |
| III-134 | 2.0 |
| III-135 | 30.1 |
| III-136 | 52.5 |
| III-137 | 2.7 |
| III-139 | 3.0 |
| III-140 | 80.0 |
| III-141 | 3.3 |
| III-142 | 1.6 |
| III-143 | 3.7 |
| III-144 | 4.6 |
| III-145 | 9.7 |
| III-146 | 27.0 |

TABLE 1d
MKNK1 IC₅₀s

| Example | MKNK1 IC₅₀ [nM] |
|---|---|
| IV-1 | 5 |
| IV-2 | 11 |
| IV-3 | 10 |
| IV-4 | 2 |
| IV-5 | 2 |
| IV-6 | 4 |
| IV-7 | 1 |
| IV-8 | 4 |
| IV-9 | 3 |
| IV-10 | 7 |
| IV-11 | 6 |
| IV-13 | 22 |
| IV-14 | 1 |

### MKNK1 Kinase High ATP Assay

MKNK1-inhibitory activity at high ATP of compounds of the present invention after their preincubation with MKNK1 was quantified employing the TR-FRET-based MKNK1 high ATP assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used, which can be purchased e.g. from the company Biosyntan (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of MKNK1 in aqueous assay buffer [50 mM HEPES pH 7.5, 5 mM magnesium chloride, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 3.3 mM=>final conc. in the 5 μL assay volume is 2 mM) and substrate (0.1 μM=>final conc. in the 5 μL assay volume is 0.06 μM) in assay buffer and the resulting mixture was incubated for a reaction time of 30 min at 22° C. The concentration of MKNK1 was adjusted depending of the activity of the enzyme lot and was chosen appropriate to have the assay in the linear range, typical concentrations were in the range of 0.003 μg/mL. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values were calculated by a 4 parameter fit.

TABLE 2a
MKNK1 high ATP IC₅₀s

| | MKNK1 high |
|---|---|
| Example | ATP IC₅₀ [nM] |
| I-1 | 2.3 |
| I-2 | 3.1 |
| I-3 | 4.8 |
| I-4 | 8.8 |
| I-5 | 8.9 |
| I-6 | 9.2 |
| I-7 | 9.2 |
| I-8 | 9.3 |
| I-9 | 9.6 |
| I-10 | 9.9 |
| I-11 | 10.0 |
| I-12 | 10.2 |
| I-13 | 10.7 |
| I-14 | 10.9 |
| I-15 | 12.5 |
| I-16 | 13.3 |
| I-17 | 13.7 |
| I-18 | 13.8 |
| I-19 | 15.4 |
| I-20 | 15.5 |
| I-21 | 17.7 |
| I-22 | 18.6 |
| I-23 | 18.8 |
| I-24 | 20.7 |
| I-25 | 20.8 |
| I-26 | 23.0 |
| I-27 | 23.5 |
| I-28 | 29.8 |
| I-29 | 34.1 |
| I-30 | 34.8 |
| I-31 | 35.2 |
| I-32 | 39.8 |
| I-33 | 40.5 |
| I-34 | 45.1 |
| I-35 | 47.8 |
| I-36 | 56.2 |
| I-37 | 70.0 |
| I-38 | 71.7 |
| I-39 | 77.7 |
| I-40 | 76.4 |
| I-41 | 7.0 |
| I-42 | 26.8 |
| I-43 | 1.3 |
| I-44 | 1.7 |
| I-45 | 1.8 |
| I-46 | 1.9 |
| I-47 | 2.2 |
| I-48 | 2.3 |
| I-49 | 2.4 |
| I-50 | 3.2 |
| I-51 | 3.2 |
| I-52 | 3.4 |
| I-53 | 3.5 |
| I-54 | 3.7 |
| I-55 | 3.8 |
| I-56 | 3.8 |
| I-57 | 3.9 |
| I-58 | 5.2 |
| I-59 | 5.3 |
| I-60 | 5.4 |
| I-61 | 6.7 |
| I-62 | 6.8 |
| I-63 | 10.1 |
| I-64 | 10.5 |
| I-65 | 11.8 |
| I-66 | 14.9 |
| I-67 | 20.3 |
| I-68 | 20.6 |
| I-69 | 36.9 |
| I-70 | 45.8 |
| I-71 | 14.4 |
| I-72 | 14.7 |
| I-73 | 16.5 |
| I-74 | 16.7 |
| I-75 | 17.7 |
| I-76 | 21.1 |
| I-77 | 22.2 |
| I-78 | 23.0 |
| I-79 | 25.5 |
| I-80 | 47.7 |

TABLE 2b
MKNK1 high ATP IC₅₀s

| | MKNK1 high |
|---|---|
| Example | ATP IC₅₀ [nM] |
| II-1 | 20 |
| II-2 | 36 |
| II-3 | 196 |
| II-4 | 28 |
| II-5 | 27 |
| II-6 | 14 |
| II-7 | 4.2 |
| II-8 | 2.7 |
| II-9 | 0.6 |
| II-10 | 5.0 |
| II-11 | 3.5 |
| II-12 | 67 |
| II-13 | 58 |
| II-14 | 27 |
| II-15 | 137 |
| II-16 | 15 |
| II-17 | 0.5 |
| II-18 | 0.4 |
| II-19 | 0.4 |
| II-20 | 0.8 |
| II-21 | 0.3 |
| II-22 | 422 |
| II-23 | 1.9 |
| II-24 | 3.2 |
| II-25 | 2.7 |
| II-26 | 0.8 |
| II-27 | 5.7 |
| II-28 | 0.7 |
| II-29 | 2.7 |
| II-30 | 0.6 |
| II-31 | 35.5 |
| II-32 | 8.1 |
| II-33 | 24.4 |
| II-34 | 43.2 |
| II-35 | 49.6 |
| II-36 | 1.0 |
| II-37 | 69.1 |
| II-38 | 1.4 |
| II-39 | 1.1 |
| II-40 | 4.4 |
| II-41 | 5.5 |
| II-42 | 5.8 |
| II-43 | 37.5 |
| II-44 | 3.8 |
| II-45 | 5.9 |
| II-46 | 0.2 |
| II-47 | 0.8 |
| II-48 | 3.9 |
| II-49 | 11.9 |
| II-50 | 50.6 |
| II-51 | 50.8 |
| II-52 | 7.1 |
| II-53 | 1.1 |
| II-54 | 2.4 |
| II-55 | 1.4 |
| II-56 | 3.1 |
| II-57 | 75.1 |
| II-58 | 1.4 |
| II-59 | 2.0 |
| II-60 | 0.3 |
| II-61 | 66.2 |
| II-63 | 39.5 |
| II-65 | 1.3 |
| II-67 | 56.3 |
| II-68 | 30.4 |
| II-69 | 34.8 |
| II-70 | 49.2 |
| II-71 | 6.1 |
| II-72 | 33.0 |
| II-73 | 47.6 |
| II-74 | 11.2 |
| II-75 | 25.7 |
| II-76 | 18.4 |

TABLE 2c
MKNK1 high ATP IC₅₀s

| | MKNK1 high |
|---|---|
| Example | ATP IC₅₀ [nM] |
| III-1 | 19.5 |
| III-2 | 1.8 |
| III-3 | 11.7 |
| III-4 | 0.4 |
| III-5 | 10.1 |
| III-6 | 8.8 |
| III-7 | 21.7 |
| III-8 | 16.3 |
| III-9 | 20.4 |
| III-10 | 63.5 |
| III-11 | 12.4 |
| III-12 | 43.1 |
| III-13 | 62.0 |
| III-14 | 17.9 |
| III-15 | 78.4 |
| III-16 | 6.0 |
| III-17 | 44.2 |
| III-18 | 0.9 |
| III-19 | 42.7 |
| III-20 | 10.5 |
| III-21 | 33.2 |
| III-22 | 14.1 |
| III-23 | 45.0 |
| III-24 | 8.6 |
| III-25 | 13.7 |
| III-26 | 0.8 |
| III-27 | 97.5 |
| III-28 | 9.4 |
| III-29 | 4.8 |
| III-30 | 10.4 |
| III-31 | 3.0 |
| III-32 | 1.5 |
| III-33 | 1.3 |
| III-34 | 7.4 |
| III-35 | 0.9 |
| III-36 | 37.3 |
| III-37 | 11.1 |
| III-38 | 4.8 |
| III-39 | 0.5 |
| III-40 | 2.6 |
| III-41 | 17.6 |
| III-42 | 4.7 |
| III-43 | 5.3 |
| III-44 | 14.9 |
| III-45 | 8.1 |
| III-46 | 3.6 |
| III-47 | 21.2 |
| III-48 | 32.1 |
| III-49 | 38.5 |
| III-50 | 0.5 |
| III-51 | 14.2 |
| III-52 | 3.7 |
| III-53 | 26.7 |
| III-54 | 6.2 |
| III-55 | 7.8 |
| III-56 | 16.9 |
| III-57 | 3.6 |
| III-58 | 4.4 |
| III-59 | 4.9 |
| III-60 | 2.8 |
| III-61 | 1.2 |
| III-62 | 1.0 |
| III-63 | 12.6 |
| III-64 | 0.1 |
| III-65 | 19.9 |
| III-66 | 8.5 |
| III-67 | 11.0 |
| III-68 | 0.7 |
| III-69 | 0.1 |
| III-70 | 13.5 |
| III-71 | 1.4 |
| III-72 | 2.0 |
| III-73 | 0.8 |
| III-74 | 1.5 |
| III-75 | 1.7 |
| III-76 | 6.0 |
| III-77 | 1.1 |
| III-78 | 1.2 |
| III-79 | 0.7 |
| III-80 | 0.2 |
| III-81 | 0.5 |
| III-82 | 0.3 |
| III-83 | 19.1 |
| III-84 | 0.5 |
| III-85 | 5.2 |
| III-86 | 0.5 |
| III-87 | 3.7 |
| III-88 | 4.2 |
| III-89 | 8.0 |
| III-90 | 0.5 |
| III-91 | 18.3 |
| III-92 | 3.2 |
| III-93 | 5.6 |
| III-94 | 4.0 |
| III-95 | 7.8 |
| III-96 | 3.4 |
| III-97 | 21.0 |
| III-98 | 3.3 |
| III-99 | 45.6 |
| III-100 | 45.8 |
| III-101 | 1.2 |
| III-102 | 31.2 |
| III-103 | 18.6 |
| III-104 | 61.5 |
| III-105 | 7.3 |
| III-106 | 9.2 |
| III-107 | 11.8 |
| III-108 | 0.5 |
| III-109 | 5.2 |
| III-110 | 10.4 |
| III-111 | 42.2 |
| III-112 | 88.1 |
| III-113 | 0.3 |
| III-114 | 23.4 |
| III-115 | 84.1 |
| III-116 | 113.9 |
| III-117 | 39.9 |
| III-118 | 1.0 |
| III-119 | 17.0 |
| III-120 | 7.4 |
| III-121 | 12.8 |
| III-122 | 13.0 |
| III-123 | 0.5 |
| III-124 | 13.9 |
| III-125 | 7.1 |
| III-126 | 2.6 |
| III-127 | 4.2 |
| III-128 | 11.8 |
| III-129 | 14.1 |
| III-130 | 1.5 |
| III-131 | 7.0 |
| III-132 | 2.5 |
| III-133 | 13.1 |
| III-134 | 1.0 |
| III-135 | 12.9 |
| III-136 | 121.9 |
| III-137 | 0.6 |
| III-139 | 1.7 |
| III-140 | 183.3 |
| III-141 | 1.4 |
| III-142 | 0.5 |
| III-143 | 10.0 |
| III-144 | 6.7 |
| III-145 | 24.0 |
| III-146 | 8.7 |

TABLE 2d
MKNK1 high ATP IC₅₀s

| | MKNK1 high |
|---|---|
| Example | ATP IC₅₀ [nM] |
| IV-1 | 12 |
| IV-2 | 26 |
| IV-3 | 28 |
| IV-4 | 4 |
| IV-5 | 4 |
| IV-6 | 8 |
| IV-7 | 4 |
| IV-8 | 10 |
| IV-9 | 9 |
| IV-10 | 17 |
| IV-11 | 25 |
| IV-12 | 7 |
| IV-13 | 43 |
| IV-14 | 2 |

CDK2/CycE kinase assay

CDK2/CycE-inhibitory activity of compounds of the present invention was quantified employing the CDK2/CycE TR-FRET assay as described in the following paragraphs.

Recombinant fusion proteins of GST and human CDK2 and of GST and human CycE, expressed in insect cells (Sf9) and purified by Glutathion-Sepharose affinity chromatography, were purchased from ProQinase GmbH (Freiburg, Germany). As substrate for the kinase reaction biotinylated peptide biotin-Ttds-YISPLKSPYKISEG (C-terminus in amid form) was used which can be purchased e.g. form the company JERINI peptide technologies (Berlin, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of CDK2/CycE in aqueous assay buffer [50 mM Tris/HCl pH 8.0, 10 mM magnesium chloride, 1.0 mM dithiothreitol, 0.1 mM sodium ortho-vanadate, 0.01% (v/v) Nonidet-P40 (Sigma)] were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (1.25 μM=>final conc. in the 5 μL assay volume is 0.75 μM) in assay buffer and the resulting mixture was incubated for a reaction time of 25 min at 22° C. The concentration of CDK2/CycE was adjusted depending of the activity of the enzyme lot and was chosen appropriate to have the assay in the linear range, typical concentrations were in the range of 130 ng/ml. The reaction was stopped by the addition of 5 μL of a solution of TR-FRET detection reagents (0.2 μM streptavidine-XL665 [Cisbio Bioassays, Codolet, France] and 1 nM anti-RB(pSer807/pSer811)-antibody from BD Pharmingen [#558389] and 1.2 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (100 mM EDTA, 0.2% (w/v) bovine serum albumin in 100 mM HEPES/NaOH pH 7.0).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC50 values were calculated by a 4 parameter fit.

### PDGFRβ Kinase Assay

PDGFRβ inhibitory activity of compounds of the present invention was quantified employing the PDGFRβ HTRF assay as described in the following paragraphs.

As kinase, a GST-His fusion protein containing a C-terminal fragment of human PDGFRβ (amino acids 561-1106, expressed in insect cells [SF9] and purified by affinity chromatography, purchased from Proqinase [Freiburg i.Brsg., Germany] was used. As substrate for the kinase reaction the biotinylated poly-Glu,Tyr (4:1) copolymer (#61GTOBLA) from Cis Biointernational (Marcoule, France) was used.

For the assay 50 nL of a 100 fold concentrated solution of the test compound in DMSO was pipetted into a black low volume 384 well microtiter plate (Greiner Bio-One, Frickenhausen, Germany), 2 μL of a solution of PDGFRβ in aqueous assay buffer [50 mM HEPES/NaOH pH 7.5, 10 mM magnesium chloride, 2.5 mM dithiothreitol, 0.01% (v/v) Triton-X100 (Sigma)] were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (2.27 μg/mL=>final conc. in the 5 μL assay volume is 1.36 μg/mL [˜30 nM]) in assay buffer and the resulting mixture was incubated for a reaction time of 25 min at 22° C. The concentration of PDGFRβ in the assay was adjusted depending of the activity of the enzyme lot and was chosen appropriate to have the assay in the linear range, typical enzyme concentrations were in the range of about 125 μg/μL (final conc. in the 5 μL assay volume). The reaction was stopped by the addition of 5 μL of a solution of HTRF detection reagents (200 nM streptavidine-XLent [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture was incubated 1 h at 22° C. to allow the binding of the biotinylated phosphorylated peptide to the streptavidine-XLent and the PT66-Eu-Chelate. Subsequently the amount of phosphorylated substrate was evaluated by measurement of the resonance energy transfer from the PT66-Eu-Chelate to the streptavidine-XLent. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Normally test compound were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit.

### Fyn Kinase Assay

C-terminally His6-tagged human recombinant kinase domain of the human T-Fyn expressed in baculovirus infected insect cells (purchased from Invitrogen, P3042) was used as kinase. As substrate for the kinase reaction the biotinylated peptide biotin-KVEKIGEGTYGVV (C-terminus in amid form) was used which can be purchased e.g. form the company Biosynthan GmbH (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of T-Fyn in aqueous assay buffer [25 mM Tris/HCl pH 7.2, 25 mM magnesium chloride, 2 mM dithiothreitol, 0.1% (w/v) bovine serum albumin, 0.03% (v/v) Nonidet-P40 (Sigma)]. were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (2 μM=>final conc. in the 5 μL assay volume is 1.2 μM) in assay buffer and the resulting mixture was incubated for a reaction time of 60 min at 22° C. The concentration of Fyn was adjusted depending of the activity of the enzyme lot and was chosen appropriate to have the assay in the linear range, typical concentration was 0.13 nM. The reaction was stopped by the addition of 5 μL of a solution of HTRF detection reagents (0.2 μM streptavidine-XL [Cisbio Bioassays, Codolet, France) and 0.66 nM PT66-Eu-Chelate, an europium-chelate labelled anti-phospho-tyrosine antibody from Perkin Elmer [instead of the PT66-Eu-chelate PT66-Tb-Cryptate from Cisbio Bioassays can also be used]) in an aqueous EDTA-solution (125 mM EDTA, 0.2% (w/v) bovine serum albumin in 50 mM HEPES/NaOH pH 7.0).

The resulting mixture was incubated 1 h at 22° C. to allow the binding of the biotinylated phosphorylated peptide to the streptavidine-XL and the PT66-Eu-Chelate. Subsequently the amount of phosphorylated substrate was evaluated by measurement of the resonance energy transfer from the PT66-Eu-Chelate to the streptavidine-XL. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Normally test compounds were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit.

### Flt4 Kinase Assay

Flt4 inhibitory activity of compounds of the present invention was quantified employing the Flt4 TR-FRET assay as described in the following paragraphs.

As kinase, a GST-His fusion protein containing a C-terminal fragment of human Flt4 (amino acids 799-1298, expressed in insect cells [SF9] and purified by affinity chromatography, purchased from Proqinase [Freiburg i.Brsg., Germany] was used. As substrate for the kinase reaction the biotinylated peptide Biotin-Ahx-GGEEEEYFELVKKKK (C-terminus in amide form, purchased from Biosyntan, Berlin-Buch, Germany) was used.

For the assay 50 nL of a 100 fold concentrated solution of the test compound in DMSO was pipetted into a black low volume 384 well microtiter plate (Greiner Bio-One, Frickenhausen, Germany), 2 μL of a solution of Flt4 in aqueous assay buffer [25 mM HEPES pH 7.5, 10 mM magnesium chloride, 2 mM dithiothreitol, 0.01% (v/v) Triton-X100 (Sigma), 0.5 mM EGTA, and 5 mM R-phospho-glycerol] were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture was incubated for a reaction time of 45 min at 22° C. The concentration of Flt4 in the assay was adjusted depending of the activity of the enzyme lot and was chosen appropriate to have the assay in the linear range, typical enzyme concentrations were in the range of about 120 μg/μL (final conc. in the 5 μL assay volume). The reaction was stopped by the addition of 5 μL of a solution of HTRF detection reagents (200 nM streptavidine-XL665 [Cis Biointernational] and 1 nM PT66-Tb-Cryptate, an terbium-cryptate labelled anti-phospho-tyrosine antibody from Cisbio Bioassays (Codolet, France) in an aqueous EDTA-solution (50 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Tb-Cryptate. Subsequently the amount of phosphorylated substrate was evaluated by measurement of the resonance energy transfer from the PT66-Tb-Cryptate to the streptavidine-XL665. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Normally test compound were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit.

### TrkA Kinase Assay

TrkA inhibitory activity of compounds of the present invention was quantified employing the TrkA HTRF assay as described in the following paragraphs.

As kinase, a GST-His fusion protein containing a C-terminal fragment of human TrkA (amino acids 443-796, expressed in insect cells [SF9] and purified by affinity chromatography, purchased from Proqinase [Freiburg i.Brsg., Germany] was used. As substrate for the kinase reaction the biotinylated poly-Glu,Tyr (4:1) copolymer (#61GTOBLA) from Cis Biointernational (Marcoule, France) was used.

For the assay 50 nL of a 100 fold concentrated solution of the test compound in DMSO was pipetted into a black low volume 384 well microtiter plate (Greiner Bio-One, Frickenhausen, Germany), 2 μL of a solution of TrkA in aqueous assay buffer [8 mM MOPS/HCl pH 7.0, 10 mM magnesium chloride, 1 mM dithiothreitol, 0.01% (v/v) NP-40 (Sigma), 0.2 mM EDTA] were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (2.27 μg/mL=>final conc. in the 5 μL assay volume is 1.36 μg/mL [˜30 nM]) in assay buffer and the resulting mixture was incubated for a reaction time of 60 min at 22° C. The concentration of TrkA in the assay was adjusted depending of the activity of the enzyme lot and was chosen appropriate to have the assay in the linear range, typical enzyme concentrations were in the range of about 20 μg/μL (final conc. in the 5 μL assay volume). The reaction was stopped by the addition of 5 μL of a solution of HTRF detection reagents (30 nM streptavidine-XL665 [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture was incubated 1 h at 22° C. to allow the binding of the biotinylated phosphorylated peptide to the streptavidine-XL665 and the PT66-Eu-Chelate. Subsequently the amount of phosphorylated substrate was evaluated by measurement of the resonance energy transfer from the PT66-Eu-Chelate to the streptavidine-XL665. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Normally test compound were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit.

AlphaScreen SureFire eIF4E Ser209 Phosphorylation Assay

The AlphaScreen SureFire eIF4E Ser209 phosphorylation assay is used to measure the phosphorylation of endogenous eIF4E in cellular lysates. The AlphaScreen SureFire technology allows the detection of phosphorylated proteins in cellular lysates. In this assay, sandwich antibody complexes, which are only formed in the presence of the analyte (p-eIF4E Ser209), are captured by AlphaScreen donor and acceptor beads, bringing them into close proximity. The excitation of the donor bead provokes the release of singlet oxygen molecules that triggers a cascade of energy transfer in the Acceptor beads, resulting in the emission of light at 520-620 nm.

Surefire EIF4e Alphascreen in A549 Cells with 20% FCS Stimulation

For the assay the AlphaScreen SureFire p-eIF4E Ser209 10K Assay Kit and the AlphaScreen ProteinA Kit (for 10K assay points) both from Perkin Elmer were used.

On day one 50.000 A549 cells were plated in a 96-well plate in 100 μL per well in growth medium (DMEM/Hams' F12 with stable Glutamin, 10% FCS) and incubated at 37° C. After attachment of the cells, medium was changed to starving medium (DMEM, 0.1% FCS, without glucose, with glutamine, supplemented with 5 g/L Maltose). On day two, test compounds were serially diluted in 50 μL starving medium with a final DMSO concentration of 1% and were added to A549 cells in test plates at a final concentration range from as high 10 μM to as low 10 nM depending on the activities of the tested compounds. Treated cells were incubated at 37° C. for 2 h. 37 ul FCS was added to the wells (=final FCS concentration 20%) for 20 min. Then medium was removed and cells were lysed by adding 50 μL lysis buffer. Plates were then agitated on a plate shaker for 10 min. After 10 min lysis time, 4 μL of the lysate is transferred to a 384 well plate (Proxiplate from Perkin Elmer) and 5 μL Reaction Buffer plus Activation Buffer mix containing AlphaScreen Acceptor beads was added. Plates were sealed with TopSeal-A adhesive film, gently agitated on a plate shaker for 2 hours at room temperature. Afterwards 21 μL Dilution buffer with AlphaScreen Donor beads were added under subdued light and plates were sealed again with TopSeal-A adhesive film and covered with foil. Incubation takes place for further 2 h gently agitation at room temperature. Plates were then measured in an EnVision reader (Perkin Elmer) with the AlphaScreen program. Each data point (compound dilution) was measured as triplicate.

The IC50 values were determined by means of a 4-parameter fit.

It will be apparent to persons skilled in the art that assays for other MKNK-1 kinases may be performed in analogy using the appropriate reagents.

Thus the compounds of the present invention effectively inhibit one or more MKNK-1 kinases and are therefore suitable for the treatment or prophylaxis of diseases of uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1, more particularly in which the diseases of uncontrolled cell growth, proliferation and/or survival, inappropriate cellular immune responses, or inappropriate cellular inflammatory responses are haemotological tumours, solid tumours and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

1. A compound of general formula (I): 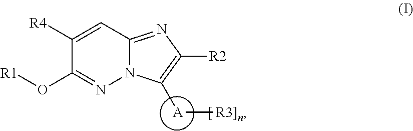
selected from: 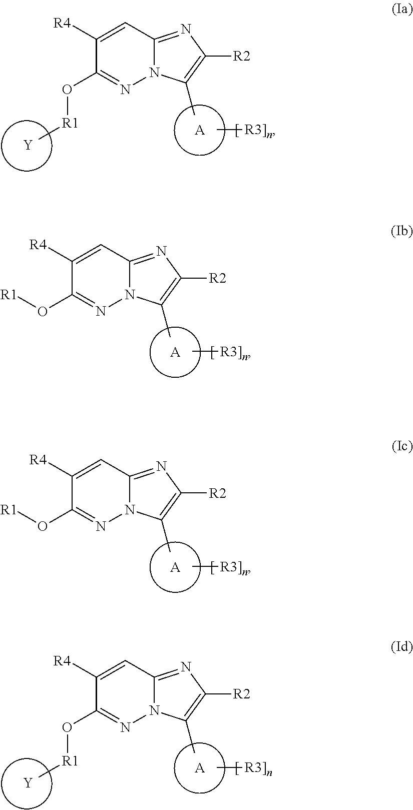
in which: 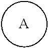
in formulae (Ia) and (Ib) represents a: 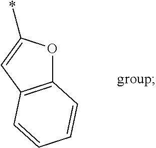 [SMILES: *c1cc2ccccc2o1] 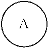
in formulae (Ic) and (Id) represents a: 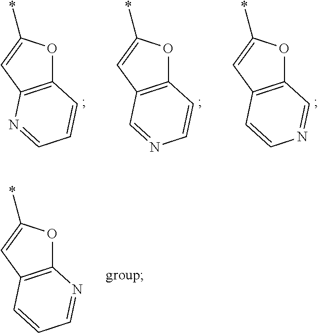 [SMILES: *c1cc2ncccc2o1 ; *c1cc2cnccc2o1 ; *c1cc2ccncc2o1 ; *c1cc2cccnc2o1]
wherein * indicates the point of attachment of said group with the rest of the molecule; and 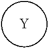
in formulae (Ia) and (Id) represents a: 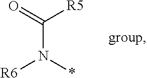
or a 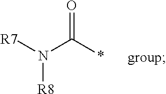
wherein * indicates the point of attachment of said groups to R1;
R1 in formula (Ia) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —N(H)S(═O)R′, —N(R1S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R1 in formulae (Ib) and (Ic) represents a substituent selected from:
a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
and
R1 in formula (Id) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R2 in formulae (Ia), (Ib), (Ic) and (Id) represents a hydrogen atom;
R3 in formulae (Ia) and (Id) represents a substituent selected from:
a halogen atom, a —CN, C₁-C₆-alkyl-, C₁-C₆-haloalkyl-, C₂-C₆-alkenyl-, C₂-C₆-alkynyl-, —C(═O)R′, —C(═O)NH₂, —C(═O)N(H)R′, —C(═O)N(R′)R″, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₃-C₆-cycloalkyl-C₁-C₆-alkoxy-, C₁-C₆-haloalkoxy-, —OC(═O)R′, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
and
R3 in formulae (Ib) and (Ic) represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 in formulae (Ia) and (Id) represents a substituent selected from:
a hydrogen atom, a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
and
R4 in formulae (Ib) and (Ic) represents a substituent selected from:
a hydrogen atom, a halogen atom, a —CN, C₁-C₆-alkyl-, C₁-C₆-haloalkyl-, C₃-C₁₀-cycloalkyl-, 3- to 10-membered heterocycloalkyl-group;
R5 in formulae (Ia) and (Id) represents a substituent selected from:
a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl- optionally substituted one or more times, independently from each other, with an R substituent;
or
together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
and
R5 in formulae (Ib) and (Ic) represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 in formulae (Ia) and (Id) represents a substituent selected from:
hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
together with a carbon atom of R1, represents a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R5 and R6 together represent a 4-, 5-, or 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R6 in formulae (Ib) and (Ic) represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 and R8 in formulae (Ia) and (Id) represents a substituent selected from:
independently from each other, a substituent selected from:
a hydrogen atom, a C₁-C₆-alkyl-, C₃-C₁₀-cycloalkyl-, C₁-C₆-haloalkyl group;
or
together, represent a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R1R″ group;
said 6- or 7-membered cyclic amine group optionally containing one further heteroatom selected from the group consisting of O, N and S;
or
R7 or R8 together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R1R″ group;
said 5-, 6- or 7-membered cyclic amide group optionally containing one further heteroatom selected from the group consisting of O, N and S;
R7 in formulae (Ib) and (Ic) represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R in formulae (Ia), (Ib), (Ic) and (Id) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ in formulae (Ia), (Ib), (Ic) and (Id) represents a substituent selected independently from each other from:
a C₁-C₆-alkyl-, C₃-C₁₀-cycloalkyl-, C₁-C₆-haloalkyl group;
R′″ and R″″ in formulae (Ib) and (Ic) represents a substituent selected independently from each other from:
a C₁-C₄-alkyl group;
R′″″ in formulae (Ib) and (Ic) represents a substituent selected from:
a C₁-C₄-alkyl group, phenyl;
n in formulae (Ia) and (Ib) represents an integer of:
0, 1, 2, 3, 4 or 5;
n in formula (Ic) represents an integer of:
1, 2, 3 or 4;
and
n in formula (Id) represents an integer of:
0, 1, 2, 3 or 4;
or a stereoisomer, a tautomer, an N-oxide, a hydrate, a solvate, or a salt thereof, or a mixture of same.

2. The compound according to claim 1, wherein: 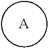
in formulae (Ia) and (Ib) represents a: 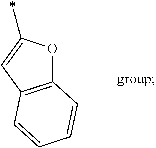 [SMILES: *c1cc2ccccc2o1] 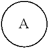
in formulae (Ic) and (Id) represents a: 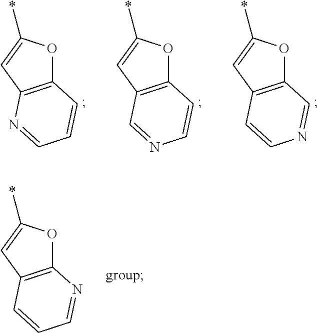 [SMILES: *c1cc2ncccc2o1 ; *c1cc2cnccc2o1 ; *c1cc2ccncc2o1 ; *c1cc2cccnc2o1]
wherein * indicates the point of attachment of said group with the rest of the molecule; and 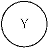
in formulae (Ia) and (Id) represents a: 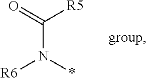
or a 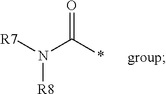
wherein * indicates the point of attachment of said groups to R1; and
R1 in formula (Ia) represents a substituent selected from:
linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R1 in formulae (Ib) and (Ic) represents a substituent selected from:
a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
and
R1 in formula (Id) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R2 in formulae (Ia), (Ib), (Ic) and (Id) represents a hydrogen atom;
R3 in formulae (Ia) and (Id) represents a substituent selected from:
a halogen atom, a —CN, C₁-C₆-alkyl-, C₁-C₆-haloalkyl-, C₂-C₆-alkenyl-, C₂-C₆-alkynyl-, —C(═O)R′, —C(═O)NH₂, —C(═O)N(H)R′, —C(═O)N(R′)R″, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₃-C₆-cycloalkyl-C₁-C₆-alkoxy-, C₁-C₆-haloalkoxy-, —OC(═O)R′, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R3 in formulae (Ib) and (Ic) represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R4 in formula (Ia) represents a substituent selected from:
a hydrogen atom, a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent
R4 in formulae (Ib) and (Ic) represents a substituent selected from:
a hydrogen atom, a halogen atom, a —CN, C₁-C₆-alkyl-, C₁-C₆-haloalkyl-, C₃-C₁₀-cycloalkyl-, 3- to 10-membered heterocycloalkyl-group;
and
R4 in formula (Id) represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 in formulae (Ia) and (Id) represents a substituent selected from:
a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl- optionally substituted one or more times, independently from each other, with an R substituent;
or
together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R5 in formulae (Ib) and (Ic) represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 in formulae (Ia) and (Id) represents a substituent selected from:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
or
together with a carbon atom of R1, represents a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S; * R5 and R6 together represent a 4-, 5-, or 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R6 in formulae (Ib) and (Ic) represent a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 and R8 in formulae (Ia) and (Id) represent a substituent selected from:
independently from each other, a substituent selected from:
a hydrogen atom, a C₁-C₆-alkyl-, C₃-C₁₀-cycloalkyl-, C₁-C₆-haloalkyl group;
or
together, represent a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R7 or R8 together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent, —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R7 in formulae (Ib) and (Ic) represent a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R in formulae (Ia) and (Id) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R in formulae (Ib) and (Ic) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ in formulae (Ia), (Ib), (Ic) and (Id) represent, independently from each other, a substituent selected from:
a C₁-C₆-alkyl-, C₃-C₁₀-cycloalkyl-, C₁-C₆-haloalkyl group;
n in formula (Ia) represents an integer of:
0, 1, 2, 3, 4 or 5;
n in formulae (Ib) and (Ic) represents an integer of:
1;
n in formula (Id) represents an integer of:
0, 1, 2, 3 or 4;
R′″ and R″″ in formulae (Ib) and (Ic) represent, independently from each other:
a C₁-C₄-alkyl group;
R′″″ in formulae (Ib) and (Ic) represents a substituent selected from:
a C₁-C₄-alkyl group, phenyl;
or a stereoisomer, a tautomer, an N-oxide, a hydrate, a solvate, or a salt thereof, or a mixture of same.

3. The compound according to claim 1 or 2, wherein: 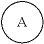
in formulae (Ia) and (Ib) represents a: 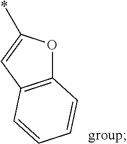 [SMILES: *c1cc2ccccc2o1] 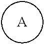
in formulae (Ic) and (Id) represents a: 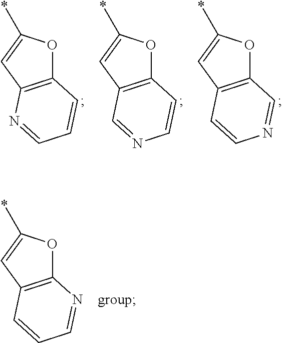
wherein * indicates the point of attachment of said group with the rest of the molecule; and 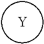
in formulae (Ia) and (Id) represents a: 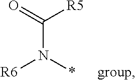
or a 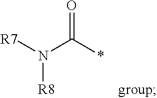
wherein * indicates the point of attachment of said groups to R1; and
R1 in formula (Ia) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
R1 in formulae (Ib) and (Ic) represents a substituent selected from:
a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —S(═O)(═N(CN))R″ group;
or a 4- to 10-membered heterocycloalkyl group optionally substituted one or more times, independently from each other with an R5 substituent;
R1 in formula (Id) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R1R″ group;
R2 in formulae (Ia), (Ib), (Ic) and (Id) represents a hydrogen atom;
R3 in formula (Ia) represents a substituent selected from:
a halogen atom, a —CN, C₁-C₆-alkyl-, C₁-C₆-haloalkyl-, —NHR′, —OH, C₁-C₆-alkoxy-, C₃-C₆-cycloalkyl-C₁-C₃-alkoxy-, C₁-C₆-haloalkoxy-
R3 in formulae (Ib) and (Ic) represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R3 in formula (Id) represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group;
R4 in formula (Ia) represents a substituent selected from:
a hydrogen atom, a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent
R4 in formulae (Ib) and (Ic) represents:
a hydrogen atom;
R4 in formula (Id) represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 in formulae (Ia) and (Id) represents a substituent selected from:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl-optionally substituted one or more times, independently from each other, with an R substituent;
or
together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R5 in formulae (Ib) and (Ic) represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 in formulae (Ia) and (Id) represents a substituent selected from:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
or
together with a carbon atom of R1, represents a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R5 and R6 together represent a 4-, 5-, or 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R6 in formulae (Ib) and (Ic) represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 and R8 represent a substituent selected from:
independently from each other, a substituent selected from:
a hydrogen atom, a C₁-C₆-alkyl-, C₃-C₁₀-cycloalkyl-, C₁-C₆-haloalkyl group;
or
together, represent a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R1R″ group;
said 6- or 7-membered cyclic amine group optionally containing one further heteroatom selected from the group consisting of O, N and S;
or
R7 or R8 together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R1R″ group;
said 5-, 6- or 7-membered cyclic amide group optionally containing one further heteroatom selected from the group consisting of O, N and S;
R7 in formulae (Ib) and (Ic) represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R in formulae (Ia) and (Id) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R in formulae (Ib) and (Ic) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ in formulae (Ia), (Ib), (Ic) and (Id), independently from each other, represents a substituent selected from:
a C₁-C₆-alkyl-, C₃-C₁₀-cycloalkyl-, C₁-C₆-haloalkyl group;
R′″ and R″″ in formulae (Ib) and (Ic), independently from each other, represents a substituent selected from:
a C₁-C₄-alkyl group;
R′″″ in formulae (Ib) and (Ic) represents a substituent selected from:
a C₁-C₄-alkyl group, phenyl;
n in formulae (Ia) and (Id) represents an integer of:
0 or 1;
n in formulae (Ib) and (Ic) represents an integer of:
1;
or a stereoisomer, a tautomer, an N-oxide, a hydrate, a solvate, or a salt thereof, or a mixture of same.

4. The compound according to any one claim 1, 2 or 3, wherein: 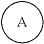
in formulae (Ia) and (Ib) represents a: 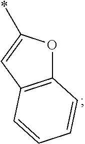 [SMILES: *c1cc2ccccc2o1] 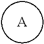
in formulae (Ic) and (Id) represents a: 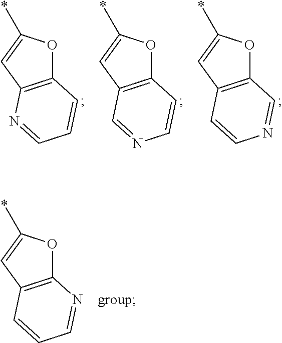
wherein * indicates the point of attachment of said group with the rest of the molecule; 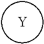
in formulae (Ia) and (Id) represents a: 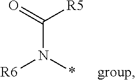
or a 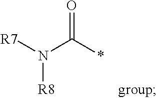
wherein * indicates the point of attachment of said group to R1;
R1 in formula (Ia) represents a substituent selected from:
linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—;
R1 in formulae (Ib) and (IC) represents a substituent selected from:
a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent;
R1 in formula (Id) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 in formulae (Ia), (Ib), (Ic) and (Id) represents a hydrogen atom;
R3 in formula (Ia) represents a substituent selected from:
a halogen atom, a —CN, C₁-C₆-alkyl-, C₁-C₆-haloalkyl-, —NHR′, —OH, C₁-C₆-alkoxy-, C₃-C₆-cycloalkyl-C₁-C₃-alkoxy-, C₁-C₆-haloalkoxy-
R3 in formulae (Ib) and (Ic) represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R3 in formula (Id) represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group;
R4 in formula (Ia) represents a substituent selected from:
a hydrogen atom, a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent
R4 in formulae (Ib) and (Ic) represents a hydrogen atom;
R4 in formula (Id) represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 in formulae (Ia) and (Id) represents a substituent selected from:
a substituent selected from a C₁-C₆-alkyl-, C₁-C₆-haloalkyl-, C₂-C₆-alkenyl-, C₂-C₆-alkynyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted one or more times, independently from each other, with an R substituent; heteroaryl-optionally substituted one or more times, independently from each other, with an R substituent;
or
together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R5 in formulae (Ib) and (Ic) represents a substituent selected from:
a halogen atom, a —CN, C₁-C₆-alkyl-, C₁-C₆-haloalkyl-, C₁-C₆-hydroxyalkyl-, C₁-C₆-alkoxy-C₁-C₆-alkyl-, C₂-C₆-alkenyl-, C₂-C₆-alkynyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″, —CH2-O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R6 in formulae (Ia) and (Id) represents a substituent selected from:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
or
together with a carbon atom of R1, represents a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R5 and R6 together represent a 4-, 5-, or 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R6 in formulae (Ib) and (Ic) represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R7 and R8 in formulae (Ia) and (Id) represent:
independently from each other, a substituent selected from:
a hydrogen atom, a C₁-C₆-alkyl-, C₃-C₁₀-cycloalkyl-, C₁-C₆-haloalkyl group;
or
together, represent a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R7 or R8 together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R7 in formulae (Ib) and (Ic) represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R in formulae (Ia) and (Id) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R in formulae (Ib) and (Ic) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ in formulae (Ia), (Ib), (Ic) and (Id) represent, independently from each other, a substituent selected from:
a C₁-C₆-alkyl-, C₃-C₁₀-cycloalkyl-, C₁-C₆-haloalkyl group;
R′″ and R″″ in formulae (Ib) and (Ic) represent, independently from each other:
a C₁-C₄-alkyl group;
R′″″ in formulae (Ib) and (Ic) represents a substituent selected from:
a C₁-C₄-alkyl group, phenyl;
n in formulae (Ia) and (Id) represents an integer of:
0 or 1;
n in formulae (Ib) and (Ic) represents an integer of:
1;
or a stereoisomer, a tautomer, an N-oxide, a hydrate, a solvate, or a salt thereof, or a mixture of same.

5. The compound according to any one of claims 1 to 4, wherein: 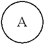
in formulae (Ia) and (Ib) represents a: 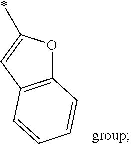 [SMILES: *c1cc2ccccc2o1] 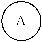
in formulae (Ic) and (Id) represents a: 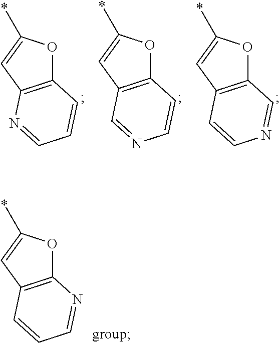
wherein * indicates the point of attachment of said group with the rest of the molecule; 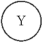
in formulae (Ia) and (Id) represents a: 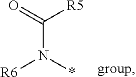
or a 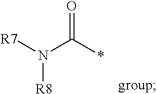
wherein * indicates the point of attachment of said group to R1; and
R1 in formula (Ia) represents a substituent selected from:
a linear C₁-C₆-alkyl-, a branched C₃-C₆-alkyl-, or a C₃-C₆-cycloalkyl group which is optionally substituted with a heteroaryl-group;
R1 in formulae (Ib) and (Ic) represents a substituent selected from:
a linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent;
R1 in formula (Id) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 in formulae (Ia), (Ib), (Ic) and (Id) represents a hydrogen atom;
R3 in formula (Ia) represents a substituent selected from:
a halogen atom or a C₁-C₆-alkoxy-group;
R3 in formulae (Ib) and (Ic) represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R3 in general formula (Id) represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group;
R4 in formulae (Ia), (Ib) and (Ic) represents a hydrogen atom;
R4 in formula (Id) represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 in formula (Ia) represents a substituent selected from:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl, heterocycloalkyl-, aryl- optionally substituted with a methyl- or chloro-group; heteroaryl- optionally substituted with a methyl-group;
or
together with a carbon atom of R1, represents a 5- or 6-membered cyclic amide group;
said 5- or 6-membered cyclic amide group optionally containing one further heteroatom selected from the group consisting of O and N;
R5 in formula (Ib) represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R/C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R5 in formula (Ic) represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R/C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R5 in formula (Id) represents a substituent selected from:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
or
together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
R6 in formula (Ia) represents a substituent selected from:
a substituent selected from hydrogen or a C₁-C₆-alkyl-group;
or
together with a carbon atom of R1, represents a 5- or 6-membered cyclic amine group;
said 6-membered cyclic amine group optionally containing one further heteroatom consisting of O;
or
R5 and R6 together represent a 5-membered cyclic amide group:
said 5-membered cyclic amide group optionally containing one further heteroatom consisting of N;
R6 in formulae (Ib) and (Ic) represents a substituent selected from:
a hydrogen atom, a C₁-C₆-alkyl-, C₃-C₁₀-cycloalkyl-, C₁-C₆-haloalkyl-, C₁-C₆-alkoxy-C₁-C₆-alkyl-, C₁-C₆-hydroxyalkyl group, a 4- to 10-membered heterocycloalkyl group optionally substituted one or more times, independently from each other with an R5 substituent;
R6 in formula (Id) represents a substituent selected from:
a substituent selected from hydrogen or a C₁-C₆-alkyl-, C₁-C₆-haloalkyl-, C₃-C₆-alkenyl-, C₃-C₆-alkynyl-, C₃-C₁₀-cycloalkyl-, C₃-C₁₀-cycloalkyl-C₁-C₆-alkyl-, aryl-optionally substituted one or more times, independently from each other, with an R substituent; heteroaryl- optionally substituted one or more times, independently from each other, with an R substituent; group;
or
together with a carbon atom of R1, represents a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R5 and R6 together represent a 4-, 5-, or 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R7 in formulae (Ib) and (Ic) represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R7 and R8 in formula (Ia) represent:
independently from each other, a substituent selected from:
a hydrogen atom or a C₁-C₆-alkyl-group;
or
R7 or R8 together with a carbon atom of R1, represents a 5-membered cyclic amide group:
R7 and R8 in general formula (Id), represent:
independently from each other, a substituent selected from:
a hydrogen atom, a C₁-C₆-alkyl-, C₃-C₁₀-cycloalkyl-, C₁-C₆-haloalkyl group;
or
together, a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R7 or R8, together with a carbon atom of R1, represent a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 5-, 6- or 7-membered cyclic amide group optionally containing one further heteroatom selected from the group consisting of O, N and S;
R in formula (Ia) represents a substituent selected from:
a halogen atom, a —CN, C₁-C₆-alkyl-, C₁-C₆-haloalkyl-group;
R in formulae (Ib) and (Ic) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R in formula (Id) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ in formulae (Ia) and (Id) represent, independently from each other, a substituent selected from:
a C₁-C₆-alkyl-group;
R′ and R″ in formulae (Ib) and (Ic) represent, independently from each other, a substituent selected from:
a C₁-C₆-alkyl-, C₃-C₁₀-cycloalkyl-, C₁-C₆-haloalkyl group;
R′″ and R″″ in formula (Ic) represents, independently from each other:
a C₁-C₄-alkyl group;
R′″″ in formula (Ic) represents a substituent selected from:
a C₁-C₄-alkyl group, phenyl;
n in formulae (Ia) and (Id) represents an integer of:
0 or 1;
n in formulae (Ib) and (Ic) represents an integer of:
1;
or a stereoisomer, a tautomer, an N-oxide, a hydrate, a solvate, or a salt thereof, or a mixture of same.

6. The compound according to claim 1, wherein: 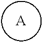
in formula (Ib) represents a: 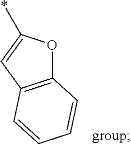 [SMILES: *c1cc2ccccc2o1] 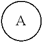
in formula (Ic) and (Id) represents a: 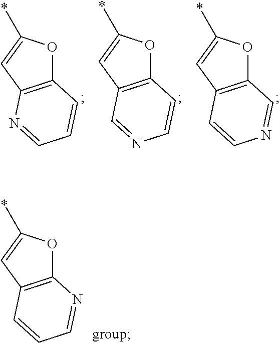
wherein * indicates the point of attachment of said group with the rest of the molecule; 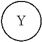
in formula (Id) represents a: 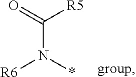
or a 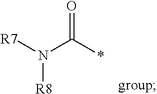
wherein * indicates the point of attachment of said group to R1; and
R1 in formulae (Ib) and (Ic) represents a substituent selected from:
linear C₁-C₆-alkyl-, a branched C₃-C₆-alkyl- or a C₃-C₁₀-cycloalkyl group; which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, 4- to 10-membered heterocycloalkyl- optionally substituted one or more times, independently from each other, with an R5 substituent; -aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)OR′, —N(R′)C(═O)OR′, N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)(═NR′)R″, —S(═O)(═N(CN))R″ group;
or a 4- to 10-membered heterocycloalkyl group optionally substituted one or more times, independently from each other with an R5 substituent;
R1 in formulae (Id) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 in formulae (Ib), (Ic) and (Id) represents a hydrogen atom;
R3 in formulae (Ib) and (Ic) represents as substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R3 in formula (Id) represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group;
R4 in formulae (Ib) and (Ic) represents a hydrogen atom;
R4 in formula (Id) represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 in formula (Ib) represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R5 in formula (Ic) represents a substituent selected from:
a C₁-C₆-alkyl-, C₁-C₆-haloalkyl-, C₁-C₆-hydroxyalkyl-, C₁-C₆-alkoxy-C₁-C₆-alkyl-, C₃-C₁₀-cycloalkyl-, 3- to 10-membered heterocycloalkyl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —OH, C₁-C₆-alkoxy-, C₁-C₆-haloalkoxy-, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group; heteroaryl- optionally substituted one or more times, independently from each other, with a halogen atom, —OH, —CN, C₁-C₆-alkyl-, C₁-C₆-haloalkyl-, C₁-C₆-alkoxy group;
R5 in formula (Id) represents a substituent selected from:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
or
together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
R6 in formulae (Ib) and (Ic) represents a substituent selected from:
a hydrogen atom, a C₁-C₆-alkyl-, C₃-C₁₀-cycloalkyl-, C₁-C₆-alkoxy-C₁-C₆-alkyl-, C₁-C₆-hydroxyalkyl group;
R6 in formula (Id) represents a substituent selected from:
a substituent selected from hydrogen or a C₁-C₆-alkyl-, C₁-C₆-haloalkyl-, C₃-C₁₀-cycloalkyl-, C₃-C₁₀-cycloalkyl-C₁-C₆-alkyl-, aryl- optionally substituted one or more times, independently from each other, with an R substituent; heteroaryl- optionally substituted one or more times, independently from each other, with an R substituent; group;
or
together with a carbon atom of R1, represents a 4-, 5-, 6- or 7-membered cyclic amine group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R5 and R6 together represent a 4-, 5-, or 6- or 7-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
R7 in formula (Ib) and (Ic) represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R7 and R8 in general formula (Id) represent:
independently from each other, a substituent selected from:
a hydrogen atom, a C₁-C₆-alkyl-, C₃-C₁₀-cycloalkyl-, C₁-C₆-haloalkyl group;
or
together, a 4-, 5-, 6- or 7-membered cyclic amine group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent, —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R1R″ group;
said 6- or 7-membered cyclic amine group optionally containing one further heteroatom selected from the group consisting of O, N and S;
or
R7 or R8, together with a carbon atom of R1, represent a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R1R″ group;
said 5-, 6- or 7-membered cyclic amide group optionally containing one further heteroatom selected from the group consisting of O, N and S;
R in formulae (Ib) and (Ic) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R in formula (Id) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ in formulae (Ib), (Ic) and (Id) represent, independently from each other, a substituent selected from:
a C₁-C₆-alkyl-, C₃-C₁₀-cycloalkyl-, C₁-C₆-haloalkyl group;
R′″ and R″″ in formula (Ic) represent, independently from each other:
a C₁-C₄-alkyl group;
R′″″ in formula (Ic) represents a substituent selected from:
a C₁-C₄-alkyl group, phenyl;
n in formulae (Ib) and (Ic) represents an integer of:
1
n in formula (Id) represents an integer of:
0 or 1;
or a stereoisomer, a tautomer, an N-oxide, a hydrate, a solvate, or a salt thereof, or a mixture of same.

7. The compound according to claim 1, wherein: 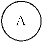
in formula (Ib) and (Ic) represents a: 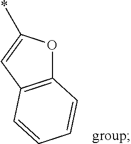 [SMILES: *c1cc2ccccc2o1] 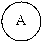
in formula (Id) represents a: 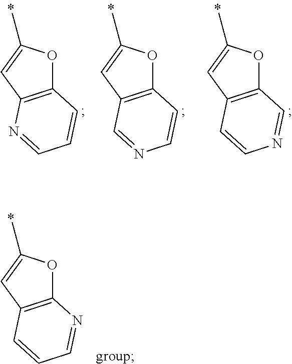
wherein * indicates the point of attachment of said group with the rest of the molecule; 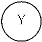
in formula (Id) represents a: 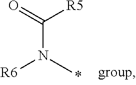
or a 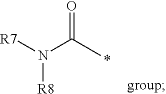
wherein * indicates the point of attachment of said group to R1;
R1 in formula (Ib) represents a substituent selected from:
linear C₁-C₆-alkyl-, or a C₃-C₁₀-cycloalkyl group; which is optionally substituted, one or more times, independently from each other, with a substituent selected from:
C₁-C₆-haloalkyl-, 4- to 10-membered heterocycloalkyl- optionally substituted one or more times, independently from each other, with an R5 substituent; —NH₂, —N(H)C(═O)OR′, —S(═O)₂R′, —S(═O)(═N(CN))R″ group;
or a 4- to 10-membered heterocycloalkyl group optionally substituted one or more times, independently from each other with an R5 substituent;
R1 in formula (Ic) represents a substituent selected from:
a linear C₁-C₆-alkyl-, or a C₃-C₁₀-cycloalkyl group; which is optionally substituted, one or more times, independently from each other, with a substituent selected from:
C₁-C₆-alkyl-, C₁-C₆-haloalkyl-, 4- to 10-membered heterocycloalkyl- optionally substituted one or more times, independently from each other, with an R5 substituent; —NH₂, —S(═O)₂R′, —S(═O)(═N(CN))R″ group;
R1 in formula (Id) represents a substituent selected from:
a linear C₁-C₆-alkyl-, a branched C₃-C₆-alkyl-, or a C₃-C₆-cycloalkyl group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 in formulae (Ib), (Ic) and (Id) represents a hydrogen atom;
R3 in formulae (Ib) and (Ic) represents a substituent selected from:
a N(R6)R7 group, or a 4- to 10-membered nitrogen atom containing heterocycloalkyl group which is optionally substituted one or more times, independently from each other with an R5 substituent, said heterocycloalkyl group being attached to the rest of the molecule via a nitrogen ring atom of the heterocycloalkyl group;
R3 in formula (Id) represents a substituent selected from:
a halogen atom, a —CN, C₁-C₆-alkyl-, C₁-C₆-haloalkyl-, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —OH, C₁-C₆-alkoxy-, C₃-C₆-cycloalkyl-C₁-C₆-alkoxy-, C₁-C₆-haloalkoxy- group;
R4 in formulae (Ib) and (Ic) represents a hydrogen atom;
R4 in formula (Id) represents a substituent selected from:
a hydrogen atom, a halogen atom, a —CN, C₁-C₆-alkyl-, C₁-C₆-haloalkyl-, C₃-C₁₀-cycloalkyl-, aryl-; heteroaryl- group;
R5 in formula (Ib) represents a substituent selected from:
a C₁-C₆-alkyl-, C₁-C₆-alkoxy-C₁-C₆-alkyl-, aryl- optionally substituted one or more times, independently from each other, with a halogen atom, —OH, —CN, C₁-C₆-alkyl-, C₁-C₆-haloalkyl-, C₁-C₆-alkoxy group;
R5 in formula (Ic) represents a substituent selected from:
a C₁-C₆-alkyl-, C₁-C₆-hydroxyalkyl-, C₁-C₆-alkoxy-C₁-C₆-alkyl-, —C(═O)R′, —C(═O)OR′, —N(R′)R″, —CH₂—O—Si(R′″)(R″″)(R′″″), aryl- optionally substituted one or more times, independently from each other, with a halogen atom, —OH, —CN, C₁-C₆-alkyl-, C₁-C₆-haloalkyl-, C₁-C₆-alkoxy group;
R5 in formula (Id) represents a substituent selected from:
a substituent selected from a C₁-C₆-alkyl-, C₁-C₆-haloalkyl-, C₃-C₁₀-cycloalkyl-, C₃-C₁₀-cycloalkyl-C₁-C₆-alkyl-, C₁-C₆-alkoxy-, C₁-C₆-alkoxy-C₁-C₆-alkyl-, aryl-C₁-C₆-alkyl-, C₁-C₆-hydroxyalkyl-, heterocycloalkyl-, aryl- optionally substituted one or more times, independently from each other, with an R substituent; heteroaryl- optionally substituted one or more times, independently from each other, with an R substituent;
or
together with a carbon atom of R1, represents a 4-, 5-, 6- or 7-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
R6 in formulae (Ib) and (Ic) represents a substituent selected from:
a C₁-C₆-alkyl group;
R6 in formula (Id) represents a substituent selected from:
a substituent selected from hydrogen or a C₁-C₆-alkyl-, C₁-C₆-haloalkyl-, C₃-C₁₀-cycloalkyl-, C₃-C₁₀-cycloalkyl-C₁-C₆-alkyl-, aryl- optionally substituted one or more times, independently from each other, with an R substituent; heteroaryl- optionally substituted one or more times, independently from each other, with an R substituent; group;
or
together with a carbon atom of R1, represents a 4-, 5-, 6- or 7-membered cyclic amine group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6- or 7-membered cyclic amine group optionally containing one further heteroatom selected from the group consisting of O, N and S;
or
R5 and R6 together represent a 4-, 5-, or 6- or 7-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
R7 in formula (Ib) represents a substituent selected from:
a C₁-C₆-alkoxy-C₁-C₆-alkyl group;
R7 in formula (Ic) represents a substituent selected from:
a C₁-C₆-alkyl group substituted with a 4- to 10-membered heterocycloalkyl group; a C₁-C₆-alkoxy-C₁-C₆-alkyl-, C₁-C₆-hydroxyalkyl-, 4- to 10-membered heterocycloalkyl group optionally substituted one or more times, independently from each other with an R5 substituent;
R7 and R8 in formula (Id) represent:
independently from each other, a substituent selected from:
a hydrogen atom, a C₁-C₆-alkyl- group;
or
together, a 4-, 5-, 6- or 7-membered cyclic amine group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 6- or 7-membered cyclic amine group optionally containing one further heteroatom selected from the group consisting of O, N and S;
or
R7 or R8, together with a carbon atom of R1, represent a 4-, 5-, 6- or 7-membered cyclic amide group, which is optionally substituted with a substituent selected from:
a halogen atom, a —CN, C₁-C₆-alkyl-, C₁-C₆-haloalkyl-, C₂-C₆-alkenyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 5-, 6- or 7-membered cyclic amide group optionally containing one further heteroatom selected from the group consisting of O, N and S;
R in formula (Id) represents a substituent selected from:
a halogen atom, a —CN, C₁-C₆-alkyl-, C₁-C₆-haloalkyl-, C₂-C₆-alkenyl-, C₂-C₆-alkynyl-, C₃-C₁₀-cycloalkyl-, 3- to 10-membered heterocycloalkyl-, aryl-, heteroaryl-, —C(═O)R′, —C(═O)NH₂, —C(═O)N(H)R′, —C(═O)N(R′)R″, —C(═O)OR′, —NH₂, —NHR′, —N(R′)R″, —N(H)C(═O)R′, —N(R′)C(═O)R′, —N(H)C(═O)NH₂, —N(H)C(═O)NHR′, —N(H)C(═O)N(R′)R″, —N(R′)C(═O)NH₂, —N(R′)C(═O)NHR′, —N(R′)C(═O)N(R′)R″, —N(H)C(═O)OR′, —N(R′)C(═O)OR′, —NO₂, —N(H)S(═O)R′, —N(R′)S(═O)R′, —N(H)S(═O)₂R′, —N(R′)S(═O)₂R′, —N═S(═O)(R′)R″, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″, —S(═O)(═NR′)R″ group;
R′ and R″ in formulae (Ib) and (Id) represent, independently from each other, a substituent selected from:
a C₁-C₆-alkyl-, C₃-C₁₀-cycloalkyl-, C₁-C₆-haloalkyl group;
R′ and R″ in formula (Ic) represent, independently from each other, a substituent selected from:
a C₁-C₆-alkyl group;
R′″ and R″″ in formula (Ic) represents, independently from each other, a C₁-C₄-alkyl group;
R′″″ in formula (Ic) represents a C₁-C₄-alkyl group;
n in formulae (Ib) and (Ic) represents an integer of:
1;
n in formula (Id) represents an integer of:
0 or 1;
or a stereoisomer, a tautomer, an N-oxide, a hydrate, a solvate, or a salt thereof, or a mixture of same.

8. The compound according to claim 1, wherein:
in formula (Id): 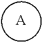
represents a: 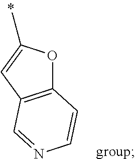
wherein * indicates the point of attachment of said group with the rest of the molecule; 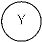
represents a: 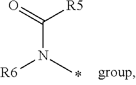
or a 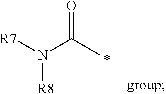
wherein * indicates the point of attachment of said group to R1;
R1 represents:
a linear C₁-C₆-alkyl- group which is optionally substituted, one or more times, independently from each other, with a substituent selected from:
a halogen atom, a —CN, C₁-C₆-alkyl-, C₁-C₆-haloalkyl-, C₃-C₁₀-cycloalkyl-, aryl- optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
R2 represents a hydrogen atom;
R3 represents a substituent selected from:
—NHR′, C₁-C₆-alkoxy-, C₃-C₆-cycloalkyl-C₁-C₆-alkoxy- group;
R4 represents a hydrogen atom;
R5 represents:
a substituent selected from a C₁-C₆-alkyl-, C₁-C₆-haloalkyl-, C₃-C₁₀-cycloalkyl
or:
together with a carbon atom of R1, represents a 6-membered cyclic amide group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S— group;
said 6-membered cyclic amide group optionally containing one further nitrogen atom;
R6 represents:
a hydrogen atom,
or:
together with a carbon atom of R1, represents a 5- or 6-membered cyclic amine group, which is optionally substituted with a substituent selected from:
a halogen atom, a —CN, C₁-C₆-alkyl-, C₁-C₆-haloalkyl-, C₃-C₁₀-cycloalkyl-, aryl-optionally substituted one or more times, independently from each other, with an R substituent; heteroaryl- optionally substituted one or more times, independently from each other, with an R substituent; —C(═O)NH₂, —C(═O)N(H)R′, —C(═O)N(R′)R″, —C(═O)OH, —C(═O)OR′, —NH₂, —NHR′, —N(R′)R″, —N(H)C(═O)R′, —N(R′)C(═O)R′, —OH, C₁-C₆-alkoxy-, C₁-C₆-haloalkoxy-, —OC(═O)R′, —OC(═O)NH₂, —OC(═O)NHR′, —OC(═O)N(R′)R″, —SH, C₁-C₆-alkyl-S—, —S(═O)R′, —S(═O)₂R′, —S(═O)₂NH₂, —S(═O)₂NHR′, —S(═O)₂N(R′)R″ group;
said 6-membered cyclic amine group optionally containing one further oxygen atom;
R7 and R8 represent:
independently from each other, a substituent selected from:
a hydrogen atom, a C₁-C₆-alkyl- group;
R represents a C₁-C₆-alkyl- group;
R′ and R″ represent, independently from each other, a substituent selected from:
a C₁-C₆-alkyl-, C₃-C₁₀-cycloalkyl-, C₁-C₆-haloalkyl group;
n represents an integer of:
0 or 1;
or a stereoisomer, a tautomer, an N-oxide, a hydrate, a solvate, or a salt thereof, or a mixture of same.

9. The compound according to any one of claims 1 to 5, which is selected from the group consisting of:
[(2S)-2-(2-{[3-(1-benzofuran-2-yl)imidazo[1,2-b]pyridazin-6-yl]oxy}ethyl)pyrrolidin-1-yl](cyclopropyl)methanone;
1-[(2S)-2-(2-{[3-(1-benzofuran-2-yl)imidazo[1,2-b]pyridazin-6-yl]oxy}ethyl)pyrrolidin-1-yl]ethanone;
1-[(2S)-2-(2-{[3-(1-benzofuran-2-yl)imidazo[1,2-b]pyridazin-6-yl]oxy}ethyl)pyrrolidin-1-yl]-2,2-dimethylpropan-1-one;
4-({[3-(1-benzofuran-2-yl)imidazo[1,2-b]pyridazin-6-yl]oxy}methyl)-1,3-oxazolidin-2-one;
N-(trans-3-{[3-(1-benzofuran-2-yl)imidazo[1,2-b]pyridazin-6-yl]oxy}cyclobutyl)cyclopropanecarboxamide;
1-[(2S)-2-(2-{[3-(1-benzofuran-2-yl)imidazo[1,2-b]pyridazin-6-yl]oxy}ethyl)pyrrolidin-1-yl]-3,3-dimethylbutan-1-one;
(5S)-5-({[3-(1-benzofuran-2-yl)imidazo[1,2-b]pyridazin-6-yl]oxy}methyl)pyrrolidin-2-one;
6-({[3-(1-benzofuran-2-yl)imidazo[1,2-b]pyridazin-6-yl]oxy}methyl)piperidin-2-one;
(5R)-5-({[3-(1-benzofuran-2-yl)imidazo[1,2-b]pyridazin-6-yl]oxy}methyl)pyrrolidin-2-one;
methyl (2S)-2-(2-{[3-(1-benzofuran-2-yl)imidazo[1,2-b]pyridazin-6-yl]oxy}ethyl)pyrrolidine-1-carboxylate;
N-(trans-3-{[3-(1-benzofuran-2-yl)imidazo[1,2-b]pyridazin-6-yl]oxy}cyclobutyl)acetamide;
1-(2-{[3-(4-methoxy-1-benzofuran-2-yl)imidazo[1,2-b]pyridazin-6-yl]oxy}ethyl)imidazolidin-2-one;
(5S)-5-({[3-(5-methoxy-1-benzofuran-2-yl)imidazo[1,2-b]pyridazin-6-yl]oxy}methyl)pyrrolidin-2-one;
1-[2-({[3-(1-benzofuran-2-yl)imidazo[1,2-b]pyridazin-6-yl]oxy}methyl)morpholin-4-yl]-3,3-dimethylbutan-1-one;
N-(2-{[3-(1-benzofuran-2-yl)imidazo[1,2-b]pyridazin-6-yl]oxy}ethyl)cyclopropanecarboxamide;
[2-({[3-(1-benzofuran-2-yl)imidazo[1,2-b]pyridazin-6-yl]oxy}methyl)morpholin-4-yl)](phenyl)methanone;
N-(2-{[3-(1-benzofuran-2-yl)imidazo[1,2-b]pyridazin-6-yl]oxy}ethyl)-3,3-dimethylbutanamide;
1-(2-{[3-(1-benzofuran-2-yl)imidazo[1,2-b]pyridazin-6-yl]oxy}ethyl)imidazolidin-2-one;
(5S)-5-({[3-(4-methoxy-1-benzofuran-2-yl)imidazo[1,2-b]pyridazin-6-yl]oxy}methyl)pyrrolidin-2-one;
2,2,2-trifluoro-1-[(2R)-2-({[3-(4-methoxy-1-benzofuran-2-yl)imidazo[1,2-b]pyridazin-6-yl]oxy}methyl)morpholin-4-yl]ethanone;
1-[(2R)-2-({[3-(4-methoxy-1-benzofuran-2-yl)imidazo[1,2-b]pyridazin-6-yl]oxy}methyl)morpholin-4-yl]-2,2-dimethylpropan-1-one;
1-(3-{[3-(1-benzofuran-2-yl)imidazo[1,2-b]pyridazin-6-yl]oxy}propyl)pyrrolidin-2-one;
N-(trans-3-{[3-(1-benzofuran-2-yl)imidazo[1,2-b]pyridazin-6-yl]oxy}cyclobutyl)-2,2,2-trifluoroacetamide;
2-{[3-(1-benzofuran-2-yl)imidazo[1,2-b]pyridazin-6-yl]oxy}acetamide;
2-{[3-(1-benzofuran-2-yl)imidazo[1,2-b]pyridazin-6-yl]oxy}propanamide;
5-(2-{[3-(1-benzofuran-2-yl)imidazo[1,2-b]pyridazin-6-yl]oxy}ethyl)pyrrolidin-2-one;
1-[2-({[3-(1-benzofuran-2-yl)imidazo[1,2-b]pyridazin-6-yl]oxy}methyl)morpholin-4-yl]ethanone;
N-(2-{[3-(1-benzofuran-2-yl)imidazo[1,2-b]pyridazin-6-yl]oxy}ethyl)acetamide;
(6S)-6-({[3-(1-benzofuran-2-yl)imidazo[1,2-b]pyridazin-6-yl]oxy}methyl)piperazin-2-one;
N-[(2R)-2-{[3-(1-benzofuran-2-yl)imidazo[1,2-b]pyridazin-6-yl]oxy}-2-(pyridin-3-yl)ethyl]-2-methoxyacetamide;
1-[(2S)-2-(2-{[3-(5-chloro-1-benzofuran-2-yl)imidazo[1,2-b]pyridazin-6-yl]oxy}ethyl)pyrrolidin-1-yl]ethanone;
(5S)-5-({[3-(1-benzofuran-2-yl)imidazo[1,2-b]pyridazin-6-yl]oxy}methyl)-1-methylpyrrolidin-2-one;
1-(2-{[3-(5-methoxy-1-benzofuran-2-yl)imidazo[1,2-b]pyridazin-6-yl]oxy}ethyl)imidazolidin-2-one;
N-[2-{[3-(1-benzofuran-2-yl)imidazo[1,2-b]pyridazin-6-yl]oxy}-1-(pyridin-3-yl)ethyl]acetamide;
N-[(2R)-2-{[3-(1-benzofuran-2-yl)imidazo[1,2-b]pyridazin-6-yl]oxy}-2-(pyridin-3-yl)ethyl]acetamide;
2-{[3-(1-benzofuran-2-yl)imidazo[1,2-b]pyridazin-6-yl]oxy}-N,N-dimethylacetamide;
2-{[3-(1-benzofuran-2-yl)imidazo[1,2-b]pyridazin-6-yl]oxy}-N-tert-butylacetamide;
3-{[3-(1-benzofuran-2-yl)imidazo[1,2-b]pyridazin-6-yl]oxy}pyrrolidin-2-one;
2-{[3-(1-benzofuran-2-yl)imidazo[1,2-b]pyridazin-6-yl]oxy}-2-(pyridin-3-yl)acetamide;
1-[(2S)-2-(2-{[3-(5-chloro-1-benzofuran-2-yl)imidazo[1,2-b]pyridazin-6-yl]oxy}ethyl)pyrrolidin-1-yl]-2,2-dimethylpropan-1-one;
cyclopropyl[(2R)-2-({[3-(4-methoxy-1-benzofuran-2-yl)imidazo[1,2-b]pyridazin-6-yl]oxy}methyl)morpholin-4-yl]methanone;
(6R)-6-({[3-(1-benzofuran-2-yl)imidazo[1,2-b]pyridazin-6-yl]oxy}methyl)piperazin-2-one;
[2-(2-{[3-(1-benzofuran-2-yl)imidazo[1,2-b]pyridazin-6-yl]oxy}ethyl)pyrrolidin-1-yl](thiophen-2-yl)methanone;
1-[2-(2-{[3-(1-benzofuran-2-yl)imidazo[1,2-b]pyridazin-6-yl]oxy}ethyl)pyrrolidin-1-yl]propan-1-one;
[2-(2-{[3-(1-benzofuran-2-yl)imidazo[1,2-b]pyridazin-6-yl]oxy}ethyl)pyrrolidin-1-yl](1,2-oxazol-4-yl)methanone;
1-[2-(2-{[3-(1-benzofuran-2-yl)imidazo[1,2-b]pyridazin-6-yl]oxy}ethyl)pyrrolidin-1-yl]-2,2-dimethylpropan-1-one;
[2-(2-{[3-(1-benzofuran-2-yl)imidazo[1,2-b]pyridazin-6-yl]oxy}ethyl)pyrrolidin-1-yl](1,2-oxazol-5-yl)methanone;
[2-(2-{[3-(1-benzofuran-2-yl)imidazo[1,2-b]pyridazin-6-yl]oxy}ethyl)pyrrolidin-1-yl](1-methyl-1H-pyrazol-4-yl)methanone;
[2-(2-{[3-(1-benzofuran-2-yl)imidazo[1,2-b]pyridazin-6-yl]oxy}ethyl)pyrrolidin-1-yl](furan-2-yl)methanone;
[2-(2-{[3-(1-benzofuran-2-yl)imidazo[1,2-b]pyridazin-6-yl]oxy}ethyl)pyrrolidin-1-yl](1-methyl-1H-pyrazol-3-yl)methanone;
1-[2-(2-{[3-(1-benzofuran-2-yl)imidazo[1,2-b]pyridazin-6-yl]oxy}ethyl)pyrrolidin-1-yl]-2-cyclopropylethanone;
[2-(2-{[3-(1-benzofuran-2-yl)imidazo[1,2-b]pyridazin-6-yl]oxy}ethyl)pyrrolidin-1-yl](3-methyl-1,2-oxazol-4-yl)methanone;
[2-(2-{[3-(1-benzofuran-2-yl)imidazo[1,2-b]pyridazin-6-yl]oxy}ethyl)pyrrolidin-1-yl](tetrahydrofuran-2-yl)methanone;
[2-(2-{[3-(1-benzofuran-2-yl)imidazo[1,2-b]pyridazin-6-yl]oxy}ethyl)pyrrolidin-1-yl](cyclobutyl)methanone;
[2-(2-{[3-(1-benzofuran-2-yl)imidazo[1,2-b]pyridazin-6-yl]oxy}ethyl)pyrrolidin-1-yl](1,2-oxazol-3-yl)methanone;
[2-(2-{[3-(1-benzofuran-2-yl)imidazo[1,2-b]pyridazin-6-yl]oxy}ethyl)pyrrolidin-1-yl](cyclopentyl)methanone;
[2-(2-{[3-(1-benzofuran-2-yl)imidazo[1,2-b]pyridazin-6-yl]oxy}ethyl)pyrrolidin-1-yl](thiophen-3-yl)methanone;
1-[2-(2-{[3-(1-benzofuran-2-yl)imidazo[1,2-b]pyridazin-6-yl]oxy}ethyl)pyrrolidin-1-yl]-2-methoxyethanone;
1-[2-(2-{[3-(1-benzofuran-2-yl)imidazo[1,2-b]pyridazin-6-yl]oxy}ethyl)pyrrolidin-1-yl]-2-hydroxyethanone;
1-[2-(2-{[3-(1-benzofuran-2-yl)imidazo[1,2-b]pyridazin-6-yl]oxy}ethyl)pyrrolidin-1-yl]-3-methylbutan-1-one;
[2-(2-{[3-(1-benzofuran-2-yl)imidazo[1,2-b]pyridazin-6-yl]oxy}ethyl)pyrrolidin-1-yl](1H-pyrrol-2-yl)methanone;
[2-(2-{[3-(1-benzofuran-2-yl)imidazo[1,2-b]pyridazin-6-yl]oxy}ethyl)pyrrolidin-1-yl](pyridin-2-yl)methanone;
[2-(2-{[3-(1-benzofuran-2-yl)imidazo[1,2-b]pyridazin-6-yl]oxy}ethyl)pyrrolidin-1-yl](4-chlorophenyl)methanone;
N-(2-{[3-(1-benzofuran-2-yl)imidazo[1,2-b]pyridazin-6-yl]oxy}ethyl)thiophene-2-carboxamide
N-(2-{[3-(1-benzofuran-2-yl)imidazo[1,2-b]pyridazin-6-yl]oxy}ethyl)-2-chlorobenzamide;
N-(2-{[3-(1-benzofuran-2-yl)imidazo[1,2-b]pyridazin-6-yl]oxy}ethyl)tetrahydro-2H-pyran-4-carboxamide;
N-(2-{[3-(1-benzofuran-2-yl)imidazo[1,2-b]pyridazin-6-yl]oxy}ethyl)-2-hydroxyacetamide;
N-(2-{[3-(1-benzofuran-2-yl)imidazo[1,2-b]pyridazin-6-yl]oxy}ethyl)cyclobutanecarboxamide
N-(2-{[3-(1-benzofuran-2-yl)imidazo[1,2-b]pyridazin-6-yl]oxy}ethyl)-3-methylbenzamide;
N-(2-{[3-(1-benzofuran-2-yl)imidazo[1,2-b]pyridazin-6-yl]oxy}ethyl)pyridine-2-carboxamide;
N-(2-{[3-(1-benzofuran-2-yl)imidazo[1,2-b]pyridazin-6-yl]oxy}ethyl)-2-methylbenzamide;
N-(2-{[3-(1-benzofuran-2-yl)imidazo[1,2-b]pyridazin-6-yl]oxy}ethyl)-3-methylbutanamide;
N-(2-{[3-(1-benzofuran-2-yl)imidazo[1,2-b]pyridazin-6-yl]oxy}ethyl)-2-methylbutanamide;
N-(2-{[3-(1-benzofuran-2-yl)imidazo[1,2-b]pyridazin-6-yl]oxy}ethyl)pentanamide;
N-(2-{[3-(1-benzofuran-2-yl)imidazo[1,2-b]pyridazin-6-yl]oxy}ethyl)-2-phenylacetamide;
N-(2-{[3-(1-benzofuran-2-yl)imidazo[1,2-b]pyridazin-6-yl]oxy}ethyl)-2-cyclopropylacetamide;
N-(2-{[3-(1-benzofuran-2-yl)imidazo[1,2-b]pyridazin-6-yl]oxy}ethyl)furan-2-carboxamide;
N-(2-{[3-(1-benzofuran-2-yl)imidazo[1,2-b]pyridazin-6-yl]oxy}ethyl)tetrahydrofuran-2-carboxamide;
N-(2-{[3-(1-benzofuran-2-yl)imidazo[1,2-b]pyridazin-6-yl]oxy}ethyl)-1-methyl-1H-pyrazole-3-carboxamide; and
N-[(2R)-2-{[3-(1-benzofuran-2-yl)imidazo[1,2-b]pyridazin-6-yl]oxy}-propyl]acetamide;
3-[4-(Morpholin-4-yl)-1-benzofuran-2-yl]-6-[(2R)-morpholin-2-ylmethoxy]imidazo[1,2-b]pyridazine;
(2S)-1-({3-[4-(Morpholin-4-yl)-1-benzofuran-2-yl]imidazo[1,2-b]pyridazin-6-yl}oxy)propan-2-amine;
tert-Butyl [trans-3-({3-[4-(morpholin-4-yl)-1-benzofuran-2-yl]imidazo[1,2-b]-pyridazin-6-yl}oxy)cyclobutyl]carbamate;
trans-3-({3-[4-(Morpholin-4-yl)-1-benzofuran-2-yl]imidazo[1,2-b]pyridazin-6-yl}oxy)-cyclobutanamine;
(5R)-5-[({3-[4-(Morpholin-4-yl)-1-benzofuran-2-yl]imidazo[1,2-b]pyridazin-6-yl}oxy)-methyl]pyrrolidin-2-one;
3-{4-[(2R)-2-(Methoxymethyl)pyrrolidin-1-yl]-1-benzofuran-2-yl}-6-[3-(methyl-sulfonyl)propoxy]imidazo[1,2-b]pyridazine;
3-{4-[(2R)-2-(Methoxymethyl)pyrrolidin-1-yl]-1-benzofuran-2-yl}-6-[(2R)-morpholin-2-ylmethoxy]imidazo[1,2-b]pyridazine;
(2S)-1-[(3-{4-[(2R)-2-(Methoxymethyl)pyrrolidin-1-yl]-1-benzofuran-2-yl}-imidazo[1,2-b]pyridazin-6-yl)oxy]propan-2-amine;
(5R)-5-{[(3-{4-[(2R)-2-(Methoxymethyl)pyrrolidin-1-yl]-1-benzofuran-2-yl}imidazo[1,2-b]pyridazin-6-yl)oxy]methyl}pyrrolidin-2-one;
6-Methoxy-3-{4-[(2R)-2-(methoxymethyl)pyrrolidin-1-yl]-1-benzofuran-2-yl}-imidazo[1,2-b]pyridazine;
trans-3-[(3-{4-[(2R)-2-(Methoxymethyl)pyrrolidin-1-yl]-1-benzofuran-2-yl}imidazo[1,2-b]pyridazin-6-yl)oxy]cyclobutanamine;
6-[3-(Methylsulfonyl)propoxy]-3-[4-(4-phenylpiperazin-1-yl)-1-benzofuran-2-yl]imidazo[1,2-b]pyridazine;
3-[4-(Morpholin-4-yl)-1-benzofuran-2-yl]-6-[(3R)-pyrrolidin-3-yloxy]-imidazo[1,2-b]pyridazine;
(5R)-5-[({3-[4-(4-Phenylpiperazin-1-yl)-1-benzofuran-2-yl]imidazo[1,2-b]-pyridazin-6-yl}oxy)methyl]pyrrolidin-2-one;
(2S)-1-({3-[4-(4-Phenylpiperazin-1-yl)-1-benzofuran-2-yl]imidazo[1,2-b]-pyridazin-6-yl}oxy)propan-2-amine;
trans-3-({3-[4-(4-Phenylpiperazin-1-yl)-1-benzofuran-2-yl]imidazo[1,2-b]-pyridazin-6-yl}oxy)cyclobutanamine;
(5R)-5-{[(3-{4-[Ethyl(2-methoxyethyl)amino]-1-benzofuran-2-yl}imidazo[1,2-b]-pyridazin-6-yl)oxy]methyl}pyrrolidin-2-one;
(5R)-5-[({3-[4-(4-Methylpiperazin-1-yl)-1-benzofuran-2-yl]imidazo[1,2-b]-pyridazin-6-yl}oxy)methyl]pyrrolidin-2-one;
(5R)-5-[({3-[4-(Piperazin-1-yl)-1-benzofuran-2-yl]imidazo[1,2-b]pyridazin-6-yl}oxy)-methyl]pyrrolidin-2-one;
6-[3-(Methylsulfonyl)propoxy]-3-[4-(piperazin-1-yl)-1-benzofuran-2-yl]imidazo-[1,2-b]pyridazine;
6-Methoxy-3-[4-(piperazin-1-yl)-1-benzofuran-2-yl]imidazo[1,2-b]pyridazine;
6-Methoxy-3-[4-(4-phenylpiperazin-1-yl)-1-benzofuran-2-yl]imidazo[1,2-b]-pyridazine;
N-Ethyl-N-(2-methoxyethyl)-2-{6-[3-(methylsulfonyl)propoxy]imidazo[1,2-b]-pyridazin-3-yl}-1-benzofuran-4-amine;
2-(6-{[(2S)-2-Aminopropyl]oxy}imidazo[1,2-b]pyridazin-3-yl)-N-ethyl-N-(2-methoxyethyl)-1-benzofuran-4-amine;
[{3-[(3-{4-[2-(Methoxymethyl)pyrrolidin-1-yl]-1-benzofuran-2-yl}imidazo[1,2-b] pyridazin-6-yl)oxy]propyl}(methyl)oxido-λ⁶-sulfanylidene]cyanamide;
(2R)-1-[(3-{4-[(2R)-2-(Methoxymethyl)pyrrolidin-1-yl]-1-benzofuran-2-yl}imidazo[1,2-b]pyridazin-6-yl)oxy]propan-2-amine;
(5R)-5-{[(3-{4-[(2S)-2-Methylmorpholin-4-yl]-1-benzofuran-2-yl}imidazo[1,2-b]pyridazin-6-yl)oxy]methyl}pyrrolidin-2-one;
6-Methoxy-3-{4-[(2S)-2-methylpiperazin-1-yl]-1-benzofuran-2-yl}imidazo[1,2-b]pyridazine;
3-{4-[(2S)-2-Methylpiperazin-1-yl]-1-benzofuran-2-yl}-6-(2,2,2-trifluoroethoxy)imidazo[1,2-b]pyridazine;
(5R)-5-{[(3-{4-[(2S)-2-Methylpiperazin-1-yl]-1-benzofuran-2-yl}imidazo[1,2-b]pyridazin-6-yl)oxy]methyl}pyrrolidin-2-one;
(2S)-1-[(3-{4-[(3S)-3-Methylmorpholin-4-yl]-1-benzofuran-2-yl}imidazo[1,2-b]pyridazin-6-yl)oxy]propan-2-amine;
(5R)-5-{[(3-{4-[(3S)-3-Methylmorpholin-4-yl]-1-benzofuran-2-yl}imidazo[1,2-b]pyridazin-6-yl)oxy]methyl}pyrrolidin-2-one;
(5S)-5-{[(3-{4-[(3S)-3-Methylmorpholin-4-yl]-1-benzofuran-2-yl}imidazo[1,2-b]pyridazin-6-yl)oxy]methyl}pyrrolidin-2-one;
trans-3-[(3-{4-[(3S)-3-Methylmorpholin-4-yl]-1-benzofuran-2-yl}imidazo[1,2-b]pyridazin-6-yl)oxy]cyclobutanamine;
(2R)-1-({3-[4-(2,2-Dimethylmorpholin-4-yl)-1-benzofuran-2-yl]imidazo[1,2-b]pyridazin-6-yl}oxy)propan-2-amine;
(5S)-5-[({3-[4-(3,3-Dimethylpiperazin-1-yl)-1-benzofuran-2-yl]imidazo[1,2-b]pyridazin-6-yl}oxy)methyl]pyrrolidin-2-one;
(2R)-1-[(3-{4-[(3R)-3-Methylmorpholin-4-yl]-1-benzofuran-2-yl}imidazo[1,2-b]pyridazin-6-yl)oxy]propan-2-amine;
(5S)-5-{[(3-{4-[(2S)-2-Methylpiperazin-1-yl]-1-benzofuran-2-yl}imidazo[1,2-b]pyridazin-6-yl)oxy]methyl}pyrrolidin-2-one;
6-Methoxy-3-{4-[(3R)-3-methylpiperazin-1-yl]-1-benzofuran-2-yl}imidazo[1,2-b]pyridazine;
6-Methoxy-3-{4-[(3S)-3-methylpiperazin-1-yl]-1-benzofuran-2-yl}imidazo[1,2-b]pyridazine;
3-{4-[(3S)-3-Methylpiperazin-1-yl]-1-benzofuran-2-yl}-6-(2,2,2-trifluoroethoxy)imidazo[1,2-b]pyridazine;
(5S)-5-{[(3-{4-[(3S)-3-Methylpiperazin-1-yl]-1-benzofuran-2-yl}imidazo[1,2-b]pyridazin-6-yl)oxy]methyl}pyrrolidin-2-one;
trans-3-[(3-{4-[(2S)-2-Methylmorpholin-4-yl]-1-benzofuran-2-yl}imidazo[1,2-b]pyridazin-6-yl)oxy]cyclobutanamine;
6-Methoxy-3-{4-[(2R)-2-methylpiperazin-1-yl]-1-benzofuran-2-yl}imidazo[1,2-b]pyridazine;
3-{4-[(2R)-2-Methylpiperazin-1-yl]-1-benzofuran-2-yl}-6-[3-(methylsulfonyl)-propoxy]imidazo[1,2-b]pyridazine;
(5R)-5-[({3-[4-(3,3-Dimethylpiperazin-1-yl)-1-benzofuran-2-yl]imidazo[1,2-b]pyridazin-6-yl}oxy)methyl]pyrrolidin-2-one;
(6S)-6-{[(3-{4-[(2R)-2-(Methoxymethyl)pyrrolidin-1-yl]-1-benzofuran-2-yl}imidazo[1,2-b]pyridazin-6-yl)oxy]methyl}piperazin-2-one;
(6R)-6-{[(3-{4-[(2R)-2-(Methoxymethyl)pyrrolidin-1-yl]-1-benzofuran-2-yl}imidazo[1,2-b]pyridazin-6-yl)oxy]methyl}piperazin-2-one;
(5R)-5-{[(3-{4-[(3R)-3-Methylmorpholin-4-yl]-1-benzofuran-2-yl}imidazo[1,2-b]pyridazin-6-yl)oxy]methyl}pyrrolidin-2-one;
trans-3-[(3-{4-[(3R)-3-Methylmorpholin-4-yl]-1-benzofuran-2-yl}imidazo[1,2-b]pyridazin-6-yl)oxy]cyclobutanamine;
(5S)-5-{[(3-{4-[(3R)-3-Methylmorpholin-4-yl]-1-benzofuran-2-yl}imidazo[1,2-b]pyridazin-6-yl)oxy]methyl}pyrrolidin-2-one;
3-{4-[(3S)-3-Methylpiperazin-1-yl]-1-benzofuran-2-yl}-6-[3-(methylsulfonyl)-propoxy]imidazo[1,2-b]pyridazine;
(5R)-5-{[(3-{4-[(3S)-3-Methylpiperazin-1-yl]-1-benzofuran-2-yl}imidazo[1,2-b]pyridazin-6-yl)oxy]methyl}pyrrolidin-2-one;
3-[4-(3,3-Dimethylpiperazin-1-yl)-1-benzofuran-2-yl]-6-(2,2,2-trifluoroethoxy)imidazo[1,2-b]pyridazine;
3-[4-(3,3-Dimethylpiperazin-1-yl)-1-benzofuran-2-yl]-6-[3-(methylsulfonyl)-propoxy]imidazo[1,2-b]pyridazine;
3-[4-(Piperazin-1-yl)-1-benzofuran-2-yl]-6-(2,2,2-trifluoroethoxy)imidazo[1,2-b]pyridazine;
(2S)-1-[(3-{4-[(3R)-3-Methylmorpholin-4-yl]-1-benzofuran-2-yl}imidazo[1,2-b]pyridazin-6-yl)oxy]propan-2-amine;
3-{4-[(2S)-2-Methylpiperazin-1-yl]-1-benzofuran-2-yl}-6-[3-(methylsulfonyl)-propoxy]imidazo[1,2-b]pyridazine;
3-{4-[(3R)-3-Methylpiperazin-1-yl]-1-benzofuran-2-yl}-6-(2,2,2-trifluoroethoxy)imidazo[1,2-b]pyridazine;
3-[4-(3,3-Dimethylpiperazin-1-yl)-1-benzofuran-2-yl]-6-methoxyimidazo[1,2-b]pyridazine;
(2S)-1-({3-[4-(2,2-Dimethylmorpholin-4-yl)-1-benzofuran-2-yl]imidazo[1,2-b]pyridazin-6-yl}oxy)propan-2-amine;
(5S)-5-{[(3-{4-[(2R)-2-Methylpiperazin-1-yl]-1-benzofuran-2-yl}imidazo[1,2-b]pyridazin-6-yl)oxy]methyl}pyrrolidin-2-one;
(5S)-5-[({3-[4-(2,2-Dimethylmorpholin-4-yl)-1-benzofuran-2-yl]imidazo[1,2-b]pyridazin-6-yl}oxy)methyl]pyrrolidin-2-one;
3-{4-[(2R)-2-Methylpiperazin-1-yl]-1-benzofuran-2-yl}-6-(2,2,2-trifluoroethoxy)imidazo[1,2-b]pyridazine;
(5R)-5-{[(3-{4-[(2R)-2-Methylpiperazin-1-yl]-1-benzofuran-2-yl}imidazo[1,2-b]pyridazin-6-yl)oxy]methyl}pyrrolidin-2-one;
trans-3-({3-[4-(2,2-Dimethylmorpholin-4-yl)-1-benzofuran-2-yl]imidazo[1,2-b]pyridazin-6-yl}oxy)cyclobutanamine;
(2S)-1-[(3-{4-[(2S)-2-Methylmorpholin-4-yl]-1-benzofuran-2-yl}imidazo[1,2-b]pyridazin-6-yl)oxy]propan-2-amine;
(5S)-5-{[(3-{4-[(2S)-2-Methylmorpholin-4-yl]-1-benzofuran-2-yl}imidazo[1,2-b]pyridazin-6-yl)oxy]methyl}pyrrolidin-2-one;
(2R)-1-[(3-{4-[(2R)-2-Methylmorpholin-4-yl]-1-benzofuran-2-yl}imidazo[1,2-b]pyridazin-6-yl)oxy]propan-2-amine;
(2S)-1-[(3-{4-[(2R)-2-Methylmorpholin-4-yl]-1-benzofuran-2-yl}imidazo[1,2-b]pyridazin-6-yl)oxy]propan-2-amine;
(5R)-5-{[(3-{4-[(2R)-2-Methylmorpholin-4-yl]-1-benzofuran-2-yl}imidazo[1,2-b]pyridazin-6-yl)oxy]methyl}pyrrolidin-2-one;
(5S)-5-{[(3-{4-[(2R)-2-Methylmorpholin-4-yl]-1-benzofuran-2-yl}imidazo[1,2-b]pyridazin-6-yl)oxy]methyl}pyrrolidin-2-one;
trans-3-[(3-{4-[(2R)-2-Methylmorpholin-4-yl]-1-benzofuran-2-yl}imidazo[1,2-b]pyridazin-6-yl)oxy]cyclobutanamine;
(5R)-5-[({3-[4-(2,2-Dimethylmorpholin-4-yl)-1-benzofuran-2-yl]imidazo[1,2-b]pyridazin-6-yl}oxy)methyl]pyrrolidin-2-one;
(2R)-1-[(3-{4-[(3S)-3-Methylmorpholin-4-yl]-1-benzofuran-2-yl}imidazo[1,2-b]pyridazin-6-yl)oxy]propan-2-amine;
(2R)-1-[(3-{4-[(2S)-2-Methylmorpholin-4-yl]-1-benzofuran-2-yl}imidazo[1,2-b]pyridazin-6-yl)oxy]propan-2-amine;
(2R)-1-({3-[4-(Morpholin-4-yl)furo[3,2-c]pyridin-2-yl]imidazo[1,2-b]pyridazin-6-yl}-oxy)propan-2-amine;
{1-[2-(6-{[(2R)-2-Aminopropyl]oxy}imidazo[1,2-b]pyridazin-3-yl)furo[3,2-c]pyridin-4-yl]piperidin-4-yl}methanol;
(2R)-1-[(3-{4-[(3R)-3-Methylmorpholin-4-yl]furo[3,2-c]pyridin-2-yl}imidazo[1,2-b]pyridazin-6-yl)oxy]propan-2-amine;
2-(6-{[(2R)-2-Aminopropyl]oxy}imidazo[1,2-b]pyridazin-3-yl)-N-ethyl-N-(2-methoxy-ethyl)furo[3,2-c]pyridin-4-amine;
(2R)-1-[(3-{4-[(3S)-3-Methylmorpholin-4-yl]furo[3,2-c]pyridin-2-yl}imidazo[1,2-b]pyridazin-6-yl)oxy]propan-2-amine;
(2R)-1-[(3-{4-[(2S)-2-Methylmorpholin-4-yl]furo[3,2-c]pyridin-2-yl}imidazo[1,2-b]pyridazin-6-yl)oxy]propan-2-amine;
(2S)-1-({3-[4-(Morpholin-4-yl)furo[3,2-c]pyridin-2-yl]imidazo[1,2-b]pyridazin-6-yl}-oxy)propan-2-amine;
(2S)-1-({3-[4-(4-Methylpiperazin-1-yl)furo[3,2-c]pyridin-2-yl]imidazo[1,2-b]pyridazin-6-yl}oxy)propan-2-amine;
(2S)-1-({3-[4-(Piperidin-1-yl)furo[3,2-c]pyridin-2-yl]imidazo[1,2-b]pyridazin-6-yl}oxy)propan-2-amine;
(2S)-1-({3-[4-(Pyrrolidin-1-yl)furo[3,2-c]pyridin-2-yl]imidazo[1,2-b]pyridazin-6-yl}oxy)propan-2-amine;
(3R)-1-[2-(6-{[(2S)-2-Aminopropyl]oxy}imidazo[1,2-b]pyridazin-3-yl)furo[3,2-c]pyridin-4-yl]-N,N-dimethylpyrrolidin-3-amine
2-(6-{[(2S)-2-Aminopropyl]oxy}imidazo[1,2-b]pyridazin-3-yl)-N-methyl-N-[3-(pyrrolidin-1-yl)propyl]furo[3,2-c]pyridin-4-amine;
2-(6-{[(2S)-2-Aminopropyl]oxy}imidazo[1,2-b]pyridazin-3-yl)-N-methyl-N-(1-methylpiperidin-4-yl)furo[3,2-c]pyridin-4-amine;
{(2R)-1-[2-(6-{[(2S)-2-Aminopropyl]oxy}imidazo[1,2-b]pyridazin-3-yl)furo[3,2-c]pyridin-4-yl]pyrrolidin-2-yl}methanol;
tert-Butyl 4-[2-(6-{[(2S)-2-aminopropyl]oxy}imidazo[1,2-b]pyridazin-3-yl)furo-[3,2-c]pyridin-4-yl]piperazine-1-carboxylate;
2-(6-{[(2S)-2-Aminopropyl]oxy}imidazo[1,2-b]pyridazin-3-yl)-N-(2-methoxyethyl)-N-methylfuro[3,2-c]pyridin-4-amine;
(2S)-1-({3-[4-(Piperazin-1-yl)furo[3,2-c]pyridin-2-yl]imidazo[1,2-b]pyridazin-6-yl}oxy)propan-2-amine;
2-(6-{[(2S)-2-Aminopropyl]oxy}imidazo[1,2-b]pyridazin-3-yl)-N-ethyl-N-(2-methoxy-ethyl)furo[3,2-c]pyridin-4-amine;
(2S)-1-[(3-{4-[(2R,6S)-2,6-Dimethylmorpholin-4-yl]furo[3,2-c]pyridin-2-yl}imidazo[1,2-b]pyridazin-6-yl)oxy]propan-2-amine;
3-{[2-(6-{[(2S)-2-Aminopropyl]oxy}imidazo[1,2-b]pyridazin-3-yl)furo[3,2-c]pyridin-4-yl](methyl)amino}propan-1-ol;
1-[2-(6-{[(2S)-2-Aminopropyl]oxy}imidazo[1,2-b]pyridazin-3-yl)furo[3,2-c]pyridin-4-yl]-N,N-dimethylpipendin-4-amine;
{1-[2-(6-{[(2S)-2-Aminopropyl]oxy}imidazo[1,2-b]pyridazin-3-yl)furo[3,2-c]pyridin-4-yl]piperidin-4-yl}methanol;
(2S)-1-({3-[4-(4-Phenylpiperazin-1-yl)furo[3,2-c]pyridin-2-yl]imidazo[1,2-b]pyridazin-6-yl}oxy)propan-2-amine;
(2S)-1-[(3-{4-[(2R)-2-(Methoxymethyl)pyrrolidin-1-yl]furo[3,2-c]pyridin-2-yl}imidazo[1,2-b]pyridazin-6-yl)oxy]propan-2-amine;
(2S)-1-[(3-{4-[(3R)-3-Methylmorpholin-4-yl]furo[3,2-c]pyridin-2-yl}imidazo[1,2-b]pyridazin-6-yl)oxy]propan-2-amine;
2-(6-{[(2S)-2-Aminopropyl]oxy}imidazo[1,2-b]pyridazin-3-yl)-N-(2-tert-butoxyethyl)-N-ethylfuro[3,2-c]pyridin-4-amine;
(2S)-1-[(3-{4-[(2S)-2-(Methoxymethyl)pyrrolidin-1-yl]furo[3,2-c]pyridin-2-yl}imidazo[1,2-b]pyridazin-6-yl)oxy]propan-2-amine;
(2S)-1-[(3-{4-[(3S)-3-Methylmorpholin-4-yl]furo[3,2-c]pyridin-2-yl}imidazo[1,2-b]pyridazin-6-yl)oxy]propan-2-amine;
(2S)-1-[(3-{4-[(2S)-2-Methylmorpholin-4-yl]furo[3,2-c]pyridin-2-yl}imidazo[1,2-b]pyridazin-6-yl)oxy]propan-2-amine;
(2R)-2-({3-[4-(Morpholin-4-yl)furo[3,2-c]pyridin-2-yl]imidazo[1,2-b]pyridazin-6-yl}-oxy)propan-1-amine;
(2R)-2-[(3-{4-[(2R)-2-(Methoxymethyl)pyrrolidin-1-yl]furo[3,2-c]pyridin-2-yl}imidazo[1,2-b]pyridazin-6-yl)oxy]propan-1-amine;
2-(6-{[(2R)-1-Aminopropan-2-yl]oxy}imidazo[1,2-b]pyridazin-3-yl)-N-ethyl-N-(2-methoxyethyl)furo[3,2-c]pyridin-4-amine;
2-(6-{[(2R)-1-Aminopropan-2-yl]oxy}imidazo[1,2-b]pyridazin-3-yl)-N-(2-methoxyethyl)-N-propylfuro[3,2-c]pyridin-4-amine;
2-(6-{[(2R)-1-Aminopropan-2-yl]oxy}imidazo[1,2-b]pyridazin-3-yl)-N-(2-methoxyethyl)-N-methylfuro[3,2-c]pyridin-4-amine;
N-Ethyl-N-(2-methoxyethyl)-2-{6-[(3S)-morpholin-3-ylmethoxy]imidazo[1,2-b]pyridazin-3-yl}furo[3,2-c]pyridin-4-amine;
3-[4-(Morpholin-4-yl)furo[3,2-c]pyridin-2-yl]-6-[(3S)-morpholin-3-ylmethoxy]imidazo[1,2-b]pyridazine;
3-{4-[(2R)-2-(Methoxymethyl)pyrrolidin-1-yl]furo[3,2-c]pyridin-2-yl}-6-[(3S)-morpholin-3-ylmethoxy]imidazo[1,2-b]pyridazine;
N-Ethyl-N-(2-methoxyethyl)-2-{6-[(3R)-morpholin-3-ylmethoxy]imidazo[1,2-b]pyridazin-3-yl}furo[3,2-c]pyridin-4-amine;
N-(2-tert-Butoxyethyl)-N-ethyl-2-{6-[(3S)-morpholin-3-ylmethoxy]imidazo[1,2-b]pyridazin-3-yl}furo[3,2-c]pyridin-4-amine;
2-[Ethyl(2-{6-[(3S)-morpholin-3-ylmethoxy]imidazo[1,2-b]pyridazin-3-yl}furo[3,2-c]pyridin-4-yl)amino]ethanol;
N-Ethyl-N-(2-methoxyethyl)-2-[6-(piperidin-2-ylmethoxy)imidazo[1,2-b]pyridazin-3-yl]furo[3,2-c]pyridin-4-amine;
6-[3-(Methylsulfonyl)propoxy]-3-[4-(morpholin-4-yl)furo[3,2-c]pyridin-2-yl]imidazo[1,2-b]pyridazine;
3-[4-(4-Methylpiperazin-1-yl)furo[3,2-c]pyridin-2-yl]-6-[3-(methylsulfonyl)propoxy]-imidazo[1,2-b]pyridazine;
6-[3-(Methylsulfonyl)propoxy]-3-[4-(pyrrolidin-1-yl)furo[3,2-c]pyridin-2-yl]imidazo[1,2-b]pyridazine;
6-[3-(Methylsulfonyl)propoxy]-3-[4-(piperidin-1-yl)furo[3,2-c]pyridin-2-yl]imidazo[1,2-b]pyridazine;
(3R)—N,N-Dimethyl-1-(2-{6-[3-(methylsulfonyl)propoxy]imidazo[1,2-b]pyridazin-3-yl}furo[3,2-c]pyridin-4-yl)pyrrolidin-3-amine;
N-Methyl-2-{6-[3-(methylsulfonyl)propoxy]imidazo[1,2-b]pyridazin-3-yl}-N-[3-(pyrrolidin-1-yl)propyl]furo[3,2-c]pyridin-4-amine;
3-[4-(4-tert-Butylpiperazin-1-yl)furo[3,2-c]pyridin-2-yl]-6-[3-(methylsulfonyl)-propoxy]imidazo[1,2-b]pyridazine;
tert-Butyl 4-(2-{6-[3-(methylsulfonyl)propoxy]imidazo[1,2-b]pyridazin-3-yl}furo-[3,2-c]pyridin-4-yl)piperazine-1-carboxylate;
N-Ethyl-N-(2-methoxyethyl)-2-{6-[3-(methylsulfonyl)propoxy]imidazo[1,2-b]pyridazin-3-yl}furo[3,2-c]pyridin-4-amine;
1-[4-(2-{6-[3-(Methylsulfonyl)propoxy]imidazo[1,2-b]pyridazin-3-yl}furo[3,2-c]pyridin-4-yl)piperazin-1-yl]ethanone;
N-(2-Methoxyethyl)-N-methyl-2-{6-[3-(methylsulfonyl)propoxy]imidazo[1,2-b]pyridazin-3-yl}furo[3,2-c]pyridin-4-amine;
3-{4-[(2R,6S)-2,6-Dimethylmorpholin-4-yl]furo[3,2-c]pyridin-2-yl}-6-[3-(methyl-sulfonyl)propoxy]imidazo[1,2-b]pyridazine;
3-[Methyl(2-{6-[3-(methylsulfonyl)propoxy]imidazo[1,2-b]pyridazin-3-yl}furo[3,2-c]pyridin-4-yl)amino]propan-1-ol;
N,N-Dimethyl-1-(2-{6-[3-(methylsulfonyl)propoxy]imidazo[1,2-b]pyridazin-3-yl}furo[3,2-c]pyridin-4-yl)piperidin-4-amine;
6-[3-(Methylsulfonyl)propoxy]-3-[4-(4-phenylpiperazin-1-yl)furo[3,2-c]pyridin-2-yl]-imidazo[1,2-b]pyridazine;
[1-(2-{6-[3-(Methylsulfonyl)propoxy]imidazo[1,2-b]pyridazin-3-yl}furo[3,2-c]pyridin-4-yl)piperidin-4-yl]methanol;
3-{4-[(3R)-3-Methylmorpholin-4-yl]furo[3,2-c]pyridin-2-yl}-6-[3-(methylsulfonyl)-propoxy]imidazo[1,2-b]pyridazine;
3-{4-[(3S)-3-Methylmorpholin-4-yl]furo[3,2-c]pyridin-2-yl}-6-[3-(methylsulfonyl)-propoxy]imidazo[1,2-b]pyridazine;
3-{4-[(2S)-2-Methylmorpholin-4-yl]furo[3,2-c]pyridin-2-yl}-6-[3-(methylsulfonyl)-propoxy]imidazo[1,2-b]pyridazine;
(5R)-5-[({3-[4-(4-Methylpiperazin-1-yl)furo[3,2-c]pyridin-2-yl]imidazo[1,2-b]pyridazin-6-yl}oxy)methyl]pyrrolidin-2-one;
(5R)-5-[({3-[4-(Piperidin-1-yl)furo[3,2-c]pyridin-2-yl]imidazo[1,2-b]pyridazin-6-yl}-oxy)methyl]pyrrolidin-2-one;
(5R)-5-[({3-[4-(4-tert-Butylpiperazin-1-yl)furo[3,2-c]pyridin-2-yl]imidazo[1,2-b]pyridazin-6-yl}oxy)methyl]pyrrolidin-2-one;
(5R)-5-{[(3-{4-[(3R)-3-(Dimethylamino)pyrrolidin-1-yl]furo[3,2-c]pyridin-2-yl}imidazo[1,2-b]pyridazin-6-yl)oxy]methyl}pyrrolidin-2-one;
(5R)-5-{[(3-{4-[Methyl(1-methylpiperidin-4-yl)amino]furo[3,2-c]pyridin-2-yl}imidazo[1,2-b]pyridazin-6-yl)oxy]methyl}pyrrolidin-2-one;
(5R)-5-{[(3-{4-[(2R)-2-({[tert-Butyl(dimethyl)silyl]oxy}methyl)pyrrolidin-1-yl]furo[3,2-c]pyridin-2-yl}imidazo[1,2-b]pyridazin-6-yl)oxy]methyl}pyrrolidin-2-one;
(5R)-5-{[(3-{4-[(2R)-2-(Hydroxymethyl)pyrrolidin-1-yl]furo[3,2-c]pyridin-2-yl}imidazo[1,2-b]pyridazin-6-yl)oxy]methyl}pyrrolidin-2-one;
(5R)-5-[({3-[4-(Morpholin-4-yl)furo[3,2-c]pyridin-2-yl]imidazo[1,2-b]pyridazin-6-yl}-oxy)methyl]pyrrolidin-2-one;
(5R)-5-{[(3-{4-[Ethyl(2-methoxyethyl)amino]furo[3,2-c]pyridin-2-yl}imidazo[1,2-b]pyridazin-6-yl)oxy]methyl}pyrrolidin-2-one;
tert-Butyl 4-[2-(6-{[(2R)-5-oxopyrrolidin-2-yl]methoxy}imidazo[1,2-b]pyridazin-3-yl)furo[3,2-c]pyridin-4-yl]piperazine-1-carboxylate;
(5R)-5-[({3-[4-(Piperazin-1-yl)furo[3,2-c]pyridin-2-yl]imidazo[1,2-b]pyridazin-6-yl}-oxy)methyl]pyrrolidin-2-one;
(5R)-5-{[(3-{4-[(2R,6S)-2,6-Dimethylmorpholin-4-yl]furo[3,2-c]pyridin-2-yl}imidazo[1,2-b]pyridazin-6-yl)oxy]methyl}pyrrolidin-2-one;
(5R)-5-{[(3-{4-[(3-Hydroxypropyl)(methyl)amino]furo[3,2-c]pyridin-2-yl}imidazo-[1,2-b]pyridazin-6-yl)oxy]methyl}pyrrolidin-2-one;
(5R)-5-{[(3-{4-[4-(Dimethylamino)piperidin-1-yl]furo[3,2-c]pyridin-2-yl}imidazo-[1,2-b]pyridazin-6-yl)oxy]methyl}pyrrolidin-2-one;
(5R)-5-{[(3-{4-[(3S)-3-(Dimethylamino)pyrrolidin-1-yl]furo[3,2-c]pyridin-2-yl}imidazo[1,2-b]pyridazin-6-yl)oxy]methyl}pyrrolidin-2-one;
(5R)-5-[({3-[4-(4-Phenylpiperazin-1-yl)furo[3,2-c]pyridin-2-yl]imidazo[1,2-b]pyridazin-6-yl}oxy)methyl]pyrrolidin-2-one;
(5R)-5-{[(3-{4-[4-(Hydroxymethyl)piperidin-1-yl]furo[3,2-c]pyridin-2-yl}imidazo-[1,2-b]pyridazin-6-yl)oxy]methyl}pyrrolidin-2-one;
(5R)-5-{[(3-{4-[(2R)-2-(Methoxymethyl)pyrrolidin-1-yl]furo[3,2-c]pyridin-2-yl}imidazo[1,2-b]pyridazin-6-yl)oxy]methyl}pyrrolidin-2-one;
(5R)-5-{[(3-{4-[(3R)-3-Methylmorpholin-4-yl]furo[3,2-c]pyridin-2-yl}imidazo[1,2-b]pyridazin-6-yl)oxy]methyl}pyrrolidin-2-one;
(5R)-5-{[(3-{4-[(2-tert-Butoxyethyl)(ethyl)amino]furo[3,2-c]pyridin-2-yl}imidazo[1,2-b]pyridazin-6-yl)oxy]methyl}pyrrolidin-2-one;
(5R)-5-{[(3-{4-[(2-Methoxyethyl)(methyl)amino]furo[3,2-c]pyridin-2-yl}imidazo [1,2-b]pyridazin-6-yl)oxy]methyl}pyrrolidin-2-one;
(5R)-5-{[(3-{4-[(2-Methoxyethyl)(propyl)amino]furo[3,2-c]pyridin-2-yl}imidazo-[1,2-b]pyridazin-6-yl)oxy]methyl}pyrrolidin-2-one;
(5R)-5-{[(3-{4-[(2S)-2-(Methoxymethyl)pyrrolidin-1-yl]furo[3,2-c]pyridin-2-yl}imidazo[1,2-b]pyridazin-6-yl)oxy]methyl}pyrrolidin-2-one;
(5R)-5-{[(3-{4-[(3S)-3-Methylmorpholin-4-yl]furo[3,2-c]pyridin-2-yl}imidazo[1,2-b]pyridazin-6-yl)oxy]methyl}pyrrolidin-2-one;
(5S)-5-{[(3-{4-[(3R)-3-Methylmorpholin-4-yl]furo[3,2-c]pyridin-2-yl}imidazo[1,2-b]pyridazin-6-yl)oxy]methyl}pyrrolidin-2-one;
(5S)-5-{[(3-{4-[Ethyl(2-methoxyethyl)amino]furo[3,2-c]pyridin-2-yl}imidazo[1,2-b]pyridazin-6-yl)oxy]methyl}pyrrolidin-2-one;
(5S)-5-{[(3-{4-[(3S)-3-Methylmorpholin-4-yl]furo[3,2-c]pyridin-2-yl}imidazo[1,2-b]pyridazin-6-yl)oxy]methyl}pyrrolidin-2-one;
(5S)-5-{[(3-{4-[(2S)-2-Methylmorpholin-4-yl]furo[3,2-c]pyridin-2-yl}imidazo[1,2-b]pyridazin-6-yl)oxy]methyl}pyrrolidin-2-one;
(6R)-6-[({3-[4-(Morpholin-4-yl)furo[3,2-c]pyridin-2-yl]imidazo[1,2-b]pyridazin-6-yl}-oxy)methyl]piperazin-2-one;
(6R)-6-{[(3-{4-[Ethyl(2-methoxyethyl)amino]furo[3,2-c]pyridin-2-yl}imidazo[1,2-b]pyridazin-6-yl)oxy]methyl}piperazin-2-one;
6-Methoxy-3-[4-(4-phenylpiperazin-1-yl)furo[3,2-c]pyridin-2-yl]imidazo[1,2-b]pyridazine;
6-Methoxy-3-{4-[(3R)-3-methylmorpholin-4-yl]furo[3,2-c]pyridin-2-yl}imidazo-[1,2-b]pyridazine;
6-Methoxy-3-{4-[(3S)-3-methylmorpholin-4-yl]furo[3,2-c]pyridin-2-yl}imidazo-[1,2-b]pyridazine;
6-Methoxy-3-{4-[(2S)-2-methylmorpholin-4-yl]furo[3,2-c]pyridin-2-yl}imidazo-[1,2-b]pyridazine;
trans-3-({3-[4-(Morpholin-4-yl)furo[3,2-c]pyridin-2-yl]imidazo[1,2-b]pyridazin-6-yl}-oxy)cyclobutanamine;
cis-3-({3-[4-(4-Methylpiperazin-1-yl)furo[3,2-c]pyridin-2-yl]imidazo[1,2-b]pyridazin-6-yl}oxy)cyclobutanamine;
2-{6-[(trans-3-Aminocyclobutyl)oxy]imidazo[1,2-b]pyridazin-3-yl}-N-methyl-N43-(pyrrolidin-1-yl)propyl]furo[3,2-c]pyridin-4-amine;
(3R)-1-(2-{6-[(trans-3-Aminocyclobutyl)oxy]imidazo[1,2-b]pyridazin-3-yl}furo-[3,2-c]pyridin-4-yl)-N,N-dimethylpyrrolidin-3-amine;
trans-3-({3-[4-(4-tert-Butylpiperazin-1-yl)furo[3,2-c]pyridin-2-yl]imidazo[1,2-b]pyridazin-6-yl}oxy)cyclobutanamine;
2-{6-[(trans-3-Aminocyclobutyl)oxy]imidazo[1,2-b]pyridazin-3-yl}-N-methyl-N-(1-methylpiperidin-4-yl)furo[3,2-c]pyridin-4-amine;
2-{6-[(trans-3-Aminocyclobutyl)oxy]imidazo[1,2-b]pyridazin-3-yl}-N-ethyl-N-(2-methoxyethyl)furo[3,2-c]pyridin-4-amine;
[(2R)-1-(2-{6-[(trans-3-Aminocyclobutyl)oxy]imidazo[1,2-b]pyridazin-3-yl}furo-[3,2-c]pyridin-4-yl)pyrrolidin-2-yl]methanol;
trans-3-({3-[4-(Piperazin-1-yl)furo[3,2-c]pyridin-2-yl]imidazo[1,2-b]pyridazin-6-yl}-oxy)cyclobutanamine;
trans-3-[(3-{4-[(2R,6S)-2,6-Dimethylmorpholin-4-yl]furo[3,2-c]pyridin-2-yl}imidazo[1,2-b]pyridazin-6-yl)oxy]cyclobutanamine;
3-[(2-{6-[(trans-3-Aminocyclobutyl)oxy]imidazo[1,2-b]pyridazin-3-yl}furo[3,2-c]pyridin-4-yl)(methyl)amino]propan-1-ol;
trans-3-({3-[4-(4-Phenylpiperazin-1-yl)furo[3,2-c]pyridin-2-yl]imidazo[1,2-b]pyridazin-6-yl}oxy)cyclobutanamine;
trans-3-[(3-{4-[(3R)-3-Methylmorpholin-4-yl]furo[3,2-c]pyridin-2-yl}imidazo[1,2-b]pyridazin-6-yl)oxy]cyclobutanamine;
2-{6-[(trans-3-Aminocyclobutyl)oxy]imidazo[1,2-b]pyridazin-3-yl}-N-(2-tert-butoxyethyl)-N-ethylfuro[3,2-c]pyridin-4-amine;
trans-3-[(3-{4-[(3S)-3-Methylmorpholin-4-yl]furo[3,2-c]pyridin-2-yl}imidazo[1,2-b]pyridazin-6-yl)oxy]cyclobutanamine;
trans-3-[(3-{4-[(2S)-2-Methylmorpholin-4-yl]furo[3,2-c]pyridin-2-yl}imidazo[1,2-b]pyridazin-6-yl)oxy]cyclobutanamine;
2-(6-{[(1S,2S)-1-Amino-2,3-dihydro-1H-inden-2-yl]oxy}imidazo[1,2-b]pyridazin-3-yl)-N-ethyl-N-(2-methoxyethyl)furo[3,2-c]pyridin-4-amine;
2-(6-{[(1R,2S)-1-Amino-2,3-dihydro-1H-inden-2-yl]oxy}imidazo[1,2-b]pyridazin-3-yl)-N-ethyl-N-(2-methoxyethyl)furo[3,2-c]pyridin-4-amine;
(6S)-6-{[(3-{4-[Ethyl(2-methoxyethyl)amino]furo[3,2-c]pyridin-2-yl}imidazo[1,2-b]pyridazin-6-yl)oxy]methyl}piperazin-2-one.
(3S)—N,N-Dimethyl-1-(2-{6-[3-(methylsulfonyl)propoxy]imidazo[1,2-b]pyridazin-3-yl}furo[3,2-c]pyridin-4-yl)pyrrolidin-3-amine;
(3S)-1-[2-(6-{[(2S)-2-Aminopropyl]oxy}imidazo[1,2-b]pyridazin-3-yl)furo[3,2-c]pyridin-4-yl]-N,N-dimethylpyrrolidin-3-amine;
(2S)-1-({3-[4-(4-tert-Butylpiperazin-1-yl)furo[3,2-c]pyridin-2-yl]imidazo[1,2-b]pyridazin-6-yl}oxy)propan-2-amine;
(2S)-1-[(3-{4-[(2R)-2-Methylmorpholin-4-yl]furo[3,2-c]pyridin-2-yl}imidazo[1,2-b]pyridazin-6-yl)oxy]propan-2-amine;
(5R)-5-{[(3-{4-[(2R)-2-Methylmorpholin-4-yl]furo[3,2-c]pyridin-2-yl}imidazo[1,2-b]pyridazin-6-yl)oxy]methyl}pyrrolidin-2-one;
3-{4-[(2R)-2-Methylmorpholin-4-yl]furo[3,2-c]pyridin-2-yl}-6-[3-(methylsulfonyl)-propoxy]imidazo[1,2-b]pyridazine;
6-Methoxy-3-{4-[(2R)-2-methylmorpholin-4-yl]furo[3,2-c]pyridin-2-yl}imidazo[1,2-b]pyridazine;
(2R)-1-[(3-{4-[(2R)-2-Methylmorpholin-4-yl]furo[3,2-c]pyridin-2-yl}imidazo[1,2-b]pyridazin-6-yl)oxy]propan-2-amine;
trans-3-[(3-{4-[(2R)-2-Methylmorpholin-4-yl]furo[3,2-c]pyridin-2-yl}imidazo[1,2-b]pyridazin-6-yl)oxy]cyclobutanamine;
(5R)-5-{[(3-{4-[(2S)-2-Methylmorpholin-4-yl]furo[3,2-c]pyridin-2-yl}imidazo[1,2-b]pyridazin-6-yl)oxy]methyl}pyrrolidin-2-one;
(2R)-1-({3-[4-(2,2-Dimethylmorpholin-4-yl)furo[3,2-c]pyridin-2-yl]imidazo[1,2-b]pyridazin-6-yl}oxy)propan-2-amine;
(5S)-5-{[(3-{4-[(2R)-2-Methylmorpholin-4-yl]furo[3,2-c]pyridin-2-yl}imidazo[1,2-b]pyridazin-6-yl)oxy]methyl}pyrrolidin-2-one;
3-[4-(3,3-Dimethylpiperazin-1-yl)furo[3,2-c]pyridin-2-yl]-6-(2,2,2-trifluoroethoxy)-imidazo[1,2-b]pyridazine;
3-{4-[(3R)-3-Methylpiperazin-1-yl]furo[3,2-c]pyridin-2-yl}-6-(2,2,2-trifluoroethoxy)-imidazo[1,2-b]pyridazine;
(2S)-1-({3-[4-(2,2-Dimethylmorpholin-4-yl)furo[3,2-c]pyridin-2-yl]imidazo[1,2-b]pyridazin-6-yl}oxy)propan-2-amine;
trans-3-({3-[4-(2,2-Dimethylmorpholin-4-yl)furo[3,2-c]pyridin-2-yl]imidazo[1,2-b]pyridazin-6-yl}oxy)cyclobutanamine;
3-[4-(3,3-Dimethylpiperazin-1-yl)furo[3,2-c]pyridin-2-yl]-6-methoxyimidazo[1,2-b]pyridazine
3-[4-(3,3-Dimethylpiperazin-1-yl)furo[3,2-c]pyridin-2-yl]-6-[3-(methylsulfonyl)-propoxy]imidazo[1,2-b]pyridazine;
(5S)-5-[({3-[4-(2,2-Dimethylmorpholin-4-yl)furo[3,2-c]pyridin-2-yl]imidazo[1,2-b]pyridazin-6-yl}oxy)methyl]pyrrolidin-2-one;
6-Methoxy-3-{4-[(3S)-3-methylpiperazin-1-yl]furo[3,2-c]pyridin-2-yl}imidazo[1,2-b]pyridazine
(5S)-5-[({3-[4-(3,3-Dimethylpiperazin-1-yl)furo[3,2-c]pyridin-2-yl]imidazo[1,2-b]pyridazin-6-yl}oxy)methyl]pyrrolidin-2-one;
3-[4-(2,2-Dimethylmorpholin-4-yl)furo[3,2-c]pyridin-2-yl]-6-[3-(methylsulfonyl)-propoxy]imidazo[1,2-b]pyridazine;
(2R)-2-[(3-{4-[(2S)-2-(Methoxymethyl)pyrrolidin-1-yl]furo[3,2-c]pyridin-2-yl}imidazo[1,2-b]pyridazin-6-yl)oxy]propan-1-amine;
[{3-[(3-{4-[Ethyl(2-methoxyethyl)amino]furo[3,2-c]pyridin-2-yl}imidazo[1,2-b]pyridazin-6-yl)oxy]propyl}(methyl)oxido-λ⁶-sulfanylidene]cyanamide;
3-[4-(Morpholin-4-yl)furo[3,2-c]pyridin-2-yl]-6-[(2S)-pyrrolidin-2-ylmethoxy]imidazo[1,2-b]pyridazine;
N-Ethyl-N-(2-methoxyethyl)-2-{6-[(2S)-pyrrolidin-2-ylmethoxy]imidazo[1,2-b]pyridazin-3-yl}furo[3,2-c]pyridin-4-amine;
3-[4-(Morpholin-4-yl)furo[3,2-c]pyridin-2-yl]-6-(piperidin-2-ylmethoxy)imidazo[1,2-b]pyridazine;
(5R)-5-[({3-[4-(3,3-Dimethylpiperazin-1-yl)furo[3,2-c]pyridin-2-yl]imidazo[1,2-b]pyridazin-6-yl}oxy)methyl]pyrrolidin-2-one;
(5R)-5-[({3-[4-(2,2-Dimethylmorpholin-4-yl)furo[3,2-c]pyridin-2-yl]imidazo[1,2-b]pyridazin-6-yl}oxy)methyl]pyrrolidin-2-one;
3-{4-[(3S)-3-Methylpiperazin-1-yl]furo[3,2-c]pyridin-2-yl}-6-[3-(methylsulfonyl)-propoxy]imidazo[1,2-b]pyridazine;
6-Methoxy-3-{4-[(3R)-3-methylpiperazin-1-yl]furo[3,2-c]pyridin-2-yl}imidazo[1,2-b]-pyridazine;
3-{4-[(3S)-3-Methylpiperazin-1-yl]furo[3,2-c]pyridin-2-yl}-6-(2,2,2-trifluoroethoxy)-imidazo[1,2-b]pyridazine;
3-[4-(2,2-Dimethylmorpholin-4-yl)furo[3,2-c]pyridin-2-yl]-6-methoxyimidazo[1,2-b]-pyridazine;
1-[(2S)-2-(2-{[3-(Furo[3,2-c]pyridin-2-yl)imidazo[1,2-b]pyridazin-6-yl]oxy}ethyl)pyrrolidin-1-yl]ethanone;
5-(2-{[3-(4-Methoxyfuro[3,2-c]pyridin-2-yl)imidazo[1,2-b]pyridazin-6-yl]oxy}ethyl)pyrrolidin-2-one;
(5S)-5-({[3-(4-Methoxyfuro[3,2-c]pyridin-2-yl)imidazo[1,2-b]pyridazin-6-yl]oxy}-methyl)pyrrolidin-2-one;
(5R)-5-[({3-[4-(Propan-2-yloxy)furo[3,2-c]pyridin-2-yl]imidazo[1,2-b]pyridazin-6-yl}-oxy)methyl]pyrrolidin-2-one;
(5R)-5-[({3-[4-(2,2-Dimethylpropoxy)furo[3,2-c]pyridin-2-yl]imidazo[1,2-b]-pyridazin-6-yl}oxy)methyl]pyrrolidin-2-one;
(5R)-5-({[3-(4-Methoxyfuro[3,2-c]pyridin-2-yl)imidazo[1,2-b]pyridazin-6-yl]-oxy}-methyl)pyrrolidin-2-one;
1-[(2S)-2-(2-{[3-(Furo[3,2-c]pyridin-2-yl)imidazo[1,2-b]pyridazin-6-yl]-oxy}ethyl)pyrrolidin-1-yl]-2,2-dimethylpropan-1-one;
Cyclopropyl[(2R)-2-({[3-(4-methoxyfuro[3,2-c]pyridin-2-yl)imidazo[1,2-b]-pyridazin-6-yl]oxy}methyl)morpholin-4-yl]methanone;
6-({[3-(4-Methoxyfuro[3,2-c]pyridin-2-yl)imidazo[1,2-b]pyridazin-6-yl]oxy}methyl)piperidin-2-one;
2,2,2-Trifluoro-1-[(2R)-2-({[3-(4-methoxyfuro[3,2-c]pyridin-2-yl)imidazo-[1,2-b]-pyridazin-6-yl]oxy}methyl)morpholin-4-yl]ethanone;
1-[(2R)-2-({[3-(4-Methoxyfuro[3,2-c]pyridin-2-yl)imidazo[1,2-b]pyridazin-6-yl]oxy}-methyl)morpholin-4-yl]-2,2-dimethylpropan-1-one;
(5R)-5-[({3-[4-(Cyclopropylmethoxy)furo[3,2-c]pyridin-2-yl]imidazo[1,2-b]-pyridazin-6-yl}oxy)methyl]pyrrolidin-2-one;
(6R)-6-[({3-[4-(Propan-2-yloxy)furo[3,2-c]pyridin-2-yl]imidazo[1,2-b]pyridazin-6-yl}-oxy)methyl]piperazin-2-one; and
(5R)-5-[({3-[4-(Ethylamino)furo[3,2-c]pyridin-2-yl]imidazo[1,2-b]pyridazin-6-yl}oxy)-methyl]pyrrolidin-2-one.

10. A method of preparing a compound of general formula (Ia) according to any one of claims 1 to 6, said method comprising the step of allowing an intermediate compound of general formula (Ea): 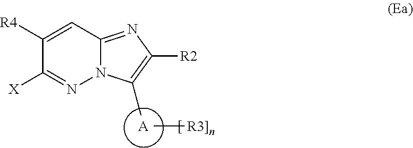
in which A, R2, R3, R4 and n are as defined for the compound of general formula (Ia) according to any one of claims 1 to 6, and X represents a leaving group, such as a halogen atom, for example a chlorine, bromine or iodine atom, or a perfluoroalkylsulfonate group for example, such as a trifluoromethylsulfonate group or a nonafluorobutylsulfonate group, for example,
to react with a compound of general formula (II): 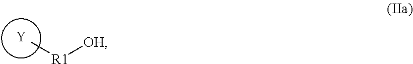
in which R1 and Y are as defined for the compound of general formula (Ia) according to any one of claims 1 to 6,
thereby giving a compound of general formula (Ia): 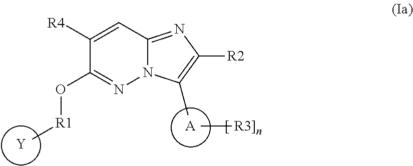
in which A, Y, R1, R2, R3, R4 and n are as defined for the compound of general formula (Ia) according to any one of claims 1 to 6.

11. A compound of general formula (I), or a stereoisomer, a tautomer, an N-oxide, a hydrate, a solvate, or a salt thereof, particularly a pharmaceutically acceptable salt thereof, or a mixture of same, according to any one of claims 1 to 9, for use in the treatment or prophylaxis of a disease.

12. A pharmaceutical composition comprising a compound of general formula (I), or a stereoisomer, a tautomer, an N-oxide, a hydrate, a solvate, or a salt thereof, particularly a pharmaceutically acceptable salt thereof, or a mixture of same, according to any one of claims 1 to 9, and a pharmaceutically acceptable diluent or carrier.

13. A pharmaceutical combination comprising:
one or more first active ingredients selected from a compound of general formula (I) according to any of claims 1 to 9, and
one or more second active ingredients selected from chemotherapeutic anti-cancer agents and target-specific anti-cancer agents.

14. Use of a compound of general formula (I), or a stereoisomer, a tautomer, an N-oxide, a hydrate, a solvate, or a salt thereof, particularly a pharmaceutically acceptable salt thereof, or a mixture of same, according to any one of claims 1 to 9, for the prophylaxis or treatment of a disease.

15. Use of a compound of general formula (I), or a stereoisomer, a tautomer, an N-oxide, a hydrate, a solvate, or a salt thereof, particularly a pharmaceutically acceptable salt thereof, or a mixture of same, according to any one of claims 1 to 9, for the preparation of a medicament for the prophylaxis or treatment of a disease.

16. Use according to claim 11, 14 or 15, wherein said disease is a disease of uncontrolled cell growth, proliferation and/or survival, an inappropriate cellular immune response, or an inappropriate cellular inflammatory response, particularly in which the uncontrolled cell growth, proliferation and/or survival, inappropriate cellular immune response, or inappropriate cellular inflammatory response is mediated by the MKNK-1 pathway, more particularly in which the disease of uncontrolled cell growth, proliferation and/or survival, inappropriate cellular immune response, or inappropriate cellular inflammatory response is a haematological tumour, a solid tumour and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

17. Use of a compound of general formula (V) for the preparation of a compound of general formula (I) according to any one of claims 1 to 9. 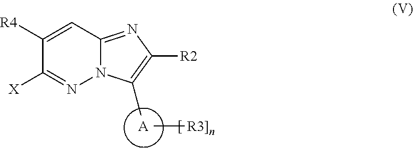
in which A, R2, R3, R4 and n are as defined for the compound of general formula (I) according to any one of claims 1 to 9, and X represents a leaving group, such as a halogen atom, for example a chlorine, bromine or iodine atom, or a perfluoroalkylsulfonate group for example, such as a trifluoromethylsulfonate group or a nonafluorobutylsulfonate group, for example,
for the preparation of a compound of general formula (I) according to any one of claims 1 to 9.
